(19) United States
(12) Patent Application Publication
(10) Pub. No.: US 2005/0181375 A1
(43) Pub. Date: Aug. 18, 2005

(54) Novel methods of diagnosis of metastatic cancer, compositions and methods of screening for modulators of metastatic cancer

(76) Inventors: Natasha Aziz, Palo Alto, CA (US); Albert Zlotnik, San Diego, CA (US)

Correspondence Address: HOWREY SIMON ARNOLD & WHITE, LLP;C/O M.P. DROSOS, DIRECTOR OF IP ADMINISTRATION, 2941 FAIRVIEW PK, BOX 7, FALLS CHURCH, VA, 22042, US

(21) Appl. No.: 10/756,149

(22) Filed: Jan. 12, 2004

Related U.S. Application Data
(60) Provisional application No. 60/439,058, filed on Jan. 10, 2003.

(51) Int. Cl. C12Q 1/68; G01N 33/574; C07K 16/40; C07H 21/04; C12N 9/00

(52) U.S. Cl. 435/6; 435/7.23; 536/23.2; 435/183; 530/388.8

(57) ABSTRACT

Described herein are methods and compositions that can be used for diagnosis and treatment of metastatic cancer. Also described herein are methods that can be used to identify modulators of metastatic cancer.

### RELATED APPLICATIONS

This application claims priority from U.S. Provisional Application No. 60/439,058 filed Jan. 10, 2003, which is hereby incorporated by reference in its entirety.

### SEQUENCE LISTING SUBMITTED ON CD

This application includes a sequence listing on a compact disc submitted with this application. The compact disc includes a 20.724 megabyte ASCII formatted file, created Jan. 9, 2004, entitled, “0188SEQL.txt”. This file lists 5818 sequences. In accordance with 37 C.F.R. § 1.52(e)(5), the sequence listing on the compact disc is hereby incorporated by reference.

### FIELD OF THE INVENTION

The invention relates to the identification of nucleic acids and proteins identified by expression profiles, and nucleic acids, products, and antibodies thereto that are involved in metastatic cancer; and to the use of such expression profiles and compositions in diagnosis and therapy of metastatic cancer. The invention further relates to methods for identifying and using agents and/or targets that inhibit metastatic cancer.

### BACKGROUND OF THE INVENTION

Metastatic disease can be viewed as two simultaneously occurring diseases; a disease at a primary site, and a related disease at a secondary location distant from the primary site. Each disease may have different mortality rates, for example in cases of metastatic breast or lung cancer to the brain. In such cases untreated brain metastases are rapidly fatal, while primary breast or lung cancer may actually be cureable.

Unfortunately, many cancers metastasize. While there are many variables that determine where metastatic tumors grow, often, the metastatic location is the nearest cluster of small blood vessels found by the circulating cancer cells. Thus, lung cancer commonly metastasizes to the brain; colon cancer commonly metastasizes to the liver. Alternatively, the cancer may have a preferred site of metastasis. For example, the brain is a preferred site for melanoma and small cell lung cancer. A metastasis of a metastasis may develop as well. For example, a colon cancer may metastasize to the liver, which in turn may metastasize to the lung, which may in turn metastasize to the brain.

Without wishing to be bound by theory, it is believed that metastasis occurs when cancer cells from the primary site break away and enter the body's circulatory system through the blood stream, lymph system, or spinal fluid and travel to distant locations. Although cancer metastasis may occur in nearly any organ, brain metastases are one of the most common sites of systemic spread from solid tumors, with an annual incidence of over 100,000 cases. Indeed, metastatic brain tumors occur in about one-fourth of all cancers that metastasize, and primary and metastatic brain tumors kill 15,000 people each year. The most common primary tumors that metastasize to the brain are lung, breast, melanoma, and colon, however almost any cancer has this potential.

Although almost 1 in 4 patients with cancer will develop tumors that spread to the central nervous system (CNS) cancer can metastasize to almost any organ.

Classification of metastatic tumors depends on the tissue type from which they are derived. Unfortunately, it is often difficult or impossible to determine the location of the primary cancer and this can unnecessarily complicate diagnosis and treatment of the metastatic cancer. In addition early diagnosis of metastatic cancer can greatly improve the prognostic outlook for a cancer patient. Often metastatic burden, rather than the primary cancer, is what ultimately kills a patient.

Thus need exists for an efficient and effective method for the identification of metastatic tumor origins, as well as methods for diagnosis, prognosis and treatment of metastatic cancer. The development of successful therapeutic modalities is however, unlikely to follow the conventional approaches of surgery, radiation and cytotoxic chemotherapy. Rather, the best hope lies in the rapidly expanding field of molecular medicine.

Accordingly, provided herein are molecular targets for therapeutic intervention in metastatic breast and lung cancer. Additionally, provided herein are methods that can be used in diagnosis and prognosis of metastatic breast and lung cancer. Further provided are methods that can be used to screen candidate bioactive agents for the ability to modulate metastatic cancer including metastatic brain tumors.

### SUMMARY OF THE INVENTION

The present invention therefore provides nucleotide sequences of genes that are up- and down-regulated in metastatic breast or metastatic lung cancer cells. Such genes and the proteins they encode are useful for diagnostic and prognostic purposes, and also as targets for screening for therapeutic compounds that modulate metastatic breast or lung cancer, such as antibodies. The methods of detecting nucleic acids of the invention or their encoded proteins can be used for a number of purposes. Examples include, early detection of breast or lung cancers, monitoring and early detection of relapse following treatment of breast or lung cancers including early detection of metastatic cancer, monitoring response to therapy of breast or lung cancers, determining prognosis of breast or lung cancers, directing therapy of breast or lung cancers, selecting patients for postoperative chemotherapy or radiation therapy, selecting therapy, determining tumor prognosis and the likelihood that a given cancer will metastasize or has metastasized, treatment, or response to treatment, early detection of precancerous conditions and early detection of metastasis. Other aspects of the invention will become apparent to the skilled artisan by the following description of the invention.

In one aspect, the present invention provides a method of detecting a metastatic breast or lung cancer-associated transcript in a cell from a patient, the method comprising contacting a biological sample from the patient with a polynucleotide that selectively hybridizes to a sequence at least 80% identical to a sequence as shown in Tables 1A-12C.

In one embodiment, the polynucleotide selectively hybridizes to a sequence at least 95% identical to a sequence as shown in Tables 1A-12C. In another embodiment, the polynucleotide comprises a sequence as shown in Tables 1A-12C.

In one embodiment, the biological sample is a tissue sample. In another embodiment, the biological sample comprises isolated nucleic acids, e.g., mRNA.

In one embodiment, the polynucleotide is labeled, e.g., with a fluorescent label.

In one embodiment, the polynucleotide is immobilized on a solid surface.

In one embodiment, the patient is undergoing a therapeutic regimen to treat metastatic breast or lung cancer. In another embodiment, the metastatic breast or lung cancer has metastasized to the brain.

In one embodiment, the patient is a human.

In one embodiment, the method further comprises the step of amplifying nucleic acids before the step of contacting the biological sample with the polynucleotide.

In another aspect, the present invention provides methods of detecting polypeptide encoded by a metastatic breast or lung cancer-associated transcript in a cell from a patient, the method comprising contacting a biological sample from the patient with an antibody that specifically binds a polypeptide encoded by a sequence at least 80% identical to a sequence as shown in Tables 1A-12C.

In another aspect, the present invention provides a method of monitoring the efficacy of a therapeutic treatment of metastatic breast or lung cancer, the method comprising the steps of: (i) providing a biological sample from a patient undergoing the therapeutic treatment; and (ii) determining the level of a metastatic breast or lung cancer-associated transcript in the biological sample by contacting the biological sample with a polynucleotide that selectively hybridizes to a sequence at least 80% identical to a sequence as shown in Tables 1A-12C., thereby monitoring the efficacy of the therapy.

In one embodiment, the method further comprises the step of: (iii) comparing the level of the metastatic breast or lung cancer-associated transcript to a level of the metastatic breast or lung cancer-associated transcript in a biological sample from the patient prior to, or earlier in, the therapeutic treatment.

In another aspect, the present invention provides a method of monitoring the efficacy of a therapeutic treatment of metastatic breast or lung cancer, the method comprising the steps of: (i) providing a biological sample from a patient undergoing the therapeutic treatment; and (ii) determining the level of a metastatic breast or lung cancer-associated antibody in the biological sample by contacting the biological sample with a polypeptide encoded by a polynucleotide that selectively hybridizes to a sequence at least 80% identical to a sequence as shown in Tables 1A-12C, wherein the polypeptide specifically binds to the metastatic breast or lung cancer-associated antibody, thereby monitoring the efficacy of the therapy.

In one embodiment, the method further comprises the step of: (iii) comparing the level of the metastatic breast or lung cancer-associated antibody to a level of the metastatic breast or lung cancer-associated antibody in a biological sample from the patient prior to, or earlier in, the therapeutic treatment.

In another aspect, the present invention provides a method of monitoring the efficacy of a therapeutic treatment of metastatic breast or lung cancer, the method comprising the steps of: (i) providing a biological sample from a patient undergoing the therapeutic treatment; and (ii) determining the level of a metastatic breast or lung cancer-associated polypeptide in the biological sample by contacting the biological sample with an antibody, wherein the antibody specifically binds to a polypeptide encoded by a polynucleotide that selectively hybridizes to a sequence at least 80% identical to a sequence as shown in Tables 1A-12C, thereby monitoring the efficacy of the therapy.

In one embodiment, the method further comprises the step of: (iii) comparing the level of the metastatic breast or lung cancer-associated polypeptide to a level of the metastatic breast or lung cancer-associated polypeptide in a biological sample from the patient prior to, or earlier in, the therapeutic treatment.

In one aspect, the present invention provides an isolated nucleic acid molecule consisting of a polynucleotide sequence as shown in Tables 1A-12C.

In one embodiment, an expression vector or cell comprises the isolated nucleic acid.

In one aspect, the present invention provides an isolated polypeptide which is encoded by a nucleic acid molecule having polynucleotide sequence as shown in Tables 1A-12C.

In another aspect, the present invention provides an antibody that specifically binds to an isolated polypeptide which is encoded by a nucleic acid molecule having polynucleotide sequence as shown in Tables 1A-12C.

In one embodiment, the antibody is conjugated to an effector component, e.g., a fluorescent label, a radioisotope or a cytotoxic chemical.

In one embodiment, the antibody is an antibody fragment. In another embodiment, the antibody is humanized.

In one aspect, the present invention provides a method of detecting a metastatic breast or lung cancer cell in a biological sample from a patient, the method comprising contacting the biological sample with an antibody as described herein.

In another aspect, the present invention provides a method of detecting antibodies specific to metastatic breast or lung cancer in a patient, the method comprising contacting a biological sample from the patient with a polypeptide encoded by a nucleic acid comprises a sequence from Tables 1A-12C.

In another aspect, the present invention provides a method for identifying a compound that modulates a metastatic breast or lung cancer-associated polypeptide, the method comprising the steps of: (i) contacting the compound with a metastatic breast or lung cancer-associated polypeptide, the polypeptide encoded by a polynucleotide that selectively hybridizes to a sequence at least 80% identical to a sequence as shown in Tables 1A-12C; and (ii) determining the functional effect of the compound upon the polypeptide.

In one embodiment, the functional effect is a physical effect, an enzymatic effect, or a chemical effect.

In one embodiment, the polypeptide is expressed in a eukaryotic host cell or cell membrane. In another embodiment, the polypeptide is recombinant.

In one embodiment, the functional effect is determined by measuring ligand binding to the polypeptide.

In another aspect, the present invention provides a method of inhibiting proliferation of a metastatic breast or lung cancer-associated cell to treat breast or lung cancer in a patient, the method comprising the step of administering to the subject a therapeutically effective amount of a compound identified as described herein.

In one embodiment, the compound is an antibody.

In another aspect, the present invention provides a drug screening assay comprising the steps of: (i) administering a test compound to a mammal having metastatic breast or lung cancer or a cell isolated therefrom; (ii) comparing the level of gene expression of a polynucleotide that selectively hybridizes to a sequence at least 80% identical to a sequence as shown in Tables 1A-12C in a treated cell or mammal with the level of gene expression of the polynucleotide in a control cell or mammal, wherein a test compound that modulates the level of expression of the polynucleotide is a candidate for the treatment of metastatic breast or lung cancer.

In one embodiment, the control is a mammal with metastatic breast or lung cancer or a cell therefrom that has not been treated with the test compound. In another embodiment, the control is a normal cell or mammal.

In another aspect, the present invention provides a method for treating a mammal having metastatic breast or lung cancer comprising administering a compound identified by the assay described herein.

In another aspect, the present invention provides a pharmaceutical composition for treating a mammal having meta static breast or lung cancer, the composition comprising a compound identified by the assay described herein and a physiologically acceptable excipient.

### DETAILED DESCRIPTION OF THE INVENTION

In accordance with the objects outlined above, the present invention provides novel methods for diagnosis and treatment of metastatic breast or metastatic lung cancer. The invention is useful for the treatment of metastatic breast and metastatic lung cancer when the cancer is metastasized to the brain, as well as when the cancer is metastasized to other organs and tissues. The invention also provides methods of screening for compositions which modulate metastatic breast cancer or metastatic lung cancer.

Primary tumors are classified by the type of tissue from which they arise, metastatic tumors are classified by the tissue type from which the cancer cells are derived. Almost any cancer can metastasize. The metastases may occur to any site, however some cancers preferentially metastasize to particular organs. For example lung, breast, head & neck, cervical, and bladder tumors frequently metastasize to particular organs. Specifically, lung cancer metastatisizes to: brain, bone, liver, adrenal glands, lung, pleura, subcutaneous tissue, kidney, lymph nodes, cerebrospinal fluid, pancreas, bone marrow. Breast cancer metastatisizes to: lymph nodes, breast, abdominal viscera, lungs, bones, liver, adrenal glands, brain, meninges, pleura, cerebrospinal fluid. Head and neck cancer metastatisizes to: lung, esophagus, upper aerodigestive tract, lymph nodes, oral cavity, nasal cavity. Cervical cancer metastatisizes to: vagina, paracervical spaces, bladder, rectum, pelvic wall, lymph nodes. Bladder cancer metastatisizes to: prostate, uterus, vagina, bowel, pelvic wall, lymph nodes, and perivesical fat.

Brain metastases are a particular concern because of the deadly nature of brain tumors in general. Because the brain is generally unforgiving in its response to both the tumor and therapy, prognosis in cases metastatic brain tumors is especially poor. This is the case whether or not the primary cancer is treatable or even cured.

Whether or not cancer cells metastasize to the brain or other parts of the body depends on many factors including the type of cancer, stage of cancer, and original location of the cancer. Treatment for secondary (metastatic) tumors depends on where the cancer started and the extent of the spread as well as other factors, including the patient's age, general health, and response to previous treatment.

Knowing the origin of metastatic cancer can greatly improve the probable outcome of treatment for individuals with metastatic disease. Indeed, the earlier metastatic cancer can be detected, the better is the prognosis for the individual since it is often metastatic burden that kills a patient. Because metastatic burden increases with time, early detection is essential for successful treatment.

Thus, in accordance with the objectives of the invention, Tables 1A-12C provide UniGene cluster identification numbers for the nucleotide sequence of genes that exhibit increased or decreased expression in metastasizing breast and lung cancer samples. Tables 1A-12C also provide an exemplar accession number that provides a nucleotide sequence that is part of the UniGene cluster.

Table 1A shows about 461 genes upregulated in breast metastases to the brain relative to normal breast tissues. Table 2A shows about 445 genes upregulated in breast metastases to the brain relative to normal body tissues. Table 3A shows about 216 genes upregulated in breast metastases to the brain relative to primary breast tumors. Table 4A shows about 350 genes downregulated in breast metastases to the brain relative to primary breast tumors. Table 5A shows about 489 genes downregulated in breast metastases to the brain relative to normal breast tissue. Table 6A shows about 1251 genes upregulated in lung metastases to the brain relative to normal lung tissues. Table 7A shows about 381 genes upregulated in lung metastases to the brain relative to normal body tissues. Table 8A shows about 330 genes upregulated in lung metastases to the brain relative to primary lung tumors. Table 9A shows about 252 genes downregulated in lung metastases to the brain relative to primary lung tumors. Table 10A shows about 289 genes downregulated in lung metastases to the brain relative to normal lung tissue. Table 11A shows about 1198 genes upregulated in breast and lung metastases to the brain relative to normal body tissues. Table 12A shows about 2867 genes upregulated in breast and lung metastases to the brain relative to normal breast and lung tissues.

Although the Tables and analysis herein is derived primarily from metastases to the brain, it is expected that markers identified from these samples should also be expressed in metastasis to other organs, particularly metastasis originating from tumors in the lung, breast, head and neck, cervix, and bladder. Indeed, the tumor-specific genes expressed in lung tumors are often also expressed in head and neck, cervical, and bladder tumors Therefore, the genes identified in metastases of primary lung tumors may also be expressed in primary tumors and metastases arising from primary tumors of the nasal cavity, paranasal sinuses, nasopharynx, oral cavity, oral pharynx, lip, larynx, hypopharynx, salivary glands, paragangliomas, esophagus, cervix, vagina, vulva, or bladder. Thus, the identified metastatic markers should be useful in diagnosis, prognosis, or therapy of metastases from these cancers.

Furthermore, one of skill will recognize that although the sequences identified in Tables 1A-12C exhibited increased or decreased expression in metastasizing breast or lung cancer samples, the sequences of the invention, and their encoded proteins, can also be used to diagnose, treat or prevent cancers in patients with non-metastatic breast cancers or non-metastatic lung cancers. Alteration of gene expression for a gene in Tables 1A-12C may be more likely or less likely to indicate that the subject will progress to metastatic disease. The sequences can also be used to diagnose, treat or prevent precancerous or benign conditions.

Alteration of gene expression for a gene in Tables 1 A-12C may or may not indicate that the subject is more likely to progress to cancer or to metastatic disease. Thus, although the specification focuses primarily on metastasizing breast or lung cancer, the methods described below can also be applied to non-metastasizing breast or lung cancers and precancerous or benign conditions as well.

### Definitions

The term “metastatic breast cancer protein” or “metastatic breast cancer polynucleotide” or “metastatic breast cancer-associated transcript” refers to nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a UniGene cluster of Tables 1-5, 11, and 12; (2) bind to antibodies, e.g., polyclonal antibodies, raised against an immunogen comprising an amino acid sequence encoded by a nucleotide sequence of or associated with a UniGene cluster of Tables 1-5, 11, and 12, and conservatively modified variants thereof; (3) specifically hybridize under stringent hybridization conditions to a nucleic acid sequence, or the complement thereof of Tables 1-5, 11, and 12 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% amino sequence identity, preferably over a region of over a region of at least about 25, 50, 100, 200, 500, 1000, or more amino acid, to an amino acid sequence encoded by a nucleotide sequence of or associated with a UniGene cluster of Tables 1-5, 11, and 12. A polynucleotide or polypeptide sequence is typically from a mammal including, but not limited to, primate, e.g., human; rodent, e.g., rat, mouse, hamster; cow, pig, horse, sheep, or other mammal. A “metastatic breast cancer polypeptide” and a “metastatic breast cancer polynucleotide,” include both naturally occurring or recombinant.

The term “metastatic lung cancer protein” or “metastatic lung cancer polynucleotide” or “metastatic lung cancer-associated transcript” refers to nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a UniGene cluster of Tables 6A-12C; (2) bind to antibodies, e.g., polyclonal antibodies, raised against an immunogen comprising an amino acid sequence encoded by a nucleotide sequence of or associated with a UniGene cluster of Tables 6A-12C, and conservatively modified variants thereof; (3) specifically hybridize under stringent hybridization conditions to a nucleic acid sequence, or the complement thereof of Tables 6A-12C and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a UniGene cluster of Tables 6A-12C. A polynucleotide or polypeptide sequence is typically from a mammal including, but not limited to, primate, e.g., human; rodent, e.g., rat, mouse, hamster; cow, pig, horse, sheep, or other mammal. A “metastatic lung cancer polypeptide” and a “metastatic lung cancer polynucleotide,” include both naturally occurring or recombinant.

The term “metastasis” refers to the process by which a disease shifts from from one part of the body to another. This process may include the spreading of neoplasms from the site of a primary tumor to distant parts of the body.

The term “metastatic brain tumor” refers to a tumor of the brain and/or its associated bone, blood vessels, meninges etc that has developed as a result of the metastasis of cancer from a primary site to the brain.

The term “secondary brain tumor” refers to a metastatic brain tumor as defined above.

The term “metastatic breast cancer” refers to any cancer in any part of the body which has its origins in breast cancer tissue. Metastatic breast cancer includes, but is not limited to “metastatic brain tumors” that have their origin in a primary breast cancer, and pre- metastatic primary tumor cells in the process of developing a metastatic phenotype.

The term “metastatic lung cancer” refers to any cancer in any part of the body which has its origins in lung cancer tissue. Metastatic lung cancer includes, but is not limited to “metastatic brain tumors” that have their origin in a primary lung cancer, and pre-metastatic primary tumor cells in the process of developing a metastatic phenotype.

A “full length” metastatic breast or lung cancer protein or nucleic acid refers to a metastatic breast or lung cancer polypeptide or polynucleotide sequence, or a variant thereof, that contains all of the elements normally contained in one or more naturally occurring, wild type metastatic breast or lung cancer polynucleotide or polypeptide sequences. The “full length” may be prior to, or after, various stages of post-translation processing or splicing, including alternative splicing.

“Biological sample” as used herein is a sample of biological tissue or fluid that contains nucleic acids or polypeptides, e.g., of a metastatic breast or lung cancer protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate, e.g., chimpanzee or human; cow; dog; cat; a rodent, e.g., guinea pig, rat, mouse; rabbit; or other mammal; or a bird; reptile; fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds., Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to essentially no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1 -2 min. Protocols and guidelines for low and high stringency amplification reactions are provided, e.g., in Innis et al., PCR Protocols, A Guide to Methods and Applications (1990).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., see, e.g., Current Protocols in Molecular Biology (Ausubel et al., eds. 1995 supplement).

The phrase “functional effects” in the context of assays for testing compounds that modulate activity of a metastatic breast cancer or metastatic lung cancer protein includes the determination of a parameter that is indirectly or directly under the influence of the metastatic breast cancer or metastatic lung cancer protein or nucleic acid, e.g., an enzymatic, functional, physical, or chemical effect, such as the ability to decrease metastatic breast cancer or metastatic lung cancer. It includes ligand binding activity; cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of metastatic breast cancer or metastatic lung cancer cells. “Functional effects” include in vitro, in vivo, and ex vivo activities.

By “determining the functional effect” is meant assaying for a compound that increases or decreases a parameter that is indirectly or directly under the influence of a metastatic breast cancer or metastatic lung cancer protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the metastatic breast cancer or metastatic lung cancer protein; measuring binding activity or binding assays, e.g., binding to antibodies or other ligands, and measuring cellular proliferation. Determination of the functional effect of a compound on metastatic breast cancer or metastatic lung cancer can also be performed using metastatic breast cancer or metastatic lung cancer assays known to those of skill in the art such as an in vitro assays, e.g., cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of metastatic breast cancer or metastatic lung cancer cells. The functional effects can be evaluated by many means known to those skilled in the art, e.g., microscopy for quantitative or qualitative measures of alterations in morphological features, measurement of changes in RNA or protein levels for metastatic breast cancer or metastatic lung cancer-associated sequences, measurement of RNA stability, identification of downstream or reporter gene expression (CAT, luciferase, β-gal, GFP and the like), e.g., via chemiluminescence, fluorescence, calorimetric reactions, antibody binding, inducible markers, and ligand binding assays.

“Inhibitors”, “activators”, and “modulators” of metastatic breast cancer or metastatic lung cancer polynucleotide and polypeptide sequences are used to refer to activating, inhibitory, or modulating molecules or compounds identified using in vitro and in vivo assays of metastatic breast cancer or metastatic lung cancer polynucleotide and polypeptide sequences of the invention. Inhibitors are compounds that, e.g., bind to, partially or totally block activity, decrease, prevent, delay activation, inactivate, desensitize, or down regulate the activity or expression of metastatic breast cancer or metastatic lung cancer proteins of the invention, e.g., antagonists. Antisense nucleic acids may seem to inhibit expression and subsequent function of the protein. “Activators” are compounds that increase, open, activate, facilitate, enhance activation, sensitize, agonize, or up regulate metastatic breast cancer or metastatic lung cancer protein activity. Inhibitors, activators, or modulators also include genetically modified versions of metastatic breast cancer or metastatic lung cancer proteins, e.g., versions with altered activity, as well as naturally occurring and synthetic ligands, antagonists, agonists, antibodies, small chemical molecules and the like. Such assays for inhibitors and activators include, e.g., expressing the metastatic breast cancer or metastatic lung cancer protein in vitro, in cells, or cell membranes, applying putative modulator compounds, and then determining the functional effects on activity, as described above. Activators and inhibitors of metastatic breast cancer or metastatic lung cancer can also be identified by incubating metastatic breast cancer or metastatic lung cancer cells with the test compound and determining increases or decreases in the expression of 1 or more metastatic breast cancer or metastatic lung cancer proteins, e.g., 1, 2, 3, 4, 5, 10, 15, 20, 25, 30, 40, 50 or more metastatic breast cancer or metastatic lung cancer proteins, such as metastatic breast cancer or metastatic lung cancer proteins encoded by the sequences set out in Tables 1-12.

Samples or assays comprising metastatic breast cancer or metastatic lung cancer proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of a metastatic breast cancer or metastatic lung cancer polypeptide is achieved when the activity value relative to the control (untreated with activators) is 110%, more preferably 150%, more preferably 200-500% (i.e., two to five fold higher relative to the control), more preferably 1000-3000% higher.

The phrase “changes in cell growth” refers to any change in cell growth and proliferation characteristics in vitro or in vivo, such as formation of foci, anchorage independence, semi-solid or soft agar growth, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. See, e.g., Freshney, Culture of Animal Cells a Manual of Basic Technique pp. 231-241 (3^{rd} ed. 1994).

“Tumor cell” refers to precancerous, cancerous, and normal cells in a tumor. “Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy (see, Freshney, Culture of Animal Cells a Manual ofBasic Technique (3^{rd} ed. 1994)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab′)₂, a dimer of Fab which itself is a light chain joined to V_{H}-C_{H}1 by a disulfide bond. The F(ab′)₂ may be reduced under mild conditions to break the disulfide linkage in the hinge region, thereby converting the F(ab′)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which, e.g, (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

### Identification of Metastatic Breast Cancer or Metastatic Lung Cancer-associated Sequences

In one aspect, the expression levels of genes are determined in different patient samples for which diagnosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is characteristic of the state of the cell. That is, normal tissue may be distinguished from cancerous or metastatic cancerous tissue, or metastatic cancerous tissue can be compared with tissue from surviving cancer patients. By comparing expression profiles of tissue in known different metastatic breast cancer or metastatic lung cancer states, information regarding which genes are important (including both up- and down-regulation of genes) in each of these states is obtained.

The identification of sequences that are differentially expressed in metastatic breast cancer or metastatic lung cancer versus non-metastatic breast cancer or non-metastatic lung cancer tissue allows the use of this information in a number of ways. For example, a particular treatment regime may be evaluated: does a chemotherapeutic drug act to down-regulate metastatic breast cancer or metastatic lung cancer, and thus tumor growth or recurrence, in a particular patient. Similarly, diagnosis and treatment outcomes may be done or confirmed by comparing patient samples with the known expression profiles. Metastatic tissue can also be analyzed to determine the stage of metastatic breast cancer or metastatic lung cancer in the tissue. Furthermore, these gene expression profiles (or individual genes) allow screening of drug candidates with an eye to mimicking or altering a particular expression profile; e.g., screening can be done for drugs that suppress the metastatic breast cancer or metastatic lung cancer expression profile. This may be done by making biochips comprising sets of the important metastatic breast cancer or metastatic lung cancer genes, which can then be used in these screens. PCR methods may be applied with selected primer pairs, and analysis may be of RNA or of genomic sequences. These methods can also be done on the protein basis; that is, protein expression levels of the metastatic breast cancer or metastatic lung cancer proteins can be evaluated for diagnostic purposes or to screen candidate agents. In addition, the metastatic breast cancer or metastatic lung cancer nucleic acid sequences can be administered for gene therapy purposes, including the administration of antisense nucleic acids, or the metastatic breast cancer or metastatic lung cancer proteins (including antibodies and other modulators thereof) administered as therapeutic drugs or as protein or DNA vaccines.

Thus the present invention provides nucleic acid and protein sequences that are differentially expressed in metastatic breast cancer or metastatic lung cancer, herein termed “metastatic breast cancer or metastatic lung cancer sequences.” As outlined below, metastatic breast cancer or metastatic lung cancer sequences include those that are up- regulated (i.e., expressed at a higher level) in metastatic breast cancer or metastatic lung cancer, as well as those that are down-regulated (i.e., expressed at a lower level). In a preferred embodiment, the metastatic breast cancer or metastatic lung cancer sequences are from humans; however, as will be appreciated by those in the art, metastatic breast cancer or metastatic lung cancer sequences from other organisms may be useful in animal models of disease and drug evaluation; thus, other metastatic breast cancer or metastatic lung cancer sequences are provided, from vertebrates, including mammals, including rodents (rats, mice, hamsters, guinea pigs, etc.), primates, farm animals (including sheep, goats, pigs, cows, horses, etc.) and pets (dogs, cats, etc.). Metastatic breast cancer or metastatic lung cancer sequences from other organisms may be obtained using the techniques outlined below.

Metastatic breast cancer or metastatic lung cancer sequences can include both nucleic acid and amino acid sequences. As will be appreciated by those in the art and is more fully outlined below, metastatic breast cancer or metastatic lung cancer nucleic acid sequences are useful in a variety of applications, including diagnostic applications, which will detect naturally occurring nucleic acids, as well as screening applications; e.g., biochips comprising nucleic acid probes or PCR microtiter plates with selected probes to the metastatic breast cancer or metastatic lung cancer sequences can be generated.

A metastatic breast cancer or metastatic lung cancer sequence can be initially identified by substantial nucleic acid and/or amino acid sequence homology to the metastatic breast cancer or metastatic lung cancer sequences outlined herein. Such homology can be based upon the overall nucleic acid or amino acid sequence, and is generally determined as outlined below, using either homology programs or hybridization conditions.

For identifying metastatic breast cancer or metastatic lung cancer-associated sequences, the metastatic breast cancer or metastatic lung cancer screen typically includes comparing genes identified in different tissues, e.g., normal and cancerous tissues, or tumor tissue samples from patients who have metastatic disease vs. non metastatic tissue, or tumor tissue samples from patients who have been diagnosed cancer but have survived vs. metastatic tissue. Other suitable tissue comparisons include comparing metastatic breast cancer or metastatic lung cancer samples with metastatic cancer samples from other cancers, such as gastrointestinal cancers, prostate, ovarian, etc. Samples of, e.g., breast cancer survivor tissue and tissue undergoing metastasis are applied to biochips comprising nucleic acid probes. The samples are first microdissected, if applicable, and treated as is known in the art for the preparation of mRNA. Suitable biochips are commercially available, e.g., GENECHIP® (DNA microarray) technology from Affymetrix, Inc. (Santa Clara, Calif.). Gene expression profiles as described herein are generated and the data analyzed.

In one embodiment, the genes showing changes in expression as between normal and disease states are compared to genes expressed in other normal tissues, preferably normal colon, but also including, and not limited to lung, heart, brain, liver, breast, kidney, muscle, prostate, small intestine, large intestine, spleen, bone and placenta. In a preferred embodiment, those genes identified during the metastatic breast cancer or metastatic lung cancer screen that are expressed in significant amounts in other tissues are removed from the profile, although in some embodiments, this is not necessary. That is, when screening for drugs, it is usually preferable that the target be disease specific, to minimize possible side effects.

In a preferred embodiment, metastatic breast cancer or metastatic lung cancer sequences are those that are up-regulated in metastatic breast cancer or metastatic lung cancer; that is, the expression of these genes is higher in the metastatic tissue as compared to non-metastatic cancerous tissue or normal tissue (see, e.g., Tables 1-3, 6-8, 11-12). “Up-regulation” as used herein means, when the ratio is presented as a number greater than one, that the ratio is greater than one, preferably 1.5 or greater, more preferably 2.0 or greater. All UniGene cluster identification numbers and accession numbers herein are for the GenBank sequence database and the sequences of the accession numbers are hereby expressly incorporated by reference. GenBank is known in the art, see, e.g., Benson, D A, et al., Nucleic Acids Research 26:1-7 (1998) and. Sequences are also available in other databases, e.g., European Molecular Biology Laboratory (EMBL) and DNA Database of Japan (DDBJ).

In another preferred embodiment, metastatic breast cancer or metastatic lung cancer sequences are those that are down-regulated in the metastatic breast cancer or metastatic lung cancer; that is, the expression of these genes is lower in metastatic tissue as compared to non-metastatic cancerous tissue or normal tissue (see, e.g., Tables 4-5 and 9-10). “Down-regulation” as used herein means, when the ratio is presented as a number greater than one, that the ratio is greater than one, preferably 1.5 or greater, more preferably 2.0 or greater, or, when the ratio is presented as a number less than one, that the ratio is less than one, preferably 0.5 or less, more preferably 0.25 or less.

Informatics

The ability to identify genes that are over or under expressed in metastatic breast cancer or metastatic lung cancer can additionally provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients with metastatic breast cancer or metastatic lung cancer. Or as another example, subcellular toxicological information can be generated to better direct drug structure and activity correlation (see Anderson, Pharmaceutical Proteomics: Targets, Mechanism, and Function, paper presented at the IBC Proteomics conference, Coronado, Calif. (June 11-12, 1998)). Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases can be assembled for assay data acquired using an assay of the invention.

The compositions and methods for identifying and/or quantitating the relative and/or absolute abundance of a variety of molecular and macromolecular species from a biological sample undergoing metastatic breast cancer or metastatic lung cancer, i.e., the identification of metastatic breast cancer or metastatic lung cancer-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein finction hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioinformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinformatics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for metastatic breast cancer or metastatic lung cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 1OBaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

### Characteristics of Metastatic Breast Cancer or Metastatic Lung Cancer-associated Proteins

Metastatic breast cancer or metastatic lung cancer proteins of the present invention may be classified as secreted proteins, transmembrane proteins or intracellular proteins. In one embodiment, the metastatic breast cancer or metastatic lung cancer protein is an intracellular protein. Intracellular proteins may be found in the cytoplasm and/or in the nucleus and/or in the organelles. Proteins containing one or more transmembrane domains that exclusively reside in organelles are also considered intracellular proteins. Intracellular proteins are involved in all aspects of cellular function and replication (including, e.g., signaling pathways); aberrant expression of such proteins often results in unregulated or disregulated cellular processes (see, e.g., Molecular Biology of the Cell (Alberts, ed., 3rd ed., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden (see, e.g., Bateman et al., Nuc. Acids Res. 28:263-266 (2000); Sonnhammer et al., Proteins 28:405-420 (1997); Bateman et al., Nuc. Acids Res. 27:260-262 (1999); and Sonnhammer et al., Nuc. Acids Res. 26:320-322-(1998)).

In another embodiment, the metastatic breast cancer or metastatic lung cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels, pumps, and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted (see, e.g. PSORT web site).

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, hormones, neurotrophic factors and the like. Extracellular domains also bind to cell-associated molecules. In this respect, they mediate cell-cell interactions. Cell-associated ligands can be tethered to the cell, e.g., via a glycosylphosphatidylinositol (GPI) anchor, or may themselves be transmembrane proteins. Extracellular domains also associate with the extracellular matrix and contribute to the maintenance of the cell structure.

Metastatic breast cancer or metastatic lung cancer proteins that are transmembrane are particularly preferred in the present invention as they are readily accessible targets for extracellular immunotherapeutics, as are described herein. In addition, as outlined below, transmembrane proteins can be also useful in imaging modalities. Antibodies may be used to label such readily accessible proteins in situ or in histological analysis. Alternatively, antibodies can also label intracellular proteins, in which case analytical samples are typically permeablized to provide access to intracellular proteins.

It will also be appreciated by those in the art that a transmembrane protein can be made soluble by removing transmembrane sequences, e.g., through recombinant methods. Furthermore, transmembrane proteins that have been made soluble can be made to be secreted through recombinant means by adding an appropriate signal sequence.

In another embodiment, the metastatic breast cancer or metastatic lung cancer proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they often serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Metastatic breast cancer or metastatic lung cancer proteins that are secreted proteins are particularly preferred in the present invention as they serve as good targets for diagnostic markers, e.g., for blood, plasma, serum, or stool tests.

### Use of metastatic Breast Cancer or Metastatic Lung Cancer Nucleic Acids

As described above, metastatic breast cancer or metastatic lung cancer sequence is initially identified by substantial nucleic acid and/or amino acid sequence homology or linkage to the metastatic breast cancer or metastatic lung cancer sequences outlined herein. Such homology can be based upon the overall nucleic acid or amino acid sequence, and is generally determined as outlined below, using either homology programs or hybridization conditions. Typically, linked sequences on a mRNA are found on the same molecule.

The metastatic breast cancer or metastatic lung cancer nucleic acid sequences of the invention, e.g., the sequences in Tables 1-12, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the metastatic breast cancer or metastatic lung cancer genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene.

Once the metastatic breast cancer or metastatic lung cancer nucleic acid is identified, it can be cloned and, if necessary, its constituent parts recombined to form the entire metastatic breast cancer or metastatic lung cancer nucleic acid coding regions or the entire mRNA sequence. Once isolated from its natural source, e.g., contained within a plasmid or other vector or excised therefrom as a linear nucleic acid segment, the recombinant metastatic breast cancer or metastatic lung cancer nucleic acid can be further-used as a probe to identify and isolate other metastatic breast cancer or metastatic lung cancer nucleic acids, e.g., extended coding regions. It can also be used as a “precursor” nucleic acid to make modified or variant metastatic breast cancer or metastatic lung cancer nucleic acids and proteins.

The metastatic breast cancer or metastatic lung cancer nucleic acids of the present invention are used in several ways. In a first embodiment, nucleic acid probes to the metastatic breast cancer or metastatic lung cancer nucleic acids are made and attached to biochips to be used in screening and diagnostic methods, as outlined below, or for administration, e.g., for gene therapy, vaccine, and/or antisense applications. Alternatively, the metastatic breast cancer or metastatic lung cancer nucleic acids that include coding regions of metastatic breast cancer or metastatic lung cancer proteins can be put into expression vectors for the expression of metastatic breast cancer or metastatic lung cancer proteins, again for screening purposes or for administration to a patient.

In a preferred embodiment, nucleic acid probes to metastatic breast cancer or metastatic lung cancer nucleic acids (both the nucleic acid sequences outlined in the figures and/or the complements thereof) are made. The nucleic acid probes attached to the biochip are designed to be substantially complementary to the metastatic breast cancer or metastatic lung cancer nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under appropriate reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally complements of ORFs or whole genes are not used. In some embodiments, nucleic acids of lengths up to hundreds of bases can be used.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is typically meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to a biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, e.g., using linkers as are known in the art; e.g., homo-or hetero-bifunctional linkers as are well known (see 1994 Pierce Chemical Company catalog, technical section on cross-linkers, pages 155-200). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the GENECHIP® (DNA microarray) technology from Affymetrix, Inc. (Santa Clara, Calif.).

Often, amplification-based assays are performed to measure the expression level of metastatic breast cancer or metastatic lung cancer-associated sequences. These assays are typically performed in conjunction with reverse transcription. In such assays, a metastatic breast cancer or metastatic lung cancer-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of metastatic breast cancer or metastatic lung cancer-associated RNA. Methods of quantitative amplification are well known to those of skill in the art. Detailed protocols for quantitative PCR are provided, e.g., in Innis et al., PCR Protocols, A Guide to Methods and Applications (1990).

In some embodiments, a TAQMAN® (PCR reagent kit) based assay is used to measure expression. TAQMAN® (PCR reagent kit) based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AMPLITAQO (PCR enzyme reagent), results in the cleavage of the TAQMAN® (PCR reagent kit) probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

### Expression of Metastatic breast cancer or metastatic lung cancer proteins from nucleic acids

In a preferred embodiment, metastatic breast cancer or metastatic lung cancer nucleic acids, e.g., encoding metastatic breast cancer or metastatic lung cancer proteins, are used to make a variety of expression vectors to express metastatic breast cancer or metastatic lung cancer proteins which can then be used in screening assays, as described below. Expression vectors and recombinant DNA technology are well known to those of skill in the art (see, e.g., Ausubel, supra, and Gene Expression Systems (Fernandez & Hoeffler, eds, 1999)) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the metastatic breast cancer or metastatic lung cancer protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the metastatic breast cancer or metastatic lung cancer protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, e.g., in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra).

In addition, in a preferred embodiment, the expression vector contains a selectable marker gene to allow the selection of transformed host cells. Selection genes are well known in the art and will vary with the host cell used.

The metastatic breast cancer or metastatic lung cancer proteins of the present invention are produced by culturing a host cell transformed with an expression vector containing nucleic acid encoding a metastatic breast cancer or metastatic lung cancer protein, under the appropriate conditions to induce or cause expression of the metastatic breast cancer or metastatic lung cancer protein. Conditions appropriate for metastatic breast cancer or metastatic lung cancer protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THPl cells (a macrophage cell line) and various other human cells and cell lines.

In a preferred embodiment, the metastatic breast cancer or metastatic lung cancer proteins are expressed in mammalian cells. Mammalian expression systems are also known in the art, and include retroviral and adenoviral systems. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, metastatic breast cancer or metastatic lung cancer proteins are expressed in bacterial systems. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the metastatic breast cancer or metastatic lung cancer protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez & Hoeffler, supra). The bacterial expression vectors are transformed into bacterial host cells using techniques well known in the art, such as calcium chloride treatment, electroporation, and others.

In one embodiment, metastatic breast cancer or metastatic lung cancer proteins are produced in insect cells. Expression vectors for the transformation of insect cells, and in particular, baculovirus-based expression vectors, are well known in the art.

In a preferred embodiment, metastatic breast cancer or metastatic lung cancer protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The metastatic breast cancer or metastatic lung cancer protein may also be made as a fusion protein, using techniques well known in the art. Thus, e.g., for the creation of monoclonal antibodies, if the desired epitope is small, the metastatic breast cancer or metastatic lung cancer protein may be fused to a carrier protein to form an immunogen. Alternatively, the metastatic breast cancer or metastatic lung cancer protein may be made as a fusion protein to increase expression for affinity purification purposes, or for other reasons. For example, when the metastatic breast cancer or metastatic lung cancer protein is a metastatic breast cancer or metastatic lung cancer peptide, the nucleic acid encoding the peptide may be linked to other nucleic acid for expression purposes.

In a preferred embodiment, the metastatic breast cancer or metastatic lung cancer protein is purified or isolated after expression. Metastatic breast cancer or metastatic lung cancer proteins may be isolated or purified in a variety of appropriate ways. Standard purification methods include electrophoretic, molecular, immunological and chromatographic techniques, including ion exchange, hydrophobic, affinity, and reverse-phase HPLC chromatography, and chromatofocusing. For example, the metastatic breast cancer or metastatic lung cancer protein may be purified using a standard anti-metastatic breast cancer or metastatic lung cancer protein antibody colurn. Ultrafiltration and diafiltration techniques, in conjunction with protein concentration, are also useful. For general guidance in suitable purification techniques, see Scopes, Protein Purification (1982). The degree of purification necessary will vary depending on the use of the metastatic breast cancer or metastatic lung cancer protein. In some instances no purification will be necessary.

Once expressed and purified if necessary, the metastatic breast cancer or metastatic lung cancer proteins and nucleic acids are useful in a number of applications. They may be used as immunoselection reagents, as vaccine reagents, as screening agents, etc.

### Variants of metastatic breast cancer or metastatic lung cancer proteins

In one embodiment, the metastatic breast cancer or metastatic lung cancer proteins are derivative or variant metastatic breast cancer or metastatic lung cancer proteins as compared to the wild-type sequence. That is, as outlined more fully below, the derivative metastatic breast cancer or metastatic lung cancer peptide will often contain at least one amino acid substitution, deletion or insertion, with amino acid substitutions being particularly preferred. The amino acid substitution, insertion or deletion may occur at a particular residue within the metastatic breast cancer or metastatic lung cancer peptide.

Also included within one embodiment of metastatic breast cancer or metastatic lung cancer proteins of the present invention are amino acid sequence variants. These variants typically fall into one or more of three classes: substitutional, insertional or deletional variants. These variants ordinarily are prepared by site specific mutagenesis of nucleotides in the DNA encoding the metastatic breast cancer or metastatic lung cancer protein, using cassette or PCR mutagenesis or other techniques, to produce DNA encoding the variant, and thereafter expressing the DNA in recombinant cell culture as outlined above. However, variant metastatic breast cancer or metastatic lung cancer protein fragments having up to about 100-150 residues may be prepared by in vitro synthesis. Amino acid sequence variants are characterized by the predetermined nature of the variation, a feature that sets them apart from naturally occurring allelic or interspecies variation of the metastatic breast cancer or metastatic lung cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is often predetermined, the mutation per se need not be predetermined. For example, in order to optimize the performance of a mutation at a given site, random mutagenesis may be conducted at the target codon or region and the expressed metastatic breast cancer or metastatic lung cancer variants screened for the optimal combination of desired activity. Techniques exist for making substitution mutations at predetermined sites in DNA having a known sequence, e.g., M13 primer mutagenesis and PCR mutagenesis. Screening of the mutants is done using assays of metastatic breast cancer or metastatic lung cancer protein activities.

Amino acid substitutions are typically of single residues; insertions usually will be on the order of from about 1 to 20 amino acids, although considerably larger insertions may be occasionally tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. Larger changes may be tolerated in certain circumstances. When small alterations in the characteristics of a metastatic breast cancer or metastatic lung cancer protein are desired, substitutions are generally made in accordance with the amino acid substitution chart provided in the definition section.

Variants typically exhibit the same qualitative biological activity and will elicit the same immune response as the naturally-occurring analog, although variants also are selected to modify the characteristics of the metastatic breast cancer or metastatic lung cancer proteins as needed. Alternatively, the variant may be designed or reorganized such that the biological activity of the metastatic breast cancer or metastatic lung cancer protein is altered. For example, glycosylation sites may be altered or removed.

Covalent modifications of metastatic breast cancer or metastatic lung cancer polypeptides are included within the scope of this invention. One type of covalent modification includes reacting targeted amino acid residues of a metastatic breast cancer or metastatic lung cancer polypeptide with an organic derivatizing agent that is capable of reacting with selected side chains or the N-or C-terminal residues of a metastatic breast cancer or metastatic lung cancer polypeptide. Derivatization with bifunctional agents is useful, for instance, for crosslinking metastatic breast cancer or metastatic lung cancer polypeptides to a water-insoluble support matrix or surface for use in the method for purifying anti-metastatic breast cancer or metastatic lung cancer polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, e.g., esters with 4-azidosalicylic acid, homobifunctional imidoesters, including disuccinimidyl esters such as 3,3′-dithiobis(succinimidylpropionate), bifinctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the γ-amino groups of lysine, arginine, and histidine side chains (Creighton, Proteins: Structure and Molecular Properties, pp. 79-86 (1983)), acetylation of the N-terminal amine, and amidation of any C-terminal carboxyl group.

Another type of covalent modification of the metastatic breast cancer or metastatic lung cancer polypeptide encompassed by this invention is an altered native glycosylation pattern of the polypeptide. “Altering the native glycosylation pattern” is intended herein to mean adding to or deleting one or more carbohydrate moieties of a native sequence metastatic breast cancer or metastatic lung cancer polypeptide. Glycosylation patterns can be altered in many ways. For example the use of different cell types to express metastatic breast cancer or metastatic lung cancer-associated sequences can result in different glycosylation patterns.

Addition of glycosylation sites to metastatic breast cancer or metastatic lung cancer polypeptides may also be accomplished by altering the amino acid sequence thereof. The alteration may be made, e.g., by the addition of, or substitution by, one or more serine or threonine residues to the native sequence metastatic breast cancer or metastatic lung cancer polypeptide (for O-linked glycosylation sites). The metastatic breast cancer or metastatic lung cancer amino acid sequence may optionally be altered through changes at the DNA level, particularly by mutating the DNA encoding the metastatic breast cancer or metastatic lung cancer polypeptide at preselected bases such that codons are generated that will translate into the desired amino acids.

Another means of increasing the number of carbohydrate moieties on the metastatic breast cancer or metastatic lung cancer polypeptide is by chemical or enzymatic coupling of glycosides to the polypeptide. Such methods are described in the art, e.g., in WO 87/05330, and in Aplin & Wriston, CRC Crit. Rev. Biochem., pp. 259-306 (1981).

Removal of carbohydrate moieties present on the metastatic breast cancer or metastatic lung cancer polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of metastatic breast cancer or metastatic lung cancer comprises linking the metastatic breast cancer or metastatic lung cancer polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337, each of which is hereby incorporated by reference herein.

Metastatic breast cancer or metastatic lung cancer polypeptides of the present invention may also be modified in a way to form chimeric molecules comprising a metastatic breast cancer or metastatic lung cancer polypeptide fused to another, heterologous polypeptide or amino acid sequence. In one embodiment, such a chimeric molecule comprises a fusion of a metastatic breast cancer or metastatic lung cancer polypeptide with a tag polypeptide which provides an epitope to which an anti-tag antibody can selectively bind. The epitope tag is generally placed at the amino-or carboxyl-terminus of the metastatic breast cancer or metastatic lung cancer polypeptide. The presence of such epitope-tagged forms of a metastatic breast cancer or metastatic lung cancer polypeptide can be detected using an antibody against the tag polypeptide. Also, provision of the epitope tag enables the metastatic breast cancer or metastatic lung cancer polypeptide to be readily purified by affinity purification using an anti-tag antibody or another type of affinity matrix that binds to the epitope tag. In an alternative embodiment, the chimeric molecule may comprise a fusion of a metastatic breast cancer or metastatic lung cancer polypeptide with an immunoglobulin or a particular region of an immunoglobulin. For a bivalent form of the chimeric molecule, such a fusion could be to the Fc region of an IgG molecule.

Various tag polypeptides and their respective antibodies are well known and examples include poly-histidine (poly-his) or poly-histidine-glycine (poly-his-gly) tags; HIS6 and metal chelation tags, the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J Biol. Chem. 266:15163-15166 (1991)); and the T7 gene 10 protein peptide tag (Lutz-Freyermuth et al., Proc. Natl. Acad. Sci. USA 87:6393-6397 (1990)).

Also included are other metastatic breast cancer or metastatic lung cancer proteins of the metastatic breast cancer or metastatic lung cancer family, and metastatic breast cancer or metastatic lung cancer proteins from other organisms, which are cloned and expressed as outlined below. Thus, probe or degenerate polymerase chain reaction (PCR) primer sequences may be used to find other related metastatic breast cancer or metastatic lung cancer proteins from primates or other organisms. As will be appreciated by those in the art, particularly useful probe and/or PCR primer sequences include unique areas of the metastatic breast cancer or metastatic lung cancer nucleic acid sequence. As is generally known in the art, preferred PCR primers are from about 15 to about 35 nucleotides in length, with from about 20 to about 30 being preferred, and may contain inosine as needed. PCR reaction conditions are well known in the art (e.g., Innis, PCR Protocols, supra).

### Antibodies to Metastatic Breast Cancer or Metastatic Lung Cancer Proteins

In a preferred embodiment, when a metastatic breast cancer or metastatic lung cancer protein is to be used to generate antibodies, e.g., for immunotherapy or immunodiagnosis, the metastatic breast cancer or metastatic lung cancer protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller metastatic breast cancer or metastatic lung cancer protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are well known (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of Tables 1-12 or fragment thereof or a fusion protein thereof. It may be useful to conjugate the immunizing agent to a protein known to be immunogenic in the mammal being immunized. Immunogenic proteins include, e.g., keyhole limpet hemocyanin, serum albumin, bovine thyroglobulin, and soybean trypsin inhibitor. Adjuvants include, e.g., Freund's complete adjuvant and MPL-TDM adjuvant (monophosphoryl Lipid A, synthetic trehalose dicorynomycolate). The immunization protocol may be selected by one skilled in the art.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-12, or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and primate origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are typically monoclonal, preferably human or humanized, antibodies that have binding specificities for at least two different antigens or that have binding specificities for two epitopes on the same antigen. In one embodiment, one of the binding specificities is for a protein encoded by a nucleic acid of Tables 1-12 or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to metastatic breast cancer or metastatic lung cancer protein are capable of reducing or eliminating a biological function of a metastatic breast cancer or metastatic lung cancer protein, as is described below. That is, the addition of anti-metastatic breast cancer or metastatic lung cancer protein antibodies (either polyclonal or preferably monoclonal) to metastatic breast cancer or metastatic lung cancer tissue (or cells containing metastatic breast cancer or metastatic lung cancer) may reduce or eliminate the metastatic breast cancer or metastatic lung cancer. Generally, at least a 25% decrease in activity, growth, size or the like is preferred, with at least about 50% being particularly preferred and about a 95-100% decrease being especially preferred.

In a preferred embodiment the antibodies to the metastatic breast cancer or metastatic lung cancer proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non- human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988);. and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567, which is hereby incorporated by reference herein.), wherein substantially less than an intact human variable domain has been substituted by the corresponding sequence from a non-human species.

Human-like antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boemer et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in virtually all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, each of which is hereby incorporated by reference herein, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

By immunotherapy is meant treatment of metastatic breast cancer or metastatic lung cancer with an antibody raised against a metastatic breast cancer or metastatic lung cancer proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. The antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the metastatic breast cancer or metastatic lung cancer proteins against which antibodies are raised are secreted proteins as described above. Without being bound by theory, antibodies used for treatment, bind and prevent the secreted protein from binding to its receptor, thereby inactivating the secreted metastatic breast cancer or metastatic lung cancer protein.

In another preferred embodiment, the metastatic breast cancer or metastatic lung cancer protein to which antibodies are raised is a transmembrane protein. Without being bound by theory, antibodies used for this treatment typically bind the extracellular domain of the metastatic breast cancer or metastatic lung cancer protein and prevent it from binding to other proteins, such as circulating ligands or cell-associated molecules. The antibody may cause down-regulation of the transmembrane metastatic breast cancer or metastatic lung cancer protein. The antibody may be a competitive, non-competitive or uncompetitive inhibitor of protein binding to the extracellular domain of the metastatic breast cancer or metastatic lung cancer protein. The antibody may be an antagonist of the metastatic breast cancer or metastatic lung cancer protein or may prevent activation of the transmembrane metastatic breast cancer or metastatic lung cancer protein. In some embodiments, when the antibody prevents the binding of other molecules to the metastatic breast cancer or metastatic lung cancer protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, metastatic breast cancer or metastatic lung cancer is treated by administering to a patient antibodies directed against the transmembrane metastatic breast cancer or metastatic lung cancer protein. Antibody-labeling may activate a co-toxin, localize a toxin payload, or otherwise provide means to locally ablate cells.

In another preferred embodiment, the antibody is conjugated to an effector moiety. The effector moiety can be any number of molecules, including labeling moieties such as radioactive labels or fluorescent labels, or can be a therapeutic moiety. In one aspect the therapeutic moiety is a small molecule that modulates the activity of the metastatic breast cancer or metastatic lung cancer protein. In another aspect the therapeutic moiety modulates the activity of molecules associated with or in close proximity to the metastatic breast cancer or metastatic lung cancer protein. The therapeutic moiety may inhibit enzymatic activity such as protease or collagenase activity associated with metastatic breast cancer or metastatic lung cancer.

In a preferred embodiment, the therapeutic moiety can also be a cytotoxic agent. In this method, targeting the cytotoxic agent to metastatic breast cancer or metastatic lung cancer tissue or cells. results in a reduction in the number of afflicted cells, thereby reducing symptoms associated with metastatic breast cancer or metastatic lung cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against metastatic breast cancer or metastatic lung cancer proteins, or binding of a radionuclide to a chelating agent that has been covalently attached to the antibody. Targeting the therapeutic moiety to transmembrane metastatic breast cancer or metastatic lung cancer proteins not only serves to increase the local concentration of therapeutic moiety in the metastatic breast cancer or metastatic lung cancer afflicted area, but also serves to reduce deleterious side effects that may be associated with the therapeutic moiety.

In another preferred embodiment, the metastatic breast cancer or metastatic lung cancer protein against which the antibodies are raised is an intracellular protein. In this case, the antibody may be conjugated to a protein or other entity which facilitates entry into the cell. In one case, the antibody enters the cell by endocytosis. In another embodiment, a nucleic acid encoding the antibody is administered to the individual or cell. Moreover, wherein the metastatic breast cancer or metastatic lung cancer protein can be targeted within a cell, i.e., the nucleus, an antibody thereto contains a signal for that target localization, i.e., a nuclear localization signal.

The metastatic breast cancer or metastatic lung cancer antibodies of the invention specifically bind to metastatic breast cancer or metastatic lung cancer proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

### Detection of Metastatic Breast Cancer or Metastatic Lung Cancer Sequence for Diagnostic and Therapeutic Applications

In one aspect, the RNA expression levels of genes are determined for different cellular states in the metastatic breast cancer or metastatic lung cancer phenotype. Expression levels of genes in normal tissue (i.e., not undergoing metastatic breast cancer or metastatic lung cancer) and in metastatic breast cancer or metastatic lung cancer tissue (and in some cases, for varying severities of metastatic breast cancer or metastatic lung cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus metastatic breast cancer or metastatic lung cancer tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of GENECHIP® (DNA microarray) expression arrays from Affymetrix, Inc. (Santa Clara, Calif.), as described in Lockhart et al., Nature Biotechnology 14:1675-1680 (1996), hereby expressly incorporated by reference. Other techniques include, but are not limited to, quantitative reverse transcriptase PCR, northern analysis and RNase protection. As outlined above, preferably the change in expression (i.e., upregulation or downregulation) is typically at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the metastatic breast cancer or metastatic lung cancer protein and standard immunoassays (ELISAs, etc.) or other techniques, including mass spectroscopy assays, 2D gel electrophoresis assays, etc. Proteins corresponding to metastatic breast cancer or metastatic lung cancer genes, i.e., those identified as being important in a metastatic breast cancer or metastatic lung cancer phenotype, can be evaluated in a metastatic breast cancer or metastatic lung cancer diagnostic test.

In a preferred embodiment, gene expression monitoring is performed simultaneously on a number of genes.

The metastatic breast cancer or metastatic lung cancer nucleic acid probes may be attached to biochips as outlined herein for the detection and quantification of metastatic breast cancer or metastatic lung cancer sequences in a particular cell. The assays are further described below in the example. PCR techniques can be used to provide greater sensitivity. Multiple protein expression monitoring can be performed as well. Similarly, these assays may be performed on an individual basis as well.

In a preferred embodiment nucleic acids encoding the metastatic breast cancer or metastatic lung cancer protein are detected. Although DNA or RNA encoding the metastatic breast cancer or metastatic lung cancer protein may be detected, of particular interest are methods wherein an mRNA encoding a metastatic breast cancer or metastatic lung cancer protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a metastatic breast cancer or metastatic lung cancer protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The metastatic breast cancer or metastatic lung cancer proteins, antibodies, nucleic acids, modified proteins and cells containing metastatic breast cancer or metastatic lung cancer sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, metastatic breast cancer or metastatic lung cancer proteins, including intracellular, transmembrane or secreted proteins, find use as markers of metastatic breast cancer or metastatic lung cancer. Detection of these proteins in putative metastatic breast cancer or metastatic lung cancer tissue allows for detection or diagnosis of metastatic breast cancer or metastatic lung cancer. In one embodiment, antibodies are used to detect metastatic breast cancer or metastatic lung cancer proteins. A preferred method separates proteins from a sample by electrophoresis on a gel (typically a denaturing and reducing protein gel, but may be another type of gel, including isoelectric focusing gels and the like). Following separation of proteins, the metastatic breast cancer or metastatic lung cancer protein is detected, e.g., by immunoblotting with antibodies raised against the metastatic breast cancer or metastatic lung cancer protein. Methods of immunoblotting are well known to those of ordinary skill in the art.

In another preferred method, antibodies to the metastatic breast cancer or metastatic lung cancer protein find use in in situ imaging techniques, e.g., in histology (e.g., Methods in Cell Biology: Antibodies in Cell Biology, volume 37 (Asai, ed. 1993)). In this method cells are contacted with from one to many antibodies to the metastatic breast cancer or metastatic lung cancer protein(s). Following washing to remove non-specific antibody binding, the presence of the antibody or antibodies is detected. In one embodiment the antibody is detected by incubating with a secondary antibody that contains a detectable label, e.g., multicolor fluorescence or confocal imaging. In another method the primary antibody to the metastatic breast cancer or metastatic lung cancer protein(s) contains a detectable label, e.g., an enzyme marker that can act on a substrate. In another preferred embodiment each one of multiple primary antibodies contains a distinct and detectable label. This method finds particular use in simultaneous screening for a plurality of metastatic breast cancer or metastatic lung cancer proteins. Many other histological imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing metastatic breast cancer or metastatic lung cancer from blood, serum, plasma, stool, and other samples. Such samples, therefore, are useful as samples to be probed or tested for the presence of metastatic breast cancer or metastatic lung cancer proteins. Antibodies can be used to detect a metastatic breast cancer or metastatic lung cancer protein by previously described immunoassay techniques including ELISA, immunoblotting (western blotting), immunoprecipitation, BIACORE technology and the like. Conversely, the presence of antibodies may indicate an immune response against an endogenous metastatic breast cancer or metastatic lung cancer protein or vaccine.

In a preferred embodiment, in situ hybridization of labeled metastatic breast cancer or metastatic lung cancer nucleic acid probes to tissue arrays is done. For example, arrays of tissue samples, including metastatic breast cancer or metastatic lung cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the metastatic breast cancer or metastatic lung cancer proteins, antibodies, nucleic acids, modified proteins and cells containing metastatic breast cancer or metastatic lung cancer sequences are used in prognosis assays. As above, gene expression profiles can be generated that correlate to metastatic breast cancer or metastatic lung cancer, in terms of long term prognosis. Again, this may be done on either a protein or gene level, with the use of genes being preferred. As above, metastatic breast cancer or metastatic lung cancer probes may be attached to biochips for the detection and quantification of metastatic breast cancer or metastatic lung cancer sequences in a tissue or patient. The assays proceed as outlined above for diagnosis. PCR method may provide more sensitive and accurate quantification.

### Assays for Therapeutic Compounds

In a preferred embodiment members of the three classes of proteins as described herein are used in drug screening assays. The metastatic breast cancer or metastatic lung cancer proteins, antibodies, nucleic acids, modified proteins and cells containing metastatic breast cancer or metastatic lung cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279:84-8 (1998); Heid, Genome Res 6:986-94, 1996).

In a preferred embodiment, the metastatic breast cancer or metastatic lung cancer proteins, antibodies, nucleic acids, modified proteins and cells containing the native or modified metastatic breast cancer or metastatic lung cancer proteins are used in screening assays. That is, the present invention provides novel methods for screening for compositions which modulate the metastatic breast cancer or metastatic lung cancer phenotype or an identified physiological function of a metastatic breast cancer or metastatic lung cancer protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokamik, supra.

Having identified the differentially expressed genes herein, a variety of assays may be applied. In a preferred embodiment, assays may be run on an individual gene or protein level. That is, having identified a particular gene with altered regulation in metastatic breast cancer or metastatic lung cancer, test compounds can be screened for the ability to modulate gene expression or for binding to the metastatic breast cancer or metastatic lung cancer protein. “Modulation” thus includes an increase or a decrease in gene expression. The preferred amount of modulation will depend on the original change of the gene expression in normal versus tissue undergoing metastatic breast cancer or metastatic lung cancer, with changes of at least 10%, preferably 50%, more preferably 100-300%, and in some embodiments 300-1000% or greater. Thus, if a gene exhibits a 4-fold increase in metastatic breast cancer or metastatic lung cancer tissue compared to normal tissue, a decrease of about four-fold is often desired; similarly, a 10-fold decrease in metastatic breast cancer or metastatic lung cancer tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the metastatic breast cancer or metastatic lung cancer protein and standard immunoassays. Proteomics and separation techniques may also allow quantification of expression.

In a preferred embodiment, gene or protein expression monitoring of a number of entities, i.e., an expression profile, is monitored simultaneously. Such profiles will typically involve a plurality of those entities described herein.

In this embodiment, the metastatic breast cancer or metastatic lung cancer nucleic acid probes are attached to biochips as outlined herein for the detection and quantification of metastatic breast cancer or metastatic lung cancer sequences in a particular cell. Alternatively, PCR may be used. Thus, a series, e.g., of microtiter plate, may be used with dispensed primers in desired wells. A PCR reaction can then be performed and analyzed for each well.

Expression monitoring can be performed to identify compounds that modify the expression of one or more metastatic breast cancer or metastatic lung cancer-associated sequences, e.g., a polynucleotide sequence set out in Tables 1-12. Generally, in a preferred embodiment, a test compound is added to the cells prior to analysis. Moreover, screens are also provided to identify agents that modulate metastatic breast cancer or metastatic lung cancer, modulate metastatic breast cancer or metastatic lung cancer proteins, bind to a metastatic breast cancer or metastatic lung cancer protein, or interfere with the binding of a metastatic breast cancer or metastatic lung cancer protein and an antibody, substrate, or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the metastatic breast cancer or metastatic lung cancer phenotype or the expression of a metastatic breast cancer or metastatic lung cancer sequence, e.g., a nucleic acid or protein sequence. In preferred embodiments, modulators alter expression profiles of nucleic acids or proteins provided herein. In one embodiment, the modulator suppresses a metastatic breast cancer or metastatic lung cancer phenotype, e.g., to a normal tissue fingerprint. In another embodiment, a modulator induces a metastatic breast cancer or metastatic lung cancer phenotype. Generally, a plurality of assay mixtures are run in parallel with different agent concentrations to obtain a differential response to the various concentrations. Typically, one of these concentrations serves as a negative control, i.e., at zero concentration or below the level of detection.

In one aspect, a modulator will neutralize the effect of a metastatic breast cancer or metastatic lung cancer protein. By “neutralize” is meant that activity of a protein and the consequent effect on the cell is inhibited or blocked.

In certain embodiments, combinatorial libraries of potential modulators will be screened for an ability to bind to a metastatic breast cancer or metastatic lung cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514, which is hereby incorporated by reference herein), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), nucleic acid libraries (see, e.g., Strategene, Corp.), peptide nucleic acid libraries (see, e.g., U.S. Pat. No. 5,539,083, which is hereby incorporated by reference herein), antibody libraries (see, e.g., Vaughn et al., Nature Biotechnology 14(3):309-314 (1996), and PCT/US96/10287), carbohydrate libraries (see, e.g., Liang et al., Science 274:1520-1522 (1996), and U.S. Pat. No. 5,593,853, which is hereby incorporated by reference herein), and small organic molecule libraries (see, e.g., benzodiazepines, Baum, C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like, each of which is hereby incorporated by reference herein).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J.; Asinex, Moscow, RU; Tripos, Inc., St. Louis, Mo.; ChemStar, Ltd, Moscow, RU; 3D Pharmaceuticals, Exton, Pa.; Martek Biosciences, Columbia, Md., etc.).

The assays to identify modulators are amenable to high throughput screening. Preferred assays thus detect modulation of metastatic breast cancer or metastatic lung cancer gene transcription, polypeptide expression, and polypeptide activity.

High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding. Each of the above-cited patents is hereby incorporated by reference herein.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that the nucleic acid or peptide consists of essentially random sequences of nucleotides and amino acids, respectively. Since these random peptides (or nucleic acids, discussed below) are often chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. In a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc.

Modulators of metastatic breast cancer or metastatic lung cancer can also be nucleic acids, as defined above.

As described above generally for proteins, nucleic acid modulating agents may be naturally occurring nucleic acids, random nucleic acids, or “biased” random nucleic acids. Digests of procaryotic or eucaryotic genomes may be used as is outlined above for proteins.

In a preferred embodiment, the candidate compounds are organic chemical moieties, a wide variety of which are available in the literature.

After a candidate agent has been added and the cells allowed to incubate for some period of time, the sample containing a target sequence is analyzed. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with Cy-3 or Cy-5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

Nucleic acid assays can be direct hybridization assays or can comprise “sandwich assays”, which include the use of multiple probes, as is generally outlined in U.S. Pat. No. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference herein. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697, which is hereby incorporated by reference herein. Thus, it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the metastatic breast cancer or metastatic lung cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product, or evaluate genetic polymorphisms.

Genes can be screened for those that are induced in response to a candidate agent. After identifying a modulator based upon its ability to suppress a metastatic breast cancer or metastatic lung cancer expression pattern leading to a normal expression pattern, or to modulate a single metastatic breast cancer or metastatic lung cancer gene expression profile so as to mimic the expression of the gene from normal tissue, a screen as described above can be performed to identify genes that are specifically modulated in response to the agent. Comparing expression profiles between normal tissue and agent treated metastatic breast cancer or metastatic lung cancer tissue reveals genes that are not expressed in normal tissue or metastatic breast cancer or metastatic lung cancer tissue, but are expressed in agent treated tissue. These agent-specific sequences can be identified and used by methods described herein for metastatic breast cancer or metastatic lung cancer genes or proteins. In particular these sequences and the proteins they encode find use in marking or identifying agent treated cells. In addition, antibodies can be raised against the agent induced proteins and used to target novel therapeutics to the treated metastatic breast cancer or metastatic lung cancer tissue sample.

Thus, in one embodiment, a test compound is administered to a population of metastatic breast cancer or metastatic lung cancer cells, that have an associated metastatic breast cancer or metastatic lung cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, e.g., metastatic breast cancer or metastatic lung cancer tissue may be screened for agents that modulate, e.g., induce or suppress the metastatic breast cancer or metastatic lung cancer phenotype. A change in at least one gene, preferably many, of the expression profile indicates that the agent has an effect on metastatic breast cancer or metastatic lung cancer activity. By defining such a signature for the metastatic breast cancer or metastatic lung cancer phenotype, screens for new drugs that alter the phenotype can be devised. With this approach, the drug target need not be known and need not be represented in the original expression screening platform, nor does the level of transcript for the target protein need to change.

Measure of metastatic breast cancer or metastatic lung cancer polypeptide activity, or of metastatic breast cancer or metastatic lung cancer or the metastatic breast cancer or metastatic lung cancer phenotype can be performed using a variety of assays. For example, the effects of the test compounds upon the function of the metastatic polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of metastatic breast cancer or metastatic lung cancer associated with tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian metastatic breast cancer or metastatic lung cancer polypeptide is typically used, e.g., mouse, preferably human.

To perform assays in intact animals where the breast or lung cancer has metastasized to the brain, it may be necessary to provide special treatments to facilitate crossing of the blood brain barrier by the metastatic cancer modulator or therapeutic. Any method known in the art can be used to achieve this objective.

Assays to identify compounds with modulating activity can be performed in vitro. For example, a metastatic breast cancer or metastatic lung cancer polypeptide is first contacted with a potential modulator and incubated for a suitable amount of time, e.g., from 0.5 to 48 hours. In one embodiment, the metastatic breast cancer or metastatic lung cancer polypeptide levels are determined in vitro by measuring the level of protein or mRNA. The level of protein is measured using immunoassays such as western blotting, ELISA and the like with an antibody that selectively binds to the metastatic breast cancer or metastatic lung cancer polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the metastatic breast cancer or metastatic lung cancer protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “metastatic breast cancer or metastatic lung cancer proteins.” The metastatic breast cancer or metastatic lung cancer protein may be a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the metastatic breast cancer or metastatic lung cancer proteins can be used in the assays.

Thus, in a preferred embodiment, the methods comprise combining a metastatic breast cancer or metastatic lung cancer protein and a candidate compound, and determining the binding of the compound to the metastatic breast cancer or metastatic lung cancer protein. Preferred embodiments utilize the human metastatic breast cancer or metastatic lung cancer protein, although other mammalian proteins may also be used, e.g., for the development of animal models of human disease. In some embodiments, as outlined herein, variant or derivative metastatic breast cancer or metastatic lung cancer proteins may be used.

Generally, in a preferred embodiment of the methods herein, the metastatic breast cancer or metastatic lung cancer protein or the candidate agent is non-diffusably bound to an insoluble support having isolated sample receiving areas (e.g., a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, TEFLON®, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the metastatic breast cancer or metastatic lung cancer protein is bound to the support, and a test compound is added to the assay. Alternatively, the candidate agent is bound to the support and the metastatic breast cancer or metastatic lung cancer protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the metastatic breast cancer or metastatic lung cancer protein may be done in a number of ways. In a preferred embodiment, the compound is labeled, and binding determined directly, e.g., by attaching all or a portion of the metastatic breast cancer or metastatic lung cancer protein to a solid support, adding a labeled candidate agent (e.g., a fluorescent label), washing off excess reagent, and determining whether the label is present on the solid support. Various blocking and washing steps may be utilized as appropriate.

In some embodiments, only one of the components is labeled, e.g., the proteins (or proteinaceous candidate compounds) can be labeled. Alternatively, more than one component can be labeled with different labels, e.g., 1251 for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e., a metastatic breast cancer or metastatic lung cancer protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the metastatic breast cancer or metastatic lung cancer protein and thus is capable of binding to, and potentially modulating, the activity of the metastatic breast cancer or metastatic lung cancer protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the metastatic breast cancer or metastatic lung cancer protein with a higher affinity. Thus, if the test compound is labeled, the presence of the label on the support, coupled with a lack of competitor binding, may indicate that the test compound is capable of binding to the metastatic breast cancer or metastatic lung cancer protein.

In a preferred embodiment, the methods comprise differential screening to identity agents that are capable of modulating the activity of the metastatic breast cancer or metastatic lung cancer proteins. In this embodiment, the methods comprise combining a metastatic breast cancer or metastatic lung cancer protein and a competitor in a first sample. A second sample comprises a test compound, a metastatic breast cancer or metastatic lung cancer protein, and a competitor. The binding of the competitor is determined for both samples, and a change, or difference in binding between the two samples indicates the presence of an agent capable of binding to the metastatic breast cancer or metastatic lung cancer protein and potentially modulating its activity. That is, if the binding of the competitor is different in the second sample relative to the first sample, the agent is capable of binding to the metastatic breast cancer or metastatic lung cancer protein.

Alternatively, differential screening is used to identify drug candidates that bind to the native metastatic breast cancer or metastatic lung cancer protein, but cannot bind to modified metastatic breast cancer or metastatic lung cancer proteins. The structure of the metastatic breast cancer or metastatic lung cancer protein may be modeled, and used in rational drug design to synthesize agents that interact with that site. Drug candidates that affect the activity of a metastatic breast cancer or metastatic lung cancer protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of a metastatic breast cancer or metastatic lung cancer protein. The methods comprise adding a test compound, as defined above, to a cell comprising metastatic breast cancer or metastatic lung cancer proteins. Preferred cell types include almost any cell. The cells contain a recombinant nucleic acid that encodes a metastatic breast cancer or metastatic lung cancer protein. In a preferred embodiment, a library of candidate agents are tested on a plurality of cells.

In one aspect, the assays are evaluated in the presence or absence or previous or subsequent exposure of physiological signals, e.g., hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate metastatic breast cancer or metastatic lung cancer agents are identified. Compounds with pharmacological activity are able to enhance or interfere with the activity of the metastatic breast cancer or metastatic lung cancer protein. Once identified, similar structures are evaluated to identify critical structural feature of the compound.

In one embodiment, a method of inhibiting metastatic breast cancer or metastatic lung cancer cell division is provided. The method comprises administration of a metastatic breast cancer or metastatic lung cancer inhibitor. In another embodiment, a method of inhibiting metastatic breast cancer or metastatic lung cancer is provided. The method comprises administration of a metastatic breast cancer or metastatic lung cancer inhibitor. In a further embodiment, methods of treating cells or individuals with metastatic breast cancer or metastatic lung cancer are provided. The method comprises administration of a metastatic breast cancer or metastatic lung cancer inhibitor.

A variety of cell growth, proliferation, and metastasis assays are known to those of skill in the art, as described below.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow. Soft agar growth or colony formation in suspension assays can be used to identify modulators of metastatic breast cancer or metastatic lung cancer sequences, which when expressed in host cells, inhibit abnormal cellular proliferation and transformation. A therapeutic compound would reduce or eliminate the host cells' ability to grow in stirred suspension culture or suspended in semi-solid media, such as semi-solid or soft.

Techniques for soft agar growth or colony formation in suspension assays are described in Freshney, Culture of Animal Cells a Manual of Basic Technique (3^{rd} ed., 1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with (³H)-thymidine at saturation density can be used to measure density limitation of growth. See Freshney (1994), supra. The transformed cells, when transfected with tumor suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a metastatic breast cancer or metastatic lung cancer-associated sequence and are grown for 24 hours at saturation density in non-limiting medium conditions. The percentage of cells labeling with (³H)-thymidine is determined autoradiographically. See, Freshney (1994), supra.

Growth factor or Serum Dependence

Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. Growth factor or serum dependence of transformed host cells can be compared with that of control.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. in Biological Responses in Cancer, pp. 178-184 (Mihich (ed.) 1985)). Similarly, Tumor angiogenesis factor (TAF) is released at a higher level in tumor cells than their normal counterparts. See, e.g., Folkman, Angiogenesis and Cancer, Sem Cancer Biol. (1992)).

Various techniques which measure the release of these factors are described in Freshney (1994), supra. Also, see, Unkless et al. , J. Biol. Chem. 249:4295-4305 (1974); Strickland & Beers, J. Biol. Chem. 251:5694-5702 (1976); Whur et al., Br. J. Cancer 42:305-312 (1980); Angiogenesis, tumor vascularization, and potential intereference with tumor growth. in Biological Responses in Cancer, pp. 178-184 (Mihich (ed.) 1985); Freshney Anticancer Res. 5:111-130 (1985).

Invasiveness into Matrigel

The degree of invasiveness into Matrigel or some other extracellular matrix constituent can be used as an assay to identify compounds that modulate metastatic breast cancer or metastatic lung cancer-associated sequences. Tumor cells exhibit a good correlation between malignancy and invasiveness of cells into Matrigel or some other extracellular matrix constituent. In this assay, tumorigenic cells are typically used as host cells. Expression of a tumor suppressor gene in these host cells would decrease invasiveness of the host cells.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

Tumor Growth in Vivo

Effects of metastatic breast cancer or metastatic lung cancer-associated sequences on cell growth can be tested in transgenic or immune-suppressed mice. Knock-out transgenic mice can be made, in which the metastatic breast cancer or metastatic lung cancer gene is disrupted or in which a metastatic breast cancer or metastatic lung cancer gene is inserted. Knock-out transgenic mice can be made by insertion of a marker gene or other heterologous gene into the endogenous metastatic breast cancer or metastatic lung cancer gene site in the mouse genome via homologous recombination. Such mice can also be made by substituting the endogenous metastatic breast cancer or metastatic lung cancer gene with a mutated version of the metastatic breast cancer or metastatic lung cancer gene, or by mutating the endogenous metastatic breast cancer or metastatic lung cancer gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J Cancer 41:52 (1980)) can be used as a host. Transplantable tumor cells (typically about 10⁶ cells) injected into isogenic hosts will produce invasive tumors in a high proportions of cases, while normal cells of similar origin will not. In hosts which developed invasive tumors, cells expressing a metastatic breast cancer or metastatic lung cancer-associated sequences are injected subcutaneously. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Additionally, human tumor cells expressing the genes of the invention may be injected into immune compromised animals. Growth of these tumors, or xenografts, is compared to growth of similar human tumor cell that do not express the genes of the invention. These animals may also be used to binding assays and efficacy studies for therapeutic compounds that modulate metastatic breast cancer or metastatic lung cancer, such as antibodies or small molecules.

### Polynucleotide Modulators of Metastatic Breast Cancer or Metastatic Lung Cancer

Antisense Polynucleotides

In certain embodiments, the activity of a metastatic breast cancer or metastatic lung cancer-associated protein is downregulated, or entirely inhibited, by the use of antisense polynucleotide, i.e., a nucleic acid complementary to, and which can preferably hybridize specifically to, a coding mRNA nucleic acid sequence, e.g., a metastatic breast cancer or metastatic lung cancer protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the metastatic breast cancer or metastatic lung cancer protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems (Norwalk, Conn.). The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for metastatic breast cancer or metastatic lung cancer molecules. A preferred antisense molecule is for a metastatic breast cancer or metastatic lung cancer sequence selected from those listed in Tables 1A-12C, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein & Cohen (Cancer Res. 48:2659 (1988 and van der Krol et al. (BioTechniques 6:958 (1988)).

Ribozymes

In addition to antisense polynucleotides, ribozymes can be used to target and inhibit transcription of metastatic breast cancer or metastatic lung cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different ribozymes).

The general features of hairpin ribozymes are described, e.g., in Hampel et al., Nucl. Acids Res. 18:299-304 (1990); European Patent Publication No. 0 360 257; U.S. Pat. No. 5,254,678, which is hereby incorporated by reference herein. Methods of preparing are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

Polynucleotide modulators of metastatic breast cancer or metastatic lung cancer may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of metastatic breast cancer or metastatic lung cancer may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating metastatic breast cancer or metastatic lung cancer in cells or organisms are provided. In one embodiment, the methods comprise administering to a cell an anti-metastatic breast cancer or metastatic lung cancer antibody that reduces or eliminates the biological activity of an endogenous metastatic breast cancer or metastatic lung cancer protein. Alternatively, the methods comprise administering to a cell or organism a recombinant nucleic acid encoding a metastatic breast cancer or metastatic lung cancer protein. This may be accomplished in any number of ways. In a preferred embodiment, e.g., when the metastatic breast cancer or metastatic lung cancer sequence is down-regulated in metastatic breast cancer or metastatic lung cancer, such state may be reversed by increasing the amount of metastatic breast cancer or metastatic lung cancer gene product in the cell. This can be accomplished, e.g., by overexpressing the endogenous metastatic breast cancer or metastatic lung cancer gene or administering a gene encoding the metastatic breast cancer or metastatic lung cancer sequence, using known gene-therapy techniques. In a preferred embodiment, the gene therapy techniques include the incorporation of the exogenous gene using enhanced homologous recombination (EHR), e.g., as described in PCT/US93/03868, hereby incorporated by reference in its entirety. Alternatively, e.g., when the metastatic breast cancer or metastatic lung cancer sequence is up-regulated in metastatic breast cancer or metastatic lung cancer, the activity of the endogenous metastatic breast cancer or metastatic lung cancer gene is decreased, e.g., by the administration of a metastatic breast cancer or metastatic lung cancer antisense nucleic acid.

In one embodiment, the metastatic breast cancer or metastatic lung cancer proteins of the present invention may be used to generate polyclonal and monoclonal antibodies to metastatic breast cancer or metastatic lung cancer proteins. Similarly, the metastatic breast cancer or metastatic lung cancer proteins can be coupled, using standard technology, to affinity chromatography columns. These columns may then be used to purify metastatic breast cancer or metastatic lung cancer antibodies useful for production, diagnostic, or therapeutic purposes. In a preferred embodiment, the antibodies are generated to epitopes unique to a metastatic breast cancer or metastatic lung cancer protein; that is, the antibodies show little or no cross-reactivity to other proteins. The metastatic breast cancer or metastatic lung cancer antibodies may be coupled to standard affinity chromatography columns and used to purify metastatic breast cancer or metastatic lung cancer proteins. The antibodies may also be used as blocking polypeptides, as outlined above, since they will specifically bind to the metastatic breast cancer or metastatic lung cancer protein.

### Methods of Identifying Variant Metastatic Breast Cancer or Metastatic Lung Cancer-associated Sequences

Without being bound by theory, expression of various metastatic breast cancer or metastatic lung cancer sequences is correlated with metastatic breast cancer or metastatic lung cancer. Accordingly, disorders based on mutant or variant metastatic breast cancer or metastatic lung cancer genes may be determined. In one embodiment, the invention provides methods for identifying cells containing variant metastatic breast cancer or metastatic lung cancer genes, e.g., determining all or part of the sequence of at least one endogenous metastatic breast cancer or metastatic lung cancer genes in a cell. This may be accomplished using any number of sequencing techniques. In a preferred embodiment, the invention provides methods of identifying the metastatic breast cancer or metastatic lung cancer genotype of an individual, e.g., determining all or part of the sequence of at least one metastatic breast cancer or metastatic lung cancer gene of the individual. This is generally done in at least one tissue of the individual, and may include the evaluation of a number of tissues or different samples of the same tissue. The method may include comparing the sequence of the sequenced metastatic breast cancer or metastatic lung cancer gene to a known metastatic breast cancer or metastatic lung cancer gene, i.e., a wild-type gene.

The sequence of all or part of the metastatic breast cancer or metastatic lung cancer gene can then be compared to the sequence of a known metastatic breast cancer or metastatic lung cancer gene to determine if any differences exist. This can be done using any number of known homology programs, such as Bestfit, etc. In a preferred embodiment, the presence of a difference in the sequence between the metastatic breast cancer or metastatic lung cancer gene of the patient and the known metastatic breast cancer or metastatic lung cancer gene correlates with a disease state or a propensity for a disease state, as outlined herein.

In a preferred embodiment, the metastatic breast cancer or metastatic lung cancer genes are used as probes to determine the number of copies of the metastatic breast cancer or metastatic lung cancer gene in the genome.

In another preferred embodiment, the metastatic breast cancer or metastatic lung cancer genes are used as probes to determine the chromosomal localization of the metastatic breast cancer or metastatic lung cancer genes. Information such as chromosomal localization finds use in providing a diagnosis or prognosis in particular when chromosomal abnormalities such as translocations, and the like are identified in the metastatic breast cancer or metastatic lung cancer gene locus.

### Administration of Pharmaceutical and Vaccine Compositions

In one embodiment, a therapeutically effective dose of a metastatic breast cancer or metastatic lung cancer protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of pharmaceutical Compounding (1999); and Pickar, Dosage Calculations (1999)). As is known in the art, adjustments for metastatic breast cancer or metastatic lung cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the metastatic breast cancer or metastatic lung cancer proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the metastatic breast cancer or metastatic lung cancer proteins and modulators may be directly applied as a solution or spray.

The pharmaceutical compositions of the present invention comprise a metastatic breast cancer or metastatic lung cancer protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, ftimaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that metastatic breast cancer or metastatic lung cancer protein modulators (e.g., antibodies, antisense constructs, ribozymes, small organic molecules, etc.) when administered orally, should be protected from digestion. It is also recognized that, after delivery to other sites in the body (e.g., circulatory system, lymphatic system, or the tumor site) the metastatic breast cancer or metastatic lung cancer modulators of the invention may need to be protected from excretion, hydrolisis, proteolytic digestion or modification, or detoxification by the liver. In all these cases, protection is typically accomplished either by complexing the molecule(s) with a composition to render it resistant to acidic and enzymatic. hydrolysis, or by packaging the molecule(s) in an appropriately resistant carrier, such as a liposome or a protection barrier or by modifying the molecular size, weight, and/or charge of the modulator. Means of protecting agents from digestion degradation, and excretion are well known in the art.

The compositions for administration will commonly comprise a metastatic breast cancer or metastatic lung cancer protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacologial Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gillman, The Pharmacologial Basis of Therapeutics, supra.

The compositions containing modulators of metastatic breast cancer or metastatic lung cancer proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present metastatic breast cancer or metastatic lung cancer protein-modulating compounds can be administered alone or in combination with additional metastatic breast cancer or metastatic lung cancer modulating compounds or with other therapeutic agent, e.g., other anti-cancer agents or treatments.

In numerous embodiments, one or more nucleic acids, e.g., polynucleotides comprising nucleic acid sequences set forth in Tables 1A-12C, such as antisense polynucleotides or ribozymes, will be introduced into cells, in vitro or in vivo. The present invention provides methods, reagents, vectors, and cells useful for expression of metastatic breast cancer or metastatic lung cancer-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger & Kimmel, Guide to Molecular Cloning Techniques, Methods in Enzymology volume 152 (Berger), Ausubel et al., eds., Current Protocols (supplemented through 1999), and Sambrook et al., Molecular Cloning—A Laboratory Manual (2nd ed., Vol. 1-3, 1989.

In a preferred embodiment, metastatic breast cancer or metastatic lung cancer proteins and modulators are administered as therapeutic agents, and can be formulated as outlined above. Similarly, metastatic breast cancer or metastatic lung cancer genes (including both the full-length sequence, partial sequences, or regulatory sequences of the metastatic breast cancer or metastatic lung cancer coding regions) can be administered in a gene therapy application. These metastatic breast cancer or metastatic lung cancer genes can include antisense applications, either as gene therapy (i.e., for incorporation into the genome) or as antisense compositions, as will be appreciated by those in the art.

Metastatic breast cancer or metastatic lung cancer polypeptides and polynucleotides can also be administered as vaccine compositions to stimulate HTL, CTL and antibody responses. Such vaccine compositions can include, e.g., lipidated peptides (see, e.g.,Vitiello, et al., J. Clin. Invest. 95:341 (1995)), peptide compositions encapsulated in poly(DL-lactide-co-glycolide) (“PLG”) microspheres (see, e.g., Eldridge, et al., Molec. Immunol. 28:287-294, (1991); Alonso et al., Vaccine 12:299-306 (1994); Jones et al., Vaccine 13:675-681 (1995)), peptide compositions contained in immune stimulating complexes (ISCOMS) (see, e.g., Takahashi et al., Nature 344:873-875 (1990); Hu et al., Clin Exp Immunol. 113:235-243 (1998)), multiple antigen peptide systems (MAPs) (see, e.g., Tam, Proc. Natl. Acad. Sci. U.S.A. 85:5409-5413 (1988); Tam, J. Immunol. Methods 196:17-32 (1996)), peptides formulated as multivalent peptides; peptides for use in ballistic delivery systems, typically crystallized peptides, viral delivery vectors (Perkus, et al., In: Concepts in vaccine development (Kaufmann, ed., p. 379, 1996); Chakrabarti, et al., Nature 320:535 (1986); Hu et al., Nature 320:537 (1986); Kieny, et al., AIDS Bio/Technology 4:790 (1986); Top et al., J. Infect. Dis. 124:148 (1971); Chanda et al., Virology 175:535 (1990)), particles of viral or synthetic origin (see, e.g., Kofler et al., J. Immunol. Methods. 192:25 (1996); Eldridge et al., Sem. Hematol. 30:16 (1993); Falo et al., Nature Med. 7:649 (1995)), adjuvants (Warren et al., Annu. Rev. Immunol. 4:369 (1986); Gupta et al., Vaccine 11:293 (1993)), liposomes (Reddy et al., J. Immunol. 148:1585 (1992); Rock, Immunol. Today 17:131 (1996)), or, naked or particle absorbed cDNA (Ulmer, et al., Science 259:1745 (1993); Robinson et al., Vaccine 11:957 (1993); Shiver et al., In: Concepts in vaccine development (Kaufinann, ed., p. 423, 1996); Cease & Berzofsky, Annu. Rev. Immunol. 12:923 (1994) and Eldridge et al., Sem. Hematol. 30:16 (1993)). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720, each of which is hereby incorporated by reference herein.; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687, which is hereby incorporated by reference herein.).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode metastatic breast cancer or metastatic lung cancer polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848, which is hereby incorporated by reference herein. Another vector is BCG (Bacille Calmette Guerin). BCG vectors are described in Stover et al., Nature 351:456-460 (1991). A wide variety of other vectors useful for therapeutic administration or immunization e.g., adeno and adeno-associated virus vectors, retroviral vectors, Salmonella typhi vectors, detoxified anthrax toxin vectors, and the like, will be apparent to those skilled in the art from the description herein (see, e.g., Shata et al., Mol Med Today 6:66-71 (2000); Shedlock et al., J Leukoc Biol 68:793-806 (2000); Hipp et al., In Vivo 14:571-85 (2000)).

Methods for the use of genes as DNA vaccines are well known, and include placing a metastatic breast cancer or metastatic lung cancer gene or portion of a metastatic breast cancer or metastatic lung cancer gene under the control of a regulatable promoter or a tissue-specific promoter for expression in a metastatic breast cancer or metastatic lung cancer patient. The metastatic breast cancer or metastatic lung cancer gene used for DNA vaccines can encode full-length metastatic breast cancer or metastatic lung cancer proteins, but more preferably encodes portions of the metastatic breast cancer or metastatic lung cancer proteins including peptides derived from the metastatic breast cancer or metastatic lung cancer protein. In one embodiment, a patient is immunized with a DNA vaccine comprising a plurality of nucleotide sequences derived from a metastatic breast cancer or metastatic lung cancer gene. For example, metastatic breast cancer or metastatic lung cancer-associated genes or sequence encoding subfragments of a metastatic breast cancer or metastatic lung cancer protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the metastatic breast cancer or metastatic lung cancer polypeptide encoded by the DNA vaccine. Additional or alternative adjuvants are available.

In another preferred embodiment metastatic breast cancer or metastatic lung cancer genes find use in generating animal models of metastatic breast cancer or metastatic lung cancer. When the metastatic breast cancer or metastatic lung cancer gene identified is repressed or diminished in metastatic tissue, gene therapy technology, e.g., wherein antisense RNA directed to the metastatic breast cancer or metastatic lung cancer gene will also diminish or repress expression of the gene. Animal models of metastatic breast cancer or metastatic lung cancer find use in screening for modulators of a metastatic breast cancer or metastatic lung cancer-associated sequence or modulators of metastatic breast cancer or metastatic lung cancer. Similarly, transgenic animal technology including gene knockout technology, e.g., as a result of homologous recombination with an appropriate gene targeting vector, will result in the absence or increased expression of the metastatic breast cancer or metastatic lung cancer protein. When desired, tissue-specific expression or knockout of the metastatic breast cancer or metastatic lung cancer protein may be necessary.

It is also possible that the metastatic breast cancer or metastatic lung cancer protein is overexpressed in metastatic breast cancer or metastatic lung cancer. As such, transgenic animals can be generated that overexpress the metastatic breast cancer or metastatic lung cancer protein. Depending on the desired expression level, promoters of various strengths can be employed to express the transgene. Also, the number of copies of the integrated transgene can be determined and compared for a determination of the expression level of the transgene. Animals generated by such methods find use as animal models of metastatic breast cancer or metastatic lung cancer and are additionally useful in screening for modulators to treat metastatic breast cancer or metastatic lung cancer.

### Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, metastatic breast cancer or metastatic lung cancer-specific nucleic acids or antibodies, hybridization probes and/or primers, antisense polynucleotides, ribozymes, dominant negative metastatic breast cancer or metastatic lung cancer polypeptides or polynucleotides, small molecules inhibitors of metastatic breast cancer or metastatic lung cancer-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of metastatic breast cancer or metastatic lung cancer-associated sequences. Such kits can be prepared from readily available materials and reagents. For example, such kits can comprise one or more of the following materials: a metastatic breast cancer or metastatic lung cancer- associated polypeptide or polynucleotide, reaction tubes, and instructions for testing metastatic breast cancer or metastatic lung cancer-associated activity. Optionally, the kit contains biologically active metastatic breast cancer or metastatic lung cancer protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.
Table 1A shows about 461 genes upregulated in breast metastases to the brain relalve to normal breast tissues. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 2A shows about 445 genes upregulated in breast metastases to the brain relatve to normal body tissues. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 3A shows about 216 genes upregulated in breast metastases to the brain relative to primary breast tumors. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 4A shows about 350 genes downregulated in breast metastases to the brain relaive to primary breast tumors. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 5A shows about 489 genes downregulated in breast metastases to the brain relative to normal breast tissue. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 6A shows about 1251 genes upregulated in lung metastases to the brain relaive to normal lung tissues. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 7A shows about 381 genes upregulated in lung metastases to the brain relaive to normal body tissues. These genes were selected from 59680 probesets on the Eos/Affymetrix Hu03 Genechip array. Gene expression data for each probeset obtained from this analysis was expressed as average intensity (Al), a normalized value refleting the relative level of mRNA expression.
Table 8A shows about 330 genes upregulated in lung metastases to the brain relaive to pomary lung tumors. These genes were selected from 59680 probesets on the Eos/Affymetrix Hu03 Genechip array. Gene expression data for each probeset obtained from this analysis was expressed as average intensity (Al), a normalized value refleting the relative level of mRNA expression.
Table 9A shows about 252 genes downregulated in lung metastases to the brain relaive to primary lung tumors. These genes were selected from 59680 probesets on the Eos/Affymetrix Hu03 Genechip array. Gene expression data for each probeset obtained from this analysis was expressed as average intensity (Al), a normalized value refleting the relative level of mRNA expression.
Table 10A shows about 289 genes downregulated in lung metastases to the brain relative to normal lung tissue. These genes were selected from 59680 probesets on the Eos/Affymetrix Hu03 Genechip array. Gene expression data for each probeset obtained from this analysis was expressed as average intensity (Al), a norrmalized value refleting the relative level of mRNA expression.
Table 11A shows about 1198 genes upregulated in breast and lung metastases to the brain relative to normal body tissues. These genes were selected from 59680 probesets on the Ecos/Affymetix Hu03 Genechip array. Gene expression data for each probeset obtained from this analysis was expressed as average intensity (Al), a normalized value refleting the relative level of mRNA expression.
Table 12A shows about 2867 genes upregulated in breast and lung metastases to the brain relative to normal breast and lung tissues. These genes were selected from 59680 probesets on the Eos/Affymetrix Hu03 Genechip array. Gene expression data for each probeset obtained from this analysis was expressed as average intensity (Al), a normalized value reflecting the relative level of mRNA expression.
Table 1B-12C shows the accession numbers for those pkeys in Tables 1A-12A lacking unigenelD's. For each probeset we have listed the gene cluter number from which the oligonucleotides were designed. Gene clusters were compiled using sequences derived from Genbank ESTs and mRNAs. These sequences were clustered based on sequence similary using Clustering and Alignment Tools (DoubleTwist, Oakland Calif.). The Genbank accession numbers for sequences comprising each cluster are listed in the Accessoin column.

Tables 1C-12C shows genomic positioning for those pkeys lacking unigene ID's and accession numbers in tables 1A-12A. For each predicted exon, we have listed the genomic sequence source used for prediction. Nucleotide locations of each predicted exon are also listed.

TABLE 1A
About 461 genes upregulated in breast metastases to the brain relative to normal breast tissues

| Pkey | ExAccn | UniGeneID | UniGene Title | R1 | SEQ ID NO(s): |
|---|---|---|---|---|---|
| 419741 | NM_007019 | Hs.93002 | ubiquitin carrier protein E2-C | 23.16 | 1355 5056 |
| 420542 | NM_000505 | Hs.1321 | coagulation factor XII (Hageman factor) | 18.65 | 1428 5080 |
| 407014 | U38268 | | gb: Human cytochrome b pseudogene, partia | 17.68 | 118 |
| 405452 | | | Target Exon | 16.40 | |
| 417900 | BE250127 | Hs.82906 | CDC20 (cell division cycle 20, S. cerevi | 14.23 | 1136 |
| 419078 | M93119 | Hs.89584 | insulinoma-associated 1 | 14.05 | 1272 5036 |
| 421693 | X71490 | Hs.106876 | ATPase, H transporting, lysosomal (vacuo | 13.45 | 1555 5120 |
| 422765 | AW409701 | Hs.1578 | baculoviral IAP repeat-containing 5 (sur | 12.17 | 1680 |
| 408908 | BE296227 | Hs.250822 | serine/threonine kinase 15 | 11.78 | 317 |
| 410407 | X66839 | Hs.63287 | carbonic anhydrase IX | 11.66 | 474 4846 |
| 418836 | AI655499 | Hs.161712 | BMP-R1B | 11.53 | 1247 |
| 419875 | AA853410 | Hs.93557 | proenkephalin | 10.59 | 1365 |
| 412513 | AA322599 | Hs.5163 | ESTs, Weakly similar to AF151840 1 CGI-8 | 10.50 | 640 |
| 400205 | | | NM_006265*: Homo sapiens RAD21 (S. pombe) | 10.40 | 1 4680 |
| 443426 | AF098158 | Hs.9329 | chromosome 20 open reading frame 1 | 10.28 | 3523 5627 |
| 426842 | NM_004210 | Hs.172700 | neuralized (Drosophila)-like | 10.07 | 2106 5324 |
| 414358 | AA476456 | Hs.142614 | ESTs | 10.05 | 815 |
| 427324 | AA159587 | Hs.285932 | hypothetical protein FLJ23322 | 9.90 | 2142 |
| 431070 | AW408164 | Hs.249184 | transcription factor 19 (SC1) | 9.68 | 2529 |
| 425397 | J04088 | Hs.156346 | topoisomerase (DNA) II alpha (170 kD) | 9.53 | 1959 5261 |
| 430375 | AW371048 | Hs.93758 | H4 histone family, member H | 9.36 | 2477 |
| 417308 | H60720 | Hs.81892 | KIAA0101 gene product | 9.35 | 1079 |
| 429503 | AA394183 | Hs.204166 | ESTs | 9.35 | 2381 |
| 432178 | BE265369 | Hs.272814 | hypothetical protein DKFZp434E1723 | 9.33 | 2639 |
| 444371 | BE540274 | Hs.239 | forkhead box M1 | 9.14 | 3592 |
| 407777 | AA161071 | Hs.71465 | squalene epoxidase | 9.09 | 194 |
| 414907 | X90725 | Hs.77597 | polo (Drosophia)-like kinase | 8.73 | 4933 891 |
| 430294 | AI538226 | Hs.32976 | guanine nucleotide binding protein 4 | 8.52 | 2463 |
| 400914 | | | ENSP00000228091*: Calcium-binding protein | 8.50 | |
| 423198 | M81933 | Hs.1634 | cell division cycle 25A | 8.49 | 1727 5174 |
| 434916 | AF161383 | Hs.284207 | Homo sapiens, Similar to RIKEN cDNA 1110 | 8.30 | 2879 5558 |
| 447342 | AI199268 | Hs.19322 | Homo sapiens, Similar to RIKEN cDNA 2010 | 8.12 | 3845 |
| 400292 | AA250737 | Hs.72472 | BMP-R1B (bone morphogenetic protein rec | 8.07 | 6 |
| 419390 | AI701162 | Hs.90207 | hypothetical protein MGC11138 | 7.98 | 1309 |
| 403532 | | | NM_024638: Homo sapiens hypothetical prot | 7.97 | 46 4715 |
| 402542 | | | Target Exon | 7.97 | |
| 422799 | AI933199 | Hs.120911 | neurexophilin 4 | 7.95 | 1682 |
| 421506 | BE302796 | Hs.105097 | thymidine kinase 1, soluble | 7.79 | 1528 |
| 436877 | AA931484 | Hs.121255 | ESTs, Weakly similar to T21069 hypotheti | 7.73 | 3017 |
| 414432 | BE378174 | Hs.26506 | Homo sapiens clone CDABP0005 mRNA sequen | 7.56 | 829 |
| 406690 | M29540 | Hs.220529 | carcinoembryonic antigen-related cell ad | 7.56 | 4748 86 |
| 428865 | BE544095 | Hs.164960 | BarH-like homeobox 1 | 7.54 | 2314 |
| 437929 | T09353 | Hs.106642 | ESTs, Weakly similar to T09052 hypotheti | 7.50 | 3108 |
| 411006 | AW813193 | Hs.17767 | KIAA1554 protein | 7.43 | 526 |
| 453439 | AI572438 | Hs.32976 | guanine nucleotide binding protein 4 | 7.40 | 4406 |
| 451930 | BE259124 | Hs.27262 | Homo sapiens clone 25110 mRNA sequence | 7.40 | 4257 |
| 448409 | AW069807 | Hs.134726 | ESTs, Moderately similar to PC4259 ferri | 7.37 | 3966 |
| 412140 | AA219691 | Hs.73625 | RAB6 interacting, kinesin-like (rabkines | 7.36 | 613 |
| 433272 | AB043585 | Hs.100890 | candidate mediator of the p53-dependent | 7.34 | 2752 5534 |
| 457465 | AW301344 | Hs.122908 | DNA replication factor | 7.33 | 4592 |
| 422961 | Y13620 | Hs.122607 | B-cell CLL/lymphoma 9 | 7.29 | 1700 5163 |
| 436876 | AI124756 | Hs.5337 | isocitrate dehydrogenase 2 (NADP), mitoc | 7.26 | 3016 |
| 401451 | | | NM_004496*: Homo sapiens hepatocyte nucle | 7.24 | 27 4697 |
| 457211 | AW972565 | Hs.32399 | ESTs, Weakly similar to S51797 vasodilat | 7.16 | 4583 |
| 429353 | AL117406 | Hs.335891 | ATP-binding cassette transporter MRP8 | 7.14 | 2359 |
| 451346 | NM_006338 | Hs.26312 | glioma amplified on chromosome 1 protein | 7.12 | 4213 5743 |
| 428648 | AF052728 | Hs.188021 | potassium voltage-gated channel, subfami | 7.10 | 2279 5369 |
| 453028 | AB006532 | Hs.31442 | RecQ protein-like 4 | 7.07 | 4368 5768 |
| 423551 | AA327598 | Hs.89633 | ESTs | 7.07 | 1757 |
| 453968 | AA847843 | Hs.62711 | High mobility group (nonhistone chromoso | 7.00 | 4456 |
| 421487 | AF027406 | Hs.104865 | serine/threonine kinase 23 | 6.98 | 1526 5109 |
| 425371 | D49441 | Hs.155981 | mesothelin | 6.96 | 1957 5259 |
| 443347 | AI052543 | Hs.133244 | melanoma-derived leucine zipper, extra-n | 6.95 | 3519 |
| 421458 | NM_003654 | Hs.104576 | carbohydrate (keratan sulfate Gal-6) sul | 6.84 | 1521 5107 |
| 402265 | | | Target Exon | 6.82 | |
| 432180 | Y18418 | Hs.272822 | RuvB (E coli homolog)-like 1 | 6.70 | 2640 5502 |
| 403291 | | | Target Exon | 6.67 | |
| 412856 | BE386745 | Hs.74631 | basigin (OK blood group) | 6.66 | 678 |
| 453392 | U23752 | Hs.32964 | SRY (sex determining region Y)-box 11 | 6.65 | 4403 5776 |
| 401076 | | | C11000393: gi\|7305361\|ref\|NP_038652.1\|ot | 6.65 | |
| 428484 | AF104032 | Hs.184601 | solute carrier family 7 (cationic amino | 6.64 | 2265 5364 |
| 422296 | AA360231 | Hs.114416 | Homo sapiens , Similar to transducin (bet | 6.64 | 1629 |
| 427914 | AA417350 | Hs.20575 | ESTs | 6.59 | 2204 |
| 436291 | BE568452 | Hs.344037 | protein regulator of cytokinesis 1 | 6.57 | 2975 |
| 414064 | BE245289 | Hs.16165 | expressed in activated T/LAK lymphocytes | 6.56 | 784 |
| 402907 | | | NM_024777*: Homo sapiens hypothetical pro | 6.55 | |
| 458814 | AI498957 | Hs.170861 | ESTs, Weakly similar to Z195_HUMAN ZINC | 6.55 | 4638 |
| 426686 | AI362802 | Hs.171814 | parathymosin | 6.52 | 2087 |
| 402078 | | | Target Exon | 6.47 | |
| 407168 | R45175 | Hs.117183 | ESTs | 6.46 | 131 |
| 426553 | AA381293 | Hs.23598 | ESTs | 6.45 | 2077 |
| 403988 | | | C5001831: gi\|11056014\|ref\|NP_067651.1\|ac | 6.40 | |
| 452969 | W92792 | Hs.77575 | hypothetical protein MGC3136 | 6.37 | 4361 |
| 402961 | | | Homo sapiens mRNA; cDNA DKFZp761E0611 (f | 6.33 | |
| 422938 | NM_001809 | Hs.1594 | centromere protein A (17 kD) | 6.32 | 1694 5162 |
| 402217 | | | C19001662*: gi\|6753872\|ref\|NP_034345.1\|i | 6.32 | |
| 432221 | M21191 | Hs.273415 | aldolase A, fructose-bisphosphate | 6.30 | 2644 |
| 443723 | AI144442 | Hs.157144 | syntaxin 6 | 6.27 | 3545 |
| 419081 | AI798863 | Hs.87191 | ESTs | 6.27 | 1273 |
| 402649 | | | Target Exon | 6.20 | |
| 400183 | | | Eos Control | 6.20 | |
| 415262 | H95572 | Hs.206521 | YME1 (S. cerevisiae)-like 1 | 6.09 | 919 |
| 421582 | AI910275 | | trefoil factor 1 (breast cancer, extroge | 6.08 | 1541 |
| 405046 | | | C3000978: gi\|9280045\|dbj\|BAB01579.1\| (AB0 | 6.08 | |
| 409015 | BE389387 | Hs.49767 | NM_004553: Homo sapiens NADH dehydrogenas | 6.06 | 323 |
| 424047 | AI868401 | Hs.138248 | hypothetical protein YH95C04 | 6.05 | 1795 |
| 420005 | AW271106 | Hs.133294 | ESTs | 6.03 | 1372 |
| 403026 | | | Target Exon | 6.03 | |
| 430532 | D61216 | Hs.18672 | ESTs | 6.02 | 2494 |
| 430167 | Y08976 | Hs.234759 | FEV protein | 6.02 | 2448 5437 |
| 421242 | AW161386 | Hs.13561 | hypothetical protein MGC4692 | 6.01 | 1494 |
| 427239 | BE270447 | | ubiquitin carrier protein | 5.99 | 2134 |
| 447946 | AI566164 | Hs.277445 | ESTs | 5.97 | 3923 |
| 449722 | BE280074 | Hs.23960 | cyclin B1 | 5.95 | 4079 |
| 423226 | AA323414 | Hs.146109 | ESTs, Weakly similar to T28937 hypotheti | 5.94 | 1729 |
| 439963 | AW247529 | Hs.6793 | platelet-activating factor acetylhydrola | 5.92 | 3250 |
| 445470 | AI239871 | Hs.154758 | ESTs | 5.91 | 3677 |
| 403804 | | | Target Exon | 5.90 | |
| 428450 | NM_014791 | Hs.184339 | KIAA0175 gene product | 5.89 | 2259 5359 |
| 406947 | L10403 | Hs.3134 | DNA-binding protein amplifying expressio | 5.85 | 113 4759 |
| 418951 | F07809 | Hs.89506 | paired box gene 6 (aniridia, keratitis) | 5.85 | 1262 |
| 406137 | | | NM_000179*: Homo sapiens mutS (E. coli) h | 5.79 | 4742 76 |
| 414416 | AW409985 | Hs.76084 | hypothetical protein MGC2721 | 5.78 | 824 |
| 415539 | AI733881 | Hs.72472 | BMP-R1B (bone morphogenetic protein rec | 5.77 | 935 |
| 441761 | AI222880 | | gb: qp40c06.x1 NCI_CGAP_Co8 Homo sapiens | 5.77 | 3371 |
| 449644 | AW960707 | Hs.148324 | ESTs | 5.77 | 4072 |
| 429901 | AK000502 | Hs.56237 | hypothetical protein FLJ20495 | 5.75 | 2424 5429 |
| 418526 | BE019020 | Hs.85838 | solute carrier family 16 (monocarboxylic | 5.75 | 1211 |
| 421625 | AA405386 | Hs.178004 | ESTs | 5.74 | 1545 |
| 405146 | | | C8001690*: gi\|6754446\|ref\|NP_034760.1\|ki | 5.66 | |
| 424441 | X14850 | Hs.147097 | H2A histone family, member X | 5.65 | 1846 5212 |
| 443792 | AI763073 | Hs.204873 | ESTs | 5.63 | 3553 |
| 457341 | BE181716 | | gb: QV1-HT0639-150500-198-e03 HT0639 Homo | 5.62 | 4588 |
| 403384 | | | C4000351*: gi\|8394456\|ref\|NP_059138.1\|to | 5.62 | |
| 408157 | AA047685 | Hs.62946 | ESTs | 5.60 | 239 |
| 405968 | | | Target Exon | 5.60 | |
| 407701 | AW375009 | Hs.164407 | ESTs | 5.58 | 183 |
| 432917 | NM_014125 | Hs.241517 | PRO0327 protein | 5.57 | 2712 5518 |
| 422168 | AA586894 | Hs.112408 | S100 calcium-binding protein A7 (psorias | 5.56 | 1612 |
| 418322 | AA284166 | Hs.84113 | cyclin-dependent kinase inhibitor 3 (CDK | 5.55 | 1184 |
| 433157 | AW769671 | | ESTs, Moderately similar to CBX4_HUMAN C | 5.55 | 2741 |
| 400222 | | | NM_002082*: Homo sapiens G protein-couple | 5.54 | 3 4682 |
| 419444 | NM_002496 | Hs.90443 | Target CAT | 5.54 | 1314 5048 |
| 421937 | AI878857 | Hs.109706 | hematological and neurological expressed | 5.54 | 1582 |
| 441153 | BE562826 | | gb: 601336534F1 NIH_MGC_44 Homo sapiens c | 5.53 | 3336 |
| 429671 | BE379335 | Hs.211594 | proteasome (prosome, macropain) 26S subu | 5.53 | 2405 |
| 407242 | M18728 | | gb: Human nonspecific crossreacting antig | 5.53 | 142 4766 |
| 429469 | M64590 | Hs.27 | glycine dehydrogenase (decarboxylating; | 5.53 | 2374 5408 |
| 400340 | AJ223798 | | homeo box 11-like 2 | 5.53 | 13 4686 |
| 405467 | | | Target Exon | 5.50 | |
| 426081 | M69238 | Hs.166172 | aryl hydrocarbon receptor nuclear transI | 5.49 | 2023 5288 |
| 404321 | NA | | C7001741*: gi\|2499629\|sp\|Q63932\|MPK2_MOUS | 5.49 | |
| 439573 | AW137640 | Hs.231444 | Homo sapiens, Similar to hypothetical pr | 5.48 | 3218 |
| 437433 | R74016 | Hs.121581 | ESTs | 5.47 | 3064 |
| 440475 | AI807671 | Hs.24040 | potassium channel, subfamily K, member 3 | 5.46 | 3291 |
| 438552 | AJ245820 | Hs.6314 | type I transmembrane receptor (seizure-r | 5.42 | 3148 5596 |
| 403882 | | | Target Exon | 5.42 | |
| 411734 | AW374954 | Hs.71779 | Homo sapiens DNA from chromosome 19, cos | 5.40 | 572 |
| 412377 | AW947540 | | gb: RC0-MT0002-140300-011-g10 MT0002 Homo | 5.38 | 627 |
| 432562 | BE531048 | Hs.278422 | DKFZP586G1122 protein | 5.38 | 2680 |
| 411358 | R47479 | Hs.94761 | KIAA1691 protein | 5.38 | 548 |
| 416361 | AW204907 | Hs.6872 | ESTs, Weakly similar to CA13_HUMAN COLLA | 5.37 | 989 |
| 418514 | AW068487 | Hs.25413 | TOLLIP protein | 5.37 | 1209 |
| 443537 | D13305 | Hs.203 | cholecystokinin B receptor | 5.35 | 3528 5629 |
| 445329 | AW206378 | Hs.144809 | ESTs | 5.35 | 3670 |
| 452700 | AI859390 | Hs.288940 | five-span transmembrane protein M83 | 5.34 | 4336 |
| 438364 | AK000860 | Hs.6191 | hypothetical protein DKFZp7621166 | 5.33 | 3135 |
| 408901 | AK001330 | Hs.48855 | hypothetical protein FLJ10468 | 5.33 | 316 4808 |
| 447836 | F11364 | | gb: HSC2VH051 normalized infant brain cDN | 5.29 | 3912 |
| 442790 | AW663221 | Hs.159057 | ESTs | 5.27 | 3470 |
| 408310 | AW179023 | Hs.191705 | gb: PM3-ST0036-170899-001-e08 ST0036 Homo | 5.27 | 254 |
| 407792 | AI077715 | Hs.39384 | putative secreted ligand homologous to f | 5.26 | 196 |
| 413597 | AW302885 | Hs.117183 | ESTs | 5.26 | 739 |
| 430636 | Z83737 | Hs.247696 | H3 histone family, member J | 5.23 | 2500 5460 |
| 437673 | AW665665 | Hs.153034 | ESTs | 5.22 | 3081 |
| 413278 | BE563085 | Hs.833 | interferon-stimulated protein, 15 kDa | 5.20 | 705 |
| 418004 | U37519 | Hs.87539 | aldehyde dehydrogenase 3 family, member | 5.19 | 1145 4997 |
| 402990 | | | Target Exon | 5.19 | |
| 450278 | AW205234 | Hs.201587 | ESTs | 5.18 | 4123 |
| 411678 | AI907114 | Hs.71465 | squalene epoxidase | 5.16 | 568 |
| 453160 | AI263307 | | H2B histone family, member L | 5.16 | 4380 |
| 444734 | NM_001360 | Hs.11806 | 7-dehydrocholesterol reductase | 5.15 | 3622 5644 |
| 429451 | BE409861 | Hs.202833 | heme oxygenase (decycling) 1 | 5.14 | 2372 |
| 405501 | | | Target Exon | 5.14 | |
| 427560 | AA405394 | Hs.161851 | ESTs | 5.13 | 2166 |
| 428301 | AW628666 | Hs.98440 | ESTs, Weakly similar to I38022 hypotheti | 5.13 | 2238 |
| 402586 | | | ENSP00000241318*: DJ947L8.1.1 (novel CUB | 5.13 | |
| 428566 | U41763 | Hs.184916 | clathrin, heavy polypeptide-like 1 | 5.12 | 2271 5365 |
| 401612 | | | C4000495: gi\|6677633\|ref\|NP_033595.1\|zin | 5.11 | |
| 404120 | | | C5000537*: gi\|3298595\|gb\|AAC41376.1\| (AF0 | 5.10 | |
| 405850 | | | Target Exon | 5.10 | |
| 433055 | AF073727 | Hs.279953 | EH domain-binding mitotic phosphoprotein | 5.09 | 2729 |
| 405210 | | | ENSP00000244302*: CDNA FLJ11591 fis, clon | 5.07 | |
| 455416 | AW937143 | | gb: PM1-DT0041-281299-001-f01 DT0041 Homo | 5.07 | 4507 |
| 428182 | BE386042 | Hs.293317 | ESTs, Weakly similar to GGC1_HUMAN G ANT | 5.05 | 2227 |
| 407239 | AA076350 | Hs.67846 | leukocyte immunoglobulin-like receptor, | 5.03 | 141 |
| 449162 | AI632740 | Hs.10476 | ESTs | 5.02 | 4041 |
| 434203 | BE262677 | Hs.283558 | hypothetical protein PRO1855 | 5.02 | 2820 |
| 434567 | AK000600 | Hs.3972 | NeuAc-alpha-2,3-Gal-beta-1,3-GalNAc-alph | 5.02 | 2848 5550 |
| 433113 | AA622130 | Hs.152524 | ESTs, Weakly similar to PGCB MOUSE BREVI | 5.00 | 2736 |
| 432504 | AL121015 | Hs.277704 | oxygen regulated protein (150 kD) | 4.99 | 2675 |
| 431667 | AA812573 | Hs.246787 | ESTs | 4.99 | 2581 |
| 428970 | BE276891 | Hs.194691 | retinoic acid induced 3 (RAIG1); metabo | 4.99 | 2321 |
| 416700 | AW498958 | Hs.343475 | cathepsin D (lysosomal aspartyl protease | 4.99 | 1014 |
| 446230 | AA134486 | Hs.7155 | ESTs, Moderately similar to 2115357A TYK | 4.98 | 3737 |
| 434637 | AF147432 | Hs.176926 | ESTs | 4.97 | 2857 |
| 453857 | AL080235 | Hs.35861 | Ras-induced senescence 1 (RIS1) | 4.97 | 4437 5785 |
| 429438 | AC006293 | | Homo sapiens killer cell lg-like recepto | 4.95 | 2369 5406 |
| 414222 | AL135173 | Hs.878 | sorbitol dehydrogenase | 4.95 | 805 |
| 423541 | AA296922 | Hs.129778 | serine protease inhibitor, Kazal type 4 | 4.94 | 1756 |
| 422095 | AI868872 | Hs.282804 | hypothetical protein FLJ22704 | 4.93 | 1603 |
| 453885 | AW002629 | Hs.259220 | ESTs | 4.93 | 4445 |
| 426006 | R49031 | Hs.22627 | ESTs | 4.91 | 2019 |
| 405979 | | | Target Exon | 4.91 | |
| 433352 | AA376773 | | gb: EST89237 Small intestine I Homo sapie | 4.90 | 2758 |
| 453922 | AF053306 | Hs.36708 | budding uninhibited by benzimidazoles 1 | 4.90 | 4452 5789 |
| 447388 | AW630534 | Hs.76277 | Homo sapiens, clone MGC: 9381, mRNA, comp | 4.88 | 3854 |
| 412988 | BE046680 | | gb: hn42h03.x1 NCI_CGAP_RDF2 Homo sapiens | 4.88 | |
| 409310 | R88721 | Hs.164584 | ESTs | 4.88 | 360 |
| 436481 | AA379597 | Hs.5199 | HSPC150 protein similar to ubiquitin-con | 4.87 | 2988 |
| 406685 | M18728 | | gb: Human nonspecific crossreacting antig | 4.87 | 4745 83 |
| 429294 | AA095971 | Hs.198793 | Homo sapiens cDNA: FLJ22463 fis, clone H | 4.86 | 2351 |
| 418675 | AW299723 | Hs.87223 | bone morphogenetic protein receptor, typ | 4.86 | 1225 |
| 427715 | BE245274 | Hs.180428 | KIAA1181 protein | 4.86 | 2188 |
| 425443 | AW157547 | Hs.115329 | ESTs | 4.85 | 1964 |
| 402885 | | | Target Exon | 4.84 | |
| 407704 | BE315072 | Hs.78768 | malignant cell expression-enhanced gene/ | 4.84 | 184 |
| 437352 | AL353957 | Hs.284181 | hypothetical protein DKFZp434P0531 | 4.83 | 3060 5585 |
| 404790 | | | C12001707*: gi\|7305215\|ref\|NP_038599.1\|k | 4.82 | |
| 427747 | AW411425 | Hs.180655 | serine/threonine kinase 12 | 4.80 | 2193 |
| 450149 | AW969781 | Hs.132863 | Zic family member 2 (odd-paired Drosophi | 4.80 | 4106 |
| 412519 | AA196241 | Hs.73980 | troponin T1, skeletal, slow | 4.79 | 641 |
| 401281 | | | DKFZP586N2124 protein | 4.78 | |
| 432969 | AA780472 | Hs.335557 | ESTs | 4.78 | 2716 |
| 445075 | AI651827 | | ESTs | 4.78 | 3649 |
| 406981 | S71129 | | acetylcholinesterase (YT blood group) | 4.78 | 117 4761 |
| 409162 | H25530 | Hs.50868 | solute carrier family 22 (organic cation | 4.77 | 343 |
| 428405 | Y00762 | Hs.2266 | cholinergic receptor, nicotinic, alpha p | 4.77 | 2250 5357 |
| 452838 | U65011 | Hs.30743 | preferentially expressed antigen in mela | 4.76 | 4353 5765 |
| 453005 | AW055308 | Hs.31803 | ESTs, Weakly similar to N-WASP H. sapien | 4.76 | 4365 |
| 418203 | X54942 | Hs.83758 | CDC28 protein kinase 2 | 4.75 | 1169 5003 |
| 453712 | AW403791 | Hs.55067 | hypothetical protein MGC15437 | 4.75 | 4426 |
| 406718 | AA505525 | Hs.169476 | glyceraldehyde-3-phosphate dehydrogenase | 4.74 | 94 |
| 411598 | BE336654 | Hs.70937 | H3 histone family, member A | 4.73 | 562 |
| 431882 | NM_001426 | Hs.271977 | engrailed homolog 1 | 4.73 | 2612 5493 |
| 443219 | AI354669 | Hs.187461 | ESTs, Weakly similar to C29149 proline-r | 4.73 | 3509 |
| 438956 | W00847 | Hs.135056 | Human DNA sequence from clone RP5-850E9 | 4.73 | 3180 |
| 418960 | NM_004494 | Hs.89525 | hepatoma-derived growth factor (HDGF) | 4.72 | 1263 5034 |
| 457739 | AF161337 | Hs.283928 | Homo sapiens HSPC074 mRNA, partial cds | 4.71 | 4600 5815 |
| 412999 | BE046255 | | gb: hn38g10.x2 NCI_CGAP_RDF2 Homo sapiens | 4.70 | |
| 420856 | BE513294 | Hs.205736 | HLA class II region expressed gene KE2 | 4.69 | 1461 |
| 408633 | AW963372 | Hs.46677 | PRO2000 protein | 4.69 | 286 |
| 435602 | AF217515 | Hs.283532 | uncharacterized bone marrow protein BM03 | 4.69 | 2931 5567 |
| 435493 | AW135312 | Hs.117237 | ESTs | 4.68 | 2920 |
| 409469 | AW517236 | Hs.318393 | ESTs | 4.68 | 377 |
| 407137 | T97307 | | gb: ye53h05.s1 Soares fetal liver spleen | 4.67 | 128 |
| 444441 | AW613841 | Hs.301394 | hypothetical protein MGC3101 | 4.67 | 3598 |
| 410959 | AW811873 | | gb: RC2-ST0168-071299-013-h10 ST0168 Homo | 4.67 | 523 |
| 402504 | | | C1003823*: gi\|4826521\|emb\|CAB42853.1\| (AL | 4.67 | |
| 417037 | BE083936 | Hs.80976 | antigen identified by monoclonal antibod | 4.66 | 1056 |
| 433399 | N46406 | Hs.84700 | similar to phosphatidylcholine transfer | 4.65 | 2760 |
| 436057 | AJ004832 | Hs.5038 | neuropathy target esterase | 4.65 | 2960 5575 |
| 409034 | AI684149 | Hs.172035 | hypothetical protein similar to mouse HN | 4.65 | 325 |
| 422830 | AC007954 | Hs.121371 | hypothetical protein DKFZp434P0111 | 4.64 | 1685 5159 |
| 421975 | AW961017 | Hs.6459 | hypothetical protein FLJ11856 | 4.64 | 1587 |
| 419760 | AA668227 | Hs.316625 | gb: ab77e07.s1 Stratagene fetal retina 93 | 4.63 | 1359 |
| 400657 | | | Target Exon | 4.63 | |
| 425003 | AF119046 | Hs.154149 | apurinic/apyrimidinic endonuclease(APEX | 4.61 | 1909 5242 |
| 432241 | AI937060 | Hs.6298 | KIAA1151 protein | 4.61 | 2648 |
| 459010 | AA331438 | | gb: EST35269 Embryo, 8 week I Homo sapien | 4.61 | 4652 |
| 438577 | AA811184 | Hs.259785 | carnitine palmitoyltransferase I, liver | 4.61 | 3150 |
| 441593 | AA939228 | Hs.163412 | ESTs | 4.61 | 3359 |
| 440238 | AW451970 | Hs.155644 | paired box gene 2 | 4.61 | 3273 |
| 429883 | AI125209 | Hs.123848 | ESTs | 4.59 | 2421 |
| 428500 | AI815395 | Hs.184641 | fatty acid desaturase 2 | 4.59 | 2266 |
| 450029 | AW073380 | Hs.267963 | hypothetical protein FLJ10535 | 4.58 | 4098 |
| 436608 | AA628980 | Hs.192371 | down syndrome critical region protein DS | 4.58 | 2997 |
| 411263 | BE297802 | Hs.69360 | kinesin-like 6 (mitotic centromere-assoc | 4.58 | 544 |
| 403156 | | | C2001591: gi\|10566471\|dbj\|BAB15806.1\| (AB | 4.58 | |
| 437026 | AW976573 | | ESTs | 4.57 | 3029 |
| 403481 | | | Target Exon | 4.57 | |
| 432886 | BE159028 | Hs.279704 | chromatin accessibility complex 1 | 4.56 | 2708 |
| 450935 | BE514743 | | tumor suppressor deleted in oral cancer- | 4.55 | 4176 |
| 454425 | AW300927 | Hs.27192 | hypothetical protein dJ1057B20.2 | 4.55 | 4482 |
| 423306 | W88562 | Hs.108198 | ESTs | 4.55 | 1735 |
| 442505 | AW003775 | Hs.343822 | UDP-Gal: betaGlcNAc beta 1,4-galactosylt | 4.54 | 3436 |
| 429345 | R11141 | Hs.199695 | hypothetical protein | 4.53 | 2357 |
| 407300 | AA102616 | Hs.120769 | gb: zn43e07.s1 Stratagene HeLa cell s3 93 | 4.53 | 149 |
| 405333 | | | Target Exon | 4.53 | |
| 402808 | | | ENSP00000235229: SEMB. | 4.53 | |
| 429746 | AJ237672 | Hs.214142 | 5,10-methylenetetrahydrofolate reductase | 4.53 | 2412 5424 |
| 448604 | AI546830 | | gb: PN2.1_07_D12.r mynom, Homo sapiens cD | 4.52 | 3988 |
| 410156 | AA082005 | Hs.9634 | ESTs | 4.52 | 450 |
| 418216 | AA662240 | Hs.283099 | AF15q14 protein | 4.51 | 1171 |
| 459358 | T24769 | Hs.343627 | hypothetical protein FLJ12998 | 4.50 | 4666 |
| 426516 | BE262660 | Hs.170197 | glutamic-oxaloacetic transaminase 2, mit | 4.50 | 2074 |
| 440061 | BE396581 | | gb: 601288812F1 NIH_MGC_8 Homo sapiens CD | 4.50 | 3258 |
| 422997 | BE018212 | Hs.122908 | DNA replication factor | 4.50 | 1708 |
| 407376 | AA993138 | Hs.142287 | ESTs, Weakly similar to ALUF_HUMAN !!!! | 4.49 | 159 |
| 408877 | AA479033 | Hs.130315 | ESTs, Weakly similar to A47582 B-cell gr | 4.48 | 314 |
| 449256 | AA059050 | Hs.59847 | ESTs | 4.47 | 4051 |
| 428227 | AA321649 | Hs.2248 | small inducible cytokine subfamily B (Cy | 4.46 | 2231 |
| 439273 | AW139099 | Hs.269701 | ESTs | 4.46 | 3195 |
| 418758 | AW959311 | Hs.172012 | hypothetical protein DKFZp434J037 | 4.46 | 1237 |
| 417158 | AW965223 | Hs.110062 | complement-c1q tumor necrosis factor-rel | 4.45 | 1065 |
| 404157 | | | C6001170: gi\|6468391\|emb\|CAB61578.1\| (AL0 | 4.45 | |
| 421777 | BE562088 | Hs.108196 | HSPC037 protein | 4.45 | 1562 |
| 416555 | H63394 | Hs.334792 | ESTs | 4.44 | 1003 |
| 454422 | AW072328 | Hs.59728 | Homo sapiens mRNA; cDNA DKFZp566C0546 (f | 4.44 | 4481 |
| 442432 | BE093589 | Hs.38178 | hypothetical protein FLJ23468 | 4.44 | 3429 |
| 444118 | AA458542 | Hs.10326 | coatomer protein complex, subunit epsilo | 4.44 | 3576 |
| 407561 | BE313226 | Hs.94761 | KIAA1691 protein | 4.44 | 167 |
| 405101 | | | ENSP00000249234*: Zinc finger protein 92 | 4.43 | |
| 459709 | AA653774 | Hs.212084 | ESTs | 4.43 | 4679 |
| 452833 | BE559681 | Hs.30736 | KIAA0124 protein | 4.43 | 4351 |
| 440087 | W28969 | Hs.7718 | hypothetical protein FLJ22678 | 4.43 | 3260 |
| 453833 | AF090320 | Hs.35718 | cytochrome P450, subfamily VIIIB (sterol | 4.43 | 4435 5784 |
| 433145 | BE256247 | Hs.7740 | oxysterol binding protein-like 1 | 4.43 | 2739 |
| 408771 | AW732573 | Hs.47584 | potassium voltage-gated channel, delayed | 4.42 | 305 |
| 430820 | AF194815 | | immunoglobulin lambda variable 4-3 | 4.42 | 2516 5463 |
| 458756 | AW057910 | Hs.282185 | ESTs | 4.42 | 4636 |
| 432415 | T16971 | Hs.289014 | ESTs, Weakly similar to A43932 mucin 2 p | 4.41 | 2665 |
| 433001 | AF217513 | Hs.279905 | clone HQ0310 PRO0310p1 | 4.40 | 2719 5521 |
| 426691 | NM_006201 | Hs.171834 | PCTAIRE protein kinase 1 | 4.40 | 2088 5314 |
| 435579 | AI332373 | Hs.156924 | ESTs | 4.40 | 2930 |
| 402299 | | | Target Exon | 4.40 | |
| 439926 | AW014875 | Hs.137007 | ESTs | 4.40 | 3243 |
| 413762 | AW411479 | Hs.848 | FK506-binding protein 4 (59 kD) | 4.39 | 751 |
| 406181 | | | Target Exon | 4.39 | |
| 458098 | BE550224 | | metallothionein 1E (functional) | 4.38 | 4611 |
| 405573 | | | Target Exon | 4.37 | |
| 434761 | AW298777 | Hs.192155 | ESTs | 4.37 | 2864 |
| 424411 | NM_005209 | Hs.146549 | crystallin, beta A2 | 4.36 | 1841 5211 |
| 451827 | BE387187 | Hs.27184 | growth factor, erv1 (S. cerevisiae)-like | 4.36 | 4252 |
| 445013 | AF151022 | Hs.300224 | hypothetical protein | 4.36 | 3646 5649 |
| 446439 | D87437 | Hs.15087 | KIAA0250 gene product | 4.35 | 3750 5667 |
| 456604 | AW383770 | Hs.131878 | ESTs, Highly similar to S22745 serine/th | 4.35 | 4554 |
| 414423 | BE045599 | Hs.202612 | ESTs | 4.35 | 825 |
| 439602 | W79114 | Hs.58558 | ESTs | 4.35 | 3222 |
| 427584 | BE410293 | Hs.179718 | v-myb avian myeloblastosis viral oncogen | 4.35 | 2168 |
| 400530 | | | Target Exon | 4.35 | |
| 420352 | BE258835 | Hs.347540 | gb: 601117374F1 NIH_MGC_16 Homo sapiens c | 4.34 | 1416 |
| 402229 | | | mitochondrial ribosomal protein S2 | 4.33 | |
| 409902 | AI337658 | Hs.156351 | ESTs | 4.33 | 416 |
| 452012 | AA307703 | Hs.279766 | kinesin family member 4A | 4.33 | 4262 |
| 409557 | BE182896 | Hs.3686 | ESTs | 4.33 | 384 |
| 452092 | BE245374 | Hs.27842 | hypothetical protein FLJ11210 | 4.32 | 4268 |
| 456623 | AI084125 | Hs.108106 | transcription factor | 4.32 | 4555 |
| 430361 | AI033965 | Hs.239926 | sterol-C4-methyl oxidase-like | 4.31 | 2476 |
| 437150 | R51407 | Hs.77910 | 3-hydroxy-3-methylglutaryl-Coenzyme A sy | 4.31 | 3038 |
| 428619 | AK002140 | Hs.187378 | hypothetical protein FLJ11278 | 4.31 | 2277 5368 |
| 426902 | AI125334 | Hs.97408 | ESTs | 4.30 | 2110 |
| 421994 | BE542166 | | ESTs, Weakly similar to I78885 serine/th | 4.30 | 1590 |
| 407539 | X91103 | | gb: H. sapiens mRNA for Hr44 protein. | 4.30 | 166 4774 |
| 428977 | AK001404 | Hs.194698 | cyclin B2 | 4.29 | 2323 |
| 410348 | AW182663 | Hs.95469 | ESTs | 4.29 | 467 |
| 406355 | | | C5000598: gi\|2136258\|pir\|\|I59377 template | 4.28 | |
| 454033 | AF107457 | Hs.37035 | homeo box HB9 | 4.28 | 4461 |
| 444893 | AW249312 | Hs.12109 | WD40 protein Ciao1 | 4.28 | 3637 |
| 424796 | AW298244 | Hs.266195 | ESTs | 4.28 | 1887 |
| 410009 | AA079555 | Hs.146092 | ESTs | 4.28 | 428 |
| 401807 | | | C7001350: gi\|6578126\|gb\|AAF17706.1\|AF0496 | 4.28 | |
| 403347 | | | Target Exon | 4.27 | |
| 448296 | BE622756 | Hs.10949 | Homo sapiens cDNA FLJ14162 fis, clone NT | 4.26 | 3956 |
| 426613 | U96132 | Hs.171280 | hydroxyacyl-Coenzyme A dehydrogenase, ty | 4.26 | 2083 5313 |
| 447987 | BE621544 | Hs.157160 | hypothetical protein MGC2616 | 4.26 | 3932 |
| 406043 | | | Target Exon | 4.25 | |
| 439453 | BE264974 | Hs.6566 | thyroid hormone receptor interactor 13 | 4.25 | 3208 |
| 417207 | N92226 | Hs.338218 | ESTs | 4.25 | 1072 |
| 428971 | BE278404 | Hs.285813 | hypothetical protein FLJ11807 | 4.25 | 2322 |
| 404816 | | | ENSP00000251989*: DJ100N22.1 (NOVEL EGF-L | 4.25 | |
| 425662 | BE173463 | | proliferation-associated 2G4, 38 kD | 4.25 | 1983 |
| 419409 | AW297831 | Hs.143792 | hypothetical protein MGC2656 | 4.24 | 1312 |
| 458744 | AW445183 | | ESTs | 4.24 | 4634 |
| 452461 | N78223 | Hs.108106 | transcription factor | 4.24 | 4311 |
| 436241 | AI051175 | Hs.119594 | ESTs | 4.23 | 2970 |
| 404068 | | | Target Exon | 4.23 | |
| 441362 | BE614410 | Hs.23044 | RAD51 (S. cerevisiae) homolog (E coli Re | 4.22 | 3347 |
| 442916 | H15560 | Hs.131833 | ESTs | 4.22 | 3481 |
| 418897 | AW016578 | Hs.128630 | ESTs | 4.22 | 1256 |
| 410211 | NM_014347 | Hs.296365 | zinc finger protein | 4.22 | 456 4841 |
| 426989 | AI815206 | | ESTs | 4.21 | 2115 |
| 425184 | BE278288 | Hs.155048 | Lutheran blood group (Auberger b antigen | 4.20 | 1934 |
| 409757 | NM_001898 | Hs.123114 | cystatin SN | 4.20 | 403 4832 |
| 424991 | AA775471 | Hs.241467 | ESTs | 4.20 | 1904 |
| 438792 | AW291313 | Hs.254955 | ESTs | 4.20 | 3165 |
| 452369 | AA766459 | | gb: oa32f07.s1 NCI_CGAP_GCB1 Homo sapiens | 4.20 | 4301 |
| 429873 | AW268693 | Hs.105713 | ESTs | 4.20 | 2420 |
| 454171 | AW854832 | | gb: QV2-CT0261-201099-011-f05 CT0261 Homo | 4.20 | |
| 418317 | R59783 | | gb: yh07f05.r1 Soares infant brain 1NIB H | 4.19 | 1181 |
| 414362 | AI347934 | Hs.75932 | N-ethylmaleimide-sensitive factor attach | 4.19 | 817 |
| 431851 | AI146349 | Hs.271614 | CGI-112 protein | 4.19 | 2608 |
| 457485 | AW081072 | Hs.115960 | KIAA0939 protein | 4.18 | 4593 |
| 446342 | BE298665 | Hs.14846 | solute carrier family 7 (cationic amino | 4.18 | 3746 |
| 421254 | AK001724 | Hs.102950 | coat protein gamma-cop | 4.18 | 1496 |
| 432738 | AI559493 | Hs.165904 | ESTs | 4.17 | 2697 |
| 406216 | | | Target Exon | 4.17 | |
| 459679 | AA936176 | Hs.303666 | gb: om06b10.s1 Soares_NFL_T_GBC_S1 Homo s | 4.17 | 4677 |
| 420996 | AK001927 | Hs.100895 | hypothetical protein FLJ10462 | 4.17 | 1473 5092 |
| 434225 | AA627706 | | ESTs | 4.17 | 2822 |
| 416920 | AA176455 | Hs.80475 | polymerase (RNA) II (DNA directed) polyp | 4.16 | 1037 |
| 441378 | AA931826 | Hs.126846 | ESTs | 4.16 | 3350 |
| 437848 | AI906419 | Hs.284380 | gamma-glutamyltransferase 1 | 4.15 | 3098 |
| 442041 | AW161137 | Hs.209569 | ESTs | 4.15 | 3389 |
| 458176 | AI961519 | Hs.140309 | Homo sapiens, clone IMAGE: 3677194, mRNA, | 4.15 | 4613 |
| 428732 | AA432266 | | ESTs | 4.15 | 2289 |
| 440205 | T86950 | Hs.105448 | ESTs, Weakly similar to B34087 hypotheti | 4.15 | 3271 |
| 456341 | AA229126 | | gb: nc45e10.s1 NCI_CGAP_Pr3 Homo sapiens | 4.15 | 4537 |
| 408946 | AW854991 | Hs.255565 | ESTs | 4.15 | 318 |
| 417923 | R26632 | Hs.106510 | ESTs, Moderately similar to ALU2_HUMAN A | 4.14 | 1137 |
| 419092 | J05581 | Hs.89603 | mucin 1, transmembrane | 4.13 | 1275 5038 |
| 415228 | AF030111 | Hs.78281 | regulator of G-protein signalling 12 | 4.13 | 4943 918 |
| 453377 | AI679149 | Hs.167186 | ESTs | 4.13 | 4402 |
| 443361 | AI792628 | Hs.133273 | ESTs | 4.13 | 3520 |
| 425453 | AW374284 | Hs.237617 | Homo sapiens chromosome 19, cosmid R2689 | 4.13 | 1966 |
| 437933 | AI276132 | | ESTs | 4.12 | 3109 |
| 448484 | BE613340 | Hs.334725 | Homo sapiens, Similar to RIKEN cDNA 9430 | 4.11 | 3975 |
| 431629 | AU077025 | Hs.265827 | interferon, alpha-inducible protein (clo | 4.11 | 2577 |
| 416138 | C18946 | Hs.79026 | myeloid leukemia factor 2 | 4.11 | 976 |
| 417866 | AW067903 | Hs.82772 | collagen, type Xl, alpha 1 | 4.11 | 1132 |
| 408349 | BE546947 | Hs.44276 | homeo box C10 | 4.10 | 258 |
| 405945 | | | Target Exon | 4.10 | |
| 431611 | U58766 | Hs.264428 | tissue specific transplantation antigen | 4.10 | 2575 5482 |
| 429485 | AW197086 | Hs.99338 | ESTs | 4.10 | 2375 |
| 456847 | AI360456 | Hs.37776 | ESTs | 4.10 | 4563 |
| 435043 | AI276478 | | ESTs | 4.10 | 2890 |
| 413976 | BE295452 | Hs.75655 | procollagen-proline, 2-oxoglutarate 4-di | 4.09 | 771 |
| 431374 | BE258532 | Hs.251871 | CTP synthase | 4.09 | 2551 |
| 419102 | AA234098 | Hs.42424 | ESTs, Weakly similar to 2004399A chromos | 4.09 | 1276 |
| 453863 | X02544 | Hs.572 | orosomucoid 1 | 4.09 | 4438 5786 |
| 404755 | | | Target Exon | 4.08 | |
| 448633 | AA311426 | Hs.21635 | tubulin, gamma 1 | 4.08 | 3990 |
| 418478 | U38945 | Hs.1174 | cyclin-dependent kinase inhibitor 2A (me | 4.08 | 1204 5017 |
| 425966 | NM_001761 | Hs.1973 | cyclin F | 4.08 | 2015 5284 |
| 409929 | R38772 | Hs.172619 | myelin transcription factor 1-like | 4.08 | 419 |
| 440042 | AI073387 | Hs.133898 | ESTs | 4.08 | 3255 |
| 437679 | NM_014214 | Hs.5753 | inositol(myo)-1(or 4)-monophosphatase 2 | 4.07 | 3082 5590 |
| 447995 | AI742618 | Hs.181733 | ESTs, Weakly similar to nitrilase homolo | 4.07 | 3934 |
| 417059 | AL037672 | Hs.81071 | extracellular matrix protein 1 | 4.07 | 1059 |
| 427719 | AI393122 | Hs.134726 | ESTs | 4.07 | 2189 |
| 431912 | AI660552 | | ESTs, Weakly similar to A56154 Abl subst | 4.07 | 2615 |
| 411377 | AW841462 | | gb: RC6-CN0014-080300-012-B09 CN0014 Homo | 4.07 | 549 |
| 418205 | L21715 | Hs.83760 | troponin I, skeletal, fast | 4.06 | 1170 5004 |
| 444806 | AI197853 | Hs.312841 | ESTs | 4.05 | 3630 |
| 455513 | AW983772 | | gb: RC3-HN0002-060400-012-h09 HN0002 Homo | 4.05 | 4513 |
| 407487 | S70348 | | gb: Homo sapiens integrin beta 3 mRNA, pa | 4.05 | 164 4772 |
| 451365 | AI791783 | Hs.16063 | ESTs, Weakly similar to ALU8_HUMAN ALU S | 4.05 | 4215 |
| 420848 | NM_005188 | Hs.99980 | Cas-Br-M (murine) ecotropic retroviral t | 4.05 | 1459 5090 |
| 442739 | NM_007274 | Hs.8679 | cytosolic acyl coenzyme A thioester hydr | 4.05 | 3464 5626 |
| 445871 | AI702901 | Hs.145582 | ESTs, Weakly similar to FOR4 MOUSE FORMI | 4.05 | 3706 |
| 412007 | AA999846 | Hs.71986 | ESTs | 4.05 | 602 |
| 459682 | AW241940 | Hs.102500 | hypothetical protein FLJ20481 | 4.04 | 4678 |
| 425234 | AW152225 | Hs.165909 | ESTs, Weakly similar to I38022 hypotheti | 4.04 | 1940 |
| 410082 | AA081594 | Hs.158311 | Musashi (Drosophila) homolog 1 | 4.04 | 442 |
| 409776 | AW499663 | | gb: UI-HF-BR0p-ajj-d-03-0-UI.r1 NIH_MGC_5 | 4.04 | 405 |
| 452533 | AW967801 | Hs.151293 | ESTs, Weakly similar to T42705 hypotheti | 4.04 | 4323 |
| 407673 | AW064404 | Hs.279825 | ESTs | 4.03 | 179 |
| 426609 | AL040604 | Hs.99344 | hypothetical protein PRTD-NY3 | 4.03 | 2082 |
| 422010 | AA302049 | Hs.31181 | Homo sapiens cDNA: FLJ23230 fis, clone C | 4.02 | 1593 |
| 453435 | AJ278473 | Hs.297814 | gb: AJ278473 Homo sapiens adult T-cell le | 4.02 | 4404 |
| 409844 | AW502336 | | gb: UI-HF-BR0p-aka-b-05-0-UI.r1 NIH_MGC_5 | 4.02 | 409 |
| 433294 | AA582082 | Hs.199410 | ESTs | 4.02 | 2754 |
| 420415 | L44281 | Hs.73149 | paired box gene 8 | 4.02 | 1419 |
| 406253 | | | Target Exon | 4.02 | |
| 414602 | AW630088 | Hs.76550 | Homo sapiens mRNA; cDNA DKFZp564B1264 (f | 4.01 | 853 |
| 425081 | X74794 | Hs.154443 | minichromosome maintenance deficient (S. | 4.01 | 1918 5246 |
| 452613 | AA461599 | Hs.23459 | ESTs | 4.01 | 4329 |
| 454831 | AW833768 | | gb: QV4-TT0008-130100-077-e06 TT0008 Homo | 4.00 | 4496 |
| 443147 | AI034351 | Hs.19030 | ESTs | 4.00 | 3499 |
| 414571 | BE410746 | Hs.22868 | protein tyrosine phosphatase, non-recept | 4.00 | 846 |
| 447316 | AI373534 | Hs.337577 | ESTs | 4.00 | 3841 |

Pkey: Unique Eos probeset identifier number
ExAccn: Exemplar Accession number, Genbank accession number
UniGeneID: UniGene number
UniGene Title: UniGene gene title
R1: 90th percentile of breast metastases to the brain Als divided by the 90th percentile of normal breast tissue Als, where the 15th percentile of all normal body tissue Als was subtracted from the both numerator and denominator.
SEQ ID NO(s): SEQ ID number(s) for nucleic acid and protein sequences associated with table entry.

TABLE 1B

| Pkey | CAT number | Accession |
|---|---|---|
| 400205 | 2538_1 | NM_006265 D38551 X98294 BM477931 BM461566 AU123557 AU133303 AU134649 AW500421 BM172439 AW500587 AW503665 |
| | | AW504355 AW503640 BM152454 AW505260 AI815984 AW504075 AW500716 AL597310 BC001229 BM474371 AA984202 AU135205 |
| | | BE090841 AW163750 BF747730 BF898637 AI206506 AV660870 AV692110 AW386830 AV656831 N84710 AW993470 BF086802 |
| | | BF758454 BG960772 BF757769 BI870853 BE018627 C75436 AW148744 BF757753 BG622067 BE909924 AA708208 BG530266 BF968015 |
| | | AW992930 BF888862 BG536628 AA143164 AW748953 BG498922 BF885190 BF889005 BF754781 BF800003 BM476529 AI627668 |
| | | AW028126 AL046011 BF590668 AI017447 AA579936 AI367597 AA699622 BE280597 AI124620 AI082548 AW274985 AA677870 AI056767 |
| | | BE551689 AA287642 H94499 AI752427 AI652365 AW002374 AW062651 AA360834 N68822 AU135442 AU125960 Z78334 BE545813 |
| | | AI092115 BF312771 BF242859 BG533616 BG533761 BG164745 BG492433 BM473183 AA172043 AA172069 AU157092 AU151353 |
| | | AU155318 BE302211 AI375022 AA085641 AU157923 H88858 AA132730 AA115113 AA909781 AI475256 AA424206 AW572383 AW084296 |
| | | AI184820 AI469178 AA782432 H92184 AA340562 BF195818 AA852821 AW576342 AA827107 AA173317 AW190014 AI918514 AA729372 |
| | | AA729718 AI055958 AA331424 BE328601 AA515690 BI018896 AW628277 AA748368 AA626222 BG492636 AW380620 BF800058 |
| | | AW370956 AA290909 R25857 BG952995 BF801437 AA172077 AU155890 AU149783 AI720904 AA902936 AA865727 AI470830 AV740677 |
| | | AA142982 AA482485 AU145485 AW576399 AU156042 R63448 BF246427 BE928472 D25910 BF758439 BF968785 BE565238 AA355981 |
| | | AI905607 BG291148 BG533096 BG532888 BF030886 BG613756 BE928471 BG574501 AA187596 AA361196 T95557 BG531446 |
| | | BG527242 BG527513 BG611106 AA085995 BF847252 BG024608 BE540261 BG531236 AL579993 BG108733 BG483503 BG571032 |
| | | BG492505 |
| 400183 | 48154_3 | X80199 NM_007359 BI553784 BG720095 BG899766 AI088926 BE857470 AI199713 AI870291 AL121179 AL577898 AA009896 AA868181 |
| | | AA482924 AI637524 AW043834 AU156777 T30547 R05481 BE902481 AW372677 W73547 H51965 BF944628 BE825156 AL567238 |
| | | AW372714 BF759809 AL578496 BE396176 H02384 BE007647 BF917244 |
| 421582 | 13358_1 | X00474 NM_003225 X52003 M12075 BI765761 AW950155 AI571948 BI760569 AA308400 AA568312 BI761955 AA507595 AA614579 |
| | | AA614409 BF747698 BM142326 AA307578 AI925552 AA578674 AA582084 AW009769 AA514776 AA588034 BG271505 AA858276 |
| | | BM142503 AW050700 AI307407 AI202532 AA524242 AI909772 AI970839 BG236516 AW750216 AA587613 AI909749 AI909751 AI910083 AA614539 R55292 AA507418 |
| 427239 | 20459_2 | AL532360 BE794750 AA582906 AI015067 AW271034 BG271636 AW075177 AW071374 AI345565 AI307208 BE138953 BE049086 |
| | | AI334881 AW075006 AW075181 AA464019 AW302733 AW075100 AW073433 AI802854 AI334909 AI802853 AI345036 AI348921 AI340734 |
| | | AI307478 AI251289 AW302327 AW072520 AI312145 AW073656 AW072513 AW071289 AI307559 AA876186 T29587 AI307493 AI255068 |
| | | AI252868 AI252839 AW074809 AI252926 AI252160 AI251662 AI251262 AI610913 AI270787 AI270156 AI252075 AW073469 AW072901 |
| | | AW072496 AW071420 AI305762 AI254764 AI802837 AI251264 AW073049 AW071311 AI340643 BE138965 BE138502 AW073456 |
| | | AI334733 AI054335 BE139260 AI054302 AI054060 AI054057 AI053722 AI289711 BE139228 AW470478 AW271039 AW302085 BE041872 |
| | | AI254494 AI271496 AI252427 BF718773 BF718645 AW074866 BE857822 |
| 441761 | 173757_1 | BF796007 AI718138 BI600878 AI222880 AI719648 AI242524 AI242392 AA961560 BF770656 AW275807 |
| 457341 | 703_15 | AW948320 BE181716 AW983981 AA484444 AW948314 |
| 433157 | 38399_2 | AA578671 AI559353 AW769671 AW769665 |
| 400222 | 9287_3 | NM_002082 L16862 BG828886 BE795217 BE904064 BE294526 BE297283 BE394617 BE935127 BE935106 F12351 BG823182 H16710 |
| 441153 | 264480_3 | BE562826 BE378727 |
| 412377 | 1174171_1 | AW947536 AW947523 AW947539 AW947540 AW947541 AW947535 |
| 447836 | 570540_1 | BI086295 F11364 BE620332 AW877701 AW877654 AW877608 AW877766 T08447 BE887463 AW956871 |
| 453160 | 6028_5 | BC009612 NM_003526 BI597616 AV761592 AV760377 AL601008 BI604131 BE645918 BG187760 BG181525 BG210634 BG192999 |
| | | AI263307 AA344186 AW952966 AA033609 AA037562 AA722183 R79452 H70775 BF674991 BE769437 BG007856 AA037483 AW572535 AI143991 AA084581 AA033610 AV742510 |
| | | AV735788 R08336 |
| 455416 | 1164250_1 | AW937150 AW937151 AW937141 AW937174 AW937132 AW937173 AW937170 AW937165 AW937195 AW937145 AW937164 AW937137 |
| | | AW937160 AW937142 AW937171 AW937191 AW937139 AW937143 AW937159 AW937140 AW937163 AW937135 AW937179 AW937156 |
| 429438 | 30460_1 | NA |
| 433352 | 61720_1 | AF024708 AF024696 AA376773 BE927220 BG249309 BF741901 BF927642 BE176914 BF364317 |
| 412988 | 1342150_1 | BE046680 BE046738 BE044958 |
| 406685 | 0_0 | M18728 |
| 445075 | 2823108_1 | AI651827 AI206885 AI699543 |
| 412999 | 1343220_1 | BE046255 BE048611 BE046716 BE046732 BE046273 |
| 410959 | 1065309_1 | AW811937 AW811939 AW811934 AW811873 AW811951 AW811938 AW852485 AW811932 BE144621 |
| 459010 | 84453_1 | AW955786 BF870627 AA251104 AA331438 AA016068 Z19751 |
| 437026 | 1240260_1 | AW976573 AA742335 AA830000 |
| 450935 | 4469_1 | BE349913 BC002850 BC016704 NM_005851 AF089814 AK001498 AL537879 BG754157 AI129659 AI261895 BG397540 BF663515 |
| | | AW517226 BG677982 AI435188 AI453123 AI198380 AA524481 BG981512 AW269638 AI348113 AI095743 AA457108 AW044584 |
| | | AU151602 AI744572 AI078741 AI879729 AI089613 AI568618 BF109806 AW440972 AA401965 AI027227 AI127506 AA434027 BF732901 |
| | | AW264768 AA833667 AL038554 AI568161 AI350340 AA402084 BE677778 AA933621 BF663514 BG763563 AL574521 BG177226 |
| | | BG759860 BE906329 AW161323 AI879350 BF946477 BF766208 AA633194 AL537880 AL038553 BG468205 BG761617 BI762655 |
| | | BG436637 N49458 BI834722 BG397879 BI258799 AW004930 AA902847 AI832036 AW167549 AA743167 AA848017 AA365434 AI819503 |
| | | AA767866 AI582000 N33615 AI950146 AA444029 AA227130 AA041525 AW339842 AA865972 AA838006 AW157822 AI890994 BI771150 |
| | | BI821666 BE279491 |
| 448604 | 2997328_1 | AI546830 AI546838 |
| 440061 | 10031_11 | BG392038 AA090632 AL523849 R14005 BE559751 |
| 430820 | 32425_1 | AF194815 AF194816 |
| 458098 | 23945_1 | AI082245 BE467534 AI797130 BE467063 BE467767 BE218421 AI694996 BE327781 BE327407 BE833829 AA989054 AA459718 BE833855 |
| | | BE550224 AA832519 AF086393 AV733386 BE465409 N29245 W07677 AA482971 BE503548 H18151 AA461301 W79223 W74510 |
| | | AI090689 AL600773 AL600781 N46003 R28075 R34182 BE071550 AW885857 AI276145 AI276696 H97808 N20540 AI468553 |
| 421994 | 864408_1 | BE542166 AI536692 W49486 AI554576 AI359949 AA329007 AA301695 AA887288 AW954743 BI053257 W49487 |
| 425662 | 29204_6 | BF576185 BG392835 BF832725 AA348657 BE173463 H38593 AA361336 |
| 458744 | 827170_1 | AW445183 AI380746 |
| 426989 | 289835_1 | H10322 AW191920 AA581798 BF909366 BF909310 BF909357 BF909323 BF909317 BF909442 BF909364 BF909342 T23471 F02195 |
| | | AA455133 F04743 D19749 AI224921 BF907691 BF909436 BF909433 BF909347 BF908960 BF911433 BF909439 BF909320 BF908633 |
| | | BF909360 BF909344 BF909326 BF909330 BF909324 BF909322 AA394039 BF909349 AA857913 BF909352 BF912020 BF911220 |
| | | BF909363 BF911329 BF909339 BF907711 BF909341 BF912530 BF912434 BF912513 BF912417 BF912223 BF909336 BF909328 |
| | | BF911219 BF909356 BF909337 AI815206 BF912221 R49595 F02092 R44981 BF909318 BF380684 H17049 H05273 BF909312 BF909314 |
| | | BF912225 |
| 452369 | 89607_1 | AW968737 AA766459 AA025799 |
| 454171 | 1049240_1 | AW854832 AW854798 AW854857 AW854816 AW854834 AW854817 |
| 418317 | 1176924_1 | W22626 BF110459 BI020483 BI025592 BI020028 R59783 T24708 |
| 434225 | 1861692_1 | AA627706 F28433 D63189 |
| 428732 | 13917_2 | AW604761 BG496605 AL534435 N52169 Z40534 AL514785 BF834286 BF832372 AA102279 BG260063 AU123387 BG989683 BF887489 |
| | | BG427243 AA757223 N46621 BE774691 BF223203 AI638487 AI685529 AI656376 AA628336 BF431278 N34895 AA705819 AI921735 |
| | | AA432266 BE067482 AW601418 AI684438 AI251741 AI653304 AA595843 AA431254 AI419788 AI220525 AA620860 AA719177 AA860379 |
| | | AW974279 Z47308 AA299472 AY072912 AL514786 Z44690 AA089566 F05145 AI557255 AI678039 AA926992 AY072910 BG718557 |
| | | AU098965 |
| 456341 | 245628_1 | AA229126 AA229463 |
| 437933 | 724922_1 | AI276132 AA772500 AW295806 AI383186 BE698792 BE698799 |
| 435043 | 811_13 | AA662663 AI432402 AI276478 AI675712 |
| 431912 | 610_10 | BI763666 BI517886 BI759051 AI688604 AI660552 BF588523 AW004785 AW295479 BF591117 BF002672 BF064073 AA594441 AI380340 |
| | | AI700219 AI659950 AI688540 AW296326 |
| 411377 | 1079470_1 | BE092475 BE156657 BE156668 AW841462 |
| 455513 | 1242945_1 | AW983772 AW983837 AW983730 AW983769 AW983836 AW983835 |
| 407487 | 56998_1 | S70348 BF593562 BG999385 BG999377 |
| 409776 | 912213_1 | AW499663 AW502643 AW502632 |
| 409844 | 916085_1 | AW502336 AW502339 AW501736 AW501839 |
| 454831 | 1073690_2 | AW833435 AW833533 AW833768 AW833631 AW833557 |

Pkey: Unique Eos probeset identifier number
CAT number: Gene cluster number
Accession: Genbank accession numbers

TABLE 1C

| Pkey | Ref | Strand | Nt_position |
|---|---|---|---|
| 405452 | 7656638 | Minus | 93876-94275 |
| 400914 | 3779013 | Plus | 116586-116729, 117860-117986 |
| 403532 | 8076842 | Minus | 81750-81901 |
| 402542 | 9801558 | Minus | 67076-67594 |
| 401451 | 6634068 | Minus | 119926-121272 |
| 402265 | 3287673 | Plus | 21059-21168 |
| 403291 | 7230870 | Plus | 95177-95435 |
| 401076 | 3687273 | Plus | 85725-85917 |
| 402907 | 6706902 | Minus | 8561-8692 |
| 402078 | 8117414 | Minus | 163935-164095, 193229-193504 |
| 403988 | 8576087 | Plus | 16251-16462 |
| 402961 | 9453790 | Plus | 42966-43193, 53444-53524 |
| 402217 | 9795981 | Minus | 21521-21757 |
| 402649 | 9958202 | Minus | 69796-70414 |
| 405046 | 7596829 | Minus | 4373-4528 |
| 403026 | 7670575 | Plus | 56521-56840 |
| 403804 | 8139999 | Minus | 21048-21443 |
| 406137 | 9166422 | Minus | 30487-31058 |
| 405146 | 9438278 | Minus | 102529-102633 |
| 403384 | 9438321 | Minus | 178616-180277 |
| 405968 | 8247789 | Plus | 14893-15148 |
| 405467 | 7770659 | Minus | 17983-18674 |
| 404321 | 9665209 | Minus | 76594-77805 |
| 403882 | 7710258 | Plus | 60805-60997, 62455-62559 |
| 402990 | 7631040 | Minus | 117579-118409 |
| 405501 | 9211311 | Minus | 49085-49400, 49565-49679, |
| | | | 50117-50262 |
| 402586 | 9908948 | Plus | 11846-12041 |
| 401612 | 7705041 | Minus | 100597-100830 |
| 404120 | 7342152 | Plus | 135775-136000 |
| 405850 | 6164995 | Plus | 13871-14110 |
| 405210 | 7230142 | Minus | 81049-81327 |
| 405979 | 8247789 | Minus | 189378-190148 |
| 402885 | 9926751 | Plus | 71919-72049 |
| 404790 | 7230958 | Plus | 38611-38761 |
| 401281 | 9800073 | Minus | 13622-15130 |
| 402504 | 9797871 | Plus | 12366-12614 |
| 400657 | 7249004 | Plus | 160793-161343 |
| 403156 | 9838039 | Minus | 82105-82989 |
| 403481 | 9965004 | Plus | 93496-93633 |
| 405333 | 3165399 | Plus | 149905-150215 |
| 402808 | 6456148 | Minus | 114964-115136, 115461-115585, |
| | | | 115931-11604 |
| 404157 | 9886596 | Minus | 47629-49050 |
| 405101 | 8076859 | Plus | 130732-132266 |
| 402299 | 6693370 | Plus | 23367-25175 |
| 406181 | 5923650 | Plus | 16586-16855 |
| 405573 | 3820491 | Minus | 32645-33144 |
| 400530 | 6981826 | Plus | 39-541 |
| 402229 | 9965022 | Minus | 15739-15951, 16166-16779 |
| 406355 | 9256052 | Minus | 97979-98656 |
| 401807 | 7331536 | Plus | 152325-152912 |
| 403347 | 8569747 | Plus | 654-1101 |
| 406043 | 6758938 | Plus | 36609-37156 |
| 404816 | 5911819 | Minus | 81825-81947, 83523-83639, |
| | | | 86204-86326 |
| 404068 | 3168621 | Minus | 18123-18766 |
| 406216 | 7382582 | Plus | 26949-27491 |
| 405945 | 6758796 | Minus | 24735-25158 |
| 404755 | 7706327 | Minus | 53729-53846 |
| 406253 | 7417725 | Plus | 62137-62741 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 2A
About 445 genes upregulated in breast metastases to the brain relative to normal body tissues

| Pkey | ExAccn | UniGeneID | UniGene Title | R1 | SEQ ID NO(s): |
|---|---|---|---|---|---|
| 408591 | AF015224 | Hs.46452 | mammaglobin 1 | 84.90 | 281 4801 |
| 429441 | AJ224172 | Hs.204096 | lipophilin B (uteroglobin family member) | 33.41 | 2371 5407 |
| 429170 | NM_001394 | Hs.2359 | dual specificity phosphatase 4 | 30.03 | 2336 5392 |
| 407276 | AI951118 | Hs.326736 | Homo sapiens breast cancer antigen NY-BR | 26.35 | 147 |
| 400292 | AA250737 | Hs.72472 | BMP-R1B (bone morphogenetic protein rec | 20.60 | 6 |
| 419078 | M93119 | Hs.89584 | insulinoma-associated 1 | 18.98 | 1272 5036 |
| 432441 | AW292425 | Hs.163484 | intron of hepatocyte nuclear factor-3 al | 17.25 | 2668 |
| 408045 | AW138959 | Hs.245123 | ESTs | 16.13 | 227 |
| 443171 | BE281128 | Hs.9030 | TONDU | 14.65 | 3501 |
| 400291 | AA401369 | | downstream of breast cancer antigen NY-B | 12.50 | 5 |
| 404561 | | | trichorhinophalangeal syndrome I gene (T | 12.48 | |
| 452838 | U65011 | Hs.30743 | preferentially expressed antigen in mela | 12.45 | 4353 5765 |
| 432415 | T16971 | Hs.289014 | ESTs, Weakly similar to A43932 mucin 2 p | 12.13 | 2665 |
| 410102 | AW248508 | Hs.279727 | ESTs; homologue of PEM-3 [Ciona savignyi | 11.43 | 446 |
| 457465 | AW301344 | Hs.122908 | DNA replication factor | 10.58 | 4592 |
| 422656 | AI870435 | Hs.1569 | LIM homeobox protein 2 | 10.13 | 1668 |
| 425057 | AA826434 | Hs.1619 | achaete-scute complex (Drosophila) homol | 10.08 | 1915 |
| 400205 | | | NM_006265*: Homo sapiens RAD21 (S. pombe) | 9.85 | 1 4680 |
| 407178 | AA195651 | | AP-2 beta transcription factor | 9.23 | 133 |
| 450705 | U90304 | Hs.25351 | iroquois homeobox protein 2A (IRX-2A) ( | 9.15 | 4153 5734 |
| 422756 | AA441787 | Hs.119689 | glycoprotein hormones, alpha polypeptide | 8.48 | 1679 |
| 447342 | AI199268 | Hs.19322 | Homo sapiens, Similar to RIKEN cDNA 2010 | 8.45 | 3845 |
| 444783 | AK001468 | Hs.62180 | anillin (Drosophila Scraps homolog), act | 8.25 | 3628 5645 |
| 401451 | | | NM_004496*: Homo sapiens hepatocyte nucle | 8.25 | 27 4697 |
| 426283 | NM_003937 | Hs.169139 | kynureninase (L-kynurenine hydrolase) | 7.73 | 2048 5297 |
| 439926 | AW014875 | Hs.137007 | ESTs | 7.69 | 3243 |
| 448275 | BE514434 | Hs.20830 | kinesin-like 2 | 7.68 | 3955 |
| 422168 | AA586894 | Hs.112408 | S100 calcium-binding protein A7 (psorias | 7.66 | 1612 |
| 416208 | AW291168 | Hs.41295 | ESTs, Weakly similar to MUC2_HUMAN MUCIN | 7.65 | 981 |
| 457211 | AW972565 | Hs.32399 | ESTs, Weakly similar to S51797 vasodilat | 7.64 | 4583 |
| 447475 | AI380797 | Hs.158992 | ESTs | 7.48 | 3865 |
| 439979 | AW600291 | Hs.6823 | hypothetical protein FLJ10430 | 7.47 | 3251 |
| 411096 | U80034 | Hs.68583 | mitochondrial intermediate peptidase | 7.43 | 4862 535 |
| 418836 | AI655499 | Hs.161712 | BMP-R1B | 7.39 | 1247 |
| 458098 | BE550224 | | metallothionein 1E (functional) | 7.23 | 4611 |
| 409731 | AA125985 | Hs.56145 | thymosin, beta, identified in neuroblast | 7.20 | 402 |
| 430287 | AW182459 | Hs.125759 | ESTs, Weakly similar to LEU5_HUMAN LEUKE | 7.05 | 2460 |
| 417576 | AA339449 | Hs.82285 | phosphoribosylglycinamide formyltransfer | 7.05 | 1102 |
| 458814 | AI498957 | Hs.170861 | ESTs, Weakly similar to Z195_HUMAN ZINC | 6.98 | 4638 |
| 451807 | W52854 | | hypothetical protein FLJ23293 similar to | 6.98 | 4249 |
| 425048 | H05468 | Hs.164502 | ESTs | 6.95 | 1914 |
| 428342 | AI739168 | | Homo sapiens cDNA FLJ13458 fis, clone PL | 6.85 | 2244 |
| 407300 | AA102616 | Hs.120769 | gb: zn43e07.s1 Stratagene HeLa cell s3 93 | 6.85 | 149 |
| 409557 | BE182896 | Hs.3686 | ESTs | 6.78 | 384 |
| 428227 | AA321649 | Hs.2248 | small inducible cytokine subfamily B (Cy | 6.75 | 2231 |
| 415786 | AW419196 | Hs.257924 | hypothetical protein FLJ13782 | 6.70 | 951 |
| 408771 | AW732573 | Hs.47584 | potassium voltage-gated channel, delayed | 6.68 | 305 |
| 445413 | AA151342 | Hs.12677 | CGI-147 protein | 6.63 | 3675 |
| 427365 | AI873274 | | downstream of breast cancer antigen NY-B | 6.62 | 2148 |
| 408908 | BE296227 | Hs.250822 | serine/threonine kinase 15 | 6.58 | 317 |
| 407999 | AI126271 | Hs.49433 | ESTs, Weakly similar to YZ28_HUMAN HYPOT | 6.47 | 222 |
| 415539 | AI733881 | Hs.72472 | BMP-R1B (bone morphogenetic protein rec | 6.47 | 935 |
| 441377 | BE218239 | Hs.202656 | ESTs | 6.45 | 3349 |
| 439963 | AW247529 | Hs.6793 | platelet-activating factor acetylhydrola | 6.44 | 3250 |
| 423242 | AL039402 | Hs.125783 | DEME-6 protein | 6.38 | 1730 |
| 427528 | AU077143 | Hs.179565 | minichromosome maintenance deficient (S. | 6.38 | 2161 |
| 452786 | R61362 | Hs.106642 | ESTs, Weakly similar to T09052 hypotheti | 6.35 | 4344 |
| 453884 | AA355925 | Hs.36232 | KIAA0186 gene product | 6.33 | 4444 |
| 413597 | AW302885 | Hs.117183 | ESTs | 6.23 | 739 |
| 418819 | AA228776 | Hs.191721 | ESTs | 6.14 | 1244 |
| 411598 | BE336654 | Hs.70937 | H3 histone family, member A | 6.08 | 562 |
| 443715 | AI583187 | Hs.9700 | cyclin E1 | 6.05 | 3544 |
| 416135 | AW473656 | Hs.227277 | ESTs | 6.03 | 975 |
| 433675 | AW977653 | Hs.75319 | ribonucleotide reductase M2 polypeptide | 5.98 | 2779 |
| 425234 | AW152225 | Hs.165909 | ESTs, Weakly similar to I38022 hypotheti | 5.84 | 1940 |
| 416933 | BE561850 | Hs.80506 | small nuclear ribonucleoprotein polypept | 5.83 | 1040 |
| 407756 | AA116021 | Hs.38260 | ubiquitin specific protease 18 | 5.67 | 191 |
| 419875 | AA853410 | Hs.93557 | proenkephalin | 5.65 | 1365 |
| 451398 | AI793124 | Hs.144479 | ESTs | 5.64 | 4219 |
| 412140 | AA219691 | Hs.73625 | RAB6 interacting, kinesin-like (rabkines | 5.57 | 613 |
| 450149 | AW969781 | Hs.132863 | Zic family member 2 (odd-paired Drosophi | 5.53 | 4106 |
| 425843 | BE313280 | Hs.159627 | death associated protein 3 | 5.51 | 2003 |
| 422805 | AA436989 | Hs.121017 | H2A histone family, member A | 5.50 | 1683 |
| 426451 | AI908165 | Hs.169946 | GATA-binding protein 3 (T-cell receptor | 5.25 | 2066 |
| 401519 | | | C15000476*: gi\|12737279\|ref\|XP_012163.1\| | 5.25 | |
| 426878 | BE069341 | | Homo sapiens breast cancer antigen NY-BR | 5.20 | 2108 |
| 407366 | AF026942 | Hs.17518 | gb: Homo sapiens cig33 mRNA, partial sequ | 5.18 | 156 |
| 432378 | AI493046 | Hs.146133 | ESTs | 5.18 | 2662 |
| 425707 | AF115402 | Hs.11713 | E74-like factor 5 (ets domain transcript | 5.15 | 1992 5277 |
| 433023 | AW864793 | | thrombospondin 1 | 5.15 | 2725 |
| 424399 | AI905687 | | AI905687: IL-BT095-190199-019 BT095 Homo | 5.14 | 1840 |
| 409269 | AA576953 | Hs.22972 | steroid 5 alpha-reductase 2-like; H5AR g | 5.13 | 358 |
| 448105 | AW591433 | Hs.298241 | Transmembrane protease, serine 3 | 5.08 | 3943 |
| 453392 | U23752 | Hs.32964 | SRY (sex determining region Y)-box 11 | 5.05 | 4403 5776 |
| 425692 | D90041 | Hs.155956 | N-acetyltransferase 1 (arylamine N-acety | 5.03 | 1989 5276 |
| 403485 | | | C3001813*: gi\|12737279\|ref\|XP_012163.1\|k | 5.03 | |
| 452461 | N78223 | Hs.108106 | transcription factor | 5.03 | 4311 |
| 443537 | D13305 | Hs.203 | cholecystokinin B receptor | 5.00 | 3528 5629 |
| 432140 | AK000404 | Hs.272688 | hypothetical protein FLJ20397 | 4.98 | 2634 5500 |
| 422486 | BE514492 | Hs.117487 | gene near HD on 4p16.3 with homology to | 4.95 | 1648 |
| 433043 | W57554 | Hs.125019 | lymphoid nuclear protein (LAF-4) mRNA | 4.88 | 2728 |
| 408035 | NM_006242 | Hs.42215 | protein phosphatase 1, regulatory subuni | 4.85 | 226 4787 |
| 424735 | U31875 | Hs.272499 | DHRS2 Dehydrogenase/reductase (SDR famil | 4.85 | 1879 5229 |
| 424905 | NM_002497 | Hs.153704 | NIMA (never in mitosis gene a)-related k | 4.85 | 1898 5237 |
| 444342 | NM_014398 | Hs.10887 | similar to lysosome-associated membrane | 4.83 | 3591 5638 |
| 419536 | AA603305 | | gb: np12d11.s1 NCI_CGAP_Pr3 Homo sapiens | 4.80 | 1327 |
| 409435 | AI810721 | Hs.95424 | ESTs | 4.78 | 370 |
| 401464 | | | histone deacetylase 5 | 4.73 | |
| 414343 | AL036166 | Hs.75914 | coated vesicle membrane protein | 4.73 | 814 |
| 453863 | X02544 | Hs.572 | orosomucoid 1 | 4.70 | 4438 5786 |
| 410467 | AF102546 | Hs.63931 | dachshund (Drosophila) homolog | 4.70 | 482 4849 |
| 415989 | AI267700 | | ESTs | 4.70 | 963 |
| 449722 | BE280074 | Hs.23960 | cyclin B1 | 4.69 | 4079 |
| 418092 | R45154 | Hs.338439 | ESTs | 4.68 | 1158 |
| 409542 | AA503020 | Hs.36563 | hypothetical protein FLJ22418 | 4.67 | 382 |
| 444858 | AI199738 | Hs.208275 | ESTs, Weakly similar to ALUA_HUMAN !!!! | 4.65 | 3633 |
| 416111 | AA033813 | Hs.79018 | chromatin assembly factor 1, subunit A ( | 4.65 | 972 |
| 450193 | AI916071 | Hs.15607 | Homo sapiens Fanconi anemia complementat | 4.61 | 4111 |
| 409902 | AI337658 | Hs.156351 | ESTs | 4.53 | 416 |
| 422835 | BE218705 | Hs.121378 | metallothionein-like 5, testis-specific | 4.50 | 1686 |
| 433323 | AA805132 | Hs.159142 | ESTs | 4.50 | 2755 |
| 448826 | AI580252 | Hs.293246 | ESTs, Weakly similar to putative p150 [H | 4.48 | 4012 |
| 401866 | | | Target Exon | 4.48 | |
| 430044 | AA464510 | Hs.152812 | ESTs | 4.43 | 2439 |
| 453439 | AI572438 | Hs.32976 | guanine nucleotide binding protein 4 | 4.38 | 4406 |
| 453160 | AI263307 | | H2B histone family, member L | 4.38 | 4380 |
| 432886 | BE159028 | Hs.279704 | chromatin accessibility complex 1 | 4.36 | 2708 |
| 414271 | AK000275 | Hs.75871 | protein kinase C binding protein 1 | 4.35 | 807 |
| 436608 | AA628980 | Hs.192371 | down syndrome critical region protein DS | 4.34 | 2997 |
| 427712 | AI368024 | Hs.283696 | ESTs | 4.34 | 2187 |
| 424085 | NM_002914 | Hs.139226 | replication factor C (activator 1) 2 (40 | 4.33 | 1803 5198 |
| 404571 | | | NM_015902*: Homo sapiens progestin induce | 4.33 | 4724 57 |
| 429986 | AF092047 | Hs.227277 | sine oculis homeobox (Drosophila) homolo | 4.25 | 2434 5431 |
| 450325 | AI935962 | Hs.91973 | ESTs | 4.23 | 4129 |
| 442861 | AA243837 | Hs.57787 | ESTs | 4.23 | 3475 |
| 431585 | BE242803 | Hs.262823 | hypothetical protein FLJ10326 | 4.23 | 2572 |
| 426501 | AW043782 | Hs.293616 | ESTs | 4.21 | 2072 |
| 448664 | AI879317 | Hs.334691 | splicing factor 3a, subunit 1, 120 kD | 4.20 | 3994 |
| 443695 | AW204099 | | ESTs, Weakly similar to AF126780 1 retin | 4.18 | 3541 |
| 432201 | AI538613 | Hs.298241 | Transmembrane protease, serine 3 | 4.16 | 2643 |
| 407980 | AA046309 | Hs.344241 | gb: zf12f01.s1 Soares_fetal_heart_NbHH19W | 4.15 | 221 |
| 433285 | AW975944 | Hs.237396 | ESTs | 4.14 | 2753 |
| 447519 | U46258 | Hs.339665 | ESTs | 4.13 | 3873 |
| 451752 | AB032997 | | KIAA1171 protein | 4.12 | 4247 5750 |
| 426581 | AB040956 | Hs.135890 | KIAA1523 protein | 4.10 | 2080 5311 |
| 436488 | BE620909 | Hs.261023 | hypothetical protein FLJ20958 | 4.10 | 2989 |
| 437389 | AL359587 | Hs.271586 | hypothetical protein DKFZp762M115 | 4.08 | 3063 5586 |
| 418700 | AI963808 | Hs.86970 | ESTs, Moderately similar to ALU5_HUMAN A | 4.08 | 1230 |
| 442760 | BE075297 | Hs.6614 | ESTs, Weakly similar to A43932 mucin 2 p | 4.08 | 3466 |
| 427427 | AF077345 | Hs.177936 | lectin, superfamily member 1 (cartilage- | 4.07 | 2153 5338 |
| 437834 | AA769294 | | gb: nz36g03.s1 NCI_CGAP_GCB1 Homo sapiens | 4.05 | 3096 |
| 436291 | BE568452 | Hs.344037 | protein regulator of cytokinesis 1 | 4.02 | 2975 |
| 441362 | BE614410 | Hs.23044 | RAD51 (S. cerevisiae) homolog (E coli Re | 4.01 | 3347 |
| 400528 | | | NM_020975*: Homo sapiens ret proto-oncoge | 4.01 | 18 4690 |
| 414706 | AW340125 | Hs.76989 | KIAA0097 gene product | 4.01 | 865 |
| 446999 | AA151520 | | hypothetical protein MGC4485 | 4.00 | 3811 |
| 434203 | BE262677 | Hs.283558 | hypothetical protein PRO1855 | 3.99 | 2820 |
| 414670 | BE409525 | Hs.902 | neurofibromin 2 (bilateral acoustic neur | 3.98 | 860 |
| 419743 | AW408762 | Hs.5957 | Homo sapiens clone 24416 mRNA sequence | 3.97 | 1356 |
| 438321 | AA576635 | Hs.6153 | CGI-48 protein | 3.97 | 3133 |
| 448686 | AA158659 | Hs.334712 | hypothetical protein FLJ14744 | 3.95 | 3997 |
| 415263 | AA948033 | Hs.130853 | ESTs | 3.93 | 920 |
| 423175 | W27595 | Hs.347310 | hypothetical protein FLJ14627 | 3.93 | 1724 |
| 433409 | AI278802 | Hs.25661 | ESTs | 3.90 | 2761 |
| 418113 | AI272141 | Hs.83484 | SRY(sex determining regionY)-box 4 | 3.88 | 1161 |
| 427811 | M81057 | Hs.180884 | carboxypeptidase B1 (tissue) | 3.86 | 2197 5346 |
| 447334 | AA515032 | Hs.91109 | ESTs | 3.86 | 3844 |
| 415621 | AI648602 | Hs.55468 | ESTs | 3.85 | 938 |
| 432840 | AK001403 | Hs.279521 | hypothetical protein FLJ20530 | 3.84 | 2704 5516 |
| 436167 | AA705651 | Hs.25087 | ESTs | 3.83 | 2965 |
| 421037 | AI684808 | Hs.197653 | ESTs | 3.83 | 1475 |
| 423165 | AI937547 | Hs.124915 | hypothetical protein MGC2601 | 3.81 | 1722 |
| 443347 | AI052543 | Hs.133244 | melanoma-derived leucine zipper, extra-n | 3.81 | 3519 |
| 424800 | AL035588 | Hs.153203 | MyoD family inhibitor | 3.81 | 1888 5232 |
| 425529 | NM_014656 | Hs.158282 | KIAA0040 gene product | 3.77 | 1975 5270 |
| 409648 | AW451449 | Hs.57749 | ESTs | 3.75 | 391 |
| 426827 | AW067805 | Hs.172665 | methylenetetrahydrofolate dehydrogenase | 3.75 | 2104 |
| 420650 | AA455706 | Hs.44581 | heat shock protein hsp70-related protein | 3.74 | 1441 |
| 434569 | AI311295 | Hs.344478 | KIAA0196 gene product | 3.73 | 2849 |
| 428654 | NM_012091 | Hs.188661 | adenosine deaminase, tRNA-specific 1 | 3.70 | 2280 5370 |
| 407378 | AA299264 | Hs.57776 | ESTs, Moderately similar to I38022 hypot | 3.70 | 160 |
| 404632 | | | NM_022490: Homo sapiens hypothetical prot | 3.68 | 4726 59 |
| 414004 | AA737033 | Hs.7155 | similar to thymidylate kinase family LPS | 3.67 | 772 |
| 448595 | AB014544 | Hs.21572 | KIAA0644 gene product | 3.66 | 3987 5711 |
| 429922 | Z97630 | Hs.226117 | H1 histone family, member 0 | 3.66 | 2427 5430 |
| 419440 | AB020689 | Hs.90419 | KIAA0882 protein | 3.66 | 1313 5047 |
| 439453 | BE264974 | Hs.6566 | thyroid hormone receptor interactor 13 | 3.66 | 3208 |
| 456508 | AA502764 | Hs.123469 | ESTs, Weakly similar to AF208855 1 BM-01 | 3.66 | 4547 |
| 433701 | AW445023 | Hs.15155 | ESTs | 3.65 | 2782 |
| 420390 | AA330047 | Hs.191187 | ESTs | 3.65 | 1418 |
| 418661 | NM_001949 | Hs.1189 | E2F transcription factor 3 | 3.65 | 1222 5022 |
| 418203 | X54942 | Hs.83758 | CDC28 protein kinase 2 | 3.64 | 1169 5003 |
| 434844 | AF157116 | Hs.22350 | hypothetical protein LOC56757 | 3.63 | 2873 |
| 424179 | F30712 | Hs.334573 | Homo sapiens, clone IMAGE: 4285740, mRNA | 3.63 | 1812 |
| 447350 | AI375572 | | v-erb-a avian erythroblastic leukemia vi | 3.60 | 3849 |
| 444743 | AA045648 | Hs.301957 | nudix (nucleoside diphosphate linked moi | 3.60 | 3624 |
| 430839 | U67918 | Hs.248049 | fibroblast growth factor 10 | 3.60 | 2519 5464 |
| 422309 | U79745 | Hs.114924 | solute carrier family 16 (monocarboxylic | 3.58 | 1630 5146 |
| 439772 | AL365406 | Hs.10268 | Homo sapiens mRNA full length insert cDN | 3.58 | 3234 |
| 435664 | AI032087 | Hs.269819 | ESTs | 3.57 | 2936 |
| 428134 | AA421773 | Hs.161008 | ESTs | 3.55 | 2221 |
| 452092 | BE245374 | Hs.27842 | hypothetical protein FLJ11210 | 3.54 | 4268 |
| 438869 | AF075009 | | gb: Homo sapiens full length insert cDNA | 3.53 | 3171 |
| 410555 | U92649 | Hs.64311 | a disintegrin and metalloproteinase doma | 3.53 | 4851 492 |
| 452994 | AW962597 | Hs.31305 | KIAA1547 protein | 3.51 | 4363 |
| 402496 | | | Target Exon | 3.51 | |
| 429353 | AL117406 | Hs.335891 | ATP-binding cassette transporter MRP8 | 3.49 | 2359 |
| 423419 | R55336 | Hs.23539 | ESTs | 3.48 | 1742 |
| 441690 | R81733 | Hs.33106 | ESTs | 3.47 | 3369 |
| 407235 | D20569 | Hs.169407 | SAC2 (suppressor of actin mutations 2, y | 3.47 | 140 |
| 412970 | AB026436 | Hs.177534 | dual specificity phosphatase 10 | 3.45 | 4890 687 |
| 400880 | | | NM_000611*: Homo sapiens CD59 antigen p18 | 3.45 | 23 4694 |
| 409456 | U34962 | Hs.54473 | cardiac-specific homeo box | 3.44 | 374 4825 |
| 457579 | AB030816 | Hs.36761 | HRAS-like suppressor | 3.44 | 4595 5813 |
| 409430 | R21945 | Hs.346735 | splicing factor, arginine/serine-rich 5 | 3.43 | 369 |
| 406922 | S70284 | Hs.119597 | gb: stearoyl-CoA desaturase [human, adipo | 3.43 | 109 4755 |
| 418304 | AA215702 | | gb: zr97g10.r1 NCI_CGAP_GCB1 Homo sapiens | 3.43 | 1178 |
| 428479 | Y00272 | Hs.334562 | cell division cycle 2, G1 to S and G2 to | 3.43 | 2264 5363 |
| 444670 | H58373 | Hs.332938 | hypothetical protein MGC5370 | 3.43 | 3618 |
| 400277 | | | Eos Control | 3.42 | |
| 440273 | AI805392 | Hs.325335 | Homo sapiens cDNA: FLJ23523 fis, clone L | 3.39 | 3274 |
| 452833 | BE559681 | Hs.30736 | KIAA0124 protein | 3.39 | 4351 |
| 413450 | Z99716 | Hs.75372 | N-acetylgalactosaminidase, alpha- | 3.39 | 4901 723 |
| 453900 | AW003582 | Hs.226414 | ESTs, Weakly similar to ALU8_HUMAN ALU S | 3.38 | 4448 |
| 415402 | AA164687 | Hs.177576 | mannosyl (alpha-1,3-)-glycoprotein beta- | 3.37 | 930 |
| 426931 | NM_003416 | Hs.2076 | zinc finger protein 7 (KOX 4, clone HF.1 | 3.37 | 2114 5328 |
| 426384 | AI472078 | Hs.303662 | hypothetical protein FLJ13189 (FLJ13189) | 3.35 | 2060 |
| 425782 | U66468 | Hs.159525 | cell growth regulatory with EF-hand doma | 3.35 | 1996 5278 |
| 420005 | AW271106 | Hs.133294 | ESTs | 3.35 | 1372 |
| 425548 | AA890023 | Hs.1906 | prolactin receptor | 3.35 | 1978 |
| 441790 | AW294909 | Hs.132208 | ESTs | 3.35 | 3372 |
| 429084 | AJ001443 | Hs.195614 | splicing factor 3b, subunit 3, 130 kD | 3.34 | 2332 5390 |
| 446258 | AI283476 | Hs.263478 | ESTs | 3.34 | 3740 |
| 420090 | AA220238 | Hs.94986 | ribonuclease P (38 kD) | 3.33 | 1383 |
| 439352 | BE614347 | Hs.169615 | hypothetical protein FLJ20989 | 3.31 | 3202 |
| 411558 | AA102670 | Hs.70725 | gamma-aminobutyric acid (GABA) A recepto | 3.30 | 560 |
| 444371 | BE540274 | Hs.239 | forkhead box M1 | 3.30 | 3592 |
| 437967 | BE277414 | Hs.5947 | met transforming oncogene (derived from | 3.29 | 3112 |
| 421305 | BE397354 | Hs.324830 | diptheria toxin resistance protein requi | 3.29 | 1505 |
| 406685 | AA18728 | | gb: Human nonspecific crossreacting antig | 3.29 | 4745 83 |
| 418478 | U38945 | Hs.1174 | cyclin-dependent kinase inhibitor 2A (me | 3.28 | 1204 5017 |
| 401558 | | | ENSP00000220478*: SECRETOGRANIN III. | 3.28 | |
| 407021 | U52077 | | gb: Human mariner1 transposase gene, comp | 3.27 | 119 4762 |
| 446054 | AB014537 | Hs.13604 | KIAA0637 gene product | 3.27 | 3722 5664 |
| 441020 | W79283 | Hs.35962 | ESTs | 3.26 | 3325 |
| 441128 | AA570256 | | ESTs, Weakly similar to T23273 hypotheti | 3.24 | 3334 |
| 447349 | AI375546 | | gb: tc23d04.x1 Soares_total_fetus_Nb2HF8_{—} | 3.24 | 3848 |
| 434378 | AA631739 | Hs.335440 | EST | 3.24 | 2836 |
| 400295 | W72838 | | AI905687: IL-BT095-190199-019 BT095 Homo | 3.24 | 8 |
| 452206 | AW340281 | Hs.33074 | Homo sapiens, clone IMAGE: 3606519, mRNA, | 3.23 | 4281 |
| 443162 | T49951 | Hs.9029 | DKFZP434G032 protein | 3.22 | 3500 |
| 427658 | H61387 | Hs.30868 | nogo receptor | 3.21 | 2175 |
| 458621 | AI221741 | Hs.117777 | ESTs | 3.21 | 4630 |
| 422938 | NM_001809 | Hs.1594 | centromere protein A (17 kD) | 3.20 | 1694 5162 |
| 424871 | NM_004525 | Hs.153595 | low density lipoprotein-related protein | 3.20 | 1892 5234 |
| 410340 | AW182833 | Hs.112188 | hypothetical protein FLJ13149 | 3.20 | 466 |
| 416294 | D86980 | Hs.79170 | KIAA0227 protein | 3.20 | 4958 984 |
| 417386 | AL037228 | Hs.82043 | D123 gene product | 3.20 | 1090 |
| 418004 | U37519 | Hs.87539 | aldehyde dehydrogenase 3 family, member | 3.20 | 1145 4997 |
| 444461 | R53734 | Hs.25978 | ESTs, Weakly similar to 2109260A B cell | 3.18 | 3600 |
| 410174 | AA306007 | Hs.59461 | DKFZP434C245 protein | 3.17 | 453 |
| 408393 | AW015318 | Hs.23165 | ESTs | 3.17 | 263 |
| 426215 | AW963419 | Hs.155223 | stanniocalcin 2 | 3.17 | 2039 |
| 417771 | AA804698 | Hs.82547 | retinoic acid receptor responder (tazaro | 3.16 | 1121 |
| 453005 | AW055308 | Hs.31803 | ESTs, Weakly similar to N-WASP [H. sapien | 3.15 | 4365 |
| 413278 | BE563085 | Hs.833 | interferon-stimulated protein, 15 kDa | 3.15 | 705 |
| 441134 | W29092 | Hs.346950 | cellular retinoic acid-binding protein 1 | 3.14 | 3335 |
| 420802 | U22376 | Hs.1334 | v-myb avian myeloblastosis viral oncogen | 3.13 | 1455 5087 |
| 449746 | AI668594 | Hs.176588 | ESTs, Weakly similar to CP4Y_HUMAN CYTOC | 3.13 | 4080 |
| 417601 | NM_014735 | Hs.82292 | KIAA0215 gene product | 3.12 | 1105 4991 |
| 421654 | AW163267 | Hs.106469 | suppressor of var1 (S. cerevisiae) 3-like | 3.11 | 1550 |
| 408877 | AA479033 | Hs.130315 | ESTs, Weakly similar to A47582 B-cell gr | 3.10 | 314 |
| 453511 | AL031224 | Hs.33102 | AP-2 beta transcription factor | 3.10 | 4416 5779 |
| 422981 | AF026445 | Hs.122752 | TATA box binding protein (TBP)-associate | 3.10 | 1706 5165 |
| 413374 | NM_001034 | Hs.75319 | ribonucleotide reductase M2 polypeptide | 3.10 | 4899 713 |
| 449704 | AK000733 | Hs.23900 | GTPase activating protein | 3.10 | 4076 5722 |
| 427581 | NM_014788 | Hs.179703 | KIAA0129 gene product | 3.08 | 2167 5339 |
| 418329 | AW247430 | Hs.84152 | cystathionine-beta-synthase | 3.05 | 1186 |
| 422010 | AA302049 | Hs.31181 | Homo sapiens cDNA: FLJ23230 fis, clone C | 3.04 | 1593 |
| 409892 | AW956113 | Hs.7149 | gb: EST368183 MAGE resequences, MAGD Homo | 3.04 | 414 |
| 427674 | NM_003528 | Hs.2178 | H2B histone family, member Q | 3.04 | 2177 5342 |
| 431745 | AW972448 | Hs.163425 | Novel FGENESH predicted cadherin repeat | 3.03 | 2595 |
| 409757 | NM_001898 | Hs.123114 | cystatin SN | 3.03 | 403 4832 |
| 417288 | AI984792 | Hs.108812 | hypothetical protein FLJ22004 | 3.03 | 1077 |
| 420111 | AA255652 | | gb: zs21h11.r1 NCI_CGAP_GCB1 Homo sapiens | 3.03 | 1386 |
| 428771 | AB028992 | Hs.193143 | KIAA1069 protein | 3.03 | 2295 5375 |
| 408527 | AL135018 | Hs.33074 | Homo sapiens, clone IMAGE: 3606519, mRNA, | 3.02 | 276 |
| 424001 | W67883 | Hs.137476 | paternally expressed 10 | 3.02 | 1788 |
| 420552 | AK000492 | Hs.98806 | hypothetical protein | 3.02 | 1430 5081 |
| 413063 | AL035737 | Hs.75184 | chitinase 3-like 1 (cartilage glycoprote | 3.01 | 692 |
| 410619 | BE512730 | Hs.65114 | keratin 18 | 3.01 | 498 |
| 453902 | BE502341 | Hs.3402 | ESTs | 3.01 | 4449 |
| 423645 | AI215632 | Hs.147487 | ESTs | 3.00 | 1764 |
| 442353 | BE379594 | Hs.49136 | ESTs, Moderately similar to ALU7_HUMAN A | 3.00 | 3423 |
| 425842 | AI587490 | Hs.159623 | NK-2 (Drosophila) homolog B | 3.00 | 2002 |
| 439680 | AW245741 | Hs.58461 | ESTs, Weakly similar to A35659 krueppel- | 2.99 | 3229 |
| 442530 | AI580830 | Hs.176508 | Homo sapiens cDNA FLJ14712 fis, clone NT | 2.99 | 3437 |
| 430066 | AI929659 | Hs.237825 | signal recognition particle 72 kD | 2.99 | 2442 |
| 420649 | AI866964 | Hs.124704 | ESTs, Moderately similar to S65657 alpha | 2.98 | 1440 |
| 437915 | AI637993 | Hs.202312 | Homo sapiens clone N11 NTera2D1 teratoca | 2.98 | 3105 |
| 452256 | AK000933 | Hs.28661 | Homo sapiens cDNA FLJ10071 fis, clone HE | 2.98 | 4289 |
| 433029 | NM_014322 | Hs.279926 | opsin 3 (encephalopsin) | 2.97 | 2726 5524 |
| 417924 | AU077231 | Hs.82932 | cyclin D1 (PRAD1: parathyroid adenomatos | 2.97 | 1138 |
| 455286 | BE144384 | | gb: MR0-HT0166-191199-004-c11 HT0166 Homo | 2.95 | 4504 |
| 437682 | AA476652 | Hs.94952 | Homo sapiens cDNA: FLJ23371 fis, clone H | 2.95 | 3083 |
| 408633 | AW963372 | Hs.46677 | PRO2000 protein | 2.95 | 286 |
| 401203 | | | Target Exon | 2.95 | |
| 418675 | AW299723 | Hs.87223 | bone morphogenetic protein receptor, typ | 2.95 | 1225 |
| 428166 | AA423849 | Hs.79530 | M5-14 protein | 2.95 | 2224 |
| 440594 | AW445167 | Hs.126036 | ESTs | 2.95 | 3302 |
| 430294 | AI538226 | Hs.32976 | guanine nucleotide binding protein 4 | 2.94 | 2463 |
| 434094 | AA305599 | Hs.238205 | hypothetical protein PRO2013 | 2.94 | 2814 |
| 412141 | AI183838 | Hs.48938 | hypothetical protein FLJ21802 | 2.94 | 614 |
| 430289 | AK001952 | Hs.238039 | hypothetical protein FLJ11090 | 2.94 | 2461 5440 |
| 441021 | AW578716 | Hs.7644 | H1 histone family, member 2 | 2.93 | 3326 |
| 439580 | AF086401 | Hs.293847 | ESTs, Moderately similar to S65657 alpha | 2.93 | 3220 |
| 423198 | M81933 | Hs.1634 | cell division cycle 25A | 2.92 | 1727 5174 |
| 453751 | R36762 | Hs.101282 | Homo sapiens cDNA: FLJ21238 fis, clone C | 2.92 | 4428 |
| 412942 | AL120344 | Hs.75074 | mitogen-activated protein kinase-activat | 2.90 | 685 |
| 424036 | AA770688 | | H2A histone family, member L | 2.90 | 1793 |
| 442711 | AF151073 | Hs.8645 | hypothetical protein | 2.90 | 3460 5625 |
| 427719 | AI393122 | Hs.134726 | ESTs | 2.89 | 2189 |
| 443845 | AI590084 | | ESTs, Weakly similar to A47161 Mac-2-bin | 2.89 | 3560 |
| 432731 | R31178 | Hs.287820 | fibronectin 1 | 2.89 | 2695 |
| 444079 | H09048 | Hs.23606 | ESTs | 2.88 | 3572 |
| 432728 | NM_006979 | Hs.278721 | HLA class II region expressed gene KE4 | 2.88 | 2694 5512 |
| 422955 | AW967824 | Hs.324237 | ESTs | 2.88 | 1697 |
| 432116 | AA902953 | Hs.308538 | ESTs | 2.88 | 2632 |
| 427521 | AW973352 | | ESTs | 2.88 | 2159 |
| 431127 | U66618 | Hs.250581 | SWI/SNF related, matrix associated, acti | 2.87 | 2532 |
| 416636 | N32536 | Hs.42645 | solute carrier family 16 (monocarboxylic | 2.87 | 1008 |
| 409703 | NM_006187 | Hs.56009 | 2′-5′-oligoadenylate synthetase 3 (100 k | 2.87 | 396 4831 |
| 440074 | AA863045 | Hs.10669 | ESTs, Weakly similar to T00050 hypotheti | 2.87 | 3259 |
| 411285 | AI733766 | Hs.69429 | Homo sapiens IMAGE: 512024 clone, mRNA | 2.86 | 545 |
| 442772 | AW503680 | Hs.5957 | Homo sapiens clone 24416 mRNA sequence | 2.86 | 3468 |
| 431566 | AF176012 | Hs.260720 | J domain containing protein 1 | 2.85 | 2568 5479 |
| 405366 | | | NM_003371*: Homo sapiens vav 2 oncogene ( | 2.85 | 4735 69 |
| 414747 | U30872 | Hs.77204 | centromere protein F (350/400 kD, mitosin | 2.85 | 4927 873 |
| 452099 | BE612992 | Hs.27931 | hypothetical protein FLJ10607 similar to | 2.83 | 4270 |
| 405770 | | | NM_002362: Homo sapiens melanoma antigen, | 2.83 | 4740 74 |
| 439024 | R96696 | Hs.35598 | ESTs | 2.83 | 3183 |
| 420440 | NM_002407 | Hs.97644 | mammaglobin 2 | 2.82 | 1422 5076 |
| 424339 | BE257148 | | endoglycan | 2.82 | 1831 |
| 439574 | AI469788 | | ESTs | 2.82 | 3219 |
| 438714 | AA814859 | | ESTs | 2.81 | 3161 |
| 448789 | BE539108 | Hs.22051 | hypothetical protein MGC15548 | 2.80 | 4007 |
| 431546 | L39211 | Hs.259785 | carnitine palmitoyltransferase I, liver | 2.80 | 2563 5478 |
| 428898 | AB033070 | Hs.194408 | KIAA1244 protein | 2.80 | 2316 5383 |
| 452826 | BE245286 | Hs.301636 | peroxisomal biogenesis factor 6 | 2.78 | 4349 |
| 406277 | | | Target Exon | 2.78 | |
| 423554 | M90516 | Hs.1674 | glutamine-fructose-6-phosphate transamin | 2.78 | 1758 5182 |
| 437108 | AA434054 | Hs.80624 | hypothetical protein MGC2560 | 2.77 | 3034 |
| 410275 | U85658 | Hs.61796 | transcription factor AP-2 gamma (activat | 2.77 | 462 4842 |
| 405558 | | | Target Exon | 2.77 | |
| 452620 | AA436504 | Hs.119286 | ESTs | 2.77 | 4330 |
| 406625 | Y13647 | Hs.119597 | stearoyl-CoA desaturase (delta-9-desatur | 2.77 | 4744 79 |
| 416128 | AA173632 | | CDC14 (cell division cycle 14, S. cerevi | 2.76 | 974 |
| 448877 | AI583696 | Hs.253313 | ESTs | 2.76 | 4016 |
| 431882 | NM_001426 | Hs.271977 | engrailed homolog 1 | 2.75 | 2612 5493 |
| 411678 | AI907114 | Hs.71465 | squalene epoxidase | 2.75 | 568 |
| 449318 | AW236021 | Hs.78531 | Homo sapiens, Similar to RIKEN cDNA 5730 | 2.75 | 4055 |
| 433388 | AI432672 | Hs.288539 | hypothetical protein FLJ22191 | 2.75 | 2759 |
| 409178 | BE393948 | Hs.50915 | kallikrein 5 | 2.75 | 345 |
| 409960 | BE261944 | | hexokinase 1 | 2.74 | 422 |
| 408296 | AL117452 | Hs.44155 | DKFZP586G1517 protein | 2.74 | 252 4792 |
| 422559 | AW247696 | Hs.155839 | hypothetical protein MGC12934 | 2.73 | 1656 |
| 415889 | R24563 | | VPS10 domain receptor protein | 2.73 | 957 |
| 406043 | | | Target Exon | 2.73 | |
| 440351 | AF030933 | Hs.7179 | RAD1 (S. pombe) homolog | 2.73 | 3285 5607 |
| 411630 | U42349 | Hs.71119 | Putative prostate cancer tumor suppresso | 2.73 | 4866 565 |
| 421077 | AK000061 | Hs.101590 | hypothetical protein | 2.72 | 1479 5093 |
| 407204 | R41933 | Hs.140237 | ESTs, Weakly similar to ALU1_HUMAN ALU S | 2.72 | 136 |
| 435532 | AW291488 | Hs.117305 | Homo sapiens, clone IMAGE: 3682908, mRNA | 2.70 | 2923 |
| 446163 | AA026880 | Hs.25252 | prolactin receptor | 2.70 | 3731 |
| 412482 | AI499930 | Hs.334885 | mitochondrial GTP binding protein | 2.70 | 636 |
| 409235 | AA188827 | Hs.7988 | ESTs, Weakly similar to I38022 hypotheti | 2.70 | 354 |
| 407242 | M18728 | | gb: Human nonspecific crossreacting antig | 2.69 | 142 4766 |
| 449349 | AI825386 | | hypothetical protein FLJ21939 similar to | 2.68 | 4057 |
| 448552 | AW973653 | Hs.20104 | hypothetical protein FLJ00052 | 2.68 | 3983 |
| 404580 | | | trichorhinophalangeal syndrome I gene (T | 2.68 | |
| 416198 | H27332 | Hs.99598 | hypothetical protein MGC5338 | 2.68 | 980 |
| 442643 | U82756 | | PRP4/STK/WD splicing factor | 2.67 | 3457 5623 |
| 422363 | T55979 | Hs.115474 | replication factor C (activator 1) 3 (38 | 2.67 | 1636 |
| 407137 | T97307 | | gb: ye53h05.s1 Soares fetal liver spleen | 2.67 | 128 |
| 404076 | | | NM_016020*: Homo sapiens CGI-75 protein ( | 2.67 | 4719 51 |
| 447805 | AW627932 | Hs.302421 | gemin4 | 2.67 | 3908 |
| 450256 | AA286887 | Hs.24724 | MFH-amplified sequences with leucine-ric | 2.66 | 4120 |
| 452834 | AI638627 | Hs.105685 | KIAA1688 protein | 2.66 | 4352 |
| 439949 | AW979197 | Hs.292073 | ESTs, Weakly similar to ALU7_HUMAN ALU S | 2.66 | 3248 |
| 416677 | T83470 | Hs.334840 | ESTs, Moderately similar to I78885 serin | 2.65 | 1012 |
| 434540 | NM_016045 | Hs.3945 | CGI-107 protein | 2.65 | 2847 5549 |
| 404857 | | | ENSP00000215851*: DJ930L11.1 (SIMILAR TO | 2.65 | |
| 450728 | AW162923 | Hs.25363 | presenilin 2 (Alzheimer disease 4) | 2.65 | 4156 |
| 430452 | AI888450 | Hs.174644 | hypothetical protein FLJ21669 | 2.65 | 2486 |
| 425018 | BE245277 | Hs.154196 | E4F transcription factor 1 | 2.65 | 1912 |
| 419767 | W73306 | Hs.306668 | Homo sapiens cDNA FLJ14089 fis, clone MA | 2.65 | 1361 |
| 435124 | AA725362 | Hs.120456 | ESTs | 2.65 | 2897 |
| 426919 | AL041228 | | ELAV (embryonic lethal, abnormal vision, | 2.65 | 2111 |
| 407168 | R45175 | Hs.117183 | ESTs | 2.65 | 131 |
| 422880 | AF228704 | Hs.193974 | glutathione reductase | 2.63 | 1689 5161 |
| 409799 | D11928 | Hs.76845 | phosphoserine phosphatase-like | 2.63 | 407 |
| 402102 | | | Target Exon | 2.63 | |
| 452243 | AL355715 | Hs.28555 | programmed cell death 9 (PDCD9) | 2.63 | 4287 5756 |
| 429901 | AK000502 | Hs.56237 | hypothetical protein FLJ20495 | 2.62 | 2424 5429 |
| 454425 | AW300927 | Hs.27192 | hypothetical protein dJ1057B20.2 | 2.62 | 4482 |
| 407792 | AI077715 | Hs.39384 | putative secreted ligand homologous to f | 2.61 | 196 |
| 407777 | AA161071 | Hs.71465 | squalene epoxidase | 2.61 | 194 |
| 451369 | AA017321 | Hs.269691 | ESTs | 2.61 | 4216 |
| 432586 | AA568548 | | ESTs | 2.60 | 2681 |
| 415632 | U67085 | Hs.78524 | TcD37 homolog | 2.60 | 4950 939 |
| 419526 | AI821895 | Hs.193481 | ESTs | 2.60 | 1325 |
| 400884 | | | Target Exon | 2.60 | |
| 425671 | AF193612 | Hs.159142 | lunatic fringe (Drosophila) homolog | 2.60 | 1984 5274 |
| 425236 | AW067800 | Hs.155223 | stanniocalcin 2 | 2.60 | 1941 |
| 453507 | AF083217 | Hs.33085 | WD repeat domain 3 | 2.59 | 4414 5778 |
| 423081 | AF262992 | Hs.123159 | sperm associated antigen 4 | 2.58 | 1717 5167 |
| 422771 | NM_012318 | Hs.120165 | leucine zipper-EF-hand containing transm | 2.58 | 1681 5158 |
| 419589 | AW973708 | Hs.201925 | Homo sapiens cDNA FLJ13446 fis, clone PL | 2.58 | 1336 |
| 403006 | | | NM_006933*: Homo sapiens solute carrier f | 2.58 | 41 4710 |
| 453785 | AI368236 | Hs.283732 | ESTs, Moderately similar to ALU1_HUMAN A | 2.58 | 4432 |
| 418437 | AA771738 | Hs.348000 | ESTs, Moderately similar to ALU5_HUMAN A | 2.58 | 1201 |
| 436211 | AK001581 | Hs.334828 | hypothetical protein FLJ10719; KIAA1794 | 2.58 | 2967 5576 |
| 406627 | T64904 | Hs.163780 | ESTs | 2.57 | 80 |
| 431130 | NM_006103 | Hs.2719 | HE4; epididymis-specific, whey-acidic pr | 2.57 | 2533 5470 |
| 424308 | AW975531 | Hs.154443 | minichromosome maintenance deficient (S. | 2.57 | 1827 |
| 404982 | | | Target Exon | 2.56 | |
| 453968 | AA847843 | Hs.62711 | High mobility group (nonhistone chromoso | 2.56 | 4456 |
| 412673 | AL042957 | Hs.31845 | ESTs | 2.56 | 659 |
| 433848 | AF095719 | Hs.93764 | carboxypeptidase A4 | 2.56 | 2790 5541 |
| 434815 | AF155582 | Hs.46744 | core1 UDP-galactose: N-acetylgalactosamin | 2.55 | 2870 5555 |
| 454110 | AA195509 | Hs.39733 | postsynaptic protein CRIPT | 2.55 | 4469 |
| 418617 | AA225849 | Hs.83419 | ESTs, Moderately similar to ALU8_HUMAN A | 2.55 | 1217 |
| 410507 | AA355288 | | transitional epithelia response protein | 2.55 | 486 |
| 442326 | H92962 | Hs.124813 | hypothetical protein MGC14817 | 2.55 | 3417 |
| 454453 | AW752781 | | hypothetical protein FLJ12614 similar to | 2.54 | 4485 |
| 430316 | NM_000875 | Hs.239176 | insulin-like growth factor 1 receptor | 2.54 | 2470 5445 |
| 442961 | BE614474 | | F-box only protein 22 | 2.53 | 3484 |
| 428484 | AF104032 | Hs.184601 | solute carrier family 7 (cationic amino | 2.53 | 2265 5364 |
| 430375 | AW371048 | Hs.93758 | H4 histone family, member H | 2.53 | 2477 |
| 423250 | BE061916 | Hs.125849 | chromosome 8 open reading frame 2 | 2.53 | 1732 |
| 440006 | AK000517 | Hs.6844 | NALP2 protein; PYRIN-Containing APAF1-li | 2.53 | 3252 5601 |
| 426098 | NM_014906 | Hs.166351 | KIAA1072 protein | 2.53 | 2026 5291 |
| 415857 | AA866115 | Hs.127797 | Homo sapiens cDNA FLJ11381 fis, clone HE | 2.53 | 956 |
| 426108 | AA622037 | Hs.166468 | programmed cell death 5 | 2.52 | 2028 |
| 425159 | NM_004341 | Hs.154868 | carbamoyl-phosphate synthetase 2, aspart | 2.52 | 1931 5249 |
| 422390 | AW450893 | Hs.121830 | ESTs, Weakly similar to T42682 hypotheti | 2.52 | 1638 |
| 411605 | AW006831 | | ESTs | 2.51 | 563 |
| 421690 | AW162667 | Hs.106857 | calbindin 2, (29 kD, calretinin) | 2.51 | 1554 |
| 452827 | AI571835 | Hs.55468 | ESTs | 2.50 | 4350 |
| 431958 | X63629 | Hs.2877 | cadherin 3, type 1, P-cadherin (placenta | 2.50 | 2621 5498 |
| 459376 | BE258770 | | Homo sapiens, clone IMAGE: 3344506, mRNA, | 2.50 | 4667 |
| 452335 | AW188944 | Hs.61272 | ESTs | 2.50 | 4297 |
| 447397 | BE247676 | Hs.18442 | E-1 enzyme | 2.50 | 3856 |
| 426991 | AK001536 | Hs.214410 | Homo sapiens cDNA FLJ10674 fis, clone NT | 2.50 | 2117 |
| 414915 | NM_002462 | Hs.76391 | myxovirus (influenza) resistance 1, homo | 2.50 | 4934 892 |

Pkey: Unique Eos probeset identifier number
ExAccn: Exemplar Accession number, Genbank accession number
UniGeneID: UniGene number
UniGene Title: UniGene gene title
R1: 90th percentile of breast metastases to the brain Als divided by the 90th percentile of normal body tissue Als, where the 15th percentile of all normal body tissue Als was subtracted from the numerator and denominator.
SEQ ID NO(s): SEQ ID number(s) for nucleic acid and protein sequences associated with table entry.

TABLE 2B

| Pkey | CAT number | Accession |
|---|---|---|
| 400291 | 1314911_1 | AA927862 AA401369 AI873274 |
| 400205 | 2538_1 | NM_006265 D38551 X98294 BM477931 BM461566 AU123557 AU133303 AU134649 AW500421 BM172439 AW500587 AW503665 |
| | | AW504355 AW503640 BM152454 AW505260 AI815984 AW504075 AW500716 AL597310 BC001229 BM474371 AA984202 AU135205 |
| | | BE090841 AW163750 BF747730 BF898637 AI206506 AV660870 AV692110 AW386830 AV656831 N84710 AW993470 BF086802 |
| | | BF758454 BG960772 BF757769 BI870853 BE018627 C75436 AW148744 BF757753 BG622067 BE909924 AA708208 BG530266 BF968015 |
| | | AW992930 BF888862 BG536628 AA143164 AW748953 BG498922 BF885190 BF889005 BF754781 BF800003 BM476529 AI627668 |
| | | AW028126 AL046011 BF590668 AI017447 AA579936 AI367597 AA699622 BE280597 AI124620 AI082548 AW274985 AA677870 AI056767 |
| | | BE551689 AA287642 H94499 AI752427 AI652365 AW002374 AW062651 AA360834 N68822 AU135442 AU125960 Z78334 BE545813 |
| | | AI092115 BF312771 BF242859 BG533616 BG533761 BG164745 BG492433 BM473183 AA172043 AA172069 AU157092 AU151353 |
| | | AU155318 BE302211 AI375022 AA085641 AU157923 H88858 AA132730 AA115113 AA909781 AI475256 AA424206 AW572383 AW084296 |
| | | AI184820 AI469178 AA782432 H92184 AA340562 BF195818 AA852821 AW576342 AA827107 AA173317 AW190014 AI918514 AA729372 |
| | | AA729718 AI055958 AA331424 BE328601 AA515690 BI018896 AW628277 AA748368 AA626222 BG492636 AW380620 BF800058 |
| | | AW370956 AA290909 R25857 BG952995 BF801437 AA172077 AU155890 AU149783 AI720904 AA902936 AA865727 AI470830 AV740677 |
| | | AA142982 AA482485 AU145485 AW576399 AU156042 R63448 BF246427 BE928472 D25910 BF758439 BF968785 BE565238 AA355981 |
| | | AI905607 BG291148 BG533096 BG532888 BF030886 BG613756 BE928471 BG574501 AA187596 AA361196 T95557 BG531446 |
| | | BG527242 BG527513 BG611106 AA085995 BF847252 BG024608 BE540261 BG531236 AL579993 BG108733 BG483503 BG571032 |
| | | BG492505 |
| 407178 | 683007_1 | AW235123 AA195651 |
| 458098 | 23945_1 | AI082245 BE467534 AI797130 BE467063 BE467767 BE218421 AI694996 BE327781 BE327407 BE833829 AA989054 AA459718 BE833855 |
| | | BE550224 AA832519 AF086393 AV733386 BE465409 N29245 W07677 AA482971 BE503548 H18151 AA461301 W79223 W74510 |
| | | AI090689 AL600773 AL600781 N46003 R28075 R34182 BE071550 AW885857 AI276145 AI276696 H97808 N20540 AI468553 |
| 451807 | 17758_2 | BM479185 AL552795 AL577722 BF038888 BM127617 BF510346 AW450652 AA865478 AW449519 BM127314 AI806539 AW449522 |
| | | AA993634 AI827626 AA904788 |
| 428342 | 6712_1 | AK056315 AI015524 AA724079 BI713619 AI377728 AW293682 AI928140 AI092404 AI085630 AA731340 BM469629 AW968804 AA425658 |
| | | AA769094 BF446026 AW118719 AI332765 AW500888 AW576556 AI859571 AW499664 AW614573 AW629495 AW505314 W74704 |
| | | AI356361 AI923640 AW070509 AI521500 AL042095 AA609309 AA761319 AI381489 H45700 AA761333 AW265424 AA909524 AA635311 |
| | | AA649040 AI392620 Z40708 AI985564 AW263513 AA913892 AI693486 AW263502 AI806164 AW291137 BI061872 BI059498 AA134476 AW084888 AA036967 AW370823 T55263 |
| | | BI002756 AA489664 BF827261 W74741 BF963166 |
| 427365 | 1314911_1 | AA927862 AA401369 AI873274 |
| 426878 | 1026976_1 | AL044891 AI908240 AA393080 AW748403 BE069341 BF330573 |
| 433023 | 3970_8 | BE999967 BF438599 AW864793 AI802899 BE815132 AW468888 AI672189 AI052004 BF112024 AA772335 AW275054 AA573845 |
| | | AI144148 AI968683 AA846676 AA927355 H80424 AW973295 R88209 F29868 BE928871 |
| 424399 | 2196_1 | NM_058173 AF414087 W72837 BF742809 AW070916 BE092421 AI905687 AA340069 BE074512 AI905623 AI905633 BG202312 W72838 |
| | | AI139456 BG218084 BE926938 BE186013 AW176044 AW291950 BG185269 BG197186 BG192597 BG183176 BG207535 AI127172 |
| | | BE815819 AI905624 R75793 BG202313 AI905837 BE815853 |
| 419536 | 251846_1 | AA244095 AA603305 AA244183 |
| 415989 | 10194_1 | BC013389 BC017398 AI023543 AA191424 AI267700 AI469633 AW958465 AW953397 AA172056 BE940298 BF909208 BF909980 |
| | | BF095153 BG285837 AI720344 BF541715 AA355086 AA172236 |
| 453160 | 6028_5 | BC009612 NM_003526 BI597616 AV761592 AV760377 AL601008 BI604131 BE645918 BG187760 BG181525 BG210634 BG192999 |
| | | AI263307 AA344186 AW952966 AA033609 AA037562 AA722183 R79452 H70775 BF674991 BE769437 BG007856 AA037483 AW572535 AI143991 AA084581 AA033610 |
| | | AV742510 AV735788 R08336 |
| 443695 | 20416_12 | BE535598 AW204099 AW301249 AA609749 BF917914 AA775742 AV646137 AV646389 AA314747 |
| 451752 | 10408_5 | AB032997 AI141678 AW978722 BE467119 AI761408 BF727385 AW237035 AI934521 BF436248 AI479668 Z40632 AA832081 AW295901 |
| | | BF057835 BE465977 AI621269 BE465983 BF756369 N74056 AI817896 AA716567 AA934774 H62600 H09497 BF943762 BE395335 |
| | | BE883333 |
| 437834 | 294580_1 | BG110129 AW749287 BE535498 AW749299 AW749293 AW749302 AW749298 AW749291 AW749294 AW749289 AW749288 AW749296 |
| | | AA769294 AW749297 AW749295 AW749292 BE002573 |
| 446999 | 70312_1 | AA151520 AI749635 AA149436 BE172702 AW317084 AA922501 AI302818 AI147563 AA789216 AI719336 AW612978 F34536 AI971386 |
| | | AI246525 AI183312 R02554 AI360172 AA634282 AI022935 AA639461 AI086411 AI087086 AA633082 AI590029 AA856582 AW369734 |
| | | AA150042 AA877171 AA456459 AI078529 R83333 AI161298 AA056987 AI350120 H88127 AA258759 AI673598 AA454566 AI342790 |
| | | AI492606 AI159945 AI198009 AI198039 AI142751 AI141403 T81478 AW014110 AI197808 AI927796 AA534936 AA649735 AA649697 |
| | | AI349452 AA719660 AW954285 |
| 447350 | 2267324_1 | AI375572 AI480404 BF430912 T06882 |
| 438869 | 52134_1 | AF075009 R63109 R63068 |
| 418304 | 1093209_1 | BE883520 BI057842 AA215702 AA215703 AA368006 BE006876 BE066555 |
| 400277 | 170_1 | Y00281 NM_002950 BC010839 BC007995 BG675232 BM468552 AL555484 BG831516 BF035300 BG677277 BF852972 BE314901 |
| | | BF850656 AI371816 AA292474 AA375747 AA308414 BM454544 BI333370 BM049921 BI461428 BI465007 BI223401 BE856245 AW821164 |
| | | BF914775 BF914761 AU125835 BI222678 BI091137 BF340536 BM462798 BI224452 BG707915 AL569160 AA443815 AW572867 |
| | | AW363410 BF739268 BG010283 BI013120 BF818845 BF763468 AA305165 AI630370 AA039826 R24906 H02046 T96891 BF981330 |
| | | AW936510 AA478169 H04587 BG166574 BI869342 BE562482 BE539637 AA165089 AL579118 AL553699 BE044054 AW117440 AI520674 |
| | | BF435417 AW245648 AI952404 T29534 AU153459 AU152168 AW591591 AU146918 AI393187 AA478013 AU148143 AI224471 AI640728 |
| | | AI871537 AW264752 N93787 AI189357 AV756134 AI471659 AU147466 AA779206 AU149419 AU149104 AU159135 AA312221 AW445119 |
| | | AW021912 AI799771 F04407 AI285530 AI914643 AW068751 AA513325 AA164627 AA639285 AA569644 T96892 AI923594 BF439180 |
| | | BI770936 BF032438 AU154884 AA682793 AW072992 AU158815 AI884444 AL048031 AU158922 AU152546 AI695187 AL048033 AI245650 |
| | | AU148507 AW467451 BE536868 BF913001 BF062707 AL573082 AW067993 AA523354 BE886727 AI890705 AU159092 AI982693 |
| | | AI817553 AA236729 AI687858 BG163767 AI524675 AI678155 AA127100 AI762661 AU159718 AI469720 AA483627 AW131696 R26868 |
| | | AI199885 AW875614 AW938694 AW578974 BI763988 BG819168 BE874767 BG978292 BE162948 AL555483 AW189719 T56783 AI018819 |
| | | AI476552 BI492837 AI824440 BG996262 AA932887 AI380726 R79530 AA622108 AI262575 T56782 R27437 BE784153 AW129549 |
| | | AI675567 AI866759 BG987935 |
| 406685 | 0_0 | M18728 |
| 407021 | | |
| 441128 | 20932_1 | BC014072 BE328850 AI356567 AI148171 AI022165 BG149661 BF000671 AA233101 AA573721 AA447991 AW016855 AI005068 AA554071 |
| | | BF478215 AA906902 AW014761 BE905651 BE512923 BM047129 AA243852 AA232991 AA127550 AA127551 AA570256 AI473237 |
| | | BF033706 N90525 AW973623 AI359627 BG674574 BE903322 |
| 447349 | 1063443_1 | BE743847 AW809603 BM469626 AI375546 |
| 400295 | 2196_1 | NM_058173 AF414087 W72837 BF742809 AW070916 BE092421 AI905687 AA340069 BE074512 AI905623 AI905633 BG202312 W72838 |
| | | AI139456 BG218084 BE926938 BE186013 AW176044 AW291950 BG185269 BG197186 BG192597 BG183176 BG207535 AI127172 |
| | | BE815819 AI905624 R75793 BG202313 AI905837 BE815853 |
| 420111 | 256912_1 | AW967920 AA262684 AA255652 AA280911 |
| 455286 | 1149378_1 | W27935 AW887403 AW887474 BE144384 BE144386 |
| 424036 | 6226_1 | NM_033445 BC001193 AI885781 BF794032 AA476620 AA810906 AA810905 AI291244 AI885097 AI359708 AI335629 H97396 AI344589 |
| | | AA300377 AA457566 AW771833 BE465621 AI364068 AI364452 AI648505 AI918342 AI928670 AA886580 AL531029 AA886344 AI186419 |
| | | BG329096 BM045465 AL531028 BG437151 BE868021 AA179427 |
| 443845 | 507824_1 | BG394808 BE858105 AI569728 AI590084 BE223011 AW007054 AI554692 AI939398 AW014243 AI088747 AI498970 AI199622 BF115458 |
| | | BI714844 BI715424 AW135964 BG470892 BF347984 AI569769 AI424675 AI939616 BF116017 BF513472 AI828151 AI199676 AW139725 |
| | | AI475044 AI128872 |
| 427521 | 513212_1 | AW973352 BF222929 AW016853 BF059130 AI651829 BE551767 AA558414 AI339359 BF059601 AI961162 AI341422 AI206248 AI206165 |
| | | AA548736 AA768578 AI539081 AW025957 AA736837 N79575 AW594357 AA480892 |
| 424339 | 50559_1 | NM_015720 AF219137 AL534420 AL524055 AL537346 AL538442 BG765888 AL530054 AL525377 BG474596 BG473144 BE251553 |
| | | BG706099 AL538039 BG703131 BE255806 BF805256 F12128 AL566773 BI828686 BF761480 AI204971 BG818818 BI199246 AL534816 |
| | | BF529941 AA324163 AL523285 BG914330 H07952 AL534815 BE769903 AI867802 BM310135 AL533702 BE254484 BF528852 BE867462 |
| | | BE740130 AL134164 AL567115 AL533701 AL524054 AL515904 AL523284 AL568203 AL534419 BF981162 BE257148 AL561833 |
| 439574 | 689966_1 | BG532820 AW246001 AI469788 AI350090 AI446788 BE549330 W84862 AA837988 |
| 438714 | 2576235_1 | AA814859 AI582623 AA814857 |
| 416128 | 3761_6 | AK057803 BG944795 AW411505 AW949210 BI018336 AI366964 BE165417 AA173988 BF965882 AA581362 AI002701 AA340708 |
| | | BF762925 BE005389 |
| 409960 | 39576_1 | BE644758 AI082238 BF940027 AI201079 AI436035 AW275966 AI085394 AI291655 AW070441 AI474134 AI268978 AI769279 AI567682 |
| | | AA693941 BF477668 AW664149 AA283782 BF509538 AW296868 AI268977 AI168133 BM352065 AI262769 BF941976 AI056920 |
| | | AA481861 BF763697 AL565888 BM352383 AA427768 AA385346 AI186988 AA931831 AA134972 BF217480 BF111012 AA908246 |
| | | AA319849 AA318136 AL514271 BF364291 AL515057 AV702312 AA377395 AL544217 AI341000 AW193583 AI350789 AA888338 |
| | | BF945380 AW879092 AA130839 T91066 N92326 AI004389 AA078832 AL572370 W04622 BE314003 AW960808 BM360872 AA319160 |
| | | AA130778 AL514257 |
| 415889 | 12922_1 | NM_052918 AF284756 BE019093 Z42986 BE328250 BE207835 R54516 R24563 H08127 BI522616 AA551620 H07879 AI174481 BF941262 |
| | | BF222810 R54417 AW137766 AI638502 N22373 H08128 R44366 AW272405 AI675836 Z38786 N75618 F02463 AI654047 BI492031 |
| | | AW021081 |
| 407242 | | |
| 449349 | 852_3 | BG679689 AW856638 BM016319 BE327123 AW772128 BE693337 BE938262 BG013928 BF892774 BF894765 BF892588 BF890995 |
| | | BE155056 BE153569 AI934407 BE672538 AW204203 AA778306 BE502974 AI718504 AA778312 AW008224 AW299732 AI911561 |
| | | AI867457 AI521962 AI640173 AI823832 AI288888 BE467960 AI934441 AA483527 AW612103 AI802712 AW342106 AI580299 AW083293 |
| | | AI700874 AW469932 AI583726 AW302136 BE327360 AW614404 C02300 AA934834 F29737 AI262050 AA934619 AA535965 BF196507 |
| | | AA393480 BF086615 AI825386 AA009773 BI333272 T93614 AW770207 BF766665 T64641 W92713 R94110 T89897 BF086603 T93659 |
| | | AA001207 BE539257 BE541430 BE160783 BE155304 BE155454 BF891405 BF762818 |
| 442643 | 2736_1 | BC001588 BC007424 AF016369 NM_004697 BI756186 BE257019 BG500792 BI862776 AL121371 BG574833 AA703250 AA179511 |
| | | AW052006 AI280150 AI914000 AI358319 AI081204 AI082594 AA992449 AI470821 AI655744 AW237529 AA678858 AI984430 BF433055 |
| | | BE467594 BE467573 AA035630 AI289987 AI184802 AI681391 AW592416 AI138377 AI139266 AA961714 AI800163 AA418751 AW451928 |
| | | AA668676 AI273444 AI494387 BE046912 AI276555 BF196021 AA700055 AA609305 AA772596 AI635758 AI635749 H95459 AW610290 |
| | | BE464994 AA527136 BF374802 AI800175 AW195227 AI189676 BF802049 AL513632 AL554911 AL538845 BE297273 AA315321 |
| | | BM451920 BE269268 BE292835 BE018128 BG755713 BM041095 BG677009 AL039691 BF995709 BE735586 BE296453 BG393609 |
| | | BG824453 AL567522 AI745257 AW388641 AW301265 AI141144 AW029280 AU149362 AU152328 AA418960 AL121009 AI890398 |
| | | AL528748 H13050 T47086 BI000575 BF334914 BF109661 R44450 H13259 T47087 AW388646 BF305834 AL577515 BM041600 BE889299 |
| | | BF239768 |
| 407137 | | |
| 426919 | 347372_1 | BI917595 AI203314 AL041228 AV27959 D61361 D82004 BI753157 AA961066 AI990307 BF439651 AI453076 AI376075 AI014836 |
| | | AI018308 AW183530 AA393346 AA935601 AA628633 AI150282 AI028574 AI217182 AA431478 AW087473 AW900295 H50055 AL041229 |
| | | B1917726 |
| 432586 | 6633_1 | BC022881 AU150944 BG750783 AW754175 AW857737 AI911659 AI050036 AA554053 AI826259 AA568548 |
| 410507 | 4450_2 | AK027433 AF117064 NM_013319 BC004468 AI149901 AI150093 AI374696 AI566580 AA779898 BG696067 BG828923 BM051241 |
| | | BM050350 AL580560 AL558826 BG182261 BG194259 BG194258 BF036155 AI026803 AI024570 AA702281 AI566953 AW662600 |
| | | AA463546 F33147 AA357796 BE312357 AL516788 AW958856 BE730432 H85868 AA046292 BG478025 BG112231 BG763623 AA098922 |
| | | BI093481 BE746381 AW962126 AI040821 BG026983 AA355288 BE392859 AA085571 BF875433 BF316280 BG740116 BG166624 |
| 454453 | 8582_4 | BF313069 BE879305 AW752781 AW752727 AW752559 AW752578 AW752584 BF646118 AL545903 BF646115 AL525361 |
| 442961 | 60316_2 | BE966247 BE220885 BE467384 BE350135 BE672094 AI811582 AW665254 AA772731 AI283601 AA417067 AW197746 AI868357 |
| | | AI792143 AA931120 AI758506 AA843761 BE737582 AW379586 N38812 BG567321 H13257 |
| 411605 | 10026_3 | BG256892 H10532 N46614 R52610 AW977696 BM460488 W56819 BI042183 BG977498 BE767451 BF870009 BG477472 R61137 R14274 |
| | | R20259 R09686 BI838226 BF034269 AA429173 BE741829 AW867495 AI123683 AW006831 BE831162 AW452753 AV742717 W86152 |
| | | BF115102 AI633815 BF921562 AA094230 BE092587 W86151 AA526153 AI672156 BF914496 R12579 BF852352 AA699780 T57386 |
| | | BF903022 R09933 AA678298 |
| 459376 | 31010_1 | BC002465 BE254864 BG472164 BE258770 |

Pkey: Unique Eos probeset identifier number
CAT number: Gene cluster number
Accession: Genbank accession numbers

TABLE 2C

| Pkey | Ref | Strand | Nt_position |
|---|---|---|---|
| 404561 | 9795980 | Minus | 69039-70100 |
| 401451 | 6634068 | Minus | 119926-121272 |
| 401519 | 6649315 | Plus | 157315-157950 |
| 403485 | 9966528 | Plus | 2888-3001, 3198-3532, 3655-4117 |
| 401464 | 6682291 | Minus | 170688-170834 |
| 401866 | 8018106 | Plus | 73126-73623 |
| 404571 | 7249169 | Minus | 112450-112648 |
| 400528 | 6981824 | Plus | 472381-472528, 474170-474277, |
| | | | 475328-47554 |
| 404632 | 9796668 | Plus | 45096-45229 |
| 402496 | 9797769 | Minus | 8615-9103 |
| 400880 | 9931121 | Plus | 29235-29336, 36363-36580 |
| 401558 | 7139678 | Plus | 103510-104090 |
| 401203 | 9743387 | Minus | 172961-173056, 173868-173928 |
| 405366 | 2182280 | Plus | 22478-22632 |
| 405770 | 2735037 | Plus | 61057-62075 |
| 406277 | 5686030 | Minus | 4759-5490 |
| 405558 | 1621110 | Plus | 4502-4644, 5983-6083 |
| 406043 | 6758938 | Plus | 36609-37156 |
| 404580 | 6539738 | Minus | 240588-241589 |
| 404076 | 9931752 | Minus | 3848-3967 |
| 404857 | 5304923 | Plus | 111653-111816, 114925-115007 |
| 402102 | 8117771 | Minus | 174566-174740 |
| 400884 | 9958187 | Minus | 57979-58189 |
| 403006 | 5881378 | Plus | 44108-46264 |
| 404982 | 4432779 | Plus | 30375-30743, 32569-32719, |
| | | | 33698-33808 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 3A
About 216 genes upregulated in breast metastases to the brain relative to primary breast tumors

| Pkey | ExAccn | UniGeneID | UniGene Title | R1 | SEQ ID NO(s): |
|---|---|---|---|---|---|
| 422756 | AA441787 | Hs.119689 | glycoprotein hormones, alpha polypeptide | 10.29 | 1679 |
| 419875 | AA853410 | Hs.93557 | proenkephalin | 10.01 | 1365 |
| 446292 | AF081497 | Hs.279682 | Rh type C glycoprotein | 6.83 | 3743 5666 |
| 418678 | NM_001327 | Hs.167379 | cancer/testis antigen (NY-ESO-1) | 6.46 | 1226 5024 |
| 429504 | X99133 | Hs.204238 | lipocalin 2 (oncogene 24p3) (NGAL) | 5.86 | 2382 5411 |
| 407168 | R45175 | Hs.117183 | ESTs | 5.62 | 131 |
| 421948 | L42583 | Hs.334309 | keratin 6A | 5.45 | 1583 5130 |
| 415262 | H95572 | Hs.206521 | YME1 (S. cerevisiae)-like 1 | 5.30 | 919 |
| 446787 | U67167 | Hs.315 | mucin 2, intestinal/tracheal | 5.18 | 3787 5677 |
| 419078 | M93119 | Hs.89584 | insulinoma-associated 1 | 4.95 | 1272 5036 |
| 406643 | N77976 | Hs.347939 | hemoglobin, alpha 2 | 4.95 | 81 |
| 410407 | X66839 | Hs.63287 | carbonic anhydrase IX | 4.89 | 474 4846 |
| 421690 | AW162667 | Hs.106857 | calbindin 2, (29 kD, calretinin) | 4.85 | 1554 |
| 428434 | AW363590 | Hs.65551 | Homo sapiens, Similar to DNA segment, Ch | 4.65 | 2256 |
| 430294 | AI538226 | Hs.32976 | guanine nucleotide binding protein 4 | 4.59 | 2463 |
| 409178 | BE393948 | Hs.50915 | kallikrein 5 | 4.47 | 345 |
| 433272 | AB043585 | Hs.100890 | candidate mediator of the p53-dependent | 4.44 | 2752 5534 |
| 431667 | AA812573 | Hs.246787 | ESTs | 4.42 | 2581 |
| 431882 | NM_001426 | Hs.271977 | engrailed homolog 1 | 4.38 | 2612 5493 |
| 428865 | BE544095 | Hs.164960 | BarH-like homeobox 1 | 4.02 | 2314 |
| 453439 | AI572438 | Hs.32976 | guanine nucleotide binding protein 4 | 4.00 | 4406 |
| 443171 | BE281128 | Hs.9030 | TONDU | 3.93 | 3501 |
| 414166 | AW888941 | Hs.75789 | N-myc downstream regulated | 3.92 | 795 |
| 422799 | AI933199 | Hs.120911 | neurexophilin 4 | 3.91 | 1682 |
| 406690 | M29540 | Hs.220529 | carcinoembryonic antigen-related cell ad | 3.82 | 4748 86 |
| 422158 | L10343 | Hs.112341 | protease inhibitor 3, skin-derived (SKAL | 3.71 | 1610 5139 |
| 406791 | AI220684 | Hs.347939 | hemoglobin, alpha 2 | 3.70 | 99 |
| 440475 | AI807671 | Hs.24040 | potassium channel, subfamily K, member 3 | 3.59 | 3291 |
| 407014 | U38268 | | gb: Human cytochrome b pseudogene, partia | 3.56 | 118 |
| 409020 | AA062549 | Hs.21162 | retbindin | 3.53 | 324 |
| 417366 | BE185289 | Hs.1076 | small proline-rich protein 1B (cornifin) | 3.52 | 1087 |
| 457029 | AA397789 | Hs.161803 | ESTs | 3.50 | 4575 |
| 437433 | R74016 | Hs.121581 | ESTs | 3.45 | 3064 |
| 424998 | U58515 | Hs.154138 | chitinase 3-like 2 | 3.34 | 1907 5240 |
| 428342 | AI739168 | | Homo sapiens cDNA FLJ13458 fis, clone PL | 3.32 | 2244 |
| 418951 | F07809 | Hs.89506 | paired box gene 6 (aniridia, keratitis) | 3.20 | 1262 |
| 405452 | | | Target Exon | 3.19 | |
| 428093 | AW594506 | Hs.104830 | ESTs | 3.16 | 2220 |
| 443219 | AI354669 | Hs.187461 | ESTs, Weakly similar to C29149 proline-r | 3.15 | 3509 |
| 440449 | AA885430 | Hs.201925 | Homo sapiens cDNA FLJ13446 tis, clone PL | 3.15 | 3288 |
| 428648 | AF052728 | Hs.188021 | potassium voltage-gated channel, subfami | 3.14 | 2279 5369 |
| 423226 | AA323414 | Hs.146109 | ESTs, Weakly similar to T28937 hypotheti | 3.14 | 1729 |
| 429259 | AA420450 | Hs.292911 | Plakophilin | 3.14 | 2344 |
| 447946 | AI566164 | Hs.277445 | ESTs | 3.13 | 3923 |
| 413597 | AW302885 | Hs.117183 | ESTs | 3.10 | 739 |
| 401151 | | | Target Exon | 3.09 | |
| 419138 | U48508 | Hs.89631 | ryanodine receptor 1 (skeletal) | 3.08 | 1281 5039 |
| 437679 | NM_014214 | Hs.5753 | inositol(myo)-1(or 4)-monophosphatase 2 | 3.07 | 3082 5590 |
| 406947 | L10403 | Hs.3134 | DNA-binding protein amplifying expressio | 3.06 | 113 4759 |
| 425057 | AA826434 | Hs.1619 | achaete-scute complex (Drosophila) homol | 3.04 | 1915 |
| 443537 | D13305 | Hs.203 | cholecystokinin B receptor | 3.03 | 3528 5629 |
| 403364 | | | Target Exon | 3.02 | |
| 406716 | AW148546 | Hs.169476 | glyceraldehyde-3-phosphate dehydrogenase | 2.99 | 93 |
| 422997 | BE018212 | Hs.122908 | DNA replication factor | 2.96 | 1708 |
| 422168 | AA586894 | Hs.112408 | S100 calcium-binding protein A7 (psorias | 2.96 | 1612 |
| 449077 | AW262836 | Hs.252844 | ESTs | 2.95 | 4033 |
| 422010 | AA302049 | Hs.31181 | Homo sapiens cDNA: FLJ23230 fis, clone C | 2.94 | 1593 |
| 422256 | M64673 | Hs.1499 | heat shock transcription factor 1 | 2.94 | 1622 5144 |
| 426484 | AF104032 | Hs.184601 | solute carrier family 7 (cationic amino | 2.94 | 2265 5364 |
| 412560 | R24601 | | CCR4-NOT transcription complex, subunit | 2.91 | 645 |
| 447349 | AI375546 | | gb: tc23d04.x1 Soares_total_fetus_Nb2HF8_{—} | 2.91 | 3848 |
| 406016 | | | Target Exon | 2.90 | |
| 425371 | D49441 | Hs.155981 | mesothelin | 2.89 | 1957 5259 |
| 443672 | AA323362 | Hs.9667 | butyrobetaine (gamma), 2-oxoglutarate di | 2.88 | 3536 |
| 430147 | R60704 | Hs.234434 | hairy/enhancer-of-split related with YRP | 2.87 | 2447 |
| 403246 | | | Target Exon | 2.86 | |
| 424047 | AI868401 | Hs.138248 | hypothetical protein YH95C04 | 2.85 | 1795 |
| 450256 | AA286887 | Hs.24724 | MFH-amplified sequences with leucine-ric | 2.85 | 4120 |
| 452800 | U10991 | Hs.30660 | G2 protein | 2.84 | 4345 5763 |
| 435408 | H07897 | Hs.4302 | ESTs, Weakly similar to T29299 hypotheti | 2.82 | 2912 |
| 434567 | AK000600 | Hs.3972 | NeuAc-alpha-2,3-Gal-beta-1,3-GalNAc-alph | 2.81 | 2848 5550 |
| 450149 | AW969781 | Hs.132863 | Zic family member 2 (odd-paired Drosophi | 2.81 | 4106 |
| 409456 | U34962 | Hs.54473 | cardiac-specific homeo box | 2.79 | 374 4825 |
| 429986 | AF092047 | Hs.227277 | sine oculis homeobox (Drosophila) homolo | 2.79 | 2434 5431 |
| 412383 | AW947577 | | gb: RC0-MT0004-140300-031-b09 MT0004 Homo | 2.78 | 628 |
| 431130 | NM_006103 | Hs.2719 | HE4; epididymis-specific, whey-acidic pr | 2.77 | 2533 5470 |
| 445707 | AI248720 | Hs.114390 | ESTs | 2.75 | 3695 |
| 449709 | BE410592 | Hs.23918 | hypothetical protein PP5395 | 2.74 | 4077 |
| 403140 | | | Target Exon | 2.74 | |
| 453309 | AI791809 | Hs.32949 | defensin, beta 1 | 2.73 | 4393 |
| 423166 | AB035334 | Hs.144181 | ESTs | 2.72 | 1723 5172 |
| 413027 | NM_002885 | Hs.75151 | RAP1, GTPase activating protein 1 | 2.71 | 4891 690 |
| 447866 | AW444754 | Hs.202095 | ESTs | 2.70 | 3915 |
| 437044 | AL035864 | Hs.69517 | differentially expressed in Fanconi's an | 2.70 | 3031 |
| 403669 | | | Target Exon | 2.68 | |
| 420783 | AI659838 | Hs.99923 | lectin, galactoside-binding, soluble, 7 | 2.66 | 1453 |
| 406685 | M18728 | | gb: Human nonspecific crossreacting antig | 2.64 | 4745 83 |
| 405151 | | | Target Exon | 2.62 | |
| 426006 | R49031 | Hs.22627 | ESTs | 2.61 | 2019 |
| 424066 | Z99348 | Hs.112461 | ESTs, Weakly similar to I38022 hypotheti | 2.61 | 1798 |
| 424250 | AF073310 | Hs.143648 | insulin receptor substrate 2 | 2.61 | 1820 5205 |
| 407737 | R49187 | Hs.6659 | ESTs | 2.60 | 186 |
| 448296 | BE622756 | Hs.10949 | Homo sapiens cDNA FLJ14162 fis, clone NT | 2.60 | 3956 |
| 440232 | AI766925 | Hs.112554 | ESTs | 2.60 | 3272 |
| 420767 | AF072711 | Hs.99918 | carboxyl ester lipase (bile salt-stimula | 2.59 | 1452 5086 |
| 420230 | AL034344 | Hs.284186 | forkhead box C1 | 2.58 | 1398 5069 |
| 406835 | AI318327 | | gb: ta42c10.x1 NCI_CGAP_Lu25 Homo sapiens | 2.58 | 102 |
| 413063 | AL035737 | Hs.75184 | chitinase 3-like 1 (cartilage glycoprote | 2.58 | 692 |
| 443845 | AI590084 | | ESTs, Weakly similar to A47161 Mac-2-bin | 2.57 | 3560 |
| 412968 | AW500508 | Hs.75102 | alanyl-tRNA synthetase | 2.57 | 686 |
| 452786 | R61362 | Hs.106642 | ESTs, Weakly similar to T09052 hypotheti | 2.57 | 4344 |
| 425352 | NM_000939 | Hs.1897 | proopiomelanocortin (adrenocorticotropin | 2.56 | 1951 5256 |
| 419767 | W73306 | Hs.306668 | Homo sapiens cDNA FLJ14089 fis, clone MA | 2.55 | 1361 |
| 437142 | AI791617 | Hs.145068 | ESTs, Moderately similar to A46010 X-lin | 2.55 | 3036 |
| 401590 | | | Target Exon | 2.55 | |
| 407147 | D20425 | | gb: HUMGS01399 Human promyelocyte Homo sa | 2.55 | 130 |
| 422559 | AW247696 | Hs.155839 | hypothetical protein MGC12934 | 2.55 | 1656 |
| 426686 | AI362802 | Hs.171814 | parathymosin | 2.54 | 2087 |
| 412452 | AA215731 | | suppression of tumorigenicity 5 | 2.54 | 634 |
| 407242 | M18728 | | gb: Human nonspecific crossreacting antig | 2.53 | 142 4766 |
| 446342 | BE298665 | Hs.14846 | solute carrier family 7 (cationic amino | 2.53 | 3746 |
| 424286 | AA338285 | Hs.90744 | proteasome (prosome, macropain) 265 subu | 2.53 | 1824 |
| 412796 | U52426 | Hs.74597 | stromal interaction molecule 1 | 2.52 | 4886 670 |
| 419589 | AW973708 | Hs.201925 | Homo sapiens cDNA FLJ13446 fis, clone PL | 2.51 | 1336 |
| 433701 | AW445023 | Hs.15155 | ESTs | 2.49 | 2782 |
| 429538 | BE182592 | Hs.139322 | small proline-rich protein 2A | 2.48 | 2384 |
| 404645 | | | C9001365*: gi\|2921630\|gb\|AAC39612.1\| (U86 | 2.48 | |
| 422726 | U11690 | Hs.1572 | faciogenital dysplasia (Aarskog-Scott sy | 2.48 | 1677 5157 |
| 424347 | AA723883 | Hs.302446 | hypothetical protein MGC10334 | 2.47 | 1834 |
| 414580 | BE386918 | | gb: 601275386F1 NIH_MGC_20 Homo sapiens c | 2.46 | 848 |
| 439477 | W69813 | Hs.58042 | Homo sapiens GDNF family receptor alpha | 2.45 | 3210 |
| 422424 | AI186431 | Hs.296638 | prostate differentiation factor | 2.45 | 1645 |
| 434861 | AA206153 | Hs.4209 | mitochondrial ribosomal protein L37 | 2.45 | 2875 |
| 421661 | BE281303 | Hs.299148 | hypothetical protein FLJ21801 | 2.45 | 1551 |
| 406102 | | | gb: RC3-HT0273-120200-014-c07 HT0273 Homo | 2.44 | |
| 424463 | AW195353 | Hs.119903 | ESTs | 2.44 | 1850 |
| 431912 | AI660552 | | ESTs, Weakly similar to A56154 AbI subst | 2.44 | 2615 |
| 454453 | AW752781 | | hypothetical protein FLJ12614 similar to | 2.43 | 4485 |
| 406718 | AA505525 | Hs.169476 | glyceraldehyde-3-phosphate dehydrogenase | 2.43 | 94 |
| 438364 | AK000860 | Hs.6191 | hypothetical protein DKFZp762I166 | 2.42 | 3135 |
| 436608 | AA628980 | Hs.192371 | down syndrome critical region protein DS | 2.42 | 2997 |
| 428182 | BE386042 | Hs.293317 | ESTs, Weakly similar to GGC1_HUMAN G ANT | 2.42 | 2227 |
| 419648 | T73661 | Hs.91877 | thyroid hormone responsive SPOT14 (rat) | 2.42 | 1344 |
| 426067 | AW664691 | Hs.97053 | ESTs | 2.41 | 2022 |
| 437026 | AW976573 | | ESTs | 2.41 | 3029 |
| 405003 | | | Target Exon | 2.40 | |
| 429749 | AI685174 | Hs.22293 | ESTs, Weakly similar to MUC2_HUMAN MUCIN | 2.40 | 2413 |
| 413934 | U03056 | Hs.75619 | hyaluronoglucosaminidase 1 | 2.40 | 4909 764 |
| 426514 | BE616633 | Hs.170195 | bone morphogenetic protein 7 (osteogenic | 2.39 | 2073 |
| 436557 | W15573 | Hs.5027 | ESTs, Weakly similar to A47582 B-cell gr | 2.38 | 2993 |
| 406175 | | | C1002017*: gi\|6678229\|ref\|NP_033349.1\|T- | 2.38 | |
| 414719 | U66619 | Hs.71622 | SWI/SNF related, matrix associated, acti | 2.38 | 4926 869 |
| 400914 | | | ENSP00000228091*: Calcium-binding protein | 2.37 | |
| 449853 | AF006823 | Hs.24040 | potassium channel, subfamily K, member 3 | 2.36 | 4089 5724 |
| 401612 | | | C4000495: gi\|6677633\|ref\|NP_033595.1\|zin | 2.36 | |
| 457739 | AF161337 | Hs.283928 | Homo sapiens HSPC074 mRNA, partial cds | 2.36 | 4600 5815 |
| 456736 | AW248217 | Hs.1619 | achaete-scute complex (Drosophila) homol | 2.36 | 4558 |
| 407119 | AA167051 | Hs.252040 | EST, Moderately similar to ZN91_HUMAN ZI | 2.34 | 126 |
| 427715 | BE245274 | Hs.180428 | KIAA1181 protein | 2.34 | 2188 |
| 402961 | | | Homo sapiens mRNA; cDNA DKFZp761E0611 (f | 2.33 | |
| 447544 | AA401573 | Hs.288284 | hypothetical protein FLJ22378 | 2.33 | 3878 |
| 416135 | AW473656 | Hs.227277 | ESTs | 2.33 | 975 |
| 427722 | AK000123 | Hs.180479 | hypothetical protein FLJ20116 | 2.33 | 2190 5344 |
| 444590 | AA457456 | | hypothetical protein FLJ20435 | 2.32 | 3611 |
| 432886 | BE159028 | Hs.279704 | chromatin accessibility complex 1 | 2.32 | 2708 |
| 409108 | AA339443 | Hs.48793 | sialyltransferase 6 (N-acetyllacosaminid | 2.31 | 334 |
| 411410 | R20693 | Hs.69954 | laminin, gamma 3 | 2.31 | 553 |
| 403828 | | | C4000447*: gi\|7705570\|ref\|NP_038851.1\|KI | 2.31 | |
| 426350 | NM_003245 | Hs.2022 | transglutaminase 3 (E polypeptide, prote | 2.31 | 2056 5301 |
| 431745 | AW972448 | Hs.163425 | Novel FGENESH predicted cadherin repeat | 2.31 | 2595 |
| 424462 | AU076666 | Hs.148101 | serum constituent protein | 2.30 | 1849 |
| 430176 | AL161995 | Hs.234775 | neurturin | 2.30 | 2450 5438 |
| 425707 | AF115402 | Hs.11713 | E74-like factor 5 (ets domain transcript | 2.30 | 1992 5277 |
| 440313 | AL050060 | Hs.7158 | DKFZP566H073 protein | 2.30 | 3280 5605 |
| 417017 | AA976064 | Hs.180842 | ribosomal protein L13 | 2.30 | 1052 |
| 426207 | BE390657 | Hs.30026 | HSPC182 protein | 2.29 | 2038 |
| 424142 | AI678727 | Hs.75106 | clusterin (complement lysis inhibitor, S | 2.29 | 1810 |
| 440747 | AW297226 | Hs.137840 | ESTs, Moderately similar to SIX4_HUMAN H | 2.29 | 3316 |
| 421574 | AJ000152 | Hs.105924 | defensin, beta 2 | 2.28 | 1540 5117 |
| 402943 | | | C20000263: gi\|11436283\|ref\|XP_006959.1\| g | 2.28 | |
| 402160 | | | Target Exon | 2.28 | |
| 425140 | AB014567 | Hs.154740 | TBP-interacting protein | 2.28 | 1926 5247 |
| 435124 | AA725362 | Hs.120456 | ESTs | 2.28 | 2897 |
| 416733 | BE243319 | Hs.79672 | KIAA0652 gene product | 2.27 | 1018 |
| 459299 | BE094291 | Hs.155651 | hepatocyte nuclear factor 3, beta | 2.27 | 4665 |
| 452833 | BE559681 | Hs.30736 | KIAA0124 protein | 2.27 | 4351 |
| 429578 | AI969028 | Hs.99389 | ESTs | 2.27 | 2389 |
| 417900 | BE250127 | Hs.82906 | CDC20 (cell division cycle 20, S. cerevi | 2.27 | 1136 |
| 429469 | M64590 | Hs.27 | glycine dehydrogenase (decarboxylating; | 2.26 | 2374 5408 |
| 433472 | AI541246 | Hs.3343 | phosphoglycerate dehydrogenase | 2.26 | 2765 |
| 429849 | U33053 | Hs.2499 | protein kinase C-like 1 | 2.26 | 2418 5427 |
| 402463 | | | NM_014624: Homo sapiens S100 calcium-bind | 2.26 | 34 4704 |
| 408595 | AI925900 | Hs.178715 | ESTs, Weakly similar to TRHY_HUMAN TRICH | 2.26 | 282 |
| 427486 | AA974433 | | fibroblast growth factor 4 (heparin secr | 2.26 | 2156 |
| 426842 | NM_004210 | Hs.172700 | neuralized (Drosophila)-like | 2.26 | 2106 5324 |
| 442620 | C00138 | Hs.8535 | Homo sapiens mRNA for KIAA1668 protein, | 2.25 | 3456 |
| 409435 | AI810721 | Hs.95424 | ESTs | 2.25 | 370 |
| 418399 | AF131781 | Hs.84753 | hypothetical protein FLJ12442 | 2.25 | 1196 5014 |
| 411006 | AW813193 | Hs.17767 | KIAA1554 protein | 2.25 | 526 |
| 418329 | AW247430 | Hs.84152 | cystathionine-beta-synthase | 2.25 | 1186 |
| 429056 | AW138568 | Hs.104965 | ESTs | 2.25 | 2330 |
| 406840 | AW161940 | Hs.2186 | eukaryotic translation elongation factor | 2.25 | 103 |
| 410553 | AW016824 | Hs.272068 | hypothetical protein MGC14128 | 2.25 | 491 |
| 421502 | AF111856 | Hs.105039 | solute carrier family 34 (sodium phospha | 2.25 | 1527 5110 |
| 430937 | X53463 | Hs.2704 | glutathione peroxidase 2 (gastrointestin | 2.24 | 2522 5466 |
| 457485 | AW081072 | Hs.115960 | KIAA0939 protein | 2.24 | 4593 |
| 432241 | AI937060 | Hs.6298 | KIAA1151 protein | 2.24 | 2648 |
| 438821 | AA826425 | Hs.192375 | ESTs | 2.24 | 3168 |
| 453863 | X02544 | Hs.572 | orosomucoid 1 | 2.23 | 4438 5786 |
| 434844 | AF157116 | Hs.22350 | hypothetical protein LOC56757 | 2.23 | 2873 |
| 414075 | U11862 | Hs.75741 | amiloride binding protein 1 (amine oxida | 2.23 | 4913 785 |
| 417515 | L24203 | Hs.82237 | ataxia-telangiectasia group D-associated | 2.22 | 1099 4988 |
| 407792 | AI077715 | Hs.39384 | putative secreted ligand homologous to f | 2.22 | 196 |
| 430569 | AF241254 | Hs.178098 | angiotensin I converting enzyme (peptidy | 2.22 | 2496 5456 |
| 449842 | AA256233 | Hs.112529 | ESTs | 2.21 | 4087 |
| 436877 | AA931484 | Hs.121255 | ESTs, Weakly similar to T21069 hypotheti | 2.21 | 3017 |
| 421267 | BE314724 | Hs.103081 | ribosomal protein S6 kinase, 70 kD, polyp | 2.21 | 1500 |
| 448571 | AA486794 | Hs.66915 | ESTs, Weakly similar to 16.7 Kd protein [ | 2.21 | 3986 |
| 408393 | AW015318 | Hs.23165 | ESTs | 2.21 | 263 |
| 425883 | AL137708 | Hs.161031 | Homo sapiens mRNA; cDNA DKFZp434K0322 (f | 2.21 | 2009 5281 |
| 406919 | M88359 | | gb: Homo sapiens DNA-binding protein (ZNF | 2.21 | 108 4754 |
| 411261 | AW834655 | | gb: MR2-TT0014-291199-017-g11 TT0014 Homo | 2.21 | 543 |
| 432941 | W04803 | Hs.279851 | hypothetical protein FLJ10241 | 2.21 | 2713 |
| 436409 | AJ238982 | Hs.183656 | VNN3 protein | 2.20 | 2983 5579 |
| 444081 | AW593028 | Hs.175939 | ESTs | 2.20 | 3573 |
| 447472 | AW207347 | Hs.211101 | ESTs | 2.20 | 3864 |

Pkey: Unique Eos probeset identifier number
ExAccn: Exemplar Accession number, Genbank accession number
UniGeneID: UniGene number
UniGene Title: UniGene gene title
R1: 90th percentile of breast metastases to the brain Ais divided by the 90th percentile of breast tumor Als, where the 15th percentile of all normal body Als was subtracted from the numerator and denominator.
SEQ ID NO(s): SEQ ID number(s) for nucleic acid and protein sequences associated with table entry.

TABLE 3B

| Pkey | CAT number | Accession |
|---|---|---|
| 428342 | 6712_1 | AK056315 AI015524 AA724079 BI713619 AI377728 AW293682 AI928140 AI092404 AI085630 AA731340 |
| | | BM469629 AW968804 AA425658 AA769094 BF446026 AW118719 AI332765 AW500888 AW576556 AI859571 |
| | | AW499664 AW614573 AW629495 AW505314 W74704 AI356361 AI923640 AW070509 AI521500 |
| | | AL042095 AA609309 AA761319 AI381489 H45700 AA761333 AW265424 AA909524 AA635311 |
| | | AA649040 AI392620 Z40708 AI985564 AW263513 AA913892 AI693486 AW263502 AI806164 |
| | | AW291137 BI061872 BI059498 AA134476 AW084888 AA036967 AW370823 |
| | | T55263 BI002756 AA489664 BF827261 W74741 BF963166 |
| 412560 | 72553_1 | BF002870 AI003925 AI082639 AA194383 AA702993 AI767866 AL575096 BF593252 AI948584 AI678666 |
| | | BI963722 AI765219 AA620965 BE671938 AI004689 AI004690 AI990303 AI127228 BE856290 AW203978 |
| | | AI934786 AI770075 AI144132 AA812597 AI813349 AI142908 BE671242 AI208243 H82735 BF115200 |
| | | AJ345984 BE044308 F32992 AJ346047 F24958 AJ346565 AJ346456 F18071 R24502 BI830577 |
| | | BI222716 H83611 AA507760 BE463806 AA194467 AI865963 BF434962 C04894 AA813511 |
| | | AA112966 N89963 |
| 447349 | 1063443_1 | BE743847 AW809603 BM469626 AI375546 |
| 412383 | 1174261_1 | AW947574 AW947732 AW947577 AW947576 AW947733 AW947734 |
| 406685 | 0_0 | M18728 |
| 406835 | 0_0 | AI318327 AI318328 AI318495 |
| 443845 | 507824_1 | BG394808 BE858105 AI569728 AI590084 BE223011 AW007054 AI554692 AI939398 AW014243 AI088747 |
| | | AI498970 AI199622 BF115458 BI714844 BI715424 AW135964 BG470892 BF347984 AI569769 AI424675 |
| | | AI939616 BF116017 BF513472 AI828151 AI199676 AW139725 AI475044 AI128872 |
| 412452 | 71091_1 | BE796667 BF330981 BE394193 Z45547 BG490525 F35734 AA130708 AA577072 AA446587 AA215665 |
| | | AA978209 BG740729 BG746810 BE298184 AI356291 AI671975 AI818924 AV715722 AI078381 BI142391 |
| | | AI201085 AI198283 AI077572 AI694848 AW016425 BM456416 AI277223 AW771476 F26140 AA102778 |
| | | AW025780 R44726 AA761079 AI581346 AI991909 BM005939 BE537999 BG469717 AA114156 |
| | | BF437200 BE774942 |
| 414580 | 623093_1 | BG333973 BE385437 BE408833 BE387650 |
| 431912 | 610_10 | BI763666 BI517886 BI759051 AI688604 AI660552 BF588523 AW004785 AW295479 BF591117 |
| | | BF002672 BF064073 AA594441 AI380340 AI700219 AI659950 AI688540 AW296326 |
| 454453 | 8582_4 | BF313069 BE879305 AW752781 AW752727 AW752559 AW752578 AW752584 BF846118 |
| | | AL545903 BF846115 AL525361 |
| 437026 | 1240260_1 | AW976573 AA742335 AA830000 |
| 444590 | 8582_1 | BE907414 BI084902 AA907921 AI567715 AA653738 AA047537 AI554180 AI183767 AW440532 |
| | | AI806052 AI148988 AA595689 AI185031 AI174482 AI674395 AA292091 AA868833 |
| | | AI675517 AA481678 BF431759 AI698771 BE833514 AI742767 BF109855 F36782 F35533 |
| | | AU149106 AI914985 AI143516 AW022296 AW118286 AI041751 AI499755 AI198299 AA862671 |
| | | AI358871 AA160379 AA481440 AI003599 F28806 AA449176 BE871427 AA457456 |
| | | BF906432 AA722113 AA022499 BI252800 T64216 BE872273 AA579472 AA355128 |
| | | AA373128 T64403 F37911 |
| 427486 | 684159_1 | BF510715 BE673055 BE464111 AW590620 AI637939 AA404324 AW236441 AI650952 |
| | | BF056796 AA974433 |
| 411261 | 1074276_1 | AW834670 AW834476 AW834691 AW834604 AW834655 AW834623 |

Pkey: Unique Eos probeset identifier number
CAT number: Gene cluster number
Accession: Genbank accession numbers

TABLE 3C

| Pkey | Ref | Strand | Nt_position |
|---|---|---|---|
| 405452 | 7656638 | Minus | 93876-94275 |
| 401151 | 9438288 | Plus | 30848-31228 |
| 403364 | 8571785 | Plus | 120351-120465 |
| 406016 | 8272661 | Plus | 41341-41940 |
| 403246 | 7637831 | Minus | 143547-143654, 143741-143900 |
| 403140 | 9230855 | Plus | 69761-69894, 70628-70889 |
| 403669 | 7259739 | Minus | 86103-86267 |
| 405151 | 7107980 | Minus | 45826-46035 |
| 401590 | 9966320 | Minus | 33547-33649 |
| 404645 | 9796894 | Minus | 19384-20220 |
| 406102 | 9124026 | Minus | 242917-243390 |
| 405003 | 6957544 | Minus | 129031-130073 |
| 406175 | 7249177 | Minus | 31058-31165 |
| 400914 | 3779013 | Plus | 116586-116729, 117860-117986 |
| 401612 | 7705041 | Minus | 100597-100830 |
| 402961 | 9453790 | Plus | 42966-43193, 53444-53524 |
| 403828 | 9838214 | Plus | 31755-32148 |
| 402943 | 6456831 | Plus | 38467-39068 |
| 402160 | 8516165 | Plus | 166063-166354 |
| 402463 | 9796896 | Minus | 8818-8952 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 4A
About 350 genes downregulated in breast metastases to the brain relative to primary breast tumors

| Pkey | ExAccn | UniGeneID | UniGene Title | R1 | SEQ ID NO(s): |
|---|---|---|---|---|---|
| 418026 | BE379727 | Hs.83213 | fatty acid binding protein 4, adipocyte | 34.20 | 1148 |
| 428398 | AI249368 | Hs.98558 | ESTs | 29.08 | 2249 |
| 452426 | AI904823 | Hs.31297 | duodenal cytochrome b | 19.50 | 4306 |
| 441591 | AF055992 | Hs.183 | Duffy blood group | 16.67 | 3358 5617 |
| 447205 | BE617015 | Hs.11006 | ESTs, Moderately similar to T17372 plasm | 16.36 | 3829 |
| 407694 | U77594 | Hs.37682 | retinoic acid receptor responder (tazaro | 15.21 | 181 4779 |
| 447990 | BE048821 | Hs.20144 | small inducible cytokine subfamily A (Cy | 15.00 | 3933 |
| 442321 | AF207664 | Hs.8230 | a disintegrin-like and metalloprotease ( | 14.98 | 3416 5619 |
| 420202 | AL036557 | Hs.95910 | putative lymphocyte G0/G1 switch gene | 14.47 | 1394 |
| 416950 | AL049798 | Hs.80552 | dermatopontin | 14.39 | 1042 4972 |
| 447225 | R62676 | Hs.17820 | Rho-associated, coiled-coil containing p | 14.13 | 3831 |
| 422109 | S73265 | Hs.1473 | gastrin-releasing peptide | 13.57 | 1604 5138 |
| 428411 | AW291464 | Hs.10338 | ESTs | 13.45 | 2251 |
| 450701 | H39960 | Hs.288467 | hypothetical protein XP_098151 (leucine- | 12.79 | 4152 |
| 450239 | BE541781 | Hs.24697 | cytidine monophosphate-N-acetylneuramini | 12.51 | 4116 |
| 431089 | BE041395 | | ESTs, Weakly similar to unknown protein | 11.92 | |
| 453655 | AW960427 | Hs.342874 | transforming growth factor, beta recepto | 11.87 | 4421 |
| 424206 | NM_003734 | Hs.198241 | amine oxidase, copper containing 3 (vasc | 11.82 | 1815 5203 |
| 443932 | AW888222 | Hs.9973 | tensin | 11.75 | 3563 |
| 412810 | M21574 | Hs.74615 | platelet-derived growth factor receptor, | 11.35 | 4888 672 |
| 422087 | X58968 | Hs.111301 | matrix metalloproteinase 2 (gelatinase A | 11.22 | 1600 |
| 418058 | AW161552 | Hs.83381 | guanine nucleotide binding protein 11 | 11.20 | 1154 |
| 415274 | AF001548 | Hs.78344 | myosin, heavy polypeptide 11, smooth mus | 11.03 | 4944 921 |
| 451583 | AI653797 | Hs.24133 | ESTs | 10.88 | 4230 |
| 452669 | AA216363 | Hs.262958 | hypothetical protein DKFZp434B044 | 10.54 | 4332 |
| 413624 | BE177019 | Hs.75445 | SPARC-like 1 (mast9, hevin) | 10.42 | 741 |
| 442561 | NM_013450 | Hs.8383 | bromodomain adjacent to zinc finger doma | 10.20 | 3442 5621 |
| 431971 | BE274907 | Hs.77385 | myosin, light polypeptide 6, alkali, smo | 10.16 | 2622 |
| 446808 | AA703226 | Hs.16193 | Homo sapiens mRNA; cDNA DKFZp586B211 (fr | 9.97 | 3790 |
| 420105 | AW015571 | Hs.32244 | ESTs, Weakly similar to FMOD_HUMAN FIBRO | 9.97 | 1385 |
| 408741 | M73720 | Hs.646 | carboxypeptidase A3 (mast cell) | 9.77 | 300 4805 |
| 428046 | AW812795 | Hs.337534 | ESTs, Moderately similar to I38022 hypot | 9.75 | 2217 |
| 453299 | W44626 | Hs.30627 | ESTs | 9.58 | 4392 |
| 453874 | AW591783 | Hs.36131 | collagen, type XIV, alpha 1 (undulin) | 9.55 | 4440 |
| 406964 | M21305 | | FGENES predicted novel secreted protein | 9.47 | 114 4760 |
| 425701 | AA361850 | Hs.240443 | Human clone 137308 mRNA, partial cds | 9.35 | 1990 |
| 417365 | D50683 | Hs.82028 | transforming growth factor, beta recepto | 9.02 | 1086 4982 |
| 408491 | AI088063 | Hs.7882 | ESTs | 9.01 | 272 |
| 414496 | W73853 | | ESTs | 8.93 | 837 |
| 415550 | L13720 | Hs.78501 | growth arrest-specific 6 | 8.91 | 4949 936 |
| 421823 | N40850 | Hs.28625 | ESTs | 8.82 | 1568 |
| 424634 | NM_003613 | Hs.151407 | cartilage intermediate layer protein, nu | 8.77 | 1866 5222 |
| 432485 | N90866 | Hs.276770 | CDW52 antigen (CAMPATH-1 antigen) | 8.76 | 2672 |
| 422287 | F16365 | Hs.114346 | cytochrome c oxidase subunit VIIa polype | 8.66 | 1628 |
| 416931 | D45371 | Hs.80485 | adipose most abundant gene transcript 1 | 8.65 | 1039 4970 |
| 442560 | AA365042 | Hs.325531 | ESTs, Weakly similar to 2004399A chromos | 8.58 | 3441 |
| 406800 | AA505535 | | gb: nh84h10.s1 NCI_CGAP_Br1.1 Homo sapien | 8.57 | 100 |
| 413856 | D13639 | Hs.75586 | cyclin D2 | 8.55 | 4907 758 |
| 456938 | X52509 | Hs.161640 | tyrosine aminotransferase | 8.53 | 4568 5805 |
| 447371 | AA334274 | Hs.18368 | DKFZP564B0769 protein | 8.42 | 3851 |
| 453767 | AB011792 | Hs.35094 | extracellular matrix protein 2, female o | 8.34 | 4430 5782 |
| 437176 | AW176909 | Hs.42346 | calcineurin-binding protein calsarcin-1 | 8.32 | 3042 |
| 453676 | AW853745 | Hs.286035 | hypothetical protein FLJ22686 | 8.32 | 4423 |
| 414541 | BE293116 | Hs.76392 | aldehyde dehydrogenase 1 family, member | 8.04 | 842 |
| 453355 | AW295374 | Hs.31412 | myopodin | 7.91 | 4400 |
| 413190 | AA151802 | Hs.40368 | adaptor-related protein complex 1, sigma | 7.87 | 698 |
| 446141 | AW631255 | Hs.324470 | L-3-hydroxyacyl-Coenzyme A dehydrogenase | 7.77 | 3726 |
| 421296 | NM_002666 | Hs.103253 | perilipin | 7.74 | 1504 5100 |
| 430410 | AF099144 | Hs.347933 | tryptase beta 1 | 7.68 | 2484 5451 |
| 427373 | AB007972 | Hs.130760 | myosin phosphatase, target subunit 2 | 7.68 | 2149 |
| 422550 | BE297626 | Hs.296049 | microfibrillar-associated protein 4 | 7.65 | 1655 |
| 444933 | NM_016245 | Hs.12150 | retinal short-chain dehydrogenase/reduct | 7.63 | 3641 5648 |
| 420255 | NM_007289 | Hs.1298 | membrane metallo-endopeptidase (neutral | 7.60 | 1400 5070 |
| 425809 | AA370362 | Hs.57958 | EGF-TM7-latrophilin-related protein | 7.51 | 1997 |
| 449746 | AI668594 | Hs.176588 | ESTs, Weakly similar to CP4Y_HUMAN CYTOC | 7.48 | 4080 |
| 436394 | AA531187 | Hs.126705 | ESTs | 7.48 | 2982 |
| 448429 | D17408 | Hs.21223 | calponin 1, basic, smooth muscle | 7.46 | 3969 5709 |
| 427585 | D31152 | Hs.179729 | collagen, type X, alpha 1 (Schmid metaph | 7.37 | 2169 |
| 406387 | | | Target Exon | 7.37 | |
| 453180 | N46243 | Hs.110373 | ESTs, Highly similar to T42626 secreted | 7.36 | 4383 |
| 454035 | AW368993 | Hs.323748 | Homo sapiens clone CDABP0086 mRNA sequen | 7.33 | 4463 |
| 435684 | NM_001290 | Hs.4980 | LIM domain binding 2 | 7.33 | 2937 5568 |
| 416157 | NM_003243 | Hs.342874 | transforming growth factor, beta recepto | 7.32 | 4956 977 |
| 435359 | T60843 | Hs.189679 | ESTs | 7.31 | 2909 |
| 452390 | AI864142 | Hs.29288 | hypothetical protein FLJ21865 | 7.19 | 4303 |
| 421124 | AI366452 | Hs.184430 | ESTs | 7.12 | 1483 |
| 428834 | AW899713 | Hs.339315 | ESTs | 7.09 | 2309 |
| 425247 | NM_005940 | Hs.155324 | matrix metalloproteinase 11 (stromelysin | 7.08 | 1943 5253 |
| 417696 | BE241624 | Hs.82401 | CD69 antigen (p60, early T-cell activati | 6.92 | 1113 |
| 409038 | T97490 | Hs.50002 | small inducible cytokine subfamily A (Cy | 6.87 | 326 |
| 456373 | BE247706 | Hs.89751 | membrane-spanning 4-domains, subfamily A | 6.85 | 4539 |
| 411962 | AA099050 | | gb: zk85d12.r1 Soares_pregnant_uterus_NbH | 6.85 | 594 |
| 418336 | BE179882 | | glutathione peroxidase 3 (plasma) | 6.85 | 1188 |
| 435010 | N89307 | Hs.124696 | oxidoreductase UCPA | 6.83 | 2887 |
| 442895 | AI814663 | Hs.170133 | forkhead box O1A (rhabdomyosarcoma) | 6.80 | 3478 |
| 418283 | S79895 | Hs.83942 | cathepsin K (pycnodysostosis) | 6.74 | 1175 5007 |
| 431493 | AI791493 | Hs.129873 | ESTs, novel cytochrome P450 | 6.73 | 2560 |
| 434096 | AW662958 | Hs.75825 | pleiomorphic adenoma gene-like 1 | 6.72 | 2815 |
| 452093 | AA447453 | Hs.27860 | Homo sapiens mRNA; cDNA DKFZp586M0723 (f | 6.62 | 4269 |
| 423366 | Z80345 | Hs.127610 | acyl-Coenzyme A dehydrogenase, C-2 to C- | 6.61 | 1739 5177 |
| 443679 | AK001810 | Hs.9670 | hypothetical protein FLJ10948 | 6.58 | 3538 5630 |
| 436293 | AI601188 | Hs.120910 | ESTs | 6.50 | 2976 |
| 423575 | C18863 | Hs.163443 | intron of periostin (OSF-2os) | 6.49 | 1759 |
| 411764 | T40064 | Hs.71968 | Homo sapiens mRNA; cDNA DKFZp564F053 (fr | 6.47 | 575 |
| 426488 | X03350 | Hs.4 | alcohol dehydrogenase 1B (class I), beta | 6.45 | 2071 5307 |
| 412088 | AI689496 | Hs.108932 | ESTs | 6.44 | 606 |
| 435088 | NM_000481 | Hs.102 | aminomethyltransferase (glycine cleavage | 6.37 | 2894 5561 |
| 408988 | AL119844 | Hs.49476 | Homo sapiens clone TUA8 Cri-du-chat regi | 6.35 | 320 |
| 430280 | AA361258 | Hs.237868 | interleukin 7 receptor | 6.28 | 2459 |
| 418310 | AA814100 | Hs.86693 | ESTs | 6.27 | 1180 |
| 452307 | R87866 | Hs.95120 | ESTs, Weakly similar to HZHU hemoglobin | 6.25 | 4294 |
| 406801 | AW242054 | Hs.190813 | ribosomal protein L9 | 6.25 | 101 |
| 456898 | NM_001928 | Hs.155597 | D component of complement (adipsin) | 6.23 | 4566 5803 |
| 410611 | AW954134 | Hs.20924 | KIAA1628 protein | 6.22 | 497 |
| 453510 | AI699482 | Hs.42151 | ESTs | 6.19 | 4415 |
| 450954 | AI904740 | Hs.25691 | receptor (calcitonin) activity modifying | 6.18 | 4178 |
| 407828 | AW959500 | Hs.49597 | retinoic acid induced 2 | 6.10 | 203 |
| 419047 | AW952771 | Hs.90043 | ESTs | 6.10 | 1269 |
| 414005 | AA134489 | | ESTs | 6.04 | 773 |
| 418318 | U47732 | Hs.84072 | transmembrane 4 superfamily member 3 | 6.01 | 1182 |
| 421893 | NM_001078 | Hs.109225 | vascular cell adhesion molecule 1 | 6.00 | 1577 5127 |
| 418994 | AA296520 | Hs.89546 | selectin E (endothelial adhesion molecul | 5.99 | 1266 |
| 413956 | AI821351 | Hs.193133 | ESTs, Weakly similar to ALU7_HUMAN ALU S | 5.95 | 770 |
| 416030 | H15261 | Hs.21948 | ESTs | 5.93 | 967 |
| 424580 | AA446539 | Hs.339024 | ESTs, Weakly similar to A46010 X-linked | 5.89 | 1860 |
| 429697 | AW296451 | Hs.24605 | ESTs | 5.88 | 2407 |
| 445457 | AF168793 | Hs.12743 | camitine O-octanoyltransferase | 5.86 | 3676 5655 |
| 437027 | AB023235 | Hs.5400 | KIAA1018 protein | 5.85 | 3030 5583 |
| 439569 | AW602166 | Hs.222399 | CEGP1 protein | 5.83 | 3217 |
| 423024 | AA593731 | Hs.325823 | ESTs, Moderately similar to ALU5_HUMAN A | 5.82 | 1713 |
| 438564 | AA381553 | Hs.198253 | major histocompatibility complex, class | 5.82 | 3149 |
| 427605 | NM_000997 | Hs.337445 | ribosomal protein L37 | 5.78 | 2171 5340 |
| 410023 | AB017169 | Hs.57929 | slit (Drosophila) homolog 3 | 5.77 | 431 4835 |
| 410016 | AA297977 | Hs.57907 | small inducible cytokine subfamily A (Cy | 5.68 | 429 |
| 418807 | NM_004944 | Hs.88646 | deoxyribonuclease I-like 3 | 5.63 | 1242 5030 |
| 436686 | AW450205 | Hs.305890 | BCL2-like 1 | 5.59 | 3004 |
| 411988 | AA455459 | Hs.164480 | ESTs, Weakly similar to T50609 hypotheti | 5.59 | 599 |
| 407891 | AA486620 | Hs.41135 | endomucin-2 | 5.57 | 212 |
| 418658 | AW874263 | Hs.32468 | ESTs | 5.57 | 1221 |
| 427007 | NM_006283 | Hs.173159 | transforming, acidic coiled-coil contain | 5.50 | 2121 5329 |
| 442441 | AI820662 | Hs.129598 | ESTs | 5.47 | 3430 |
| 439310 | AF086120 | Hs.102793 | ESTs | 5.47 | 3198 |
| 410066 | AL117664 | Hs.58419 | DKFZP586L2024 protein | 5.46 | 438 4836 |
| 448121 | AL045714 | Hs.128653 | hypothetical protein DKFZp564F013 | 5.45 | 3945 |
| 441499 | AW298235 | Hs.101689 | ESTs | 5.43 | 3354 |
| 459297 | BE300741 | Hs.125034 | hypothetical protein FLJ13340 | 5.41 | 4664 |
| 414807 | AI738616 | Hs.77348 | hydroxyprostaglandin dehydrogenase 15-(N | 5.40 | 879 |
| 409079 | W87707 | Hs.82065 | interleukin 6 signal transducer (gp130, | 5.39 | 332 |
| 408339 | R97502 | Hs.30443 | sentrin/SUMO-specific protease | 5.39 | 257 |
| 440538 | W76332 | Hs.79107 | mitogen-activated protein kinase 14 | 5.38 | 3296 |
| 414449 | AA557660 | Hs.76152 | decorin | 5.35 | 830 |
| 452165 | R17489 | Hs.28264 | Homo sapiens mRNA; cDNA DKFZp564L0822 (f | 5.32 | 4277 |
| 447073 | AW204821 | Hs.157726 | ESTs | 5.32 | 3818 |
| 409981 | AW516695 | Hs.8438 | ESTs | 5.29 | 425 |
| 415385 | R17798 | | intron of COBW-like protein (NM_018491) | 5.28 | 928 |
| 417788 | AI436699 | Hs.84928 | nuclear transcription factor Y, beta | 5.27 | 1124 |
| 455863 | AA907305 | Hs.36475 | ESTs | 5.27 | 4522 |
| 414522 | AW518944 | Hs.76325 | Immunoglobulin J chain | 5.25 | 840 |
| 457994 | AW136239 | Hs.132922 | ESTs, Weakly similar to TI47_HUMAN CARGO | 5.22 | 4605 |
| 422994 | AW891802 | Hs.296276 | ESTs | 5.22 | 1707 |
| 420570 | AI453665 | Hs.290870 | ESTs, Weakly similar to I38588 reverse t | 5.21 | 1434 |
| 431615 | AW295859 | Hs.235860 | ESTs | 5.21 | 2576 |
| 419055 | AI365384 | Hs.11571 | Homo sapiens cDNA FLJ11570 fis, clone HE | 5.18 | 1270 |
| 451820 | AW058357 | Hs.199248 | ESTs | 5.17 | 4251 |
| 422583 | AA410506 | Hs.27973 | KIAA0874 protein | 5.17 | 1660 |
| 421932 | W51778 | Hs.323949 | kangai 1 (suppression of tumorigenicity | 5.15 | 1581 |
| 425095 | AW014160 | Hs.182585 | KIAA1276 protein | 5.14 | 1920 |
| 419490 | NM_006144 | Hs.90708 | granzyme A (granzyme 1, cytotoxic T-lymp | 5.14 | 1319 5049 |
| 426406 | AI742501 | Hs.169756 | complement component 1, s subcomponent | 5.12 | 2062 |
| 418452 | BE379749 | Hs.85201 | C-type (calcium dependent, carbohydrate- | 5.11 | 1202 |
| 437773 | U24186 | Hs.283018 | replication protein A complex 34 kd subu | 5.10 | 3090 5592 |
| 439177 | AW820275 | Hs.76611 | ESTs, Weakly similar to I38022 hypotheti | 5.10 | 3189 |
| 441233 | AA972965 | | ESTs | 5.08 | 3339 |
| 428024 | Z29067 | Hs.2236 | NIMA (never in mitosis gene a)-related k | 5.06 | 2214 5350 |
| 416585 | X54162 | Hs.79386 | leiomodin 1, smooth muscle (LMOD1) (Thy | 5.03 | 1004 4964 |
| 459587 | AA031956 | | gb: zk15e04.s1 Soares_pregnant_uterus_NbH | 5.03 | 4673 |
| 410209 | AI583661 | Hs.60548 | hypothetical protein PRO1635 | 5.02 | 455 |
| 440874 | NM_003188 | Hs.7510 | mitogen-activated protein kinase kinase | 5.02 | 3319 5614 |
| 442070 | BE244622 | Hs.8084 | hypothetical protein dJ465N24.2.1 | 5.01 | 3394 |
| 408731 | R85652 | | Homo sapiens mRNA; cDNA DKFZp434F1928 (f | 5.00 | 298 |
| 420556 | AA278300 | Hs.124292 | Homo sapiens cDNA: FLJ23123 fis, clone L | 5.00 | 1432 |
| 413200 | AA127395 | Hs.222414 | ESTs | 4.99 | 700 |
| 448141 | AI471598 | | ESTs | 4.99 | 3948 |
| 414142 | AW368397 | Hs.334485 | hemicentin (fibulin 6) | 4.98 | 792 |
| 422241 | Y00062 | Hs.170121 | protein tyrosine phosphatase, receptor t | 4.98 | 1617 5142 |
| 452683 | AI089575 | | progesterone membrane binding protein | 4.98 | 4334 |
| 421998 | R74441 | | poly(A)-binding protein, nuclear 1 | 4.96 | 1591 |
| 451287 | AK002158 | Hs.26194 | likely homolog of mouse immunity-associa | 4.95 | 4207 5742 |
| 451240 | AJ131693 | Hs.58103 | A kinase (PRKA) anchor protein (yotiao) | 4.94 | 4202 5739 |
| 427620 | NM_003705 | Hs.179866 | solute carrier family 25 (mitochondrial | 4.93 | 2172 5341 |
| 443514 | BE464288 | Hs.141937 | ESTs | 4.92 | 3527 |
| 425498 | AL096725 | Hs.289010 | DKFZP434B103 protein | 4.89 | 1971 5267 |
| 447571 | AF274863 | Hs.18889 | DKFZP434M183 protein | 4.88 | 3880 5693 |
| 452040 | AW973242 | Hs.293690 | ESTs, Weakly similar to I38022 hypotheti | 4.88 | 4265 |
| 432606 | NM_002104 | Hs.3066 | granzyme K (serine protease, granzyme 3; | 4.88 | 2683 5509 |
| 422195 | AB007903 | Hs.113082 | KIAA0443 gene product | 4.87 | 1614 5141 |
| 450293 | N36754 | Hs.171118 | hypothetical protein FLJ00026 | 4.85 | 4125 |
| 446161 | AA628206 | Hs.14125 | p53 regulated PA26 nuclear protein | 4.83 | 3729 |
| 442804 | AW300118 | Hs.131257 | ESTs | 4.83 | 3472 |
| 414061 | NM_000699 | Hs.335493 | amylase, alpha 2A; pancreatic | 4.81 | 4912 782 |
| 426310 | NM_000909 | Hs.169266 | neuropeptide Y receptor Y1 | 4.80 | 2050 5298 |
| 420286 | AI796395 | Hs.111377 | ESTs | 4.80 | 1406 |
| 434025 | AF114264 | Hs.216381 | Homo sapiens clone HH409 unknown mRNA | 4.79 | 2806 5543 |
| 436648 | R18656 | | ESTs | 4.78 | 3000 |
| 449925 | AI342493 | Hs.24192 | Homo sapiens cDNA FLJ20767 fis, clone CO | 4.78 | 4091 |
| 435573 | AI580377 | Hs.34656 | ESTs | 4.77 | 2928 |
| 400419 | AF084545 | | Target | 4.77 | 17 4689 |
| 419086 | NM_000216 | Hs.89591 | Kallmann syndrome 1 sequence | 4.77 | 1274 5037 |
| 422867 | L32137 | Hs.1584 | cartilage oligomeric matrix protein (pse | 4.76 | 1687 5160 |
| 431704 | NM_006680 | Hs.2838 | malic enzyme 3, NADP()-dependent, mitoch | 4.75 | 2586 5487 |
| 452107 | AB020681 | Hs.27973 | KIAA0874 protein | 4.74 | 4271 5755 |
| 427544 | AI767152 | Hs.181400 | ESTs, Weakly similar to I78885 serine/th | 4.72 | 2163 |
| 453143 | AA382234 | | protein tyrosine phosphatase, receptor t | 4.71 | 4378 |
| 456676 | AI870001 | Hs.334479 | ESTs, Moderately similar to KIAA1139 pro | 4.71 | 4556 |
| 442295 | AI827248 | Hs.224398 | Homo sapiens cDNA FLJ11469 fis, clone HE | 4.70 | 3412 |
| 444483 | AV649942 | | gb: AV649942 GLC Homo sapiens cDNA clone | 4.69 | 3602 |
| 430234 | N29317 | | KIAA1238 protein | 4.69 | 2457 |
| 407183 | AA358015 | | gb: EST66864 Fetal lung III Homo sapiens | 4.68 | 134 |
| 438264 | T86773 | Hs.6133 | calpain 5 | 4.68 | 3128 |
| 446564 | AB037828 | Hs.15370 | KIAA1407 protein | 4.68 | 3762 5670 |
| 401274 | | | Target Exon | 4.68 | |
| 428804 | AK000713 | Hs.193736 | hypothetical protein FLJ20706 | 4.65 | 2301 5379 |
| 437952 | D63209 | Hs.5944 | solute carrier family 11 (proton-coupled | 4.65 | 3111 |
| 424896 | Z98520 | Hs.274370 | hypothetical protein FLJ20260 | 4.65 | 1896 |
| 422092 | AB007883 | Hs.111373 | KIAA0423 protein | 4.64 | 1601 5136 |
| 451871 | AI821005 | Hs.118599 | ESTs | 4.64 | 4255 |
| 414646 | AA353776 | Hs.901 | CD48 antigen (B-cell membrane protein) | 4.64 | 857 |
| 417640 | D30857 | Hs.82353 | protein C receptor, endothelial (EPCR) | 4.63 | 1109 |
| 434975 | AA657884 | Hs.314413 | ESTs | 4.63 | 2885 |
| 445263 | H57646 | Hs.42586 | KIAA1560 protein | 4.62 | 3664 |
| 417339 | AI912592 | Hs.7882 | ESTs | 4.62 | 1082 |
| 426992 | BE244961 | Hs.343200 | FE65-LIKE 2 | 4.60 | 2118 |
| 413489 | BE144228 | | gb: MR0-HT0165-140200-009-d04 HT0165 Homo | 4.60 | 727 |
| 447391 | AI377444 | Hs.54245 | ESTs, Weakly similar to S65824 reverse t | 4.60 | 3855 |
| 412802 | U41518 | Hs.74602 | aquaporin 1 (channel-forming integral pr | 4.60 | 4887 671 |
| 451529 | AI917901 | Hs.208641 | ESTs | 4.59 | 4226 |
| 443788 | AI732643 | Hs.144151 | downstream of breast cancer antigen NY-B | 4.57 | 3551 |
| 429698 | AI685086 | Hs.26339 | ESTs, Weakly similar to S21348 probable | 4.57 | 2408 |
| 430770 | AA765694 | Hs.123296 | ESTs | 4.57 | 2512 |
| 404517 | | | Target Exon | 4.56 | |
| 444301 | AK000136 | Hs.10760 | asporin (LRR class 1) | 4.55 | 3587 5637 |
| 459247 | N46243 | Hs.110373 | ESTs, Highly similar to T42626 secreted | 4.55 | 4662 |
| 407374 | AA724738 | Hs.131034 | ESTs, Weakly similar to I78885 serine/th | 4.55 | 157 |
| 446874 | AW968304 | Hs.56156 | ESTs | 4.55 | 3798 |
| 447894 | AW204253 | Hs.21912 | ESTs | 4.54 | 3919 |
| 437984 | AA781435 | Hs.334772 | hypothetical protein FLJ13614 | 4.54 | 3113 |
| 425106 | AA398972 | Hs.18987 | Homo sapiens BAC clone RP11-505D17 from | 4.54 | 1921 |
| 433735 | AA608955 | Hs.109653 | ESTs | 4.53 | 2784 |
| 438691 | AA906288 | | ESTs | 4.53 | 3156 |
| 409062 | AL157488 | Hs.50150 | Homo sapiens mRNA; cDNA DKFZp564B182 (fr | 4.52 | 329 |
| 426128 | NM_001471 | Hs.167017 | gamma-aminobutyric acid (GABA) B recepto | 4.52 | |
| 407136 | T64896 | Hs.287420 | Homo sapiens cDNA FLJ11533 fis, clone HE | 4.51 | 127 |
| 444331 | AW193342 | Hs.24144 | ESTs | 4.50 | 3590 |
| 444213 | T79623 | Hs.263351 | ESTs | 4.49 | 3584 |
| 412584 | X54870 | Hs.74085 | DNA segment on chromosome 12 (unique) 24 | 4.49 | 4881 649 |
| 411088 | BE247593 | Hs.145053 | ESTs | 4.49 | 533 |
| 414742 | AW370946 | Hs.23457 | ESTs | 4.48 | 872 |
| 441281 | BE501247 | Hs.144084 | ESTs | 4.47 | 3342 |
| 407939 | W05608 | Hs.312679 | ESTs, Weakly similar to A49019 dynein he | 4.47 | 217 |
| 421255 | BE326214 | Hs.93813 | ESTs | 4.45 | 1497 |
| 431725 | X65724 | Hs.2839 | Norrie disease (pseudoglioma) | 4.45 | 2591 5488 |
| 420311 | AW445044 | Hs.38207 | Human DNA sequence from clone RP4-530I15 | 4.44 | 1409 |
| 406687 | M31126 | | matrix metalloproteinase 11 (stromelysin | 4.44 | 4747 85 |
| 438150 | AA037534 | Hs.342874 | transforming growth factor, beta recepto | 4.41 | 3122 |
| 413902 | AU076743 | Hs.75613 | CD36 antigen (collagen type I receptor, | 4.40 | 762 |
| 434666 | AF151103 | Hs.112259 | T cell receptor gamma locus | 4.40 | 2859 5551 |
| 436169 | AA888311 | Hs.17602 | Homo sapiens cDNA FLJ12381 fis, clone MA | 4.39 | 2966 |
| 418007 | M13509 | Hs.83169 | matrix metalloproteinase 1 (interstitial | 4.39 | 1146 4998 |
| 452973 | H88409 | Hs.40527 | ESTs | 4.39 | 4362 |
| 459501 | AA854133 | Hs.310462 | ESTs | 4.39 | 4668 |
| 447109 | X69086 | Hs.286161 | Homo sapiens cDNA FLJ13613 fis, clone PL | 4.38 | 3822 5681 |
| 413869 | NM_000878 | Hs.75596 | interleukin 2 receptor, beta | 4.37 | 4908 760 |
| 440561 | AA471379 | Hs.7277 | peroxisomal biogenesis factor 3 | 4.36 | 3300 |
| 428957 | NM_003881 | Hs.194679 | WNT1 inducible signaling pathway protein | 4.35 | 2319 5384 |
| 420517 | AB011115 | Hs.98507 | KIAA0543 protein | 4.35 | 1425 5078 |
| 458627 | AW088642 | Hs.97984 | SRY (sex determining region Y)-box 17 (S | 4.35 | 4631 |
| 411779 | AA292811 | Hs.72050 | non-metastatic cells 5, protein expresse | 4.35 | 577 |
| 431474 | AL133990 | Hs.190642 | CEGP1 protein | 4.34 | 2559 |
| 416749 | AW068550 | Hs.79732 | fibulin 1 | 4.34 | 1020 |
| 418479 | AA829976 | | mannosidase, alpha, class 1A, member 2 | 4.34 | 1205 |
| 404262 | | | ENSP00000211196: DJ137F1.2 (novel member | 4.33 | |
| 426802 | AA385182 | Hs.46699 | ESTs | 4.33 | 2101 |
| 408735 | AI654450 | Hs.281706 | Homo sapiens mRNA; cDNA DKFZp564B176 (fr | 4.33 | 299 |
| 428232 | BE272452 | Hs.183109 | monoamine oxidase A | 4.32 | 2232 |
| 418307 | U70867 | Hs.83974 | solute carrier family 21 (prostaglandin | 4.31 | 1179 5008 |
| 422959 | AV647015 | | paired immunoglobulin-like receptor beta | 4.31 | 1699 |
| 423778 | Y09267 | Hs.132821 | flavin containing monooxygenase 2 | 4.31 | 1774 5187 |
| 402458 | | | C1002064: gi\|11993050\|gb\|AAG42574.1\|AF144 | 4.31 | |
| 431992 | NM_002742 | Hs.2891 | protein kinase C, mu | 4.31 | 2624 5499 |
| 415801 | R24219 | Hs.278443 | Fc fragment of IgG, low affinity Ilb, re | 4.31 | 955 |
| 422128 | AW881145 | | gb: QV0-OT0033-010400-182-a07 OT0033 Homo | 4.31 | 1607 |
| 433793 | AW975959 | Hs.107513 | ESTs, Moderately similar to KIAA1058 pro | 4.31 | 2788 |
| 438315 | R56795 | Hs.82419 | ESTs | 4.30 | 3132 |
| 434365 | AI073378 | Hs.126793 | ESTs | 4.30 | 2834 |
| 414033 | AL079707 | Hs.207443 | hypothetical protein MGC10848 | 4.30 | 775 |
| 410387 | AI277367 | Hs.47094 | ESTs | 4.30 | 472 |
| 421712 | AK000140 | Hs.107139 | hypothetical protein | 4.29 | 1556 |
| 424789 | BE176694 | Hs.279860 | tumor protein, translationally-controlle | 4.29 | 1886 |
| 420931 | AF044197 | Hs.100431 | small inducible cytokine B subfamily (Cy | 4.29 | 1465 5091 |
| 449203 | AI634578 | Hs.282121 | ESTs | 4.29 | 4044 |
| 429165 | AW009886 | Hs.118258 | prostate cancer associated protein 1 | 4.28 | 2335 |
| 403845 | | | NM_020666*: Homo sapiens protein serine t | 4.28 | 4717 49 |
| 412116 | AW402166 | Hs.784 | Epstein-Barr virus induced gene 2 (lymph | 4.28 | 608 |
| 444649 | AW207523 | | ESTs | 4.28 | 3616 |
| 412745 | AW994221 | | gb: RC3-BN0036-250200-012-d09 BN0036 Homo | 4.28 | 665 |
| 437644 | AA748575 | Hs.136748 | lectin-like NK cell receptor | 4.28 | 3077 |
| 417317 | AW296584 | Hs.293782 | ESTs | 4.27 | 1080 |
| 419169 | AW851980 | Hs.262346 | ESTs, Weakly similar to S72482 hypotheti | 4.27 | 1284 |
| 447742 | AF113925 | Hs.19405 | caspase recruitment domain 4 | 4.26 | 3899 5699 |
| 407758 | D50915 | Hs.38365 | KIAA0125 gene product | 4.26 | 192 4782 |
| 431955 | AL133606 | Hs.272244 | hypothetical protein FLJ11142 | 4.26 | 2620 5497 |
| 407307 | H73271 | | gb: yu04d05.r1 Soares fetal liver spleen | 4.25 | 150 |
| 452235 | AL039743 | Hs.28514 | testes development-related NYD-SP21 | 4.24 | 4285 |
| 408380 | AF123050 | Hs.44532 | diubiquitin | 4.24 | 262 4795 |
| 407826 | AA128423 | Hs.40300 | calpain 3, (p94) | 4.24 | 202 |
| 422431 | AI769410 | Hs.221461 | ESTs | 4.23 | 1646 |
| 433972 | AI878910 | Hs.278670 | cisplatin resistance-associated overexpr | 4.23 | 2802 |
| 454338 | AW381251 | | gb: RC0-HT0297-301099-011-a08 HT0297 Homo | 4.23 | 4477 |
| 424377 | AF081675 | Hs.146322 | killer cell lectin-like receptor subfami | 4.22 | 1836 5210 |
| 412220 | BE350058 | Hs.36787 | chromodomain helicase DNA binding protei | 4.22 | 619 |
| 407277 | AW170035 | Hs.326736 | Homo sapiens breast cancer antigen NY-BR | 4.21 | 148 |
| 421362 | AK000050 | Hs.103853 | hypothetical protein FLJ20043 | 4.21 | 1513 5103 |
| 415054 | AI733907 | | gb: zo86h09.y5 Stratagene ovarian cancer | 4.21 | 903 |
| 427326 | AI287878 | | gb: qv23f06.x1 NCI_CGAP_Lym6 Homo sapiens | 4.21 | 2143 |
| 447241 | BE382838 | Hs.19322 | Homo sapiens, Similar to RIKEN cDNA 2010 | 4.21 | 3833 |
| 416370 | N90470 | Hs.203697 | CD38 antigen (p45) | 4.19 | 990 |
| 417437 | U52682 | Hs.82132 | interferon regulatory factor 4 | 4.19 | 1095 4985 |
| 424243 | AI949359 | Hs.143600 | ESTs, Highly similar to cis Golgi-locali | 4.18 | 1818 |
| 437275 | AW976035 | Hs.292396 | ESTs, Weakly similar to A47582 B-cell gr | 4.17 | 3054 |
| 425367 | BE271188 | Hs.155975 | protein tyrosine phosphatase, receptor t | 4.17 | 1955 |
| 411878 | AW873296 | Hs.273742 | ESTs | 4.17 | 583 |
| 446170 | H49664 | Hs.125790 | leucine-rich repeat-containing 2 | 4.17 | 3732 |
| 451872 | AI821008 | Hs.10697 | ESTs | 4.17 | 4256 |
| 400143 | | | Eos Control | 4.16 | |
| 420914 | AA281697 | Hs.334827 | gb: zt03d10.r1 NCI_CGAP_GCB1 Homo sapiens | 4.16 | 1464 |
| 417054 | AF017060 | | aldehyde oxidase 1 | 4.15 | 1058 4977 |
| 423837 | AW937063 | Hs.275150 | gb: PM3-DT0037-231299-001-g11 DT0037 Homo | 4.15 | 1778 |
| 433855 | AA834082 | Hs.307559 | ESTs | 4.15 | 2792 |
| 420061 | AW024937 | Hs.29410 | ESTs | 4.15 | 1379 |
| 429490 | AI971131 | Hs.23889 | ESTs, Weakly similar to ALU7_HUMAN ALU S | 4.15 | 2377 |
| 422226 | AW517457 | Hs.42390 | nasopharyngeal carcinoma susceptibility | 4.14 | 1616 |
| 401586 | | | Target Exon | 4.13 | |
| 414152 | NM_003248 | Hs.75774 | thrombospondin 4 | 4.12 | 4914 793 |
| 419005 | T86358 | Hs.193931 | ESTs, Weakly similar to I54374 gene NF2 | 4.12 | 1267 |
| 410088 | AA738034 | | gb: nx15e08.s1 NCI_CGAR_GC3 Homo sapiens | 4.12 | 443 |
| 453876 | AW021748 | Hs.110406 | ESTs, Weakly similar to I38022 hypotheti | 4.12 | 4441 |
| 436283 | AI480319 | Hs.120058 | ESTs | 4.12 | 2974 |
| 439673 | T53169 | Hs.9587 | Homo sapiens cDNA: FLJ22290 fis, clone H | 4.12 | 3228 |
| 443622 | AI911527 | Hs.11805 | ESTs | 4.12 | 3533 |
| 448490 | AI523897 | Hs.271692 | ESTs, Weakly similar to I38022 hypotheti | 4.12 | 3976 |
| 417355 | D13168 | Hs.82002 | endothelin receptor type B | 4.11 | 1085 4981 |
| 408776 | AA057365 | | ESTs, Weakly similar to I38022 hypotheti | 4.11 | 306 |
| 408180 | N98311 | | gb: yy68e04.r1 Soares_multiple_sclerosis_{—} | 4.11 | 242 |
| 404917 | | | Target Exon | 4.11 | |
| 453216 | AL137566 | Hs.32405 | progesterone receptor (PR) | 4.10 | 4388 |
| 431708 | AI698136 | Hs.108873 | ESTs | 4.10 | 2588 |
| 420224 | M84371 | Hs.96023 | CD19 antigen | 4.10 | 1396 5068 |
| 426486 | BE178285 | Hs.170056 | Homo sapiens mRNA; cDNA DKFZp586B0220 (f | 4.10 | 2070 |
| 428594 | BE387236 | Hs.75415 | beta-2-microglobulin | 4.10 | 2275 |
| 424874 | AA347951 | | Homo sapiens cDNA FLJ20812 fis, clone AD | 4.09 | 1894 |

Pkey: Unique Eos probeset identifier number
ExAccn: Exemplar Accession number, Genbank accession number
UniGeneID: UniGene number
UniGene Title: UniGene gene title
R1: 90th percentile of breast tumor Als divided by the 90th percentile of breast metastases to the brain Als, where the 15th percentile of all normal body tissue Als was subtracted from the numerator and denominator.
SEQ ID NO(s): SEQ ID number(s) for nucleic acid and protein sequences associated with table entry.

TABLE 4B

| Pkey | CAT number | Accession |
|---|---|---|
| 431089 | 125941_2 | BG940189 AW063489 AA715980 BF001091 BF880066 AA666102 AA621946 AA491826 |
| 414496 | 1526_1 | AK058006 BF724822 W65303 AW887764 AW023806 Z25353 AW022095 AA730973 W00417 W73819 BF982096 AI927669 AW188021 |
| | | AW770478 AI913512 AA604358 AI697341 AI691028 AI338392 AI079403 H97538 AI144448 AI253102 AI051402 AI335900 AI868132 |
| | | N28900 H98465 AF268386 AI799915 AI819228 BE048413 AW304723 AI819923 BF223106 AA155907 AW298079 BF055272 BF446804 |
| | | BF197697 W58588 BF197538 AA032180 AA992597 AW590254 AA027824 AI129369 AI131331 AI655843 AA932907 AW104493 AI150615 |
| | | BF110226 AW172271 AI312659 AA057312 BE673669 AA722984 AW104985 AI129232 AI078648 AI653086 AI703481 AW515897 AI352206 |
| | | N67076 AW297281 AI686162 AA029184 AI610743 AW772016 AI091778 W65401 AI687374 AI218085 AI765158 AI018002 AI653068 |
| | | AI335704 AI520850 AW275228 AW275204 AI420247 AA975336 AI697042 AW182235 AA736386 AI281682 AW169698 AW263325 |
| | | BE645834 AI377438 AI146706 AA613808 AA716538 BI496247 AA032248 AI698930 AI193399 N70026 H86792 AA404489 W61267 |
| | | BF447230 AA910805 AA150774 AA621907 AA902526 AI827634 AW022037 BF059000 BI496246 BG577007 BG571077 AA460779 |
| | | AW816890 AW816893 AW816891 AA029183 AA010295 H86850 T83320 BE160823 H12925 N40087 AA096372 BE160847 AW816892 |
| | | AW816889 AW816882 AW816868 AW816941 AW816578 AW816940 AW816577 AI431628 AI828113 AA033677 AA033654 AA452704 |
| | | AA317582 AA346971 BF836584 H48669 BI861605 BG925200 AA463277 H89048 AA155952 W03252 W01510 W00915 W58589 AA164519 |
| | | N24017 N24622 N27149 N70109 R43771 AA010296 W84611 H98889 H88965 AW594424 AA034139 AA065223 N99696 BG981481 N94371 AI188071 AI370541 AI754442 |
| | | AA767970 W47146 N70977 H05510 W61268 T90796 AA164518 W47244 AA150883 AA034138 BF338483 F13671 H51317 W72716 N21488 |
| | | AA148524 AI749182 W95221 W92522 R20385 |
| 406800 | 0_0 | AA505535 |
| 411962 | 2307710_1 | AA099050 AA099526 T47733 |
| 418336 | 58817_1 | AW969583 BI772505 BE179578 AI493714 AI937718 AA663709 BI868925 AW138743 AI911314 BE645538 BG911947 AI380325 AI265803 |
| | | W56175 AI658779 AI675997 AW665991 AI459263 AI420121 Z38874 AA570115 AW301008 AA216257 BF062662 BI772789 H05989 |
| | | BF085523 BI001277 |
| 414005 | 259333_1 | AW968220 AA259126 AA287352 AA279767 AI479143 AA863044 AA134489 H49266 L32048 |
| 415385 | 285_10 | N92510 AW058040 AW027717 H26334 BG619539 BG426083 Z46181 F07399 R17798 AI861887 AA419558 AU185438 BE926285 |
| | | AA382353 |
| 441233 | 2645856_1 | AA972965 AI685347 AA923446 |
| 459587 | 93128_1 | BG545629 AA031999 AA031956 |
| 408731 | 11725_2 | AF305826 BE350971 AI765355 AW172600 AI310231 BM271766 AI547292 AW612019 AI674617 AW138666 AI147629 AI147620 BF857810 |
| | | BF886300 BF885952 BF886303 BF885956 BG565497 BE670834 AA114025 BF886396 BF886928 AV750861 D62864 |
| 448141 | 2047395_1 | AI660692 AI471598 H96927 |
| 452683 | 47038_2 | BG939450 AA775472 AW058592 BE855643 BF055005 AA864765 AI278037 AI655048 AI201557 AI687448 AI143618 BE500960 W58669 |
| | | AI659870 AI089575 AI668821 AA709020 AI978936 AI338511 AA600231 AI086687 AI090569 AI146299 BF221488 AI250850 AA663309 |
| | | AA744980 AA968965 AI088224 AI474516 AW057610 BE858855 AI624190 AI161307 AI247088 AA126444 AA126743 AI125007 AI433915 |
| | | AI708238 AA663513 AI370250 AA027291 AI763349 AA454524 AI708805 AA126568 AW089710 W58670 AI202620 BE835776 AW197418 |
| | | AA126821 AI961169 BG152992 F37518 BF907190 AI932429 AI421633 AI419518 W19340 BI818890 H89569 H89568 AA057704 BG028664 |
| | | BG741413 AW803665 AW889928 BF378811 BF088437 AA027290 BF093514 AW959502 AA456193 AA330467 R19974 R21000 AA362856 |
| | | W25522 |
| 421998 | 133592_1 | BI757233 BG911321 BF351759 AW244016 AW026834 AW024260 AI420138 AA779354 AI093360 AI934858 AW151292 AI373133 AI335587 |
| | | AI969728 AA101632 BE218525 AI802114 AI783721 AA845265 AW088826 AI832852 F03967 AI611148 AI720358 AW293764 N91161 |
| | | R79192 W85852 AW771263 BG820263 BG012864 R74441 R86080 W04256 BE707244 BF899452 BE327552 BE669500 AI492388 |
| | | AI241532 BF448184 AI209012 AA886528 N70309 AW582776 BF110563 BF448329 BE326537 AW770471 BF444926 BE674147 AI793266 AI991774 AI807726 AI218667 AA301750 |
| | | R44328 |
| 436648 | 52977_1 | AJ002788 AL118666 AI381600 BE672862 AW500520 BF223709 AW593740 AA262174 AA810597 AA810596 AA810595 F09382 BF976590 |
| | | AW968002 AA262288 BF931698 AW968014 R18656 BM459356 AW794189 BF954184 Z42558 BF891641 BF963380 Z45874 F05187 |
| | | X93079 BF742651 BF742649 R51324 D80031 BI457883 F06613 Z43128 F12243 BF950830 H19040 BF950829 F06439 R14947 F06702 |
| | | R61037 R52173 R14953 R12174 R13610 H10426 R11851 T65264 R18737 |
| 453143 | 10116_6 | BG542081 BF793365 AI371013 AI147536 AW005418 AA416767 AI083516 AI698032 AA410929 AI936116 AI079893 AA747741 BF940413 |
| | | BM007681 F12285 N75819 AI971415 AA032249 AW867908 AW867914 AI520867 BG990651 AA570507 AA036654 AA063585 AA873147 |
| | | AI538117 AA382234 T66232 BE272411 AA834031 BG122734 BI769788 W55850 AW879266 |
| 444483 | 389121_1 | AV649942 AV719783 AV650843 AV720464 |
| 430234 | 1746_1 | AW025803 AL137567 AI760919 BE552289 AW082686 AA913951 BE501313 AV756373 BE048863 AA828185 BG151502 AI655583 |
| | | AW473377 AI949888 AA453495 AW130287 AI222766 AI862122 AI309288 AL038626 AA714749 AI719007 W95486 N29317 AW589706 |
| | | AA905486 AI744057 AA759318 AI084950 AI273294 BF835579 AW235310 AI914478 BF887920 R21864 AI479541 AW189671 AW235752 |
| | | AI762157 BG698714 AA461269 AA460813 W95524 AA301369 AI760649 BF854009 AW303856 H42831 AV737305 H03702 R22396 R65961 |
| 413489 | 1517623_1 | BE144228 BE144291 |
| 438691 | 2575806_1 | AA829941 AA906288 AI914939 AA814353 |
| 406687 | 0_0 | M31126 |
| 418479 | 175360_1 | BF966791 BG564455 BE672212 AI151416 AI566231 AI417585 AI378391 AA236264 AI337574 AI346166 AA406590 AA748618 AW771957 |
| | | AA478626 AW338072 AI889444 AI810315 BE503662 BG231886 AI888230 AI289102 BF594638 AW074094 AW512456 AA832229 |
| | | AI056108 AI025868 AI245806 D61957 AI093841 AI721013 AI597594 AA993022 AI128620 AI285106 W37459 W35410 N90037 AA890323 |
| | | R39943 AI468741 AA829976 AA479201 AI539018 AA875875 AA448827 AW779493 Z39056 H84925 AA223923 AW517592 AI804400 |
| | | AA911882 BM353143 D62885 AI457883 AI880626 R31694 R42772 R68804 R44147 R71463 AV742540 BF966987 |
| 422959 | MH905_3 | AJ400845 AI954159 AL041618 AI028269 AA769325 AW780241 AW129462 AI271476 BF798303 AA836991 AW273346 AI436321 AI375545 |
| | | AL040967 AA889495 AI922524 AA598667 AA423804 AL040910 N80292 AI954063 AI923968 AI400578 AA748499 BE677845 AW020788 |
| | | AA860230 AW519209 AA767391 AI860419 AA476935 AW452389 AI017695 AA806940 BI497005 AI051533 AI650706 AI811516 AA609569 |
| | | AI439198 BF430946 AA749268 AI624860 AI784422 BI491753 AI206880 BE671796 AI431957 AI187038 AI678429 AI273421 AA897667 |
| | | AA586499 BE241923 AF161081 NM_013440 BE073169 AI700673 AV699081 AV684786 AV688081 AV689220 AV689216 AA132636 |
| | | BF086186 BF917106 AV762653 BI064033 BE168145 AA778650 AI984255 W69468 AA132452 N53166 AI949278 AW168519 F28686 |
| | | AA908333 F37181 BI002729 |
| 422128 | 17516_16 | BI261864 BI036453 BI023096 BI023388 AA331991 AV721898 AW881145 AA490718 M85637 T06067 AV761102 BI023091 BI022906 |
| 444649 | 630074_1 | AI184631 AW138226 AI917315 AI825123 AI651395 AI636782 AI990399 AW207523 |
| 412745 | 1246023_1 | AW994221 AW994211 BE075786 AW994378 |
| 407307 | 1984287_1 | H73271 R96266 H73959 R96214 |
| 454338 | 788429_−2 | AW381251 |
| 415054 | 3070732_1 | AI732614 AA159708 AI733907 |
| 427326 | 565_21 | Y10529 BF062364 BE501015 AA758739 AF359418 AF359419 BM021254 AF359416 BI087915 BG995764 BF345274 BG431056 AI804160 |
| | | AI287878 AA400787 |
| 400143 | 11259_1 | BC004324 NM_001020 BC007977 M60854 BM050628 BG829809 BE385504 BG744451 BI826914 BE440007 BI260656 BE395117 |
| | | BE389334 BE255792 BI194169 BI668218 BI194376 BG716213 BG714408 BE392513 AV722219 AW328077 BM424171 BI828267 |
| | | AW958606 BG831252 BE392943 BE394033 BI858915 BI668334 BE621019 BG706995 BE791985 BF967484 BI193635 BG761859 |
| | | BM466537 BG747165 BG827488 AI133550 BM011511 BI227282 BG489212 BG478388 BE727789 BI160880 BG831707 BG324692 |
| | | BM470427 BI083889 BG831605 BG754114 BG420536 BF308210 BE384213 BG832271 BG828032 BG481641 BF205675 BE899041 |
| | | BE271558 BI193807 BI159866 BG473786 BG397178 BI194428 BI117210 BG768326 BG759507 BF975645 BF343657 BM020598 |
| | | BG831082 BG829943 BG829501 BF306557 BE562511 BM050145 BM017978 BI193934 BI160764 BI160371 BG754991 BF973348 |
| | | BF663234 BF032537 BE388168 BM009051 BI192794 BG831002 BG830459 BG764737 BG761808 BG481705 BG104314 BM464565 |
| | | BI261500 BG831857 BG831684 BG829852 BG765030 BG760419 BG760268 BG749762 BG480900 BG419627 BG248771 BF975542 |
| | | BM042233 BI161149 BG831302 BG830033 BG829626 BM050064 BI193014 BI161360 BG822729 BG110091 BG106500 BI258369 |
| | | BG831982 BM458301 BM019513 BI161350 BI114178 BG481969 BG474870 BF974048 BF971122 BE741405 BE395269 BG832027 |
| | | BG831469 BG490895 BM413638 BG943529 BG831012 BG829471 BG686284 BG337575 BG336551 BF206677 BI258301 BI160946 |
| | | BG105893 BF183072 BM459542 BI193881 BG832043 BG831323 BI194545 BI160968 BG755930 BG706018 BE743865 BM465145 |
| | | BG831227 BG774290 BF683451 BE907161 BM045391 BI194396 BI161269 BG747091 BG546643 BF984863 BI160206 BI226402 BI226336 |
| | | AW328236 BG339458 BF972634 BE909808 BI160988 BI160251 BG828764 BG826860 BG758360 BF568228 BI818282 BI457127 |
| | | BG831491 BG759864 AI830010 BF568381 BE907238 BI161172 BI116773 BG827153 BG825088 BG335419 BG109404 AI929068 |
| | | BE906354 BE408564 BM045000 BG339617 BG282794 BG335767 BE907263 BF568921 BG829961 BG479305 BG260397 AI922228 |
| | | BE301975 AW516055 BG480919 BG480626 AW196817 BG336261 BE906157 BE395717 BE391427 BI192954 BG829757 BG476379 |
| | | BE301536 BE394727 BE257695 BE905344 AI433577 BE894416 BE886992 BE409223 BF034756 BE904077 BG830886 BE909153 |
| | | BE907998 BE395767 AI871751 BE744523 BI192663 BG831669 AI000225 BE743836 BE272515 AA628078 BM463802 BE393375 |
| | | BE393033 AW170187 BE730961 BE395410 BE744572 BE392297 BE391448 BE390780 BE388821 BE258477 BE905970 BE901567 |
| | | BE898833 BE880326 BF726889 BE910504 BE390753 BE390131 AA650542 BE744156 BE394125 BE742207 BE395265 BE392942 |
| | | BE894336 BE378222 BE906926 BE904650 BE393704 BE620999 BE515162 BE378753 BE272370 BE907458 BE612801 BE392484 |
| | | BE907636 BE907353 BE910491 BE909796 BE905331 AW248173 AI683576 BE908826 BE620180 BF037570 BE908312 BE615015 |
| | | BE256977 BE746875 BE394133 BE391478 BE910068 BE907185 BE742109 AA995746 BE561195 BE908825 BE906472 BE906509 |
| | | BE906017 BE910442 BE514657 BI261969 BE741707 BE392216 BM042793 BF570283 BI262119 BE395707 BE378298 AW327827 |
| | | BE394422 BF569178 BE263240 AI700512 BG830290 BF569308 BF569156 BI194587 BE390831 BG745096 AI681675 BE395674 AA136372 |
| | | BE279892 AA442822 BE384898 AA313519 AI878866 AA305904 F33366 BE394852 F29153 F33618 AI133637 AA300009 F34063 F29455 |
| | | AU099691 AI905085 AI906656 AA343249 BE388691 AW404280 AA379888 F29022 BF089981 F31013 F24305 |
| 417054 | 12405_2 | BG533564 BG618564 AW296119 AI269233 BF508328 AW364777 AW292258 AA371049 AI452471 AI092522 BG618376 AL049080 |
| | | AA631068 BG564643 T53833 AV702544 BG533452 AV05004 AA588281 T28665 BG569026 AV646874 AV647253 AV647455 AV647749 |
| | | BI759444 AV652457 AV695354 AV696010 AV697248 BG617586 AV722549 AI435836 AI590676 AI245019 AW338243 AA530898 D52191 |
| | | AI435352 D57473 BG566952 AI420505 AA035245 AV704972 BG564113 AI439237 AI287456 AV695686 AA349017 |
| 410088 | 1241437_1 | AW977844 AA738034 AA081561 |
| 408776 | 106302_1 | BF057799 BE218747 T15720 AA057365 AI811370 |
| 408180 | 640212_1 | N98311 BE160207 AW168088 |
| 424874 | 11607_6 | AI688463 BF959633 BF897027 AA883123 |

Pkey: Unique Eos probeset identifier number
CAT number: Gene cluster number
Accession: Genbank accession numbers

TABLE 4C

| Pkey | Ref | Strand | Nt_position |
|---|---|---|---|
| 406387 | 9256180 | Plus | 116229-116371, 117512-117651 |
| 401274 | 8954206 | Minus | 111258-111378 |
| 404517 | 8151983 | Minus | 92340-92443 |
| 404262 | 9367893 | Minus | 69642-69807, 70646-70812 |
| 402458 | 9796782 | Plus | 170479-171134 |
| 403845 | 9959258 | Minus | 87151-87288, 87539-87633, |
| | | | 88216-88382, 8847 |
| 401586 | 9838242 | Minus | 93974-94099 |
| 404917 | 7341851 | Plus | 49330-49498 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 5A
About 489 genes downregulated in breast metastases to the brain relative to normal breast tissue

| Pkey | ExAccn | UniGeneID | UniGene Title | R1 | SEQ ID NO(s): |
|---|---|---|---|---|---|
| 418026 | BE379727 | Hs.83213 | fatty acid binding protein 4, adipocyte | 94.34 | 1148 |
| 447205 | BE617015 | Hs.11006 | ESTs, Moderately similar to T17372 plasm | 75.45 | 3829 |
| 420202 | AL036557 | Hs.95910 | putative lymphocyte G0/G1 switch gene | 51.29 | 1394 |
| 416950 | AL049798 | Hs.80552 | dermatopontin | 41.57 | 1042 4972 |
| 410677 | NM_003278 | Hs.65424 | tetranectin (plasminogen-binding protein | 40.07 | 4852 504 |
| 441591 | AF055992 | Hs.183 | Duffy blood group | 39.13 | 3358 5617 |
| 453655 | AW960427 | Hs.342874 | transforming growth factor, beta recepto | 35.85 | 4421 |
| 426488 | X03350 | Hs.4 | alcohol dehydrogenase 1B (class I), beta | 31.80 | 2071 5307 |
| 443932 | AW888222 | Hs.9973 | tensin | 31.62 | 3563 |
| 447990 | BE048821 | Hs.20144 | small inducible cytokine subfamily A (Cy | 31.16 | 3933 |
| 454059 | NM_003154 | Hs.37048 | statherin | 30.14 | 4466 5793 |
| 415274 | AF001548 | Hs.78344 | myosin, heavy polypeptide 11, smooth mus | 30.07 | 4944 921 |
| 416931 | D45371 | Hs.80485 | adipose most abundant gene transcript 1 | 29.91 | 1039 4970 |
| 442321 | AF207664 | Hs.8230 | a disintegrin-like and metalloprotease ( | 29.18 | 3416 5619 |
| 414541 | BE293116 | Hs.76392 | aldehyde dehydrogenase 1 family, member | 28.50 | 842 |
| 424206 | NM_003734 | Hs.198241 | amine oxidase, copper containing 3 (vasc | 28.07 | 1815 5203 |
| 421998 | R74441 | | poly(A)-binding protein, nuclear 1 | 27.35 | 1591 |
| 428232 | BE272452 | Hs.183109 | monoamine oxidase A | 27.07 | 2232 |
| 410635 | D58863 | Hs.347963 | chorionic somatomammotropin hormone 1 (p | 26.61 | 500 |
| 418058 | AW161552 | Hs.83381 | guanine nucleotide binding protein 11 | 25.80 | 1154 |
| 416157 | NM_003243 | Hs.342874 | transforming growth factor, beta recepto | 25.08 | 4956 977 |
| 443514 | BE464288 | Hs.141937 | ESTs | 24.80 | 3527 |
| 411939 | AI365585 | Hs.146246 | ESTs | 24.50 | 587 |
| 445263 | H57646 | Hs.42586 | KIAA1560 protein | 23.85 | 3664 |
| 422287 | F16365 | Hs.114346 | cytochrome c oxidase subunit VIIa polype | 23.37 | 1628 |
| 412810 | M21574 | Hs.74615 | platelet-derived growth factor receptor, | 21.92 | 4888 672 |
| 428398 | AI249368 | Hs.98558 | ESTs | 21.53 | 2249 |
| 421296 | NM_002666 | Hs.103253 | perilipin | 21.52 | 1504 5100 |
| 417365 | D50683 | Hs.82028 | transforming growth factor, beta recepto | 21.33 | 1086 4982 |
| 407694 | U77594 | Hs.37682 | retinoic acid receptor responder (tazaro | 20.91 | 181 4779 |
| 412047 | AA934589 | Hs.49696 | ESTs | 20.20 | 605 |
| 411962 | AA099050 | | gb: zk85d12.r1 Soares_pregnant_uterus_NbH | 20.05 | 594 |
| 452426 | AI904823 | Hs.31297 | duodenal cytochrome b | 20.04 | 4306 |
| 422550 | BE297626 | Hs.296049 | microfibrillar-associated protein 4 | 19.29 | 1655 |
| 435684 | NM_001290 | Hs.4980 | LIM domain binding 2 | 19.08 | 2937 5568 |
| 414496 | W73853 | | ESTs | 18.40 | 837 |
| 456898 | NM_001928 | Hs.155597 | D component of complement (adipsin) | 18.38 | 4566 5803 |
| 412442 | AI983730 | Hs.26530 | serum deprivation response (phosphatidyl | 18.35 | 631 |
| 423024 | AA593731 | Hs.325823 | ESTs, Moderately similar to ALU5_HUMAN A | 18.03 | 1713 |
| 407744 | AB020629 | Hs.38095 | ATP-binding cassette, sub-family A (ABC1 | 17.87 | 187 4781 |
| 430310 | U60115 | Hs.239069 | four and a half LIM domains 1 | 17.77 | 2468 5443 |
| 446808 | AA703226 | Hs.16193 | Homo sapiens mRNA; cDNA DKFZp586B211 (fr | 17.70 | 3790 |
| 428848 | NM_000230 | Hs.194236 | leptin (murine obesity homolog) | 17.24 | 2312 5381 |
| 413624 | BE177019 | Hs.75445 | SPARC-like 1 (mast9, hevin) | 16.55 | 741 |
| 453355 | AW295374 | Hs.31412 | myopodin | 16.33 | 4400 |
| 453767 | AB011792 | Hs.35094 | extracellular matrix protein 2, female o | 16.15 | 4430 5782 |
| 423778 | Y09267 | Hs.132821 | flavin containing monooxygenase 2 | 16.03 | 1774 5187 |
| 410023 | AB017169 | Hs.57929 | slit (Drosophila) homolog 3 | 15.92 | 431 4835 |
| 443060 | D78874 | Hs.8944 | procollagen C-endopeptidase enhancer 2 | 15.91 | 3492 |
| 435088 | NM_000481 | Hs.102 | aminomethyltransferase (glycine cleavage | 15.75 | 2894 5561 |
| 429350 | AI754634 | Hs.131987 | ESTs | 15.72 | 2358 |
| 416585 | X54162 | Hs.79386 | leiomodin 1, smooth muscle (LMOD1) (Thy | 15.28 | 1004 4964 |
| 446141 | AW631255 | Hs.324470 | L-3-hydroxyacyl-Coenzyme A dehydrogenase | 15.16 | 3726 |
| 453676 | AW853745 | Hs.286035 | hypothetical protein FLJ22686 | 15.13 | 4423 |
| 442895 | AI814663 | Hs.170133 | forkhead box O1A (rhabdomyosarcoma) | 15.02 | 3478 |
| 443679 | AK001810 | Hs.9670 | hypothetical protein FLJ10948 | 15.01 | 3538 5630 |
| 425809 | AA370362 | Hs.57958 | EGF-TM7-latrophilin-related protein | 15.00 | 1997 |
| 427373 | AB007972 | Hs.130760 | myosin phosphatase, target subunit 2 | 14.95 | 2149 |
| 407102 | AA007629 | | glycerol-3-phosphate dehydrogenase 1 (so | 14.94 | 123 |
| 451533 | NM_004657 | Hs.26530 | serum deprivation response (phosphatidyl | 14.94 | 4227 5745 |
| 420105 | AW015571 | Hs.32244 | ESTs, Weakly similar to FMOD_HUMAN FIBRO | 14.77 | 1385 |
| 447371 | AA334274 | Hs.18368 | DKFZP564B0769 protein | 14.74 | 3851 |
| 414290 | AI568801 | Hs.71721 | ESTs | 14.51 | 808 |
| 424098 | AF077374 | Hs.139322 | small proline-rich protein 3 | 14.45 | 1804 5199 |
| 413902 | AU076743 | Hs.75613 | CD36 antigen (collagen type I receptor, | 14.33 | 762 |
| 417852 | AJ250562 | Hs.82749 | transmembrane 4 superfamily member 2 | 14.30 | 1130 4994 |
| 431971 | BE274907 | Hs.77385 | myosin, light polypeptide 6, alkali, smo | 13.84 | 2622 |
| 427007 | NM_006283 | Hs.173159 | transforming, acidic coiled-coil contain | 13.83 | 2121 5329 |
| 420255 | NM_007289 | Hs.1298 | membrane metallo-endopeptidase (neutral | 13.76 | 1400 5070 |
| 447225 | R62676 | Hs.17820 | Rho-associated, coiled-coil containing p | 13.75 | 3831 |
| 448429 | D17408 | Hs.21223 | calponin 1, basic, smooth muscle | 13.66 | 3969 5709 |
| 452123 | AI267615 | Hs.38022 | ESTs | 13.20 | 4273 |
| 414033 | AL079707 | Hs.207443 | hypothetical protein MGC10848 | 13.12 | 775 |
| 444933 | NM_016245 | Hs.12150 | retinal short-chain dehydrogenase/reduct | 13.07 | 3641 5648 |
| 454229 | AW957744 | Hs.278469 | lacrimal proline rich protein | 12.83 | 4473 |
| 425280 | U31519 | Hs.1872 | phosphoenolpyruvate carboxykinase 1 (sol | 12.72 | 1947 5254 |
| 422195 | AB007903 | Hs.113082 | KIAA0443 gene product | 12.58 | 1614 5141 |
| 403593 | | | Target Exon | 12.52 | |
| 409882 | AJ243191 | Hs.56874 | heat shock 27 kD protein family, member 7 | 12.22 | 412 4833 |
| 437275 | AW976035 | Hs.292396 | ESTs, Weakly similar to A47582 B-cell gr | 12.03 | 3054 |
| 406800 | AA505535 | | gb: nh84h10.s1 NCI_CGAP_Br1.1 Homo sapien | 11.99 | 100 |
| 450239 | BE541781 | Hs.24697 | cytidine monophosphate-N-acetylneuramini | 11.86 | 4116 |
| 422087 | X58968 | Hs.111301 | matrix metalloproteinase 2 (gelatinase A | 11.80 | 1600 |
| 435010 | N89307 | Hs.124696 | oxidoreductase UCPA | 11.80 | 2887 |
| 453874 | AW591783 | Hs.36131 | collagen, type XIV, alpha 1 (undulin) | 11.72 | 4440 |
| 412295 | AW088826 | | poly(A)-binding protein, nuclear 1 | 11.67 | 622 |
| 427980 | AA418305 | Hs.303205 | EST | 11.65 | 2209 |
| 441499 | AW298235 | Hs.101689 | ESTs | 11.60 | 3354 |
| 446500 | U78093 | Hs.15154 | sushi-repeat-containing protein, X chrom | 11.49 | 3756 5668 |
| 453299 | W44626 | Hs.30627 | ESTs | 11.48 | 4392 |
| 419271 | N34901 | Hs.238532 | ESTs | 11.40 | 1296 |
| 416729 | U46165 | Hs.1027 | Ras-related associated with diabetes | 11.39 | 1016 4967 |
| 407570 | Z19002 | Hs.37096 | zinc finger protein 145 (Kruppel-like, e | 11.37 | 169 4775 |
| 426928 | AF037062 | Hs.172914 | retinol dehydrogenase 5 (11-cis and 9-ci | 11.35 | 2113 5327 |
| 433633 | AI880516 | Hs.84630 | ESTs, Weakly similar to 2004399A chromos | 11.33 | 2775 |
| 452669 | AA216363 | Hs.262958 | hypothetical protein DKFZp434B044 | 11.33 | 4332 |
| 413956 | AI821351 | Hs.193133 | ESTs, Weakly similar to ALU7_HUMAN ALU S | 11.29 | 770 |
| 407099 | M94891 | Hs.173609 | pregnancy specific beta-1-glycoprotein 4 | 11.28 | 121 4764 |
| 415447 | Z97171 | Hs.78454 | myocilin, trabecular meshwork inducible | 11.09 | 4947 932 |
| 451287 | AK002158 | Hs.26194 | likely homolog of mouse immunity-associa | 11.03 | 4207 5742 |
| 442561 | NM_013450 | Hs.8383 | bromodomain adjacent to zinc finger doma | 11.03 | 3442 5621 |
| 409981 | AW516695 | Hs.8438 | ESTs | 10.93 | 425 |
| 453180 | N46243 | Hs.110373 | ESTs, Highly similar to T42626 secreted | 10.88 | 4383 |
| 407891 | AA486620 | Hs.41135 | endomucin-2 | 10.87 | 212 |
| 406801 | AW242054 | Hs.190813 | ribosomal protein L9 | 10.81 | 101 |
| 410545 | U32324 | Hs.64310 | interleukin 11 receptor, alpha | 10.79 | 4850 490 |
| 442498 | U54617 | Hs.8364 | Homo sapiens pyruvate dehydrogenase kina | 10.76 | 3435 5620 |
| 425095 | AW014160 | Hs.182585 | KIAA1276 protein | 10.67 | 1920 |
| 449101 | AA205847 | Hs.23016 | G protein-coupled receptor | 10.48 | 4035 |
| 415550 | L13720 | Hs.78501 | growth arrest-specific 6 | 10.41 | 4949 936 |
| 444562 | AA186715 | Hs.336429 | RIKEN cDNA 9130422N19 gene | 10.40 | 3607 |
| 428043 | T92248 | Hs.2240 | uteroglobin | 10.36 | 2216 |
| 447261 | NM_006691 | Hs.17917 | extracellular link domain-containing 1 | 10.33 | 3836 5685 |
| 410209 | AI583661 | Hs.60548 | hypothetical protein PRO1635 | 10.29 | 455 |
| 425078 | NM_002599 | Hs.154437 | phosphodiesterase 2A, cGMP-stimulated | 10.28 | 1917 5245 |
| 444769 | AI191650 | Hs.221436 | ESTs | 10.26 | 3626 |
| 447726 | AL137638 | Hs.19368 | matrilin 2 | 10.23 | 3896 5697 |
| 420225 | AW243046 | Hs.282076 | Homo sapiens mRNA for KIAA1650 protein, | 10.17 | 1397 |
| 411988 | AA455459 | Hs.164480 | ESTs, Weakly similar to T50609 hypotheti | 10.17 | 599 |
| 425887 | AL049443 | Hs.161283 | Homo sapiens mRNA; cDNA DKFZp586N2020 (f | 10.15 | 2010 |
| 456063 | NM_006744 | Hs.76461 | retinol-binding protein 4, interstitial | 10.03 | 4528 5797 |
| 458627 | AW088642 | Hs.97984 | SRY (sex determining region Y)-box 17 (S | 10.00 | 4631 |
| 428412 | AA428240 | Hs.126083 | ESTs | 9.97 | 2252 |
| 410016 | AA297977 | Hs.57907 | small inducible cytokine subfamily A (Cy | 9.90 | 429 |
| 414449 | AA557660 | Hs.76152 | decorin | 9.88 | 830 |
| 447299 | AF043897 | Hs.18075 | chromosome 9 open reading frame 3 | 9.82 | 3840 |
| 421823 | N40850 | Hs.28625 | ESTs | 9.78 | 1568 |
| 421218 | NM_000499 | Hs.72912 | cytochrome P450, subfamily I (aromatic c | 9.72 | 1490 5098 |
| 419119 | AA583543 | Hs.32135 | ESTs | 9.70 | 1278 |
| 457056 | NM_005534 | Hs.177559 | interferon gamma receptor 2 (interferon | 9.65 | 4576 5809 |
| 423366 | Z80345 | Hs.127610 | acyl-Coenzyme A dehydrogenase, C-2 to C- | 9.63 | 1739 5177 |
| 417511 | AL049176 | Hs.82223 | chordin-like | 9.60 | 1098 4987 |
| 427523 | BE242779 | Hs.179526 | upregulated by 1,25-dihydroxyvitamin D-3 | 9.51 | 2160 |
| 434975 | AA657884 | Hs.314413 | ESTs | 9.49 | 2885 |
| 452816 | AA131789 | Hs.61509 | ESTs | 9.47 | 4346 |
| 443793 | AA045290 | Hs.25930 | ESTs, Weakly similar to 2109260A B cell | 9.47 | 3554 |
| 441281 | BE501247 | Hs.144084 | ESTs | 9.44 | 3342 |
| 408503 | AW119059 | | ESTs, Weakly similar to T12552 hypotheti | 9.40 | 273 |
| 412000 | AW576555 | Hs.15780 | ATP-binding cassette, sub-family A (ABC1 | 9.38 | 600 |
| 452307 | R87866 | Hs.95120 | ESTs, Weakly similar to HZHU hemoglobin | 9.36 | 4294 |
| 404246 | | | Target Exon | 9.33 | |
| 436394 | AA531187 | Hs.126705 | ESTs | 9.28 | 2982 |
| 431048 | R50253 | Hs.249129 | cell death-inducing DFFA-like effector a | 9.21 | 2526 |
| 437176 | AW176909 | Hs.42346 | calcineurin-binding protein calsarcin-1 | 9.18 | 3042 |
| 444567 | AV654020 | | ESTs, Weakly similar to T26686 hypotheti | 9.18 | 3609 |
| 448274 | AI268097 | Hs.67317 | Homo sapiens cDNA FLJ11775 fis, clone HE | 9.17 | 3954 |
| 428769 | AW207175 | Hs.106771 | ESTs | 9.15 | 2293 |
| 449925 | AI342493 | Hs.24192 | Homo sapiens cDNA FLJ20767 fis, clone CO | 9.11 | 4091 |
| 425498 | AL096725 | Hs.289010 | DKFZP434B103 protein | 9.10 | 1971 5267 |
| 438150 | AA037534 | Hs.342874 | transforming growth factor, beta recepto | 9.06 | 3122 |
| 420174 | AI824144 | Hs.199749 | ESTs | 9.02 | 1391 |
| 410066 | AL117664 | Hs.58419 | DKFZP586L2024 protein | 8.98 | 438 4836 |
| 428024 | Z29067 | Hs.2236 | NIMA (never in mitosis gene a)-related k | 8.96 | 2214 5350 |
| 419407 | AW410377 | Hs.41502 | hypothetical protein FLJ21276 | 8.92 | 1311 |
| 453876 | AW021748 | Hs.110406 | ESTs, Weakly similar to I38022 hypotheti | 8.90 | 4441 |
| 445107 | AI208121 | Hs.147313 | ESTs, Weakly similar to I38022 hypotheti | 8.86 | 3653 |
| 434096 | AW662958 | Hs.75825 | pleiomorphic adenoma gene-like 1 | 8.86 | 2815 |
| 407815 | AW373860 | Hs.183860 | hypothetical protein FLJ20277 | 8.76 | 201 |
| 412649 | NM_002206 | Hs.74369 | integrin, alpha 7 | 8.73 | 4884 654 |
| 424651 | AI493206 | Hs.32425 | ESTs | 8.68 | 1868 |
| 442560 | AA365042 | Hs.325531 | ESTs, Weakly similar to 2004399A chromos | 8.65 | 3441 |
| 407826 | AA128423 | Hs.40300 | calpain 3, (p94) | 8.63 | 202 |
| 442870 | N45018 | Hs.8769 | hypothetical protein DKFZp761J17121 | 8.58 | 3476 |
| 443062 | N77999 | Hs.8963 | Homo sapiens mRNA full length insert cDN | 8.57 | 3493 |
| 413856 | D13639 | Hs.75586 | cyclin D2 | 8.53 | 4907 758 |
| 422060 | R20893 | Hs.325823 | ESTs, Moderately similar to ALU5_HUMAN A | 8.51 | 1596 |
| 442572 | AI001922 | Hs.135121 | hypothetical protein FLJ22415 | 8.46 | 3445 |
| 430468 | NM_004673 | Hs.241519 | angiopoietin-like 1 | 8.42 | 2489 5452 |
| 417640 | D30857 | Hs.82353 | protein C receptor, endothelial (EPCR) | 8.38 | 1109 |
| 452165 | R17489 | Hs.28264 | Homo sapiens mRNA; cDNA DKFZp564L0822 (f | 8.36 | 4277 |
| 414061 | NM_000699 | Hs.335493 | amylase, alpha 2A; pancreatic | 8.34 | 4912 782 |
| 420139 | NM_005357 | Hs.95351 | lipase, hormone-sensitive | 8.34 | 1388 5065 |
| 418185 | AW958272 | Hs.347326 | intercellular adhesion molecule 2 (ICAM | 8.30 | 1168 |
| 422667 | H25642 | | ESTs | 8.29 | 1670 |
| 419047 | AW952771 | Hs.90043 | ESTs | 8.28 | 1269 |
| 417011 | F08212 | Hs.234898 | ESTs, Weakly similar to 2109260A B cell | 8.13 | 1051 |
| 451529 | AI917901 | Hs.208641 | ESTs | 8.13 | 4226 |
| 447540 | AL135716 | Hs.263780 | ESTs | 8.12 | 3876 |
| 449787 | AA005341 | | ESTs | 8.06 | 4082 |
| 409586 | AL050214 | Hs.55044 | DKFZP586H2123 protein | 8.02 | 385 4828 |
| 446161 | AA628206 | Hs.14125 | p53 regulated PA26 nuclear protein | 8.01 | 3729 |
| 435359 | T60843 | Hs.189679 | ESTs | 8.00 | 2909 |
| 413190 | AA151802 | Hs.40368 | adaptor-related protein complex 1, sigma | 8.00 | 698 |
| 428748 | AW593206 | Hs.98785 | Ksp37 protein | 7.99 | 2290 |
| 450787 | AB006190 | Hs.25475 | aquaporin 7 | 7.96 | 4164 5735 |
| 428957 | NM_003881 | Hs.194679 | WNT1 inducible signaling pathway protein | 7.95 | 2319 5384 |
| 427544 | AI767152 | Hs.181400 | ESTs, Weakly similar to I78885 serine/th | 7.88 | 2163 |
| 451583 | AI653797 | Hs.24133 | ESTs | 7.82 | 4230 |
| 408988 | AL119844 | Hs.49476 | Homo sapiens clone TUA8 Cri-du-chat regi | 7.81 | 320 |
| 414555 | N98569 | Hs.76422 | phospholipase A2, group IIA (platelets, | 7.81 | 844 |
| 438411 | H91928 | Hs.169370 | gb: ys81c10.r1 Soares retina N2b4HR Homo | 7.81 | 3137 |
| 439551 | W72062 | Hs.11112 | ESTs | 7.81 | 3214 |
| 415165 | AW887604 | Hs.78065 | complement component 7 | 7.81 | 913 |
| 408741 | M73720 | Hs.646 | carboxypeptidase A3 (mast cell) | 7.80 | 300 4805 |
| 422033 | AW245805 | Hs.110903 | claudin 5 (transmembrane protein deleted | 7.78 | 1594 |
| 439310 | AF086120 | Hs.102793 | ESTs | 7.69 | 3198 |
| 442831 | AI798959 | Hs.131686 | ESTs | 7.66 | 3473 |
| 429697 | AW296451 | Hs.24605 | ESTs | 7.66 | 2407 |
| 452390 | AI864142 | Hs.29288 | hypothetical protein FLJ21865 | 7.65 | 4303 |
| 421964 | X73079 | Hs.288579 | polymeric immunoglobulin receptor | 7.65 | 1586 5131 |
| 427164 | AB037721 | Hs.173871 | KIAA1300 protein | 7.65 | 2129 5332 |
| 424580 | AA446539 | Hs.339024 | ESTs, Weakly similar to A46010 X-linked | 7.65 | 1860 |
| 448710 | T62926 | Hs.304184 | ESTs | 7.63 | 3999 |
| 429580 | AA346839 | Hs.209100 | DKFZP434C171 protein | 7.58 | 2390 |
| 410611 | AW954134 | Hs.20924 | KIAA1628 protein | 7.58 | 497 |
| 451154 | AA015879 | Hs.33536 | ESTs | 7.58 | 4198 |
| 420311 | AW445044 | Hs.38207 | Human DNA sequence from clone RP4-530I15 | 7.57 | 1409 |
| 429640 | U83508 | Hs.2463 | angiopoietin 1 | 7.57 | 2400 5419 |
| 421124 | AI366452 | Hs.184430 | ESTs | 7.55 | 1483 |
| 409007 | AL122107 | Hs.49599 | Homo sapiens mRNA; cDNA DKFZp434G0827 (f | 7.52 | 322 |
| 411955 | X05153 | Hs.72938 | lactalbumin, alpha- | 7.49 | 4871 592 |
| 407828 | AW959500 | Hs.49597 | retinoic acid induced 2 | 7.49 | 203 |
| 417355 | D13168 | Hs.82002 | endothelin receptor type B | 7.48 | 1085 4981 |
| 429297 | X82494 | Hs.198862 | fibulin 2 | 7.45 | 2352 5399 |
| 426406 | AI742501 | Hs.169756 | complement component 1, s subcomponent | 7.45 | 2062 |
| 409472 | BE154093 | | gb: PM1-HT0340-091199-001-h02 HT0340 Homo | 7.42 | 378 |
| 449916 | T60525 | Hs.299221 | pyruvate dehydrogenase kinase, isoenzyme | 7.38 | 4090 |
| 433855 | AA834082 | Hs.307559 | ESTs | 7.37 | 2792 |
| 442915 | AA852875 | Hs.8850 | a disintegrin and metalloproteinase doma | 7.36 | 3480 |
| 409385 | AA071267 | | gb: zm61g01.r1 Stratagene fibroblast (937 | 7.31 | 365 |
| 451573 | AW130351 | | ESTs | 7.30 | 4229 |
| 444637 | T19101 | Hs.11494 | fibulin 5 | 7.29 | 3614 |
| 442070 | BE244622 | Hs.8084 | hypothetical protein dJ465N24.2.1 | 7.29 | 3394 |
| 458081 | AW014287 | Hs.41587 | RAD50 (S. cerevisiae) homolog | 7.28 | 4609 |
| 426992 | BE244961 | Hs.343200 | FE65-LIKE 2 | 7.26 | 2118 |
| 420570 | AI453665 | Hs.290870 | ESTs, Weakly similar to I38588 reverse t | 7.24 | 1434 |
| 401929 | | | C17001690: gi\|6005701\|ref\|NP_009099.1\|AT | 7.23 | |
| 449109 | AW270992 | Hs.120949 | ESTs, Weakly similar to ALU7_HUMAN ALU S | 7.21 | 4037 |
| 453510 | AI699482 | Hs.42151 | ESTs | 7.18 | 4415 |
| 405443 | | | Target Exon | 7.15 | |
| 432519 | AI221311 | Hs.130704 | ESTs, Weakly similar to BCHUIA S-100 pro | 7.14 | 2676 |
| 417054 | AF017060 | | aldehyde oxidase 1 | 7.13 | 1058 4977 |
| 459501 | AA854133 | Hs.310462 | ESTs | 7.10 | 4668 |
| 410057 | R66634 | Hs.268107 | multimerin | 7.09 | 436 |
| 425438 | T62216 | Hs.270840 | ESTs | 7.09 | 1963 |
| 447109 | X69086 | Hs.286161 | Homo sapiens cDNA FLJ13613 fis, clone PL | 7.07 | 3822 5681 |
| 444331 | AW193342 | Hs.24144 | ESTs | 7.04 | 3590 |
| 452040 | AW973242 | Hs.293690 | ESTs, Weakly similar to I38022 hypotheti | 7.04 | 4265 |
| 453870 | AW385001 | Hs.8042 | Homo sapiens cDNA: FLJ23173 fis, clone L | 7.02 | 4439 |
| 413497 | BE177661 | | gb: RC1-HT0598-020300-011-h02 HT0598 Homo | 7.01 | 729 |
| 431728 | NM_007351 | Hs.268107 | multimerin | 7.00 | 2593 5490 |
| 417780 | Z43482 | | collagen, type XI, alpha 1 | 7.00 | 1123 |
| 409060 | AI815867 | Hs.50130 | necdin (mouse) homolog | 6.99 | 328 |
| 423099 | NM_002837 | Hs.123641 | protein tyrosine phosphatase, receptor t | 6.98 | 1718 5168 |
| 419846 | NM_015977 | Hs.285681 | Williams-Beuren syndrome chromosome regi | 6.97 | |
| 414516 | AI307802 | | ESTs, Weakly similar to T43458 hypotheti | 6.97 | 838 |
| 425023 | AW956889 | Hs.154210 | EDG-1 (endothelial differentiation, sph | 6.93 | 1913 |
| 426822 | W78950 | Hs.220823 | ESTs | 6.92 | 2103 |
| 418283 | S79895 | Hs.83942 | cathepsin K (pycnodysostosis) | 6.91 | 1175 5007 |
| 435256 | AF193766 | Hs.13872 | cytokine-like protein C17 | 6.90 | 2903 5564 |
| 428722 | U76456 | Hs.190787 | tissue inhibitor of metalloproteinase 4 | 6.89 | 2286 5372 |
| 423007 | AA320134 | Hs.196029 | Homo sapiens mRNA for KIAA 1657 protein, | 6.88 | 1709 |
| 408048 | NM_007203 | Hs.42322 | A kinase (PRKA) anchor protein 2 | 6.87 | 228 4788 |
| 426128 | NM_001471 | Hs.167017 | gamma-aminobutyric acid (GABA) B recepto | 6.87 | |
| 427283 | AL119796 | Hs.174185 | ectonucleotide pyrophosphatase/phosphodi | 6.86 | 2137 |
| 426322 | J05068 | Hs.2012 | transcobalamin I (vitamin B12 binding pr | 6.85 | 2052 5299 |
| 429609 | AF002246 | Hs.210863 | cell adhesion molecule with homology to | 6.84 | 2394 5414 |
| 442662 | U78168 | Hs.8578 | Rap1 guanine-nucleotide-exchange factor | 6.82 | 3459 5624 |
| 408491 | AI088063 | Hs.7882 | ESTs | 6.79 | 272 |
| 451820 | AW058357 | Hs.199248 | ESTs | 6.77 | 4251 |
| 409549 | AB029015 | Hs.54886 | phospholipase C, epsilon 2 | 6.75 | 383 4827 |
| 421255 | BE326214 | Hs.93813 | ESTs | 6.73 | 1497 |
| 425869 | AA524547 | Hs.160318 | FXYD domain-containing ion transport reg | 6.72 | 2008 |
| 412567 | AI750979 | Hs.74034 | Homo sapiens clone 24651 mRNA sequence | 6.71 | 646 |
| 427019 | AA001732 | Hs.173233 | hypothetical protein FLJ10970 | 6.71 | 2122 |
| 443172 | AW662964 | Hs.199061 | p300/CBP-associated factor | 6.70 | 3502 |
| 414716 | AF199598 | Hs.97044 | Kv channel-interacting protein 2 | 6.70 | 4925 868 |
| 408731 | R85652 | | Homo sapiens mRNA; cDNA DKFZp434F1928 (f | 6.69 | 298 |
| 414522 | AW518944 | Hs.76325 | Immunoglobulin J chain | 6.67 | 840 |
| 412802 | U41518 | Hs.74602 | aquaporin 1 (channel-forming integral pr | 6.63 | 4887 671 |
| 425187 | AW014486 | Hs.22509 | ESTs | 6.61 | 1935 |
| 407938 | AA905097 | Hs.85050 | phospholamban | 6.60 | 216 |
| 443282 | T47764 | Hs.132917 | ESTs | 6.60 | 3517 |
| 444213 | T79623 | Hs.263351 | ESTs | 6.58 | 3584 |
| 407492 | S83198 | | gb: BPLP = basic proline-rich protein [huma | 6.57 | 165 4773 |
| 446714 | W73818 | Hs.110028 | ESTs | 6.57 | 3777 |
| 421853 | AL117472 | Hs.108924 | SH3-domain protein 5 (ponsin) | 6.55 | 1571 5125 |
| 417728 | AW138437 | Hs.24790 | KIAA1573 protein | 6.54 | 1117 |
| 452516 | AA058630 | Hs.29759 | RNA POLYMERASE I AND TRANSCRIPT RELEASE | 6.52 | 4320 |
| 406692 | L36607 | | gb: Homo sapiens (clone 22) pregnancy-spe | 6.51 | 4749 87 |
| 424252 | AK000520 | Hs.143811 | hypothetical protein FLJ20513 | 6.49 | 1821 5206 |
| 420832 | Z26248 | Hs.99962 | proteoglycan 2, bone marrow (natural kil | 6.48 | 1458 5089 |
| 414665 | AA160873 | | serum amyloid A1 | 6.48 | 859 |
| 443980 | AI459140 | Hs.109150 | ESTs | 6.47 | 3565 |
| 414742 | AW370946 | Hs.23457 | ESTs | 6.47 | 872 |
| 418728 | AW970937 | Hs.293843 | ESTs | 6.46 | 1232 |
| 419195 | AW291165 | Hs.25447 | ESTs | 6.45 | 1287 |
| 453880 | AI803166 | Hs.135121 | ESTs, Weakly similar to I38022 hypotheti | 6.43 | 4443 |
| 444561 | NM_004469 | Hs.11392 | c-fos induced growth factor (vascular en | 6.41 | 3606 5641 |
| 433793 | AW975959 | Hs.107513 | ESTs, Moderately similar to KIAA1058 pro | 6.41 | 2788 |
| 427809 | M26380 | Hs.180878 | lipoprotein lipase | 6.38 | 2196 |
| 443627 | AW138605 | Hs.134198 | ESTs | 6.36 | 3534 |
| 428411 | AW291464 | Hs.10338 | ESTs | 6.35 | 2251 |
| 421368 | L13283 | Hs.103944 | Homo sapiens (clone MG2-5-12) mucin (MG2 | 6.34 | 1514 |
| 430234 | N29317 | | KIAA1238 protein | 6.34 | 2457 |
| 423201 | NM_000163 | Hs.125180 | growth hormone receptor | 6.34 | 1728 5175 |
| 432030 | AI908400 | Hs.143789 | ESTs | 6.33 | 2628 |
| 422722 | H74219 | Hs.269772 | ESTs | 6.33 | 1675 |
| 411764 | T40064 | Hs.71968 | Homo sapiens mRNA; cDNA DKFZp564F053 (fr | 6.31 | 575 |
| 438414 | AA806794 | Hs.131511 | ESTs | 6.29 | 3138 |
| 446939 | AL133353 | Hs.16606 | CGI-32 protein | 6.28 | 3804 |
| 421465 | AK001020 | Hs.104627 | Homo sapiens cDNA FLJ10158 fis, clone HE | 6.28 | 1523 |
| 453467 | AI535997 | Hs.30089 | ESTs | 6.27 | 4410 |
| 435545 | AA687415 | Hs.28107 | ESTs | 6.25 | 2926 |
| 407172 | T54095 | | gb: ya92c05.s1 Stratagene placenta (93722 | 6.24 | 132 |
| 448406 | AW772298 | Hs.21103 | Homo sapiens mRNA; cDNA DKFZp564B076 (fr | 6.23 | 3964 |
| 417788 | AI436699 | Hs.84928 | nuclear transcription factor Y, beta | 6.22 | 1124 |
| 418452 | BE379749 | Hs.85201 | C-type (calcium dependent, carbohydrate- | 6.21 | 1202 |
| 431177 | NM_003304 | Hs.250687 | transient receptor potential channel 1 | 6.20 | 2536 5471 |
| 429331 | H13881 | Hs.143825 | ESTs | 6.20 | 2354 |
| 419925 | AA159850 | Hs.93765 | lipoma HMGIC fusion partner | 6.19 | 1370 |
| 453125 | AW779544 | Hs.115497 | hypothetical protein FLJ22655 | 6.19 | 4376 |
| 438303 | AB028998 | Hs.6147 | KIAA1075 protein | 6.15 | 3130 5593 |
| 427620 | NM_003705 | Hs.179866 | solute carrier family 25 (mitochondrial | 6.14 | 2172 5341 |
| 412568 | AI878826 | Hs.74034 | caveolin 1, caveolae protein, 22 kD | 6.06 | 647 |
| 435487 | W07343 | Hs.182538 | phospholipid scramblase 4 | 6.05 | 2919 |
| 420103 | AA382259 | Hs.95197 | aldehyde dehydrogenase 1 family, member | 6.02 | 1384 |
| 418336 | BE179882 | | glutathione peroxidase 3 (plasma) | 6.02 | 1188 |
| 448782 | AL050295 | | G-protein coupled receptor 116 (GPR116) | 6.01 | 4006 5713 |
| 407939 | W05608 | Hs.312679 | ESTs, Weakly similar to A49019 dynein he | 5.99 | 217 |
| 430714 | AA484757 | Hs.287601 | Homo sapiens cDNA FLJ13830 fis, clone TH | 5.99 | 2509 |
| 413630 | AL036883 | Hs.75450 | delta sleep inducing peptide, immunoreac | 5.98 | 742 |
| 417280 | AW173116 | Hs.250103 | ESTs | 5.98 | 1076 |
| 424880 | NM_000328 | Hs.153614 | retinitis pigmentosa GTPase regulator | 5.97 | 1895 5236 |
| 413200 | AA127395 | Hs.222414 | ESTs | 5.97 | 700 |
| 431704 | NM_006680 | Hs.2838 | malic enzyme 3, NADP()-dependent, mitoch | 5.96 | 2586 5487 |
| 447770 | AB032417 | Hs.19545 | frizzled (Drosophila) homolog 4 | 5.96 | 3904 5701 |
| 421237 | U25029 | Hs.102761 | Human glucocorticoid receptor alpha mRNA | 5.95 | 1493 5099 |
| 449539 | W80363 | Hs.58446 | ESTs | 5.95 | 4065 |
| 451108 | AW268884 | Hs.204387 | ESTs | 5.94 | 4194 |
| 430520 | NM_016190 | Hs.242057 | chromosome 1 open reading frame 10 | 5.94 | 2492 5454 |
| 419290 | AI128114 | Hs.112885 | spinal cord-derived growth factor-B | 5.93 | 1298 |
| 445234 | AW137636 | Hs.146059 | ESTs | 5.92 | 3860 |
| 446874 | AW968304 | Hs.56156 | ESTs | 5.92 | 3798 |
| 422994 | AW891802 | Hs.296276 | ESTs | 5.89 | 1707 |
| 425009 | X58288 | Hs.154151 | protein tyrosine phosphatase, receptor t | 5.89 | 1911 5243 |
| 422109 | S73265 | Hs.1473 | gastrin-releasing peptide | 5.88 | 1604 5138 |
| 416336 | R97949 | Hs.24128 | ESTs | 5.87 | 987 |
| 441944 | AW855861 | Hs.8025 | Homo sapiens clone 23767 and 23782 mRNA | 5.86 | 3381 |
| 447384 | AI377221 | Hs.40528 | ESTs | 5.86 | 3853 |
| 407978 | AW385129 | Hs.41717 | phosphodiesterase 1A, calmodulin-depende | 5.86 | 220 |
| 454338 | AW381251 | | gb: RC0-HT0297-301099-011-a08 HT0297 Homo | 5.85 | 4477 |
| 435266 | AK001942 | Hs.4863 | hypothetical protein DKFZp566A1524 | 5.85 | 2904 |
| 447571 | AF274863 | Hs.18889 | DKFZP434M183 protein | 5.85 | 3880 5693 |
| 432608 | AI492660 | Hs.170935 | ESTs | 5.83 | 2684 |
| 412420 | AL035668 | Hs.73853 | bone morphogenetic protein 2 | 5.79 | 4878 629 |
| 428809 | W56487 | Hs.193784 | Homo sapiens mRNA; cDNA DKFZp586K1922 (f | 5.77 | 2302 |
| 412622 | AW664708 | Hs.171959 | ESTs | 5.75 | 651 |
| 439177 | AW820275 | Hs.76611 | ESTs, Weakly similar to I38022 hypotheti | 5.75 | 3189 |
| 450954 | AI904740 | Hs.25691 | receptor (calcitonin) activity modifying | 5.74 | 4178 |
| 416889 | AW250318 | Hs.80395 | mal, T-cell differentiation protein | 5.74 | 1034 |
| 418965 | AI002238 | Hs.11482 | splicing factor, arginine/serine-rich 11 | 5.74 | 1264 |
| 436473 | AI193122 | Hs.132275 | ESTs | 5.73 | 2987 |
| 430733 | AW975920 | Hs.121036 | ESTs | 5.73 | 2510 |
| 426628 | AW901932 | Hs.278582 | v-akt murine thymoma viral oncogene Homo | 5.70 | 2084 |
| 447894 | AW204253 | Hs.21912 | ESTs | 5.70 | 3919 |
| 424585 | AA464840 | Hs.131987 | ESTs | 5.68 | 1862 |
| 410485 | AW750242 | | gb: RC1-BT0567-301299-011-e02 BT0567 Homo | 5.66 | 483 |
| 439484 | AW970218 | Hs.135254 | Homo sapiens clone 1 thrombospondin mRNA | 5.64 | 3211 |
| 459247 | N46243 | Hs.110373 | ESTs, Highly similar to T42626 secreted | 5.64 | 4662 |
| 416749 | AW068550 | Hs.79732 | fibulin 1 | 5.64 | 1020 |
| 452093 | AA447453 | Hs.27860 | Homo sapiens mRNA; cDNA DKFZp586M0723 (f | 5.62 | 4269 |
| 443510 | NM_012190 | Hs.9520 | formyltetrahydrofolate dehydrogenase | 5.62 | 3526 5628 |
| 407374 | AA724738 | Hs.131034 | ESTs, Weakly similar to I78885 serine/th | 5.62 | 157 |
| 417317 | AW296584 | Hs.293782 | ESTs | 5.62 | 1080 |
| 407960 | F34014 | Hs.62914 | ESTs | 5.61 | 219 |
| 420733 | AW291446 | Hs.88651 | ESTs | 5.61 | 1446 |
| 443605 | H06865 | Hs.134131 | ESTs | 5.59 | 3531 |
| 406694 | M94891 | Hs.225932 | pregnancy specific beta-1-glycoprotein 7 | 5.57 | 4750 88 |
| 421177 | AW070211 | Hs.102415 | Homo sapiens mRNA; cDNA DKFZp586N0121 (f | 5.56 | 1485 |
| 427136 | AL117415 | Hs.173716 | a disintegrin and metalloproteinase doma | 5.55 | 2126 |
| 431981 | AA664069 | Hs.115779 | ESTs | 5.55 | 2623 |
| 421712 | AK000140 | Hs.107139 | hypothetical protein | 5.53 | 1556 |
| 426485 | NM_006207 | Hs.170040 | platelet-derived growth factor receptor- | 5.51 | 2069 5306 |
| 419150 | T29618 | Hs.89640 | TEK tyrosine kinase, endothelial (venous | 5.51 | 1282 |
| 420517 | AB011115 | Hs.98507 | KIAA0543 protein | 5.50 | 1425 5078 |
| 450253 | AL133047 | Hs.24715 | Homo sapiens mRNA; cDNA DKFZp434D0215 (f | 5.49 | 4119 5727 |
| 402575 | | | Rho GTPase activating protein 1 | 5.49 | |
| 447974 | R76886 | | gb: yi64b03.s1 Soares placenta Nb2HP Homo | 5.49 | 3930 |
| 429610 | AB024937 | Hs.211092 | LUNX protein; PLUNC (palate lung and nas | 5.48 | 2395 5415 |
| 424789 | BE176694 | Hs.279860 | tumor protein, translationally-controlle | 5.48 | 1886 |
| 428695 | AI355647 | Hs.189999 | purinergic receptor (family A group 5) | 5.46 | 2282 |
| 436805 | AA731533 | Hs.270751 | ESTs | 5.46 | 3008 |
| 459513 | AI032946 | | gb: ox06g09.s1 Soares_fetal_liver_spleen_{—} | 5.45 | 4671 |
| 418481 | M81945 | | CD34 antigen | 5.44 | 1206 5018 |
| 414121 | AA151719 | Hs.95834 | ESTs | 5.44 | 790 |
| 410544 | AI446543 | Hs.95511 | ESTs | 5.44 | 489 |
| 407221 | U25987 | | pregnancy specific beta-1-glycoprotein 9 | 5.43 | 138 4765 |
| 436062 | AK000027 | Hs.98633 | ESTs | 5.43 | 2962 |
| 417696 | BE241624 | Hs.82401 | CD69 antigen (p60, early T-cell activati | 5.43 | 1113 |
| 434230 | AA551569 | | hypothetical protein PRO2822 | 5.42 | 2823 |
| 421429 | NM_014922 | Hs.104305 | death effector filament-forming Ced-4-li | 5.41 | 1517 5106 |
| 438797 | C16161 | Hs.283040 | hypothetical protein PRO2543 | 5.40 | 3166 |
| 454043 | AW451951 | Hs.29041 | Homo sapiens cDNA FLJ14177 fis, clone NT | 5.38 | 4464 |
| 428531 | AW274831 | | ESTs | 5.37 | 2267 |
| 437891 | AW006969 | Hs.6311 | hypothetical protein FLJ20859 | 5.35 | 3103 |
| 442713 | D63203 | Hs.184627 | KIAA0118 protein | 5.35 | 3462 |
| 449282 | AL048056 | Hs.23437 | Homo sapiens cDNA FLJ13555 fis, clone PL | 5.34 | 4054 |
| 407247 | S83198 | Hs.87198 | basic proline-rich protein | 5.34 | 145 4769 |
| 414831 | M31158 | Hs.77439 | protein kinase, cAMP-dependent, regulato | 5.34 | 4931 883 |
| 410116 | AW630671 | Hs.58636 | squamous cell carcinoma antigen recogniz | 5.33 | 447 |
| 426354 | NM_004010 | Hs.169470 | dystrophin (muscular dystrophy, Duchenne | 5.33 | 2057 |
| 430265 | L36033 | Hs.237356 | stromal cell-derived factor 1 | 5.33 | 2458 5439 |
| 433688 | AA628467 | Hs.112572 | Homo sapiens cDNA FLJ14130 fis, clone MA | 5.33 | 2780 |
| 428939 | AW236550 | Hs.131914 | ESTs | 5.32 | 2318 |
| 427605 | NM_000997 | Hs.337445 | ribosomal protein L37 | 5.31 | 2171 5340 |
| 433138 | AB029496 | Hs.59729 | semaphorin sem2 | 5.30 | 2737 5528 |
| 441319 | AI354869 | Hs.133081 | ESTs, Weakly similar to T08700 hypotheti | 5.30 | 3346 |
| 450389 | AW014016 | | ESTs | 5.29 | 4136 |
| 406686 | M37755 | | gb: Human pregnancy-specific beta-1-glyco | 5.29 | 4746 84 |
| 428336 | AA503115 | Hs.183752 | microseminoprotein, beta- | 5.29 | 2242 |
| 442287 | AW952703 | Hs.8182 | synaptic nuclei expressed gene 1b | 5.28 | 3411 |
| 422431 | AI769410 | Hs.221461 | ESTs | 5.28 | 1646 |
| 423023 | N50128 | Hs.173400 | ESTs | 5.25 | 1712 |
| 413802 | AW964490 | Hs.32241 | ESTs, Weakly similar to S65657 alpha-1C- | 5.25 | 754 |
| 446584 | U53445 | Hs.15432 | downregulated in ovarian cancer 1 | 5.25 | 3766 5673 |
| 432128 | AA127221 | Hs.296502 | Interleukin 1 receptor-like 1 | 5.25 | 2633 |
| 411151 | AW866497 | | gb: QV4-SN0024-170400-176-e07 SN0024 Homo | 5.24 | 538 |
| 412330 | NM_005100 | Hs.788 | A kinase (PRKA) anchor protein (gravin) | 5.24 | 4877 625 |
| 452422 | AA521416 | | tumor necrosis factor receptor superfami | 5.24 | 4305 |
| 424244 | AV647184 | Hs.143601 | hypothetical protein hCLA-iso | 5.24 | 1819 |
| 428280 | H05541 | Hs.183428 | sarcospan (Kras oncogene-associated gene | 5.23 | 2235 |
| 451683 | AI808964 | Hs.207673 | ESTs | 5.23 | 4239 |
| 441723 | R72137 | Hs.7949 | DKFZP586B2420 protein | 5.22 | 3370 |
| 408000 | L11690 | Hs.198689 | bullous pemphigoid antigen 1 (230/240 kD) | 5.22 | 223 4786 |
| 439091 | AA830144 | Hs.135613 | ESTs, Moderately similar to I38022 hypot | 5.21 | 3184 |
| 439411 | AA044876 | Hs.58043 | ESTs, Weakly similar to CYA2_HUMAN ADENY | 5.20 | 3206 |
| 419917 | AA320068 | Hs.93701 | Homo sapiens mRNA; cDNA DKFZp434E232 (fr | 5.20 | 1369 |
| 458616 | AV656098 | Hs.172382 | betaine-homocysteine methyltransferase 2 | 5.19 | 4629 |
| 444523 | AW409958 | Hs.323396 | hypothetical protein RP1-317E23 | 5.19 | 3604 |
| 406714 | AI219304 | Hs.266959 | hemoglobin, gamma G | 5.19 | 92 |
| 437140 | AA312799 | Hs.283689 | activator of CREM in testis | 5.19 | 3035 |
| 427890 | AA435761 | | ESTs | 5.18 | 2203 |
| 408776 | AA057365 | | ESTs, Weakly similar to I38022 hypotheti | 5.18 | 306 |
| 407571 | AI446183 | Hs.9572 | ESTs, Highly similar to CYA5_HUMAN ADENY | 5.17 | 170 |
| 407183 | AA358015 | | gb: EST66864 Fetal lung III Homo sapiens | 5.16 | 134 |
| 414005 | AA134489 | | ESTs | 5.16 | 773 |
| 449679 | AI823951 | Hs.129700 | tolloid-like 1 | 5.15 | 4073 |
| 436306 | AA805939 | Hs.117927 | ESTs | 5.15 | 2978 |
| 408221 | AA912183 | Hs.47447 | ESTs | 5.14 | 246 |
| 412668 | AA456195 | | hypothetical protein FLJ14621 | 5.14 | 658 |
| 443547 | AW271273 | | hypothetical protein FLJ12666 | 5.14 | 3529 |
| 449365 | AW968261 | Hs.118913 | ESTs, Moderately similar to T46371 hypot | 5.12 | 4058 |
| 433165 | AA578904 | Hs.292437 | ESTs | 5.12 | 2743 |
| 417067 | AJ001417 | Hs.81086 | solute carrier family 22 (extraneuronal | 5.12 | 1060 4978 |
| 437948 | AA772920 | Hs.303527 | ESTs | 5.12 | 3110 |
| 447391 | AI377444 | Hs.54245 | ESTs, Weakly similar to S65824 reverse t | 5.11 | 3855 |
| 437652 | AF036144 | Hs.5734 | meningioma expressed antigen 5 (hyaluron | 5.11 | 3078 5588 |
| 434633 | AI189587 | Hs.120915 | ESTs | 5.10 | 2855 |
| 421341 | AJ243212 | | deleted in malignant brain tumors 1 | 5.10 | 1509 5102 |
| 420908 | AL049974 | Hs.100261 | Homo sapiens mRNA; cDNA DKFZp564B222 (fr | 5.10 | 1462 |
| 410526 | R05652 | Hs.64125 | Homo sapiens clone 24617 mRNA sequence | 5.09 | 488 |
| 412460 | AW953287 | Hs.83071 | ESTs | 5.09 | 635 |
| 451245 | AA016218 | Hs.58231 | ESTs | 5.08 | 4203 |
| 444880 | AW118683 | Hs.154150 | ESTs | 5.08 | 3636 |
| 448425 | AI500359 | | ESTs | 5.07 | 3968 |
| 421932 | W51778 | Hs.323949 | kangai 1 (suppression of tumorigenicity | 5.06 | 1581 |
| 433854 | AA610649 | Hs.333239 | ESTs | 5.06 | 2791 |
| 451742 | T77609 | | ankyrin 2, neuronal | 5.05 | 4245 |
| 431176 | AI026984 | Hs.293662 | ESTs | 5.05 | 2535 |
| 445399 | AI681906 | Hs.4786 | Homo sapiens cDNA: FLJ22849 fis, clone K | 5.05 | 3674 |
| 437027 | A8023235 | Hs.5400 | KIAA1018 protein | 5.05 | 3030 5583 |
| 432527 | AW975028 | Hs.102754 | ESTs | 5.04 | 2677 |
| 433740 | AA934994 | Hs.112628 | ESTs | 5.03 | 2785 |
| 431638 | NM_000916 | Hs.2820 | oxytocin receptor | 5.02 | 2579 5484 |
| 436037 | AA917639 | Hs.13063 | ESTs | 5.02 | 2958 |
| 423488 | AW748714 | Hs.194720 | gb: QV3-BT0294-241199-025-c01 BT0294 Homo | 5.02 | 1751 |
| 426464 | NM_004334 | Hs.169998 | bone marrow stromal cell antigen 1 | 5.02 | 2067 5304 |
| 450697 | AW152166 | Hs.182113 | ESTs | 5.01 | 4151 |
| 436169 | AA888311 | Hs.17602 | Homo sapiens cDNA FLJ12381 fis, clone MA | 5.01 | 2966 |
| 453143 | AA382234 | | protein tyrosine phosphatase, receptor t | 5.01 | 4378 |
| 414545 | AA149287 | Hs.76605 | ESTs | 5.00 | 843 |
| 421126 | M74587 | Hs.102122 | insulin-like growth factor binding prote | 4.99 | 1484 5095 |
| 413401 | AI361861 | Hs.118659 | ESTs | 4.98 | 714 |
| 436919 | AW297307 | Hs.204562 | ESTs | 4.97 | 3021 |
| 453828 | AW970960 | Hs.293821 | ESTs | 4.96 | 4433 |
| 418318 | U47732 | Hs.84072 | transmembrane 4 superfamily member 3 | 4.95 | 1182 |
| 408669 | AI493591 | Hs.78146 | platelet/endothelial cell adhesion molec | 4.94 | 290 |
| 414919 | AW087337 | Hs.194461 | ESTs | 4.94 | 893 |
| 438608 | AI380529 | Hs.44628 | ESTs | 4.94 | 3152 |
| 414138 | AA135884 | Hs.3772 | ESTs | 4.94 | 791 |
| 434365 | AI073378 | Hs.126793 | ESTs | 4.94 | 2834 |
| 408242 | AA251594 | Hs.43913 | PIBF1 gene product | 4.94 | 247 |
| 437773 | U24186 | Hs.283018 | replication protein A complex 34 kd subu | 4.93 | 3090 5592 |
| 445529 | H14421 | Hs.180513 | ATP-binding cassette, sub-family A (ABC1 | 4.93 | 3683 |
| 434389 | AA971235 | Hs.128098 | ESTs | 4.90 | 2837 |
| 407758 | D50915 | Hs.38365 | KIAA0125 gene product | 4.90 | 192 4782 |
| 424776 | AI867931 | Hs.164595 | ESTs | 4.89 | 1885 |
| 448122 | AW665656 | Hs.173187 | ESTs | 4.88 | 3946 |

Pkey: Unique Eos probeset identifier number
ExAccn: Exemplar Accession number, Genbank accession number
UniGeneID: UniGene number
UniGene Title: UniGene gene title
R1: 90th percentile of breast metastases to the brain Als divided by the 90th percentile of normal breast tissue Als, where the 15th percentile of all normal body tissue Als was subtracted from the both numerator and denominator.
SEQ ID NO(s): SEQ ID number(s) for nucleic acid and protein sequences associated with table entry.

TABLE 5B

| Pkey | CAT number | Accession |
|---|---|---|
| 421998 | 133592_1 | B1757233 BG911321 BF351759 AW244016 AW026834 AW024260 AI420138 AA779354 AI093360 AI934858 AW151292 AI373133 AI335587 |
| | | AI969728 AAI01632 BE218525 AI802114 AI783721 AA845265 AW088826 AI832852 F03967 AI611148 AI720358 AW293764 N91161 |
| | | R79192 W85852 AW771263 BG820263 BG012864 R74441 R86080 W04256 BE707244 BF899452 BE327552 BE669500 AI492388 |
| | | AI241532 BF448184 AI209012 AA886528 N70309 AW582776 BF110563 BF448329 BE326537 AW770471 BF444926 BE674147 AI793266 |
| | | AI991774 AI807726 AI218667 AA301750 R44328 |
| 411962 | 2307710_1 | AA099050 AA099526 T47733 |
| 414496 | 1526_1 | AK058006 BF724822 W65303 AW887764 AW023806 Z25353 AW022095 AA730973 W00417 W73819 BF982096 AI927669 AW188021 |
| | | AW770478 AI913512 AA604358 AI697341 AI691028 AI338392 AI079403 H97538 AI144448 AI253102 AI051402 AI335900 AI868132 |
| | | N28900 H98465 AF268386 AI799915 AI819228 BE048413 AW304723 AI819923 BF223106 AA155907 AW298079 BF055272 BF446804 |
| | | BF197697 W58588 BF197538 AA032180 AA992597 AW590254 AA027824 AI129369 AI131331 AI655843 AA932907 AW104493 AI150615 |
| | | BF110226 AW172271 AI312659 AA057312 BE673669 AA722984 AW104985 AI129232 AI078648 AI653086 AI703481 AW515897 AI352206 |
| | | N67076 AW297281 AI686162 AA029184 AI610743 AW772016 AI091778 W65401 AI687374 AI218085 AI765158 AI018002 AI653068 |
| | | AI335704 AI520850 AW275228 AW275204 AI420247 AA975336 AI697042 AW182235 AA736386 AI281682 AW169698 AW263325 |
| | | BE645834 AI377438 AI146706 AA613808 AA716538 B1496247 AA032248 AI698930 AI193399 N70026 H86792 AA404489 W61267 |
| | | BF447230 AA910805 AA150774 AA621907 AA902526 AI827634 AW022037 BF059000 BI496246 BG577007 BG571077 AA460779 |
| | | AW816890 AW816893 AW816891 AA029183 AA010295 H86850 T83320 BE160823 H12925 N40087 AA096372 BE160847 AW816892 |
| | | AW816889 AW816882 AW816868 AW816941 AW816578 AW816940 AWB16577 AI431628 AI828113 AA033677 AA033654 AA452704 |
| | | AA317582 AA346971 BF836584 H48669 B1861605 BG925200 AA463277 H89048 AAI55952 W03252 W01510 W00915 W58589 AA164519 |
| | | N24017 N24622 N27149 N70109 R43771 AA010296 W84611 H98889 H88965 AW594424 AA034139 AA065223 N99696 BG981481 N94371 |
| | | AA767970 W47146 N70977 H05510 W61268 T90796 AA164518 W47244 AA150883 AA034138 BF338483 F13671 H51317 W72716 N21488 AI188071 AI370541 AI754442 |
| | | AA148524 AI749182 W95221 W92522 R20385 |
| 407102 | 7177_2 | AW945170 BF930905 F33652 BG057818 AI368018 AI421485 AI300352 AI378525 AI264177 AI276281 AI245302 AI281050 AI190036 |
| | | AW451438 AW242903 AA910870 F22289 F19647 F22375 AW473816 BF445785 AA774528 F33447 C01077 AW772227 F17759 H42812 |
| | | R09701 AA349096 R48772 H42892 H42537 R47898 N28263 H25721 F32386 H43971 R48205 F21390 H45809 AA007629 R47897 R83734 |
| | | H45844 AW983653 H43970 H42536 H24495 R48875 H42961 H22079 R86018 |
| 406800 | 0_0 | AA505535 |
| 412295 | 133592_1 | B1757233 BG911321 BF351759 AW244016 AW026834 AW024260 AI420138 AA779354 AI093360 AI934858 AW151292 AI373133 AI335587 |
| | | AI969728 AA101632 BE218525 AI802114 AI783721 AA845265 AW088826 AI832852 F03967 AI611148 AI720358 AW293764 N91161 |
| | | R79192 W85852 AW771263 BG820263 BG012864 R74441 R86080 W04256 BE707244 BF899452 BE327552 BE669500 AI492388 |
| | | AI241532 BF448184 AI209012 AA886528 N70309 AW582776 BF110563 BF448329 BE326537 AW770471 BF444926 BE674147 AI793266 |
| | | AI991774 AI807726 AI218667 AA301750 R44328 |
| 408503 | 24663_2 | NM_054110 BC014789 BI552129 BG896227 BG900557 AY035399 AW119059 BF432376 AW629242 AA971319 AI378176 AI275884 |
| | | AA236602 BG898257 BG897636 BG900588 AF361251 BG926353 BG714178 AA368180 BG896473 BG715977 BG900230 BG901163 |
| | | BF342939 |
| 444567 | 42058_1 | BE048349 AW974054 R79064 AA854763 AA780852 AW129141 AI061419 AW469162 AA602558 AA975901 H65073 H94455 AA969673 |
| | | BE048171 R63499 R92783 N68673 R62964 R31280 AA873363 R92768 AI740671 N41012 H72843 N54939 N91687 H58308 N53116 |
| | | AV650793 AV650271 BI489336 R28494 |
| 422667 | 224778_1 | AI758223 AW469334 BF940841 AW080348 AI270363 AI055892 BE464168 BF431797 BE350144 BF448739 AI693409 BF432999 D62848 |
| | | AA398070 AI383375 AW611490 |
| 449787 | 79759_1 | AW975633 AW961632 AA322539 AW513289 AI864190 AW073506 AI016894 AA747635 AA730405 AA418033 AI609549 AI873981 |
| | | AA005341 BE182658 AA004291 |
| 409472 | 813633_1 | BE162685 BE154093 BE154085 |
| 409385 | 110758_1 | T65940 T64515 AA071267 AA071334 |
| 451573 | 625119_1 | AW338699 AI803973 AW130351 |
| 417054 | 12405_2 | BG533564 BG618564 AW296119 AI269233 BF508328 AW364777 AW292258 AA371049 AI452471 AI092522 BG618376 AL049080 |
| | | AA631068 BG564643 T53833 AV702544 BG533452 AV705004 AA588281 T28665 BG569026 AV646874 AV647253 AV647455 AV647749 |
| | | BI759444 AV652457 AV695354 AV696010 AV697248 BG617586 AV722549 AI435836 AI590676 AI245019 AW338243 AA530898 D52191 |
| | | AI435352 D57473 BG566952 AI420505 AA035245 AV704972 BG564113 AI439237 AI287456 AV695686 AA349017 |
| 413497 | 1518002_1 | BE177661 H06215 BE144709 BE144829 |
| 417780 | 2147534_1 | R14659 Z43482 R52906 |
| 414516 | 60847_1 | AK057782 AI146454 BG703115 AI765980 AI948611 AA889263 AA947457 BG547193 H11947 Z38147 F10426 BF447329 H11946 T74968 |
| | | F06195 BG548563 AI004988 AAI48735 AI307802 AI439791 BE041453 AI984904 AA148734 F12823 |
| 408731 | 11725_2 | AF305826 BE350971 AI765355 AW172600 AI310231 BM271766 AI547292 AW612019 AI674617 AW138666 AI147629 AI147620 BF857810 |
| | | BF886300 BF885952 BF886303 BF885956 BG565497 BE670834 AA114025 BF886396 BF886928 AV750861 D62864 |
| 407492 | 65295_1 | S83198 NM_021225 BG212392 BG218751 BG191209 BG212926 BG185436 |
| 406692 | 0_0 | L36607 |
| 414665 | 23751_3 | BG567713 AW665841 AI814924 AW978339 AW264036 AI373950 AW183157 AW082249 AI201658 AI364196 AA150743 AA160873 |
| | | AA453757 BF871646 BF871640 AA565311 AA989511 R10152 AA807154 T77900 AW51591 |
| 430234 | 1746_1 | AW025803 AL137567 AI760919 BE552289 AW082686 AA913951 BE501313 AV756373 BE048863 AA828185 BG151502 AI655583 |
| | | AW473377 AI949888 AA453495 AW130287 AI222766 AI862122 AI309288 AL038626 AA714749 AI719007 W95486 N29317 AW589706 |
| | | AA905486 AI744057 AA759318 AI084950 AI273294 BF835579 AW235310 AI914478 BF887920 R21884 AI479541 AW189671 AW235752 |
| | | AI762157 BG698714 AA461269 AA460813 W95524 AA301369 AI760649 BF854009 AW303856 H42831 AV37305 H03702 R22396 R65961 |
| 407172 | 452462_1 | BG925072 BG925045 AV738744 T54095 T54186 BI000208 BI003704 BI002312 BG990736 BI000200 BI004083 BI004085 BI000196 |
| | | BG990731 BG990735 |
| 418336 | 58817_1 | AW969583 BI772505 BE179578 AI493714 AI937718 AA663709 BI868925 AW138743 AI911314 BE645538 BG911947 AI380325 AI265803 |
| | | W56175 AI658779 AI675997 AW665991 AI459263 AI420121 Z38874 AA570115 AW301008 AA216257 BF062662 BI772789 H05989 |
| | | BF085523 BI001277 |
| 448782 | 34980_1 | AB018301 AL050295 BF513128 AW385080 AL551708 AI352542 AI829703 AI819388 AW629019 AW073189 AW273857 AW118768 |
| | | AI453845 AI452494 AA886341 AI057144 AA904647 AI423547 AW263913 AI094774 AI434419 AI039546 AI002491 AI240412 Z25099 |
| | | AA995178 AW050649 AW026140 AI796309 AI584012 BE166666 AI767991 AI309041 AA724059 AI695284 AI245095 T63971 Z40627 |
| | | BE166681 BG570071 BF921915 BI562702 BG506502 AV658066 R48378 AA121543 AI096938 AA618131 H40993 R48277 AI352281 |
| | | BG540263 BG538901 N95226 AI356752 AI221152 Z28777 R16574 AW966449 AA044116 AW797518 BI010405 AA044288 AI093508 BE140169 T64039 BG433106 AW130367 |
| | | AW130361 N73937 AA127680 AW044037 AI096437 AA384077 BF941499 T93764 BG003285 |
| 454338 | 788429_2 | AW381251 |
| 410485 | 1028443_1 | AW750242 T68507 |
| 447974 | 1092239_1 | BG944967 AI453674 R77049 R76886 |
| 459513 | 417837_1 | AV704062 BE162284 AI032946 BF360636 |
| 418481 | 17381_1 | S53911 NM_001773 AL572644 AL550179 AI688653 AW025002 AW614285 AU158779 AI017002 AA434387 AI252665 AI262206 AU147582 |
| | | AI144193 AW952860 AI128776 AI017793 AI160509 AA906021 AI149563 AU154950 AI128488 W74409 AI970362 AI141453 AU158772 |
| | | W58493 BM148338 R73091 AW020496 BI491517 AA022917 AI432610 AI879448 R69099 AI708954 AW014274 AA483672 AA528783 |
| | | AA912271 AI926942 BE677587 AI874217 AA152376 AA640408 AI287334 BF830285 AA311473 C18678 AA922603 C00910 AI124073 |
| | | R38730 AA043439 W94644 W58646 AA664247 BE061934 H01096 R69613 AI383162 AU133723 AA311526 R67942 H01097 H72113 |
| | | R72430 R39494 AV744074 AA535925 BI759288 BI052385 BF854687 AW608286 AA043438 R72478 AL513811 R69214 AA188435 |
| | | AA054965 |
| 434230 | 41110_1 | AF119900 NM_018539 AA702388 N53043 BF351064 N70103 AI207469 AA551569 AW383189 W00906 W00935 N54252 |
| 428531 | 715072_1 | AI672553 AW274831 AA429897 AA446046 AA860950 |
| 450389 | 2018_25 | AI694475 AW341358 AW014016 |
| 411151 | 1071416_1 | AW819775 AW866497 AW819868 AW866602 AW866561 |
| 452422 | 10123_3 | AK056500 AL520733 AA521416 AW967877 AI799849 AA470754 AI674241 AI343684 BF589270 AA026154 AI473803 AW612523 AI344466 |
| | | AI344464 AI216171 AA055926 AA255890 BF967590 AA055329 AA026153 W52679 AA135902 AI721140 C14114 R43875 AI761376 R58888 |
| | | AA135901 BF849170 AA725347 AI908777 AA719606 |
| 427890 | 1373988_1 | AA417099 AA435761 AA972917 AI660387 |
| 408776 | 106302_1 | BF057799 BE218747 T15720 AA057365 AI811370 |
| 414005 | 259333_1 | AW968220 AA259126 AA287352 AA279767 AI479143 AA863044 AA134489 H49266 L32048 |
| 412668 | 33230_1 | AK057749 AI701055 AA115476 AI633570 AI435607 AW173392 AI092468 AI989318 AA833891 AW295964 AI804107 AI767415 AI473818 |
| | | AI076758 AA278949 AA428547 N31385 H82560 AA464804 AA809073 AA832476 AW295298 BF108690 AI492243 N22394 AA487175 |
| | | AA431891 AW070941 AA278823 AA707840 AI346067 AI184307 AA936342 AI288633 AA425697 AI435960 AI301433 AA993394 AI917652 |
| | | AA487234 AA490964 AA937925 AI301378 AI783840 AI093652 AA723864 BF001378 BI818971 BI762256 AW957064 BG720359 BI821176 |
| | | BI906440 AI472868 AL523506 BF962934 BF960650 BI837618 BF956256 AA487349 BF961475 AA354431 AA115452 AA491157 N75632 |
| | | BF962141 AL562216 BI754640 N24091 R00062 AI963686 T56529 AU185624 T56460 H97658 BI752843 BI819132 AA210796 |
| 443547 | 137089_2 | AV645808 AA701657 AW271273 AI796734 AI472316 AI017531 AI061178 BF109096 AA548964 N83805 AAI31648 AA156589 BE708349 |
| | | AW952494 D30877 AV684717 Z24837 F00167 BF576150 T63841 R78995 N87474 |
| 421341 | 1407_1 | NM_007329 AF159456 AJ243212 AJ297935 AA295769 NM_017579 AJ243224 AI492875 AI796676 AI749838 AA918144 AI814590 |
| | | AI923531 BF513992 AI720725 AI150879 AI279072 AW612904 AI492104 AI284510 AI141231 AA613554 AW662148 AW769047 AA565985 |
| | | AW612888 AU100513 BG955585 BG955588 AA295763 BE829414 BF760645 BG954398 AA295332 AA295795 BE932867 AW769569 |
| | | T89953 BE934311 |
| 448425 | 506563_1 | AW005628 AI500359 AW275593 |
| 451742 | 15505_1 | BC017986 AI129314 BF594240 N80108 AI273593 AI292203 AI341883 AI093196 AI090550 AI144158 AW613047 AL043865 N78951 D44679 |
| | | BE349232 AW511871 AL537825 AL134066 F13505 AW954583 AA351161 T77609 BG283250 C15501 D81753 R60790 F04232 AL537824 |
| | | AI096710 AI307186 AA458658 AW614225 AI804466 BF446326 AI799735 BF591932 AW129277 AI252220 AI217036 AW316545 AI287987 |
| | | AI880082 H15099 N89711 AA054086 F26355 AI362724 N80775 Z39382 AI910523 T33415 R42392 AA455356 AA975079 AA894809 |
| | | T33395 F03344 BF726212 N53354 AA019494 AA311664 F10695 R41849 AA019744 AW299413 AI372486 AI384058 N98737 N64675 |
| | | AI953492 AI372484 R39245 W31177 BF679932 BF377203 BF977609 BI768322 BI764551 |
| 453143 | 10116_6 | BG542081 BF793365 AI371013 AI147536 AW005418 AA416767 AI083516 AI698032 AA410929 AI936116 AI079893 AA747741 BF940413 |
| | | BM007681 F12285 N75819 AI971415 AA032249 AW867908 AW867914 AI520867 BG990651 AA570507 AA036654 AA063585 AA873147 |
| | | AI538117 AA382234 T66232 BE272411 AA834031 BG122734 BI769788 W55850 AW879266 |

Pkey: Unique Eos probeset identifier number
CAT number: Gene cluster number
Accession: Genbank accession numbers

TABLE 5C

| Pkey | Ref | Strand | Nt_position |
|---|---|---|---|
| 403593 | 6862650 | Minus | 62554-62712, 69449-69602 |
| 404246 | 7406725 | Plus | 82477-82628, 82721-82817, |
| | | | 82910-83071, 8314 |
| 401929 | 3810670 | Minus | 3167-3286, 4216-4310 |
| 405443 | 7408143 | Plus | 90716-90887, 101420-101577 |
| 402575 | 9884830 | Minus | 109742-109883 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 6A
About 1251 genes upregulated in lung metastases to the brain relative to normal lung

| Pkey | ExAccn | UniGeneID | UniGene Title | R1 | SEQ ID NO(s): |
|---|---|---|---|---|---|
| 406690 | M29540 | Hs.220529 | carcinoembryonic antigen-related cell ad | 55.34 | 4748 86 |
| 412719 | AW016610 | Hs.816 | ESTs | 31.39 | 663 |
| 422487 | AJ010901 | Hs.198267 | mucin 4, tracheobronchial | 29.00 | 1649 5152 |
| 409103 | AF251237 | Hs.112208 | XAGE-1 protein | 27.98 | 333 4812 |
| 444381 | BE387335 | Hs.283713 | hypothetical protein BC014245 | 27.21 | 3593 |
| 422963 | M79141 | Hs.13234 | sphingosine 1-phosphate phosphohydrolase | 26.63 | 1701 |
| 426682 | AV660038 | Hs.2056 | UDP glycosyltransferase 1 family, polype | 24.99 | 2086 |
| 414430 | AI346201 | Hs.76118 | ubiquitin carboxyl-terminal esterase L1 | 23.78 | 828 |
| 433447 | U29195 | Hs.3281 | neuronal pentraxin II | 23.05 | 2764 5536 |
| 429500 | X78565 | Hs.289114 | hexabrachion (tenascin C, cytotactin) | 21.72 | 2380 5410 |
| 411908 | L27943 | Hs.72924 | cytidine deaminase | 21.57 | 4869 585 |
| 409632 | W74001 | Hs.55279 | serine (or cysteine) proteinase inhibito | 20.87 | 390 |
| 431566 | AF176012 | Hs.260720 | J domain containing protein 1 | 19.92 | 2568 5479 |
| 425057 | AA826434 | Hs.1619 | achaete-scute complex (Drosophila) homol | 19.18 | 1915 |
| 456508 | AA502764 | Hs.123469 | ESTs, Weakly similar to AF208855 1 BM-01 | 19.04 | 4547 |
| 419078 | M93119 | Hs.89584 | insulinoma-associated 1 | 18.86 | 1272 5036 |
| 415906 | AI751357 | Hs.288741 | Homo sapiens cDNA: FLJ22256 fis, clone H | 18.81 | 959 |
| 419875 | AA853410 | Hs.93557 | proenkephalin | 18.39 | 1365 |
| 455601 | AI368680 | Hs.816 | SRY (sex determining region Y)-box 2 | 17.60 | 4515 |
| 428839 | AI767756 | Hs.82302 | Homo sapiens cDNA FLJ14814 fis, clone NT | 17.09 | 2310 |
| 439897 | NM_015310 | Hs.6763 | KIAA0942 protein | 17.06 | 3241 5600 |
| 417433 | BE270266 | Hs.82128 | 5T4 oncofetal trophoblast glycoprotein | 16.89 | 1094 |
| 449230 | BE613348 | | melanoma cell adhesion molecule | 16.73 | 4049 |
| 452838 | U65011 | Hs.30743 | preferentially expressed antigen in mela | 16.51 | 4353 5765 |
| 431958 | X63629 | Hs.2877 | cadherin 3, type 1, P-cadherin (placenta | 16.43 | 2621 5498 |
| 453857 | AL080235 | Hs.35861 | Ras-induced senescence 1 (RIS1) | 16.32 | 4437 5785 |
| 430130 | AL137311 | Hs.234074 | Homo sapiens mRNA; cDNA DKFZp761G02121 ( | 15.92 | 2445 5436 |
| 429228 | AI553633 | | hypothetical protein MGC33630 | 15.72 | 2340 |
| 424922 | BE386547 | Hs.217112 | hypothetical protein MGC10825 | 15.68 | 1900 |
| 447329 | BE090517 | | ESTs, Moderately similar to ALU8_HUMAN A | 15.67 | 3842 |
| 408393 | AW015318 | Hs.23165 | ESTs | 15.64 | 263 |
| 436217 | T53925 | Hs.107 | fibrinogen-like 1 | 15.60 | 2968 |
| 421948 | L42583 | Hs.334309 | keratin 6A | 15.17 | 1583 5130 |
| 452240 | AI591147 | Hs.61232 | ESTs | 15.02 | 4286 |
| 418738 | AW388633 | Hs.6682 | solute carrier family 7, (cationic amino | 14.89 | 1234 |
| 452487 | AW207659 | Hs.6630 | Homo sapiens cDNA FLJ13329 fis, clone OV | 14.82 | 4317 |
| 422440 | NM_004812 | Hs.116724 | aldo-keto reductase family 1, member B10 | 14.77 | 1647 5151 |
| 424001 | W67883 | Hs.137476 | paternally expressed 10 | 14.72 | 1788 |
| 408949 | AF189011 | Hs.49163 | putative ribonuclease III | 14.55 | 319 4809 |
| 431933 | AI187057 | Hs.132554 | ESTs | 14.54 | 2618 |
| 452747 | BE153855 | Hs.61460 | lg superfamily receptor LNIR | 14.48 | 4341 |
| 423634 | AW959908 | Hs.1690 | heparin-binding growth factor binding pr | 14.44 | 1762 |
| 432467 | T03667 | Hs.239388 | Human DNA sequence from clone RP1-304B14 | 14.36 | 2671 |
| 449722 | BE280074 | Hs.23960 | cyclin B1 | 14.29 | 4079 |
| 412490 | AW803564 | Hs.288850 | Homo sapiens cDNA: FLJ22528 fis, clone H | 14.27 | 637 |
| 406399 | | | NM_003122*:Homo sapiens serine protease | 14.16 | 4743 78 |
| 423073 | BE252922 | Hs.123119 | MAD (mothers against decapentaplegic, Dr | 13.94 | 1715 |
| 418329 | AW247430 | Hs.84152 | cystathionine-beta-synthase | 13.91 | 1186 |
| 428450 | NM_014791 | Hs.184339 | KIAA0175 gene product | 13.86 | 2259 5359 |
| 427871 | AW992405 | Hs.59622 | Homo sapiens, clone IMAGE:3507281, mRNA, | 13.56 | 2202 |
| 428342 | AI739168 | | Homo sapiens cDNA FLJ13458 fis, clone PL | 13.48 | 2244 |
| 426283 | NM_003937 | Hs.169139 | kynureninase (L-kynurenine hydrolase) | 13.45 | 2048 5297 |
| 411305 | BE241596 | Hs.69547 | myelin basic protein | 13.41 | 546 |
| 407137 | T97307 | | gb:ye53h05.s1 Soares fetal liver spleen | 13.41 | 128 |
| 410082 | AA081594 | Hs.158311 | Musashi (Drosophila) homolog 1 | 13.13 | 442 |
| 433485 | AI493076 | Hs.306098 | aldo-keto reductase family 1, member C2 | 13.09 | 2766 |
| 434826 | AF155661 | Hs.22265 | pyruvate dehydrogenase phosphatase | 13.05 | 2871 5556 |
| 425234 | AW152225 | Hs.165909 | ESTs, Weakly similar to I38022 hypotheti | 13.05 | 1940 |
| 437762 | T78028 | Hs.154679 | synaptotagmin I | 12.98 | 3088 |
| 427528 | AU077143 | Hs.179565 | minichromosome maintenance deficient (S. | 12.91 | 2161 |
| 426514 | BE616633 | Hs.170195 | bone morphogenetic protein 7 (osteogenic | 12.82 | 2073 |
| 424308 | AW975531 | Hs.154443 | minichromosome maintenance deficient (S. | 12.72 | 1827 |
| 416655 | AW968613 | Hs.79428 | BCL2/adenovirus E1B 19 kD-interacting pro | 12.61 | 1010 |
| 427344 | NM_000869 | Hs.2142 | 5-hydroxytryptamine (serotonin) receptor | 12.58 | 2145 5336 |
| 432441 | AW292425 | Hs.163484 | intron of hepatocyte nuclear factor-3 al | 12.57 | 2668 |
| 429170 | NM_001394 | Hs.2359 | dual specificity phosphatase 4 | 12.55 | 2336 5392 |
| 446080 | AI221741 | Hs.117777 | ESTs | 12.54 | 3723 |
| 414683 | S78296 | Hs.76888 | hypothetical protein MGC12702 | 12.54 | 4923 862 |
| 449845 | AW971183 | Hs.6019 | DnaJ (Hsp40) homolog, subfamily C, membe | 12.25 | 4088 |
| 410442 | X73424 | Hs.63788 | propionyl Coenzyme A carboxylase, beta p | 12.20 | 479 4848 |
| 422511 | AU076442 | Hs.117938 | collagen, type XVII, alpha 1 | 12.10 | 1650 |
| 442547 | AA306997 | Hs.217484 | ESTs, Weakly similar to ALU1_HUMAN ALU S | 11.92 | 3439 |
| 412446 | AI768015 | | ESTs | 11.86 | 633 |
| 414219 | W20010 | Hs.75823 | ALL1-fused gene from chromosome 1q | 11.78 | 804 |
| 414825 | X06370 | Hs.77432 | epidermal growth factor receptor (avian | 11.74 | 4930 882 |
| 422880 | AF228704 | Hs.193974 | glutathione reductase | 11.73 | 1689 5161 |
| 446469 | BE094848 | Hs.15113 | homogentisate 1,2-dioxygenase (homogenti | 11.65 | 3753 |
| 412636 | NM_004415 | | desmoplakin (DPI, DPII) | 11.63 | 4882 652 |
| 452099 | BE612992 | Hs.27931 | hypothetical protein FLJ10607 similar to | 11.54 | 4270 |
| 458692 | BE549905 | Hs.231754 | ESTs | 11.52 | 4633 |
| 431211 | M86849 | Hs.323733 | gap junction protein, beta 2, 26 kD (conn | 11.42 | 2540 5473 |
| 413554 | AA319146 | Hs.75426 | secretogranin II (chromogranin C) | 11.30 | 734 |
| 442432 | BE093589 | Hs.38178 | hypothetical protein FLJ23468 | 11.28 | 3429 |
| 429922 | Z97630 | Hs.226117 | H1 histone family, member 0 | 11.22 | 2427 5430 |
| 403149 | | | NM_001450:Homo sapiens four and a half L | 11.20 | 42 4711 |
| 424098 | AF077374 | Hs.139322 | small proline-rich protein 3 | 11.15 | 1804 5199 |
| 408298 | AI745325 | Hs.271923 | Homo sapiens cDNA: FLJ22785 fis, clone K | 11.13 | 253 |
| 426539 | AB011155 | Hs.170290 | discs, large (Drosophila) homolog 5 | 11.09 | 2076 5308 |
| 432460 | H12912 | Hs.274691 | adenylate kinase 3 | 11.06 | 2670 |
| 427398 | AW390020 | Hs.20415 | chromosome 21 open reading frame 11 | 11.02 | 2151 |
| 433339 | AF019226 | Hs.8036 | glioblastoma overexpressed | 10.98 | 2756 |
| 436420 | AA443966 | Hs.31595 | ESTs | 10.97 | 2984 |
| 412140 | AA219691 | Hs.73625 | RAB6 interacting, kinesin-like (rabkines | 10.97 | 613 |
| 434001 | AW950905 | Hs.3697 | angiotensinogen | 10.97 | 2804 |
| 409361 | NM_005982 | Hs.54416 | sine oculis homeobox (Drosophila) homolo | 10.94 | 364 4823 |
| 439963 | AW247529 | Hs.6793 | platelet-activating factor acetylhydrola | 10.87 | 3250 |
| 407833 | AW955632 | Hs.66666 | ESTs, Weakly similar to S 19560 proline-r | 10.84 | 204 |
| 433160 | AW207002 | Hs.134342 | TASP for testis-specific adriamycin sens | 10.78 | 2742 |
| 426827 | AW067805 | Hs.172665 | methylenetetrahydrofolate dehydrogenase | 10.64 | 2104 |
| 446342 | BE298665 | Hs.14846 | solute carrier family 7 (cationic amino | 10.58 | 3746 |
| 410337 | M83822 | Hs.62354 | cell division cycle 4-like | 10.58 | 465 4845 |
| 416854 | H40164 | Hs.80296 | Purkinje cell protein 4 | 10.58 | 1031 |
| 418327 | U70370 | Hs.84136 | paired-like homeodomain transcription fa | 10.55 | 1185 5009 |
| 425071 | NM_013989 | Hs.154424 | deiodinase, iodothyronine, type II | 10.53 | 1916 5244 |
| 409041 | AB033025 | Hs.50081 | Hypothetical protein, XP_051860 (KIAA119 | 10.52 | 327 4811 |
| 436291 | BE568452 | Hs.344037 | protein regulator of cytokinesis 1 | 10.50 | 2975 |
| 446639 | AI016826 | Hs.342148 | ESTs | 10.48 | 3774 |
| 421110 | AJ250717 | Hs.1355 | cathepsin E | 10.47 | 1481 5094 |
| 417308 | H60720 | Hs.81892 | KIAA0101 gene product | 10.47 | 1079 |
| 428931 | AA994979 | Hs.98967 | ATPase, H()-transporting, lysosomal, non | 10.46 | 2317 |
| 425465 | L18964 | Hs.1904 | protein kinase C, iota | 10.42 | 1969 5266 |
| 423472 | AF041260 | Hs.129057 | breast carcinoma amplified sequence 1 | 10.40 | 1749 5181 |
| 433001 | AF217513 | Hs.279905 | clone HQ0310 PRO0310p1 | 10.39 | 2719 5521 |
| 429663 | M68874 | Hs.211587 | phospholipase A2, group IVA (cytosolic, | 10.36 | 2404 5422 |
| 424378 | W28020 | Hs.167988 | neural cell adhesion molecule 1 | 10.33 | 1837 |
| 447033 | AI357412 | Hs.157601 | Predicted gene: Eos cloned; secreted w/V | 10.29 | 3814 |
| 413517 | N76712 | Hs.44829 | ESTs, Weakly similar to I38022 hypotheti | 10.27 | 730 |
| 422163 | AF027208 | Hs.112360 | prominin (mouse)-like 1 | 10.22 | 1611 5140 |
| 436469 | AK001455 | Hs.5198 | Down syndrome critical region gene 2 | 10.21 | 2986 |
| 409187 | AF154830 | Hs.50966 | carbamoyl-phosphate synthetase 1, mitoch | 10.21 | 347 4816 |
| 428966 | AF059214 | Hs.194687 | cholesterol 25-hydroxylase | 10.20 | 2320 5385 |
| 443683 | BE241717 | Hs.9676 | uncharacterized hypothalamus protein HT0 | 10.16 | 3539 |
| 426793 | X89887 | Hs.172350 | HIR (histone cell cycle regulation defec | 10.13 | 2100 5321 |
| 409269 | AA576953 | Hs.22972 | steroid 5 alpha-reductase 2-like; H5AR g | 10.09 | 358 |
| 428862 | NM_000346 | Hs.2316 | SRY (sex determining region Y)-box 9 (ca | 10.05 | 2313 5382 |
| 414869 | AA157291 | Hs.21479 | ubinuclein 1 | 9.98 | 885 |
| 407633 | NM_007069 | Hs.37189 | similar to rat HREV107 | 9.97 | 173 4776 |
| 410817 | AI262789 | Hs.93659 | protein disulfide isomerase related prot | 9.95 | 515 |
| 442355 | AA456539 | Hs.8262 | lysosomal-associated membrane protein 2 | 9.91 | 3424 |
| 448474 | AI792014 | Hs.13809 | hypothetical protein FLJ10648 | 9.87 | 3972 |
| 430589 | AJ002744 | Hs.246315 | UDP-N-acetyl-alpha-D-galactosamine:polyp | 9.84 | 2497 5457 |
| 456844 | AI264155 | Hs.152981 | CDP-diacylglycerol synthase (phosphatida | 9.83 | 4562 |
| 425322 | U63630 | Hs.155637 | protein kinase, DNA-activated, catalytic | 9.83 | 1950 5255 |
| 416294 | D86980 | Hs.79170 | KIAA0227 protein | 9.81 | 4958 984 |
| 446204 | AI279809 | Hs.150019 | ESTs | 9.77 | 3735 |
| 424954 | NM_000546 | Hs.1846 | tumor protein p53 (Li-Fraumeni syndrome) | 9.75 | 1901 5238 |
| 426559 | AB001914 | Hs.170414 | paired basic amino acid cleaving system | 9.73 | 2078 5309 |
| 441020 | W79283 | Hs.35962 | ESTs | 9.68 | 3325 |
| 431797 | BE169641 | Hs.270134 | hypothetical protein FLJ20280 | 9.66 | 2601 |
| 407355 | AA846203 | Hs.193974 | ESTs, Weakly similar to ALU1_HUMAN ALU S | 9.66 | 155 |
| 446839 | BE091926 | Hs.16244 | mitotic spindle coiled-coil related prof | 9.65 | 3794 |
| 434063 | AA018893 | Hs.3727 | unr-interacting protein | 9.63 | 2811 |
| 432886 | BE159028 | Hs.279704 | chromatin accessibility complex 1 | 9.57 | 2708 |
| 449349 | AI825386 | | hypothetical protein FLJ21939 similar to | 9.56 | 4057 |
| 453884 | AA355925 | Hs.36232 | KIAA0186 gene product | 9.54 | 4444 |
| 412519 | AA196241 | Hs.73980 | troponin T1, skeletal, slow | 9.53 | 641 |
| 428004 | AA449563 | Hs.151393 | glutamate-cysteine ligase, catalytic sub | 9.51 | 2213 |
| 440274 | R24595 | Hs.7122 | scrapie responsive protein 1 | 9.49 | 3275 |
| 423732 | AF058056 | Hs.132183 | solute carrier family 16 (monocarboxylic | 9.46 | 1770 5185 |
| 427674 | NM_003528 | Hs.2178 | H2B histone family, member Q | 9.43 | 2177 5342 |
| 441633 | AW958544 | Hs.112242 | normal mucosa of esophagus specific 1 | 9.42 | 3363 |
| 413753 | U17760 | Hs.75517 | laminin, beta 3 (nicein (125 kD), kalinin | 9.41 | 4906 750 |
| 452046 | AB018345 | Hs.27657 | KIAA0802 protein | 9.41 | 4266 5754 |
| 434061 | AW024973 | Hs.283675 | NPD009 protein | 9.40 | 2810 |
| 424572 | M19650 | Hs.179600 | 2′,3′-cyclic nucleotide 3′ phosphodieste | 9.40 | 1859 5218 |
| 435937 | AA830893 | Hs.119769 | ESTs | 9.35 | 2953 |
| 447818 | W79940 | Hs.21906 | Homo sapiens clone 24670 mRNA sequence | 9.34 | 3909 |
| 431374 | BE258532 | Hs.251871 | CTP synthase | 9.31 | 2551 |
| 431183 | NM_006855 | Hs.250696 | KDEL (Lys-Asp-Glu-Leu) endoplasmic retic | 9.30 | 2537 5472 |
| 421535 | AB002359 | Hs.105478 | phosphoribosylformylglycinamidine syntha | 9.28 | 1535 5115 |
| 407896 | D76435 | Hs.41154 | Zic family member 1 (odd-paired Drosophi | 9.28 | 213 4785 |
| 443715 | AI583187 | Hs.9700 | cyclin E1 | 9.26 | 3544 |
| 431726 | NM_015361 | Hs.268053 | KIAA0029 protein | 9.26 | 2592 5489 |
| 451807 | W52854 | | hypothetical protein FLJ23293 similar to | 9.23 | 4249 |
| 438461 | AW075485 | Hs.286049 | phosphoserine aminotransferase | 9.20 | 3141 |
| 435854 | AJ278120 | Hs.4996 | putative ankyrin-repeat containing prote | 9.19 | 2949 5571 |
| 408437 | AW957744 | Hs.278469 | lacrimal proline rich protein | 9.18 | 267 |
| 452576 | AB023177 | Hs.29900 | KIAA0960 protein | 9.11 | 4327 5760 |
| 450447 | AF212223 | Hs.25010 | hypothetical protein P15-2 | 9.08 | 4139 5730 |
| 427051 | BE178110 | Hs.173374 | Homo sapiens cDNA FLJ10500 fis, clone NT | 9.06 | 2123 |
| 414368 | W70171 | Hs.75939 | uridine monophosphate kinase | 9.06 | 818 |
| 446945 | AI193115 | Hs.16611 | tumor protein D52-like 1 | 9.05 | 3805 |
| 446873 | AI554439 | | ESTs | 9.01 | 3797 |
| 450377 | AB033091 | | KIAA1265 protein | 9.00 | 4134 5729 |
| 431707 | R21326 | Hs.267905 | hypothetical protein FLJ10422 | 9.00 | 2587 |
| 417733 | AL048678 | Hs.82503 | H. sapiens mRNA for 3'UTR of unknown prot | 8.94 | 1119 |
| 428987 | NM_004751 | Hs.194710 | glucosaminyl (N-acetyl) transferase 3, m | 8.93 | 2324 5386 |
| 433212 | BE218049 | Hs.121820 | ESTs | 8.93 | 2749 |
| 448520 | AB002367 | Hs.21355 | doublecortin and CaM kinase-like 1 | 8.91 | 3980 5710 |
| 424800 | AL035588 | Hs.153203 | MyoD family inhibitor | 8.90 | 1888 5232 |
| 451752 | AB032997 | | KIAA1171 protein | 8.87 | 4247 5750 |
| 401197 | | | ENSP00000229263*: HSPC213. | 8.79 | |
| 442772 | AW503680 | Hs.5957 | Homo sapiens clone 24416 mRNA sequence | 8.79 | 3468 |
| 411800 | N39342 | Hs.103042 | microtubule-associated protein 1B | 8.79 | 579 |
| 429973 | AI423317 | Hs.164680 | ESTs | 8.75 | 2432 |
| 450937 | R49131 | Hs.26267 | ATP-dependant interferon response protei | 8.72 | 4177 |
| 418721 | NM_002731 | Hs.87773 | protein kinase, cAMP-dependent, catalyti | 8.72 | 1231 5027 |
| 400247 | | | Eos Control | 8.71 | |
| 420153 | N22120 | Hs.75277 | hypothetical protein FLJ13910 | 8.68 | 1389 |
| 436895 | AF037335 | Hs.5338 | carbonic anhydrase XII | 8.68 | 3019 5582 |
| 457465 | AW301344 | Hs.122908 | DNA replication factor | 8.67 | 4592 |
| 441031 | AI110684 | Hs.7645 | fibrinogen, B beta polypeptide | 8.61 | 3329 |
| 419239 | AA468183 | Hs.184598 | Homo sapiens cDNA: FLJ23241 fis, clone C | 8.60 | 1292 |
| 427982 | NM_016156 | Hs.181326 | KIAA1073 protein | 8.60 | 2210 5349 |
| 415914 | AA306033 | Hs.78915 | GA-binding protein transcription factor, | 8.60 | 960 |
| 422656 | AI870435 | Hs.1569 | LIM homeobox protein 2 | 8.53 | 1668 |
| 443247 | BE614387 | Hs.333893 | c-Myc target JPO1 | 8.51 | 3513 |
| 410102 | AW248508 | Hs.279727 | ESTs; homologue of PEM-3 [Ciona savignyi | 8.50 | 446 |
| 401451 | | | NM_004496*:Homo sapiens hepatocyte nucle | 8.50 | 27 4697 |
| 441680 | AW444598 | Hs.7940 | RAP1, GTP-GDP dissociation stimulator 1 | 8.48 | 3368 |
| 452367 | U71207 | Hs.29279 | eyes absent (Drosophila) homolog 2 | 8.48 | 4300 5757 |
| 444172 | BE147740 | | ESTs, Moderately similar I38022 hypot | 8.48 | 3580 |
| 414883 | AA926960 | | CDC28 protein kinase 1 | 8.47 | 887 |
| 412652 | AI801777 | | ESTs | 8.46 | 655 |
| 426788 | U66615 | Hs.172280 | SWI/SNF related, matrix associated, acti | 8.45 | 2099 5320 |
| 418827 | BE327311 | Hs.47166 | HT021 | 8.40 | 1245 |
| 415138 | C18356 | Hs.295944 | tissue factor pathway inhibitor 2 | 8.39 | 910 |
| 453507 | AF083217 | Hs.33085 | WD repeat domain 3 | 8.36 | 4414 5778 |
| 407944 | R34008 | Hs.239727 | desmocollin 2 | 8.34 | 218 |
| 407168 | R45175 | Hs.117183 | ESTs | 8.33 | 131 |
| 419631 | AW188117 | | popeye protein 3 | 8.31 | 1340 |
| 416000 | R82342 | Hs.79856 | ESTs, Weakly similar to S65657 alpha-1C- | 8.29 | 965 |
| 442426 | AI373062 | Hs.332938 | hypothetical protein MGC5370 | 8.29 | 3427 |
| 409977 | AW805510 | Hs.97056 | hypothetical protein FLJ21634 | 8.27 | 424 |
| 412482 | AI499930 | Hs.334885 | mitochondrial GTP binding protein | 8.24 | 636 |
| 429597 | NM_003816 | Hs.2442 | a disintegrin and metalloproteinase doma | 8.23 | 2392 5412 |
| 428340 | AF261088 | Hs.154721 | aconitase 1, soluble | 8.22 | 2243 5355 |
| 436557 | W15573 | Hs.5027 | ESTs, Weakly similar to A47582 B-cell gr | 8.20 | 2993 |
| 416602 | NM_006159 | Hs.79389 | Protein kinase C-binding protein NELL2 | 8.14 | 1006 4965 |
| 421305 | BE397354 | Hs.324830 | diptheria toxin resistance protein requi | 8.14 | 1505 |
| 453439 | AI572438 | Hs.32976 | guanine nucleotide binding protein 4 | 8.14 | 4406 |
| 414761 | AU077228 | Hs.77256 | enhancer of zeste (Drosophila) homolog 2 | 8.12 | 875 |
| 441362 | BE614410 | Hs.23044 | RAD51 (S. cerevisiae) homolog (E coil Re | 8.12 | 3347 |
| 439924 | AI985897 | Hs.125293 | ESTs | 8.11 | 3242 |
| 408576 | NM_003542 | Hs.46423 | H4 histone family, member G | 8.10 | 280 4800 |
| 429343 | AK000785 | Hs.199480 | Homo sapiens, Similar to epsin 3, clone | 8.10 | 2356 5402 |
| 417720 | AA205625 | Hs.208067 | ESTs | 8.08 | 1116 |
| 426167 | AF039023 | Hs.167496 | RAN binding protein 6 | 8.06 | 2034 5295 |
| 432426 | AW973152 | Hs.31050 | ESTs | 8.06 | 2666 |
| 422956 | BE545072 | Hs.122579 | ECT2 protein (Epithelial cell transformi | 8.05 | 1698 |
| 415927 | AL120168 | Hs.78919 | Kell blood group precursor (McLeod pheno | 8.03 | 961 |
| 400409 | AF153341 | | Homo sapiens winged helix/forkhead trans | 8.02 | 15 4687 |
| 419743 | AW408762 | Hs.5957 | Homo sapiens clone 24416 mRNA sequence | 8.02 | 1356 |
| 430937 | X53463 | Hs.2704 | glutathione peroxidase 2 (gastrointestin | 8.01 | 2522 5466 |
| 417576 | AA339449 | Hs.82285 | phosphoribosylglycinamide formyltransfer | 8.01 | 1102 |
| 433980 | AA137152 | Hs.286049 | phosphoserine aminotransferase | 8.00 | 2803 |
| 405770 | | | NM_002362:Homo sapiens melanoma antigen, | 8.00 | 4740 74 |
| 434423 | NM_006769 | Hs.3844 | LIM domain only 4 | 8.00 | 2839 5548 |
| 447414 | D82343 | Hs.74376 | neuroblastoma (nerve tissue) protein | 7.99 | 3859 5687 |
| 407748 | AL079409 | Hs.38176 | KIAA0606 protein; SCN Circadian Oscillat | 7.99 | 188 |
| 422150 | AI867118 | | calpastatin | 7.98 | 1609 |
| 417333 | AL157545 | Hs.173179 | bromodomain and PHD finger containing, 3 | 7.97 | 1081 |
| 416391 | AI878927 | Hs.79284 | mesoderm specific transcript (mouse) hom | 7.94 | 992 |
| 418304 | AA215702 | | gb:zr97g10.rl NCl_CGAP_GCB1 Homo sapiens | 7.91 | 1178 |
| 430066 | AI929659 | Hs.237825 | signal recognition particle 72 kD | 7.90 | 2442 |
| 433862 | D86960 | Hs.3610 | KIAA0205 gene product | 7.84 | 2793 5542 |
| 430304 | AL122071 | Hs.238927 | Homo sapiens mRNA; cDNA DKFZp434H1235 (f | 7.81 | 2467 5442 |
| 446525 | AW967069 | Hs.211556 | hypothetical protein MGC5487 | 7.80 | 3758 |
| 414217 | AI309298 | Hs.279898 | Homo sapiens cDNA: FLJ23165 fis, clone L | 7.78 | 803 |
| 438549 | BE386801 | Hs.21858 | trinucleotide repeat containing 3 | 7.78 | 3147 |
| 457211 | AW972565 | Hs.32399 | ESTs, Weakly similar to S51797 vasodilat | 7.77 | 4583 |
| 413437 | BE313164 | Hs.75361 | gene from NF2/meningioma region of 22q12 | 7.77 | 722 |
| 418164 | AI761820 | Hs.41074 | ESTs, Weakly similar to I39294 McLeod sy | 7.76 | 1165 |
| 448826 | AI580252 | Hs.293246 | ESTs, Weakly similar to putative p150 [H | 7.74 | 4012 |
| 437271 | AL137445 | Hs.28846 | Homo sapiens mRNA; cDNA DKFZp566O134 (fr | 7.71 | 3052 |
| 450325 | AI935962 | Hs.91973 | ESTs | 7.69 | 4129 |
| 429276 | AF056085 | Hs.198612 | G protein-coupled receptor 51 | 7.67 | 2346 5397 |
| 433201 | AB040896 | Hs.21104 | KIAA1463 protein | 7.66 | 2747 5532 |
| 420309 | AW043637 | Hs.21766 | ESTs, Weakly similar to ALU5_HUMAN ALU S | 7.66 | 1408 |
| 427581 | NM_014788 | Hs.179703 | KIAA0129 gene product | 7.65 | 2167 5339 |
| 424834 | AK001432 | Hs.153408 | Homo sapiens cDNA FLJ10570 fis, clone NT | 7.65 | 1889 |
| 447342 | AI199268 | Hs.19322 | Homo sapiens, Similar to RIKEN cDNA 2010 | 7.65 | 3845 |
| 416783 | AA206186 | Hs.79889 | monocyte to macrophage differentiation-a | 7.64 | 1022 |
| 420039 | NM_004605 | Hs.94581 | sulfotransferase family, cytosolic, 2B, | 7.63 | 1377 5061 |
| 442332 | AI693251 | Hs.8248 | Target CAT | 7.63 | 3421 |
| 439941 | AI392640 | Hs.18272 | amino acid transporter system A1 | 7.63 | 3246 |
| 451061 | AW291487 | Hs.213659 | ESTs, Weakly similar to KIAA1357 protein | 7.62 | 4186 |
| 409757 | NM_001898 | Hs.123114 | cystatin SN | 7.62 | 403 4832 |
| 438523 | H66220 | Hs.278177 | ESTs | 7.61 | 3144 |
| 444670 | H58373 | Hs.332938 | hypothetical protein MGC5370 | 7.61 | 3618 |
| 446771 | AA128965 | Hs.60679 | TATA box binding protein (TBP)-associate | 7.61 | 3783 |
| 427944 | AA417878 | Hs.48401 | ESTs, Moderately similar to ALU8_HUMAN A | 7.60 | 2205 |
| 447343 | AA256641 | Hs.236894 | ESTs, Highly similar to S02392 alpha-2-m | 7.59 | 3846 |
| 418113 | AI272141 | Hs.83484 | SRY (sex determining region Y)-box 4 | 7.58 | 1161 |
| 407788 | BE514982 | Hs.38991 | S100 calcium-binding protein A2 | 7.57 | 195 |
| 408452 | AA054683 | Hs.192455 | ESTs, Weakly similar to ALU7_HUMAN ALU S | 7.54 | 268 |
| 428648 | AF052728 | Hs.188021 | potassium voltage-gated channel, subfami | 7.54 | 2279 5369 |
| 448209 | AW160489 | Hs.20709 | tetraspan 5 | 7.53 | 3951 |
| 432415 | T16971 | Hs.289014 | ESTs, Weakly similar to A43932 mucin 2 p | 7.53 | 2665 |
| 430294 | AI538226 | Hs.32976 | guanine nucleotide binding protein 4 | 7.50 | 2463 |
| 440700 | AW952281 | Hs.296184 | guanine nucleotide binding protein (G pr | 7.49 | 3313 |
| 425842 | AI587490 | Hs.159623 | NK-2 (Drosophila) homolog B | 7.47 | 2002 |
| 409799 | D11928 | Hs.76845 | phosphoserine phosphatase-like | 7.46 | 407 |
| 408524 | D87942 | Hs.46328 | fucosyltransferase 2 (secretor status in | 7.42 | 275 4799 |
| 433730 | AK002135 | Hs.3542 | hypothetical protein FLJ11273 | 7.41 | 2783 5540 |
| 444783 | AK001468 | Hs.62180 | anillin (Drosophila Scraps homolog), act | 7.39 | 3628 5645 |
| 450378 | AW249181 | Hs.198899 | ESTs, Weakly similar to T19873 hypotheti | 7.39 | 4135 |
| 452721 | AJ269529 | Hs.301871 | solute carrier family 37 (glycerol-3-pho | 7.39 | 4337 5761 |
| 422283 | AW411307 | Hs.114311 | CDC45 (cell division cycle 45, S.cerevis | 7.38 | 1626 |
| 409974 | BE174106 | Hs.225641 | hypothetical protein FLJ13171 | 7.38 | 423 |
| 426779 | AA384577 | Hs.93714 | ESTs, Weakly similar to T00365 hypotheti | 7.38 | 2097 |
| 424602 | AK002055 | Hs.151046 | hypothetical protein FLJ11193 | 7.36 | 1863 5219 |
| 431789 | H19500 | Hs.269222 | mitogen-activated protein kinase 4 | 7.36 | 2599 |
| 435538 | AB011540 | Hs.4930 | low density lipoprotein receptor-related | 7.35 | 2924 5565 |
| 437810 | BE246399 | | hypothetical protein | 7.35 | 3093 |
| 425843 | BE313280 | Hs.159627 | death associated protein 3 | 7.34 | 2003 |
| 424544 | M88700 | Hs.150403 | dopa decarboxylase (aromatic L-amino aci | 7.33 | 1858 5217 |
| 416426 | AA180256 | Hs.210473 | Homo sapiens cDNA FLJ14872 fis, clone PL | 7.32 | 996 |
| 412715 | NM_000947 | Hs.74519 | primase, polypeptide 2A (58 kD) | 7.32 | 4885 662 |
| 420552 | AK000492 | Hs.98806 | hypothetical protein | 7.29 | 1430 5081 |
| 409509 | AL036923 | Hs.322710 | ESTs | 7.29 | 379 |
| 439708 | AI761369 | Hs.59584 | hypothetical protein FLJ21144 | 7.29 | 3231 |
| 420281 | AI623693 | Hs.323494 | Predicted cation efflux pump | 7.29 | 1405 |
| 406972 | M32053 | | gb:Human H19 RNA gene, complete cds. | 7.28 | 115 |
| 446595 | T57448 | Hs.15467 | hypothetical protein FLJ20725 | 7.27 | 3768 |
| 412942 | AL120344 | Hs.75074 | mitogen-activated protein kinase-activat | 7.27 | 685 |
| 445098 | AL050272 | Hs.12305 | DKFZP566B183 protein | 7.26 | 3651 5651 |
| 446006 | NM_004403 | Hs.13530 | deafness, autosomal dominant 5 | 7.24 | 3717 5662 |
| 412935 | BE267045 | Hs.75064 | tubulin-specific chaperone c | 7.23 | 683 |
| 432103 | T15803 | Hs.272458 | protein phosphatase 3 (formerly 2B), cat | 7.22 | 2631 |
| 446619 | AU076643 | Hs.313 | secreted phosphoprotein 1 (osteopontin, | 7.20 | 3770 |
| 436009 | H57130 | Hs.120925 | ESTs | 7.20 | 2955 |
| 445413 | AA151342 | Hs.12677 | CGI-147 protein | 7.20 | 3675 |
| 417454 | NM_000202 | Hs.172458 | iduronate 2-sulfatase (Hunter syndrome) | 7.19 | 1097 4986 |
| 442445 | AA082665 | Hs.209561 | KIAA1715 protein | 7.18 | 3431 |
| 411975 | AI916058 | Hs.144583 | 3′UTR of: dead ringer (Drosophila)-like | 7.18 | 596 |
| 426501 | AW043782 | Hs.293616 | ESTs | 7.18 | 2072 |
| 426759 | AI590401 | Hs.21213 | ESTs | 7.17 | 2094 |
| 447644 | AW861622 | Hs.108646 | Homo sapiens cDNA FLJ14934 fis, clone PL | 7.16 | 3886 |
| 413654 | AA331881 | Hs.75454 | peroxiredoxin 3 | 7.16 | 745 |
| 445893 | AI610702 | Hs.202613 | ESTs, Weakly similar to TRHY_HUMAN TRICH | 7.12 | 3710 |
| 407252 | AA659037 | Hs.163780 | ESTs | 7.12 | 146 |
| 423242 | AL039402 | Hs.125783 | DEME-6 protein | 7.08 | 1730 |
| 426471 | M22440 | Hs.170009 | transforming growth factor, alpha | 7.08 | 2068 5305 |
| 418027 | AB037807 | Hs.83293 | hypothetical protein | 7.05 | 1149 5000 |
| 448497 | BE613269 | Hs.21893 | hypothetical protein DKFZp761N0624 | 7.05 | 3977 |
| 446636 | AC002563 | Hs.15767 | citron (rho-interacting, serine/threonin | 7.03 | 3773 5674 |
| 416498 | U33632 | Hs.79351 | potassium channel, subfamily K, member 1 | 7.02 | 4962 999 |
| 412452 | AA215731 | | suppression of tumorigenicity 5 | 7.02 | 634 |
| 413281 | AA861271 | Hs.222024 | transcription factor BMAL2 | 6.99 | 706 |
| 434540 | NM_016045 | Hs.3945 | CGI-107 protein | 6.98 | 2847 5549 |
| 445994 | NM_004724 | Hs.13512 | ZW10 (Drosophila) homolog, centromere/ki | 6.98 | 3716 5661 |
| 416815 | U41514 | Hs.80120 | UDP-N-acetyl-alpha-D-galactosamine:polyp | 6.97 | 1027 4968 |
| 418338 | NM_002522 | Hs.84154 | neuronal pentraxin I | 6.97 | 1189 5010 |
| 436389 | AI811706 | Hs.42733 | CHMP1.5 protein | 6.94 | 2981 |
| 418203 | X54942 | Hs.83758 | CDC28 protein kinase 2 | 6.93 | 1169 5003 |
| 418863 | AL135743 | Hs.25566 | ESTs, Weakly similar to 2004399A chromos | 6.93 | 1251 |
| 413551 | BE242639 | Hs.75425 | ubiquitin associated protein | 6.93 | 733 |
| 424505 | AA446131 | Hs.124918 | KIAA1795 protein | 6.92 | 1853 |
| 426359 | AA376409 | Hs.10862 | Homo sapiens cDNA: FLJ23313 fis, clone H | 6.91 | 2059 |
| 423453 | AW450737 | Hs.128791 | CGI-09 protein | 6.91 | 1748 |
| 450256 | AA286887 | Hs.24724 | MFH-amplified sequences with leucine-ric | 6.91 | 4120 |
| 443119 | AA312264 | Hs.7980 | hypothetical protein MGC12966 | 6.90 | 3498 |
| 410268 | AA316181 | Hs.61635 | six transmembrane epithelial antigen of | 6.90 | 461 |
| 406850 | AI624300 | Hs.172928 | collagen, type I, alpha 1 | 6.90 | 104 |
| 409619 | AK001015 | Hs.55220 | BCL2-associated athanogene 2 | 6.88 | 388 |
| 424381 | AA285249 | Hs.146329 | protein kinase Chk2 (CHEK2) | 6.86 | 1838 |
| 431548 | AI834273 | Hs.9711 | novel protein | 6.84 | 2564 |
| 409420 | Z15008 | Hs.54451 | laminin, gamma 2 (nicein (100 kD), kalini | 6.84 | 368 4824 |
| 453454 | AW052006 | | PRP4/STK/WD splicing factor | 6.83 | 4407 |
| 407807 | AL031427 | Hs.40094 | Human DNA sequence from clone 167A19 on | 6.82 | 198 |
| 445863 | R12234 | Hs.13396 | Homo sapiens clone 25028 mRNA sequence | 6.80 | 3703 |
| 442069 | AW664144 | Hs.297007 | membrane-bound transcription factor prot | 6.78 | 3393 |
| 428771 | AB028992 | Hs.193143 | KIAA1069 protein | 6.78 | 2295 5375 |
| 426141 | C05886 | Hs.293972 | ESTs | 6.77 | 2031 |
| 445817 | NM_003642 | Hs.13340 | histone acetyltransferase 1 | 6.77 | 3700 5659 |
| 408249 | AW271838 | Hs.44038 | pellino (Drosophila) homolog 2 | 6.76 | 249 |
| 438209 | AL120659 | Hs.6111 | aryl-hydrocarbon receptor nuclear transl | 6.75 | 3126 |
| 420164 | AW339037 | Hs.24908 | ESTs | 6.75 | 1390 |
| 417821 | BE245149 | Hs.82643 | protein tyrosine kinase 9 | 6.74 | 1126 |
| 432978 | AF126743 | Hs.279884 | DNAJ domain-containing | 6.73 | 2717 5520 |
| 428484 | AF104032 | Hs.184601 | solute carrier family 7 (cationic amino | 6.73 | 2265 5364 |
| 433409 | AI278802 | Hs.25661 | ESTs | 6.73 | 2761 |
| 427286 | AW732802 | Hs.2132 | epidermal growth factor receptor pathway | 6.72 | 2138 |
| 431770 | BE221880 | Hs.268555 | 5′-3′ exoribonuclease 2 | 6.72 | 2596 |
| 407635 | AW370213 | Hs.295232 | ESTs, Moderately similar to A46010 X-lin | 6.72 | 175 |
| 410240 | AL157424 | Hs.61289 | synaptojanin 2 | 6.72 | 459 |
| 401519 | | | C15000476*:gi\|12737279\|ref\|XP_012163.1\| | 6.71 | |
| 409557 | BE182896 | Hs.3686 | ESTs | 6.71 | 384 |
| 445800 | AA126419 | Hs.32944 | inositol polyphosphate-4-phosphatase, ty | 6.70 | 3699 |
| 415947 | U04045 | Hs.78934 | mutS (E. coli) homolog 2 (colon cancer, | 6.70 | 4955 962 |
| 426125 | X87241 | Hs.166994 | FAT tumor suppressor (Drosophila) homolo | 6.70 | 2029 5292 |
| 451418 | BE387790 | Hs.26369 | hypothetical protein FLJ20287 | 6.69 | 4221 |
| 428698 | AA852773 | Hs.334838 | KIAA1866 protein | 6.68 | 2283 |
| 429903 | AL134197 | Hs.93597 | cyclin-dependent kinase 5, regulatory su | 6.68 | 2425 |
| 433437 | U20536 | Hs.3280 | caspase 6, apoptosis-related cysteine pr | 6.66 | 2762 5535 |
| 411945 | AL033527 | Hs.92137 | L-myc-2 protein (MYCL2) | 6.65 | 4870 590 |
| 424140 | Z48051 | Hs.141308 | myelin oligodendrocyte glycoprotein | 6.65 | 1809 5200 |
| 413010 | AA393273 | Hs.75133 | transcription factor 6-like 1 (mitochond | 6.63 | 689 |
| 438915 | AA280174 | Hs.285681 | Williams-Beuren syndrome chromosome regi | 6.63 | 3178 |
| 410619 | BE512730 | Hs.65114 | keratin 18 | 6.62 | 498 |
| 410174 | AA306007 | Hs.59461 | DKFZP434C245 protein | 6.62 | 453 |
| 423250 | BE061916 | Hs.125849 | chromosome 8 open reading frame 2 | 6.62 | 1732 |
| 409542 | AA503020 | Hs.36563 | hypothetical protein FLJ22418 | 6.61 | 382 |
| 448664 | AI879317 | Hs.334691 | splicing factor 3a, subunit 1, 120 kD | 6.61 | 3994 |
| 429332 | AF030403 | Hs.199263 | Ste-20 related kinase | 6.61 | 2355 5401 |
| 421778 | AA428000 | Hs.283072 | actin related protein 2/3 complex, subun | 6.60 | 1563 |
| 450800 | BE395161 | Hs.1390 | proteasome (prosome, macropain) subunit, | 6.57 | 4167 |
| 433556 | W56321 | Hs.111460 | calcium/calmodulin-dependent protein kin | 6.55 | 2768 |
| 434263 | N34895 | Hs.79187 | ESTs | 6.54 | 2825 |
| 422967 | AL117526 | Hs.118164 | hypothetical protein FLJ12383 | 6.54 | 1703 5164 |
| 429183 | AB014604 | Hs.197955 | KIAA0704 protein | 6.53 | 2337 5393 |
| 428753 | AW939252 | Hs.192927 | hypothetical protein FLJ20251 | 6.53 | 2291 |
| 443991 | NM_002250 | Hs.10082 | potassium intermediate/small conductance | 6.53 | 3566 5632 |
| 442092 | AW578669 | | hypothetical protein FLJ12439 | 6.52 | 3397 |
| 442013 | AA506476 | Hs.82689 | Human DNA sequence from clone RP11-353C1 | 6.51 | 3388 |
| 414706 | AW340125 | Hs.76989 | KIAA0097 gene product | 6.51 | 865 |
| 434629 | AA789081 | Hs.4029 | glioma-amplified sequence-41 | 6.50 | 2854 |
| 452259 | AA317439 | Hs.28707 | signal sequence receptor, gamma (translo | 6.50 | 4290 |
| 428471 | X57348 | Hs.184510 | stratifin | 6.49 | 2262 5361 |
| 407378 | AA299264 | Hs.57776 | ESTs, Moderately similar to I38022 hypot | 6.49 | 160 |
| 416221 | BE513171 | Hs.79086 | mitochondrial ribosomal protein L3 | 6.49 | 982 |
| 426716 | NM_006379 | Hs.171921 | sema domain, immunoglobulin domain (Ig), | 6.47 | 2090 5315 |
| 442315 | AA173992 | Hs.7956 | ESTs, Moderately similar to ZN91_HUMAN Z | 6.47 | 3414 |
| 442571 | C06338 | Hs.165464 | ESTs | 6.46 | 3444 |
| 415156 | X84908 | Hs.78060 | phosphorylase kinase, beta | 6.45 | 4940 911 |
| 443180 | R15875 | Hs.258576 | claudin 12 | 6.45 | 3504 |
| 429413 | NM_014058 | Hs.201877 | DESC1 protein | 6.43 | 2366 5405 |
| 417348 | AI940507 | Hs.318526 | hypothetical protein FLJ12661 | 6.43 | 1084 |
| 407235 | D20569 | Hs.169407 | SAC2 (suppressor of actin mutations 2, y | 6.43 | 140 |
| 419175 | AW270037 | | KIAA0779 protein | 6.41 | 1286 |
| 438321 | AA576635 | Hs.6153 | CGI-48 protein | 6.41 | 3133 |
| 413582 | AW295647 | Hs.71331 | hypothetical protein MGC5350 | 6.41 | 736 |
| 429788 | U87791 | Hs.221040 | HBS1 (S. cerevisiae)-like | 6.40 | 2417 5426 |
| 410418 | D31382 | Hs.63325 | transmembrane protease, serine 4 | 6.40 | 476 |
| 410762 | AF226053 | Hs.66170 | HSKM-B protein | 6.39 | 4857 514 |
| 434614 | AI249502 | Hs.29669 | ESTs | 6.39 | 2852 |
| 411865 | AA248750 | Hs.12332 | ESTs | 6.39 | 582 |
| 416072 | AL110370 | Hs.79000 | growth associated protein 43 | 6.39 | 970 |
| 433345 | AI681545 | Hs.152982 | hypothetical protein FLJ13117 | 6.38 | 2757 |
| 412507 | L36645 | Hs.73964 | EphA4 | 6.38 | 4880 639 |
| 418004 | U37519 | Hs.87539 | aldehyde dehydrogenase 3 family, member | 6.37 | 1145 4997 |
| 422309 | U79745 | Hs.114924 | solute carrier family 16 (monocarboxylic | 6.36 | 1630 5146 |
| 415339 | NM_015156 | Hs.78398 | KIAA0071 protein | 6.36 | 4946 927 |
| 410340 | AW182833 | Hs.112188 | hypothetical protein FLJ13149 | 6.36 | 466 |
| 411352 | NM_002890 | Hs.758 | RAS p21 protein activator (GTPase activa | 6.35 | 4863 547 |
| 423739 | AA398155 | Hs.97600 | ESTs | 6.35 | 1771 |
| 419135 | R61448 | Hs.106728 | ESTs, Weakly similar to KIAA1353 protein | 6.33 | 1280 |
| 413450 | Z99716 | Hs.75372 | N-acetylgalactosaminidase, alpha- | 6.33 | 4901 723 |
| 447484 | AA464839 | Hs.292566 | hypothetical protein FLJ14697 | 6.33 | 3866 |
| 413063 | AL035737 | Hs.75184 | chitinase 3-like 1 (cartilage glycoprote | 6.33 | 692 |
| 419641 | BE170548 | Hs.118190 | Homo sapiens cDNA:FLJ21081 fis, clone C | 6.32 | 1342 |
| 400297 | AI127076 | Hs.306201 | hypothetical protein DKFZp564O1278 | 6.32 | 9 |
| 410963 | R78824 | Hs.44175 | KIAA0919 protein | 6.32 | 524 |
| 420234 | AW404908 | Hs.96038 | Ric (Drosophila)-like, expressed in many | 6.31 | 1399 |
| 408405 | AK001332 | Hs.44672 | hypothetical protein FLJ10470 | 6.30 | 264 4796 |
| 408331 | NM_007240 | Hs.44229 | dual specificity phosphatase 12 | 6.30 | 255 4793 |
| 451336 | AI264643 | Hs.3610 | ESTs | 6.29 | 4212 |
| 409327 | L41162 | Hs.53563 | collagen, type IX, alpha 3 | 6.29 | 361 4822 |
| 439211 | AI890347 | Hs.271923 | Homo sapiens cDNA:FLJ22785 fis, clone K | 6.27 | 3193 |
| 452223 | AA425467 | Hs.8035 | hypothetical protein MGC2827 | 6.25 | 4283 |
| 429925 | NM_000786 | Hs.226213 | cytochrome P450, 51 (lanosterol 14-alpha | 6.24 | 2428 |
| 420942 | H03514 | Hs.15589 | ESTs | 6.24 | 1467 |
| 411605 | AW006831 | | ESTs | 6.23 | 563 |
| 436299 | AK000767 | Hs.5111 | hypothetical protein FLJ20729 | 6.22 | 2977 5578 |
| 445903 | AI347487 | Hs.132781 | class I cytokine receptor | 6.22 | 3711 |
| 425836 | AW955696 | Hs.90960 | ESTs | 6.22 | 2000 |
| 431387 | AI878854 | Hs.252229 | v-maf musculoaponeurotic fibrosarcoma (a | 6.21 | 2552 |
| 437296 | AA350994 | Hs.20281 | KIAA1700 | 6.20 | 3057 |
| 414715 | AA587891 | Hs.904 | amylo-1,6-glucosidase, 4-alpha-glucanotr | 6.20 | 867 |
| 453204 | R10799 | Hs.191990 | ESTs | 6.18 | 4385 |
| 438452 | AI220911 | Hs.288959 | hypothetical protein FLJ20920 | 6.18 | 3139 |
| 434474 | AL042936 | Hs.211571 | holocytochrome c synthase (cytochrome c | 6.17 | 2842 |
| 404440 | | | NM_021048:Homo sapiens melanoma antigen, | 6.17 | 4721 54 |
| 430289 | AK001952 | Hs.238039 | hypothetical protein FLJ11090 | 6.16 | 2461 5440 |
| 426990 | AL044315 | Hs.173094 | Homo sapiens mRNA for KIAA1750 protein, | 6.15 | 2116 |
| 446494 | AA463276 | Hs.288906 | WW Domain-Containing Gene | 6.15 | 3755 |
| 453830 | AA534296 | Hs.20953 | ESTs | 6.15 | 4434 |
| 408713 | NM_001248 | Hs.47042 | ectonucleoside triphosphate diphosphohyd | 6.15 | 296 4804 |
| 423979 | AF229181 | Hs.136644 | CS box-containing WD protein | 6.15 | 1786 5191 |
| 412659 | AW753865 | Hs.74376 | olfactomedin related ER localized protei | 6.14 | 656 |
| 417601 | NM_014735 | Hs.82292 | KIAA0215 gene product | 6.14 | 1105 4991 |
| 422085 | AB018257 | Hs.288773 | zinc finger protein 294 | 6.14 | 1599 5135 |
| 417381 | AF164142 | Hs.82042 | solute carrier family 23 (nucleobase tra | 6.14 | 1089 4983 |
| 446390 | AA233393 | Hs.14992 | hypothetical protein FLJ11151 | 6.13 | 3749 |
| 441285 | NM_002374 | Hs.167 | microtubule-associated protein 2 | 6.12 | 3343 5616 |
| 409062 | AL157488 | Hs.50150 | Homo sapiens mRNA; cDNA DKFZp564B182 (fr | 6.12 | 329 |
| 440675 | AW005054 | Hs.279788 | ESTs, Weakly similar to KCC1_HUMAN CALCI | 6.11 | 3312 |
| 448072 | AI459306 | Hs.24908 | ESTs | 6.10 | 3940 |
| 430387 | AW372884 | Hs.240770 | nuclear cap binding protein subunit 2, 2 | 6.10 | 2479 |
| 424036 | AA770688 | | H2A histone family, member L | 6.10 | 1793 |
| 432908 | AI861896 | | ESTs | 6.10 | 2711 |
| 431512 | BE270734 | Hs.2795 | lactate dehydrogenase A | 6.09 | 2561 |
| 454293 | H49739 | Hs.134013 | ESTs, Moderately similar to HK61_HUMAN H | 6.09 | 4475 |
| 431452 | AI073641 | Hs.152372 | ESTs, Weakly similar to 2109260A B cell | 6.09 | 2556 |
| 447197 | R36075 | | gb:yh88b01.s1 Soares placenta Nb2HP Homo | 6.08 | 3828 |
| 427157 | U51166 | Hs.173824 | thymine-DNA glycosylase | 6.08 | 2127 5331 |
| 422725 | AA315703 | Hs.199993 | ESTs, Weakly similar to ALUB_HUMAN !!!! | 6.07 | 1676 |
| 436860 | H12751 | Hs.5327 | PRO1914 protein | 6.06 | 3012 |
| 426108 | AA622037 | Hs.166468 | programmed cell death 5 | 6.06 | 2028 |
| 419298 | AA853479 | Hs.89890 | pyruvate carboxylase | 6.05 | 1299 |
| 443695 | AW204099 | | ESTs, Weakly similar to AF126780 1 retin | 6.05 | 3541 |
| 438146 | Z36842 | Hs.57548 | ESTs | 6.05 | 3121 |
| 445786 | AW629819 | Hs.144502 | hypothetical protein FLJ22055 | 6.05 | 3697 |
| 442108 | AW452649 | Hs.166314 | ESTs | 6.04 | 3398 |
| 432374 | W68815 | Hs.301885 | Homo sapiens cDNA FLJ11346 fis, clone PL | 6.04 | 2660 |
| 414320 | U13616 | Hs.75893 | ankyrin 3, node of Ranvier (ankyrin G) | 6.03 | 4917 810 |
| 437330 | AL353944 | Hs.50115 | Homo sapiens mRNA; cDNA DKFZp761J1112 (f | 6.03 | 3058 |
| 447397 | BE247676 | Hs.18442 | E-1 enzyme | 6.03 | 3856 |
| 458814 | AI498957 | Hs.170861 | ESTs, Weakly similar to Z195_HUMAN ZINC | 6.03 | 4638 |
| 429429 | AA829725 | Hs.334437 | hypothetical protein MGC4248 | 6.03 | 2367 |
| 402736 | | | NM_024852:Homo sapiens hypothetical prot | 6.03 | 37 4706 |
| 450353 | AI244661 | Hs.103296 | ESTs, Weakly similar to S65657 alpha-1C- | 6.02 | 4132 |
| 453078 | AF053551 | Hs.31584 | metaxin 2 | 6.02 | 4373 5769 |
| 441659 | BE564162 | Hs.250820 | hypothetical protein FLJ14827 | 6.01 | 3366 |
| 439696 | W95298 | Hs.171882 | ESTs | 6.01 | 3230 |
| 417878 | U90916 | Hs.82845 | Homo sapiens cDNA:FLJ21930 fis, clone H | 6.00 | 1133 |
| 420956 | AA351584 | Hs.100543 | Homo sapiens clone 24505 mRNA sequence | 5.99 | 1468 |
| 445652 | AL117473 | Hs.13036 | DKFZP727A071 protein | 5.98 | 3690 5657 |
| 430300 | U60805 | Hs.238648 | oncostatin M receptor | 5.98 | 2465 5441 |
| 430935 | AW072916 | | zinc finger protein 131 (clone pHZ-10) | 5.98 | 2521 |
| 415276 | U88666 | Hs.78353 | SFRS protein kinase 2 | 5.97 | 4945 922 |
| 448369 | AW268962 | Hs.111335 | ESTs | 5.97 | 3961 |
| 431049 | AA846576 | Hs.103267 | hypothetical protein FLJ22548 similar to | 5.96 | 2527 |
| 417233 | W25005 | Hs.24395 | small inducible cytokine subfamily B (Cy | 5.96 | 1073 |
| 446882 | H13878 | Hs.165195 | Homo sapiens cDNA FLJ14237 fis, clone NT | 5.96 | 3800 |
| 422912 | AW405973 | Hs.11637 | ESTs | 5.96 | 1692 |
| 423134 | AJ012582 | Hs.124161 | hyperpolarization activated cyclic nucle | 5.96 | 1720 5170 |
| 429505 | AW820035 | Hs.278679 | a disintegrin and metalloproteinase doma | 5.95 | 2383 |
| 441623 | AA315805 | | desmoglein 2 | 5.94 | 3362 |
| 409407 | AW967370 | Hs.342655 | Homo sapiens cDNA FLJ13289 fis, clone OV | 5.94 | 367 |
| 408089 | H59799 | Hs.42644 | thioredoxin-like | 5.94 | 233 |
| 407110 | AA018042 | Hs.252085 | Prader-Willi/Angelman syndrome-5 | 5.93 | 125 |
| 452466 | N84635 | Hs.29664 | hypothetical protein DKFZp564B052 | 5.93 | 4312 |
| 450314 | AA574309 | Hs.283402 | TCR eta | 5.92 | 4128 |
| 452620 | AA436504 | Hs.119286 | ESTs | 5.92 | 4330 |
| 440325 | NM_003812 | Hs.7164 | a disintegrin and metalloproteinase doma | 5.91 | 3281 5606 |
| 407604 | AW191962 | | collagen, type VIII, alpha 2 | 5.91 | 172 |
| 449103 | T24968 | Hs.23038 | HSPC071 protein | 5.91 | 4036 |
| 416990 | AF124145 | Hs.80731 | autocrine motility factor receptor | 5.90 | 1049 4975 |
| 414844 | AA296874 | Hs.77494 | deoxyguanosine kinase | 5.90 | 884 |
| 430454 | AW469011 | Hs.105635 | ESTs | 5.90 | 2487 |
| 437915 | AI637993 | Hs.202312 | Homo sapiens clone N11 NTera2D1 teratoca | 5.89 | 3105 |
| 448939 | BE267795 | Hs.22595 | hypothetical protein FLJ10637 | 5.88 | 4021 |
| 426216 | N77630 | Hs.13895 | Homo sapiens cDNA FLJ11654 fis, clone HE | 5.88 | 2040 |
| 432329 | NM_002962 | Hs.2960 | S100 calcium-binding protein A5 | 5.88 | 2655 5506 |
| 452994 | AW962597 | Hs.31305 | KIAA1547 protein | 5.88 | 4363 |
| 441790 | AW294909 | Hs.132208 | ESTs | 5.86 | 3372 |
| 444099 | D87432 | Hs.10315 | solute carrier family 7 (cationic amino | 5.86 | 3575 5636 |
| 427658 | H61387 | Hs.30868 | nogo receptor | 5.86 | 2175 |
| 401866 | | | Target Exon | 5.86 | |
| 438510 | AL080220 | Hs.6285 | DKFZP586P0123 protein | 5.85 | 3143 5595 |
| 446813 | AA971436 | Hs.16218 | KIAA0903 protein | 5.85 | 3791 |
| 458748 | AI381530 | | gb:te76d07.x1 Soares_NFL_T_GBC_S1 Homo s | 5.85 | 4635 |
| 425523 | AB007948 | Hs.158244 | KIAA0479 protein | 5.85 | 1973 5268 |
| 428832 | AA578229 | Hs.324239 | ESTs, Moderately similar to ZN91_HUMAN Z | 5.85 | 2308 |
| 431846 | BE019924 | Hs.271580 | uroplakin 1B | 5.85 | 2605 |
| 422363 | T55979 | Hs.115474 | replication factor C (activator 1) 3 (38 | 5.84 | 1636 |
| 418322 | AA284166 | Hs.84113 | cyclin-dependent kinase inhibitor 3 (CDK | 5.84 | 1184 |
| 426158 | NM_001982 | Hs.199067 | v-erb-b2 avian erythroblastic leukemia v | 5.83 | 2033 5294 |
| 448122 | AW665656 | Hs.173187 | ESTs | 5.83 | 3946 |
| 436045 | AB037723 | Hs.5028 | DKFZP564O0423 protein | 5.83 | 2959 5574 |
| 442045 | C05768 | Hs.8078 | Homo sapiens clone FBD3 Cri-du-chat crit | 5.82 | 3390 |
| 409129 | AW296699 | Hs.103521 | serine arginine-rich pre-mRNA splicing f | 5.82 | 337 |
| 443162 | T49951 | Hs.9029 | DKFZP434G032 protein | 5.80 | 3500 |
| 448690 | AK001304 | Hs.21771 | Wolf-Hirschhorn syndrome candidate 2 | 5.80 | 3998 |
| 433017 | Y15067 | Hs.279914 | zinc finger protein 232 | 5.79 | 2722 5523 |
| 430168 | AW968343 | Hs.145582 | DKFZP434I1735 protein | 5.79 | 2449 |
| 449444 | AW818436 | | solute carrier family 16 (monocarboxylic | 5.79 | 4062 |
| 435798 | BE395289 | Hs.12720 | eIF4E-transporter | 5.78 | 2944 |
| 408411 | C15118 | Hs.322482 | hypothetical protein DKFZp566J2046 | 5.78 | 265 |
| 416179 | R19015 | Hs.79067 | MAD (mothers against decapentaplegic, Dr | 5.77 | 979 |
| 453902 | BE502341 | Hs.3402 | ESTs | 5.77 | 4449 |
| 422939 | AW394055 | Hs.98427 | ESTs, Weakly similar to I38022 hypotheti | 5.77 | 1695 |
| 434094 | AA305599 | Hs.238205 | hypothetical protein PRO2013 | 5.76 | 2814 |
| 437834 | AA769294 | | gb:nz36g03.s1 NCI_CGAP_GCB1 Homo sapiens | 5.76 | 3096 |
| 450516 | AA902656 | Hs.21943 | NIF3 (Ngg1 interacting factor 3, S.pombe | 5.76 | 4141 |
| 440043 | BE277457 | Hs.30661 | hypothetical protein MGC4606 | 5.75 | 3256 |
| 418816 | T29621 | Hs.88778 | carbonyl reductase 1 | 5.74 | 1243 |
| 437672 | AW748265 | Hs.5741 | flavohemoprotein b5? | 5.74 | 3080 |
| 433029 | NM_014322 | Hs.279926 | opsin 3 (encephalopsin) | 5.74 | 2726 5524 |
| 413585 | AI133452 | Hs.75431 | fibrinogen, gamma polypeptide | 5.73 | 737 |
| 413435 | X51405 | Hs.75360 | carboxypeptidase E | 5.73 | 4900 721 |
| 430044 | AA464510 | Hs.152812 | ESTs | 5.73 | 2439 |
| 439559 | AW364675 | Hs.173921 | ESTs, Weakly similar to 2109260A B cell | 5.73 | 3215 |
| 424130 | AL050136 | Hs.140945 | Homo sapiens mRNA; cDNA DKFZp586L141 (fr | 5.73 | 1806 |
| 441962 | AW972542 | Hs.289008 | Homo sapiens cDNA: FLJ21814 fis, clone H | 5.71 | 3384 |
| 433002 | AF048730 | Hs.279906 | cyclin T1 | 5.71 | 2720 5522 |
| 425463 | AK000740 | Hs.157986 | molybdenum cofactor sulfurase | 5.71 | 1968 5265 |
| 432728 | NM_006979 | Hs.278721 | HLA class II region expressed gene KE4 | 5.71 | 2694 5512 |
| 451351 | AW058261 | Hs.321435 | ESTs, Weakly similar to ALU1_HUMAN ALU S | 5.70 | 4214 |
| 404568 | | | NM_022071*:Homo sapiens hypothetical pro | 5.70 | 4723 56 |
| 419926 | AW900992 | Hs.93796 | DKFZP586D2223 protein | 5.70 | 1371 |
| 428654 | NM_012091 | Hs.188661 | adenosine deaminase, tRNA-specific 1 | 5.69 | 2280 5370 |
| 440351 | AF030933 | Hs.7179 | RAD1 (S. pombe) homolog | 5.68 | 3285 5607 |
| 448479 | H96115 | Hs.21293 | UDP-N-acteylglucosamine pyrophosphorylas | 5.67 | 3974 |
| 456141 | AI751357 | Hs.288741 | Homo sapiens cDNA: FLJ22256 fis, clone H | 5.66 | 4529 |
| 419539 | AF070590 | Hs.90869 | Homo sapiens clones 24622 and 24623 mRNA | 5.65 | 1328 |
| 409717 | AW452871 | Hs.56043 | CGI-115 protein | 5.64 | 400 |
| 408633 | AW963372 | Hs.46677 | PRO2000 protein | 5.64 | 286 |
| 409235 | AA188827 | Hs.7988 | ESTs, Weakly similar to I38022 hypotheti | 5.63 | 354 |
| 439580 | AF086401 | Hs.293847 | ESTs, Moderately similar to S65657 alpha | 5.63 | 3220 |
| 453935 | AI633770 | Hs.42572 | ESTs | 5.63 | 4453 |
| 420187 | AK001714 | Hs.95744 | hypothetical protein similar to ankyrin | 5.63 | 1393 5067 |
| 453271 | AA903424 | Hs.6786 | ESTs | 5.63 | 4390 |
| 424364 | AW383226 | Hs.163834 | ESTs, Weakly similar to G01763 atrophin- | 5.62 | 1835 |
| 415791 | H09366 | Hs.78853 | uracil-DNA glycosylase | 5.62 | 953 |
| 415701 | NM_003878 | Hs.78619 | gamma-glutamyl hydrolase (conjugase, fol | 5.62 | 4952 945 |
| 424723 | BE409813 | Hs.152337 | protein arginine N-methyltransferase 3(h | 5.61 | 1878 |
| 407930 | AA045847 | Hs.188361 | Homo sapiens cDNA FLJ12807 fis, clone NT | 5.60 | 215 |
| 408670 | AF160967 | Hs.46784 | potassium large conductance calcium-acti | 5.60 | 291 4803 |
| 447495 | AW401864 | Hs.18720 | programmed cell death 8 (apoptosis-induc | 5.59 | 3868 |
| 426318 | AA375125 | Hs.147112 | Homo sapiens cDNA:FLJ22322 fis, clone H | 5.59 | 2051 |
| 431448 | AL137517 | Hs.306201 | hypothetical protein DKFZp564O1278 | 5.58 | 2555 5476 |
| 452461 | N78223 | Hs.108106 | transcription factor | 5.58 | 4311 |
| 430598 | AK001764 | Hs.247112 | hypothetical protein FLJ10902 | 5.56 | 2499 5459 |
| 414343 | AL036166 | Hs.75914 | coated vesicle membrane protein | 5.56 | 814 |
| 433627 | AF078866 | Hs.284296 | Homo sapiens cDNA:FLJ22993 fis, clone K | 5.56 | 2774 5538 |
| 448045 | AJ297436 | Hs.20166 | prostate stem cell antigen | 5.55 | 3937 5706 |
| 428428 | AL037544 | Hs.184298 | cyclin-dependent kinase 7 (homolog of Xe | 5.55 | 2255 |
| 416883 | AW140128 | Hs.184902 | ESTs | 5.55 | 1033 |
| 420126 | NM_016255 | Hs.95260 | Autosomal Highly Conserved Protein | 5.55 | 1387 5064 |
| 408784 | AW971350 | Hs.63386 | ESTs | 5.55 | 307 |
| 417059 | AL037672 | Hs.81071 | extracellular matrix protein 1 | 5.55 | 1059 |
| 416782 | L35035 | Hs.79886 | ribose 5-phosphate isomerase A (ribose 5 | 5.53 | 1021 |
| 442366 | AA115629 | Hs.118531 | ESTs | 5.53 | 3425 |
| 429609 | AF002246 | Hs.210863 | cell adhesion molecule with homology to | 5.53 | 2394 5414 |
| 414612 | BE274552 | Hs.76578 | protein inhibitor of activated STAT3 | 5.53 | 854 |
| 419354 | M62839 | Hs.1252 | apolipoprotein H (beta-2-glycoprotein I) | 5.52 | 1303 5044 |
| 426252 | BE176980 | Hs.28917 | ESTs | 5.51 | 2044 |
| 437435 | AA249439 | Hs.27027 | hypothetical protein DKFZp762H1311 | 5.51 | 3065 |
| 435770 | AA699991 | Hs.348162 | gb:zi69a09.s1 Soares_fetal_liver_spleen_{—} | 5.51 | 2942 |
| 420230 | AL034344 | Hs.284186 | forkhead box C1 | 5.51 | 1398 5069 |
| 430539 | AK001489 | | ADP-ribosylation factor-like 1 | 5.50 | 2495 |
| 431899 | AA521381 | Hs.187726 | ESTs | 5.49 | 2614 |
| 455510 | AA422029 | Hs.143640 | ESTs, Weakly similar to hyperpolarizatio | 5.49 | 4512 |
| 451622 | AW139587 | Hs.30579 | Homo sapiens cDNA:FLJ23070 fis, clone L | 5.49 | 4236 |
| 448789 | BE539108 | Hs.22051 | hypothetical protein MGC15548 | 5.49 | 4007 |
| 434067 | H18913 | Hs.124023 | Homo sapiens cDNA FLJ14218 fis, clone NT | 5.48 | 2812 |
| 429687 | AI675749 | Hs.211608 | nucleoporin 153 kD | 5.48 | 2406 |
| 428816 | AA004986 | Hs.193852 | ATP-binding cassette, sub-family C (CFTR | 5.47 | 2303 |
| 442767 | AI017208 | Hs.131149 | ESTs | 5.45 | 3467 |
| 451259 | NM_006052 | Hs.26146 | Down syndrome critical region gene 3 | 5.45 | 4205 5740 |
| 453985 | N44545 | Hs.251865 | ESTs | 5.45 | 4457 |
| 452167 | N75238 | Hs.13075 | Homo sapiens cDNA:FLJ23013 fis, clone L | 5.45 | 4278 |
| 414341 | D80004 | Hs.75909 | KIAA0182 protein | 5.44 | 4919 813 |
| 452518 | AA280722 | Hs.24758 | ESTs, Weakly similar to I38022 hypotheti | 5.44 | 4321 |
| 438182 | AW342140 | Hs.182545 | ESTs,Weakly similar to ALU1_HUMAN ALU S | 5.43 | 3124 |
| 430057 | AW450303 | Hs.2534 | bone morphogenetic protein receptor, typ | 5.43 | 2441 |
| 433675 | AW977653 | Hs.75319 | ribonucleotide reductase M2 polypeptide | 5.43 | 2779 |
| 413476 | U25849 | Hs.75393 | acid phosphatase 1, soluble | 5.43 | 4902 726 |
| 430508 | AI015435 | Hs.104637 | ESTs | 5.42 | 2490 |
| 437204 | AL110216 | | ESTs, Weakly similar to I55214 salivary | 5.42 | 3043 |
| 430335 | D80007 | Hs.239499 | KIAA0185 protein | 5.41 | 2473 5448 |
| 446825 | BE266822 | Hs.344097 | filamin A, alpha (actin-binding protein- | 5.41 | 3792 |
| 409190 | AU076536 | Hs.50984 | sarcoma amplified sequence | 5.40 | 349 |
| 440282 | BE262386 | | clones 23667 and 23775 zinc finger prote | 5.40 | 3277 |
| 426860 | U04953 | Hs.172801 | isoleucine-tRNA synthetase | 5.40 | 2107 5325 |
| 414987 | AA524394 | Hs.294022 | hypothetical protein FLJ14950 | 5.39 | 898 |
| 451101 | N22587 | Hs.270134 | Homo sapiens cDNA FLJ11752 fis, clone HE | 5.38 | 4192 |
| 422611 | AA158177 | Hs.118722 | fucosyltransferase 8 (alpha (1,6) fucosy | 5.37 | 1662 |
| 440193 | AW902312 | Hs.7037 | Homo sapiens clone 24923 mRNA sequence | 5.37 | 3270 |
| 451593 | AF151879 | Hs.26706 | CGI-121 protein | 5.37 | 4231 5746 |
| 429503 | AA394183 | Hs.204166 | ESTs | 5.37 | 2381 |
| 427087 | BE073913 | Hs.173515 | uncharacterized hypothalamus protein HT0 | 5.36 | 2125 |
| 401403 | | | Target Exon | 5.36 | |
| 417731 | D26018 | Hs.82502 | polymerase (DNA directed), delta 3 | 5.35 | 1118 4993 |
| 444735 | BE019923 | Hs.243122 | hypothetical protein FLJ13057 similar to | 5.35 | 3623 |
| 417615 | BE548641 | Hs.82314 | hypoxanthine phosphoribosyltransferase 1 | 5.35 | 1107 |
| 416111 | AA033813 | Hs.79018 | chromatin assembly factor 1, subunit A ( | 5.34 | 972 |
| 408831 | AF090114 | Hs.48433 | endocrine regulator | 5.34 | 310 4807 |
| 413333 | M74028 | Hs.75297 | fibroblast growth factor 1 (acidic) | 5.34 | 712 |
| 416959 | D28459 | Hs.80612 | ubiquitin-conjugating enzyme E2A (RAD6 h | 5.33 | 1043 4973 |
| 457001 | J03258 | Hs.2062 | vitamin D (1,25-dihydroxyvitamin D3) re | 5.32 | 4574 5808 |
| 450251 | BE080483 | | gb:QV1-BT0630-280200-086-a05 BT0630 Homo | 5.32 | 4118 |
| 436961 | AW375974 | Hs.156704 | ESTs | 5.30 | 3023 |
| 452092 | BE245374 | Hs.27842 | hypothetical protein FLJ11210 | 5.30 | 4268 |
| 439680 | AW245741 | Hs.58461 | ESTs, Weakly similar to A35659 krueppel- | 5.30 | 3229 |
| 441865 | AA384726 | Hs.5722 | hypothetical protein FLJ23316 | 5.30 | 3376 |
| 443015 | R33261 | Hs.6614 | ESTs, Weakly similar to A43932 mucin 2 p | 5.29 | 3487 |
| 433388 | AI432672 | Hs.288539 | hypothetical protein FLJ22191 | 5.29 | 2759 |
| 421180 | BE410992 | Hs.258730 | heme-regulated initiation factor 2-alpha | 5.29 | 1486 |
| 410935 | BE067395 | Hs.66881 | dynein, cytoplasmic, intermediate polype | 5.28 | 521 |
| 427846 | AW499770 | Hs.180948 | KIAA0729 protein | 5.28 | 2200 |
| 437659 | AB007944 | Hs.5737 | KIAA0475 gene product | 5.28 | 3079 5589 |
| 442711 | AF151073 | Hs.8645 | hypothetical protein | 5.28 | 3460 5625 |
| 431197 | AL038596 | Hs.250745 | polymerase (RNA) III (DNA directed) (62 k | 5.27 | 2539 |
| 451945 | BE504055 | Hs.211420 | ESTs | 5.27 | 4258 |
| 402812 | | | NM_004930*:Homo sapiens capping protein | 5.26 | 39 4708 |
| 402802 | | | NM_001397:Homo sapiens endothelin conver | 5.26 | 38 4707 |
| 428417 | AK001699 | Hs.184227 | F-box only protein 21 | 5.26 | 2253 |
| 451468 | AW503398 | Hs.293663 | ESTs, Moderately similar to I38022 hypot | 5.25 | 4224 |
| 422975 | AA347720 | Hs.122669 | KIAA0264 protein | 5.25 | 1704 |
| 424395 | AA165082 | Hs.146388 | microtubule-associated protein 7 | 5.24 | 1839 |
| 434938 | AW500718 | Hs.8115 | Homo sapiens, clone MGC:16169, mRNA, com | 5.24 | 2880 |
| 458621 | AI221741 | Hs.117777 | ESTs | 5.24 | 4630 |
| 449209 | BE616830 | Hs.294145 | ESTs | 5.24 | 4046 |
| 443837 | AI984625 | Hs.9884 | spindle pole body protein | 5.23 | 3559 |
| 421977 | W94197 | Hs.110165 | ribosomal protein L26 homolog | 5.23 | 1588 |
| 433561 | BE540937 | Hs.20104 | hypothetical protein FLJ00052 | 5.22 | 2769 |
| 448555 | AI536697 | Hs.159863 | ESTs | 5.22 | 3984 |
| 428479 | Y00272 | Hs.334562 | cell division cycle 2, G1 to S and G2 to | 5.22 | 2264 5363 |
| 422072 | AB018255 | Hs.111138 | KIAA0712 gene product | 5.22 | 1597 5133 |
| 418250 | U29926 | Hs.83918 | adenosine monophosphate deaminase (isofo | 5.22 | 1173 5006 |
| 431771 | AW239175 | Hs.2853 | poly(rC)-binding protein 1 | 5.22 | 2597 |
| 410079 | U94362 | Hs.58589 | glycogenin 2 | 5.22 | 441 4837 |
| 449810 | AB008681 | Hs.23994 | activin A receptor, type IIB | 5.21 | 4085 5723 |
| 427735 | AA916785 | Hs.180610 | splicing factor proline/glutamine rich ( | 5.21 | 2192 |
| 424179 | F30712 | Hs.334573 | Homo sapiens, clone IMAGE:4285740, mRNA | 5.20 | 1812 |
| 448275 | BE514434 | Hs.20830 | kinesin-like 2 | 5.20 | 3955 |
| 421875 | AA299607 | Hs.98969 | ESTs | 5.20 | 1574 |
| 448770 | AA326683 | Hs.21992 | likely ortholog of mouse variant polyade | 5.19 | 4005 |
| 418248 | NM_005000 | Hs.83916 | NM_005000*:Homo sapiens NADH dehydrogena | 5.19 | 1172 5005 |
| 444563 | N57057 | Hs.284163 | ANKHZN protein | 5.19 | 3608 |
| 441181 | AA416925 | | peptidylprolyl isomerase (cyclophilin)-l | 5.19 | 3337 |
| 415025 | AW207091 | Hs.72307 | ESTs | 5.19 | 902 |
| 453496 | AA442103 | Hs.33084 | solute carrier family 2 (facilitated glu | 5.18 | 4413 |
| 400205 | | | NM_006265*:Homo sapiens RAD21 (S. pombe) | 5.18 | 1 4680 |
| 447881 | BE620886 | | GCN1 (general control of amino-acid synt | 5.17 | 3917 |
| 450052 | AI681298 | Hs.236524 | ESTs | 5.15 | 4100 |
| 452221 | C21322 | Hs.288057 | hypothetical protein FLJ22242 | 5.15 | 4282 |
| 404996 | | | Target Exon | 5.15 | |
| 432375 | BE536069 | Hs.2962 | S100 calcium-binding protein p | 5.15 | 2661 |
| 423493 | AI815965 | Hs.129683 | ubiquitin-conjugating enzyme E2D 1 (homo | 5.14 | 1753 |
| 452110 | T47667 | Hs.28005 | Homo sapiens cDNA FLJ11309 fis, clone PL | 5.14 | 4272 |
| 407804 | AF228603 | Hs.39957 | pleckstrin 2 (mouse) homolog | 5.14 | 197 4783 |
| 437258 | AL041243 | Hs.174104 | ESTs | 5.14 | 3050 |
| 447064 | AB002350 | Hs.17262 | KIAA0352 gene product | 5.14 | 3816 5680 |
| 448439 | BE613082 | Hs.28229 | ARG99 protein | 5.14 | 3970 |
| 420344 | BE463721 | Hs.97101 | putative G protein-coupled receptor | 5.13 | 1415 |
| 420440 | NM_002407 | Hs.97644 | mammaglobin 2 | 5.13 | 1422 5076 |
| 422616 | BE300330 | Hs.118725 | selenophosphate synthetase 2 | 5.13 | 1663 |
| 427254 | AL121523 | Hs.97774 | ESTs | 5.12 | 2135 |
| 451321 | AW593532 | Hs.124135 | Homo sapiens cDNA FLJ13051 fis, clone NT | 5.12 | 4210 |
| 449048 | Z45051 | Hs.22920 | similar to S68401 (cattle) glucose induc | 5.12 | 4031 |
| 428781 | AF164799 | Hs.193384 | putatative 28 kDa protein | 5.12 | 2297 5376 |
| 426230 | AA367019 | Hs.241395 | protease, serine, 1 (trypsin 1) | 5.11 | 2042 |
| 425649 | U30930 | Hs.158540 | UDP glycosyltransferase 8 (UDP-galactose | 5.11 | 1982 5273 |
| 440100 | BE382685 | Hs.158549 | ESTs, Weakly similar to T2D3_HUMAN TRANS | 5.11 | 3263 |
| 433037 | NM_014158 | Hs.279938 | HSPC067 protein | 5.10 | 2727 5525 |
| 441224 | AU076964 | Hs.7753 | calumenin | 5.10 | 3338 |
| 420734 | AW972872 | Hs.293736 | ESTs | 5.10 | 1447 |
| 419355 | AA428520 | Hs.90061 | progesterone binding protein | 5.09 | 1304 |
| 425673 | R70318 | Hs.339730 | ESTs | 5.09 | 1985 |
| 400583 | | | Target Exon | 5.09 | |
| 452737 | AK001680 | Hs.30488 | DKFZP434F091 protein | 5.09 | 4340 5762 |
| 421928 | AF013758 | Hs.109643 | polyadenylate binding protein-interactin | 5.09 | 1580 5129 |
| 439609 | AW971945 | Hs.293236 | ESTs | 5.08 | 3225 |
| 400277 | | | Eos Control | 5.08 | |
| 448100 | AV655272 | Hs.20252 | novel Ras family protein | 5.07 | 3941 |
| 445309 | AL157474 | Hs.12504 | likely ortholog of mouse Arkadia | 5.07 | 3667 5654 |
| 428180 | AI129767 | Hs.182874 | guanine nucleotide binding protein (G pr | 5.07 | 2226 |
| 412574 | BE410731 | Hs.74050 | follicular lymphoma variant translocatio | 5.07 | 648 |
| 444385 | BE278964 | Hs.11085 | CGI-111 protein | 5.06 | 3594 |
| 422095 | AI868872 | Hs.282804 | hypothetical protein FLJ22704 | 5.06 | 1603 |
| 435905 | AW997484 | Hs.5003 | KIAA0456 protein | 5.06 | 2951 |
| 417378 | R57256 | Hs.82037 | TATA box binding protein (TBP)-associate | 5.06 | 1088 |
| 453295 | W56819 | | ESTs, Weakly similar to 138022 hypotheti | 5.06 | 4391 |
| 422938 | NM _001809 | Hs.1594 | centromere protein A (17 kD) | 5.06 | 1694 5162 |
| 408363 | NM_003389 | Hs.44396 | coronin, actin-binding protein, 2A | 5.06 | 260 4794 |
| 424438 | AA340724 | Hs.271912 | ESTs, Weakly similar to A42442 integrin | 5.05 | 1845 |
| 435468 | AW362803 | Hs.166271 | ESTs | 5.05 | 2916 |
| 402053 | | | C11001722*:gi\|11436283\|ref\|XP_006959.1\| | 5.04 | |
| 419341 | N71463 | Hs.118888 | ESTs, Weakly similar to ALU1_HUMAN ALU S | 5.04 | 1302 |
| 400880 | | | NM_000611:Homo sapiens CD59 antigen p18 | 5.04 | 23 4694 |
| 428728 | NM_016625 | Hs.191381 | hypothetical protein | 5.04 | 2288 5373 |
| 429048 | AI372949 | Hs.44241 | Homo sapiens cDNA:FLJ21447 fis, clone C | 5.04 | 2329 |
| 428690 | AI948490 | Hs.98765 | ESTs | 5.04 | 2281 |
| 452256 | AK000933 | Hs.28661 | Homo sapiens cDNA FLJ10071 fis, clone HE | 5.04 | 4289 |
| 445495 | BE622641 | Hs.38489 | ESTs, Weakly similar to 138022 hypotheti | 5.03 | 3678 |
| 423554 | M90516 | Hs.1674 | glutamine-fructose-6-phosphate transamin | 5.03 | 1758 5182 |
| 436643 | AA757626 | Hs.10941 | ESTs, Weakly similar to IPP1_HUMAN PROTE | 5.03 | 2999 |
| 420522 | AW957137 | Hs.98541 | hypothetical protein | 5.03 | 1427 |
| 436995 | AI160015 | Hs.125489 | ESTs | 5.02 | 3026 |
| 421077 | AK000061 | Hs.101590 | hypothetical protein | 5.02 | 1479 5093 |
| 411412 | AJ001388 | Hs.69997 | zinc finger protein 238 | 5.02 | 4864 554 |
| 419551 | AW582256 | Hs.91011 | anterior gradient 2 (Xenepus laevis) hom | 5.02 | 1330 |
| 413687 | AI522318 | Hs.103819 | ESTs | 5.02 | 747 |
| 442313 | BE388898 | Hs.8215 | hypothetical protein FLJ11307 | 5.01 | 3413 |
| 408705 | AA312135 | Hs.46967 | HSPCO34 protein | 5.00 | 295 |
| 440660 | AI300101 | Hs.131886 | Homo sapiens cDNA: FLJ22113 fis, clone H | 5.00 | 3310 |
| 448920 | AW408009 | Hs.22580 | alkylglycerone phosphate synthase | 5.00 | 4020 |
| 439574 | AI469788 | | ESTs | 5.00 | 3219 |
| 437739 | AW579216 | Hs.264610 | ESTs, Moderately similar to Ibd1 [H. sapi | 4.99 | 3084 |
| 414405 | AI362533 | | KIAA0306 protein | 4.99 | 822 |
| 418661 | NM_001949 | Hs.1189 | E2F transcription factor 3 | 4.99 | 1222 5022 |
| 429966 | BE081342 | Hs.283037 | HSPC039 protein | 4.98 | 2431 |
| 408000 | L11690 | Hs.198689 | bullous pemphigoid antigen 1 (230/240 kD) | 4.98 | 223 4786 |
| 427209 | H06509 | Hs.92423 | KIAA1566 protein | 4.98 | 2132 |
| 421227 | R78581 | Hs.266308 | mosaic serine protease | 4.98 | 1492 |
| 451989 | AF169797 | Hs.27413 | adaptor protein containing pH domain, PT | 4.98 | 4260 5752 |
| 442328 | AI952430 | Hs.150614 | ESTs, Weakly similar to ALU4_HUMAN ALU S | 4.97 | 3419 |
| 422385 | BE549407 | Hs.115823 | ribonuclease P, 40 kD subunit | 4.97 | 1637 |
| 429617 | X89984 | Hs.211563 | B-cell CLL/lymphoma 7A | 4.97 | 2397 5417 |
| 434369 | AI650363 | Hs.116462 | ESTs | 4.97 | 2835 |
| 452144 | AA032197 | Hs.102558 | Homo sapiens, clone MGC:5352, mRNA, comp | 4.97 | 4275 |
| 436823 | AW749865 | Hs.117077 | ESTs, Weakly similar to I38022 hypotheti | 4.97 | 3009 |
| 451380 | H09280 | Hs.13234 | ESTs | 4.96 | 4218 |
| 427083 | NM_006363 | Hs.173497 | Sec23 (S. cerevisiae) homolog B | 4.96 | 2124 5330 |
| 418888 | AU076801 | Hs.89436 | cadherin 17, LI cadherin (liver-intestin | 4.95 | 1255 |
| 408771 | AW732573 | Hs.47584 | potassium voltage-gated channel, delayed | 4.95 | 305 |
| 446572 | AV659151 | Hs.282961 | ESTs | 4.95 | 3765 |
| 449145 | AI632122 | Hs.198408 | ESTs | 4.95 | 4039 |
| 433819 | AW511097 | Hs.112765 | ESTs | 4.95 | 2789 |
| 424905 | NM_002497 | Hs.153704 | NIMA (never in mitosis gene a)-related k | 4.95 | 1898 5237 |
| 424163 | NM_007071 | Hs.142245 | HERV-H LTR-associating 3 | 4.94 | 1811 5201 |
| 436865 | AW880358 | Hs.339808 | hypothetical protein FLJ10120 | 4.94 | 3014 |
| 449437 | AI702038 | Hs.100057 | Homo sapiens cDNA: FLJ22902 fis, clone K | 4.94 | 4061 |
| 415558 | AA885143 | Hs.125719 | ESTs | 4.94 | 937 |
| 425683 | AB037813 | Hs.159200 | hypothetical protein DKFZp762K222 | 4.94 | 1987 5275 |
| 453891 | AB037751 | Hs.36353 | Homo sapiens mRNA full length insert cDN | 4.93 | 4446 5787 |
| 438613 | C05569 | Hs.243122 | hypothetical protein FLJ13057 similar to | 4.93 | 3153 |
| 402408 | | | NM_030920*:Homo sapiens hypothetical pro | 4.93 | 33 4703 |
| 428829 | R14050 | Hs.194051 | Homo sapiens mRNA; cDNA DKFZp566B213 (fr | 4.93 | 2306 |
| 418699 | BE539639 | Hs.173030 | ESTs, Weakly similar to ALU8_HUMAN ALU S | 4.92 | 1229 |
| 422576 | BE548555 | Hs.118554 | CGI-83 protein | 4.92 | 1659 |
| 415068 | Z19448 | Hs.131887 | ESTs, Weakly similar to T24396 hypotheti | 4.92 | 904 |
| 410275 | U85658 | Hs.61796 | transcription factor AP-2 gamma (activat | 4.92 | 462 4842 |
| 432540 | AI821517 | Hs.105866 | ESTs | 4.92 | 2678 |
| 422168 | AA586894 | Hs.112408 | S100 calcium-binding protein A7 (psorias | 4.92 | 1612 |
| 411704 | AI499220 | Hs.71573 | hypothetical protein FLJ10074 | 4.91 | 571 |
| 418803 | U50079 | Hs.88556 | histone deacetylase 1 | 4.90 | 1241 5029 |
| 401747 | | | Homo sapiens keratin 17 (KRT17) | 4.90 | |
| 437387 | AI198874 | Hs.28847 | AD026 protein | 4.90 | 3062 |
| 433929 | AI375499 | Hs.27379 | ESTs | 4.90 | 2799 |
| 411251 | R19774 | Hs.22835 | HHGP protein | 4.90 | 542 |
| 422567 | AF111178 | Hs.118407 | glypican 6 | 4.89 | 1658 5154 |
| 424956 | AW198103 | Hs.18724 | ESTs, Weakly similar to granule cell mar | 4.89 | 1902 |
| 414051 | BE244127 | | gb:TCBAP1E0661 Pediatric pre-B cell acut | 4.89 | 778 |
| 447083 | AI472124 | Hs.157757 | ESTs | 4.89 | 3820 |
| 456977 | AK000252 | Hs.169758 | hypothetical protein FLJ20245 | 4.88 | 4572 5807 |
| 446795 | AI797713 | Hs.156471 | ESTs | 4.88 | 3788 |
| 421515 | Y11339 | Hs.105352 | GalNAc alpha-2, 6-sialyltransferase I, I | 4.88 | 1532 5113 |
| 438705 | AI049624 | Hs.283390 | ESTs, Weakly similar to 2109260A B cell | 4.88 | 3160 |
| 441553 | AA281219 | Hs.121296 | ESTs | 4.87 | 3357 |
| 417715 | AW969587 | Hs.86366 | ESTs | 4.87 | 1115 |
| 442229 | AI885776 | Hs.8164 | Mulibrey nanism | 4.87 | 3406 |
| 436797 | AA731491 | Hs.334477 | hypothetical protein MGC14879 | 4.87 | 3007 |
| 442739 | NM_007274 | Hs.8679 | cytosolic acyl coenzyme A thioester hydr | 4.87 | 3464 5626 |
| 407813 | AL120247 | Hs.40109 | KIAA0872 protein | 4.87 | 200 |
| 457819 | AA057484 | Hs.35406 | FLJ20522 Hypothetical protein FLJ20522 | 4.86 | 4601 |
| 430291 | AV660345 | Hs.238126 | CGI-49 protein | 4.86 | 2462 |
| 430396 | D49742 | Hs.241363 | hyaluronan-binding protein 2 | 4.86 | 2481 5450 |
| 428330 | L22524 | Hs.2256 | matrix metalloproteinase 7 (matrilysin, | 4.85 | 2241 5354 |
| 427719 | AI393122 | Hs.134726 | ESTs | 4.85 | 2189 |
| 442660 | AW138174 | Hs.130651 | ESTs | 4.85 | 3458 |
| 435858 | AF254260 | Hs.283009 | tuftelin 1 | 4.84 | 2950 5572 |
| 443179 | AI928402 | Hs.6933 | hypothetical protein FLJ12684 | 4.84 | 3503 |
| 414591 | AI888490 | Hs.55902 | ESTs, Weakly similar to ALU8_HUMAN ALU S | 4.84 | 850 |
| 438459 | T49300 | Hs.35304 | Homo sapiens cDNA FLJ13655 flu, clone PL | 4.84 | 3140 |
| 409262 | AK000631 | Hs.52256 | hypothetical protein FLJ20624 | 4.84 | 355 4819 |
| 407198 | H91679 | | gb:yv04a07.s1 Soares fetal liver spleen | 4.84 | 135 |
| 450737 | AW007152 | Hs.63325 | transmembrane protease, serine 4 | 4.84 | 4157 |
| 421202 | AF193339 | Hs.102506 | eukaryotic translation initiation factor | 4.83 | 1489 5097 |
| 419586 | AI088485 | Hs.144759 | ESTs, Weakly similar to I38022 hypotheti | 4.83 | 1335 |
| 452459 | AI356895 | Hs.49359 | hypothetical protein DKFZp547E052 | 4.83 | 4310 |
| 413429 | BE139117 | Hs.278881 | ESTs | 4.83 | 719 |
| 443426 | AF098158 | Hs.9329 | chromosome 20 open reading frame 1 | 4.82 | 3523 5627 |
| 413431 | AW246428 | Hs.75355 | ubiquitin-conjugating enzyme E2N (homolo | 4.82 | 720 |
| 427832 | AF038362 | Hs.180930 | TBP-associated factor 172 | 4.82 | 2198 5347 |
| 430000 | AW205931 | Hs.99598 | hypothetical protein MGC5338 | 4.80 | 2435 |
| 408296 | AL117452 | Hs.44155 | DKFZP586G1517 protein | 4.80 | 252 4792 |
| 447444 | AK000318 | Hs.18616 | hypothetical protein FLJ20311 | 4.80 | 3862 5689 |
| 425387 | AB037864 | Hs.156051 | KIAA1443 protein | 4.80 | 1958 5260 |
| 447514 | AI809314 | Hs.208501 | ESTs, Weakly similar to B34087 hypotheti | 4.80 | 3872 |
| 430333 | S70114 | Hs.239489 | TIA1 cytotoxic granule-associated RNA-bi | 4.79 | 2472 5447 |
| 435092 | AL137310 | Hs.4749 | Homo sapiens mRNA; cDNA DKFZp761E13121 ( | 4.79 | 2895 5562 |
| 412537 | AL031778 | | nuclear transcription factor Y, alpha | 4.79 | 644 |
| 407204 | R41933 | Hs.140237 | ESTs, Weakly similar to ALU1_HUMAN ALU S | 4.79 | 136 |
| 417366 | BE185289 | Hs.1076 | small proline-rich protein 1B (cornifin) | 4.78 | 1087 |
| 411387 | AW842339 | Hs.130815 | hypothetical protein FLJ21870 | 4.78 | 550 |
| 422244 | Y08890 | Hs.113503 | karyopherin (importin) beta 3 | 4.78 | 1619 5143 |
| 448186 | AA262105 | Hs.4094 | Homo sapiens cDNA FLJ14208 fis, clone NT | 4.78 | 3949 |
| 422420 | U03398 | Hs.1524 | tumor necrosis factor (ligand) superfami | 4.78 | 1643 5150 |
| 421898 | AA259011 | Hs.109268 | hypothetical protein FLJ12552 | 4.78 | 1578 |
| 408349 | BE546947 | Hs.44276 | homeo box C10 | 4.78 | 258 |
| 445892 | AV655500 | Hs.93961 | Homo sapiens mRNA; cDNA DKFZp667D095 (fr | 4.78 | 3709 |
| 414108 | AI267592 | Hs.75761 | SFRS protein kinase 1 | 4.78 | 788 |
| 442961 | BE614474 | | F-box only protein 22 | 4.77 | 3484 |
| 441024 | AW081530 | Hs.268231 | ESTs | 4.77 | 3327 |
| 448552 | AW973653 | Hs.20104 | hypothetical protein FLJ00052 | 4.77 | 3983 |
| 424197 | AF096834 | Hs.142989 | germ cell specific Y-box binding protein | 4.77 | 1814 5202 |
| 414222 | AL135173 | Hs.878 | sorbitol dehydrogenase | 4.76 | 805 |
| 425769 | U72513 | Hs.159486 | Human RPL13-2 pseudogene mRNA, complete | 4.76 | 1995 |
| 425424 | NM_004954 | Hs.157199 | ELKL motif kinase | 4.75 | 1962 5263 |
| 426699 | AA383337 | Hs.121269 | ESTs | 4.75 | 2089 |
| 424085 | NM_002914 | Hs.139226 | replication factor C (activator 1) 2 (40 | 4.75 | 1803 5198 |
| 438869 | AF075009 | | gb:Homo sapiens full length insert cDNA | 4.75 | 3171 |
| 451027 | AW519204 | Hs.40808 | Homo sapiens, Similar to RIKEN cDNA 2810 | 4.74 | 4183 |
| 422684 | BE561617 | Hs.119192 | H2A histone family, member Z | 4.74 | 1673 |
| 403485 | | | C3001813*:gi\|12737279\|ref\|XP_012163.1\|k | 4.74 | |
| 447334 | AA515032 | Hs.91109 | ESTs | 4.74 | 3844 |
| 441989 | AA306207 | Hs.286241 | protein kinase, cAMP-dependent, regulato | 4.74 | 3385 |
| 432673 | AB028859 | Hs.278605 | DnaJ (Hsp40) homolog, subfamily B, membe | 4.73 | 2689 5510 |
| 448362 | AA641767 | Hs.21015 | hypothetical protein DKFZp564L0864 simil | 4.73 | 3960 |
| 453199 | AI336266 | Hs.32353 | mitogen-activated protein kinase kinase | 4.73 | 4384 |
| 450149 | AW969781 | Hs.132863 | Zic family member 2 (odd-paired Drosophi | 4.73 | 4106 |
| 419502 | AU076704 | | fibrinogen, A alpha polypeptide | 4.72 | 1321 |
| 442787 | W93048 | Hs.250723 | hypothetical protein MGC2747 | 4.72 | 3469 |
| 447764 | NM_003776 | Hs.19500 | nuclear localization signal deleted in v | 4.72 | 3902 5700 |
| 425118 | AU076611 | Hs.154672 | methylene tetrahydrofolate dehydrogenase | 4.71 | 1923 |
| 438714 | AA814859 | | ESTs | 4.71 | 3161 |
| 409463 | AI458165 | Hs.17296 | hypothetical protein MGC2376 | 4.71 | 375 |
| 429610 | AB024937 | Hs.211092 | LUNX protein; PLUNC (palate lung and nas | 4.70 | 2395 5415 |
| 411031 | W37943 | Hs.34892 | KIAA1323 protein | 4.70 | 530 |
| 409445 | AW341217 | Hs.14139 | ESTs, Weakly similar to JC5314 CDC28/cdc | 4.70 | 373 |
| 446019 | AI362520 | | histone deacetylase 3 | 4.70 | 3719 |
| 440074 | AA863045 | Hs.10669 | ESTs, Weakly similar to T00050 hypotheti | 4.70 | 3259 |
| 415989 | AI267700 | | ESTs | 4.70 | 963 |
| 415992 | C05837 | Hs.145807 | hypothetical protein FLJ13593 | 4.70 | 964 |
| 439772 | AL365406 | Hs.10268 | Homo sapiens mRNA full length insert cDN | 4.69 | 3234 |
| 414462 | BE622743 | Hs.301064 | arfaptin 1 | 4.69 | 831 |
| 433626 | AF078859 | Hs.86347 | hypothetical protein | 4.69 | 2773 5537 |
| 430512 | AF182294 | Hs.241578 | U6 snRNA-associated Sm-like protein LSm8 | 4.68 | 2491 5453 |
| 446269 | AW263155 | Hs.14559 | hypothetical protein FLJ10540 | 4.68 | 3741 |
| 412828 | AL133396 | Hs.74621 | prion protein (p27-30) (Creutzfeld-Jakob | 4.68 | 4889 675 |
| 409342 | AU077058 | Hs.54089 | BRCA1 associated RING domain 1 | 4.68 | 362 |
| 407907 | AI752235 | Hs.41270 | procollagen-lysine, 2-oxoglutarate 5-dio | 4.68 | 214 |
| 416586 | D44643 | Hs.14144 | secreted modular calcium-binding protein | 4.67 | 1005 |
| 442592 | BE566623 | Hs.29899 | ESTs, Weakly similar to G02075 transcrip | 4.67 | 3450 |
| 432378 | AI493046 | Hs.146133 | ESTs | 4.67 | 2662 |
| 423551 | AA327598 | Hs.89633 | ESTs | 4.67 | 1757 |
| 453753 | BE252983 | Hs.35086 | ubiquitin specific protease 1 | 4.67 | 4429 |
| 452834 | AI638627 | Hs.105685 | KIAA1688 protein | 4.66 | 4352 |
| 445733 | BE295568 | Hs.13225 | UDP-Gal:betaGlcNAc beta 1,4-galactosylt | 4.66 | 3696 |
| 430027 | AB023197 | Hs.227743 | KIAA0980 protein | 4.65 | 2438 5433 |
| 403817 | | | NM_015271:Homo sapiens tripartite motif- | 4.65 | 4716 48 |
| 453742 | AB037744 | Hs.34892 | KIAA1323 protein | 4.65 | 4427 5781 |
| 417665 | AW852858 | Hs.22862 | ESTs | 4.65 | 1110 |
| 412970 | AB026436 | Hs.177534 | dual specificity phosphatase 10 | 4.65 | 4890 687 |
| 441377 | BE218239 | Hs.202656 | ESTs | 4.65 | 3349 |
| 438898 | AI819863 | Hs.106243 | ESTs | 4.65 | 3177 |
| 428386 | R17298 | Hs.295923 | seven in absentia (Drosophila) homolog 1 | 4.65 | 2248 |
| 449644 | AW960707 | Hs.148324 | ESTs | 4.84 | 4072 |
| 424511 | BE300512 | Hs.193557 | ESTs, Moderately similar to ALU7_HUMAN A | 4.64 | 1854 |
| 415632 | U67085 | Hs.78524 | TcD37 homolog | 4.63 | 4950 939 |
| 427779 | AA906997 | Hs.180780 | TERA protein | 4.63 | 2195 |
| 422486 | BE514492 | Hs.117487 | gene near HD on 4p16.3 with homology to | 4.63 | 1648 |
| 406627 | T64904 | Hs.163780 | ESTs | 4.63 | 80 |
| 418529 | AW005695 | Hs.250897 | TRK-fused gene | 4.63 | 1212 |
| 453785 | AI368236 | Hs.283732 | ESTs, Moderately similar to ALU1_HUMAN A | 4.62 | 4432 |
| 416902 | AA375634 | Hs.288974 | hypothetical protein FLJ12528 | 4.61 | 1035 |
| 436505 | AJ277841 | Hs.120963 | ELG protein | 4.60 | 2990 5580 |
| 438378 | AW970529 | Hs.86434 | hypothetical protein FLJ21816 | 4.60 | 3136 |
| 442760 | BE075297 | Hs.6614 | ESTs, Weakly similar to A43932 mucin 2 p | 4.60 | 3466 |
| 432339 | AW411259 | | ESTs | 4.60 | 2656 |
| 452567 | D87120 | Hs.29882 | predicted osteoblast protein | 4.59 | 4326 5759 |
| 447850 | AB018298 | Hs.19822 | SEC24 (S. cerevisiae) related gene famil | 4.59 | 3914 5702 |
| 443171 | BE281128 | Hs.9030 | TONDU | 4.59 | 3501 |
| 448854 | AW245617 | Hs.77703 | hypothetical protein FLJ11506 | 4.59 | 4314 |
| 423681 | AB023215 | Hs.131525 | Homo sapiens mRNA; cDNA DKFZp434E199 (fr | 4.58 | 1766 5184 |
| 420750 | AW190215 | Hs.62348 | hypothetical protein FLJ11753 | 4.58 | 1449 |
| 439866 | AA280717 | Hs.6727 | Ras-GTPase activating protein SH3 domain | 4.58 | 3240 |
| 446719 | W39500 | Hs.301872 | hypothetical protein MGC4840 | 4.58 | 3779 |
| 429588 | AI080271 | | ESTs | 4.58 | 2391 |
| 417171 | BE613486 | Hs.81412 | lipin 1 | 4.57 | 1067 |
| 452279 | AA286844 | | hypothetical protein FLJ13164 | 4.57 | 4293 |
| 408483 | AA464836 | Hs.291079 | ESTs, Weakly similar to T27173 hypotheti | 4.57 | 271 |
| 430569 | AF241254 | Hs.178098 | angiotensin I converting enzyme (peptidy | 4.57 | 2496 5456 |
| 440773 | AA352702 | Hs.37747 | Homo sapiens, Similar to RIKEN cDNA 2700 | 4.56 | 3318 |
| 418422 | AW440068 | Hs.59425 | hypothetical protein FLJ23323 | 4.56 | 1199 |
| 447519 | U46258 | Hs.339665 | ESTs | 4.56 | 3873 |
| 434834 | AF156774 | Hs.324020 | 1-acylglycerol-3-phosphate O-acyltransfe | 4.56 | 2872 5557 |
| 412560 | R24601 | | CCR4-NOT transcription complex, subunit | 4.55 | 645 |
| 449571 | AW016812 | Hs.200266 | ESTs | 4.55 | 4069 |
| 432502 | NM_014641 | Hs.277585 | KIAA0170 gene product | 4.55 | 2674 5508 |
| 422630 | AA313606 | Hs.13809 | hypothetical protein FLJ10648 | 4.55 | 1664 |
| 423673 | BE003054 | Hs.1695 | matrix metalloproteinase 12 (macrophage | 4.55 | 1765 |
| 443228 | W24781 | Hs.293798 | KIAA1710 protein | 4.55 | 3510 |
| 427283 | AL119796 | Hs.174185 | ectonucleotide pyrophosphatase/phosphodi | 4.55 | 2137 |
| 408761 | AA057264 | Hs.238936 | ESTs, Weakly similar to (defline not ava | 4.54 | 304 |
| 408743 | AL110246 | Hs.47367 | KIAA1785 protein | 4.54 | 301 |
| 448822 | BE149845 | Hs.289038 | hypothetical protein MGC4126 | 4.54 | 4010 |
| 443450 | N66045 | Hs.133529 | ESTs | 4.54 | 3524 |
| 441021 | AW578716 | Hs.7644 | H1 histone family, member 2 | 4.53 | 3326 |
| 446111 | W56338 | Hs.13880 | CGI-143 protein | 4.53 | 3725 |
| 436679 | AI127483 | Hs.120451 | ESTs, Weakly similar to unnamed protein | 4.53 | 3002 |
| 433586 | T85301 | | gb:yd78d06.s1 Soares fetal liver spleen | 4.53 | 2770 |
| 412811 | H06382 | | ESTs | 4.52 | 673 |
| 450770 | AA019924 | Hs.28803 | ESTs | 4.52 | 4163 |
| 420439 | AW270041 | | eukaryotic translation initiation factor | 4.52 | 1421 |
| 418663 | AK001100 | Hs.41690 | desmocollin 3 | 4.52 | 1223 |
| 430299 | W28673 | Hs.106747 | serine carboxypeptidase 1 precursor prot | 4.52 | 2464 |
| 422040 | AA172106 | Hs.110950 | Rag C protein | 4.52 | 1595 |
| 429504 | X99133 | Hs.204238 | lipocalin 2 (oncogene 24p3) (NGAL) | 4.51 | 2382 5411 |
| 450187 | AA736788 | Hs.78521 | KIAA1717 protein | 4.51 | 4110 |
| 443257 | AI334040 | Hs.11614 | HSPC065 protein | 4.51 | 3514 |
| 415691 | AW963979 | Hs.24723 | ESTs | 4.51 | 944 |
| 445867 | AF272663 | Hs.13405 | gephyrin | 4.51 | 3705 5660 |
| 424075 | AI807320 | Hs.227630 | RE1-silencing transcription factor | 4.51 | 1799 |
| 445165 | AV652831 | Hs.234058 | gb:AV652831 GLC Homo sapiens cDNA clone | 4.51 | 3658 |
| 421743 | T35958 | Hs.107614 | DKFZP56411171 protein | 4.51 | 1557 |
| 447805 | AW627932 | Hs.302421 | gemin4 | 4.50 | 3908 |
| 440146 | AW014231 | Hs.90790 | Homo sapiens cDNA:FLJ22930 fis, clone K | 4.50 | 3268 |
| 448610 | NM_006157 | Hs.21602 | nel (chicken)-like 1 | 4.50 | 3989 5712 |
| 432706 | NM_013230 | Hs.286124 | CD24 antigen (small cell lung carcinoma | 4.50 | 2691 5511 |
| 408882 | H12084 | Hs.31110 | ESTs, Weakly similar to MAGE-B4 [H. sapie | 4.49 | 315 |
| 450747 | AI064821 | Hs.318535 | ESTs, Highly similar to 1818357A EWS gen | 4.49 | 4159 |
| 430178 | AW449612 | Hs.152475 | 3'UTR of:achaete-scute complex (Drosoph | 4.49 | 2451 |
| 419594 | AA013051 | Hs.91417 | topoisomerase (DNA) II binding protein | 4.49 | 1339 |
| 435641 | AI147545 | Hs.114172 | ESTs | 4.49 | 2933 |
| 420005 | AW271106 | Hs.133294 | ESTs | 4.48 | 1372 |
| 402496 | | | Target Exon | 4.48 | |
| 425997 | AK000086 | Hs.165948 | hypothetical protein FLJ20079 | 4.48 | 2018 5286 |
| 420662 | R71292 | Hs.99821 | hypothetical protein FLJ14547 | 4.48 | 1442 |
| 429651 | D79248 | Hs.279870 | ESTs, Weakly similar to A46010 X-linked | 4.48 | 2401 |
| 431130 | NM_006103 | Hs.2719 | HE4; epididymis-specific, whey-acidic pr | 4.48 | 2533 5470 |
| 425177 | AF127577 | Hs.155017 | nuclear receptor interacting protein 1 | 4.47 | 1933 5250 |
| 433323 | AA805132 | Hs.159142 | ESTs | 4.47 | 2755 |
| 421437 | AW821252 | Hs.104336 | hypothetical protein | 4.47 | 1519 |
| 427969 | NM_001963 | Hs.2230 | epidermal growth factor (beta-urogastron | 4.47 | 2208 5348 |
| 420798 | W93774 | Hs.99936 | keratin 10 (epidermolytic hyperkeratosis | 4.47 | 1454 |
| 440590 | AI863446 | Hs.266308 | mosaic serine protease | 4.46 | 3301 |
| 452598 | AI831594 | | ESTs, Weakly similar to ALU7_HUMAN ALU S | 4.46 | 4328 |
| 456439 | AA251242 | Hs.103238 | ESTs | 4.46 | 4542 |
| 447574 | AF162666 | Hs.18895 | tousled-like kinase 1 | 4.46 | 3881 5694 |
| 437469 | AW753112 | Hs.15514 | hypothetical protein MGC3260 | 4.46 | 3066 |
| 410444 | W73484 | Hs.132554 | gb:zd54e04.s1 Soares_fetal_heart_NbHH19W | 4.46 | 480 |
| 452689 | F33868 | Hs.284176 | transferrin | 4.45 | 4335 |
| 447832 | AI433357 | | ESTs | 4.45 | 3911 |
| 441381 | H22195 | Hs.31874 | ESTs | 4.45 | 3351 |
| 425548 | AA890023 | Hs.1906 | prolactin receptor | 4.45 | 1978 |
| 429702 | AA456883 | Hs.79889 | ESTs | 4.45 | 2409 |
| 450669 | AL138077 | Hs.16157 | hypothetical protein FLJ12707 | 4.45 | 4146 |
| 446742 | AA232119 | Hs.16085 | putative G-protein coupled receptor | 4.45 | 3780 |
| 421814 | L12350 | Hs.108623 | thrombospondin 2 | 4.45 | 1567 5124 |
| 445160 | AI299144 | Hs.101937 | sine oculis homeobox (Drosophila) homolo | 4.44 | 3657 |
| 437763 | AA469369 | Hs.5831 | tissue inhibitor of metalloproteinase 1 | 4.44 | 3089 |
| 449343 | AI151418 | | protein phosphatase 3 (formerly 2B), cat | 4.44 | 4056 |
| 426765 | AA743603 | Hs.172108 | nucleoporin 88 kD | 4.44 | 2096 |
| 408527 | AL135018 | Hs.33074 | Homo sapiens, clone IMAGE:3606519, mRNA, | 4.44 | 276 |
| 438360 | H74149 | Hs.288193 | hypothetical protein MGC12217 | 4.43 | 3134 |
| 432840 | AK001403 | Hs.279521 | hypothetical protein FLJ20530 | 4.43 | 2704 5516 |
| 415443 | T07353 | Hs.7948 | ESTs | 4.43 | 931 |
| 429084 | AJ001443 | Hs.195614 | splicing factor 3b, subunit 3, 130 kD | 4.43 | 2332 5390 |
| 406687 | M31126 | | matrix metalloproteinase 11 (stromelysin | 4.43 | 4747 85 |
| 454417 | AI244459 | Hs.110826 | trinucleotide repeat containing 9 | 4.43 | 4480 |
| 417534 | NM_004998 | Hs.82251 | myosin IE | 4.43 | 1100 4989 |
| 421754 | N76984 | Hs.107922 | hypothetical protein | 4.42 | 1558 |
| 410054 | AL120050 | Hs.58220 | Homo sapiens cDNA:FLJ23005 fis, clone L | 4.41 | 435 |
| 450684 | AA872605 | Hs.25333 | interleukin 1 receptor, type II | 4.41 | 4148 |
| 446567 | NM_007247 | Hs.15384 | AP1 gamma subunit binding protein 1 | 4.41 | 3764 5672 |
| 428474 | AB023182 | Hs.184523 | KIAA0965 protein | 4.41 | 2263 5362 |
| 446356 | AI816736 | Hs.14896 | DHHC1 protein | 4.40 | 3747 |
| 447155 | AA100605 | Hs.121557 | ESTs, Weakly similar to DP1_HUMAN POLYPO | 4.40 | 3827 |
| 416852 | AF283776 | Hs.80285 | Homo sapiens mRNA:cDNA DKFZp586C1723 (f | 4.40 | 1030 |
| 419713 | AW968058 | Hs.92381 | nudix (nucleoside diphosphate linked moi | 4.40 | 1352 |
| 421609 | H39924 | Hs.106148 | Homo sapiens mRNA; cDNA DKFZp434G0972 (f | 4.40 | 1543 |
| 432350 | NM_005865 | Hs.274407 | protease, serine, 16 (thymus) | 4.40 | 2658 5507 |
| 451273 | NM_014811 | Hs.26163 | KIAA0649 gene product | 4.40 | 4206 5741 |
| 418757 | AI864193 | Hs.169728 | hypothetical protein FLJ13150 | 4.40 | 1236 |
| 415660 | AI909007 | Hs.78563 | ubiquitin-conjugating enzyme E2G 1 (homo | 4.40 | 943 |
| 439588 | AA838166 | Hs.174644 | hypothetical protein FLJ21669 | 4.39 | 3221 |
| 427678 | BE267756 | Hs.180312 | mitochondrial ribosomal protein S16 | 4.39 | 2180 |
| 418259 | AA215404 | | ESTs | 4.39 | 1174 |
| 450649 | NM_001429 | Hs.25272 | E1A binding protein p300 | 4.38 | 4143 5732 |
| 446783 | AW138343 | Hs.141867 | ESTs | 4.38 | 3786 |
| 401464 | | | histone deacetylase 5 | 4.38 | |
| 402855 | | | NM_001839*:Homo sapiens calponin 3, acid | 4.38 | 40 4709 |
| 448256 | BE614149 | Hs.20814 | CGI-27 protein | 4.38 | 3952 |
| 442053 | R35343 | Hs.24968 | Human DNA sequence from clone RP1-233G16 | 4.38 | 3391 |
| 435248 | AA676865 | Hs.188965 | ESTs | 4.38 | 2902 |
| 418867 | D31771 | Hs.89404 | msh (Drosophila) homeo box homolog 2 | 4.38 | 1252 5032 |
| 440603 | AL121733 | Hs.7299 | Novel human gene mapping to chomosome 1 | 4.38 | 3304 5610 |
| 423445 | NM_014324 | Hs.128749 | alpha-methylacyl-CoA racemase | 4.37 | 1746 5179 |
| 443242 | BE243910 | Hs.9082 | nucleoporin p54 | 4.37 | 3512 |
| 448360 | AL117560 | Hs.306352 | Homo sapiens mRNA; cDNA DKFZp566P2324 (f | 4.37 | 3959 |
| 431562 | AI884334 | Hs.11637 | ESTs | 4.37 | 2566 |
| 418565 | AK001529 | Hs.86149 | phosphoinositol 3-phosphate-binding prot | 4.37 | 1213 5019 |
| 416801 | X98834 | Hs.79971 | sal (Drosophila)-like 2 | 4.36 | 1024 |
| 434804 | AA649530 | | gb:ns44f05.s1 NCI_CGAP_Alv1 Homo sapiens | 4.36 | 2866 |
| 412123 | BE251328 | Hs.73291 | hypothetical protein FLJ10881 | 4.36 | 610 |
| 406081 | | | Target Exon | 4.36 | |
| 413599 | AJ006239 | Hs.75438 | quinoid dihydropteridine reductase | 4.35 | 4904 740 |
| 427712 | AI368024 | Hs.283696 | ESTs | 4.35 | 2187 |
| 423309 | BE006775 | Hs.126782 | sushi-repeat protein | 4.35 | 1736 |
| 452921 | AI694200 | | API5-like 1 | 4.35 | 4359 |
| 408908 | BE296227 | Hs.250822 | serine/threonine kinase 15 | 4.35 | 317 |
| 432148 | AW504912 | Hs.81907 | ESTs | 4.35 | 2636 |
| 434807 | AA364183 | Hs.323443 | hypothetical protein FLJ11806 | 4.35 | 2867 |
| 408056 | AA312329 | Hs.42331 | ephrin-A4 | 4.35 | 229 |
| 409132 | AJ224538 | Hs.50732 | protein kinase, AMP-activated, beta 2 no | 4.35 | 338 4813 |
| 408212 | AA297567 | Hs.43728 | hypothetical protein | 4.34 | 245 |
| 423176 | AA322771 | Hs.184864 | Homo sapiens, clone IMAGE:3162799, mRNA, | 4.34 | 1725 |
| 418372 | AA311833 | Hs.84318 | replication protein A1 (70 kD) | 4.34 | 1191 |
| 424668 | D83702 | Hs.151573 | cryptochrome 1 (photolyase-like) | 4.34 | 1869 5223 |
| 437228 | AL122099 | Hs.75066 | translin | 4.34 | 3046 |
| 428878 | AA436884 | Hs.48926 | ESTs | 4.33 | 2315 |
| 400370 | AF147075 | Hs.278686 | p53-responsive gene 3 | 4.33 | 14 |
| 439955 | AW203959 | Hs.149532 | ESTs | 4.33 | 3249 |
| 430200 | BE613337 | Hs.234896 | geminin | 4.32 | 2454 |
| 411450 | H49619 | Hs.127301 | ESTs | 4.32 | 557 |
| 426997 | BE620738 | Hs.173125 | peptidylprolyl isomerase F (cyclophilin | 4.32 | 2120 |
| 430791 | AA486293 | Hs.272068 | ESTs, Weakly similar to ALU3_HUMAN ALU S | 4.31 | 2513 |
| 438746 | AI885815 | Hs.184727 | Human melanoma-associated antigen p97 (m | 4.31 | 3164 |
| 443941 | AW016271 | Hs.134883 | ESTs | 4.31 | 3564 |
| 453323 | AF034102 | Hs.32951 | solute carrier family 29 (nucleoside tra | 4.31 | 4395 5775 |
| 439108 | AW163034 | Hs.6467 | synaptogyrin 3 | 4.31 | 3186 |
| 431214 | AA294921 | Hs.348024 | v-ral simian leukemia viral oncogene hom | 4.30 | 2541 |
| 431912 | AI660552 | | ESTs, Weakly similar to A56154 Abl subst | 4.30 | 2615 |
| 425580 | L11144 | Hs.1907 | galanin | 4.30 | 1979 5272 |
| 408161 | AW952912 | Hs.300383 | hypothetical protein MGC3032 | 4.30 | 240 |
| 409264 | NM_014937 | Hs.52463 | KIAA0966 protein | 4.30 | 356 4820 |
| 421288 | AI916563 | Hs.178499 | Homo sapiens cDNA:FLJ23117 fis, clone L | 4.30 | 1503 |
| 430526 | AF181862 | Hs.242407 | G protein-coupled receptor, family C, gr | 4.29 | 2493 5455 |
| 440426 | AI159800 | Hs.7181 | Homo sapiens cDNA FLJ13663 fis, clone PL | 4.29 | 3287 |
| 430691 | C14187 | Hs.157208 | aristaless-related homeobox protein ARX | 4.29 | 2505 |
| 455505 | AW970640 | Hs.309071 | ESTs | 4.29 | 751 |
| 413882 | AA132973 | Hs.184492 | ESTs | 4.29 | 761 |
| 405387 | | | NM_022170*:Homo sapiens Williams-Beuren | 4.29 | 4736 70 |
| 424005 | AB033041 | Hs.137507 | vang (van gogh, Drosophila)-like 2 | 4.29 | 1790 5193 |
| 420090 | AA220238 | Hs.94986 | ribonuclease P (38 kD) | 4.28 | 1383 |
| 417386 | AL037228 | Hs.82043 | D123 gene product | 4.28 | 1090 |
| 437866 | AA156781 | | metallothionein 1E (functional) | 4.28 | 3101 |
| 401558 | | | ENSP00000220478*:SECRETOGRANIN III. | 4.28 | |
| 430315 | NM_004293 | Hs.239147 | guanine deaminase | 4.28 | 2469 5444 |
| 432169 | Y00971 | Hs.2910 | phosphoribosyl pyrophosphate synthetase | 4.28 | 2637 5501 |
| 436869 | NM_014867 | Hs.5333 | KIAA0711 gene product | 4.28 | 3015 5581 |
| 410561 | BE540255 | Hs.6994 | Homo sapiens cDNA:FLJ22044 fis, clone H | 4.28 | 493 |
| 429162 | AK001250 | Hs.197642 | hypothetical protein FLJ10388 | 4.27 | 2334 5391 |
| 411968 | AI207410 | Hs.69280 | Homo sapiens, clone IMAGE:3636299, mRNA, | 4.27 | 595 |
| 422809 | AK001379 | Hs.121028 | hypothetical protein FLJ10549 | 4.27 | 1684 |
| 442619 | AA447492 | Hs.20183 | ESTs, Weakly similar to AF164793 1 prote | 4.27 | 3455 |
| 425242 | D13635 | Hs.155287 | KIAA0010 gene product | 4.27 | 1942 |
| 413413 | D82520 | | zinc finger protein 36 (KOX 18) | 4.27 | 717 |
| 416933 | BE561850 | Hs.80506 | small nuclear ribonucleoprotein polypept | 4.27 | 1040 |
| 420851 | AA281062 | Hs.29493 | hypothetical protein FLJ20142 | 4.27 | 1460 |
| 421508 | NM_004833 | Hs.105115 | absent in melanoma 2 | 4.27 | 1529 5111 |
| 446100 | AW967109 | Hs.13804 | hypothetical protein dJ462O23.2 | 4.27 | 3724 |
| 431578 | AB037759 | Hs.261587 | GCN2 elF2alpha kinase | 4.26 | 2570 5480 |
| 458187 | D56919 | Hs.265848 | myomegalin | 4.26 | 4614 |
| 425159 | NM_004341 | Hs.154868 | carbamoyl-phosphate synthetase 2, aspart | 4.26 | 1931 5249 |
| 439186 | AI697274 | Hs.105435 | GDP-mannose 4,6-dehydratase | 4.25 | 3191 |
| 408471 | NM_012317 | Hs.45231 | leucine zipper, down-regulated in cancer | 4.25 | 269 4797 |
| 451320 | AW118072 | | diacylglycerol kinase, zeta (104 kD) | 4.25 | 4209 |
| 420805 | L10333 | Hs.99947 | reticulon 1 | 4.25 | 1456 5088 |
| 424992 | AW290893 | Hs.96918 | Homo sapiens cDNA:FLJ21561 fis, clone C | 4.25 | 1905 |
| 443195 | BE148235 | Hs.193063 | Homo sapiens cDNA FLJ14201 fis, clone NT | 4.25 | 3506 |
| 458820 | BE552151 | Hs.108118 | hypothetical protein FLJ22474 | 4.25 | 4640 |
| 453455 | AA063553 | Hs.221931 | ESTs, Weakly similar to JC1087 RNA helic | 4.24 | 4408 |
| 448105 | AW591433 | Hs.298241 | Transmembrane protease, serine 3 | 4.24 | 3943 |
| 419591 | AF090900 | Hs.91393 | Homo sapiens cDNA:FLJ21887 fis, clone H | 4.24 | 1338 5054 |
| 410434 | AF051152 | Hs.63668 | toll-like receptor 2 | 4.24 | 478 4847 |
| 438186 | AA779910 | Hs.122128 | ESTs, Weakly similar to VMD2_HUMAN BESTR | 4.23 | 3125 |
| 452833 | BE559681 | Hs.30736 | KIAA0124 protein | 4.23 | 4351 |
| 450401 | AW959281 | Hs.8184 | ESTs | 4.23 | 4138 |
| 408075 | AA382881 | Hs.42409 | CGI-146 protein | 4.23 | 231 |
| 445139 | AB037848 | Hs.12365 | synaptotagmin XIII | 4.23 | 3656 5652 |
| 437158 | AW090198 | | KIAA1150 protein | 4.23 | 3039 |
| 431736 | AI912234 | Hs.3297 | ribosomal protein S27a | 4.22 | 2594 |
| 434203 | BE262677 | Hs.283558 | hypothetical protein PRO1855 | 4.22 | 2820 |
| 418182 | AW016405 | Hs.16648 | ESTs | 4.22 | 1167 |
| 428770 | AK001667 | Hs.193128 | hypothetical protein FLJ10805 | 4.22 | 2294 5374 |
| 427853 | AI569798 | Hs.98260 | ESTs | 4.22 | 2201 |
| 444151 | AW972917 | Hs.128749 | alpha-methylacyl-CoA racemase | 4.22 | 3577 |
| 424529 | M90656 | Hs.151393 | glutamate-cysteine ligase, catalytic sub | 4.22 | 1865 5221 |
| 445929 | AI089660 | Hs.323401 | dpy-30-like protein | 4.21 | 3714 |
| 452357 | AI638176 | Hs.283865 | ESTs | 4.21 | 4299 |
| 418216 | AA662240 | Hs.283099 | AF15q14 protein | 4.21 | 1171 |
| 440726 | AL050333 | Hs.306425 | DKFZP564B116 protein | 4.21 | 3315 |
| 408548 | AA055449 | Hs.63187 | ESTs, Weakly similar to ALUC_HUMAN !!!! | 4.20 | 278 |
| 418126 | T91451 | Hs.86538 | ESTs | 4.20 | 1163 |
| 434045 | AI065133 | Hs.152316 | hypothetical protein PR00971 | 4.20 | 2809 |
| 415752 | BE314524 | Hs.78776 | putative transmembrane protein | 4.20 | 948 |
| 419480 | BE536584 | Hs.122546 | hypothetical protein FLJ23017 | 4.20 | 1317 |
| 432626 | AA471098 | Hs.278544 | acetyl-Coenzyme A acetyltransferase 2 (a | 4.20 | 2687 |
| 416984 | H38765 | Hs.80706 | diaphorase (NADH/NADPH) (cytochrome b-5 | 4.19 | 1047 |
| 424720 | AA89907 | Hs.152292 | SWI/SNF related, matrix associated, acti | 4.19 | 1877 5228 |
| 429227 | AI961456 | Hs.21275 | hypothetical protein FLJ11011 | 4.19 | 2339 |
| 437250 | BE257342 | Hs.94576 | hypothetical protein MGC3062 | 4.19 | 3048 |
| 429412 | NM_006235 | Hs.2407 | POU domain, class 2, associating factor | 4.19 | 2365 5404 |
| 425867 | D60385 | Hs.12079 | calsyntenin-2 | 4.19 | 2007 |
| 438833 | BE612940 | Hs.88252 | ESTs | 4.18 | 3170 |
| 442064 | AI422867 | Hs.88594 | ESTs | 4.18 | 3392 |
| 440624 | AF017987 | Hs.7306 | secreted frizzled-related protein 1 | 4.18 | 3306 5611 |
| 415728 | X77337 | Hs.78713 | solute carrier family 25 (mitochondrial | 4.18 | 4953 946 |
| 430287 | AW182459 | Hs.125759 | ESTs, Weakly similar to LEU5_HUMAN LEUKE | 4.18 | 2460 |
| 439819 | BE246331 | Hs.98401 | Homo sapiens mRNA full length insert cDN | 4.18 | 3237 |
| 409151 | AA306105 | Hs.50785 | SEC22, vesicle trafficking protein (S. c | 4.18 | 341 |
| 400810 | | | NM_006560:Homo sapiens CUG triplet repea | 4.18 | 20 4691 |
| 447898 | AW969638 | Hs.112318 | 6.2 kd protein | 4.18 | 3921 |
| 448412 | AI219083 | Hs.42532 | ESTs, Moderately similar to ALU8_HUMAN A | 4.18 | 3967 |
| 411761 | AI733848 | Hs.71935 | putative zinc finger protein from EUROIM | 4.18 | 574 |
| 418506 | AA084248 | Hs.85339 | Unknown protein for MGC:29643 (formerly | 4.17 | 1208 |
| 438054 | AA776626 | Hs.169309 | ESTs | 4.17 | 3115 |
| 407756 | AA116021 | Hs.38260 | ubiquitin specific protease 18 | 4.16 | 191 |
| 442073 | AW973443 | Hs.8086 | RNA (guanine-7-) methyltransferase | 4.16 | 3396 |
| 426227 | U67058 | Hs.154299 | Human proteinase activated receptor-2 mR | 4.16 | 2041 |
| 435513 | AW404075 | Hs.42785 | DC11 protein | 4.16 | 2922 |
| 458513 | AI138322 | Hs.154894 | ESTs | 4.16 | 4627 |
| 419373 | NM_003244 | Hs.90077 | TG-interacting factor (TALE family homeo | 4.16 | 1307 5045 |
| 452862 | AW378065 | Hs.8687 | ADAMTS2 (a disintegrin-like and metallo | 4.15 | 4354 |
| 428465 | AW970976 | Hs.293653 | ESTs | 4.15 | 2260 |
| 417266 | AW978515 | Hs.131915 | KIAA0863 protein | 4.15 | 1075 |
| 415786 | AW419196 | Hs.257924 | hypothetical protein FLJ13782 | 4.15 | 951 |
| 432878 | BE386490 | Hs.279663 | Pirin | 4.15 | 2707 |
| 412122 | AW852707 | | G-rich RNA sequence binding factor 1 | 4.15 | 609 |
| 425116 | AU076686 | Hs.154668 | KIAA0391 gene product | 4.13 | 1922 |
| 433023 | AW864793 | | thrombospondin 1 | 4.13 | 2725 |
| 421808 | AK000157 | Hs.108502 | hypothetical protein FLJ20150 | 4.13 | 1565 5122 |
| 432229 | AW290976 | Hs.143587 | ESTs | 4.13 | 2646 |
| 411145 | BE439553 | Hs.12329 | Homo sapiens, clone IMAGE:4098694, mRNA, | 4.13 | 537 |
| 442607 | AA507576 | Hs.288361 | Homo sapiens cDNA:FLJ22696 fis, clone H | 4.12 | 3452 |
| 453751 | R36762 | Hs.101282 | Homo sapiens cDNA:FLJ21238 fis, clone C | 4.12 | 4428 |
| 437108 | AA434054 | Hs.80624 | hypothetical protein MGC2560 | 4.12 | 3034 |
| 426784 | U03749 | Hs.172216 | chromogranin A (parathyroid secretory pr | 4.11 | 2098 5319 |
| 447541 | AK000288 | Hs.18800 | hypothetical protein FLJ20281 | 4.11 | 3877 5691 |
| 433077 | AA314262 | Hs.289008 | YDD19 protein | 4.11 | 2732 |
| 416640 | BE262478 | Hs.79404 | neuron-specific protein | 4.11 | 1009 |
| 407634 | AW016569 | Hs.136414 | UDP-GlcNAc:betaGal beta-1,3-N-acetylgluc | 4.11 | 174 |
| 441128 | AA570256 | | ESTs, Weakly similar to T23273 hypotheti | 4.10 | 3334 |
| 408031 | AA081395 | Hs.42173 | Homo sapiens cDNA FLJ10366 fis, clone NT | 4.10 | 225 |
| 407839 | AA045144 | Hs.161566 | ESTs | 4.10 | 205 |
| 425001 | U55184 | Hs.154145 | hypothetical protein FLJ1585 | 4.10 | 1908 5241 |
| 449188 | AW072939 | Hs.347187 | myotubularin related protein 1 | 4.10 | 4043 |
| 434941 | AW073202 | Hs.334825 | Homo sapiens cDNA FLJ14752 fis, clone NT | 4.09 | 2881 |
| 409439 | AW390511 | Hs.288862 | Homo sapiens cDNA: FLJ21260 fis, clone C | 4.09 | 371 |
| 424243 | AI949359 | Hs.143600 | ESTs, Highly similar to cis Golgi-locali | 4.09 | 1818 |
| 439606 | W79123 | Hs.58561 | G protein-coupled receptor 87 | 4.08 | 3224 |
| 407999 | AI126271 | Hs.49433 | ESTs, Weakly similar to YZ28_HUMAN HYPOT | 4.08 | 222 |
| 414962 | AF273304 | Hs.235376 | XPMC2 protein | 4.08 | 4936 896 |
| 452512 | AW363486 | Hs.337635 | ESTs | 4.08 | 4319 |
| 429024 | AI652297 | Hs.119302 | complement-c1q tumor necrosis factor-rel | 4.07 | 2326 |
| 434288 | AW189075 | Hs.116265 | fibrillin3 | 4.07 | 2826 |
| 438472 | AW974907 | Hs.86228 | TRIAD3 protein | 4.07 | 3142 |
| 449543 | AF070632 | Hs.23729 | Homo sapiens clone 24405 mRNA sequence | 4.07 | 4066 |
| 439541 | AW970853 | Hs.336214 | ESTs | 4.06 | 3213 |
| 435803 | Z44194 | Hs.4994 | transducer of ERBB2, 2 | 4.06 | 2946 |
| 429250 | H56585 | Hs.198308 | tryptophan rich basic protein | 4.06 | 2343 |
| 437994 | U92012 | Hs.251659 | ESTs, Weakly similar to mariner transpos | 4.06 | 3114 |
| 434725 | AK000796 | Hs.4104 | hypothetical protein | 4.06 | 2863 5553 |
| 440006 | AK000517 | Hs.6844 | NALP2 protein; PYRIN-Containing APAF1-ti | 4.05 | 3252 5601 |
| 424259 | AK001776 | Hs.143954 | hypothetical protein FLJ10914 | 4.05 | 1822 5207 |
| 426572 | AB037783 | Hs.170623 | hypothetical protein FLJ11183 | 4.05 | 2079 5310 |
| 434517 | AA635690 | Hs.337251 | hypothetical protein MGC2487 | 4.05 | 2845 |
| 425397 | J04088 | Hs.156346 | topoisomerase (DNA)∥alpha (170 kD) | 4.05 | 1959 5261 |
| 434033 | AI631749 | Hs.156616 | ESTs, Weakly similar to alternatively sp | 4.05 | 2807 |
| 431341 | AA307211 | Hs.251531 | proteasome (prosome, macropain) subunit, | 4.05 | 2549 |
| 427699 | AW965076 | Hs.180378 | hypothetical protein 669 | 4.04 | 2184 |
| 422765 | AW409701 | Hs.1578 | baculoviral IAP repeat-containing 5 (sur | 4.04 | 1680 |
| 452408 | AA306477 | Hs.29379 | hypothetical protein FLJ10687 | 4.04 | 4304 |
| 412708 | R26830 | Hs.106137 | ESTs, Weakly similar to CGHU7L collagen | 4.04 | 661 |
| 424232 | AB015982 | Hs.143460 | protein kinase C, nu | 4.04 | 1816 5204 |
| 432945 | AL043683 | Hs.8173 | hypothetical protein FLJ10803 | 4.04 | 2715 |
| 430132 | AA204686 | Hs.234149 | hypothetical protein FLJ20647 | 4.04 | 2446 |
| 431585 | BE242803 | Hs.262823 | hypothetical protein FLJ10326 | 4.04 | 2572 |
| 410660 | AI061118 | Hs.65328 | Fanconi anemia, complementation group F | 4.04 | 502 |
| 414013 | AA766605 | Hs.47099 | hypothetical protein FLJ21212 | 4.04 | 774 |
| 414696 | AF002020 | Hs.76918 | Niemann-Pick disease, type C1 | 4.04 | 4924 864 |
| 419638 | N46504 | Hs.91747 | profilin 2 | 4.04 | 1341 |
| 446487 | AA195526 | Hs.44625 | Rad50-interacting protein 1 | 4.03 | 3754 |
| 419749 | X73608 | Hs.93029 | sparc/osteonectin, cwcv and kazal-like d | 4.03 | 1357 5057 |
| 444864 | AW965446 | Hs.81907 | ESTs, Weakly similar to T26501 hypotheti | 4.03 | 3635 |
| 445921 | AW015211 | Hs.146181 | ESTs | 4.03 | 3712 |
| 422005 | BE266556 | Hs.110702 | Homo sapiens mRNA; cDNA DKFZp761E212 (fr | 4.03 | 1592 |
| 406922 | S70284 | Hs.119597 | gb:stearoyl-CoA desaturase [human, adipo | 4.03 | 109 4755 |
| 433149 | BE257672 | Hs.42949 | hypothetical protein HES6 | 4.02 | 2740 |
| 421182 | AA284655 | Hs.104480 | ESTs | 4.02 | 1487 |
| 417691 | AU076610 | Hs.82399 | low density lipoprotein receptor defect | 4.02 | 1112 |
| 451253 | H48299 | Hs.26126 | claudin 10 | 4.02 | 4204 |
| 446950 | AA305800 | Hs.5672 | hypothetical protein AF140225 | 4.01 | 3806 |
| 435020 | AW505076 | Hs.301855 | DiGeorge syndrome critical region gene 8 | 4.01 | 2888 |
| 432278 | AL137506 | Hs.274256 | hypothetical protein FLJ23563 | 4.01 | 2650 5504 |
| 452066 | AA772149 | Hs.16979 | ESTs, Weakly similar to A43932 mucin 2 p | 4.01 | 4267 |
| 444700 | NM_003645 | Hs.11729 | fatty-acid-Coenzyme A ligase, very long- | 4.01 | 3620 5642 |
| 417777 | AI823763 | Hs.7055 | ESTs, Weakly similar to I78885 serine/th | 4.00 | 1122 |
| 441647 | AA534210 | Hs.285280 | Homo sapiens cDNA:FLJ22096 fis, clone H | 4.00 | 3365 |
| 453063 | W47196 | Hs.166172 | aryl hydrocarbon receptor nuclear transl | 4.00 | 4371 |
| 432834 | F06459 | Hs.289113 | cytochrome b5 reductase 1 (B5R.1) | 4.00 | 2703 |
| 419457 | AA243146 | Hs.209334 | ESTs, Moderately similar to S23A_HUMAN P | 4.00 | 1315 |
| 452253 | AA928891 | Hs.28608 | Homo sapiens cDNA:FLJ22115 fis, clone H | 4.00 | 4288 |

Pkey: Unique Eos probeset identifier number
ExAccn: Exemplar Accession number, Genbank accession number
UniGeneID: UniGene number
UniGene Title: UniGene gene title
R1: 90th percentile of lung metastases to the brain Als divided by the 90th percentile of normal lung Als, where the minimum in the numerator and denominator was set to 20.
SEQ ID NO(s): SEQ ID number(s) for nucleic acid and protein sequences associated with table entry.

TABLE 6B

| Pkey | CAT number | Accession |
|---|---|---|
| 449230 | 6182_6 | BC006329 AK026224 BG105365 AI472084 AW074277 AI743908 BM309990 BI850432 AI094365 AI539568 AW779999 |
| | | AI261365 AA988975 AI436272 AI559886 BI710742 AA988508 AA036678 AI867147 AW518513 AI620019 BE645777 AI801919 AW205320 AI457722 BF061996 |
| | | BF061798 AA233854 AA232795 AA516294 AA830561 R74220 BG768337 AW392972 BE764979 BE257665 |
| 429228 | 215430_1 | BG676155 BM009591 AI479075 AI025794 AI017967 AA448270 BE466812 AA853422 AI392649 BG952034 AA513384 BF840124 BE714620 AW969605 AI553633 |
| 447329 | 9170_1 | BC001573 AK074197 AW967044 AW360965 AW612048 AI765501 AA112034 BI859391 BI334670 W63553 AV706135 AW024203 BI194441 AV655141 AA316041 BE280583 |
| | | BG428780 BE267060 AV648926 AA317168 BI222294 BG437688 BE709273 AI951240 AI611162 AW970792 H89338 BE543309 AL036038 R26941 |
| | | BI765176 AA209487 AA111975 AA086463 AW192209 AW025245 BF511894 AW264490 AW014985 AA947336 F15843 BI335083 |
| | | AA563626 BE546579 AW470009 AA083693 BE090517 BF970294 BG427898 BE541527 |
| 428342 | 6712_1 | AK056315 AI015524 AA724079 BI713619 AI377728 AW293682 AI928140 AI092404 AI085630 AA731340 BM469629 AW968804 AA425658 AI356361 AI923640 AW070509 |
| | | AA769094 BF446026 AW118719 AI332765 AW500888 AW576556 AI859571 AW499664 AW614573 AW629495 AW505314 W74704 AI521500 AL042095 AA609309 |
| | | AA761319 AI381489 H45700 AA761333 AW265424 AA909524 AA635311 AA649040 AI392620 Z40708 AI985564 AW263513 AA913892 AI693486 AW263502 |
| | | AI806164 AW291137 BI061872 BI059498 AA134476 AW084888 AA036967 AW370823 T55263 BI002756 AA489664 BF827261 W74741 BF963166 |
| 412446 | 63467_1 | BC021735 AI669212 AL120184 AI769949 BE701002 BE184363 BE819031 BG702238 BF090049 BF963318 BF961912 BF943013 |
| | | AA934514 AA151245 BF960659 AA987907 Z41449 BF908059 BF908053 BF908049 BE699424 BF908060 BF962832 BF952020 BF963134 |
| | | BI035538 BF908052 BF908057 BF090026 BF943158 AI632924 BF512340 BF952021 BF960776 BF943437 BF942847 AI768015 F09778 |
| | | F04816 F02721 AA102645 AI633838 AA617929 BF947001 BI035448 BE935876 AW890837 AW898604 BF957405 BF963433 BG704815 |
| 412636 | 1438_1 | M77830 NM_004415 AF139065 BG681115 BG740377 BI712964 BG000656 AA128470 BI438324 H27408 BE931630 BE167165 AW370827 |
| | | AW370813 J05211 BG698865 BG740734 BG680618 BG739778 BI765807 BM353403 BM353248 AW177784 AW205789 AW951576 |
| | | AW848592 BE182164 BF149266 BE940187 BI060445 BI060444 BF350983 BE720095 BE720069 BE715154 BE082584 BE082576 |
| | | BE004047 AA857316 BI039774 BE713818 BE713548 AW170253 BE160433 BI039775 AW886475 BM462504 BE931734 BF149264 |
| | | AA340777 BF381183 BG621737 AU127260 AW364859 BF993352 BG223489 BE819009 BF381184 BE715956 R58704 AA852212 |
| | | AW366566 BI090358 BF087707 BE819046 BE819005 AA377127 BE073467 BE819069 BE819048 BI036306 BG990973 BI040954 |
| | | BF919911 AU140155 AI951766 AI434518 AW804674 BF752969 BE837009 BE925826 BF149265 AW995615 BE814264 BI039782 |
| | | AU140407 BE144243 BE709863 BF985642 BE001923 BF933510 AW265328 BG436319 BE182166 AW365175 AW847688 BE818280 |
| | | AW177933 BF873679 AW178000 BE082526 BF476866 BF086994 BF592276 BE082507 BE082514 BE082505 BF873693 AW068840 |
| | | AW847678 BF804153 AW365157 BE813930 BE002030 AW365153 BE184941 BF749421 BE184920 BF839562 BE184933 BF842254 |
| | | BE698470 BE931048 BF999889 BF368816 BE184924 BE159646 BE714632 BE184948 BG986845 AA131128 AA099891 W39488 C04715 |
| | | BF096124 BE865341 AW799304 AL603116 BE149760 BE705967 BE705966 BE705968 AW848723 AW376699 AW376817 AW376697 |
| | | BG005097 BF751115 BE696084 AW848371 AW376782 AW848789 AW849074 AW361413 BF927725 BF094211 AW997139 BE865474 |
| | | BE185187 BE156621 BE715089 BE713297 BE713298 BE179915 AW799309 BF872345 BF088676 BE705939 AW752599 BG005197 |
| | | BF350086 BE715196 BE715155 BF752396 BF093817 BF831190 BF752409 BE006561 BG959922 BF094833 BF094748 BF094583 |
| | | AW377699 AW607238 BE082519 AW377700 BF349467 AI190590 AI554403 AI392926 AU158477 BI467252 AU159919 AI760816 BF082516 |
| | | AI439101 AA451923 AI340326 AI590975 BI791553 AI700963 AI142882 AA039975 AA946936 AA644381 BM314884 AA702424 AI417612 |
| | | AW190555 AI220573 AI304772 AI270345 AI627383 AA552300 AI911702 AW166807 AI346078 W95070 AA149191 AA026864 AI830049 |
| | | AW780435 AI078449 AI819984 AI858282 BI468588 AI860584 AI025932 AA026047 AA703232 AA658154 AA515500 AW192085 AA918281 |
| | | T77861 AI927207 AI205263 BF082491 AW021347 AI568096 BE939862 AA088866 D12062 AA056527 AA782109 W19287 W02156 |
| | | AW150038 AA022701 T87181 H44405 AI910434 BF082513 AI494069 AI270027 AI635878 AA128330 BG681425 BE706078 R20904 |
| | | BG680059 BG676647 BF764409 AA026654 AV745530 BI762796 BG287391 AW798780 BE706045 BE926470 AW799118 BF087996 |
| | | BE002273 AW879451 AI571075 BE067786 AV721320 AI022862 N29754 C03378 N84767 AA131077 H30146 BE714290 AI686869 |
| | | AI568892 AI915596 AW105614 AI887258 AI538577 BE926474 BE067737 BG319486 AA247685 AW798883 AW103521 BF989173 |
| | | AW860878 BE939707 BE185750 BE714064 BE713903 BE713868 BE713763 BG950164 BE713810 AW365151 BG955489 BE005272 |
| | | BF915937 AW365148 AI905927 BF992780 AW853812 BG954443 BI770853 BG679406 BG740832 BG681087 BG698430 AA455100 |
| | | T87267 BE696209 BE696210 BI089483 BE006273 BE872225 AW391912 BE925515 BG677012 BG741970 AA026480 BE705999 |
| | | BG677157 BE009090 BG681378 BE712291 BG961498 BG678984 BI040941 AA337270 AW384371 AW847442 BI058659 BE813665 |
| | | W95048 W25458 AW177786 AA025851 BE931733 BF154837 BG949393 BE714441 AW996245 BE711801 AI284090 BE064323 BE719390 |
| | | BE940148 BG991212 BF375714 BF349522 BG996267 T48793 BI013292 BE001925 AW365156 AW365154 AW606653 BF763109 |
| | | BE931637 BE167181 BE713879 BF354008 BF678726 H90899 AW365145 W38382 AI498487 |
| 449349 | 852_3 | BG679689 AW856638 BM016319 BE327123 AW772128 BE693337 BE938262 BG013928 BF892774 BF894765 BF892588 BF890995 |
| | | BE155056 BE153569 AI934407 BE672538 AW204203 AA778306 BE502974 AI718504 AA778312 AW008224 AW299732 AI911561 |
| | | AI867457 AI521962 AI640173 AI823832 AI288888 BE467960 AI934441 AA483527 AW612103 AI802712 AW342106 AI580299 AW083293 |
| | | AI700874 AW469932 AI583726 AW302136 BE327360 AW614404 C02300 AA934834 F29737 AI262050 AA934619 AA535965 BF196507 |
| | | AA393480 BF086615 AI825386 AA009773 BI333272 T93614 AW770207 BF766665 T64641 W92713 R94110 T89897 BF086603 T93659 |
| | | AA001207 BE539257 BE541430 BE160783 BE155304 BE155454 BF891405 BF762818 |
| 451807 | 17758_2 | BM479185 AL552795 AL577722 BF038888 BM127617 BF510346 AW450652 AA865478 AW449519 BM127314 AI806539 AW449522 AA993634 AI827626 AA904788 |
| 446873 | 15731_1 | BC016767 Z30093 NM_001516 BI753904 AL555563 AL598882 BG706296 BG723901 AL600911 AW949486 AL602675 AL600169 AL600111 AI373329 |
| | | BF149149 AU099109 AL578036 AA410532 AA521208 BI018894 BI333338 AW269273 N79350 AW954400 AI760772 AA765916 AW300373 AW628851 AW505118 AA506136 |
| | | AW779373 AI675026 BE674159 AI241579 N51870 AI632936 AI796901 AI620962 AA876979 AA886127 AA934448 H58689 AA373345 N78420 AA282406 N98953 N22575 |
| | | N62381 AA805377 AV718946 AI421009 D20327 AW518433 AI554439 BI027833 BG613441 BG573433 N66669 N62701 |
| 450377 | 12109_1 | AB033091 AL520743 BE811813 N53332 N99716 AL561910 AA280655 BE710392 AV705100 AW293978 AW444556 AA281459 AI679751 |
| | | AI873695 BG700891 BI553517 R80518 BG779771 BG534451 AA479402 AW961580 BF061430 AI857643 AI768486 AW512118 AA479302 |
| | | AW770384 AW072470 AI041596 AI049699 AW592865 AA976261 BE879747 BG114119 AW183811 AA909938 BF571621 BF350794 |
| | | BF351375 BE925699 AL050294 BC010371 BF982270 AL042656 BF095732 AW812618 BF095731 BG212397 BF678765 BI038602 |
| | | BG388664 AW675337 BG289398 BE939598 AW805570 AA527097 AW150540 AI693720 AA743364 AI915793 N48185 AI573107 AA043474 |
| | | AI351615 AI969490 AI910763 R50866 AI699181 N73808 H08164 |
| 451752 | 10408_5 | AB032997 AI141678 AW978722 BE467119 AI761408 BF727385 AW237035 AI934521 BF436248 AI479668 Z40632 AA832081 AW295901 BF057835 BE465977 |
| | | AI621269 BE465983 BF756369 N74056 AI817896 AA716567 AA934774 H62600 H09497 BF943762 BE395335 BE883333 |
| 400247 | 2764_1 | BC022339 BC009610 BC010537 X79805 NM_006713 U12979 BM467814 BM450743 AU132951 AU137129 BG493425 AV758819 |
| | | BG708412 BG705885 BG702217 AV716638 BG777009 BI545689 BI552153 BM476712 BG770858 BG527656 BG528277 BG391388 |
| | | AV716861 BI602926 BG290073 BI667399 BM451469 BI667173 BI602139 BG532171 BI669216 BI544727 BG721852 AV716503 AV701327 |
| | | BM090738 BI492000 AI308856 BI544904 AL599813 AV715829 AV716505 AV714587 AV717902 BF668072 AV716385 BI461927 BM090954 |
| | | AV717826 BG503676 AV647719 BG501392 BG428433 BE895629 BM313117 AW021050 BG435032 BM152910 AA313503 AA872377 |
| | | BG574714 AV712054 AV732696 AA252476 AV712759 AL599643 BE790872 BG654930 W73337 AW675377 AV760376 AV725139 |
| | | AV716379 AA887165 BE830003 AW023796 AL599291 AI902948 BG944042 F00781 AA352483 BG217897 N33888 AW581924 BG654730 |
| | | D31410 AA353088 D31288 AA295029 H95170 BE935104 AU139980 BG772963 BG776470 BG532512 BG105449 BI545421 AV715456 |
| | | AW386083 BG699714 AL535832 AL514940 BG190861 BG210593 AW999254 H95138 AA353863 BE764809 N50375 BE091363 BG701255 |
| | | BI860846 BI832485 BG168150 BG028647 BE546301 BG900321 BI909737 BG702363 BG614141 BG611137 BG700121 BF031492 N85802 |
| | | AV715940 N51590 BG993478 BE172016 AW893622 |
| 444172 | 49300_2 | BF526827 AA513594 AL515291 AV648373 AV648176 AA916789 BF002906 AW469960 BE466943 AI367749 AI559715 BF431260 AA937968 AI422252 AI288937 |
| | | BF962778 AA909144 BF960004 BE671534 AI271719 BF925335 BE669504 BF433431 BF924838 AI218062 AW960577 AV722716 BI859067 BF944964 |
| | | BE147740 BF938993 |
| 414883 | 8371_2 | AF274943 BG494894 AI719075 AA908783 AI935150 AI422691 AA910644 AA583187 BM272167 AI828996 AA527373 AW972459 AI831360 |
| | | AA772418 AI033892 AA100926 AU154749 AI459432 AI423513 AI094597 AA740817 AI991988 AI090262 AI312104 BI256707 AA459522 |
| | | AA416871 AI075239 AI339996 AA701623 AI139549 AI336880 AA633648 AI989380 AI362835 AA399239 AI146955 BF514270 N92892 |
| | | AI348243 AI278887 AA459292 AI494230 BF507531 AI492600 AA962596 AW613002 AA293140 AA235549 BF108854 AA954344 N49682 |
| | | AI457100 AW589407 AW300758 BE220715 BE220698 BE569091 BM009647 BF900351 AI537692 AI203723 AI857576 AA584410 |
| | | AW371667 BM172363 |
| 412652 | 18858_2 | AI801777 BE677762 AW008210 AW009441 BE350994 BE207949 AI091475 AI802774 AI827533 AI075363 AI659979 AA687855 AI078125 |
| | | AI090285 AA670058 AA602411 AA683472 AI436058 AA612826 AI038932 BG057726 AI167355 BF449023 AI289476 AW074381 BF972912 |
| | | AI991780 AA889119 AI537472 Z39730 AI868953 AI192337 BE812978 BE812939 AA115248 H99006 AI915784 F08973 T16748 D20468 |
| | | AA609899 BF081234 AA115336 BE812876 BE812972 BE812982 BE813006 BE813019 R43883 BE812981 |
| 419631 | 2743_1 | BC022323 AF204171 NM_022361 BM264431 BE670789 AW188117 AI025298 AA861832 H84897 AI382294 AA662874 AW993380 |
| | | BE813742 H84368 AI188074 N20482 H84369 |
| 422150 | 782_1 | BI086421 BF059136 AW003898 BF446659 AI632891 AI628067 AI703179 AI961149 BF111022 AW614154 BE674215 AA687350 AA779426 BF591963 AW243344 AI356530 |
| | | AI492508 AI694049 AI090422 AA465307 AI273387 BE674625 AW271971 AA969153 AW468593 AA984014 AI817491 AA970258 AI914450 AI018697 AW577591 |
| | | AW577616 AA382101 AW954455 AI867266 AI707995 AI337384 BF208406 BF037100 BF223433 BF195517 BE673984 BF224124 BE813387 BG036579 BI553906 |
| | | AA304995 AW361269 AW754160 AW361276 AW361271 AI867118 AW805555 AW361284 AW954458 AW958551 BG681507 H79011 AW205696 AW134957 AA747667 |
| | | AW753296 BF939060 AW958549 BF910827 BG573750 BG168639 |
| 418304 | 1093209_1 | BE883520 BI057842 AA215702 AA215703 AA368006 BE006876 BE066555 |
| 437810 | 6139_1 | NM_016617 AF208844 Z70222 AL515797 BI666750 BI602334 BG717532 AL554429 BI545991 AW847749 BG393649 AV757080 AI373461 |
| | | AA315780 AA301659 AI393910 AI762600 AA321522 AA331540 AF305827 BG924826 AI720715 AI079860 AA677539 AA707143 AW196875 |
| | | AI269926 AI929790 BE246362 AI753224 AI687566 AI022486 AA101081 BM150935 AI591063 AI566293 AW189841 AW515306 AW028380 |
| | | AA766135 AI084005 W02557 AI018470 AA972561 AI371710 AA954779 AA746269 AA747909 AW020207 BI491245 H89948 AI041765 |
| | | AV652999 AA889510 AW243602 AA628783 AI866249 AA736845 AL515796 BF970987 AW604215 AW604214 R53127 AA937309 |
| | | AA815183 AA976070 AA736764 AW473513 BF591731 H88829 AL566858 BI088846 R73732 BF679743 BG113547 BF243416 |
| 412452 | 71091_1 | BE796667 BF330981 BE394193 Z45547 BG490525 F35734 AA130708 AA577072 AA446587 AA215665 AA978209 BG740729 BG746810 BE298184 AI356291 AI671975 |
| | | AI818924 AV715722 AI078381 BI142391 AI201085 AI198283 AI077572 AI694848 AW016425 BM456416 AI277223 AW771476 F26140 AA102778 AW025780 |
| | | R44726 AA761079 AI581346 AI991909 BM005939 BE537999 BG469717 AA114156 BF437200 BE774942 |
| 453454 | 2736_1 | BC001588 BC007424 AF016369 NM_004697 BI756186 BE257019 BG500792 BI862776 AL121371 BG574833 AA703250 AA179511 |
| | | AW052006 AI280150 AI914000 AI358319 AI081204 AI082594 AA992449 AI470821 AI655744 AW237529 AA678858 AI984430 BF433055 |
| | | BE467594 BE467573 AA035630 AI289987 AI184802 AI681391 AW592416 AI138377 AI139266 AA961714 AI800163 AA418751 AW451928 |
| | | AA668676 AI273444 AI494387 BE046912 AI276555 BF196021 AA700055 AA609305 AA772596 AI635758 AI635749 H95459 AW610290 |
| | | BE464994 AA527136 BF374802 AI800175 AW195227 AI189676 BF802049 AL513632 AL554911 AL538845 BE297273 AA315321 |
| | | BM451920 BE269268 BE292835 BE018128 BG755713 BM041095 BG677009 AL039691 BF995709 BE735586 BE296453 BG393609 |
| | | BG824453 AL567522 AI745257 AW388641 AW301265 AI141144 AW029280 AU149362 AU152328 AA418960 AL121009 AI890398 |
| | | AL528748 H13050 T47086 BI000575 BF334914 BF109661 R44450 H13259 T47087 AW388646 BF305834 AL577515 BM041600 BE889299 |
| | | BF239768 |
| 442092 | 22756_2 | BM449821 BM069895 BM023170 AW025563 BG152606 BM023452 AI862106 BG959957 |
| 419175 | 35068_1 | AB018322 BC012480 BI524873 AW665554 AI934469 AI479916 BF096179 BF096162 BF096132 AA744972 AI951988 AI858339 BE076331 |
| | | AA886998 AI570585 AI916688 AI678811 AI693109 AI308135 AA669046 AA961064 AI018062 H80618 BE221942 R52609 AI915164 |
| | | AA365626 Z44671 BI052776 BF882486 BG286184 AI589558 AA931663 AA534979 AI275392 AI273455 R52553 AA829920 H80652 |
| | | AA360728 F10618 AW953666 AW176773 H85527 AA765570 AA081927 BF093262 BG743753 AL037576 AA534314 BE814964 BE973713 |
| | | N49493 BE006634 BE006630 AW270037 AA234765 AI334004 BF057179 AI857450 AI341191 AI434143 AI917449 AW517207 AA255424 |
| | | AW008334 AA847572 AA994211 AA861901 AA581873 AI580157 AI364363 AW242357 AW235291 N55645 AA319869 R36911 AA256551 |
| | | AW044188 AI203159 N49403 F02090 AI187299 AI609644 Z40516 AW952314 |
| 411605 | 10026_3 | BG256892 H10532 N46614 R52610 AW977696 BM460488 W56819 BI042183 BG977498 BE767451 BF870009 BG477472 R61137 R14274 |
| | | R20259 R09686 BI838226 BF034269 AA429173 BE741829 AW867495 AI123683 AW006831 BE831162 AW452753 AV742717 W86152 |
| | | BF115102 AI633815 BF921562 AA094230 BE092587 W86151 AA526153 AI672156 BF914496 R12579 BF852352 AA699780 T57386 |
| | | BF903022 R09933 AA678298 |
| 424036 | 6226_1 | NM_033445 BC001193 AI885781 BF794032 AA476620 AA810906 AA810905 AI291244 AI885097 AI359708 AI335629 H97396 AI344589 AA300377 |
| | | AA457566 AW771833 BE465621 AI364068 |
| | | AI364452 AI648505 AI918342 AI928670 AA886580 AL531029 AA886344 AI186419 BG329096 BM045465 AL531028 BG437151 BE868021 AA179427 |
| 432908 | 452541_1 | AF150424 AI861896 AA570057 AV738855 |
| 447197 | 2176805_1 | R36075 R36167 AI366546 |
| 443695 | 20416_12 | BE535598 AW204099 AW301249 AA609749 BF917914 AA775742 AV646137 AV646389 AA314747 |
| 430935 | 15297_3 | BC017923 AA789302 AW466994 BF513878 AI819642 AI184913 AW469044 AI220572 AW072916 AI280239 AI473611 AW841126 D60937 AA489195 N59350 AA693435 |
| | | BG531204 AA484243 AW514092 |
| 441623 | 3362_1 | BC022413 BE395396 BF754175 AA506621 BE706665 BE706678 AA723159 BE153169 BE706729 BE706558 BE153312 BE706706 |
| | | AW371853 AW371849 BE153241 BC017410 AI337912 AI090244 AW090300 BE219837 AI623661 BE501576 BE501734 AI742232 |
| | | AI023964 AI458424 AA975373 AI288904 AI984583 AA890325 N32562 AI358102 AW241694 AI038448 AI672071 AI018389 AA576391 |
| | | AA977874 AW189392 W37448 AA612894 AI277548 H89551 AI699774 H89365 AA315805 AW579186 BC014584 BC014581 AW780125 |
| | | AI672414 BE328145 AW600919 BF031306 AW172758 BE708322 AA345675 BE875779 H28241 H25318 BF540913 BG179688 BF110202 |
| | | AA528775 W37573 BE041644 AW366504 BM129522 BM129822 AI122760 BE718200 AW887496 AA149420 BE706307 BE539395 |
| | | BE748765 AI373653 R75904 BF979185 BF691393 BG495595 BI094458 BE706702 BG496559 BF248373 BG494800 |
| 407604 | 43771_1 | AK074129 AL138071 AI806793 AI453544 AI084616 BF062435 BE672932 AI828296 BF062464 AI803443 AI264326 AI280956 AI743547 |
| | | AW295227 AW139967 AI972561 AI863570 AW062752 AW062756 AW062753 AW176904 AW062755 AW062750 AW062749 AW176886 |
| | | AI751694 BG251391 AA043621 W30872 BI524827 N94581 AI084614 AA776976 AI377572 AI751695 AI016576 AW439994 AW194255 |
| | | AW627763 AA668249 AI079870 AW022524 AI968540 AI248360 AA702490 AA043262 AA631511 AW176883 AW751767 AW176887 |
| | | AW062748 AW062745 AW176862 AW176815 AW176814 AW176846 AW176841 AW176871 AW176874 AW176819 AI927437 AW191962 |
| | | R36548 AA853713 AW196059 AA618567 AW176881 AW062754 AW062751 AW062746 AW176888 AW751772 AW176859 AW751765 |
| | | AW751762 AW176833 AW176873 AW751745 AA694513 AA780815 AW176857 AW176834 AW176839 AW751771 AW176878 AW176843 |
| | | AW176842 AW751755 AW176847 AW176812 AW176830 AW176849 AW176844 AW176877 AW751757 AW176850 AW176851 AW751758 |
| | | AW751746 AW176858 AW176853 AW751766 AW176845 AW176872 AW176840 AW176876 AW176852 AW751754 AW176836 AW176856 |
| | | AW751773 AW751760 AW751751 AL138070 AA639738 AL602588 AA853712 AA317570 R38469 AW291569 |
| 458748 | 701190_1 | AI381530 BG942599 AW270510 AW265104 |
| 449444 | 2735_1 | U59185 NM_004696 AV734324 AI245349 AA369517 H88760 D79128 AA970406 H01059 H88761 H03446 BG620383 AU135008 AU136895 |
| | | AU158158 AU155762 R73608 R65751 R23756 N74630 AW078687 BE439761 BE786351 R68994 BE785867 AW297502 AW297553 BG431545 AW814843 BF382644 BG429539 |
| | | BE929862 BF811258 |
| 437834 | 294580_1 | BG110129 AW749287 BE535498 AW749299 AW749293 AW749302 AW749298 AW749291 AW749294 AW749289 AW749288 AW749296 AA769294 AW749297 |
| | | AW749295 AW749292 BE002573 |
| 430539 | 31268_1 | AK001489 AU129447 BF959274 BG565452 AI245327 AU116848 BF358559 BF358554 BF358570 BG678119 AL515852 AU154607 |
| | | AI357567 AW874359 AI122554 AA406478 AI091013 AI866679 AI686163 AA662158 AA911580 D31095 AI302576 BF588761 AU151560 |
| | | AU143828 AI291610 AW169600 D31161 AA905362 C21179 BE327258 D31474 AW439053 D31309 BF756901 BI838626 BF979839 |
| | | AU149562 BM142116 AU156455 AA452028 AW473972 AW468490 AA410271 AI475944 BF821859 AA658188 AI360390 AA226320 F37355 |
| | | F27660 F36093 AA152126 BF930021 BF375775 AW821784 AW975085 W16475 D31031 BG696392 AW860676 AW752864 BI013705 |
| | | BF965715 BF326604 AW821786 |
| 437204 | 28243_1 | AL110216 AI816283 AI688476 AA928351 AW157672 AI288740 BE468242 AI452476 AI291665 AW192364 BE503635 AW592672 AA857393 |
| | | AW162770 AI680660 AI879565 AI347717 BF880800 AI266646 AA834381 AI291667 AI264567 Z38449 T03352 AI929432 BE673697 |
| | | AI124770 AI350389 AA910378 R54042 AV746670 AW089037 AV729218 H28971 R38549 AI692560 BE671562 BE669916 BE672206 |
| | | BF445152 BF445151 AI914323 AI684406 AA932348 BE670597 AA932080 |
| 440282 | 21425_1 | NM_021188 U90919 BM458077 BG214941 AL562127 BG197712 AW994207 AA043114 AW993474 AA631832 AI911529 BI001014 |
| | | BE156322 BG319454 H27376 BG220618 BG189474 BG325006 AL519085 BM472894 AL520560 BG575806 AA319317 BG621802 |
| | | BG912722 AV760869 AV760871 R13243 BE274097 BG327062 BI765172 BE614338 BG214923 BM153391 AU147675 N58119 AL043557 |
| | | AL043558 AL043510 AA773474 AU148079 BF062869 AW189972 AW250398 AI028605 AI434688 AW149344 AI246783 AI961376 AI698209 |
| | | AA251535 AU160747 AA579811 AI572570 AI656228 AA426093 AA127171 BF222487 AA725576 F21161 AI932217 BF222251 AI024660 |
| | | AA780998 AI538862 AW140038 AI122909 AA493515 AA401920 Z39789 F10942 F05081 AA814868 F10376 AA776314 AA993104 T94991 |
| | | AW583990 AA128211 F03074 AL579261 AL519084 AA384895 BG196670 AW083526 BF220181 BE669988 BF907279 T23499 AL520559 |
| | | AU149005 N38766 H96506 AA040865 AI263737 AA723350 AI168145 AA652386 AI374699 AA487377 AI927374 AI985531 AI352105 |
| | | AI040092 AA486562 BM053020 Z41738 BE243710 AA601558 BM052743 AI445827 AA487260 AI982679 AW130546 AI743384 AI335647 |
| | | AW978172 R40324 AV736436 BE966360 AI185467 AI367193 N75025 AW131068 BE940519 H43910 AA486659 BE614365 BF220261 |
| | | BE181357 BG189473 BG210176 H43958 |
| 450251 | 40382_4 | BE080483 AI689298 BE080416 |
| 441181 | 31840_3 | AK027315 NM_032472 BG195928 BG940337 AA742244 BG195277 AA424400 AA812029 BG220715 AA416925 AA724544 BG755237 BG489761 |
| | | BG340645 BE696471 |
| 400205 | 2538_1 | NM_006265 D38551 X98294 BM477931 BM461566 AU123557 AU133303 AU134649 AW500421 BM172439 AW500587 AW503665 |
| | | AW504355 AW503640 BM152454 AW505260 AI815984 AW504075 AW500716 AL597310 BC001229 BM474371 AA984202 AU135205 |
| | | BE090841 AW163750 BF747730 BF898637 AI206506 AV660870 AV692110 AW386830 AV656831 N84710 AW993470 BF086802 |
| | | BF758454 BG960772 BF757769 BI870853 BE018627 C75436 AW148744 BF757753 BG622067 BE909924 AA708208 BG530266 BF968015 |
| | | AW992930 BF888862 BG536628 AA143164 AW748953 BG498922 BF885190 BF889005 BF754781 BF800003 BM476529 AI627668 |
| | | AW028126 AL046011 BF590668 AI017447 AA579936 AI367597 AA699622 BE280597 AI124620 AI082548 AW274985 AA677870 AI056767 |
| | | BE551689 AA287642 H94499 AI752427 AI652365 AW002374 AW062651 AA360834 N68822 AU135442 AU125960 Z78334 BE545813 |
| | | AI092115 BF312771 BF242859 BG533616 BG533761 BG164745 BG492433 BM473183 AA172043 AA172069 AU157092 AU151353 |
| | | AU155318 BE302211 AI375022 AA085641 AU157923 H88858 AA132730 AA115113 AA909781 AI475256 AA424206 AW572383 AW084296 |
| | | AI184820 AI469178 AA782432 H92184 AA340562 BF195818 AA852821 AW576342 AA827107 AA173317 AW190014 AI918514 AA729372 |
| | | AA729718 AI055958 AA331424 BE328601 AA515690 BI018896 AW628277 AA748368 AA626222 BG492636 AW380620 BF800058 |
| | | AW370956 AA290909 R25857 BG952995 BF801437 AA172077 AU155890 AU149783 AI720904 AA902936 AA865727 AI470830 AV740677 |
| | | AA142982 AA482485 AU145485 AW576399 AU156042 R63448 BF246427 BE928472 D25910 BF758439 BF968785 BE565238 AA355981 |
| | | AI905607 BG291148 BG533096 BG532888 BF030886 BG613756 BE928471 BG574501 AA187596 AA361196 T95557 BG531446 |
| | | BG527242 BG527513 BG611106 AA085995 BF847252 BG024608 BE540261 BG531236 AL579993 BG108733 BG483503 BG571032 |
| | | BG492505 |
| 447881 | 44623_1 | AK074291 AW293424 BE676135 AI832125 BE019146 BE465019 AI761124 AA617778 AI279232 AW575897 AI672039 F28618 BF924261 AA722184 BF934174 |
| | | BE004328 AV749301 BE880282 BI019798 BI019389 BF928776 AW813409 AV726604 AA077560 BE272975 BF949119 AW814195 BE879126 AI697926 BF594155 BE205787 |
| | | BF063513 N35828 AI948557 AI433839 AI379679 BG056182 AI589094 N23123 AA588805 AW316581 AI080272 AI421980 AI493318 BF194830 N87590 AA495993 N32996 |
| | | AA699844 H96845 H96592 N28741 BI035539 BF747723 BF171066 W01350 H05495 AI243785 Z39622 AA887432 AI350659 R46102 |
| 400277 | 170_1 | Y00281 NM_002950 BC010839 BC007995 BG675232 BM468552 AL555484 BG831516 BF035300 BG677277 BF852972 BE314901 |
| | | BF850656 AI371816 AA292474 AA375747 AA308414 BM454544 BI333370 BM049921 BI461428 BI465007 BI223401 BE856245 AW821164 |
| | | BF914775 BF914761 AU125835 BI222678 BI091137 BF340536 BM462798 BI224452 BG707915 AL569160 AA443815 AW572867 |
| | | AW363410 BF739268 BG010283 BI013120 BF818845 BF763468 AA305165 AI630370 AA039826 R24906 H02046 T96891 BF981330 |
| | | AW936510 AA478169 H04587 BG166574 BI869342 BE562482 BE539637 AA165089 AL579118 AL553699 BE044054 AW117440 AI520674 |
| | | BF435417 AW245648 AI952404 T29534 AU153459 AU152168 AW591591 AU146918 AI393187 AA478013 AU148143 AI224471 AI640728 |
| | | AI871537 AW264752 N93787 AI189357 AV756134 AI471659 AU147466 AA779206 AU149419 AU149104 AU159135 AA312221 AW445119 |
| | | AW021912 AI799771 F04407 AI285530 AI914643 AW068751 AA513325 AA164627 AA639285 AA569644 T96892 AI923594 BF439180 |
| | | BI770936 BF032438 AU154884 AA682793 AW072992 AU158815 AI884444 AL048031 AU158922 AU152546 AI695187 AL048033 AI245650 |
| | | AU148507 AW467451 BE536868 BF913001 BF062707 AL573082 AW067993 AA523354 BE886727 AI890705 AU159092 AI982693 |
| | | AI817553 AA236729 AI687858 BG163767 AI524675 AI678155 AA127100 AI762661 AU159718 AI469720 AA483627 AW131696 R26868 |
| | | AI199885 AW875614 AW938694 AW578974 BI763988 BG819168 BE874767 BG978292 BE162948 AL555483 AW189719 T56783 AI018819 |
| | | AI476552 BI492837 AI824440 BG996262 AA932887 AI380726 R79530 AA622108 AI262575 T56782 R27437 BE784153 AW129549 |
| | | AI675567 AI866759 BG987935 |
| 453295 | 10026_2 | BE839522 BG435352 T92108 BF110411 BE218830 BF433169 AI123554 BE465150 BE670689 BF447917 BE218141 AW204147 AI680839 |
| | | BF440050 AW770504 AW770660 AW418617 BF196503 AA053779 AI128652 AW117727 AI375178 AW071507 AI399890 AW662064 |
| | | AI401276 AW341172 AI197958 AA749065 AW168735 AW615171 AA908390 AI300043 AA233865 AI174331 AI783832 AI202132 AA723885 |
| | | N34922 N34888 AW612097 BF062429 AA034410 N71077 AA232810 AA826658 AA773393 H27681 AW071706 AA702830 R01133 H58727 |
| | | H27682 AW750643 R43716 R40566 H10351 R52554 BG120686 AW842476 |
| 439574 | 689966_1 | BG532820 AW246001 AI469788 AI350090 AI446788 BE549330 W84862 AA837988 |
| 414405 | 112689_1 | AL047596 AA393792 AI670731 AI037957 AW874364 AI038137 N62286 AI241379 BE501096 AW090696 AI927369 AI669226 AI369437 |
| | | AI371075 AW612409 AI686711 AI183289 AA477717 AI076122 AA635190 AA700984 AA781508 D81020 BF575223 AI356183 D79312 |
| | | AI375558 H61111 BG283489 BE090666 BE090664 BE090662 H26545 |
| 414051 | 1630028_1 | BE246216 BE244127 |
| 407198 | | |
| 412537 | 14066_1 | AK025201 AA425472 AI694282 BG057305 AA907787 AI286170 AI684577 AJ420494 AI809865 BF058095 AI478773 AI160445 AL044114 AW665529 AI129239 |
| | | AW297152 AI268215 AI469807 AI969353 BE552356 N66509 AA736741 AA382555 AW075811 AV759188 BI259364 BF445142 BG232065 AI141758 AI631202 AI167566 |
| | | AI208445 AA889823 BF982682 N90322 BI090882 BF208005 AW953918 AL044113 AI016793 AA382556 AW235763 AA927051 AI862075 BE886691 BE619282 |
| 442961 | 60316_2 | BE966247 BE220885 BE467384 BE350135 BE672094 AI811582 AW665254 AA772731 AI283601 AA417067 AW197746 AI868357 AI792143 AA931120 AI758506 |
| | | AA843761 BE737582 AW379586 N38812 BG567321 H13257 |
| 438869 | 52134_1 | AF075009 R63109 R63068 |
| 419502 | 5010_2 | D17251 M26878 AL579998 AL564415 AL531236 AL580013 AL531803 AL564796 BG564725 BG566337 BG617057 AL564772 BG566858 |
| | | AW950670 AV681936 BG562894 AL564428 AW452959 AL564767 AL531808 AW805054 BG562392 AV656001 AA011414 AL579726 |
| | | T72793 AL531610 AL579889 T68044 BG616669 AL531632 AI017721 AW772543 AL564742 T73787 T64764 AL564460 T68170 AL564715 |
| | | AL532514 AL531830 T67832 T69440 AL564692 T53939 T64121 AL564508 T60385 AL564753 AA342489 AA343045 AA341907 T64595 |
| | | T64309 T58898 T71857 AA342807 T64305 T71916 T72079 AA341964 T41006 T56035 T67820 T71870 T71895 T61875 T71958 R06796 |
| | | T68091 T71289 T72525 T71959 T62764 T73729 T68607 T73341 T61350 T61376 T67922 T68707 T60476 T69293 T69283 T73425 T68107 |
| | | T73317 T53747 T68301 T62888 T67862 T69420 AL564633 AA343060 T71914 T39648 T73952 T61277 T58786 T73400 T67864 T73277 |
| | | T69445 T73137 T67755 T67736 T64425 BG567196 R93196 T82897 T51176 BG567088 T64232 T71715 T69081 T62136 |
| 438714 | 2576235_1 | AA814859 AI582623 AA814857 |
| 446019 | 658727_1 | AI362520 D25917 AI670784 AI742347 AW269789 AI270700 AW610541 AW793036 AW793035 AW610540 AW362220 AW362166 |
| | | AW362214 AW362225 AW362228 AL119827 |
| 415989 | 10194_1 | BC013389 BC017398 AI023543 AA191424 AI267700 AI469633 AW958465 AW953397 AA172056 BE940298 BF909208 BF909980 |
| | | BF095153 BG285837 AI720344 BF541715 AA355086 AA172236 |
| 432339 | 57992_1 | BG823745 AW411259 BG111139 BM041741 AW015049 AW068953 AW014085 AA969360 AI027260 AI948416 AW015886 AA918278 |
| | | AA705292 AA702483 AA534205 BE856934 AI129462 BE926765 AI684275 R52686 N34869 H23555 |
| 429588 | 1396089_1 | AI092511 AI080271 AA455177 AI381859 AI381721 |
| 452279 | 11990_1 | AF392454 AK023074 AI884890 AI814455 AW966220 BF736545 AA026021 AA286843 AA251918 BG197710 AA026294 AA337356 |
| | | BC010422 AK023226 NM_022776 BM459496 AA769310 AI826460 AU153650 AU160375 AW166211 AW292992 BF433538 AI823888 |
| | | AI684798 AI655985 AW770982 AI400454 AI276257 AA639510 AI689818 AW772604 AA807639 AU130298 AU132028 BF900889 BF904822 |
| | | AY007102 AU143256 BG621460 AA829630 AI864665 AI084922 AA025234 AI360060 AA766554 AA026295 AA825817 AA251762 |
| | | BE180751 BM464530 AV715833 AA779447 AI452519 AW418525 AA435643 D25894 AA435651 AA286844 N64369 AI702262 BG288063 |
| | | BE170545 BM466232 BE299160 AA169573 AL567428 BF217285 BE967276 |
| 412560 | 72553_1 | BF002870 AI003925 AI082639 AA194383 AA702993 AI767866 AL575096 BF593252 AI948584 AI678666 BI963722 AI765219 AA620965 |
| | | BE671938 AI004689 AI004690 AI990303 AI127228 BE856290 AW203978 AI934786 AI770075 AI144132 AA812597 AI813349 AI142908 |
| | | BE671242 AI208243 H82735 BF115200 AJ345984 BE044308 F32992 AJ346047 F24958 AJ346565 AJ346456 F18071 R24502 BI830577 |
| | | BI222716 H83611 AA507760 BE463806 AA194467 AI865963 BF434962 C04894 AA813511 AA112966 N89963 |
| 433586 | 32908_1 | BC011194 AW517087 AA601054 T85512 |
| 412811 | 17212_1 | AK055716 AL520498 AL522781 BE047862 AI424991 AI863108 AA598689 AW016452 AL520499 AW957730 AA352014 BE263253 Z41952 AA121201 AV753206 |
| | | AI693507 AI091148 AA599060 AI364268 AI241940 AW089149 AW090733 AW088875 Z38240 BG958294 AA813482 AA121202 BG992558 H06383 BF435657 BE857533 |
| | | R39887 AA620528 R41807 R17734 H06382 R13591 D60420 BG958640 |
| 420439 | 35849_1 | BG614459 BE089799 R26499 AW577415 BG392326 AI034474 AA281692 BI492660 AI813489 AI762456 AI669527 BM069794 AW055057 |
| | | AW438593 AA702328 AA262137 AI379307 AA281632 AA694381 AI422791 AW243902 AA493261 AI868989 D62032 BG529976 BE504426 |
| | | AA369546 AW869612 AW021723 AW965366 AW117912 AI742020 AW970160 AA514538 AW449526 AW082476 N99706 AA262413 |
| | | AA333518 BG822620 AA643040 AW236597 BE220653 AA768827 AI081788 AA877024 N71978 AA612940 AI290151 AW082653 AI383270 |
| | | C16425 AA722211 AI539327 AA369545 AA522467 N93082 AI371310 AW082649 AW270041 AI292303 BF856785 D19612 AI701667 |
| | | R50985 AW869643 |
| 452598 | 2741_1 | AK055715 AI129800 AW027959 AI927949 AI650270 AW514661 AI708393 AL138076 AI926721 AA749139 AI399955 AW874011 AI302816 |
| | | AI242763 AA828301 AA262795 AI093249 AI084565 AA904444 BE245661 AI810709 N30447 AI653617 AI289975 AI474418 AA737455 |
| | | AI619613 AA291708 AI640264 N91446 AW029069 AA680378 AA338773 BC022321 BM450359 BE439944 AA521348 AI066761 AL138075 |
| | | BM153113 BM153112 AW182953 BM147809 AI247395 AI911116 AA977587 AA291690 AI243777 F35824 AA579199 BM479711 AW961031 |
| | | H92980 H86861 AA026904 AI052445 R92739 T10789 AV49986 AL540531 AA452953 BM152599 AA382769 BI550695 BG432646 |
| | | AV744808 |
| 447832 | 1036795_1 | AI628543 AI433357 AW772732 |
| 449343 | 14470_1 | AK055378 BE896063 AV722833 BI032095 BF952019 BG547204 AI151418 W03542 W60401 AI346936 AA862855 W60310 N72501 |
| | | BF963436 C04881 H90060 AA001203 AI863984 AI932612 AI499771 AI187944 AA501896 AA714924 H00689 AA918680 AA573996 |
| | | AA521308 AW182868 BF996707 AA908959 AI628880 AW173363 AW665845 AA130178 AI818267 AI653663 AI828924 AA746655 AI951984 |
| | | AI635625 AI093113 AI377976 AI624029 AI418242 R76291 W92652 AI207798 AV706224 AA742467 AA641806 W61229 AA130170 |
| | | AA160170 H85007 W72474 W61163 H97873 AL047509 R76567 AA812071 H81599 AA021275 H85004 H85894 BG537537 BF830518 |
| | | W76228 W46673 Z43839 R78710 C01747 H00789 BI036345 W92828 BE150445 AW380821 AW173095 H85630 H81598 H86032 R84855 |
| | | R13223 AA774992 |
| 406687 | 0_0 | M31126 |
| 418259 | 133853_1 | BM310925 AA426110 BM310629 BF434286 AW015091 BF475996 AW118867 BE675186 AI688568 AI453594 AW590589 AI652425 |
| | | AI827969 BF056946 AI802866 AI393380 AI476224 AW590639 AW136271 AI458252 AI524726 AA843768 AA782158 AI336058 AI097532 |
| | | AW451563 AA459408 AA459633 AA418444 W23607 BG940150 AI493445 AW054729 AI221929 AI868744 AA215405 AA766713 AA621546 |
| | | BF928317 BE464132 AI990909 AW271459 AI262061 AA215404 N74332 BG940151 BG952261 AA972115 W96315 AA689586 R69057 BF766886 BE769254 W05240 |
| 434804 | 118455_1 | AA659316 H64973 AA649530 |
| 452921 | 92089_1 | AW977983 AA029637 AA029697 AA806003 BF808645 W74124 AA809498 T17497 BE549804 W79644 BF446767 AI694200 AA988927 BE047252 |
| 431912 | 610_10 | BI763666 BI517886 BI759051 AI688604 AI660552 BF588523 AW004785 AW295479 BF591117 BF002672 BF064073 AA594441 AI380340 AI700219 |
| | | AI659950 AI688540 AW296326 |
| 437866 | 34267_1 | U52054 AL581000 AA156850 AW293839 BI335865 AA024963 BF149420 BE073977 AW602574 BE164012 BE163992 BE163974 |
| | | AW402161 BM194134 AW966609 W84374 BF916380 AA385173 W84366 AA383743 BF903598 AA043776 W84421 AA778446 AW444904 |
| | | BF446960 AA837481 AV755539 AW468444 AW468002 AA811830 AA581806 AI866686 AI572124 AA687333 D20160 AA812489 AU185248 |
| | | AU186004 AA156781 AI536733 BM144850 AI471883 AA040926 BF507639 AA043777 AW874142 BE832523 BE163972 BI022546 BI021204 |
| 413413 | 26025_3 | BG261240 BG231707 AI440201 BF540868 BF240794 BE779394 BG505096 AA129208 D82298 T10758 AU155599 BF857123 BF836348 |
| 451320 | 12225_2 | AK057826 AI631982 AA224195 AI701458 AA890570 AW966562 AW071907 AI671352 AI375892 T03517 AI124088 R88265 AI084316 |
| | | BF223720 AI354686 T33652 AW205836 BE931115 AI720211 T03490 BF084055 AI372637 T15415 BG054890 AA630384 F26326 AI140719 |
| | | AA443303 T33230 T33623 T33511 T33785 AW118072 W20198 AI657180 T15734 AI419606 N90552 AA224388 T15909 T03515 D55612 |
| | | W27899 |
| 437158 | 59575_1 | AL050068 AA160485 AW173544 AW296506 AW439860 AI521563 AI702529 AI393606 AW138323 AA570109 H19504 BM021968 BF063327 BF593552 |
| | | AA630766 AI597717 AI807128 AA523012 AI356250 AW451857 AA974203 AI762577 BF512552 AW007307 BE675286 AW450602 AA962057 AW516069 AI582546 |
| | | BF221924 BF222543 AI801808 AW468599 AW000736 AI866625 AW235356 BM021837 AA911956 AI680606 W86516 T03370 AW611634 H41653 AI468349 H19588 |
| | | AW090198 AW043993 R39847 |
| 412122 | 3941_2 | AK056423 AW070901 BG222419 BF928848 AL567752 AW770702 BE160226 AW248894 AI304851 AI090802 BF514704 AI659696 |
| | | AI762084 BE018718 AI088023 BF876482 AA283824 BF742200 AI349494 AI942345 AL576641 BG959608 AA853964 AA854927 AA767839 |
| | | AI955028 BG611080 BI497416 AW947186 AI703492 AW612137 AI696372 BM016022 AW852632 N57282 BI859575 AA100379 AI879394 |
| | | AI653605 BM459051 AI446574 BG031916 BI004602 BM014817 AA725075 BE063022 BG741166 BF088949 AI937125 AW936878 |
| 433023 | 3970_8 | BE999967 BF438599 AW864793 AI802899 BE815132 AW468888 AI672189 AI052004 BF112024 AA772335 AW275054 AA573845 AI144148 AI968683 AA846676 |
| | | AA927355 H80424 AW973295 R88209 F29868 BE928871 |
| 441128 | 20932_1 | BC014072 BE328850 AI356567 AI148171 AI022165 BG149661 BF000671 AA233101 AA573721 AA447991 AW016855 AI005068 AA554071 |
| | | BF478215 AA906902 AW014761 BE905651 BE512923 BM047129 AA243852 AA232991 AA127550 AA127551 AA570256 AI473237 |
| | | BF033706 N90525 AW973623 AI359627 BG674574 BE903322 |

Pkey: Unique Eos probeset identifier number
CAT number: Gene cluster number
Accession: Genbank accession numbers

TABLE 6C

| | | | |
|---|---|---|---|
| Pkey | Ref | Strand | Nt_position |

| | | | |
|---|---|---|---|
| 406399 | 9256288 | Minus | 63448-63554 |
| 403149 | 9799833 | Plus | 25034-25185 |
| 401197 | 9719705 | Plus | 176341-176452 |
| 401451 | 6634068 | Minus | 119926-121272 |
| 405770 | 2735037 | Plus | 61057-62075 |
| 401519 | 6649315 | Plus | 157315-157950 |
| 404440 | 7528051 | Plus | 80430-81581 |
| 402736 | 9212044 | Minus | 66876-67010 |
| 401866 | 8018106 | Plus | 73126-73623 |
| 404568 | 9966995 | Minus | 92893-93116 |
| 401403 | 7710966 | Plus | 146180-146294 |
| 402812 | 6010110 | Plus | 25026-25091, 25844-25920 |
| 402802 | 3287156 | Minus | 53242-53432 |
| 404996 | 6007890 | Plus | 37999-38145, 38652-38998, |
| | | | 39727-39872, 4055 |
| 400583 | 9887611 | Minus | 201148-201272 |
| 402053 | 8083229 | Plus | 62703-63179 |
| 400880 | 9931121 | Plus | 29235-29336, 36363-36580 |
| 402408 | 9796239 | Minus | 110326-110491 |
| 401747 | 9789672 | Minus | 118596-118816, 119119-119244, |
| | | | 119609-11976 |
| 403485 | 9966528 | Plus | 2888-3001, 3198-3532, 3655-4117 |
| 403817 | 8962065 | Plus | 110297-111052 |
| 402496 | 9797769 | Minus | 8615-9103 |
| 401464 | 6682291 | Minus | 170688-170834 |
| 402855 | 9662953 | Minus | 59763-59909 |
| 406081 | 9123861 | Minus | 38115-38691 |
| 405387 | 6587915 | Minus | 3769-3833, 5708-5895 |
| 401558 | 7139678 | Plus | 103510-104090 |
| 400810 | 8567959 | Minus | 174204-174331, 175062-175205 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 7A
About 381 genes upregulated in lung metastases to the brain relative to normal body tissues

| Pkey | ExAccn | UniGeneID | UniGene Title | R1 | SEQ ID NO(s): |
|---|---|---|---|---|---|
| 427383 | NM_005411 | Hs.177582 | surfactant, pulmonary-associated protein | 4.88 | 2150 5337 |
| 431433 | X65018 | Hs.253495 | surfactant, pulmonary-associated protein | 3.75 | 2553 5474 |
| 442275 | AW449467 | Hs.54795 | Homo sapiens secretoglobin, family 3A, m | 5.20 | 3409 |
| 444342 | NM_014398 | Hs.10887 | similar to lysosome-associated membrane | 2.78 | 3591 5638 |
| 450726 | AW204600 | | HUMPSPBA Human pulmonary surfactant-asso | 3.52 | 4155 |
| 426251 | M24283 | Hs.168383 | intercellular adhesion molecule 1 (CD54) | 3.24 | 2043 5296 |
| 406698 | X03068 | Hs.73931 | major histocompatibility complex, class | 2.62 | 4751 89 |
| 431164 | AA493650 | Hs.94367 | thyroid transcription factor 1 | 5.90 | 2534 |
| 428438 | NM_001955 | Hs.2271 | endothelin 1 | 2.75 | 2257 5358 |
| 412869 | AA290712 | Hs.82407 | CXC chemokine ligand 16 | 2.50 | 680 |
| 414517 | M24461 | Hs.76305 | surfactant, pulmonary-associated protein | 27.02 | 4920 839 |
| 457200 | U33749 | Hs.197764 | thyroid transcription factor 1 | 9.15 | 4582 5812 |
| 421798 | N74880 | | N-acylsphingosine amidohydrolase (acid c | 6.86 | 1564 |
| 412870 | N22788 | Hs.82407 | CXC chemokine ligand 16 | 2.67 | 681 |
| 448133 | AA723157 | Hs.73769 | folate receptor 1 (adult) | 2.62 | 3947 |
| 421502 | AF111856 | Hs.105039 | solute carrier family 34 (sodium phospha | 7.84 | 1527 5110 |
| 427834 | AA506101 | Hs.285813 | hypothetical protein FLJ11807 | 2.57 | 2199 |
| 400328 | X87344 | | transporter 2, ATP-binding cassette, sub | 2.82 | 12 4685 |
| 427700 | AA262294 | Hs.180383 | dual specificity phosphatase 6 | 2.50 | 2185 |
| 425371 | D49441 | Hs.155981 | mesothelin | 6.28 | 1957 5259 |
| 424273 | W40460 | Hs.144442 | phospholipase A2, group X | 2.63 | 1823 |
| 414945 | BE076358 | Hs.77667 | lymphocyte antigen 6 complex, locus E | 3.49 | 894 |
| 432006 | AL137382 | Hs.272320 | Homo sapiens mRNA; cDNA DKFZp434L1226 (f | 5.44 | 2626 |
| 418067 | AI127958 | Hs.83393 | cystatin E/M | 2.99 | 1156 |
| 428970 | BE276891 | Hs.194691 | retinoic acid induced 3 (RAIG1); metabo | 3.24 | 2321 |
| 435472 | AW972330 | Hs.283022 | triggering receptor expressed on myeloid | 6.14 | 2917 |
| 425211 | M18667 | Hs.1867 | progastricsin (pepsinogen C) | 19.38 | 1936 5251 |
| 414915 | NM_002462 | Hs.76391 | myxovirus (influenza) resistance 1, homo | 2.82 | 4934 892 |
| 431630 | NM_002204 | Hs.265829 | integrin, alpha 3 (antigen CD49C, alpha | 3.27 | 2578 5483 |
| 416700 | AW498958 | Hs.343475 | cathepsin D (lysosomal aspartyl protease | 2.63 | 1014 |
| 430300 | U60805 | Hs.238648 | oncostatin M receptor | 3.07 | 2465 5441 |
| 413278 | BE563085 | Hs.833 | interferon-stimulated protein, 15 kDa | 4.60 | 705 |
| 413936 | AF113676 | Hs.297681 | serine (or cysteine) proteinase inhibito | 2.67 | 4910 765 |
| 413527 | BE250788 | Hs.179882 | hypothetical protein FLJ12443 | 2.85 | 731 |
| 435575 | AF213457 | Hs.44234 | triggering receptor expressed on myeloid | 3.06 | 2929 5566 |
| 419092 | J05581 | Hs.89603 | mucin 1, transmembrane | 3.25 | 1275 5038 |
| 454453 | AW752781 | | hypothetical protein FLJ12614 similar to | 2.86 | 4485 |
| 427621 | BE621182 | Hs.179882 | hypothetical protein FLJ12443 | 3.63 | 2173 |
| 409917 | H07989 | Hs.4302 | ESTs, Weakly similar to T29299 hypotheti | 2.50 | 418 |
| 436939 | AA853680 | Hs.5345 | arginyl aminopeptidase (aminopeptidase B | 2.71 | 3022 |
| 400245 | | | Eos Control | 2.68 | |
| 432941 | W04803 | Hs.279851 | hypothetical protein FLJ10241 | 2.50 | 2713 |
| 410731 | AK001531 | Hs.66048 | hypothetical protein FLJ10669 | 2.68 | 4855 512 |
| 429380 | AF023268 | Hs.200600 | secretory carrier membrane protein 3 | 2.64 | 2363 5403 |
| 444198 | AI345232 | Hs.153503 | hypothetical protein FLJ22529 | 2.64 | 3583 |
| 429547 | AW009166 | Hs.99376 | FGENESH predicted novel secreted protein | 2.91 | 2387 |
| 450296 | AL041949 | Hs.24756 | hepatocyte growth factor-regulated tyros | 2.59 | 4126 |
| 426410 | BE298446 | Hs.305890 | BCL2-like 1 | 2.51 | 2063 |
| 433029 | NM_014322 | Hs.279926 | opsin 3 (encephalopsin) | 2.62 | 2726 5524 |
| 413719 | BE439580 | Hs.75498 | small inducible cytokine subfamily A (Cy | 2.98 | 749 |
| 440340 | AW895503 | Hs.125276 | ESTs | 2.75 | 3284 |
| 418057 | NM_012151 | Hs.83363 | coagulation factor VIII-associated (intr | 2.73 | 1153 5002 |
| 421110 | AJ250717 | Hs.1355 | cathepsin E | 3.11 | 1481 5094 |
| 425676 | AW410656 | Hs.159161 | Rho GDP dissociation inhibitor (GDI) alp | 2.57 | 1986 |
| 444006 | BE395085 | Hs.10086 | type I transmembrane protein Fn14 | 2.92 | 3568 |
| 409420 | Z15008 | Hs.54451 | laminin, gamma 2 (nicein (100 kD), kalini | 3.28 | 368 4824 |
| 440672 | AF083811 | Hs.7345 | MAD1 (mitotic arrest deficient, yeast, h | 2.89 | 3311 5612 |
| 407242 | M18728 | | gb:Human nonspecific crossreacting antig | 2.85 | 142 4766 |
| 448641 | R31845 | Hs.21666 | insulin-like 4 (placenta) | 2.80 | 3991 |
| 430044 | AA464510 | Hs.152812 | ESTs | 2.55 | 2439 |
| 408748 | J05500 | Hs.47431 | spectrin, beta, erythrocytic (includes s | 2.59 | 302 4806 |
| 447699 | AB011116 | Hs.284251 | KIAA0544 protein | 2.62 | 3892 5696 |
| 429978 | AA249027 | | ribosomal protein S6 | 2.96 | 2433 |
| 416971 | R34657 | Hs.80658 | uncoupling protein 2 (mitochondrial, pro | 2.78 | 1044 |
| 436238 | AK002163 | Hs.301724 | hypothetical protein FLJ1301 | 2.79 | 2969 5577 |
| 413585 | AI133452 | Hs.75431 | fibrinogen, gamma polypeptide | 3.01 | 737 |
| 416448 | L13210 | Hs.79339 | lectin, galactoside-binding, soluble, 3 | 2.86 | 4961 998 |
| 419152 | L12711 | Hs.89643 | transketolase (Wernicke-Korsakoff syndro | 2.68 | 1283 5040 |
| 408663 | AA766699 | Hs.298351 | alveolar soft part sarcoma chromosome re | 2.68 | 288 |
| 439453 | BE264974 | Hs.6566 | thyroid hormone receptor interactor 13 | 3.98 | 3208 |
| 432320 | AW411066 | Hs.274351 | CGI-89 protein | 2.66 | 2654 |
| 424179 | F30712 | Hs.334573 | Homo sapiens, clone IMAGE:4285740, mRNA | 2.67 | 1812 |
| 451698 | Y16187 | Hs.26880 | endothelin converting enzyme-like 1 | 3.24 | 4241 5748 |
| 400213 | | | NM_014847*:Homo sapiens KIAA0144 gene pr | 2.59 | 2 4681 |
| 450065 | AL050107 | Hs.24341 | transcriptional co-activator with PDZ-bi | 2.60 | 4102 5725 |
| 410407 | X66839 | Hs.63287 | carbonic anhydrase IX | 2.88 | 474 4846 |
| 452827 | AI571835 | Hs.55468 | ESTs | 2.57 | 4350 |
| 437915 | AI637993 | Hs.202312 | Homo sapiens clone N11 NTera2D1 teratoca | 3.18 | 3105 |
| 447349 | AI375546 | | gb:tc23d04.x1 Soares_total_fetus_Nb2HF8_{—} | 2.71 | 3848 |
| 447131 | NM_004585 | Hs.17466 | retinoic acid receptor responder (tazaro | 3.21 | 3826 5683 |
| 447250 | AI878909 | Hs.17883 | protein phosphatase 1G (formerly 2C), ma | 2.59 | 3835 |
| 439841 | AF038961 | Hs.6710 | mannose-P-dolichol utilization defect 1 | 2.72 | 3238 5599 |
| 400203 | | | Eos Control | 2.87 | |
| 429544 | BE299343 | Hs.2430 | transcription factor-like 1 | 2.93 | 2385 |
| 407244 | M10014 | | fibrinogen, gamma polypeptide | 4.25 | 143 4767 |
| 445982 | BE410233 | Hs.13501 | pescadillo (zebrafish) homolog 1, contai | 2.71 | 3715 |
| 422083 | NM_001141 | Hs.111256 | arachidonate 15-lipoxygenase, second typ | 3.55 | 1598 5134 |
| 451063 | AW163702 | Hs.25911 | HLA-B associated transcript-2 | 2.87 | 4187 |
| 421975 | AW961017 | Hs.6459 | hypothetical protein FLJ11856 | 2.74 | 1587 |
| 407756 | AA116021 | Hs.38260 | ubiquitin specific protease 18 | 3.09 | 191 |
| 410102 | AW248508 | Hs.279727 | ESTs; homologue of PEM-3 [Ciona savignyi | 3.90 | 446 |
| 431250 | BE264649 | Hs.251377 | taxol resistance associated gene 3 | 2.57 | 2544 |
| 437387 | AI198874 | Hs.28847 | AD026 protein | 2.76 | 3062 |
| 456950 | AF111170 | Hs.306165 | Homo sapiens 14q32 Jagged2 gene, complet | 2.54 | 4571 5806 |
| 412817 | AL037159 | Hs.74619 | proteasome (prosome, macropain) 26S subu | 2.58 | 674 |
| 456031 | AA335996 | | eukaryotic translation initiation factor | 2.76 | 4526 |
| 406685 | M18728 | | gb:Human nonspecific crossreacting antig | 3.21 | 4745 83 |
| 437270 | R18087 | Hs.323769 | cisplatin resistance related protein CRR | 3.28 | 3051 |
| 416976 | BE243985 | Hs.80680 | major vault protein | 2.79 | 1046 |
| 449976 | H06350 | Hs.135056 | Human DNA sequence from clone RP5-850E9 | 3.21 | 4095 |
| 419705 | AW368634 | Hs.154331 | ESTs | 2.92 | 1351 |
| 420234 | AW404908 | Hs.96038 | Ric (Drosophila)-like, expressed in many | 3.06 | 1399 |
| 429619 | AL120751 | Hs.211568 | eukaryotic translation initiation factor | 2.60 | 2398 |
| 410575 | BE207480 | Hs.6994 | Homo sapiens cDNA: FLJ22044 fis, clone H | 2.84 | 494 |
| 458176 | AI961519 | Hs.140309 | Homo sapiens, clone IMAGE:3677194, mRNA, | 2.79 | 4613 |
| 411773 | NM_006799 | Hs.72026 | protease, serine, 21 (testisin) | 3.28 | 4867 576 |
| 437852 | BE001836 | Hs.256897 | putative GPCR | 2.60 | 3099 |
| 425236 | AW067800 | Hs.155223 | stanniocalcin 2 | 2.78 | 1941 |
| 416018 | AW138239 | Hs.78977 | proprotein convertase subtilisin/kexin t | 2.84 | 966 |
| 452732 | BE300078 | Hs.80449 | Homo sapiens, clone IMAGE:3535294, mRNA, | 2.73 | 4339 |
| 427584 | BE410293 | Hs.179718 | v-myb avian myeloblastosis viral oncogen | 2.65 | 2168 |
| 417881 | AI879117 | Hs.7991 | gb:au54g09.y1 Schneider fetal brain 0000 | 2.60 | 1135 |
| 453012 | T95804 | Hs.31334 | putative mitochondrial outer membrane pr | 2.57 | 4366 |
| 443179 | AI928402 | Hs.6933 | hypothetical protein FLJ12684 | 2.83 | 3503 |
| 432226 | AW182766 | Hs.273558 | phosphate cytidylyltransferase 1, cholin | 2.86 | 2645 |
| 458748 | AI381530 | | gb:te76d07.x1 Soares_NFL_T_GBC_S1 Homo s | 2.95 | 4635 |
| 418678 | NM_001327 | Hs.167379 | cancer/testis antigen (NY-ESO-1) | 2.67 | 1226 5024 |
| 427719 | AI393122 | Hs.134726 | ESTs | 2.54 | 2189 |
| 418399 | AF131781 | Hs.84753 | hypothetical protein FLJ12442 | 2.94 | 1196 5014 |
| 446872 | X97058 | Hs.16362 | pyrimidinergic receptor P2Y, G-protein c | 4.04 | 3796 5678 |
| 459255 | AI493244 | Hs.239500 | hypothetical protein MGC13114 | 2.79 | 4663 |
| 415989 | AI267700 | | ESTs | 2.85 | 963 |
| 435151 | AA348482 | Hs.4788 | nicastrin | 2.74 | 2901 |
| 421846 | AA017707 | Hs.1432 | protein kinase C substrate 80K-H | 2.58 | 1570 |
| 422997 | BE018212 | Hs.122908 | DNA replication factor | 2.81 | 1708 |
| 422675 | BE018517 | Hs.119140 | eukaryotic translation initiation factor | 2.53 | 1671 |
| 447733 | AF157482 | Hs.19400 | MAD2 (mitotic arrest deficient, yeast, h | 3.06 | 3897 5698 |
| 401131 | | | NM_00165*:Homo sapiens aquaporin 5 (AQP | 2.53 | 25 4696 |
| 430454 | AW469011 | Hs.105635 | ESTs | 2.93 | 2487 |
| 426318 | AA375125 | Hs.147112 | Homo sapiens cDNA: FLJ22322 fis, clone H | 2.71 | 2051 |
| 421541 | NM_003942 | Hs.105584 | ribosomal protein S6 kinase, 90 kD, polyp | 2.60 | 1536 5116 |
| 421506 | BE302796 | Hs.105097 | thymidine kinase 1, soluble | 2.64 | 1528 |
| 422532 | AL008726 | Hs.118126 | protective protein for beta-galactosidas | 2.66 | 1653 5153 |
| 451481 | AA300228 | Hs.295866 | hypothetical protein DKFZp434N1923 | 3.17 | 4225 |
| 412708 | R26830 | Hs.106137 | ESTs, Weakly similar to CGHU7L collagen | 3.23 | 661 |
| 441128 | AA570256 | | ESTs, Weakly similar to T23273 hypotheti | 3.08 | 3334 |
| 416902 | AA375634 | Hs.288974 | hypothetical protein FLJ12528 | 2.56 | 1035 |
| 402496 | | | Target Exon | 2.60 | |
| 452817 | AA322859 | Hs.284275 | Homo sapiens PAK2 mRNA, complete cds | 2.54 | 4347 |
| 437741 | BE561610 | Hs.5809 | putative transmembrane protein; homolog | 2.81 | 3085 |
| 413753 | U17760 | Hs.75517 | laminin, beta 3 (nicein (125 kD), kalinin | 3.08 | 4906 750 |
| 456977 | AK000252 | Hs.169758 | hypothetical protein FLJ20245 | 3.22 | 4572 5807 |
| 419214 | AI685324 | Hs.89709 | glutamate-cysteine ligase, modifier subu | 2.73 | 1289 |
| 446538 | AV658957 | Hs.135211 | ESTs | 2.52 | 3759 |
| 454034 | NM_000691 | Hs.575 | aldehyde dehydrogenase 3 family, member | 3.76 | 4462 5792 |
| 419663 | AA394208 | Hs.92198 | calcium-regulated heat-stable protein (2 | 3.03 | 1345 |
| 413582 | AW295647 | Hs.71331 | hypothetical protein MGC5350 | 3.14 | 736 |
| 424676 | Y08565 | Hs.151678 | UDP-N-acetyl-alpha-D-galactosamine:polyp | 2.63 | 1870 5224 |
| 427963 | AI042582 | Hs.181271 | CGI-120 protein | 2.52 | 2207 |
| 428643 | AA431383 | Hs.98701 | ESTs, Weakly similar to OSHU7B cytochrom | 4.14 | 2278 |
| 418329 | AW247430 | Hs.84152 | cystathionine-beta-synthase | 2.52 | 1186 |
| 434262 | AF121858 | Hs.12169 | sorting nexin 8 | 2.50 | 2824 5544 |
| 446766 | AF083208 | Hs.16178 | apoptosis antagonizing transcription fac | 2.96 | 3781 5676 |
| 437879 | BE262082 | Hs.5894 | hypothetical protein FLJ10305 | 3.10 | 3102 |
| 444371 | BE540274 | Hs.239 | forkhead box M1 | 3.68 | 3592 |
| 401451 | | | NM_004496*:Homo sapiens hepatocyte nucle | 2.64 | 27 4697 |
| 433485 | AI493076 | Hs.306098 | aldo-keto reductase family 1, member C2 | 3.79 | 2766 |
| 435750 | AB029012 | Hs.4990 | KIAA1089 protein | 2.89 | 2939 5570 |
| 408901 | AK001330 | Hs.48855 | hypothetical protein FLJ10468 | 2.54 | 316 4808 |
| 418526 | BE019020 | Hs.85838 | solute carrier family 16 (monocarboxylic | 3.06 | 1211 |
| 448633 | AA311426 | Hs.21635 | tubulin, gamma 1 | 2.81 | 3990 |
| 443715 | AI583187 | Hs.9700 | cyclin E1 | 3.24 | 3544 |
| 432441 | AW292425 | Hs.163484 | intron of hepatocyte nuclear factor-3 al | 3.45 | 2668 |
| 409041 | AB033025 | Hs.50081 | Hypothetical protein, XP_051860 (KIAA119 | 2.96 | 327 4811 |
| 420956 | AA351584 | Hs.100543 | Homo sapiens clone 24505 mRNA sequence | 2.73 | 1468 |
| 428844 | AW972635 | Hs.301904 | hypothetical protein FLJ12671 | 2.90 | 2311 |
| 404440 | | | NM_021048:Homo sapiens melanoma antigen, | 2.66 | 4721 54 |
| 420186 | NM_015925 | Hs.95697 | liver-specific bHLH-Zip transcription fa | 2.98 | 1392 5066 |
| 441031 | AI110684 | Hs.7645 | fibrinogen, B beta polypeptide | 3.05 | 3329 |
| 421307 | BE539976 | Hs.103305 | Homo sapiens mRNA; cDNA DKFZp434B0425 (f | 4.83 | 1506 |
| 422605 | H16646 | Hs.118666 | hypothetical protein PP591 | 2.78 | 1661 |
| 409893 | AW247090 | Hs.57101 | minichromosome maintenance deficient (S. | 2.64 | 415 |
| 440006 | AK000517 | Hs.6844 | NALP2 protein; PYRIN-Containing APAF1-li | 3.69 | 3252 5601 |
| 453902 | BE502341 | Hs.3402 | ESTs | 2.60 | 4449 |
| 424867 | AI024860 | Hs.153591 | Not56 (D. melanogaster)-like protein | 3.62 | 1891 |
| 450184 | W31096 | Hs.237617 | Homo sapiens, clone IMAGE:3447394, mRNA, | 2.50 | 4109 |
| 422756 | AA441787 | Hs.119689 | glycoprotein hormones, alpha polypeptide | 3.42 | 1679 |
| 408196 | AL034548 | Hs.43627 | SRY (sex determining region Y)-box 22 | 2.65 | 243 4790 |
| 428028 | U52112 | Hs.182018 | interleukin-1 receptor-associated kinase | 2.73 | 2215 |
| 404996 | | | Target Exon | 2.54 | |
| 414675 | R79015 | Hs.296281 | interleukin enhancer binding factor 1 | 2.78 | 861 |
| 429170 | NM_001394 | Hs.2359 | dual specificity phosphatase 4 | 3.10 | 2336 5392 |
| 440590 | AI863446 | Hs.266308 | mosaic serine protease | 2.50 | 3301 |
| 430316 | NM_000875 | Hs.239176 | insulin-like growth factor 1 receptor | 2.59 | 2470 5445 |
| 430056 | X97548 | Hs.228059 | KRAB-associated protein 1 | 2.59 | 2440 5434 |
| 448262 | AW880830 | Hs.186273 | Homo sapiens quiescin Q6 (QSCN6) | 3.89 | 3953 |
| 414907 | X90725 | Hs.77597 | polo (Drosophia)-like kinase | 2.66 | 4933 891 |
| 408116 | AA251393 | Hs.289052 | Homo sapiens, Similar to RIKEN cDNA 5430 | 2.56 | 237 |
| 426283 | NM_003937 | Hs.169139 | kynureninase (L-kynurenine hydrolase) | 4.59 | 2048 5297 |
| 454066 | X00356 | Hs.37058 | calcitonin/calcitonin-related polypeptid | 3.09 | 4467 5794 |
| 430508 | AI015435 | Hs.104637 | ESTs | 3.32 | 2490 |
| 450663 | H43540 | Hs.25292 | ribonuclease HI, large subunit | 2.97 | 4145 |
| 442577 | AA292998 | Hs.163900 | ESTs | 2.73 | 3447 |
| 453350 | AI917771 | Hs.61790 | hypothetical protein FLJ23338 | 2.65 | 4398 |
| 410444 | W73484 | Hs.132554 | gb:zd54e04.s1 Soares_fetal_heart_NbHH19W | 2.63 | 480 |
| 454057 | AW009478 | Hs.36574 | hypothetical protein FLJ21125 | 3.07 | 4465 |
| 430677 | Z26317 | | desmoglein 2 | 2.62 | 2504 5461 |
| 440457 | BE387593 | Hs.21321 | Homo sapiens clone FLB9213 PRO2474 mRNA, | 2.59 | 3289 |
| 425811 | AL039104 | Hs.159557 | karyopherin alpha 2 (RAG cohort 1, impor | 2.69 | 1999 |
| 410151 | X15723 | Hs.59242 | paired basic amino acid cleaving enzyme | 2.70 | 449 4838 |
| 420281 | AI623693 | Hs.323494 | Predicted cafion efflux pump | 2.56 | 1405 |
| 422880 | AF228704 | Hs.193974 | glutathione reductase | 2.80 | 1689 5161 |
| 424494 | U78575 | Hs.149255 | phosphatidylinositol-4-phosphate 5-kinas | 2.62 | 1852 5214 |
| 457819 | AA057484 | Hs.35406 | FLJ20522 Hypothetical protein FLJ20522 | 2.77 | 4601 |
| 440087 | W28969 | Hs.7718 | hypothetical protein FLJ22678 | 2.64 | 3260 |
| 424800 | AL035588 | Hs.153203 | MyoD family inhibitor | 3.02 | 1888 5232 |
| 412939 | AW411491 | Hs.75069 | eukaryotic translation elongation factor | 3.23 | 684 |
| 442660 | AW138174 | Hs.130651 | ESTs | 2.52 | 3458 |
| 440994 | AI160011 | Hs.272068 | ESTs | 3.34 | 3323 |
| 432026 | AA524545 | Hs.224830 | ESTs | 2.70 | 2627 |
| 429671 | BE379335 | Hs.211594 | proteasome (prosome, macropain) 26S subu | 2.75 | 2405 |
| 428758 | AA433988 | Hs.98502 | CA125 antigen; mucin 16 | 4.09 | 2292 |
| 429263 | AA019004 | Hs.198396 | ATP-binding cassette, sub-family A (ABC1 | 4.29 | 2345 |
| 426991 | AK001536 | Hs.214410 | Homo sapiens cDNA FLJ10674 fis, clone NT | 2.70 | 2117 |
| 450998 | BE387614 | Hs.25797 | splicing factor 3b, subunit 4, 49 kD | 3.02 | 4181 |
| 449207 | AL044222 | Hs.23255 | nucleoporin 155 kD | 4.28 | 4045 |
| 419897 | X90826 | Hs.93649 | upstream transcription factor 2, c-fos i | 2.59 | 1366 5059 |
| 457211 | AW972565 | Hs.32399 | ESTs, Weakly similar to S51797 vasodilat | 3.52 | 4583 |
| 419667 | AU077005 | Hs.92208 | a disintegrin and metalloproteinase doma | 3.43 | 1346 |
| 431083 | AF110400 | Hs.249200 | fibroblast growth factor 19 | 2.93 | 2530 5469 |
| 427239 | BE270447 | | ubiquitin carrier protein | 3.56 | 2134 |
| 418164 | AI761820 | Hs.41074 | ESTs, Weakly similar to 139294 McLeod sy | 4.48 | 1165 |
| 427528 | AU077143 | Hs.179565 | minichromosome maintenance deficient (S. | 2.84 | 2161 |
| 430375 | AW371048 | Hs.93758 | H4 histone family, member H | 3.26 | 2477 |
| 457465 | AW301344 | Hs.122908 | DNA replication factor | 3.03 | 4592 |
| 416000 | R82342 | Hs.79856 | ESTs, Weakly similar to S65657 alpha-1C- | 3.21 | 965 |
| 420005 | AW271106 | Hs.133294 | ESTs | 3.53 | 1372 |
| 425003 | AF119046 | Hs.154149 | apurinic/apyrimidinic endonuclease(APEX | 3.13 | 1909 5242 |
| 447033 | AI357412 | Hs.157601 | Predicted gene: Eos cloned; secreted w/V | 3.73 | 3814 |
| 453439 | AI572438 | Hs.32976 | guanine nucleotide binding protein 4 | 3.12 | 4406 |
| 428865 | BE544095 | Hs.164960 | BarH-like homeobox 1 | 3.74 | 2314 |
| 417542 | J04129 | Hs.82269 | progestagen-associated endometrial prote | 3.56 | 1101 4990 |
| 414416 | AW409985 | Hs.76084 | hypothetical protein MGC2721 | 2.72 | 824 |
| 435025 | T08990 | Hs.4742 | anchor attachment protein 1 (Gaa1p, yeas | 2.55 | 2889 |
| 419224 | NM_012189 | Hs.314452 | fibrousheathin II | 3.56 | 1290 5041 |
| 411975 | AI916058 | Hs.144583 | 3′UTR of: dead ringer (Drosophila)-like | 2.81 | 596 |
| 412856 | BE386745 | Hs.74631 | basigin (OK blood group) | 2.70 | 678 |
| 436887 | AW953157 | Hs.193235 | hypothetical protein DKFZp547D155 | 2.65 | 3018 |
| 447289 | AW247017 | Hs.36978 | melanoma antigen, family A, 3 | 3.12 | 3839 |
| 410512 | AA085603 | Hs.250570 | hypothetical protein MGC3180 | 2.69 | 487 |
| 453120 | AA292891 | Hs.31773 | pregnancy-induced growth inhibitor | 3.76 | 4375 |
| 440548 | AL117408 | Hs.7274 | DKFZP434P1750 protein | 3.15 | 3299 5609 |
| 444670 | H58373 | Hs.332938 | hypothetical protein MGC5370 | 2.68 | 3618 |
| 416478 | U38945 | Hs.1174 | cyclin-dependent kinase inhibitor 2A (me | 4.85 | 1204 5017 |
| 421526 | AL080121 | Hs.105460 | DKFZP564O0823 protein | 2.72 | 1534 5114 |
| 414774 | X02419 | Hs.77274 | plasminogen activator, urokinase | 3.51 | 4928 877 |
| 447400 | AK000322 | Hs.18457 | hypothetical protein FLJ20315 | 2.69 | 3857 5686 |
| 423063 | BE159877 | Hs.120824 | hypothetical protein FLJ21845 | 2.61 | 1714 |
| 432201 | AI538613 | Hs.298241 | Transmembrane protease, serine 3 | 3.48 | 2643 |
| 422976 | AU076657 | Hs.1600 | chaperonin containing TCP1, subunit 5 (e | 2.78 | 1705 |
| 435408 | H07897 | Hs.4302 | ESTs, Weakly similar to T29299 hypotheti | 3.29 | 2912 |
| 418506 | AA084248 | Hs.85339 | Unknown protein for MGC:29643 (formerly | 2.61 | 1208 |
| 422956 | BE545072 | Hs.122579 | ECT2 protein (Epithelial cell transformi | 3.61 | 1698 |
| 429228 | AI553633 | | hypothetical protein MGC33630 | 4.46 | 2340 |
| 415214 | AI445236 | Hs.125124 | EphB2 | 2.91 | 917 |
| 449281 | AI808699 | Hs.162717 | hypothetical protein MGC15668 | 2.79 | 4053 |
| 421305 | BE397354 | Hs.324830 | diptheria toxin resistance protein requi | 2.52 | 1505 |
| 437897 | AA770561 | Hs.146170 | hypothetical protein FLJ22969 | 3.10 | 3104 |
| 425159 | NM_004341 | Hs.154868 | carbamoyl-phosphate synthetase 2, aspart | 3.15 | 1931 5249 |
| 445654 | X91247 | Hs.13046 | thioredoxin reductase 1 | 3.98 | 3691 5658 |
| 408298 | AI745325 | Hs.271923 | Homo sapiens cDNA: FLJ22785 fis, clone K | 2.90 | 253 |
| 441362 | BE614410 | Hs.23044 | RAD51 (S. cerevisiae) homolog (E coli Re | 3.22 | 3347 |
| 420029 | BE258876 | Hs.94446 | polyamine-modulated factor 1 | 2.68 | 1375 |
| 408908 | BE296227 | Hs.250822 | serine/threonine kinase 15 | 2.65 | 317 |
| 442108 | AW452649 | Hs.166314 | ESTs | 3.36 | 3398 |
| 424905 | NM_002497 | Hs.153704 | NIMA (never in mitosis gene a)-related k | 2.69 | 1898 5237 |
| 438552 | AJ245820 | Hs.6314 | type I transmembrane receptor (seizure-r | 2.83 | 3148 5596 |
| 450074 | AI367213 | Hs.14070 | hypothetical protein FLJ14166 | 4.18 | 4103 |
| 416322 | BE019494 | Hs.79217 | pyrroline-5-carboxylate reductase 1 | 2.58 | 986 |
| 405770 | | | NM_002362:Homo sapiens melanoma antigen, | 2.71 | 4740 74 |
| 424001 | W67883 | Hs.137476 | paternally expressed 10 | 2.58 | 1788 |
| 439963 | AW247529 | Hs.6793 | platelet-activating factor acetylhydrola | 3.01 | 3250 |
| 424441 | X14850 | Hs.147097 | H2A histone family, member X | 2.63 | 1846 5212 |
| 410076 | T05387 | Hs.7991 | ESTs | 4.85 | 440 |
| 413142 | M81740 | Hs.75212 | ornithine decarboxylase 1 | 2.58 | 4894 695 |
| 448610 | NM_006157 | Hs.21602 | nel (chicken)-like 1 | 3.52 | 3989 5712 |
| 447960 | AW954377 | Hs.26412 | ring finger protein 26 | 3.56 | 3927 |
| 432415 | T16971 | Hs.289014 | ESTs, Weakly similar to A43932 mucin 2 p | 2.62 | 2665 |
| 453905 | NM_002314 | Hs.36566 | LIM domain kinase 1 | 3.30 | 4450 5788 |
| 437623 | D63880 | Hs.5719 | chromosome condensation-related SMC-asso | 2.58 | 3076 5587 |
| 453884 | AA355925 | Hs.36232 | KIAA0186 gene product | 2.87 | 4444 |
| 427715 | BE245274 | Hs.180428 | KIAA1181 protein | 3.67 | 2188 |
| 441553 | AA281219 | Hs.121296 | ESTs | 4.46 | 3357 |
| 431070 | AW408164 | Hs.249184 | transcription factor 19 (SC1) | 2.87 | 2529 |
| 421254 | AK001724 | Hs.102950 | coat protein gamma-cop | 2.59 | 1496 |
| 451807 | W52854 | | hypothetical protein FLJ23293 similar to | 2.99 | 4249 |
| 411248 | AA551538 | Hs.69321 | Homo sapiens cDNA FLJ14408 fis, clone HE | 3.42 | 541 |
| 418054 | NM_002318 | Hs.83354 | Iysyl oxidase-like 2 | 2.50 | 1152 5001 |
| 452259 | AA317439 | Hs.28707 | signal sequence receptor, gamma (translo | 2.56 | 4290 |
| 423073 | BE252922 | Hs.123119 | MAD (mothers against decapentaplegic, Dr | 2.56 | 1715 |
| 407792 | AI077715 | Hs.39384 | putative secreted ligand homologous to f | 2.60 | 196 |
| 432078 | BE314877 | Hs.24553 | hypothetical protein FLJ12541 similar to | 3.57 | 2629 |
| 410553 | AW016824 | Hs.272068 | hypothetical protein MGC14128 | 4.37 | 491 |
| 418113 | AI272141 | Hs.83484 | SRY (sex determining region Y)-box 4 | 3.60 | 1161 |
| 413063 | AL035737 | Hs.75184 | chitinase 3-like 1 (cartilage glycoprote | 2.68 | 692 |
| 446557 | U68566 | Hs.15318 | HS1 binding protein | 2.60 | 3761 5669 |
| 416294 | D86980 | Hs.79170 | KIAA0227 protein | 2.71 | 4958 984 |
| 452613 | AA461599 | Hs.23459 | ESTs | 2.67 | 4329 |
| 431836 | AF178532 | Hs.271411 | beta-site APP-cleaving enzyme 2 | 2.53 | 2603 5491 |
| 417720 | AA205625 | Hs.208067 | ESTs | 2.58 | 1116 |
| 438915 | AA280174 | Hs.285681 | Williams-Beuren syndrome chromosome regi | 3.95 | 3178 |
| 429610 | AB024937 | Hs.211092 | LUNX protein; PLUNC (palate lung and nas | 7.69 | 2395 5415 |
| 409015 | BE389387 | Hs.49767 | NM_004553:Homo sapiens NADH dehydrogenas | 3.05 | 323 |
| 415279 | F04237 | Hs.1447 | glial fibrillary acidic protein | 4.05 | 923 |
| 448296 | BE622756 | Hs.10949 | Homo sapiens cDNA FLJ14162 fis, clove NT | 2.54 | 3956 |
| 418960 | NM_004494 | Hs.89525 | hepatoma-derived growth factor (HDGF) | 2.54 | 1263 5034 |
| 446204 | AI279809 | Hs.150019 | ESTs | 2.81 | 3735 |
| 413004 | T35901 | Hs.75117 | interleukin enhancer binding factor 2, 4 | 3.03 | 688 |
| 425944 | AK000654 | Hs.164256 | hypothetical protein FLJ20657 | 2.62 | 2013 5283 |
| 425057 | AA826434 | Hs.1619 | achaete-scute complex (Drosophila) homol | 4.87 | 1915 |
| 452461 | N78223 | Hs.108106 | transcription factor | 2.61 | 4311 |
| 453210 | AL133161 | Hs.32360 | hypothetical protein FLJ10867 | 2.69 | 4387 5772 |
| 411825 | AK000334 | | solute carrier family 39 (zinc transport | 4.19 | 4868 580 |
| 425234 | AW152225 | Hs.165909 | ESTs, Weakly similar to I38022 hypotheti | 3.15 | 1940 |
| 437016 | AU076916 | Hs.5398 | guanine monphosphate synthetase | 3.68 | 3027 |
| 407137 | T97307 | | gb:ye53h05.s1 Soares fetal liver spleen | 2.69 | 128 |
| 409757 | NM_001898 | Hs.123114 | cystatin SN | 3.47 | 403 4832 |
| 444706 | AK000398 | Hs.11747 | hypothetical protein FLJ20391 | 2.80 | 3621 5643 |
| 430393 | BE185030 | Hs.241305 | estrogen-responsive B box protein | 2.53 | 2480 |
| 452838 | U65011 | Hs.30743 | preferentially expressed antigen in mela | 6.17 | 4353 5765 |
| 447532 | AK000614 | Hs.18791 | hypothetical protein FLJ20607 | 2.66 | 3875 5690 |
| 424140 | Z48051 | Hs.141308 | myelin oligodendrocyte glycoprotein | 3.54 | 1809 5200 |
| 436217 | T53925 | Hs.107 | fibrinogen-like 1 | 4.92 | 2968 |
| 452833 | BE559681 | Hs.30736 | KIAA0124 protein | 3.80 | 4351 |
| 422765 | AW409701 | Hs.1578 | baculoviral IAP repeat-containing 5 (sur | 2.90 | 1680 |
| 443426 | AF098158 | Hs.9329 | chromosome 20 open reading frame 1 | 3.20 | 3523 5627 |
| 409269 | AA576953 | Hs.22972 | steroid 5 alpha-reductase 2-like; H5AR g | 4.09 | 358 |
| 422656 | AI870435 | Hs.1569 | LIM homeobox protein 2 | 3.01 | 1668 |
| 423551 | AA327598 | Hs.89633 | ESTs | 2.60 | 1757 |
| 409187 | AF154830 | Hs.50966 | carbamoyl-phosphate synthetase 1, mitoch | 4.21 | 347 4816 |
| 409557 | BE182896 | Hs.3686 | ESTs | 2.53 | 384 |
| 416984 | H38765 | Hs.80706 | diaphorase (NADH/NADPH) (cytochrome b-5 | 3.58 | 1047 |
| 442173 | N76101 | Hs.8127 | KIAA0144 gene product | 2.77 | 3402 |
| 408452 | AA054683 | Hs.192455 | ESTs, Weakly similar to ALU7_HUMAN ALU S | 4.18 | 268 |
| 428342 | AI739168 | | Homo sapiens cDNA FLJ13458 fis, clone PL | 2.79 | 2244 |
| 426691 | NM_006201 | Hs.171834 | PCTAIRE protein kinase 1 | 3.68 | 2088 5314 |
| 438956 | W00847 | Hs.135056 | Human DNA sequence from clone RP5-850E9 | 3.56 | 3180 |
| 456508 | AA502764 | Hs.123469 | ESTs, Weakly similar to AF208855 1 BM-01 | 2.93 | 4547 |
| 449027 | AJ271216 | Hs.22880 | dipeptidylpeptidase III | 2.95 | 4030 5719 |
| 431846 | BE019924 | Hs.271580 | uroplakin 1B | 3.78 | 2605 |
| 419741 | NM_007019 | Hs.93002 | ubiquitin carrier protein E2-C | 3.63 | 1355 5056 |
| 416361 | AW204907 | Hs.6872 | ESTs, Weakly similar to CA13_HUMAN COLLA | 2.94 | 989 |
| 407355 | AA846203 | Hs.193974 | ESTs, Weakly similar to ALU1_HUMAN ALU S | 2.60 | 155 |
| 424954 | NM_000546 | Hs.1846 | tumor protein p53 (Li-Fraumeni syndrome) | 2.77 | 1901 5238 |
| 407754 | AA527348 | Hs.288967 | Homo sapiens cDNA FLJ14105 fis, clone MA | 3.20 | 190 |
| 419875 | AA853410 | Hs.93557 | proenkephalin | 3.46 | 1365 |
| 413437 | BE313164 | Hs.75361 | gene from NF2/meningioma region of 22q12 | 2.54 | 722 |
| 417389 | BE260964 | Hs.82045 | midkine (neurite growth-promoting factor | 3.09 | 1091 |
| 444381 | BE387335 | Hs.283713 | hypothetical protein BC014245 | 2.80 | 3593 |
| 453968 | AA847843 | Hs.62711 | High mobility group (nonhistone chromoso | 5.29 | 4456 |
| 427954 | J03060 | Hs.247551 | metaxin 1 | 3.30 | 2206 |
| 438746 | AI885815 | Hs.184727 | Human melanoma-associated antigen p97 (m | 2.57 | 3164 |
| 422168 | AA586894 | Hs.112408 | S100 calcium-binding protein A7 (psorias | 2.73 | 1612 |
| 425842 | AI587490 | Hs.159623 | NK-2 (Drosophila) homolog B | 3.17 | 2002 |
| 448993 | AI471630 | | KIAA0144 gene product | 2.70 | 4023 |
| 428004 | AA449563 | Hs.151393 | glutamate-cysteine ligase, catalytic sub | 2.69 | 2213 |
| 425848 | BE242709 | Hs.159637 | valyl-tRNA synthetase 2 | 3.79 | 2004 |
| 424458 | M29273 | Hs.1780 | myelin associated glycoprotein | 2.69 | 1848 5213 |
| 419078 | M93119 | Hs.89584 | insulinoma-associated 1 | 5.07 | 1272 5036 |
| 430294 | AI538226 | Hs.32976 | guanine nucleotide binding protein 4 | 2.82 | 2463 |
| 429922 | Z97630 | Hs.226117 | H1 histone family, member 0 | 2.52 | 2427 5430 |
| 409103 | AF251237 | Hs.112208 | XAGE-1 protein | 7.18 | 333 4812 |
| 424629 | M90656 | Hs.151393 | glutamate-cysteine ligase, catalytic sub | 3.19 | 1865 5221 |
| 414313 | NM_004371 | Hs.75887 | coatomer protein complex, subunit alpha | 2.55 | 4916 809 |
| 416138 | C18946 | Hs.79026 | myeloid leukemia factor 2 | 2.50 | 976 |
| 434067 | H18913 | Hs.124023 | Homo sapiens cDNA FLJ14218 fis, clone NT | 5.38 | 2812 |
| 411908 | L27943 | Hs.72924 | cytidine deaminase | 3.32 | 4869 585 |
| 428484 | AF104032 | Hs.184601 | solute carrier family 7 (cationic amino | 2.57 | 2265 5364 |
| 422095 | AI868872 | Hs.282804 | hypothetical protein FLJ22704 | 2.83 | 1603 |
| 430791 | AA486293 | Hs.272068 | ESTs, Weakly similar to ALU3_HUMAN ALU S | 3.14 | 2513 |
| 428839 | AI767756 | Hs.82302 | Homo sapiens cDNA FLJ14814 fis, clone NT | 2.51 | 2310 |
| 424481 | R19453 | Hs.1787 | proteolipid protein 1 (Pelizaeus-Merzbac | 2.66 | 1851 |
| 447342 | AI199268 | Hs.19322 | Homo sapiens, Similar to RIKEN cDNA 2010 | 2.74 | 3845 |
| 417900 | BE250127 | Hs.82906 | CDC20 (cell division cycle 20, S. cerevi | 3.04 | 1136 |
| 411305 | BE241596 | Hs.69547 | myelin basic protein | 4.08 | 546 |
| 406690 | M29540 | Hs.220529 | carcinoembryonic antigen-related cell ad | 3.07 | 4748 86 |
| 418738 | AW388633 | Hs.6682 | solute carrier family 7, (cationic amino | 4.00 | 1234 |
| 427344 | NM_000869 | Hs.2142 | 5-hydroxytryptamine (serotonin) receptor | 4.24 | 2145 5336 |
| 433447 | U29195 | Hs.3281 | neuronal pentraxin II | 2.98 | 2764 5536 |
| 419395 | BE268326 | Hs.90280 | 5-aminoimidazole-4-carboxamide ribonucle | 3.04 | 1310 |
| 424420 | BE614743 | Hs.146688 | prostaglandin E synthase | 3.54 | 1842 |

Pkey: Unique Eos probeset identifier number
ExAccn: Exemplar Accession number, Genbank accession number
UniGeneID: UniGene number
UniGene Title: UniGene gene title
R1: 90th percentile of lung metastases to the brain Als divided by the 90th percentile of normal body tissues Als, where the 15th percentile of all normal body tissue Als was subtracted from the numerator and denominator.
SEQ ID NO(s): SEQ ID number(s) for nucleic acid and protein sequences associated with table entry.

TABLE 7B

| Pkey | CAT number | Accession |
|---|---|---|
| 450726 | 666520_1 | AI732297 AW204600 T95017 |
| 421798 | 3042_4 | BC017829 AW276646 AI984209 AA663933 AA634104 AA551528 AA634041 |
| | | AA298038 BG483990 T89297 BF853958 H64685 T90329 T60644 T57747 BF852694 T92529 |
| | | BG482852 BF883064 BF883066 N74880 AA829796 N90716 |
| 454453 | 8582_4 | BF313069 BE879305 AW752781 AW752727 AW752559 AW752578 AW752584 |
| | | BF846118 AL545903 BF846115 AL525361 |
| 400245 | 12188_1 | X79449 BC017853 AL121035 BF196384 AW119044 AI028023 AW451110 |
| | | AI971911 AW015069 AI079170 AI376367 AI264113 AA829646 AA737579 AA449679 AA740864 |
| | | NM_001111 U18121 AL567297 BG773801 BF973874 AV687104 AA527579 AA843525 |
| | | BE706355 AI074589 AI523475 BE890249 AW406263 BE074258 AV729485 BF809610 |
| | | BG058619 AA677244 BE179838 AA622264 AI460106 AA740411 AI499168 AI078223 AI682923 |
| | | BE696559 AW375385 AA788739 BG984978 Z40874 T17054 F09669 AW844043 U10439 |
| | | BI711870 AW245957 AU158567 AA679305 AA679316 W72510 AI346029 BG059762 AW251062 |
| | | AA132373 AI925621 AI860230 AI340172 AW192891 AI707980 AI094937 AI042115 AI200901 |
| | | BE328452 AA644678 AA551209 BE351065 AA970761 N68609 AW002028 AA160826 AI422774 |
| | | AW873114 AW073597 AW664483 AI218710 AW020550 AW190607 AI984545 AI871921 |
| | | AI333970 AI452887 AI818335 AA398655 AI554424 AI274187 BE465703 AW512940 AW241366 |
| | | AI923954 AA576649 AW168294 AA813181 AA912168 AI049738 AW514073 AA548255 AI569630 |
| | | BE710031 AA244182 AI341697 AA563904 AI537990 AW517908 AW172943 Z39498 AI750294 |
| | | AW150414 AI253293 BE825720 T31860 AW150775 D20310 AA150892 AU133933 BE781148 |
| | | AL038957 BF910979 AA352297 BG988142 AW372175 BF229106 AW866705 BE093482 BG990396 |
| | | AI499917 AA054452 H05484 AI828502 BM467331 AU140570 AL135417 BF947202 AW391926 |
| | | BE813418 BF998473 T92021 BI021048 BM048783 AW501366 AW501342 AW501549 BE939021 |
| | | BE707147 BE160974 BE305207 N49011 AA947119 AA678801 BE536876 AW897428 BG329648 |
| | | BG818540 BE542344 BI919250 BI253018 AW130996 BE074249 BE895428 BI034862 BE083277 |
| | | BF952166 |
| 429978 | 35194_2 | BE738425 BE738323 BM126944 AW629678 AW265195 AI916735 AI394255 AI573090 |
| | | AI354442 AW612857 AI339558 AI919424 AI377532 AI354441 AI308821 AA772275 AW055215 |
| | | AI589705 AI336532 AA806547 AV682125 H93575 AW071172 AW769904 AI863985 AW265018 |
| | | AW196655 D79662 BE042393 N75017 AW014741 C75509 BE748621 H92431 AW079261 |
| | | AW901780 AA329482 AW960115 BI260621 AI767525 R31663 BI918664 AW963196 C06195 |
| | | AI678018 |
| 400213 | 6901_3 | D63478 NM_014847 BG756716 BG163437 BM463199 BG403203 AA401857 |
| | | AA368774 AW408251 AL523107 BG026453 BF674703 AA604407 N29680 AA648135 AI288204 |
| | | AI288200 AA594851 AI969057 BF033736 BI334216 BE925408 AI557055 BF328153 AI885038 |
| | | AV743562 AW152023 AI678636 AI990677 AA911648 AI184369 AI830103 AA259120 AI924057 |
| | | BG152397 AI695208 H71871 BI259526 T18975 BI259531 BF229650 |
| 447349 | 1063443_1 | BE743847 AW809603 BM469626 AI375546 |
| 400203 | 11774_1 | NM_002794 D26599 BM469989 BF305151 BG821966 BI089030 AW007738 |
| | | BI222910 BM049422 BG028749 AI189162 AI831230 AW131497 BM272215 BE791105 AW778828 |
| | | AA479594 AA480133 AA131997 AA284572 AA453009 BF928258 AA152127 AA393918 |
| | | BF765307 BE247542 BF934697 BF341798 BE253409 AA470620 AI828932 AW379902 AV762678 |
| | | AV741784 AV760892 AI025755 AA878562 AA630630 AA761708 AA862518 AA865831 AA862947 |
| | | N53065 AA131821 AA293499 N23342 N26856 AI147346 AW951549 AA772963 BE245986 |
| | | BG208493 AI831666 BG474873 BI023168 AU149647 BG197069 BG191102 BF304178 BE536135 |
| | | AA706900 AA443583 AI002710 AW276192 AU149842 BG214797 BG198193 AW197923 AW627799 |
| | | T98663 BG194788 BG214656 BF345258 BG716363 AI066528 BI546220 AA393815 AA132004 |
| | | AA353826 H97858 BG187823 BF841463 AI351714 AV735966 BG196439 BG216840 BG198438 |
| | | BG400762 |
| 456031 | 6000_2 | BG469257 BM042651 AW772237 AI858055 AW043820 AI804955 AW028151 AI887909 |
| | | AI582191 AI798900 AI299745 BF058971 BE504708 AW001777 BF000316 AW001248 AI336325 |
| | | AI805058 AI923922 BM054790 AI949719 AI741492 AI684429 AI276119 AI269740 AI183994 |
| | | AI719036 AA480650 AW263563 AI971175 AI347141 BF973966 AA480684 AI097473 AI580934 |
| | | AI200633 BF058119 AW672804 AI348656 BF732192 AW517972 AI262960 M62028 N42455 |
| | | BM050656 AA595104 AI074858 AA627913 AI304384 AW900999 AA365138 N66329 |
| | | AI648474 AA642939 AI474817 AI054319 W25028 AA863058 BG469176 AA321296 BE778988 |
| | | AI262696 BF026236 AW205513 AW594574 AA922438 W68465 BF684267 BF591011 |
| | | AA367149 BG896411 AW243042 BI335118 BF915906 BF915905 BG338858 AI813670 |
| | | AW819595 AW243178 AW819644 AW136076 AI810972 BF476651 AW515899 BE936236 |
| | | BE905314 AI242330 |
| 406685 | 0_0 | M18728 |
| 458748 | 701190_1 | AI381530 BG942599 AW270510 AW265104 |
| 415989 | 10194_1 | BC013389 BC017398 AI023543 AA191424 AI267700 AI469633 AW958465 AW953397 |
| | | AA172056 BE940298 BF909208 BF909980 BF095153 BG285837 AI720344 BF541715 |
| | | AA355086 AA172236 |
| 441128 | 20932_1 | BC014072 BE328850 AI356567 AI148171 AI022165 BG149661 BF000671 AA233101 |
| | | AA573721 AA447991 AW016855 AI005068 AA554071 BF478215 AA906902 AW014761 |
| | | BE905651 BE512923 BM047129 AA243852 AA232991 AA127550 AA127551 AA570256 |
| | | AI473237 BF033706 N90525 AW973623 AI359627 BG674574 BE903322 |
| 430677 | 11749_1 | NM_001943 Z26317 BG750290 BM043721 AW361908 BG494570 AW996792 |
| | | BF915903 AW753487 BI335109 BE018413 BE874074 BF333555 BI016581 BI014002 |
| | | BI015917 BE731644 BE927177 BE697899 BF999996 BF929423 BG951608 AW858747 |
| | | AW858755 AW858750 AW858749 AW858751 AW363742 D58979 BE003946 AW858999 |
| | | BG951830 AW859003 BF738953 AW369461 AW363740 AW998641 BG978975 BG015802 |
| | | AW991316 |
| 427239 | 20459_2 | AL532360 BE794750 AA582906 AI015067 AW271034 BG271636 AW075177 AW071374 |
| | | AI345565 AI307208 BE138953 BE049086 AI334881 AW075006 AW075181 AA464019 |
| | | AW302733 AW075100 AW073433 AI802854 AI334909 AI802853 AI345036 AI348921 |
| | | AI340734 AI307478 AI251289 AW302327 AW072520 AI312145 AW073656 AW072513 |
| | | AW071289 AI307559 AA876186 T29587 AI307493 AI255068 AI252868 AI252839 |
| | | AW074809 AI252926 AI252160 AI251662 AI251262 AI610913 AI270787 AI270156 |
| | | AI252075 AW073469 AW072901 AW072496 AW071420 AI305762 AI254764 AI802837 |
| | | AI251264 AW073049 AW071311 AI340643 BE138965 BE138502 AW073456 |
| | | AI334733 AI054335 BE139260 AI054302 AI054060 AI054057 AI053722 AI289711 |
| | | BE139228 AW470478 AW271039 AW302085 BE041872 AI254494 AI271496 AI252427 |
| | | BF718773 BF718645 AW074866 BE857822 |
| 429228 | 215430_1 | BG676155 BM009591 AI479075 AI025794 AI017967 AA448270 BE466812 AA853422 |
| | | AI392649 BG952034 AA513384 BF840124 BE714620 AW969605 AI553633 |
| 451807 | 17758_2 | BM479185 AL552795 AL577722 BF038888 BM127617 BF510346 AW450652 AA865478 |
| | | AW449519 BM127314 AI806539 AW449522 AA993634 AI827626 AA904788 |
| 411825 | 7891_1 | AK000695 AK000489 BC001688 BG235988 AW006329 AI887644 AI207230 AI148213 |
| | | AI304333 AI634653 AW662636 AI281247 AA946921 AA424487 BE272330 AI830588 |
| | | AA159183 AA977141 BG231801 AA631793 AA975194 BF817537 AA477798 BI906631 |
| | | AW083424 AA625199 NM_017767 AK000334 BF964048 AW815634 AL573992 |
| | | AA430612 AA928390 AA464447 AW340827 AA424290 AI927759 BG951502 AW881353 BI765535 |
| 428342 | 6712_1 | AK056315 AI015524 AA724079 BI713619 AI377728 AW293682 AI928140 AI092404 AI085630 |
| | | AA731340 BM469629 AW968804 AA425658 AA769094 BF446026 AW118719 AI332765 |
| | | AW500888 AW576556 AI859571 AW499664 AW614573 AW629495 AW505314 W74704 |
| | | AI356361 AI923640 AW070509 AI521500 AL042095 AA609309 AA761319 AI381489 |
| | | H45700 AA761333 AW265424 AA909524 AA635311 AA649040 AI392620 Z40708 |
| | | AI985564 AW263513 AA913892 AI693486 AW263502 AI806164 AW291137 BI061872 |
| | | BI059498 AA134476 AW084888 AA036967 AW370823 T55263 BI002756 AA489664 |
| | | BF827261 W74741 BF963166 |
| 448993 | 9350_1 | BE734790 BE797215 BG759420 AW407710 BG325963 BI918794 BE278238 BI199200 BI518986 |
| | | BI253338 BI262043 AW340858 BF375322 AI982791 AI567270 BF095628 BF095497 AI813332 |
| | | BF766170 BF431658 R69173 AA454908 AA394063 AA346417 AA346416 D20284 AA053597 |
| | | BI765427 BI819610 BM148877 AA293504 AA427705 AA399460 AA454833 AA292343 AA730380 |

Pkey: Unique Eos probeset identifier number
CAT number: Gene cluster number
Accession: Genbank accession numbers

TABLE 7C

| Pkey | Ref | Strand | Nt_position |
|---|---|---|---|
| 401131 | 8699812 | Minus | 94802-94987, 95804-95887, |
| | | | 96323-96487, 9759 |
| 402496 | 9797769 | Minus | 8615-9103 |
| 401451 | 6634068 | Minus | 119926-121272 |
| 404440 | 7528051 | Plus | 80430-81581 |
| 404996 | 6007890 | Plus | 37999-38145, 38652-38998, |
| | | | 39727-39872, 4055 |
| 405770 | 2735037 | Plus | 61057-62075 |

Pkey: Unique number corresponding to an Eos probeset
Ref: Sequence source. The 7 digit numbers in this column are Genbank Identifier (GI) numbers. “Dunham I. et at.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 8A
About 330 genes upregulated in lung metastases to the brain relative to primary lung tumors

| Pkey | ExAccn | UniGeneID | UniGene Title | R1 | SEQ ID NO(s): |
|---|---|---|---|---|---|
| 425751 | T19239 | Hs.1940 | crystallin, alpha B | 23.50 | 1993 |
| 411305 | BE241596 | Hs.69547 | myelin basic protein | 22.59 | 546 |
| 424481 | R19453 | Hs.1787 | proteolipid protein 1 (Pelizaeus-Merzbac | 20.10 | 1851 |
| 407100 | R29657 | | gb:F1-1179D 22 week old human fetal live | 12.12 | 122 |
| 454034 | NM_000691 | Hs.575 | aldehyde dehydrogenase 3 family, member | 7.30 | 4462 5792 |
| 441899 | AI372588 | Hs.8022 | TU3A protein | 6.99 | 3378 |
| 440747 | AW297226 | Hs.137840 | ESTs, Moderately similar to SIX4_HUMAN H | 6.50 | 3316 |
| 445519 | AI635202 | Hs.170132 | hypothetical protein FLJ22494 | 6.41 | 3681 |
| 456940 | H46986 | Hs.31861 | ESTs | 6.04 | 4569 |
| 424378 | W28020 | Hs.167988 | neural cell adhesion molecule 1 | 5.89 | 1837 |
| 412754 | AW160375 | Hs.74565 | amyloid beta (A4) precursor-like protein | 5.76 | 666 |
| 440889 | R76274 | Hs.28507 | ESTs | 5.67 | 3320 |
| 415279 | F04237 | Hs.1447 | glial fibrillary acidic protein | 5.61 | 923 |
| 449383 | AW444712 | Hs.196573 | ESTs | 5.53 | 4060 |
| 424458 | M29273 | Hs.1780 | myelin associated glycoprotein | 5.40 | 1848 5213 |
| 419078 | M93119 | Hs.89584 | insulinoma-associated 1 | 5.36 | 1272 5036 |
| 409327 | L41162 | Hs.53563 | collagen, type IX, alpha 3 | 5.10 | 361 4822 |
| 425988 | BE045897 | Hs.53985 | ESTs, Weakly similar to I38022 hypotheti | 5.05 | 2016 |
| 412708 | R26830 | Hs.106137 | ESTs, Weakly similar to CGHU7L collagen | 4.97 | 661 |
| 405499 | | | NM_020638*:Homo sapiens fibroblast growt | 4.75 | 4737 71 |
| 426784 | U03749 | Hs.172216 | chromogranin A (parathyroid secretory pr | 4.75 | 2098 5319 |
| 454293 | H49739 | Hs.134013 | ESTs, Moderately similar to HK61_HUMAN H | 4.72 | 4475 |
| 406536 | | | Target Exon | 4.63 | |
| 424140 | Z48051 | Hs.141308 | myelin oligodendrocyte glycoprotein | 4.59 | 1809 5200 |
| 431846 | BE019924 | Hs.271580 | uroplakin 1B | 4.57 | 2605 |
| 422656 | AI870435 | Hs.1569 | LIM homeobox protein 2 | 4.52 | 1668 |
| 419875 | AA853410 | Hs.93557 | proenkephalin | 4.51 | 1365 |
| 435504 | W26415 | | gb:29c7 Human retina cDNA randomly prime | 4.50 | 2921 |
| 435652 | N32388 | Hs.334370 | uncharacterized hypothalamus protein HBE | 4.49 | 2935 |
| 404608 | | | Homo sapiens cDNA FLJ11027 fis, clone PL | 4.47 | |
| 440700 | AW952281 | Hs.296184 | guanine nucleotide binding protein (G pr | 4.47 | 3313 |
| 409788 | AW502154 | Hs.57760 | hypothetical protein FLJ23119; KIAA1790 | 4.46 | 406 |
| 404502 | | | Target Exon | 4.43 | |
| 429401 | AW296102 | Hs.99272 | ESTs, Weakly similar to S32567 A4 protei | 4.41 | 2364 |
| 425855 | AF135025 | Hs.159679 | kallikrein 12 | 4.37 | 2006 5280 |
| 414215 | BE262191 | Hs.142003 | ESTs | 4.36 | 802 |
| 431934 | AB031481 | Hs.272214 | STG protein | 4.32 | 2619 5496 |
| 432351 | AI270313 | Hs.127762 | hypothetical protein MGC12982 | 4.31 | 2659 |
| 423011 | NM_000683 | Hs.123022 | adrenergic, alpha-2C-, receptor | 4.31 | 1710 5166 |
| 403233 | | | C2001664*:gi\|9944235\|emb\|CAC05418.1\|(AJ | 4.28 | |
| 402000 | | | C17000761:gi\|4506525\|ref\|NP_003952.1\|rh | 4.25 | |
| 448677 | AI560769 | | ESTs | 4.22 | 3996 |
| 433447 | U29195 | Hs.3281 | neuronal pentraxin II | 4.22 | 2764 5536 |
| 410028 | AW576454 | | ESTs | 4.19 | 433 |
| 404672 | | | Target Exon | 4.11 | |
| 431083 | AF110400 | Hs.249200 | fibroblast growth factor 19 | 4.07 | 2530 5469 |
| 424922 | BE386547 | Hs.217112 | hypothetical protein MGC10825 | 4.06 | 1900 |
| 416072 | AL110370 | Hs.79000 | growth associated protein 43 | 4.00 | 970 |
| 414323 | NM_014759 | Hs.334688 | KIAA0273 gene product | 3.99 | 4918 811 |
| 415314 | N88802 | Hs.5422 | glycoprotein M6B | 3.97 | 925 |
| 416340 | N31772 | Hs.79226 | fasciculation and elongation protein zet | 3.97 | 988 |
| 452023 | AB032999 | Hs.27566 | KIAA1173 protein | 3.96 | 4264 5753 |
| 407450 | AJ006520 | | gb:Homo sapiens mRNA for m1 muscarinic a | 3.94 | 163 4771 |
| 426344 | H41821 | Hs.322469 | transcriptional activator of the c-fos p | 3.92 | 2055 |
| 436551 | AI888055 | Hs.34198 | ESTs | 3.91 | 2991 |
| 422756 | AA441787 | Hs.119689 | glycoprotein hormones, alpha polypeptide | 3.89 | 1679 |
| 425690 | AW862317 | Hs.47974 | ESTs, Moderately similar to JC4969 pig-c | 3.88 | 1988 |
| 427207 | AF012131 | Hs.173984 | T-box 1 | 3.88 | 2131 5333 |
| 425057 | AA826434 | Hs.1619 | achaete-scute complex (Drosophila) homol | 3.81 | 1915 |
| 420320 | AB002361 | Hs.96633 | KIAA0363 protein | 3.78 | 1411 5073 |
| 406121 | | | Target Exon | 3.78 | |
| 402356 | | | C19000583*:gi\|7295202\|gb\|AAF50525.1\| (AE | 3.77 | |
| 447028 | AI973128 | Hs.167257 | brain link protein-1 | 3.77 | 3813 |
| 414063 | H26904 | Hs.75736 | apolipoprotein D | 3.76 | 783 |
| 423073 | BE252922 | Hs.123119 | MAD (mothers against decapentaplegic, Dr | 3.75 | 1715 |
| 408748 | J05500 | Hs.47431 | spectrin, beta, erythrocytic (includes s | 3.73 | 302 4806 |
| 422357 | AF016272 | Hs.115418 | cadherin 16, KSP-cadherin | 3.73 | 1634 5148 |
| 449227 | AJ403106 | Hs.121824 | ESTs | 3.70 | 4048 |
| 425450 | U14755 | Hs.157449 | LIM homeobox protein 1 | 3.69 | 1965 5264 |
| 419555 | AA244416 | | gb:nc07d11.s1 NCI_CGAP_Pr1 Homo sapiens | 3.69 | 1331 |
| 438944 | AA302517 | Hs.92732 | KIAA1444 protein | 3.68 | 3179 |
| 433932 | AW954599 | Hs.169330 | neuronal protein | 3.66 | 2800 |
| 413946 | BE185066 | Hs.2055 | gb:MR1-HT0709-100500-002-c09 HT0709 Homo | 3.65 | 767 |
| 406273 | | | peptidylglycine alpha-amidating monooxyg | 3.62 | |
| 427993 | AA418483 | Hs.104806 | ESTs | 3.62 | 2212 |
| 438743 | AW664029 | Hs.129496 | ESTs | 3.57 | 3163 |
| 447868 | AI434802 | Hs.196071 | ESTs | 3.56 | 3916 |
| 450875 | AK000724 | | karyopherin alpha 6 (importin alpha 7) | 3.55 | 4172 |
| 457842 | AJ251759 | Hs.283846 | Homo sapiens GNAS1 antisense transcript | 3.54 | 4602 |
| 404033 | | | C5000413*:gi\|202800\|gb\|AAA40703.1\| (M647 | 3.54 | |
| 423473 | H49104 | Hs.129888 | hypothetical protein FLJ14768 | 3.53 | 1750 |
| 446639 | AI016826 | Hs.342148 | ESTs | 3.51 | 3774 |
| 421074 | AI690321 | Hs.203845 | two pore potassium channel KT3.3 | 3.51 | 1478 |
| 422440 | NM_004812 | Hs.116724 | aldo-keto reductase family 1, member B10 | 3.50 | 1647 5151 |
| 441030 | AW204139 | Hs.174424 | ESTs, Weakly similar to para-aminohippur | 3.50 | 3328 |
| 421771 | NM_001224 | Hs.108131 | caspase 2, apoptosis-related cysteine pr | 3.50 | 1561 5121 |
| 423420 | AI571364 | Hs.128382 | Homo sapiens mRNA; cDNA DKFZp76111224 (f | 3.48 | 1743 |
| 401980 | | | Target Exon | 3.46 | |
| 400739 | | | ENSP00000240971:Voltage-gated potassium | 3.45 | |
| 409915 | AA701921 | Hs.188436 | ESTs | 3.44 | 417 |
| 402560 | | | NM_006610*:Homo sapiens mannan-binding I | 3.43 | 36 4705 |
| 406939 | M34515 | | gb:Human omega light chain protein 14.1 | 3.42 | 112 4758 |
| 401670 | | | Target Exon | 3.41 | |
| 420485 | AF218586 | Hs.288835 | cell death-inducing DFFA-like effector b | 3.40 | 1424 5077 |
| 414408 | BE294783 | Hs.279497 | hypothetical protein MGC4638 | 3.39 | 823 |
| 435060 | AI422719 | Hs.120873 | ESTs, Weakly similar to fork head like p | 3.39 | 2891 |
| 402206 | | | Target Exon | 3.37 | |
| 435763 | AI243929 | | ESTs | 3.37 | 2941 |
| 400307 | AF005081 | | Homo sapiens skin-specific protein (xp32 | 3.37 | 11 4684 |
| 429897 | AA460370 | Hs.103189 | lipopolysaccharide specific response-68 | 3.34 | 2422 |
| 413333 | M74028 | Hs.75297 | fibroblast growth factor 1 (acidic) | 3.33 | 712 |
| 410939 | AW811324 | | gb:IL3-ST0141-131099-017-A02 ST0141 Homo | 3.32 | 522 |
| 433657 | AI244368 | Hs.1787 | PH domain containing protein in retina 1 | 3.31 | 2777 |
| 409702 | AI752244 | | eukaryotic translation elongation factor | 3.31 | 395 |
| 406131 | | | Target Exon | 3.31 | |
| 455973 | BE175424 | | gb:RC4-HT0578-170300-012-d01 HT0578 Homo | 3.30 | 4524 |
| 453169 | AB037815 | Hs.32156 | KIAA1394 protein | 3.30 | 4382 5771 |
| 415796 | R87548 | Hs.78854 | ATPase, Na? transporting, beta 2 polypep | 3.29 | 954 |
| 426039 | BE265133 | Hs.217493 | annexin A2 | 3.28 | 2021 |
| 419705 | AW368634 | Hs.154331 | ESTs | 3.27 | 1351 |
| 416916 | AW749469 | Hs.61784 | hypothetical protein FLJ14451 | 3.25 | 1036 |
| 405326 | | | Target Exon | 3.25 | |
| 420783 | AI659838 | Hs.99923 | lectin, galactoside-binding, soluble, 7 | 3.24 | 1453 |
| 418064 | BE387287 | Hs.83384 | S100 calcium-binding protein, beta (neur | 3.24 | 1155 |
| 423869 | BE409301 | Hs.134012 | C1q-related factor | 3.23 | 1782 |
| 436420 | AA443966 | Hs.31595 | ESTs | 3.23 | 2984 |
| 444039 | NM_016348 | Hs.10235 | chromosome 5 open reading frame 4 | 3.22 | 3569 5633 |
| 423897 | AB033062 | Hs.134970 | DKFZP434N178 protein | 3.22 | 1784 5189 |
| 452240 | AI591147 | Hs.61232 | ESTs | 3.21 | 4286 |
| 407208 | T10695 | | gb:hbc970 Human pancreatic islet Homo sa | 3.21 | 137 |
| 435538 | AB011540 | Hs.4930 | low density lipoprotein receptor-related | 3.21 | 2924 5565 |
| 454188 | AW177809 | | gb:IL3-HT0059-180899-007-C12 HT0059 Homo | 3.20 | 4471 |
| 408452 | AA054683 | Hs.192455 | ESTs, Weakly similar to ALU7_HUMAN ALU S | 3.20 | 268 |
| 423801 | NM_015071 | Hs.132942 | GTPase regulator associated with the foc | 3.19 | 1776 5188 |
| 458904 | NM_012155 | Hs.24178 | microtubule-associated protein like echi | 3.19 | 4645 5817 |
| 440274 | R24595 | Hs.7122 | scrapie responsive protein 1 | 3.18 | 3275 |
| 414961 | U27266 | Hs.927 | myosin-binding protein H | 3.18 | 4935 895 |
| 421511 | AA488940 | Hs.105216 | hypothetical protein FLJ11125 | 3.17 | 1530 |
| 401478 | | | Target Exon | 3.17 | |
| 451111 | W96141 | Hs.220687 | ESTs | 3.17 | 4195 |
| 409275 | F05689 | Hs.269257 | ESTs | 3.17 | 359 |
| 440109 | AK001138 | Hs.333149 | hypothetical protein FLJ10276 | 3.16 | 3264 5602 |
| 406907 | Z25427 | | gb:H. sapiens protein-serine/threonine ki | 3.16 | 107 4753 |
| 413166 | BE068507 | | gb:MR1-BT0371-070500-010-c11 BT0371 Homo | 3.15 | 697 |
| 455984 | BE177442 | | gb:RC1-HT0595-200400-012-f01 HT0595 Homo | 3.14 | 4525 |
| 414463 | T69078 | Hs.76177 | alpha-1-microglobulin/bikunin precursor | 3.13 | 832 |
| 406535 | | | Target Exon | 3.13 | |
| 430294 | AI538226 | Hs.32976 | guanine nucleotide binding protein 4 | 3.13 | 2463 |
| 423134 | AJ012582 | Hs.124161 | hyperpolarization activated cyclic nucle | 3.13 | 1720 5170 |
| 446598 | AW250546 | Hs.59317 | gb:2821774.5prime NIH_MGC_7 Homo sapiens | 3.12 | 3769 |
| 422637 | AA399024 | Hs.118836 | myoglobin | 3.12 | 1666 |
| 453439 | AI572438 | Hs.32976 | guanine nucleotide binding protein 4 | 3.11 | 4406 |
| 441251 | BE010471 | Hs.257747 | ESTs | 3.10 | 3340 |
| 454453 | AW752781 | | hypothetical protein FLJ12614 similar to | 3.10 | 4485 |
| 413570 | BE158651 | Hs.294022 | hypothetical protein FLJI4950 | 3.09 | 735 |
| 424098 | AF077374 | Hs.139322 | small proline-rich protein 3 | 3.08 | 1804 5199 |
| 403437 | | | C3001181*:gi\|12052997\|emb\|CAB66673.1\| (A | 3.08 | |
| 458579 | AI241355 | Hs.334604 | Homo sapiens mRNA for KIAA1870 protein, | 3.08 | 4628 |
| 453991 | AW014915 | Hs.273741 | ESTs | 3.07 | 4459 |
| 431912 | AI660552 | | ESTs, Weakly similar to A56154 AbI subst | 3.06 | 2615 |
| 401826 | | | Target Exon | 3.06 | |
| 410022 | AA079727 | | gb:zm20e09.r1 Stratagene pancreas (93720 | 3.05 | 430 |
| 420262 | AW964073 | Hs.58362 | hypothetical protein FLJ12681 | 3.05 | 1403 |
| 431917 | D16181 | Hs.2868 | peripheral myelin protein 2 | 3.04 | 2616 5494 |
| 448389 | AW188950 | Hs.345838 | ESTs | 3.04 | 3962 |
| 428830 | AC002115 | Hs.194061 | ets variant gene 2 | 3.03 | 2307 5380 |
| 447414 | D82343 | Hs.74376 | neuroblastoma (nerve tissue) protein | 3.02 | 3859 5687 |
| 407168 | R45175 | Hs.117183 | ESTs | 3.02 | 131 |
| 422010 | AA302049 | Hs.31181 | Homo sapiens cDNA: FLJ23230 fis, clone C | 3.02 | 1593 |
| 448191 | NM_005881 | Hs.20644 | branched chain alpha-ketoacid dehydrogen | 3.02 | 3950 5708 |
| 458963 | AI701393 | Hs.278728 | Rad and Gem-related 2 (rat homolog) | 3.01 | 4647 |
| 402409 | | | Target Exon | 3.00 | |
| 421048 | AI821593 | Hs.186669 | ESTs | 3.00 | 1476 |
| 417697 | R09609 | Hs.193118 | ESTs | 3.00 | 1114 |
| 433951 | AW274301 | Hs.165384 | ESTs | 3.00 | 2801 |
| 411908 | L27943 | Hs.72924 | cytidine deaminase | 3.00 | 4869 585 |
| 444828 | AI198680 | | gb:qf51f02.x1 Soares_testis_NHT Homo sap | 2.99 | 3632 |
| 451619 | AA018854 | | glutathione peroxidase 3 (plasma) | 2.98 | 4234 |
| 423515 | AA327017 | Hs.176594 | ESTs | 2.96 | 1754 |
| 441134 | W29092 | Hs.346950 | cellular retinoic acid-binding protein 1 | 2.95 | 3335 |
| 446204 | AI279809 | Hs.150019 | ESTs | 2.95 | 3735 |
| 431677 | AK000496 | Hs.306989 | hypothetical protein FLJ20489 | 2.94 | 2582 5485 |
| 434859 | BE255080 | Hs.299315 | collapsin response mediator protein-5; C | 2.94 | 2874 |
| 402220 | | | C19000596*:gi\|6755546\|ref\|NP_036107.1\|s | 2.94 | |
| 453629 | BE179903 | | gb:IL3-HT0618-110500-133-F09 HT0618 Homo | 2.93 | 4420 |
| 443219 | AI354669 | Hs.187461 | ESTs, Weakly similar to C29149 proline-r | 2.93 | 3509 |
| 413248 | T64858 | Hs.26966 | hypothetical protein DKFZp547J036 | 2.93 | 703 |
| 434067 | H18913 | Hs.124023 | Homo sapiens cDNA FLJ14218 fis, clone NT | 2.93 | 2812 |
| 400840 | | | Target Exon | 2.93 | |
| 404381 | | | Target Exon | 2.93 | |
| 424340 | AA339036 | Hs.7033 | ESTs | 2.93 | 1832 |
| 415651 | AI207162 | Hs.3815 | stathmin-like-protein RB3 | 2.91 | 942 |
| 447103 | AI361877 | Hs.270564 | ESTs, Weakly similar to pro alpha 1(I) c | 2.91 | 3821 |
| 453120 | AA292891 | Hs.31773 | pregnancy-induced growth inhibitor | 2.91 | 4375 |
| 456454 | T90789 | Hs.94308 | RAB35, member RAS oncogene family | 2.90 | 4543 |
| 424306 | AI863322 | Hs.192985 | ESTs, Weakly similar to KIAA0227 [H. sapi | 2.89 | 1826 |
| 449792 | AI568508 | Hs.239444 | ESTs, Weakly similar to JC5963 stable tu | 2.88 | 4084 |
| 408726 | BE046452 | Hs.255739 | ESTs, Weakly similar to Z205_HUMAN ZINC | 2.88 | 297 |
| 443906 | AA348031 | Hs.7913 | ESTs | 2.87 | 3562 |
| 418828 | AF020774 | Hs.88844 | Homo sapiens hair and skin epidermal-typ | 2.87 | 1246 |
| 425287 | R88249 | Hs.155524 | peanut (Drosophila)-like 2 | 2.86 | 1948 |
| 420767 | AF072711 | Hs.99918 | carboxyl ester lipase (bile salt-stimula | 2.86 | 1452 5086 |
| 417390 | AA196552 | Hs.85852 | hypothetical protein MGC3169 | 2.85 | 1092 |
| 437595 | AI028309 | Hs.114246 | ESTs | 2.85 | 3073 |
| 419663 | AA394208 | Hs.92198 | calcium-regulated heat-stable protein (2 | 2.84 | 1345 |
| 406320 | | | Target Exon | 2.84 | |
| 452263 | AA025116 | Hs.33333 | ESTs | 2.83 | 4291 |
| 433472 | AI541246 | Hs.3343 | phosphoglycerate dehydrogenase | 2.83 | 2765 |
| 420751 | J03019 | Hs.99913 | adrenergic, beta-1-, receptor | 2.83 | 1450 5085 |
| 454066 | X00356 | Hs.37058 | calcitonin/calcitonin-related polypeptid | 2.82 | 4467 5794 |
| 437926 | BE383605 | Hs.300816 | small GTP-binding protein | 2.81 | 3107 |
| 406881 | D16154 | | gb:Human gene for cytochrome P-450c11, e | 2.81 | 106 |
| 437745 | AI381515 | Hs.158381 | ESTs | 2.80 | 3086 |
| 425939 | AK001527 | Hs.163953 | hypothetical protein FLJ10665 | 2.80 | 2012 5282 |
| 407375 | AA091354 | | gb:II0815.seq.F Human fetal heart, Lambd | 2.80 | 158 |
| 427344 | NM_000869 | Hs.2142 | 5-hydroxytryptamine (serotonin) receptor | 2.79 | 2145 5336 |
| 430393 | BE185030 | Hs.241305 | estrogen-responsive B box protein | 2.79 | 2480 |
| 434607 | R70848 | Hs.163568 | ESTs | 2.78 | 2851 |
| 401940 | | | Target Exon | 2.78 | |
| 427693 | BE546832 | Hs.180370 | cofilin 1 (non-muscle) | 2.78 | 2182 |
| 429487 | M13077 | Hs.284255 | alkaline phosphatase, placental (Regan i | 2.77 | 2376 5409 |
| 413662 | BE155866 | Hs.25522 | KIAA1808 protein | 2.77 | 746 |
| 451179 | W05469 | Hs.31818 | ESTs | 2.77 | 4199 |
| 444643 | AW450739 | Hs.28077 | GDP-mannose pyrophosphorylase B | 2.77 | 3615 |
| 401504 | | | Target Exon | 2.77 | |
| 407570 | Z19002 | Hs.37096 | zinc finger protein 145 (Kruppel-like, e | 2.77 | 169 4775 |
| 453830 | AA534296 | Hs.20953 | ESTs | 2.76 | 4434 |
| 428342 | AI739168 | | Homo sapiens cDNA FLJ13458 fis, clone PL | 2.76 | 2244 |
| 440548 | AL117408 | Hs.7274 | DKFZP434P1750 protein | 2.76 | 3299 5609 |
| 433879 | AA613312 | Hs.291798 | ESTs | 2.76 | 2795 |
| 416530 | U62801 | Hs.79361 | kallikrein 6 (neurosin, zyme) | 2.76 | 1001 4963 |
| 404224 | | | Target Exon | 2.75 | |
| 409836 | AA077964 | | gb:7H12A01 Chromosome 7 HeLa cDNA Librar | 2.75 | 408 |
| 443665 | AV646058 | Hs.282325 | ESTs | 2.74 | 3535 |
| 431932 | AB015349 | Hs.22361 | Homo sapiens HRIHFB2063 mRNA, partial cd | 2.74 | 2617 5495 |
| 429545 | AI824164 | | lymphocyte antigen 6 complex, locus E | 2.74 | 2386 |
| 406979 | M85247 | | gb:H. sapiens dopamine D1A receptor gene, | 2.74 | 116 |
| 444506 | AI159863 | Hs.143713 | ESTs | 2.74 | 3603 |
| 419727 | AW160796 | Hs.92700 | DKFZP564O243 protein | 2.74 | 1353 |
| 409557 | BE182896 | Hs.3686 | ESTs | 2.73 | 384 |
| 452309 | AW206836 | Hs.224491 | ESTs | 2.73 | 4295 |
| 435814 | AW615179 | Hs.152870 | ESTs | 2.73 | 2947 |
| 442926 | AW014374 | Hs.144849 | ESTs | 2.73 | 3483 |
| 454360 | L78207 | Hs.54470 | ATP-binding cassette, sub-family C (CFTR | 2.72 | 4478 5796 |
| 425547 | AA359181 | | gb:EST68183 Fetal lung II Homo sapiens c | 2.72 | 1977 |
| 427587 | BE348244 | Hs.284239 | ESTs, Weakly similar to I78885 serine/th | 2.72 | 2170 |
| 421871 | AK001416 | Hs.306122 | glycoprotein, synaptic 2 | 2.71 | 1573 |
| 448045 | AJ297436 | Hs.20166 | prostate stem cell antigen | 2.71 | 3937 5706 |
| 442257 | AW503831 | | Human EST clone 25267 mariner transposon | 2.71 | 3407 |
| 441834 | AL138034 | Hs.7979 | KIAA0736 gene product | 2.71 | 3374 |
| 425211 | M18667 | Hs.1867 | progastricsin (pepsinogen C) | 2.71 | 1936 5251 |
| 447128 | AI271898 | | cyclin K | 2.70 | 3825 |
| 408115 | AB033107 | Hs.42796 | KIAAI281 protein | 2.70 | 236 4789 |
| 407394 | AF005081 | | gb:Homo sapiens skin-specific protein (x | 2.70 | 162 4770 |
| 445570 | AW239531 | Hs.102367 | HMG-box transcription factor TCF-3 | 2.70 | 3685 |
| 405060 | | | Target Exon | 2.70 | |
| 403016 | | | C21000452*:gi\|7657283\|ref\|NP_056623.1\| k | 2.68 | |
| 420037 | BE299598 | Hs.135569 | hypothetical protein FLJ14708 | 2.68 | 1376 |
| 401007 | | | ENSP00000227594*:FLJ00043 protein (Fragm | 2.67 | |
| 423942 | AF209704 | Hs.169407 | glycolipid transfer protein | 2.67 | 1785 5190 |
| 453210 | AL133161 | Hs.32360 | hypothetical protein FLJ10867 | 2.67 | 4387 5772 |
| 429259 | AA420450 | Hs.292911 | Plakophilin | 2.67 | 2344 |
| 400524 | | | Target Exon | 2.66 | |
| 453017 | R84301 | Hs.31387 | DKFZP564J0123 protein | 2.66 | 4367 |
| 414213 | BE297765 | | gb:601176246F1 NIH_MGC_17 Homo sapiens c | 2.66 | 801 |
| 416056 | H18056 | Hs.6829 | Homo sapiens mRNA for KIAA1644 protein, | 2.65 | 968 |
| 438245 | AL137688 | Hs.6132 | copine VI (neuronal) | 2.65 | 3127 |
| 425256 | BE297611 | Hs.155392 | collapsin response mediator protein 1 | 2.65 | 1945 |
| 416413 | H53930 | Hs.35354 | ESTs | 2.65 | 995 |
| 412595 | AA113749 | | gb:zn66d07.r1 Stratagene HeLa cell s3 93 | 2.65 | 650 |
| 428439 | BE467359 | Hs.24986 | ESTs | 2.65 | 2258 |
| 411830 | BE549420 | Hs.173497 | Sec23 (S. cerevisiae) homolog B | 2.64 | 581 |
| 417343 | AA197132 | Hs.231581 | myosin, heavy polypeptide 1, skeletal mu | 2.64 | 1083 |
| 447582 | BE293520 | Hs.18910 | prostate cancer overexpressed gene 1 | 2.64 | 3883 |
| 427627 | R87582 | Hs.179915 | guanine nucleotide binding protein (G pr | 2.64 | 2174 |
| 417852 | AJ250562 | Hs.82749 | transmembrane 4 superfamily member 2 | 2.63 | 1130 4994 |
| 446924 | AW134643 | | EST | 2.63 | 3801 |
| 429024 | AI652297 | Hs.119302 | complement-c1q tumor necrosis factor-rel | 2.62 | 2326 |
| 407846 | AA426202 | Hs.40403 | Cbp/p300-interacting transactivator, wit | 2.62 | 208 |
| 426328 | AW631296 | Hs.123933 | gb:hh83c09.y1 NCI_CGAP_GU1 Homo sapiens | 2.62 | 2053 |
| 400789 | | | C11001367*:gi\|1076205\|pir∥S50754 hypoth | 2.61 | |
| 402792 | | | Target Exon | 2.61 | |
| 400655 | | | Target Exon | 2.61 | |
| 401496 | | | Target Exon | 2.60 | |
| 401663 | | | Target Exon | 2.60 | |
| 431056 | BE616373 | Hs.249163 | fatty acid hydroxylase | 2.60 | 2528 |
| 407027 | U63312 | | gb:Human cosmid LL12NC01-242E1, ETV6 gen | 2.60 | 120 4763 |
| 412659 | AW753865 | Hs.74376 | olfactomedin related ER localized protei | 2.60 | 656 |
| 422927 | AW247388 | Hs.301423 | calcium binding protein 1 (calbrain) | 2.59 | 1693 |
| 409108 | AA339443 | Hs.48793 | sialyltransferase 6 (N-acetyllacosaminid | 2.59 | 334 |
| 425001 | U55184 | Hs.154145 | hypothetical protein FLJ11585 | 2.59 | 1908 5241 |
| 457131 | AC002310 | Hs.301463 | Human Chromosome 16 BAC close CIT987SK-A | 2.59 | 4579 5810 |
| 432941 | W04803 | Hs.279851 | hypothetical protein FLJ10241 | 2.59 | 2713 |
| 416889 | AW250318 | Hs.80395 | mal, T-cell differentiation protein | 2.59 | 1034 |
| 437800 | AA897636 | Hs.159366 | ESTs | 2.58 | 3092 |
| 401815 | | | Target Exon | 2.58 | |
| 423849 | AL157425 | Hs.133315 | Homo sapiens mRNA; cDNA DKFZp761J1324 (f | 2.58 | 1780 |
| 419272 | AA663904 | Hs.89862 | TNFRSF1A-associated via death domain | 2.58 | 1297 |
| 402978 | | | Target Exon | 2.57 | |
| 428931 | AA994979 | Hs.98967 | ATPase, H()-transporting, lysosomal, non | 2.57 | 2317 |
| 444613 | H29627 | Hs.79092 | hypothetical protein FLJ14427 | 2.57 | 3613 |
| 441087 | AI797588 | | ESTs | 2.57 | 3332 |
| 409870 | AW502321 | | gb:UI-HF-BR0p-ajs-e-05-0-UI.r1 NIH_MGC_5 | 2.57 | 410 |
| 438870 | AF075011 | | gb:Homo sapiens full length insert cDNA | 2.57 | 3172 |
| 439340 | AB032436 | Hs.6535 | brain-specific Na-dependent inorganic ph | 2.56 | 3200 5598 |
| 439671 | AW162840 | Hs.6641 | kinesin family member 5C | 2.56 | 3227 |
| 439285 | AL133916 | | hypothetical protein FLJ20093 | 2.56 | 3196 |
| 407561 | BE313226 | Hs.94761 | KIAA1691 protein | 2.56 | 167 |
| 423118 | AL035460 | Hs.124009 | Human DNA sequence from clone RP5-860F19 | 2.56 | 1719 5169 |
| 435406 | F26698 | Hs.4884 | calcium/calmodulin-dependent protein kin | 2.56 | 2911 |
| 442660 | AW138174 | Hs.130651 | ESTs | 2.55 | 3458 |
| 403649 | | | Target Exon | 2.55 | |
| 450493 | W93718 | Hs.166373 | nitric oxide synthase 3 (endothelial cel | 2.55 | 4140 5731 |
| 424463 | AW195353 | Hs.119903 | ESTs | 2.54 | 1850 |
| 432201 | AI538613 | Hs.298241 | Transmembrane protease, serine 3 | 2.54 | 2643 |
| 401915 | | | Target Exon | 2.54 | |
| 439796 | AI566839 | Hs.124483 | ESTs | 2.54 | 3236 |
| 433091 | Y12642 | Hs.3185 | lymphocyte antigen 6 complex, locus D | 2.54 | 2735 5527 |
| 448555 | AI536697 | Hs.159863 | ESTs | 2.54 | 3984 |
| 429712 | AW245825 | Hs.211914 | ENSP00000233627*:NADH-ubiquinone oxidore | 2.54 | 2410 |
| 455145 | AW858396 | Hs.208680 | ESTs | 2.54 | 4501 |
| 431463 | X95632 | Hs.343575 | abl-interactor 12 (SH3-containing protei | 2.53 | 2558 5477 |
| 440545 | AW183201 | Hs.190559 | ESTs | 2.53 | 3297 |
| 413865 | AA132816 | Hs.151714 | Homo sapiens mRNA for KIAA1769 protein, | 2.53 | 759 |
| 406312 | | | Target Exon | 2.53 | |
| 428366 | AI803651 | Hs.191608 | ESTs | 2.52 | 2245 |
| 417823 | R88869 | Hs.102447 | TSC-22-like | 2.52 | 1127 |
| 429211 | AF052693 | Hs.198249 | gap junction protein, beta 5 (connexin 3 | 2.52 | 2338 5394 |
| 413140 | T06607 | Hs.6846 | hypothetical protein FLJ13055 | 2.52 | 694 |
| 424342 | AF131778 | Hs.145432 | similar to PSD-95/SAP90-associated prote | 2.52 | 1833 5209 |
| 447823 | AI432628 | Hs.80887 | v-yes-1 Yamaguchi sarcoma viral related | 2.52 | 3910 |
| 404807 | | | Target Exon | 2.51 | |
| 417998 | AW967420 | | gb:EST379495 MAGE resequences, MAGJ Homo | 2.51 | 1144 |
| 426600 | NM_003378 | Hs.171014 | VGF nerve growth factor inducible | 2.51 | 2081 5312 |
| 420259 | AF004884 | Hs.96253 | calcium channel, voltage-dependent, P/Q | 2.51 | 1402 5072 |
| 402201 | | | C19000288*:gi\|7209634\|dbj\|BAA92285.1\| (A | 2.51 | |
| 451698 | Y16187 | Hs.26880 | endothelin converting enzyme-like 1 | 2.50 | 4241 5748 |
| 447786 | BE620810 | Hs.39619 | hypothetical protein LOC57333 | 2.50 | 3907 |
| 459081 | W07808 | Hs.141662 | ESTs, Weakly similar to ALU1_HUMAN ALU S | 2.50 | 4656 |
| 423063 | BE159877 | Hs.120824 | hypothetical protein FLJ21845 | 2.50 | 1714 |

Pkey: Unique Eos probeset identifier number
ExAccn: Exemplar Accession number, Genbank accession number
UniGeneID: UniGene number
UniGene Title: UniGene gene title
R1: 90th percentile of lung metastases to the brain Als divided by the 90th percentile of lung adenocarcinoma Als, where the 15th percentile of all normal body tissue Als was subtracted from the numerator and denominator.
SEQ ID NO(s): SEQ ID number(s) for nucleic acid and protein sequences associated with table entry.

TABLE 8B

| Pkey | CAT number | Accession |
|---|---|---|
| 435504 | 45952_3 | W26415 BI041755 BI040636 AA683081 BF954853 BF964120 BF848451 |
| | | BE143889 BF827761 AW899337 AW899594 BF947312 |
| 448677 | 634318_1 | AI560769 AI857497 AW151454 |
| 410028 | 958086_1 | AW576454 AW576456 AW576435 |
| 407450 | 7399_1 | BC022984 BC007740 AF385587 NM_000738 AL536991 AL537084 |
| | | AI500293 AI685922 AI821510 AI791651 AW451282 AJ006520 BI757352 |
| | | BI913662 BI197683 BI755888 BI913560 BF315022 H19732 AL566641 |
| | | AL566592 AI202576 AA531511 BF313917 BI198135 BG820557 |
| 419555 | 252042_1 | AA244401 AA244416 |
| 450875 | 10801_1 | AL041364 BE393266 AA573189 BF589066 AI623423 AI889612 H54292 |
| | | AA085863 AA669816 BE542832 BI094274 AI360690 T61853 AW081194 |
| | | AI541147 AW750358 |
| 435763 | 135600_1 | AA699940 AI243929 AA777255 |
| 400307 | 27110_2 | AF005081 BG193848 |
| 410939 | 1064852_2 | AW811332 AW811335 AW811328 AW811329 AW811339 |
| 409702 | 38388_1 | AK056951 AK026458 BI439120 BM021106 F30243 BM055214 BM054962 |
| | | BM069667 F37401 AA563621 AI752243 AI720773 AI933014 F18964 F35317 |
| | | F35258 F27772 H39537 AW445222 F19408 H28557 F30608 F31797 F30960 |
| | | BF837737 BF837688 AL551046 BI758668 BI765038 BI837440 BE392882 |
| | | BI438801 AI093511 AI752244 AI784111 BG490221 BF338840 BF338974 BG896472 |
| | | AL576843 AW966769 F25388 F37436 H28558 AI025548 AA782333 F30929 F36002 |
| | | F21229 AI720539 AA719449 F21231 F18924 AA626886 F30774 F27704 F31411 |
| | | F31127 F33381 F36153 F31793 F31138 F31966 F33901 AA298244 BI757347 |
| | | AI810201 AI692843 F29441 H51409 F21804 AW973249 F18440 F17572 F32499 |
| | | AA327152 AA534140 AI188088 F18893 F23362 AA010888 F18143 Z28500 H27651 |
| | | AI720790 F22425 H13178 H28677 F21098 F37777 F21466 F16598 F23420 |
| | | AL574723 R75610 F34035 F17845 F18560 F25902 R79117 F35534 F15713 |
| | | AI612800 F16563 F15645 F33609 F29995 BG939623 F17385 F17384 F18660 |
| | | F17922 F15523 AI093253 F18359 F31452 F00232 AI583430 BM021353 AA284108 |
| | | H27650 H29935 BE708208 AA010737 H51451 Z19399 AI678418 AI952535 F17265 |
| | | F17826 F37939 F35639 F17367 W75962 R70189 Z28755 R72106 AA335915 R75700 |
| | | R79116 W72887 AI581552 R71403 F23388 C03913 BI756149 BI116109 BF790727 |
| | | AL553994 R82966 W47487 AA456066 AW984608 BE708220 BG490537 W47419 |
| 455973 | 1561647_1 | BF352282 BE175424 BE175418 BE175383 |
| 454188 | 645555_1 | R81094 AW177908 AW177809 |
| 413166 | 1492542_1 | BE068443 BE068507 BE068731 BE068509 BE068511 BE068728 BE068636 |
| | | BE068633 BE068695 BE068730 BE068322 BE068510 BE068581 BE068518 |
| | | BE068779 BE068397 BE068515 BE068333 |
| 455984 | 1563239_1 | BE177445 BE177448 BE177440 BE177433 BE177439 BE177444 BE177442 |
| 454453 | 8582_4 | BF313069 BE879305 AW752781 AW752727 AW752559 AW752578 AW752584 |
| | | BF646118 AL545903 BF846115 AL525361 |
| 431912 | 610_10 | BI763666 BI517886 BI759051 AI688604 AI660552 BF588523 AW004785 AW295479 |
| | | BF591117 BF002672 BF064073 AA594441 AI380340 AI700219 AI659950 |
| | | AI688540 AW296326 |
| 410022 | 2576815_1 | AA824267 AA079728 AA079727 |
| 444828 | 2816194_1 | AI919288 AI824969 AI824982 AI198680 |
| 451619 | 58817_2 | AB067501 BE168197 AA477760 BF365805 AI658698 AI420374 AW006448 |
| | | BE168079 BE179882 BF352503 AW850126 AI902617 AI344041 AI805642 BG914033 |
| | | BE710337 F01902 H08649 BE180351 BE047272 R46826 AA897207 BE180115 |
| 453629 | 29513_2 | BG772130 BF928490 BI559843 BF755758 BF905496 BG718616 BG899602 |
| | | BG772217 BG719694 BI767332 AL046349 BI005797 BE179903 |
| 428342 | 6712_1 | AK056315 AI015524 AA724079 BI713619 AI377728 AW293682 AI928140 AI092404 |
| | | AI085630 AA731340 BM469629 AW968804 AA425658 AA769094 BF446026 AW118719 |
| | | AI332765 AW500888 AW576556 AI859571 AW499664 AW614573 AW629495 AW505314 |
| | | W74704 AI356361 AI923640 AW070509 AI521500 AL042095 AA609309 AA761319 AI381489 |
| | | H45700 AA761333 AW265424 AA909524 AA635311 AA649040 AI392620 Z40708 AI985564 |
| | | AW263513 AA913892 AI693486 AW263502 AI806164 AW291137 BI061872 BI059498 AA134476 |
| | | AW084888 AA036967 AW370823 T55263 BI002756 AA489664 BF827261 W74741 BF963166 |
| 409836 | 2510498_1 | AA077964 AA768182 AA077813 |
| 429545 | 934_5 | BG678726 BG708995 AI823645 AI669787 AI676005 AA456229 BF475367 BM021242 |
| | | AW129612 BF109992 AI298273 AI823860 AI804012 AI824164 AA773987 W96164 BM021522 |
| | | AI825903 AI824295 AA454622 BE833075 AW883986 BE833189 AI264049 AI090237 AI306153 |
| | | BF941075 BF369872 AW884073 |
| 425547 | 1228233_1 | AW963016 AA359181 AA359849 |
| 442257 | 18892_1 | AK055924 BC014104 BE889267 BI546677 BI549386 AL596984 AW503317 AW503831 |
| | | BE410616 BI600938 BI560459 BG980827 |
| 447128 | 11028_3 | BG292064 AI452509 AI271898 BE048502 BI966153 X84721 AI858001 BM021943 |
| | | AI553937 AI765259 F25787 AW015380 AA554539 AW059537 F35749 AA149853 AA961610 |
| | | AI568815 AW973696 C00201 R77127 BG438065 AI244810 H00719 |
| 407394 | 27110_2 | AF005081 BG193848 |
| 414213 | 5112_13 | BF311061 BG828926 BE262061 BI199425 T83915 AL554295 BG830004 BI335097 |
| | | BI193093 BF823498 BF823472 BM193195 AI952524 BF768990 BE781389 BI193044 BE297765 |
| 412595 | 35761_2 | AW964908 AA113749 BF907463 AA325520 BF911645 BF923879 BF928243 |
| 446924 | 626737_1 | AI694696 BE328826 BE504314 AI457297 AW134643 AI348387 |
| 441087 | 2642272_1 | AI797588 AI357156 AI204430 AA916498 |
| 409870 | 917085_1 | AW502321 AW502965 AW502314 AW502455 AW502282 AW502800 |
| 438870 | 52141_1 | AF075011 R63414 R63365 |
| 439285 | 22495_1 | AF086101 AL133916 AW955684 AW950828 AI346341 AI867454 BM263376 BF432231 |
| | | AI421279 AI655270 AW014882 BF439949 AA775552 N62351 AA626243 N59253 AI341407 |
| | | AA456968 AA457077 AI358918 AA364013 N79113 N54784 BE175639 N76721 AV727392 |
| | | Z45529 Z44343 F05908 F05403 F05398 |
| 417998 | 250632_1 | AW967420 AA210915 AA236991 AA210916 |

Pkey: Unique Eos probeset identifier number
CAT number: Gene cluster number
Accession: Genbank accession numbers

TABLE 8C

| Pkey | Ref | Strand | Nt_position |
|---|---|---|---|
| 405499 | 5762548 | Plus | 124845-125055, 131730-131833 |
| 406536 | 7711478 | Plus | 25655-25782 |
| 404608 | 9588566 | Minus | 22507-22637, 28093-28397 |
| 404502 | 7229863 | Minus | 56277-56819 |
| 403233 | 7637801 | Plus | 171338-171464, 172980-173099 |
| 402000 | 3935221 | Plus | 3935-4083, 7733-7845, 14982-15082 |
| 404672 | 9797204 | Minus | 24463-24607, 24711-24852, |
| | | | 25429-25565 |
| 406121 | 9143818 | Plus | 322375-323091 |
| 402356 | 8886972 | Minus | 90528-91265 |
| 406273 | 7543785 | Plus | 28641-29291 |
| 404033 | 8122195 | Plus | 7976-8156 |

Pkey: Unique number corresponding to an Eos probeset
Ref: Sequence source. The 7 digit numbers in this column are Genbank Identifier (Gl) numbers. “Dunham l. et al.” refers to the publication entitled “The DNA sequence of human chromosome 22.” Dunham l. et al., Nature (1999) 402: 489-495.
Strand: Indicates DNA strand from which exons were predicted.
Nt_position: Indicates nucleotide positions of predicted exons.

TABLE 9A
About 252 genes downregulated in lung metastases to the brain relative to primary lung tumors

| Pkey | ExAccn | UniGeneID | UniGene Title | R1 | SEQ ID NO(s): |
|---|---|---|---|---|---|
| 417204 | N81037 | Hs.1074 | surfactant, pulmonary-associated protein | 39.27 | 1071 |
| 418026 | BE379727 | Hs.83213 | fatty acid binding protein 4, adipocyte | 20.94 | 1148 |
| 424206 | NM_003734 | Hs.198241 | amine oxidase, copper containing 3 (vasc | 18.97 | 1815 5203 |
| 410677 | NM_003278 | Hs.65424 | tetranectin (plasminogen-binding protein | 7.71 | 4852 504 |
| 435684 | NM_001290 | Hs.4980 | LIM domain binding 2 | 11.20 | 2937 5568 |
| 415162 | AF035718 | Hs.78061 | transcription factor 21 | 6.62 | 4941 912 |
| 409385 | AA071267 | | gb:zm61g01.r1 Stratagene fibroblast (937 | 8.15 | 365 |
| 407694 | U77594 | Hs.37682 | retinoic acid receptor responder (tazaro | 11.84 | 181 4779 |
| 441835 | AB036432 | Hs.184 | advanced glycosylation end product-speci | 8.39 | 3375 5618 |
| 453655 | AW960427 | Hs.342874 | transforming growth factor, beta recepto | 4.25 | 4421 |
| 415274 | AF001548 | Hs.78344 | myosin, heavy polypeptide 11, smooth mus | 17.32 | 4944 921 |
| 412047 | AA934589 | Hs.49696 | ESTs | 6.27 | 605 |
| 423778 | Y09267 | Hs.132821 | flavin containing monooxygenase 2 | 7.36 | 1774 5187 |
| 407744 | AB020629 | Hs.38095 | ATP-binding cassette, sub-family A (ABC1 | 9.87 | 187 4781 |
| 429350 | AI754634 | Hs.131987 | ESTs | 5.41 | 2358 |
| 410016 | AA297977 | Hs.57907 | small inducible cytokine subfamily A (Cy | 10.30 | 429 |
| 407245 | X90568 | Hs.172004 | titin | 4.33 | 144 4768 |
| 426752 | X69490 | Hs.172004 | titin | 4.15 | 2093 5318 |
| 431723 | AW058350 | Hs.16762 | Homo sapiens mRNA; cDNA DKFZp564B2062 (f | 5.76 | 2590 |
| 428043 | T92248 | Hs.2240 | uteroglobin | 7.03 | 2216 |
| 435146 | R44557 | Hs.23748 | ESTs | 4.96 | 2899 |
| 416950 | AL049798 | Hs.80552 | dermatopontin | 11.09 | 1042 4972 |
| 441281 | BE501247 | Hs.144084 | ESTs | 6.42 | 3342 |
| 404246 | | | Target Exon | 6.16 | |
| 430998 | AF128847 | Hs.204038 | indolethylamine N-methyltransferase | 4.85 | 2524 5467 |
| 417355 | D13168 | Hs.82002 | endothelin receptor type B | 4.24 | 1085 4981 |
| 438150 | AA037534 | Hs.342874 | transforming growth factor, beta recepto | 4.17 | 3122 |
| 453676 | AW853745 | Hs.286035 | hypothetical protein FLJ22686 | 7.61 | 4423 |
| 422550 | BE297626 | Hs.296049 | microfibrillar-associated protein 4 | 5.37 | 1655 |
| 422667 | H25642 | | ESTs | 6.40 | 1670 |
| 423007 | AA320134 | Hs.196029 | Homo sapiens mRNA for KIAA1657 protein, | 4.69 | 1709 |
| 420225 | AW243046 | Hs.282076 | Homo sapiens mRNA for KIAA1650 protein, | 4.60 | 1397 |
| 424027 | AW337575 | Hs.201591 | ESTs | 4.34 | 1792 |
| 453180 | N46243 | Hs.110373 | ESTs, Highly similar to T42626 secreted | 5.15 | 4383 |
| 443062 | N77999 | Hs.8963 | Homo sapiens mRNA full length insert cDN | 4.61 | 3493 |
| 459247 | N46243 | Hs.110373 | ESTs, Highly similar to T42626 secreted | 4.55 | 4662 |
| 451154 | AA015879 | Hs.33536 | ESTs | 4.86 | 4198 |
| 438873 | AI302471 | Hs.124292 | Homo sapiens cDNA: FLJ23123 fis, clone L | 9.90 | 3173 |
| 456898 | NM_001928 | Hs.155597 | D component of complement (adipsin) | 3.65 | 4566 5803 |
| 418058 | AW161552 | Hs.83381 | guanine nucleotide binding protein 11 | 3.47 | 1154 |
| 410066 | AL117664 | Hs.58419 | DKFZP586L2024 protein | 4.26 | 438 4836 |
| 429640 | U83508 | Hs.2463 | angiopoietin 1 | 5.36 | 2400 5419 |
| 430468 | NM_004673 | Hs.241519 | angiopoietin-like 1 | 3.39 | 2489 5452 |
| 410023 | AB017169 | Hs.57929 | slit (Drosophila) homolog 3 | 3.84 | 431 4835 |
| 420105 | AW015571 | Hs.32244 | ESTs, Weakly similar to FMOD_HUMAN FIBRO | 6.62 | 1385 |
| 410545 | U32324 | Hs.64310 | interleukin 11 receptor, alpha | 4.99 | 4850 490 |
| 412002 | AA913024 | Hs.84698 | Homo sapiens cDNA FLJ14300 fis, clone PL | 4.47 | 601 |
| 444561 | NM_004469 | Hs.11392 | c-fos induced growth factor (vascular en | 3.51 | 3606 5641 |
| 425764 | AW996009 | Hs.112572 | Homo sapiens cDNA FLJ14130 fis, clone MA | 4.37 | 1994 |
| 408741 | M73720 | Hs.646 | carboxypeptidase A3 (mast cell) | 5.32 | 300 4805 |
| 418947 | W52990 | Hs.22860 | ESTs | 5.73 | 1261 |
| 419111 | AA234172 | Hs.137418 | ESTs | 6.96 | 1277 |
| 447371 | AA334274 | Hs.18368 | DKFZP564B0769 protein | 7.78 | 3851 |
| 416030 | H15261 | Hs.21948 | ESTs | 3.98 | 967 |
| 423690 | AA329648 | Hs.23804 | ESTs, Weakly similar to PN0099 son3 prot | 3.32 | 1767 |
| 412810 | M21574 | Hs.74615 | platelet-derived growth factor receptor, | 5.49 | 4888 672 |
| 413474 | T86312 | Hs.334485 | Homo sapiens cDNA FLJ14438 fis, clone HE | 7.27 | 725 |
| 439335 | AA742697 | Hs.62492 | NM_052863:Homo sapiens secretoglobin, fa | 5.16 | 3199 |
| 425498 | AL096725 | Hs.289010 | DKFZP434B103 protein | 5.76 | 1971 5267 |
| 445516 | R50291 | Hs.10846 | ESTs, Weakly similar to JH0783 diamine N | 8.70 | 3680 |
| 420626 | AF043722 | Hs.99491 | RAS guanyl releasing protein 2 (calcium | 4.39 | 1439 5083 |
| 416585 | X54162 | Hs.79386 | leiomodin 1, smooth muscle (LMOD1) (Thy | 3.40 | 1004 4964 |
| 427164 | AB037721 | Hs.173871 | KIAA1300 protein | 4.82 | 2129 5332 |
| 426488 | X03350 | Hs.4 | alcohol dehydrogenase 1B (class I), beta | 3.70 | 2071 5307 |
| 414290 | AI568801 | Hs.71721 | ESTs | 4.41 | 808 |
| 417967 | BE244373 | Hs.1119 | nuclear receptor subfamily 4, group A, m | 4.16 | 1142 |
| 451245 | AA016218 | Hs.58231 | ESTs | 4.45 | 4203 |
| 418807 | NM_004944 | Hs.88646 | deoxyribonuclease I-like 3 | 4.27 | 1242 5030 |
| 406923 | S70622 | | gb:G1 phase-specific gene {3′ region} [h | 3.49 | 110 4756 |
| 420556 | AA278300 | Hs.124292 | Homo sapiens cDNA: FLJ23123 fis, clone L | 8.19 | 1432 |
| 441499 | AW298235 | Hs.101689 | ESTs | 5.57 | 3354 |
| 443577 | AI078033 | Hs.177170 | ESTs, Weakly similar to ALU8_HUMAN ALU S | 4.94 | 3530 |
| 420174 | AI824144 | Hs.199749 | ESTs | 5.14 | 1391 |
| 454072 | AW572954 | Hs.252851 | ESTs | 3.49 | 4468 |
| 422195 | AB007903 | Hs.113082 | KIAA0443 gene product | 4.67 | 1614 5141 |
| 411988 | AA455459 | Hs.164480 | ESTs, Weakly similar to T50609 hypotheti | 5.45 | 599 |
| 414577 | AI056548 | Hs.72116 | hypothetical protein FLJ20992 similar to | 3.63 | 847 |
| 441723 | R72137 | Hs.7949 | DKFZP586B2420 protein | 4.64 | 3370 |
| 425887 | AL049443 | Hs.161283 | Homo sapiens mRNA; cDNA DKFZp586N2020 (f | 4.07 | 2010 |
| 447081 | Y13896 | Hs.17287 | potassium inwardly-rectifying channel, s | 3.81 | 3819 |
| 453355 | AW295374 | Hs.31412 | myopodin | 3.37 | 4400 |
| 458504 | AW070634 | Hs.144794 | ESTs | 4.36 | 4626 |
| 435823 | R07856 | Hs.16355 | ESTs | 3.28 | 2948 |
| 415386 | Z43087 | | gb:HSC13A121 normalized infant brain cDN | 3.32 | 929 |
| 448274 | AI268097 | Hs.67317 | Homo sapiens cDNA FLJ11775 fis, clone HE | 3.80 | 3954 |
| 425622 | AW360847 | Hs.16578 | ESTs | 3.66 | 1980 |
| 418965 | AI002238 | Hs.11482 | splicing factor, arginine/serine-rich 11 | 5.56 | 1264 |
| 450696 | AI654223 | Hs.16026 | hypothetical protein FLJ23191 | 3.86 | 4150 |
| 447742 | AF113925 | Hs.19405 | caspase recruitment domain 4 | 4.40 | 3899 5699 |
| 413802 | AW964490 | Hs.32241 | ESTs, Weakly similar to S65657 alpha-1C- | 3.61 | 754 |
| 431971 | BE274907 | Hs.77385 | myosin, light polypeptide 6, alkali, smo | 4.19 | 2622 |
| 455653 | BE154075 | | gb:PM0-HT0339-200400-010-E05 HT0339 Homo | 5.29 | 4518 |
| 406760 | AA829363 | | gb:of08g04.s1 NCI_CGAP_Co12 Homo sapiens | 3.56 | 98 |
| 443932 | AW888222 | Hs.9973 | tensin | 3.68 | 3563 |
| 421341 | AJ243212 | | deleted in malignant brain tumors 1 | 4.75 | 1509 5102 |
| 432485 | N90866 | Hs.276770 | CDW52 antigen (CAMPATH-1 antigen) | 3.75 | 2672 |
| 411962 | AA099050 | | gb:zk85d12.r1 Soares_pregnant_uterus_NbH | 4.43 | 594 |
| 453874 | AW591783 | Hs.36131 | collagen, type XIV, alpha 1 (undulin) | 5.05 | 4440 |
| 444769 | AI191650 | Hs.221436 | ESTs | 3.34 | 3626 |
| 451735 | AW407892 | | ESTs | 3.81 | 4244 |
| 446804 | AW572304 | Hs.271260 | ESTs, Moderately similar to dJ63G5.3 (H. | 3.39 | 3789 |
| 448515 | H68441 | Hs.13528 | hypothetical protein FLJ14054 | 3.47 | 3979 |
| 452816 | AA131789 | Hs.61509 | ESTs | 3.70 | 4346 |
| 439484 | AW970218 | Hs.135254 | Homo sapiens clone 1 thrombospondin mRNA | 4.04 | 3211 |
| 433688 | AA628467 | Hs.112572 | Homo sapiens cDNA FLJ14130 fis, clone MA | 4.03 | 2780 |
| 424580 | AA446539 | Hs.339024 | ESTs, Weakly similar to A46010 X-linked | 3.44 | 1860 |
| 447578 | AA912347 | Hs.136585 | ESTs, Weakly similar to JC5314 CDC28/cdc | 5.64 | 3882 |
| 447896 | AI436124 | Hs.294069 | Homo sapiens cDNA FLJ13384 fis, clone PL | 5.72 | 3920 |
| 417696 | BE241624 | Hs.82401 | CD69 antigen (p60, early T-cell activati | 4.90 | 1113 |
| 443679 | AK001810 | Hs.9670 | hypothetical protein FLJ10948 | 10.31 | 3538 5630 |
| 414516 | AI307802 | | ESTs, Weakly similar to T43458 hypotheti | 3.44 | 838 |
| 435916 | AW001885 | Hs.114103 | ESTs | 6.26 | 2952 |
| 452664 | AA398859 | Hs.18397 | hypothetical protein FLJ23221 | 4.22 | 4331 |
| 427890 | AA435761 | | ESTs | 6.61 | 2203 |
| 434666 | AF151103 | Hs.112259 | T cell receptor gamma locus | 3.69 | 2859 5551 |
| 450400 | AI694722 | Hs.279744 | ESTs | 3.33 | 4137 |
| 406800 | AA505535 | | gb:nh84h10.s1 NCI_CGAP_Br1.1 Homo sapien | 4.85 | 100 |
| 429655 | U48959 | Hs.211582 | myosin, light polypeptide kinase | 3.32 | 2402 5420 |
| 425701 | AA361850 | Hs.240443 | Human clone 137308 mRNA, partial cds | 4.16 | 1990 |
| 453767 | AB011792 | Hs.35094 | extracellular matrix protein 2, female o | 3.50 | 4430 5782 |
| 452336 | AA960961 | Hs.305953 | zinc finger protein 83 (HPF1) | 5.80 | 4298 |
| 418117 | AI922013 | Hs.83496 | linker for activation of T cells | 3.91 | 1162 |
| 407753 | AL045916 | Hs.293419 | ESTs | 4.04 | 189 |
| 428780 | AI478578 | Hs.153714 | ESTs | 3.53 | 2296 |
| 429439 | AL049268 | Hs.202684 | Homo sapiens mRNA; cDNA DKFZp564G103 (fr | 3.85 | 2370 |
| 412000 | AW576555 | Hs.15780 | ATP-binding cassette, sub-family A (ABC1 | 3.47 | 600 |
| 448051 | BE615453 | Hs.346509 | dedicator of cyto-kinesis 1 | 3.56 | 3938 |
| 406801 | AW242054 | Hs.190813 | ribosomal protein L9 | 4.37 | 101 |
| 424698 | AA164366 | Hs.151973 | hypothetical protein FLJ23511 | 4.84 | 1875 |
| 442176 | AA983764 | Hs.128910 | ESTs | 4.17 | 3403 |
| 416427 | BE244050 | Hs.79307 | Rac/Cdc42 guanine exchange factor (GEF) | 4.54 | 997 |
| 413156 | AA127133 | | gb:zI87e03.r1 Stratagene colon (937204) | 3.65 | 696 |
| 424078 | AB006625 | Hs.139033 | paternally expressed 3 | 4.00 | 1800 5196 |
| 417410 | AF063020 | Hs.82110 | PC4 and SFRS1 interacting protein 1 | 4.01 | 1093 4984 |
| 434958 | T99949 | Hs.303428 | Homo sapiens cDNA FLJ14832 fis, clone OV | 3.58 | 2883 |
| 427544 | AI767152 | Hs.181400 | ESTs, Weakly similar to I78885 serine/th | 3.47 | 2163 |
| 453510 | AI699482 | Hs.42151 | ESTs | 4.15 | 4415 |
| 442287 | AW952703 | Hs.8182 | synaptic nuclei expressed gene 1b | 3.46 | 3411 |
| 414142 | AW368397 | Hs.334485 | hemicentin (fibulin 6) | 4.90 | 792 |
| 421512 | AB007923 | Hs.265848 | myomegalin | 3.41 | 1531 5112 |
| 437176 | AW176909 | Hs.42346 | calcineurin-binding protein calsarcin-1 | 5.31 | 3042 |
| 426992 | BE244961 | Hs.343200 | FE65-LIKE 2 | 4.74 | 2118 |
| 431615 | AW295859 | Hs.235860 | ESTs | 3.76 | 2576 |
| 458368 | BE504731 | Hs.138827 | ESTs | 3.77 | 4620 |
| 406282 | | | Homo sapiens mRNA full length insert cDN | 3.62 | |
| 411546 | BE172648 | Hs.75415 | beta-2-microglobulin | 4.35 | 559 |
| 421956 | AA301180 | | gb:EST14093 Testis tumor Homo sapiens cD | 5.12 | 1585 |
| 422964 | AW439476 | Hs.256895 | ESTs | 4.15 | 1702 |
| 410544 | AI446543 | Hs.95511 | ESTs | 3.33 | 489 |
| 459508 | R83265 | Hs.205956 | EST | 3.39 | 4670 |
| 409038 | T97490 | Hs.50002 | small inducible cytokine subfamily A (Cy | 4.81 | 326 |
| 421823 | N40850 | Hs.28625 | ESTs | 6.96 | 1568 |
| 407568 | AA740964 | Hs.62699 | ESTs | 3.32 | 168 |
| 430810 | AI742514 | Hs.158732 | ESTs | 3.34 | 2515 |
| 423705 | F11425 | | ribosomal protein S3 | 5.50 | 1769 |
| 436553 | AW407157 | Hs.8997 | immunoglobulin lambda locus | 4.97 | 2992 |
| 403325 | | | C2000428*:gi\|7705383\|ref\|NP_057536.1\| GC | 4.59 | |
| 437255 | R58970 | Hs.9887 | ESTs | 3.58 | 3049 |
| 439665 | W93979 | | gb:zd97h10.r1 Soares_fetal_heart_NbHH19W | 3.54 | 3226 |
| 450000 | AI952797 | Hs.10888 | hypothetical protein FLJ21709 | 3.39 | 4096 |
| 449787 | AA005341 | | ESTs | 3.69 | 4082 |
| 429760 | AW137682 | Hs.134589 | ESTs | 3.39 | 2414 |
| 442070 | BE244622 | Hs.8084 | hypothetical protein dJ465N24.2.1 | 3.71 | 3394 |
| 415733 | AI052628 | Hs.271570 | ESTs, Weakly similar to 2109260A B cell | 3.35 | 947 |
| 424084 | AI940675 | Hs.20914 | hypothetical protein FLJ23056 | 3.51 | 1802 |
| 427732 | NM_002980 | Hs.2199 | secretin receptor | 3.66 | 2191 5345 |
| 431177 | NM_003304 | Hs.250687 | transient receptor potential channel 1 | 3.67 | 2536 5471 |
| 447571 | AF274863 | Hs.18889 | DKFZP434M183 protein | 3.55 | 3880 5693 |
| 458018 | AI199575 | Hs.37716 | ESTs | 4.63 | 4606 |
| 453880 | AI803166 | Hs.135121 | ESTs, Weakly similar to I38022 hypotheti | 4.21 | 4443 |
| 425456 | T70445 | Hs.157850 | ribosomal protein L9 | 5.19 | 1967 |
| 442587 | AK001846 | Hs.8412 | Homo sapiens cDNA FLJ10984 fis, clone PL | 3.53 | 3449 |
| 443998 | AI620661 | Hs.296276 | ESTs | 4.77 | 3567 |
| 435289 | AA677540 | Hs.117064 | ESTs | 3.50 | 2907 |
| 445837 | AI261700 | Hs.145544 | ESTs | 4.98 | 3702 |
| 442561 | NM_013450 | Hs.8383 | bromodomain adjacent to zinc finger doma | 6.60 | 3442 5621 |
| 433205 | AB040901 | Hs.23542 | KIAA1468 protein | 3.40 | 2748 5533 |
| 422241 | Y00062 | Hs.170121 | protein tyrosine phosphatase, receptor t | 3.32 | 1617 5142 |
| 437365 | AW965771 | Hs.91065 | hypothetical protein DKFZp761B2423 | 3.94 | 3061 |
| 419902 | AA804409 | Hs.118920 | ESTs | 3.47 | 1367 |
| 445618 | H79667 | Hs.237642 | Homo sapiens cDNA FLJ12052 fis, clone HE | 3.86 | 3688 |
| 433019 | AI208513 | Hs.279915 | translocase of inner mitochondrial membr | 4.10 | 2723 |
| 425231 | AA527161 | | ESTs | 3.56 | 1939 |
| 428046 | AW812795 | Hs.337534 | ESTs, Moderately similar to I38022 hypot | 5.99 | 2217 |
| 409667 | AW452447 | Hs.283107 | ESTs, Weakly similar to T22143 hypotheti | 3.32 | 392 |
| 414474 | AA147968 | Hs.31297 | duodenal cytochrome b | 3.65 | 834 |
| 422128 | AW881145 | | gb:QV0-OT0033-010400-182-a07 OT0033 Homo | 6.35 | 1607 |
| 447754 | AW073310 | Hs.163533 | intron of HER4 | 3.47 | 3900 |
| 422276 | AL137690 | Hs.114173 | Homo sapiens mRNA; cDNA DKFZp434O032 (fr | 3.48 | 1625 5145 |
| 451678 | AA374181 | Hs.26799 | DKFZP564D0764 protein | 4.51 | 4238 |
| 451744 | AI813757 | | F-box only protein 29 | 3.58 | 4246 |
| 449609 | BE246434 | Hs.289026 | guanine nucleotide binding protein (G pr | 3.46 | 4070 |
| 426276 | AW881411 | Hs.169078 | hypothetical protein FLJ23018 | 3.51 | 2047 |
| 440602 | AI743491 | Hs.292692 | ESTs | 3.44 | 3303 |
| 433253 | AW450502 | Hs.24218 | ESTs | 3.37 | 2750 |
| 406757 | T65957 | Hs.77039 | ATP synthase, H transporting, mitochondr | 3.73 | 97 |
| 417032 | AA192469 | Hs.271838 | ESTs | 3.56 | 1054 |
| 438086 | AA336519 | Hs.83623 | nuclear receptor subfamily 1, group I, m | 3.30 | 3116 |
| 436987 | AA740990 | Hs.120551 | toll-like receptor 10 | 4.31 | 3025 |
| 407374 | AA724738 | Hs.131034 | ESTs, Weakly similar to I78885 serine/th | 5.21 | 157 |
| 412088 | AI689496 | Hs.108932 | ESTs | 4.12 | 606 |
| 447022 | AW291223 | Hs.157573 | ESTs | 4.48 | 3812 |
| 450654 | AJ245587 | Hs.25275 | Kruppel-type zinc finger protein | 3.51 | 4144 5733 |
| 450701 | H39960 | Hs.288467 | hypothetical protein XP_098151 (leucine- | 5.43 | 4152 |
| 408491 | AI088063 | Hs.7882 | ESTs | 5.35 | 272 |
| 451323 | AI903313 | Hs.34579 | ESTs, Moderately similar to ALU6_HUMAN A | 3.30 | 4211 |
| 407183 | AA358015 | | gb:EST66864 Fetal lung III Homo sapiens | 3.50 | 134 |
| 436749 | AA584890 | Hs.5302 | lectin, galactoside-binding, soluble, 4 | 9.22 | 3006 |
| 439138 | AI742605 | Hs.193696 | ESTs | 3.33 | 3188 |
| 450219 | AI826999 | Hs.224624 | ESTs | 3.32 | 4114 |
| 456373 | BE247706 | Hs.89751 | membrane-spanning 4-domains, subfamily A | 4.20 | 4539 |
| 424677 | U09414 | | zinc finger protein 137 (clone pHZ-30) | 3.55 | 1871 5225 |
| 451240 | AJ131693 | Hs.58103 | A kinase (PRKA) anchor protein (yotiao) | 3.46 | 4202 5739 |
| 437205 | AL110232 | Hs.279243 | Homo sapiens mRNA; cDNA DKFZp564D2071 (f | 3.36 | 3044 |
| 421893 | NM_001078 | Hs.109225 | vascular cell adhesion molecule 1 | 3.93 | 1577 5127 |
| 429493 | AL134708 | Hs.145998 | ESTs | 3.98 | 2379 |
| 449539 | W80363 | Hs.58446 | ESTs | 3.55 | 4065 |
| 417339 | AI912592 | Hs.7882 | ESTs | 4.62 | 1082 |
| 448878 | AW204582 | Hs.224906 | ESTs | 3.35 | 4017 |
| 425247 | NM_005940 | Hs.155324 | matrix metalloproteinase 11 (stromelysin | 5.62 | 1943 5253 |
| 418310 | AA814100 | Hs.86693 | ESTs | 3.86 | 1180 |
| 403903 | | | C5001632*: gi\|10645308\|gb\|AAG21430.1\|AC00 | 4.23 | |
| 437644 | AA748575 | Hs.136748 | lectin-like NK cell receptor | 3.66 | 3077 |
| 450788 | AI738410 | | ESTs | 3.94 | 4165 |
| 453392 | U23752 | Hs.32964 | SRY (sex determining region Y)-box 11 | 4.87 | 4403 5776 |
| 407758 | D50915 | Hs.38365 | KIAA0125 gene product | 4.09 | 192 4782 |
| 419556 | U29615 | Hs.91093 | chitinase 1 (chitotriosidase) | 4.46 | 1332 5051 |
| 439854 | T62993 | Hs.347609 | gb:yb99a06.r1 Stratagene lung (937210) H | 3.30 | 3239 |
| 430770 | AA765694 | Hs.123296 | ESTs | 3.94 | 2512 |
| 420224 | M84371 | Hs.96023 | CD19 antigen | 4.13 | 1396 5068 |
| 419255 | AA235672 | Hs.87491 | ESTs | 3.89 | 1294 |
| 422583 | AA410506 | Hs.27973 | KIAA0874 protein | 3.69 | 1660 |
| 445669 | AI570830 | Hs.174870 | ESTs | 3.64 | 3693 |
| 447932 | AA837474 | Hs.20021 | vesicle-associated membrane protein 1 (s | 3.37 | 3922 |
| 411982 | H53601 | | gb:yq87g05.s1 Soares fetal liver spleen | 3.52 | 598 |
| 451583 | AI653797 | Hs.24133 | ESTs | 3.32 | 4230 |
| 443184 | AI638728 | Hs.131973 | ESTs | 3.73 | 3505 |
| 440650 | R44692 | Hs.326801 | Human DNA sequence from PAC 75N13 on chr | 4.06 | 3309 |
| 418007 | M13509 | Hs.83169 | matrix metalloproteinase 1 (interstitial | 6.85 | 1146 4998 |
| 452107 | AB020681 | Hs.27973 | KIAA0874 protein | 3.35 | 4271 5755 |
| 407811 | AW190902 | Hs.40098 | cysteine knot superfamily 1, BMP antagon | 3.68 | 199 |
| 455002 | AW850286 | | gb:IL3-CT0219-161199-031-H11 CT0219 Homo | 3.53 | 4499 |
| 408063 | BE086548 | Hs.42346 | calcineurin-binding protein calsarcin-1 | 3.88 | 230 |
| 417689 | AA828347 | Hs.90998 | KIAA0128 protein; septin 2 | 3.48 | 1111 |
| 421361 | AI246129 | | ESTs | 3.63 | 1512 |
| 458080 | BE142728 | | gb:MR0-HT0157-021299-004-d08 HT0157 Homo | 3.33 | 4608 |
| 422867 | L32137 | Hs.1584 | cartilage oligomeric matrix protein (pse | 4.41 | 1687 5160 |
| 414085 | AA114016 | Hs.75746 | aldehyde dehydrogenase 1 family, member | 3.39 | 786 |
| 456034 | AW450979 | | gb:UI-H-BI3-ala-a-12-0-UI.s1 NCI_CGAP_Su | 3.44 | 4527 |
| 427585 | D31152 | Hs.179729 | collagen, type X, alpha 1 (Schmid metaph | 4.10 | 2169 |
| 406964 | M21305 | | FGENES predicted novel secreted protein | 3.98 | 114 4760 |
| 411089 | AA456454 | | cell division cycle 2-like 1 (PITSLRE pr | 3.64 | 534 |

Pkey: Unique Eos probeset identifier number
ExAccn: Exemplar Accession number, Genbank accession number
UniGeneID: UniGene number
UniGene Title: UniGene gene title
R1: 90th percentile of lung adenocarcinoma Als divided by the 90th percentile of lung metastases to the brain Als, where the 15th percentile of all normal body tissue Als was subtracted from the numerator and denominator.
SEQ ID NO(s): SEQ ID number(s) for nucleic acid and protein sequences associated with table entry.

TABLE 9B

| Pkey | CAT number | Accession |
|---|---|---|
| 409385 | 110758_1 | T65940 T64515 AA071267 AA071334 |
| 422667 | 224778_1 | AI758223 AW469334 BF940841 AW080348 AI270363 |
| | | AI055892 BE464168 BF431797 BE350144 BF448739 AI693409 BF432999 D62848 |
| | | AA398070 AI383375 AW611490 |
| 415386 | 11281_5 | R95033 H15506 Z43087 F07410 H54108 H98000 |
| 455653 | 200977_1 | BF820280 BE154075 BE154018 BE153509 BE153973 BE064861 |
| | | BE065075 BE064684 BE153602 BE153852 BE153847 BE064772 BE064842 BE153557 |
| 406760 | 0_0 | AA829363 |
| 421341 | 1407_1 | NM_007329 AF159456 AJ243212 AJ297935 AA295769 |
| | | NM_017579 AJ243224 AI492875 AI796676 AI749838 AA918144 AI814590 |
| | | AI923531 BF513992 AI720725 AI150879 AI279072 AW612904 AI492104 AI284510 AI141231 |
| | | AA613554 AW662148 AW769047 AA565985 AW612888 AU100513 BG955585 |
| | | BG955588 AA295763 BE829414 BF760645 BG954398 AA295332 AA295795 |
| | | BE932867 AW769569 T89953 BE934311 |
| 411962 | 2307710_1 | AA099050 AA099526 T47733 |
| 451735 | 480028_1 | BG113814 AW407892 AI812096 AW076043 |
| 414516 | 60847_1 | AK057782 AI146454 BG703115 AI765980 AI948611 AA889263 AA947457 |
| | | BG547193 H11947 Z38147 F10426 BF447329 H11946 T74968 F06195 BG548563 |
| | | AI004988 AA148735 AI307802 AI439791 BE041453 AI984904 AA148734 F12823 |
| 427890 | 1373988_1 | AA417099 AA435761 AA972917 AI660387 |
| 406800 | 0_0 | AA505535 |
| 413156 | 1224380_1 | AW958912 AA384396 T72119 AA127133 |
| 421956 | 461103_1 | AV750327 AA300961 AA301180 AW963779 |
| 423705 | 2901_38 | BG875506 AI299893 AA989327 BE708123 AI859076 AI434010 AA347691 |
| | | F11425 AA329878 |
| 439665 | 24497_1 | AF086498 AW955697 W93979 AI936062 AW268568 AA364461 W93980 |
| | | BI914847 BI523483 |
| 449787 | 79759_1 | AW975633 AW961632 AA322539 AW513289 AI864190 AW073506 AI016894 |
| | | AA747635 AA730405 AA418033 AI609549 AI873981 AA005341 BE182658 |
| | | AA004291 |
| 425231 | 235504_1 | AA527161 BG211784 AA527065 AA505489 AW512550 |
| 422128 | 17516_16 | BI261864 BI036453 BI023096 BI023388 AA331991 AV721898 AW881145 |
| | | AA490718 M85637 T06067 AV761102 BI023091 BI022906 |
| 451744 | 18159_4 | BG165570 AI973190 BE221960 AI813757 |
| 424677 | 2518_37 | U09414 NM_003438 AA503545 AI022449 AA043458 AA766074 |
| | | AA765442 AA805052 AI028211 AW609708 |
| 450788 | 513262_1 | AI971725 AI738410 AW016905 |
| 411982 | 1144977_1 | AW880868 BE003925 H53601 |
| 455002 | 1089092_1 | BE143505 BF374194 BF374190 AW850286 |
| 421361 | 267843_1 | AA287780 AI246129 AI708426 Z36873 |
| 458080 | 1164245_1 | BI602455 AW937124 AA834047 BE142728 |
| 456034 | 685586_1 | AA136653 AA136656 AW450979 AA984358 AA809054 AW238038 |
| | | AA492073 BE168945 |
| 411089 | 5597_6 | BI009308 BI009893 BF922023 BF922909 BF922913 BF922096 BF957733 |
| | | BE701791 AA456454 AA579876 BF933710 AA091294 BI00291 AW905577 |
| | | AW975593 AA713730 AW836781 AA666384 AA551106 BF594606 AI082382 |
| | | AI955808 AI679895 AI679386 BF435555 AA586369 AA551351 AA595822 |
| | | AA565188 BF808855 AA584921 N86077 AA601031 AA633188 AA514764 AA454562 |
| | | AA551297 AA936109 BI009389 AW897806 BE815442 BF739374 BI009310 |
| | | BF925422 BF933709 BF922034 BF925465 BI009680 |

Pkey: Unique Eos probeset identifier number
CAT number: Gene cluster number
Accession: Genbank accession numbers

TABLE 9C

| Pkey | Ref | Strand | Nt_position |
|---|---|---|---|
| 404246 | 7406725 | Plus | 82477-82628, 82721-82817, |
| | | | 82910-83071, 8314 |
| 406282 | 7549620 | Plus | 61692-61955 |
| 403325 | 8440025 | Minus | 109763-109926 |
| 403903 | 7710671 | Minus | 101165-102597 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 10A
About 289 genes downregulated in lung metastases to the brain relative to normal lung

| | |
|---|---|
| Pkey: | Unique Eos probeset identifier number |
| ExAccn: | Exemplar Accession number, Genbank accession number |
| UniGeneID: | UniGene number |
| UniGene Title: | UniGene gene title |
| R1: | 90th percentile of normal lung Als divided by the 90th percentile of lung metastases to the |
| | brain Als, where the 15th percentile of all normal body tissue Als was subtracted from the |
| | numerator and denominator. |
| SEQ ID NO(s): | SEQ ID number(s) for nucleic acid and protein sequences associated with table entry. |

| Pkey | ExAccn | UniGeneID | UniGene Title | R1 | SEQ ID NO(s): |
|---|---|---|---|---|---|
| 417204 | N81037 | Hs.1074 | surfactant, pulmonary-associated protein | 50.05 | 1071 |
| 418026 | BE379727 | Hs.83213 | fatty acid binding protein 4, adipocyte | 31.39 | 1148 |
| 424206 | NM_003734 | Hs.198241 | amine oxidase, copper containing 3 (vasc | 29.42 | 1815 5203 |
| 410677 | NM_003278 | Hs.65424 | tetranectin (plasminogen-binding protein | 24.89 | 4852 504 |
| 435684 | NM_001290 | Hs.4980 | LIM domain binding 2 | 18.00 | 2937 5568 |
| 415162 | AF035718 | Hs.78061 | transcription factor 21 | 15.10 | 4941 912 |
| 409385 | AA071267 | | gb: zm61g01.r1 Stratagene fibroblast (937 | 14.40 | 365 |
| 407694 | U77594 | Hs.37682 | retinoic acid receptor responder (tazaro | 14.18 | 181 4779 |
| 441835 | AB036432 | Hs.184 | advanced glycosylation end product-speci | 14.15 | 3375 5618 |
| 453655 | AW960427 | Hs.342874 | transforming growth factor, beta recepto | 13.42 | 4421 |
| 415274 | AF001548 | Hs.78344 | myosin, heavy polypeptide 11, smooth mus | 12.95 | 4944 921 |
| 412047 | AA934589 | Hs.49696 | ESTs | 11.68 | 605 |
| 423778 | Y09267 | Hs.132821 | flavin containing monooxygenase 2 | 10.61 | 1774 5187 |
| 407744 | AB020629 | Hs.38095 | ATP-binding cassette, sub-family A (ABC1 | 10.36 | 187 4781 |
| 427019 | AA001732 | Hs.173233 | hypothetical protein FLJ10970 | 9.80 | 2122 |
| 429350 | AI754634 | Hs.131987 | ESTs | 9.51 | 2358 |
| 410016 | AA297977 | Hs.57907 | small inducible cytokine subfamily A (Cy | 9.49 | 429 |
| 453310 | X70697 | Hs.553 | solute carrier family 6 (neurotransmitte | 9.47 | 4394 5774 |
| 407245 | X90568 | Hs.172004 | titin | 9.22 | 144 4768 |
| 426752 | X69490 | Hs.172004 | titin | 9.13 | 2093 5318 |
| 431723 | AW058350 | Hs.16762 | Homo sapiens mRNA; cDNA DKFZp564B2062 (f | 9.06 | 2590 |
| 428043 | T92248 | Hs.2240 | uteroglobin | 8.96 | 2216 |
| 435146 | R44557 | Hs.23748 | ESTs | 8.56 | 2899 |
| 416950 | AL049798 | Hs.80552 | dermatopontin | 8.53 | 1042 4972 |
| 441281 | BE501247 | Hs.144084 | ESTs | 8.52 | 3342 |
| 404246 | | | Target Exon | 8.39 | |
| 432128 | AA127221 | Hs.296502 | Interleukin 1 receptor-like 1 | 8.35 | 2633 |
| 430998 | AF128847 | Hs.204038 | indolethylamine N-methyltransferase | 8.20 | 2524 5467 |
| 417355 | D13168 | Hs.82002 | endothelin receptor type B | 8.18 | 1085 4981 |
| 422287 | F16365 | Hs.114346 | cytochrome c oxidase subunit VIIa polype | 8.16 | 1628 |
| 438150 | AA037534 | Hs.342874 | transforming growth factor, beta recepto | 8.09 | 3122 |
| 453676 | AW853745 | Hs.286035 | hypothetical protein FLJ22686 | 8.00 | 4423 |
| 422550 | BE297626 | Hs.296049 | microfibrillar-associated protein 4 | 7.73 | 1655 |
| 422667 | H25642 | | ESTs | 7.50 | 1670 |
| 423007 | AA320134 | Hs.196029 | Homo sapiens mRNA for KIAA1657 protein, | 7.36 | 1709 |
| 454229 | AW957744 | Hs.278469 | lacrimal proline rich protein | 7.15 | 4473 |
| 436394 | AA531187 | Hs.126705 | ESTs | 7.10 | 2982 |
| 453574 | AI767947 | Hs.50841 | ESTs | 7.07 | 4418 |
| 420225 | AW243046 | Hs.282076 | Homo sapiens mRNA for KIAA1650 protein, | 6.88 | 1397 |
| 443709 | AI082692 | Hs.134662 | ESTs | 6.79 | 3542 |
| 424027 | AW337575 | Hs.201591 | ESTs | 6.78 | 1792 |
| 428769 | AW207175 | Hs.106771 | ESTs | 6.69 | 2293 |
| 414464 | AI870175 | Hs.13957 | ESTs | 6.67 | 833 |
| 453180 | N46243 | Hs.110373 | ESTs, Highly similar to T42626 secreted | 6.51 | 4383 |
| 443062 | N77999 | Hs.8963 | Homo sapiens mRNA full length insert cDN | 6.48 | 3493 |
| 459247 | N46243 | Hs.110373 | ESTs, Highly similar to T42626 secreted | 6.29 | 4662 |
| 451154 | AA015879 | Hs.33536 | ESTs | 6.13 | 4198 |
| 438873 | AI302471 | Hs.124292 | Homo sapiens cDNA: FLJ23123 fis, clone L | 6.08 | 3173 |
| 411939 | AI365585 | Hs.146246 | ESTs | 5.97 | 587 |
| 456898 | NM_001928 | Hs.155597 | D component of complement (adipsin) | 5.97 | 4566 5803 |
| 418058 | AW161552 | Hs.83381 | guanine nucleotide binding protein 11 | 5.90 | 1154 |
| 410066 | AL117664 | Hs.58419 | DKFZP586L2024 protein | 5.64 | 438 4836 |
| 439745 | AL389981 | | Homo sapiens mRNA full length insert cDN | 5.60 | 3232 |
| 401974 | | | NM_018896*: Homo sapiens calcium channel, | 5.56 | 31 4701 |
| 429640 | U83508 | Hs.2463 | angiopoietin 1 | 5.55 | 2400 5419 |
| 421218 | NM_000499 | Hs.72912 | cytochrome P450, subfamily I (aromatic c | 5.51 | 1490 5098 |
| 430310 | U60115 | Hs.239069 | four and a half LIM domains 1 | 5.50 | 2468 5443 |
| 430468 | NM_004673 | Hs.241519 | angiopoietin-like 1 | 5.47 | 2489 5452 |
| 413948 | C05145 | Hs.75636 | myosin light chain 2a | 5.37 | 768 |
| 410023 | AB017169 | Hs.57929 | slit (Drosophila) homolog 3 | 5.20 | 431 4835 |
| 418935 | T28499 | Hs.89485 | carbonic anhydrase IV | 5.20 | 1259 |
| 446017 | N98238 | Hs.55185 | ESTs | 5.15 | 3718 |
| 420105 | AW015571 | Hs.32244 | ESTs, Weakly similar to FMOD_HUMAN FIBRO | 5.11 | 1385 |
| 420958 | AA309431 | Hs.66 | interleukin 1 receptor-like 1 | 5.10 | 1469 |
| 409153 | W03754 | Hs.50813 | hypothetical protein FLJ20022 | 5.07 | 342 |
| 410545 | U32324 | Hs.64310 | interleukin 11 receptor, alpha | 5.07 | 4850 490 |
| 412002 | AA913024 | Hs.84698 | Homo sapiens cDNA FLJ14300 fis, clone PL | 5.07 | 601 |
| 442832 | AW206560 | Hs.253569 | ESTs | 5.06 | 3474 |
| 444561 | NM_004469 | Hs.11392 | c-fos induced growth factor (vascular en | 5.06 | 3606 5641 |
| 431848 | AI378857 | Hs.271605 | ESTs, Highly similar to AF175283 1 zinc | 4.97 | 2606 |
| 425764 | AW996009 | Hs.112572 | Homo sapiens cDNA FLJ14130 fis, clone MA | 4.94 | 1994 |
| 427605 | NM_000997 | Hs.337445 | ribosomal protein L37 | 4.93 | 2171 5340 |
| 450008 | H52970 | Hs.36688 | WAP four-disulfide core domain 1 | 4.90 | 4097 |
| 421177 | AW070211 | Hs.102415 | Homo sapiens mRNA; cDNA DKFZp586N0121 (f | 4.86 | 1485 |
| 451607 | AI439103 | Hs.7945 | AIE-75 binding protein protein | 4.86 | 4232 |
| 408741 | M73720 | Hs.646 | carboxypeptidase A3 (mast cell) | 4.85 | 300 4805 |
| 418947 | W52990 | Hs.22860 | ESTs | 4.85 | 1261 |
| 419111 | AA234172 | Hs.137418 | ESTs | 4.82 | 1277 |
| 447371 | AA334274 | Hs.18368 | DKFZP564B0769 protein | 4.77 | 3851 |
| 416030 | H15261 | Hs.21948 | ESTs | 4.77 | 967 |
| 423690 | AA329648 | Hs.23804 | ESTs, Weakly similar to PN0099 son3 prot | 4.74 | 1767 |
| 439551 | W72062 | Hs.11112 | ESTs | 4.73 | 3214 |
| 412810 | M21574 | Hs.74615 | platelet-derived growth factor receptor, | 4.71 | 4888 672 |
| 424310 | AA338648 | Hs.50334 | testes development-related NYD-SP22 | 4.70 | 1828 |
| 413474 | T86312 | Hs.334485 | Homo sapiens cDNA FLJ14438 fis, clone HE | 4.66 | 725 |
| 423099 | NM_002837 | Hs.123641 | protein tyrosine phosphatase, receptor t | 4.63 | 1718 5168 |
| 439335 | AA742697 | Hs.62492 | NM_052863: Homo sapiens secretoglobin, fa | 4.60 | 3199 |
| 425498 | AL096725 | Hs.289010 | DKFZP434B103 protein | 4.59 | 1971 5267 |
| 445516 | R50291 | Hs.10846 | ESTs, Weakly similar to JH0783 diamine N | 4.58 | 3680 |
| 420626 | AF043722 | Hs.99491 | RAS guanyl releasing protein 2 (calcium | 4.58 | 1439 5083 |
| 408374 | AW025430 | Hs.155591 | forkhead box F1 | 4.57 | 261 |
| 440647 | BE397806 | | poly(A)-binding protein, cytoplasmic 1 | 4.56 | 3308 |
| 416986 | AI471952 | Hs.49776 | ESTs | 4.55 | 1048 |
| 416585 | X54162 | Hs.79386 | leiomodin 1, smooth muscle (LMOD1) (Thy | 4.53 | 1004 4964 |
| 407891 | AA486620 | Hs.41135 | endomucin-2 | 4.50 | 212 |
| 409064 | AA062954 | Hs.141883 | ESTs | 4.49 | 330 |
| 427164 | AB037721 | Hs.173871 | KIAA1300 protein | 4.39 | 2129 5332 |
| 426488 | X03350 | Hs.4 | alcohol dehydrogenase 1B (class I), beta | 4.36 | 2071 5307 |
| 410036 | R57171 | Hs.57975 | calsequestrin 2 (cardiac muscle) | 4.32 | 434 |
| 447205 | BE617015 | Hs.11006 | ESTs, Moderately similar to T17372 plasm | 4.29 | 3829 |
| 418307 | U70867 | Hs.83974 | solute carrier family 21 (prostaglandin | 4.27 | 1179 5008 |
| 441003 | BE172240 | Hs.126379 | ESTs, Weakly similar to I38022 hypotheti | 4.22 | 3324 |
| 414290 | AI568801 | Hs.71721 | ESTs | 4.22 | 808 |
| 423387 | AJ012074 | | vasoactive intestinal peptide receptor 1 | 4.19 | 1740 5178 |
| 413956 | AI821351 | Hs.193133 | ESTs, Weakly similar to ALU7_HUMAN ALU S | 4.16 | 770 |
| 417967 | BE244373 | Hs.1119 | nuclear receptor subfamily 4, group A, m | 4.15 | 1142 |
| 424711 | NM_005795 | Hs.152175 | calcitonin receptor-like | 4.12 | 1876 5227 |
| 427983 | M17706 | Hs.2233 | colony stimulating factor 3 (granulocyte | 4.12 | 2211 |
| 456676 | AI870001 | Hs.334479 | ESTs, Moderately similar to KIAA1139 pro | 4.10 | 4556 |
| 451245 | AA016218 | Hs.58231 | ESTs | 4.10 | 4203 |
| 424433 | H04607 | Hs.78061 | ESTs | 4.09 | 1844 |
| 452541 | AA022569 | Hs.29802 | slit (Drosophila) homolog 2 | 4.09 | 4324 |
| 424137 | AA335769 | Hs.16262 | ESTs | 4.09 | 1808 |
| 423334 | AK000906 | Hs.127273 | hypothetical protein FLJ10044 | 4.08 | 1737 5176 |
| 422640 | M37984 | Hs.118845 | troponin C, slow | 4.06 | 1667 5155 |
| 418807 | NM_004944 | Hs.88646 | deoxyribonuclease I-like 3 | 4.04 | 1242 5030 |
| 406923 | S70622 | | gb: G1 phase-specific gene {3′ region} [h | 4.04 | 110 4756 |
| 420556 | AA278300 | Hs.124292 | Homo sapiens cDNA: FLJ23123 fis, clone L | 4.03 | 1432 |
| 424054 | AA334511 | Hs.26638 | membrane-spanning 4-domains, subfamily A | 4.02 | 1796 |
| 441499 | AW298235 | Hs.101689 | ESTs | 4.02 | 3354 |
| 420256 | U84722 | Hs.76206 | cadherin 5, type 2, VE-cadherin (vascula | 4.02 | 1401 5071 |
| 427383 | NM_005411 | Hs.177582 | surfactant, pulmonary-associated protein | 4.01 | 2150 5337 |
| 433855 | AA834082 | Hs.307559 | ESTs | 3.99 | 2792 |
| 403421 | | | NM_016369*: Homo sapiens claudin 18 (CLDN | 3.98 | 45 4714 |
| 401929 | | | C17001690: gi\|6005701\|ref\|NP_009099.1\|AT | 3.98 | |
| 443577 | AI078033 | Hs.177170 | ESTs, Weakly similar to ALU8_HUMAN ALU S | 3.98 | 3530 |
| 445624 | AW140103 | Hs.78880 | ilvB (bacterial acetolactate synthase)-I | 3.97 | 3689 |
| 408614 | AL137698 | Hs.46531 | Homo sapiens mRNA; cDNA DKFZp434C1915 (f | 3.96 | 283 4802 |
| 420174 | AI824144 | Hs.199749 | ESTs | 3.96 | 1391 |
| 434975 | AA657884 | Hs.314413 | ESTs | 3.95 | 2885 |
| 418728 | AW970937 | Hs.293843 | ESTs | 3.95 | 1232 |
| 432231 | AA339977 | Hs.274127 | CLST 11240 protein | 3.94 | 2647 |
| 432894 | AW167668 | Hs.279772 | brain specific protein | 3.94 | 2709 |
| 435424 | AW083883 | Hs.37896 | Homo sapiens cDNA FLJ13510 fis, clone PL | 3.93 | 2914 |
| 427007 | NM_006283 | Hs.173159 | transforming, acidic coiled-coil contain | 3.91 | 2121 5329 |
| 430803 | AW951830 | Hs.283402 | TCR eta | 3.91 | 2514 |
| 446998 | N99013 | Hs.16762 | Homo sapiens mRNA; cDNA DKFZp564B2062 (f | 3.90 | 3810 |
| 419150 | T29618 | Hs.89640 | TEK tyrosine kinase, endothelial (venous | 3.90 | 1282 |
| 432740 | AF061034 | Hs.278898 | tumor necrosis factor alpha-inducible ce | 3.89 | 2698 5513 |
| 432519 | AI221311 | Hs.130704 | ESTs, Weakly similar to BCHUIA S-100 pro | 3.87 | 2676 |
| 454072 | AW572954 | Hs.252851 | ESTs | 3.87 | 4468 |
| 449261 | AI637592 | | ESTs | 3.86 | 4052 |
| 422195 | AB007903 | Hs.113082 | KIAA0443 gene product | 3.86 | 1614 5141 |
| 411988 | AA455459 | Hs.164480 | ESTs, Weakly similar to T50609 hypotheti | 3.84 | 599 |
| 453467 | AI535997 | Hs.30089 | ESTs | 3.83 | 4410 |
| 409882 | AJ243191 | Hs.56874 | heat shock 27 kD protein family, member 7 | 3.82 | 412 4833 |
| 414577 | AI056548 | Hs.72116 | hypothetical protein FLJ20992 similar to | 3.81 | 847 |
| 427418 | AA402587 | | LAT1-3TM protein | 3.79 | 2152 |
| 441723 | R72137 | Hs.7949 | DKFZP586B2420 protein | 3.77 | 3370 |
| 433614 | W07475 | Hs.277101 | cytochrome c oxidase subunit IV isoform | 3.76 | 2772 |
| 420043 | U08198 | Hs.1285 | complement component 8, gamma polypeptid | 3.74 | 1378 5062 |
| 425887 | AL049443 | Hs.161283 | Homo sapiens mRNA; cDNA DKFZp586N2020 (f | 3.74 | 2010 |
| 442262 | BE170651 | Hs.8700 | deleted in liver cancer 1 | 3.73 | 3408 |
| 447081 | Y13896 | Hs.17287 | potassium inwardly-rectifying channel, s | 3.72 | 3819 |
| 453355 | AW295374 | Hs.31412 | myopodin | 3.72 | 4400 |
| 417033 | H83784 | | KIAA0717 protein | 3.72 | 1055 |
| 458504 | AW070634 | Hs.144794 | ESTs | 3.72 | 4626 |
| 450954 | AI904740 | Hs.25691 | receptor (calcitonin) activity modifying | 3.69 | 4178 |
| 458816 | BE314567 | Hs.211440 | ESTs | 3.68 | 4639 |
| 401234 | | | mitogen-activated protein kinase 8 inter | 3.66 | |
| 456594 | U90545 | Hs.327179 | solute carrier family 17 (sodium phospha | 3.65 | 4553 5801 |
| 435823 | R07856 | Hs.16355 | ESTs | 3.63 | 2948 |
| 415775 | H00747 | Hs.29792 | ESTs, Weakly similar to I38022 hypotheti | 3.63 | 949 |
| 415386 | Z43087 | | gb: HSC13A121 normalized infant brain cDN | 3.62 | 929 |
| 415780 | U75898 | Hs.78846 | heat shock 27 kD protein 2 | 3.61 | 4954 950 |
| 448274 | AI268097 | Hs.67317 | Homo sapiens cDNA FLJ11775 fis, clone HE | 3.61 | 3954 |
| 434951 | AF161442 | Hs.191591 | Homo sapiens HSPC324 mRNA, partial cds | 3.60 | 2882 5559 |
| 425622 | AW360847 | Hs.16578 | ESTs | 3.60 | 1980 |
| 432985 | T92363 | Hs.178703 | ESTs | 3.60 | 2718 |
| 447265 | W23666 | | gb: zb46g08.r1 Soares_fetal_lung_NbHL19W | 3.59 | 3837 |
| 418965 | AI002238 | Hs.11482 | splicing factor, arginine/serine-rich 11 | 3.58 | 1264 |
| 450696 | AI654223 | Hs.16026 | hypothetical protein FLJ23191 | 3.58 | 4150 |
| 414593 | BE386764 | | gb: 601273249F1 NIH_MGC_20 Homo sapiens c | 3.58 | 851 |
| 447742 | AF113925 | Hs.19405 | caspase recruitment domain 4 | 3.57 | 3899 5699 |
| 426828 | NM_000020 | Hs.172670 | activin A receptor type II-like 1 | 3.56 | 2105 5323 |
| 451220 | AF124251 | Hs.26054 | novel SH2-containing protein 3 | 3.56 | 4200 5738 |
| 441503 | AW172263 | Hs.185202 | ESTs | 3.54 | 3355 |
| 447540 | AL135716 | Hs.263780 | ESTs | 3.53 | 3876 |
| 416157 | NM_003243 | Hs.342874 | transforming growth factor, beta recepto | 3.53 | 4956 977 |
| 420983 | W95228 | Hs.100764 | cathepsin G | 3.53 | 1472 |
| 413802 | AW964490 | Hs.32241 | ESTs, Weakly similar to S65657 alpha-1C- | 3.53 | 754 |
| 431971 | BE274907 | Hs.77385 | myosin, light polypeptide 6, alkali, smo | 3.53 | 2622 |
| 411085 | AF022991 | Hs.68398 | period (Drosophila) homolog 1 | 3.52 | 4861 532 |
| 455653 | BE154075 | | gb: PM0-HT0339-200400-010-E05 HT0339 Homo | 3.52 | 4518 |
| 403287 | | | Target Exon | 3.52 | |
| 406760 | AA829363 | | gb: of08g04.s1 NCI_CGAP_Co12 Homo sapiens | 3.50 | 98 |
| 425009 | X58288 | Hs.154151 | protein tyrosine phosphatase, receptor t | 3.49 | 1911 5243 |
| 442321 | AF207664 | Hs.8230 | a disintegrin-like and metalloprotease ( | 3.49 | 3416 5619 |
| 418381 | AA682393 | Hs.119237 | ESTs | 3.48 | 1193 |
| 455698 | BE068097 | | gb: CM1-BT0368-061299-060-c08 BT0368 Homo | 3.47 | 4519 |
| 419360 | AI148037 | | gb: qg61e01.r1 Soares_testis_NHT Homo sap | 3.46 | 1305 |
| 456589 | W79583 | Hs.104258 | Homo sapiens mRNA, exon 1, 2, 3, 4, clon | 3.44 | 4552 |
| 443932 | AW888222 | Hs.9973 | tensin | 3.43 | 3563 |
| 421341 | AJ243212 | | deleted in malignant brain tumors 1 | 3.43 | 1509 5102 |
| 442743 | AI801351 | Hs.302110 | ESTs, Weakly similar to MUC2_HUMAN MUCIN | 3.43 | 3465 |
| 411020 | NM_006770 | Hs.67726 | macrophage receptor with collagenous str | 3.43 | 4860 529 |
| 432485 | N90866 | Hs.276770 | CDW52 antigen (CAMPATH-1 antigen) | 3.43 | 2672 |
| 411962 | AA099050 | | gb: zk85d12.r1 Soares_pregnant_uterus_NbH | 3.42 | 594 |
| 443873 | AL048542 | Hs.16291 | ESTs | 3.42 | 3561 |
| 453874 | AW591783 | Hs.36131 | collagen, type XIV, alpha 1 (undulin) | 3.41 | 4440 |
| 454741 | BE154396 | | gb: CM2-HT0342-091299-050-b05 HT0342 Homo | 3.41 | 4495 |
| 403211 | | | NM_005400*: Homo sapiens protein kinase C | 3.39 | 44 4713 |
| 445107 | AI208121 | Hs.147313 | ESTs, Weakly similar to I38022 hypotheti | 3.39 | 3653 |
| 444769 | AI191650 | Hs.221436 | ESTs | 3.38 | 3626 |
| 448323 | AI492298 | Hs.170915 | ESTs | 3.37 | 3957 |
| 457567 | AW939074 | | gb: QV1-DT0069-010200-057-c12 DT0069 Homo | 3.36 | 4594 |
| 424423 | N56160 | Hs.32916 | nascent-polypeptide-associated complex a | 3.35 | 1843 |
| 451735 | AW407892 | | ESTs | 3.35 | 4244 |
| 421326 | AF051428 | Hs.103504 | estrogen receptor 2 (ER beta) | 3.33 | 1507 5101 |
| 446804 | AW572304 | Hs.271260 | ESTs, Moderately similar to dJ63G5.3 [H. | 3.32 | 3789 |
| 414358 | AA476456 | Hs.142614 | ESTs | 3.32 | 815 |
| 430212 | AA469153 | | gb: nc67f04.s1 NCI_CGAP_Pr1 Homo sapiens | 3.31 | 2455 |
| 436061 | AI248584 | Hs.190745 | Homo sapiens cDNA: FLJ21326 fis, clone C | 3.31 | 2961 |
| 414424 | BE296309 | Hs.125902 | ubiquitin specific protease 2 | 3.31 | 826 |
| 440288 | D81015 | Hs.125382 | ESTs | 3.31 | 3278 |
| 414121 | AA151719 | Hs.95834 | ESTs | 3.30 | 790 |
| 410118 | AW590680 | | von Willebrand factor | 3.28 | 448 |
| 433633 | AI880516 | Hs.84630 | ESTs, Weakly similar to 2004399A chromos | 3.28 | 2775 |
| 417302 | BE245812 | Hs.8941 | ESTs | 3.28 | 1078 |
| 417451 | AW007280 | Hs.115537 | putative dipeptidase | 3.27 | 1096 |
| 448515 | H68441 | Hs.13528 | hypothetical protein FLJ14054 | 3.27 | 3979 |
| 423196 | AK001866 | Hs.125139 | hypothetical protein FLJ11004 | 3.26 | 1726 5173 |
| 404179 | | | Target Exon | 3.26 | |
| 413591 | AA130636 | Hs.188677 | ESTs | 3.26 | 738 |
| 447560 | AF065214 | Hs.18858 | phospholipase A2, group IVC (cytosolic, | 3.24 | 3879 5692 |
| 417075 | R54506 | Hs.118795 | hypothetical protein FLJ10008 | 3.24 | 1061 |
| 456855 | AF035528 | Hs.153863 | MAD (mothers against decapentaplegic, Dr | 3.23 | 4564 5802 |
| 421952 | AA300900 | Hs.98849 | dynein light chain 2B (DNLC2B) | 3.22 | 1584 |
| 434190 | AA627122 | Hs.147644 | ESTs | 3.21 | 2819 |
| 452816 | AA131789 | Hs.61509 | ESTs | 3.21 | 4346 |
| 406702 | Z20656 | | myosin, heavy polypeptide 6, cardiac mus | 3.21 | 4752 90 |
| 415126 | D60945 | | gb: HUM141D04B Clontech human fetal brain | 3.21 | 909 |
| 439484 | AW970218 | Hs.135254 | Homo sapiens clone 1 thrombospondin mRNA | 3.21 | 3211 |
| 429283 | R13008 | Hs.276748 | ESTs | 3.21 | 2349 |
| 433688 | AA628467 | Hs.112572 | Homo sapiens cDNA FLJ14130 fis, clone MA | 3.21 | 2780 |
| 437275 | AW976035 | Hs.292396 | ESTs, Weakly similar to A47582 B-cell gr | 3.20 | 3054 |
| 429580 | AA346839 | Hs.209100 | DKFZP434C171 protein | 3.19 | 2390 |
| 424580 | AA446539 | Hs.339024 | ESTs, Weakly similar to A46010 X-linked | 3.19 | 1860 |
| 426309 | AI912555 | Hs.157195 | peptide YY, 2 (seminalplasmin) | 3.19 | 2049 |
| 438411 | H91928 | Hs.169370 | gb: ys81c10.r1 Soares retina N2b4HR Homo | 3.18 | 3137 |
| 406329 | | | Target Exon | 3.18 | |
| 422686 | BE180557 | Hs.22588 | ESTs | 3.17 | 1674 |
| 447578 | AA912347 | Hs.136585 | ESTs, Weakly similar to JC5314 CDC28/cdc | 3.17 | 3882 |
| 456703 | W07492 | Hs.157101 | ESTs | 3.16 | 4557 |
| 418021 | M15881 | Hs.1137 | uromodulin (uromucoid, Tamm-Horsfall gly | 3.16 | 1147 4999 |
| 447896 | AI436124 | Hs.294069 | Homo sapiens cDNA FLJ13384 fis, clone PL | 3.16 | 3920 |
| 453989 | M63962 | Hs.36992 | ATPase, H? exchanging, alpha polypeptide | 3.16 | 4458 5791 |
| 423488 | AW748714 | Hs.194720 | gb: QV3-BT0294-241199-025-c01 BT0294 Homo | 3.16 | 1751 |
| 417696 | BE241624 | Hs.82401 | CD69 antigen (p60, early T-cell activati | 3.16 | 1113 |
| 420937 | AW966719 | Hs.1340 | colipase, pancreatic | 3.15 | 1466 |
| 443679 | AK001810 | Hs.9670 | hypothetical protein FLJ10948 | 3.15 | 3538 5630 |
| 414325 | AA251929 | | ESTs, Weakly similar to unnamed protein | 3.15 | 812 |
| 459073 | AW968616 | Hs.296234 | ESTs, Weakly similar to T31613 hypotheti | 3.15 | 4655 |
| 428540 | AA430001 | Hs.279755 | ESTs | 3.14 | 2268 |
| 433735 | AA608955 | Hs.109653 | ESTs | 3.14 | 2784 |
| 424455 | AA452006 | Hs.333199 | calcium channel, voltage-dependent, gamm | 3.14 | 1847 |
| 440636 | AA897395 | Hs.202248 | ESTs, Weakly similar to T12525 hypotheti | 3.12 | 3307 |
| 450119 | AI683447 | Hs.264371 | ESTs, Weakly similar to M3K3_HUMAN MITOG | 3.12 | 4105 |
| 414516 | AI307802 | | ESTs, Weakly similar to T43458 hypotheti | 3.11 | 838 |
| 422126 | AW973784 | Hs.112028 | Misshapen/NIK-related kinase | 3.10 | 1605 |
| 450240 | AA236063 | Hs.62954 | ferritin, heavy polypeplide 1 | 3.09 | 4117 |
| 435916 | AW001885 | Hs.114103 | ESTs | 3.08 | 2952 |
| 403875 | | | Target Exon | 3.08 | |
| 440098 | AA864798 | Hs.186180 | Homo sapiens cDNA: FLJ23038 fis, clone L | 3.08 | 3262 |
| 412442 | AI983730 | Hs.26530 | serum deprivation response (phosphatidyl | 3.07 | 631 |
| 428434 | AW363590 | Hs.65551 | Homo sapiens, Similar to DNA segment, Ch | 3.06 | 2256 |
| 452547 | AA335295 | Hs.74120 | adipose specific 2 | 3.05 | 4325 |
| 457661 | AA917801 | Hs.125910 | ESTs | 3.05 | 4598 |
| 407102 | AA007629 | | glycerol-3-phosphate dehydrogenase 1 (so | 3.05 | 123 |
| 438137 | AA778759 | Hs.271690 | ESTs | 3.05 | 3120 |
| 444325 | AW152618 | Hs.16757 | ESTs | 3.05 | 3588 |
| 458871 | AI990292 | Hs.225457 | ESTs | 3.04 | 4642 |
| 426411 | AK000708 | Hs.169764 | hypothetical protein FLJ20701 | 3.04 | 2064 5302 |
| 448782 | AL050295 | | G-protein coupled receptor 116 (GPR116) | 3.04 | 4006 5713 |
| 428224 | X54017 | Hs.183088 | acrosin | 3.04 | 2230 5352 |
| 450749 | AI733114 | Hs.130169 | ESTs | 3.03 | 4160 |
| 407139 | W81598 | | gb: zd88g02.s1 Soares_fetal_heart_NbHH19W | 3.03 | 129 |
| 446932 | AA961459 | Hs.125644 | ESTs | 3.03 | 3802 |
| 422221 | AA306649 | Hs.169370 | FYN oncogene related to SRC, FGR, YES | 3.02 | 1615 |
| 421275 | AW029312 | Hs.99693 | ESTs | 3.02 | 1501 |
| 412351 | AL135960 | Hs.73828 | T-cell acute lymphocytic leukemia 1 | 3.01 | 626 |
| 451556 | AW242829 | Hs.144094 | ESTs, Weakly similar to I38600 zinc fing | 3.01 | 4228 |
| 452664 | AA398859 | Hs.18397 | hypothetical protein FLJ23221 | 3.01 | 4331 |
| 452877 | AI250789 | Hs.32478 | ESTs | 3.00 | 4356 |
| 427890 | AA435761 | | ESTs | 3.00 | 2203 |
| 434666 | AF151103 | Hs.112259 | T cell receptor gamma locus | 3.00 | 2859 5551 |
| 450400 | AI694722 | Hs.279744 | ESTs | 3.00 | 4137 |
| 428287 | AF045239 | Hs.321576 | ring finger protein 22 | 3.00 | 2237 5353 |
| 425023 | AW956889 | Hs.154210 | EDG-1 (endothelial differentiation, sph | 3.00 | 1913 |

TABLE 10B

| Pkey | CAT number | Accession |
|---|---|---|
| 409385 | 110758_1 | T65940 T64515 AA071267 AA071334 |
| 422667 | 224778_1 | AI758223 AW469334 BF940841 AW080348 AI270363 AI055892 |
| | | BE464168 BF431797 BE350144 BF448739 AI693409 BF432999 D62848 AA398070 |
| | | AI383375 AW611490 |
| 439745 | 7961_1 | AL389981 BM023607 BF475999 AI247103 AA838550 AA662305 AA838548 |
| 440647 | 12847_7 | BE397806 BG823998 AI573099 AV725593 AA864452 AA883096 BG059834 |
| | | AV661708 BE561462 BF804447 |
| 423387 | 2612_2 | L13288 AA928785 AI608912 AW872978 AA565655 AI022915 AI304920 AI564366 |
| | | AI668793 AI094557 T60038 R72302 H45409 AA508805 R46356 AA418798 BM129553 |
| | | BM129126 BM129292 BM128865 AI808418 AI689932 AI806573 BF431808 AW872985 |
| | | AW166269 H73241 T16182 AI264547 R73391 R72085 R72840 T83751 X75299 BF754348 |
| | | R94105 AW449839 R73300 NM_004624 AI797007 BE045543 BF110021 BF754250 |
| | | T83923 AW884084 AA903896 AA418962 L20295 R72351 H45098 AA961010 R73210 |
| | | R46451 AW884085 BI022902 BI763932 BI910138 |
| 449261 | 3029652_1 | AI923721 AI860294 AI637592 |
| 427418 | MH1660_94 | AI633280 AW002589 AI760178 AI911270 AI277654 AI184927 AA402587 |
| | | AA402398 AA455831 |
| 417033 | 37085_1 | BC020779 AI857902 AI816715 AI936084 BE044451 AA688029 AW975331 |
| | | AA192427 AA661735 F24489 AI382922 AI659751 F26660 F19507 F31628 AY037148 BF852989 |
| | | BF852995 BF853000 BF853928 BF853920 BF846065 BF846063 BF848191 BF848190 BF853537 |
| | | AA194737 BI010755 H83784 BG203405 BF853580 BF848198 BG545668 BF846059 BI010793 BI010796 |
| | | AI218954 F28028 BF854481 BF846558 AA903899 AA854779 F33888 H83927 F25113 F36903 |
| | | BG214764 AI863957 F26013 F26233 F20527 W37255 W32197 AA192426 AA902491 W33189 |
| | | W32232 BG483879 BF846130 AA661823 |
| 415386 | 11281_5 | R95033 H15506 Z43087 F07410 H54108 H98000 |
| 447265 | 1227845_1 | AW962578 AI370391 W23666 |
| 414593 | 58970_4 | BE386764 AL603555 |
| 455653 | 200977_1 | BF820280 BE154075 BE154018 BE153509 BE153973 BE064861 BE065075 |
| | | BE064684 BE153602 BE153852 BE153847 BE064772 BE064842 BE153557 |
| 406760 | 0_0 | AA829363 |
| 455698 | 1492345_1 | BE068097 BE068148 BE068162 BE068145 BE068200 BE068158 BE068202 |
| | | BE068086 BE068193 BE068091 |
| 419360 | 250410_1 | AA236756 AA287178 AI148037 |
| 421341 | 1407_1 | NM_007329 AF159456 AJ243212 AJ297935 AA295769 NM_017579 |
| | | AJ243224 AI492875 AI796676 AI749838 AA918144 AI814590 AI923531 BF513992 |
| | | AI720725 AI150879 AI279072 AW612904 AI492104 AI284510 AI141231 AA613554 |
| | | AW662148 AW769047 AA565985 AW612888 AU100513 BG955585 BG955588 |
| | | AA295763 BE829414 BF760645 BG954398 AA295332 AA295795 BE932867 |
| | | AW769569 T89953 BE934311 |
| 411962 | 2307710_1 | AA099050 AA099526 T47733 |
| 454741 | 220369_1 | BE154396 BF846839 AW842318 AW817959 BE154393 BI050168 |
| 457567 | 1028609_1 | AW970057 AW939073 AW940012 AW939074 AW939938 AW939206 AW939076 |
| | | AA574383 BE160476 AA573577 AW750479 |
| 451735 | 480028_1 | BG113814 AW407892 AI812096 AW076043 |
| 430212 | 1404178_1 | AI718503 AA469225 AA469153 |
| 410118 | MH1429_162 | AU140255 AW590680 BI051042 R91271 H68390 |
| 406702 | 0_0 | NA |
| 415126 | 1857706_1 | D61346 D60945 D80539 D81568 |
| 414325 | 16029_1 | AK055560 AI223830 AW371603 AA564436 AA251799 |
| | | AA143235 AA975269 AI565174 AI927242 AI052812 AA405571 AW103340 AI289757 |
| | | AI472362 N26127 AI167485 AA826503 AI051406 AI431964 BG434126 AA282258 |
| | | AA808282 BI766582 BE064109 BE064103 BE064088 BE064086 BE064115 BE064066 |
| | | AA282732 |
| 414516 | 60847_1 | AK057782 AI146454 BG703115 AI765980 AI948611 AA889263 AA947457 |
| | | BG547193 H11947 Z38147 F10426 BF447329 H11946 T74968 F06195 BG548563 |
| | | AI004988 AA148735 AI307802 AI439791 BE041453 AI984904 AA148734 F12823 |
| 407102 | 7177_2 | AW945170 BF930905 F33652 BG057818 AI368018 AI421485 AI300352 AI378525 AI264177 |
| | | AI276281 AI245302 AI281050 AI190036 AW451438 AW242903 AA910870 F22289 |
| | | F19647 F22375 AW473816 BF445785 AA774528 F33447 C01077 AW772227 F17759 |
| | | H42812 R09701 AA349096 R48772 H42892 H42537 R47898 N28263 H25721 |
| | | F32386 H43971 R48205 F21390 H45809 AA007629 R47897 R83734 H45844 AW983653 |
| | | H43970 H42536 H24495 R48875 H42961 H22079 R86018 |
| 448782 | 34980_1 | AB018301 AL050295 BF513128 AW385080 AL551708 AI352542 AI829703 AI819388 |
| | | AW629019 AW073189 AW273857 AW118768 AI453845 AI452494 AA886341 AI057144 |
| | | AA904647 AI423547 AW263913 AI094774 AI434419 AI039546 AI002491 AI240412 |
| | | Z25099 AA995178 AW050649 AW026140 AI796309 AI584012 BE166666 AI767991 |
| | | AI309041 AA724059 AI695284 AI245095 T63971 Z40627 BE166681 BG570071 |
| | | BF921915 BI562702 BG506502 AV658066 R48378 AA121543 AI096938 AA618131 |
| | | H40993 R48277 AI352281 BG540263 BG538901 N95226 AI356752 AI221152 Z28777 |
| | | R16574 AW966449 AA044116 AW797518 BI010405 AA044288 AI093508 BE140169 |
| | | T64039 BG433106 AW130367 AW130361 N73937 AA127680 AW044037 AI096437 |
| | | AA384077 BF941499 T93764 BG003285 |
| 427890 | 1373988_1 | AA417099 AA435761 AA972917 AI660387 |

Pkey: Unique Eos probeset identifier number
CAT number: Gene cluster number
Accession: Genbank accession numbers

TABLE 10C

| Pkey | Ref | Strand | Nt_position |
|---|---|---|---|
| 404246 | 7406725 | Plus | 82477-82628, 82721-82817, |
| | | | 82910-83071, 8314 |
| 401974 | 3126777 | Plus | 85330-85683 |
| 403421 | 9665041 | Minus | 126609-126773, 139986-140205 |
| 401929 | 3810670 | Minus | 3167-3286, 4216-4310 |
| 401234 | 9929642 | Plus | 120173-120337 |
| 403287 | 8080320 | Minus | 126097-126411 |
| 403211 | 7630841 | Minus | 159211-159369 |
| 404179 | 4902741 | Plus | 34276-34463 |
| 406329 | 6982072 | Minus | 607903-608271 |
| 403875 | 7709294 | Plus | 28026-28223 |

Pkey: Unique number corresponding to an Eos probeset
Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 11A
About 1198 genes upregulated in breast and lung metastases to the brain relative to normal body tissues

| | |
|---|---|
| Pkey: | Unique Eos probeset identifier number |
| ExAccn: | Exemplar Accession number, Genbank accession number |
| UniGeneID: | UniGene number |
| UniGene Title: | UniGene gene title |
| R1: | 90th percentile of breast and lung metastases to the brain Als divided by the 90th percentile of |
| | normal body tissue Als, where the 15th percentile of all normal body tissue Als was subtracted |
| | from the numerator and denominator. |
| SEQ ID NO(s): | SEQ ID number(s) for nucleic acid and protein sequences associated with table entry. |

| Pkey | ExAccn | UniGeneID | UniGene Title | R1 | SEQ ID NO(S): |
|---|---|---|---|---|---|
| 408591 | AF015224 | Hs.46452 | mammaglobin 1 | 46.40 | 281 4801 |
| 457200 | U33749 | Hs.197764 | thyroid transcription factor 1 | 22.40 | 4582 5812 |
| 414517 | M24461 | Hs.76305 | surfactant, pulmonary-associated protein | 20.52 | 4920 839 |
| 429170 | NM_001394 | Hs.2359 | dual specificity phosphatase 4 | 19.50 | 2336 5392 |
| 419078 | M93119 | Hs.89584 | insulinoma-associated 1 | 19.40 | 1272 5036 |
| 452838 | U65011 | Hs.30743 | preferentially expressed antigen in mela | 15.85 | 4353 5765 |
| 409103 | AF251237 | Hs.112208 | XAGE-1 protein | 15.68 | 333 4812 |
| 426283 | NM_003937 | Hs.169139 | kynureninase (L-kynurenine hydrolase) | 15.20 | 2048 5297 |
| 425057 | AA826434 | Hs.1619 | achaete-scute complex (Drosophila) homol | 14.85 | 1915 |
| 432441 | AW292425 | Hs.163484 | intron of hepatocyte nuclear factor-3 al | 14.40 | 2668 |
| 418738 | AW388633 | Hs.6682 | solute carrier family 7, (cationic amino | 13.85 | 1234 |
| 407276 | AI951118 | Hs.326736 | Homo sapiens breast cancer antigen NY-BR | 13.80 | 147 |
| 443171 | BE281128 | Hs.9030 | TONDU | 13.05 | 3501 |
| 427344 | NM_000869 | Hs.2142 | 5-hydroxytryptamine (serotonin) receptor | 12.25 | 2145 5336 |
| 431164 | AA493650 | Hs.94367 | thyroid transcription factor 1 | 11.50 | 2534 |
| 432415 | T16971 | Hs.289014 | ESTs, Weakly similar to A43932 mucin 2 p | 10.85 | 2665 |
| 457465 | AW301344 | Hs.122908 | DNA replication factor | 10.25 | 4592 |
| 422656 | AI870435 | Hs.1569 | LIM homeobox protein 2 | 10.15 | 1668 |
| 410102 | AW248508 | Hs.279727 | ESTs; homologue of PEM-3 [Ciona savignyi | 10.00 | 446 |
| 409269 | AA576953 | Hs.22972 | steroid 5 alpha-reductase 2-like; H5AR g | 10.00 | 358 |
| 429441 | AJ224172 | Hs.204096 | lipophilin B (uteroglobin family member) | 9.62 | 2371 5407 |
| 453884 | AA355925 | Hs.36232 | KIAA0186 gene product | 9.30 | 4444 |
| 409041 | AB033025 | Hs.50081 | Hypothetical protein, XP_051860 (KIAA119 | 9.15 | 327 4811 |
| 429228 | AI553633 | | hypothetical protein MGC33630 | 8.77 | 2340 |
| 451807 | W52854 | | hypothetical protein FLJ23293 similar to | 8.75 | 4249 |
| 417576 | AA339449 | Hs.82285 | phosphoribosylglycinamide formyltransfer | 8.55 | 1102 |
| 444342 | NM_014398 | Hs.10887 | similar to lysosome-associated membrane | 8.20 | 3591 5638 |
| 447342 | AI199268 | Hs.19322 | Homo sapiens, Similar to RIKEN cDNA 2010 | 8.10 | 3845 |
| 443715 | AI583187 | Hs.9700 | cyclin E1 | 8.05 | 3544 |
| 447033 | AI357412 | Hs.157601 | Predicted gene: Eos cloned; secreted w/V | 8.05 | 3814 |
| 444783 | AK001468 | Hs.62180 | anillin (Drosophila Scraps homolog), act | 7.75 | 3628 5645 |
| 425211 | M18667 | Hs.1867 | progastricsin (pepsinogen C) | 7.67 | 1936 5251 |
| 445413 | AA151342 | Hs.12677 | CGI-147 protein | 7.65 | 3675 |
| 439926 | AW014875 | Hs.137007 | ESTs | 7.36 | 3243 |
| 405770 | | | NM_002362: Homo sapiens melanoma antigen, | 7.34 | 4740 74 |
| 413585 | AI133452 | Hs.75431 | fibrinogen, gamma polypeptide | 7.15 | 737 |
| 404561 | | | trichorhinophalangeal syndrome I gene (T | 7.15 | |
| 439979 | AW600291 | Hs.6823 | hypothetical protein FLJ10430 | 7.12 | 3251 |
| 425843 | BE313280 | Hs.159627 | death associated protein 3 | 7.11 | 2003 |
| 450705 | U90304 | Hs.25351 | iroquois homeobox protein 2A (IRX-2A) ( | 7.10 | 4153 5734 |
| 444670 | H58373 | Hs.332938 | hypothetical protein MGC5370 | 7.10 | 3618 |
| 418164 | AI761820 | Hs.41074 | ESTs, Weakly similar to I39294 McLeod sy | 7.05 | 1165 |
| 421798 | N74880 | | N-acylsphingosine amidohydrolase (acid c | 7.03 | 1564 |
| 448275 | BE514434 | Hs.20830 | kinesin-like 2 | 6.95 | 3955 |
| 428342 | AI739168 | | Homo sapiens cDNA FLJ13458 fis, clone PL | 6.90 | 2244 |
| 409557 | BE182896 | Hs.3686 | ESTs | 6.85 | 384 |
| 400292 | AA250737 | Hs.72472 | BMP-R1B (bone morphogenetic protein rec | 6.75 | 6 |
| 425234 | AW152225 | Hs.165909 | ESTs, Weakly similar to I38022 hypotheti | 6.72 | 1940 |
| 428771 | AB028992 | Hs.193143 | KIAA1069 protein | 6.65 | 2295 5375 |
| 400205 | | | NM_006265*: Homo sapiens RAD21 (S. pombe) | 6.65 | 1 4680 |
| 416208 | AW291168 | Hs.41295 | ESTs, Weakly similar to MUC2_HUMAN MUCIN | 6.65 | 981 |
| 457211 | AW972565 | Hs.32399 | ESTs, Weakly similar to S51797 vasodilat | 6.63 | 4583 |
| 409420 | Z15008 | Hs.54451 | laminin, gamma 2 (nicein (100 kD), kalini | 6.60 | 368 4824 |
| 448664 | AI879317 | Hs.334691 | splicing factor 3a, subunit 1, 120 kD | 6.55 | 3994 |
| 430300 | U60805 | Hs.238648 | oncostatin M receptor | 6.40 | 2465 5441 |
| 408771 | AW732573 | Hs.47584 | potassium voltage-gated channel, delayed | 6.35 | 305 |
| 401451 | | | NM_004496*: Homo sapiens hepatocyte nucle | 6.33 | 27 4697 |
| 433029 | NM_014322 | Hs.279926 | opsin 3 (encephalopsin) | 6.28 | 2726 5524 |
| 430044 | AA464510 | Hs.152812 | ESTs | 6.25 | 2439 |
| 452259 | AA317439 | Hs.28707 | signal sequence receptor, gamma (translo | 6.20 | 4290 |
| 426471 | M22440 | Hs.170009 | transforming growth factor, alpha | 6.15 | 2068 5305 |
| 428004 | AA449563 | Hs.151393 | glutamate-cynteine ligase, catalytic sub | 6.15 | 2213 |
| 427528 | AU077143 | Hs.179565 | minichromosome maintenance deficient (S. | 6.11 | 2161 |
| 416294 | D86980 | Hs.79170 | KIAA0227 protein | 6.10 | 4958 984 |
| 439963 | AW247529 | Hs.6793 | platelet-activating factor acetylhydrola | 6.06 | 3250 |
| 401519 | | | C15000476*: gi\|12737279\|ref\|XP_012163.1\| | 6.05 | |
| 420281 | AI623693 | Hs.323494 | Predicted cation efflux pump | 6.05 | 1405 |
| 408908 | BE296227 | Hs.250822 | serine/threonine kinase 15 | 6.05 | 317 |
| 436217 | T53925 | Hs.107 | fibrinogen-like 1 | 6.01 | 2968 |
| 415138 | C18356 | Hs.295944 | tissue factor pathway inhibitor 2 | 6.00 | 910 |
| 408045 | AW138959 | Hs.245123 | ESTs | 5.95 | 227 |
| 433675 | AW977653 | Hs.75319 | ribonucleotide reductase M2 polypeptide | 5.95 | 2779 |
| 408298 | AI745325 | Hs.271923 | Homo sapiens cDNA: FLJ22785 fis, clone K | 5.95 | 253 |
| 431548 | AI834273 | Hs.9711 | novel protein | 5.85 | 2564 |
| 407999 | AI126271 | Hs.49433 | ESTs, Weakly similar to YZ28_HUMAN HYPOT | 5.83 | 222 |
| 407378 | AA299264 | Hs.57776 | ESTs, Moderately similar to I38022 hypot | 5.65 | 160 |
| 445903 | AI347487 | Hs.132781 | class I cytokine receptor | 5.65 | 3711 |
| 401866 | | | Target Exon | 5.65 | |
| 458814 | AI498957 | Hs.170861 | ESTs, Weakly similar to Z195_HUMAN ZINC | 5.61 | 4638 |
| 440351 | AF030933 | Hs.7179 | RAD1 (S. pombe) homolog | 5.60 | 3285 5607 |
| 452461 | N78223 | Hs.108106 | transcription factor | 5.60 | 4311 |
| 424140 | Z48051 | Hs.141308 | myelin oligodendrocyte glycoprotein | 5.60 | 1809 5200 |
| 400291 | AA401369 | | downstream of breast cancer antigen NY-B | 5.55 | 5 |
| 429597 | NM_003816 | Hs.2442 | a disintegrin and metalloproteinase doma | 5.51 | 2392 5412 |
| 439580 | AF086401 | Hs.293847 | ESTs, Moderately similar to S65657 alpha | 5.45 | 3220 |
| 439772 | AL365406 | Hs.10268 | Homo sapiens mRNA full length insert cDN | 5.45 | 3234 |
| 443695 | AW204099 | | ESTs, Weakly similar to AF126780 1 retin | 5.45 | 3541 |
| 437834 | AA769294 | | gb: nz36g03.s1 NCI_CGAP_GCB1 Homo sapiens | 5.35 | 3096 |
| 430287 | AW182459 | Hs.125759 | ESTs, Weakly similar to LEU5_HUMAN LEUKE | 5.30 | 2460 |
| 433485 | AI493076 | Hs.306098 | aldo-keto reductase family 1, member C2 | 5.30 | 2766 |
| 423739 | AA398155 | Hs.97600 | ESTs | 5.30 | 1771 |
| 438915 | AA280174 | Hs.285681 | Williams-Beuren syndrome chromosome regi | 5.25 | 3178 |
| 419875 | AA853410 | Hs.93557 | proenkephalin | 5.24 | 1365 |
| 424834 | AK001432 | Hs.153408 | Homo sapiens cDNA FLJ10570 fis, clone NT | 5.23 | 1889 |
| 437915 | AI637993 | Hs.202312 | Homo sapiens clone N11 NTera2D1 teratoca | 5.20 | 3105 |
| 419743 | AW408762 | Hs.5957 | Homo sapiens clone 24416 mRNA sequence | 5.15 | 1356 |
| 427944 | AA417878 | Hs.48401 | ESTs, Moderately similar to ALU8_HUMAN A | 5.15 | 2205 |
| 433023 | AW864793 | | thrombospondin 1 | 5.15 | 2725 |
| 420234 | AW404908 | Hs.96038 | Ric (Drosophila)-like, expressed in many | 5.14 | 1399 |
| 427365 | AI873274 | | downstream of breast cancer antigen NY-B | 5.14 | 2148 |
| 416933 | BE561850 | Hs.80506 | small nuclear ribonucleoprotein polypept | 5.13 | 1040 |
| 421502 | AF111856 | Hs.105039 | solute carrier family 34 (sodium phospha | 5.12 | 1527 5110 |
| 410340 | AW182833 | Hs.112188 | hypothetical protein FLJ13149 | 5.10 | 466 |
| 441031 | AI110684 | Hs.7645 | fibrinogen, B beta polypeptide | 5.10 | 3329 |
| 412140 | AA219691 | Hs.73625 | RAB6 interacting, kinesin-like (rabkines | 5.08 | 613 |
| 422486 | BE514492 | Hs.117487 | gene near HD on 4p16.3 with homology to | 5.05 | 1648 |
| 424905 | NM_002497 | Hs.153704 | NIMA (never in mitosis gene a)-related k | 5.05 | 1898 5237 |
| 433409 | AI278802 | Hs.25661 | ESTs | 5.02 | 2761 |
| 422756 | AA441787 | Hs.119689 | glycoprotein hormones, alpha polypeptide | 5.00 | 1679 |
| 424179 | F30712 | Hs.334573 | Homo sapiens, clone IMAGE: 4285740, mRNA | 5.00 | 1812 |
| 426318 | AA375125 | Hs.147112 | Homo sapiens cDNA: FLJ22322 fis, clone H | 5.00 | 2051 |
| 432378 | AI493046 | Hs.146133 | ESTs | 4.95 | 2662 |
| 416111 | AA033813 | Hs.79018 | chromatin assembly factor 1, subunit A ( | 4.95 | 972 |
| 441377 | BE218239 | Hs.202656 | ESTs | 4.95 | 3349 |
| 426827 | AW067805 | Hs.172665 | methylenetetrahydrofolate dehydrogenase | 4.94 | 2104 |
| 432886 | BE159028 | Hs.279704 | chromatin accessibility complex 1 | 4.93 | 2708 |
| 433862 | D86960 | Hs.3610 | KIAA0205 gene product | 4.93 | 2793 5542 |
| 423242 | AL039402 | Hs.125783 | DEME-6 protein | 4.91 | 1730 |
| 413450 | Z99716 | Hs.75372 | N-acetylgalactosaminidase, alpha- | 4.90 | 4901 723 |
| 403485 | | | C3001813*: gi\|12737279\|ref\|XP_012163.1\|k | 4.90 | |
| 416000 | R82342 | Hs.79856 | ESTs, Weakly similar to S65657 alpha-1C- | 4.90 | 965 |
| 410555 | U92649 | Hs.64311 | a disintegrin and metalloproteinase doma | 4.86 | 4851 492 |
| 415786 | AW419196 | Hs.257924 | hypothetical protein FLJ13782 | 4.85 | 951 |
| 450149 | AW969781 | Hs.132863 | Zic family member 2 (odd-paired Drosophi | 4.85 | 4106 |
| 424800 | AL035588 | Hs.153203 | MyoD family inhibitor | 4.81 | 1888 5232 |
| 423554 | M90516 | Hs.1674 | glutamine-fructose-6-phosphate transamin | 4.80 | 1758 5182 |
| 415989 | AI267700 | | ESTs | 4.80 | 963 |
| 424602 | AK002055 | Hs.151046 | hypothetical protein FLJ11193 | 4.80 | 1863 5219 |
| 442760 | BE075297 | Hs.6614 | ESTs, Weakly similar to A43932 mucin 2 p | 4.80 | 3466 |
| 458098 | BE550224 | | metallothionein 1E (functional) | 4.80 | 4611 |
| 422880 | AF228704 | Hs.193974 | glutathione reductase | 4.75 | 1689 5161 |
| 414343 | AL036166 | Hs.75914 | coated vesicle membrane protein | 4.75 | 814 |
| 441285 | NM_002374 | Hs.167 | microtubule-associated protein 2 | 4.75 | 3343 5616 |
| 401464 | | | histone deacetylase 5 | 4.70 | |
| 412537 | AL031778 | | nuclear transcription factor Y, alpha | 4.70 | 644 |
| 423453 | AW450737 | Hs.128791 | CGI-09 protein | 4.70 | 1748 |
| 437387 | AI198874 | Hs.28847 | AD026 protein | 4.66 | 3062 |
| 452526 | W38537 | Hs.280740 | hypothetical protein MGC3040 | 4.65 | 4322 |
| 424085 | NM_002914 | Hs.139226 | replication factor C (activator 1) 2 (40 | 4.65 | 1803 5198 |
| 413753 | U17760 | Hs.75517 | laminin, beta 3 (nicein (125 kD), kalinin | 4.65 | 4906 750 |
| 407137 | T97307 | | gb: ye53h05.s1 Soares fetal liver spleen | 4.62 | 128 |
| 444858 | AI199738 | Hs.208275 | ESTs, Weakly similar to ALUA_HUMAN !!!! | 4.60 | 3633 |
| 413597 | AW302885 | Hs.117183 | ESTs | 4.60 | 739 |
| 448105 | AW591433 | Hs.298241 | Transmembrane protease, serine 3 | 4.59 | 3943 |
| 446872 | X97058 | Hs.16362 | pyrimidinergic receptor P2Y, G-protein c | 4.52 | 3796 5678 |
| 407756 | AA116021 | Hs.38260 | ubiquitin specific protease 18 | 4.51 | 191 |
| 432731 | R31178 | Hs.287820 | fibronectin 1 | 4.47 | 2695 |
| 421227 | R78581 | Hs.266308 | mosaic serine protease | 4.45 | 1492 |
| 418661 | NM_001949 | Hs.1189 | E2F transcription factor 3 | 4.45 | 1222 5022 |
| 438378 | AW970529 | Hs.86434 | hypothetical protein FLJ21816 | 4.45 | 3136 |
| 447519 | U46258 | Hs.339665 | ESTs | 4.44 | 3873 |
| 423673 | BE003054 | Hs.1695 | matrix metalloproteinase 12 (macrophage | 4.40 | 1765 |
| 404996 | | | Target Exon | 4.40 | |
| 441790 | AW294909 | Hs.132208 | ESTs | 4.40 | 3372 |
| 412970 | AB026436 | Hs.177534 | dual specificity phosphatase 10 | 4.40 | 4890 687 |
| 456508 | AA502764 | Hs.123469 | ESTs, Weakly similar to AF208855 1 BM-01 | 4.37 | 4547 |
| 451752 | AB032997 | | KIAA1171 protein | 4.37 | 4247 5750 |
| 449722 | BE280074 | Hs.23960 | cyclin B1 | 4.36 | 4079 |
| 414706 | AW340125 | Hs.76989 | KIAA0097 gene product | 4.36 | 865 |
| 427712 | AI368024 | Hs.283696 | ESTs | 4.36 | 2187 |
| 421305 | BE397354 | Hs.324830 | diptheria toxin resistance protein requi | 4.33 | 1505 |
| 429922 | Z97630 | Hs.226117 | H1 histone family, member 0 | 4.33 | 2427 5430 |
| 444172 | BE147740 | | ESTs, Moderately similar to I38022 hypot | 4.32 | 3580 |
| 429610 | AB024937 | Hs.211092 | LUNX protein; PLUNC (palate lung and nas | 4.32 | 2395 5415 |
| 453439 | AI572438 | Hs.32976 | guanine nucleotide binding protein 4 | 4.31 | 4406 |
| 407366 | AF026942 | Hs.17518 | gb: Homo sapiens cig33 mRNA, partial sequ | 4.30 | 156 |
| 402802 | | | NM_001397: Homo sapiens endothelin conver | 4.30 | 38 4707 |
| 402408 | | | NM_030920*: Homo sapiens hypothetical pro | 4.30 | 33 4703 |
| 438321 | AA576635 | Hs.6153 | CGI-48 protein | 4.29 | 3133 |
| 409435 | AI810721 | Hs.95424 | ESTs | 4.27 | 370 |
| 446440 | AV658411 | Hs.42656 | KIAA1681 protein | 4.25 | 3751 |
| 432350 | NM_005865 | Hs.274407 | protease, serine, 16 (thymus) | 4.25 | 2658 5507 |
| 419926 | AW900992 | Hs.93796 | DKFZP586D2223 protein | 4.22 | 1371 |
| 407235 | D20569 | Hs.169407 | SAC2 (suppressor of actin mutations 2, y | 4.21 | 140 |
| 432140 | AK000404 | Hs.272688 | hypothetical protein FLJ20397 | 4.20 | 2634 5500 |
| 428479 | Y00272 | Hs.334562 | cell division cycle 2, G1 to S and G2 to | 4.20 | 2264 5363 |
| 413582 | AW295647 | Hs.71331 | hypothetical protein MGC5350 | 4.20 | 736 |
| 421928 | AF013758 | Hs.109643 | polyadenylate binding protein-interactin | 4.19 | 1580 5129 |
| 428728 | NM_016625 | Hs.191381 | hypothetical protein | 4.18 | 2288 5373 |
| 424001 | W67883 | Hs.137476 | paternally expressed 10 | 4.17 | 1788 |
| 441362 | BE614410 | Hs.23044 | RAD51 (S. cerevisiae) homolog (E coli Re | 4.17 | 3347 |
| 427658 | H61387 | Hs.30868 | nogo receptor | 4.17 | 2175 |
| 450325 | AI935962 | Hs.91973 | ESTs | 4.16 | 4129 |
| 452279 | AA286844 | | hypothetical protein FLJ13164 | 4.15 | 4293 |
| 408035 | NM_006242 | Hs.42215 | protein phosphatase 1, regulatory subuni | 4.14 | 226 4787 |
| 414696 | AF002020 | Hs.76918 | Niemann-Pick disease, type C1 | 4.10 | 4924 864 |
| 422576 | BE548555 | Hs.118554 | CGI-83 protein | 4.10 | 1659 |
| 401558 | | | ENSP00000220478*: SECRETOGRANIN III. | 4.10 | |
| 431585 | BE242803 | Hs.262823 | hypothetical protein FLJ10326 | 4.10 | 2572 |
| 456977 | AK000252 | Hs.169758 | hypothetical protein FLJ20245 | 4.10 | 4572 5807 |
| 423493 | AI815965 | Hs.129683 | ubiquitin-conjugating enzyme E2D 1 (homo | 4.09 | 1753 |
| 428654 | NM_012091 | Hs.188661 | adenosine deaminase, tRNA-specific 1 | 4.07 | 2280 5370 |
| 416402 | NM_000715 | Hs.1012 | complement component 4-binding protein, | 4.05 | 4959 993 |
| 433077 | AA314262 | Hs.289008 | YDD19 protein | 4.05 | 2732 |
| 430454 | AW469011 | Hs.105635 | ESTs | 4.05 | 2487 |
| 440074 | AA863045 | Hs.10669 | ESTs, Weakly similar to T00050 hypotheti | 4.04 | 3259 |
| 433447 | U29195 | Hs.3281 | neuronal pentraxin II | 4.04 | 2764 5536 |
| 409799 | D11928 | Hs.76845 | phosphoserine phosphatase-like | 4.03 | 407 |
| 436291 | BE568452 | Hs.344037 | protein regulator of cytokinesis 1 | 4.03 | 2975 |
| 407244 | M10014 | | fibrinogen, gamma polypeptide | 4.01 | 143 4767 |
| 452256 | AK000933 | Hs.28661 | Homo sapiens cDNA FLJ10071 fis, clone HE | 4.00 | 4289 |
| 411305 | BE241596 | Hs.69547 | myelin basic protein | 3.99 | 546 |
| 418113 | AI272141 | Hs.83484 | SRY (sex determining region Y)-box 4 | 3.96 | 1161 |
| 423175 | W27595 | Hs.347310 | hypothetical protein FLJ14627 | 3.95 | 1724 |
| 433388 | AI432672 | Hs.288539 | hypothetical protein FLJ22191 | 3.93 | 2759 |
| 440006 | AK000517 | Hs.6844 | NALP2 protein; PYRIN-Containing APAF1-li | 3.91 | 3252 5601 |
| 450193 | AI916071 | Hs.15607 | Homo sapiens Fanconi anemia complementat | 3.91 | 4111 |
| 439453 | BE264974 | Hs.6566 | thyroid hormone receptor interactor 13 | 3.91 | 3208 |
| 400277 | | | Eos Control | 3.90 | |
| 416498 | U33632 | Hs.79351 | potassium channel, subfamily K, member 1 | 3.90 | 4962 999 |
| 446999 | AA151520 | | hypothetical protein MGC4485 | 3.90 | 3811 |
| 407355 | AA846203 | Hs.193974 | ESTs, Weakly similar to ALU1_HUMAN ALU S | 3.90 | 155 |
| 432201 | AI538613 | Hs.298241 | Transmembrane protease, serine 3 | 3.87 | 2643 |
| 408670 | AF160967 | Hs.46784 | potassium large conductance calcium-acti | 3.85 | 291 4803 |
| 409151 | AA306105 | Hs.50785 | SEC22, vesicle trafficking protein (S. c | 3.85 | 341 |
| 422956 | BE545072 | Hs.122579 | ECT2 protein (Epithelial cell transformi | 3.85 | 1698 |
| 426501 | AW043782 | Hs.293616 | ESTs | 3.85 | 2072 |
| 430387 | AW372884 | Hs.240770 | nuclear cap binding protein subunit 2, 2 | 3.83 | 2479 |
| 421437 | AW821252 | Hs.104336 | hypothetical protein | 3.82 | 1519 |
| 426451 | AI908165 | Hs.169946 | GATA-binding protein 3 (T-cell receptor | 3.81 | 2066 |
| 418329 | AW247430 | Hs.84152 | cystathionine-beta-synthase | 3.80 | 1186 |
| 428227 | AA321649 | Hs.2248 | small inducible cytokine subfamily B (Cy | 3.80 | 2231 |
| 404632 | | | NM_022490: Homo sapiens hypothetical prot | 3.80 | 4726 59 |
| 411704 | AI499220 | Hs.71573 | hypothetical protein FLJ10074 | 3.80 | 571 |
| 404571 | | | NM_015902*: Homo sapiens progestin induce | 3.80 | 4724 57 |
| 434834 | AF156774 | Hs.324020 | 1-acylglycerol-3-phosphate O-acyltransfe | 3.77 | 2872 5557 |
| 441553 | AA281219 | Hs.121296 | ESTs | 3.76 | 3357 |
| 407300 | AA102616 | Hs.120769 | gb: zn43e07.s1 Stratagene HeLa cell s3 93 | 3.75 | 149 |
| 411598 | BE336654 | Hs.70937 | H3 histone family, member A | 3.75 | 562 |
| 453900 | AW003582 | Hs.226414 | ESTs, Weakly similar to ALU8_HUMAN ALU S | 3.75 | 4448 |
| 418304 | AA215702 | | gb: zr97g10.r1 NCI_CGAP_GCB1 Homo sapiens | 3.75 | 1178 |
| 425842 | AI587490 | Hs.159623 | NK-2 (Drosophila) homolog B | 3.75 | 2002 |
| 405558 | | | Target Exon | 3.75 | |
| 438869 | AF075009 | | gb: Homo sapiens full length insert cDNA | 3.74 | 3171 |
| 400409 | AF153341 | | Homo sapiens winged helix/forkhead trans | 3.73 | 15 4687 |
| 432840 | AK001403 | Hs.279521 | hypothetical protein FLJ20530 | 3.72 | 2704 5516 |
| 406685 | M18728 | | gb: Human nonspecific crossreacting antig | 3.70 | 4745 83 |
| 415621 | AI648602 | Hs.55468 | ESTs | 3.70 | 938 |
| 443687 | F13040 | | KIAA1228 protein | 3.70 | 3540 |
| 421340 | F07783 | Hs.1369 | decay accelerating factor for complement | 3.70 | 1508 |
| 422440 | NM_004812 | Hs.116724 | aldo-keto reductase family 1, member B10 | 3.68 | 1647 5151 |
| 415339 | NM_015156 | Hs.78398 | KIAA0071 protein | 3.67 | 4946 927 |
| 409757 | NM_001898 | Hs.123114 | cystatin SN | 3.65 | 403 4832 |
| 420390 | AA330047 | Hs.191187 | ESTs | 3.65 | 1418 |
| 442772 | AW503680 | Hs.5957 | Homo sapiens clone 24416 mRNA sequence | 3.65 | 3468 |
| 414670 | BE409525 | Hs.902 | neurofibromin 2 (bilateral acoustic neur | 3.65 | 860 |
| 416198 | H27332 | Hs.99598 | hypothetical protein MGC5338 | 3.65 | 980 |
| 425782 | U66468 | Hs.159525 | cell growth regulatory with EF-hand doma | 3.65 | 1996 5278 |
| 417601 | NM_014735 | Hs.82292 | KIAA0215 gene product | 3.63 | 1105 4991 |
| 435854 | AJ278120 | Hs.4996 | putative ankyrin-repeat containing prote | 3.63 | 2949 5571 |
| 448826 | AI580252 | Hs.293246 | ESTs, Weakly similar to putative p150 [H | 3.62 | 4012 |
| 425242 | D13635 | Hs.155287 | KIAA0010 gene product | 3.61 | 1942 |
| 408452 | AA054683 | Hs.192455 | ESTs, Weakly similar to ALU7_HUMAN ALU S | 3.60 | 268 |
| 434808 | AF155108 | Hs.256150 | NY-REN-41 antigen | 3.60 | 2868 5554 |
| 418478 | U38945 | Hs.1174 | cyclin-dependent kinase inhibitor 2A (me | 3.60 | 1204 5017 |
| 429687 | Al675749 | Hs.211608 | nucleoporin 153 kD | 3.56 | 2406 |
| 431448 | AL137517 | Hs.306201 | hypothetical protein DKFZp564O1278 | 3.55 | 2555 5476 |
| 417378 | R57256 | Hs.82037 | TATA box binding protein (TBP)-associate | 3.55 | 1088 |
| 453160 | AI263307 | | H2B histone family, member L | 3.55 | 4380 |
| 422094 | AF129535 | Hs.272027 | F-box only protein 5 | 3.55 | 1602 5137 |
| 438118 | AW753311 | | ESTs | 3.55 | 3119 |
| 442861 | AA243837 | Hs.57787 | ESTs | 3.55 | 3475 |
| 448186 | AA262105 | Hs.4094 | Homo sapiens cDNA FLJ14208 fis, clone NT | 3.55 | 3949 |
| 432329 | NM_002962 | Hs.2960 | S100 calcium-binding protein A5 | 3.55 | 2655 5506 |
| 449318 | AW236021 | Hs.78531 | Homo sapiens, Similar to RIKEN cDNA 5730 | 3.55 | 4055 |
| 443450 | N66045 | Hs.133529 | ESTs | 3.55 | 3524 |
| 422168 | AA586894 | Hs.112408 | S100 calcium-binding protein A7 (psorias | 3.55 | 1612 |
| 409542 | AA503020 | Hs.36563 | hypothetical protein FLJ22418 | 3.54 | 382 |
| 423250 | BE061916 | Hs.125849 | chromosome 8 open reading frame 2 | 3.54 | 1732 |
| 447349 | AI375546 | | gb: tc23d04.x1 Soares_total_fetus_Nb2HF8_{—} | 3.52 | 3848 |
| 424273 | W40460 | Hs.144442 | phospholipase A2, group X | 3.52 | 1823 |
| 444735 | BE019923 | Hs.243122 | hypothetical protein FLJ13057 similar to | 3.51 | 3623 |
| 453785 | AI368236 | Hs.283732 | ESTs, Moderately similar to ALU1_HUMAN A | 3.50 | 4432 |
| 406081 | | | Target Exon | 3.50 | |
| 411096 | U80034 | Hs.68583 | mitochondrial intermediate peptidase | 3.50 | 4862 535 |
| 409902 | AI337658 | Hs.156351 | ESTs | 3.50 | 416 |
| 458621 | AI221741 | Hs.117777 | ESTs | 3.49 | 4630 |
| 435664 | AI032087 | Hs.269819 | ESTs | 3.48 | 2936 |
| 438714 | AA814859 | | ESTs | 3.46 | 3161 |
| 415947 | U04045 | Hs.78934 | mutS (E. coli) homolog 2 (colon cancer, | 3.46 | 4955 962 |
| 409731 | AA125985 | Hs.56145 | thymosin, beta, identified in neuroblast | 3.45 | 402 |
| 453941 | U39817 | Hs.36820 | Bloom syndrome | 3.45 | 4454 5790 |
| 442353 | BE379594 | Hs.49136 | ESTs, Moderately similar to ALU7_HUMAN A | 3.45 | 3423 |
| 410206 | NM_003826 | Hs.60415 | N-ethylmaleimide-sensitive factor attach | 3.45 | 454 4840 |
| 408527 | AL135018 | Hs.33074 | Homo sapiens, clone IMAGE: 3606519, mRNA, | 3.44 | 276 |
| 415079 | R43179 | Hs.22895 | hypothetical protein FLJ23548 | 3.44 | 906 |
| 420005 | AW271106 | Hs.133294 | ESTs | 3.43 | 1372 |
| 425354 | U62027 | Hs.155935 | complement component 3a receptor 1 | 3.43 | 1952 5257 |
| 442118 | AA976718 | Hs.202242 | ESTs | 3.40 | 3399 |
| 434844 | AF157116 | Hs.22350 | hypothetical protein LOC56757 | 3.40 | 2873 |
| 426931 | NM_003416 | Hs.2076 | zinc finger protein 7 (KOX 4, clone HF.1 | 3.40 | 2114 5328 |
| 415263 | AA948033 | Hs.130853 | ESTs | 3.40 | 920 |
| 412863 | AA121673 | Hs.59757 | zinc finger protein 281 | 3.40 | 679 |
| 454453 | AW752781 | | hypothetical protein FLJ12614 similar to | 3.38 | 4485 |
| 441128 | AA570256 | | ESTs, Weakly similar to T23273 hypotheti | 3.38 | 3334 |
| 422938 | NM_001809 | Hs.1594 | centromere protein A (17 kD) | 3.38 | 1694 5162 |
| 443180 | R15875 | Hs.258576 | claudin 12 | 3.37 | 3504 |
| 428839 | AI767756 | Hs.82302 | Homo sapiens cDNA FLJ14814 fis, clone NT | 3.37 | 2310 |
| 411605 | AW006831 | | ESTs | 3.37 | 563 |
| 413719 | BE439580 | Hs.75498 | small inducible cytokine subfamily A (Cy | 3.36 | 749 |
| 444371 | BE540274 | Hs.239 | forkhead box M1 | 3.35 | 3592 |
| 430188 | AL049242 | Hs.234794 | Homo sapiens mRNA; cDNA DKFZp564B083 (fr | 3.35 | 2453 |
| 408101 | AW968504 | Hs.123073 | CDC2-related protein kinase 7 | 3.35 | 234 |
| 425769 | U72513 | Hs.159486 | Human RPL13-2 pseudogene mRNA, complete | 3.35 | 1995 |
| 451806 | NM_003729 | Hs.27076 | RNA 3'-terminal phosphate cyclase | 3.35 | 4248 5751 |
| 410166 | AK001376 | Hs.59346 | hypothetical protein FLJ10514 | 3.35 | 451 4839 |
| 442326 | H92962 | Hs.124813 | hypothetical protein MGC14817 | 3.35 | 3417 |
| 442660 | AW138174 | Hs.130651 | ESTs | 3.35 | 3458 |
| 446054 | AB014537 | Hs.13604 | KIAA0637 gene product | 3.34 | 3722 5664 |
| 427581 | NM_014788 | Hs.179703 | KIAA0129 gene product | 3.33 | 2167 5339 |
| 433556 | W56321 | Hs.111460 | calcium/calmodulin-dependent protein kin | 3.33 | 2768 |
| 448552 | AW973653 | Hs.20104 | hypothetical protein FLJ00052 | 3.33 | 3983 |
| 434203 | BE262677 | Hs.283558 | hypothetical protein PRO1855 | 3.33 | 2820 |
| 402496 | | | Target Exon | 3.32 | |
| 432169 | Y00971 | Hs.2910 | phosphoribosyl pyrophosphate synthetase | 3.30 | 2637 5501 |
| 410174 | AA306007 | Hs.59461 | DKFZP434C245 protein | 3.30 | 453 |
| 424332 | AA338919 | Hs.101615 | ESTs | 3.30 | 1830 |
| 412530 | AA766268 | Hs.266273 | hypothetical protein FLJ13346 | 3.30 | 643 |
| 425018 | BE245277 | Hs.154196 | E4F transcription factor 1 | 3.30 | 1912 |
| 430187 | AI799909 | Hs.158989 | ESTs | 3.30 | 2452 |
| 440138 | AB033023 | Hs.318127 | hypothetical protein FLJ10201 | 3.30 | 3267 5603 |
| 444743 | AA045648 | Hs.301957 | nudix (nucleoside diphosphate linked moi | 3.30 | 3624 |
| 415992 | C05837 | Hs.145807 | hypothetical protein FLJ13593 | 3.30 | 964 |
| 400297 | AI127076 | Hs.306201 | hypothetical protein DKFZp564O1278 | 3.29 | 9 |
| 442108 | AW452649 | Hs.166314 | ESTs | 3.28 | 3398 |
| 410762 | AF226053 | Hs.66170 | HSKM-B protein | 3.26 | 4857 514 |
| 420552 | AK000492 | Hs.98806 | hypothetical protein | 3.25 | 1430 5081 |
| 442152 | R39246 | Hs.239666 | Homo sapiens cDNA FLJ13495 fis, clone PL | 3.25 | 3401 |
| 422420 | U03398 | Hs.1524 | tumor necrosis factor (ligand) superfami | 3.25 | 1643 5150 |
| 435532 | AW291488 | Hs.117305 | Homo sapiens, clone IMAGE: 3682908, mRNA | 3.25 | 2923 |
| 436643 | AA757626 | Hs.10941 | ESTs, Weakly similar to IPP1_HUMAN PROTE | 3.25 | 2999 |
| 433183 | AF231338 | Hs.222024 | transcription factor BMAL2 | 3.25 | 2745 5531 |
| 427521 | AW973352 | | ESTs | 3.25 | 2159 |
| 411945 | AL033527 | Hs.92137 | L-myc-2 protein (MYCL2) | 3.25 | 4870 590 |
| 444381 | BE387335 | Hs.283713 | hypothetical protein BC014245 | 3.24 | 3593 |
| 430335 | D80007 | Hs.239499 | KIAA0185 protein | 3.24 | 2473 5448 |
| 438525 | AW368528 | Hs.100855 | ESTs | 3.24 | 3145 |
| 400247 | | | Eos Control | 3.23 | |
| 427621 | BE621182 | Hs.179882 | hypothetical protein FLJ12443 | 3.22 | 2173 |
| 418338 | NM_002522 | Hs.84154 | neuronal pentraxin I | 3.21 | 1189 5010 |
| 448686 | AA158659 | Hs.334712 | hypothetical protein FLJ14744 | 3.20 | 3997 |
| 450728 | AW162923 | Hs.25363 | presenilin 2 (Alzheimer disease 4) | 3.20 | 4156 |
| 426581 | AB040956 | Hs.135890 | KIAA1523 protein | 3.20 | 2080 5311 |
| 442179 | AA983842 | Hs.333555 | chromosome 2 open reading frame 2 | 3.20 | 3404 |
| 422536 | AA311915 | Hs.187726 | gb: EST182621 Jurkat T-cells VI Homo sapi | 3.20 | 1654 |
| 449704 | AK000733 | Hs.23900 | GTPase activating protein | 3.20 | 4076 5722 |
| 448595 | AB014544 | Hs.21572 | KIAA0644 gene product | 3.20 | 3987 5711 |
| 442592 | BE566623 | Hs.29899 | ESTs, Weakly similar to G02075 transcrip | 3.19 | 3450 |
| 452994 | AW962597 | Hs.31305 | KIAA1547 protein | 3.19 | 4363 |
| 426699 | AA383337 | Hs.121269 | ESTs | 3.18 | 2089 |
| 427719 | AI393122 | Hs.134726 | ESTs | 3.17 | 2189 |
| 418836 | AI655499 | Hs.161712 | BMP-R1B | 3.17 | 1247 |
| 410619 | BE512730 | Hs.65114 | keratin 18 | 3.16 | 498 |
| 414271 | AK000275 | Hs.75871 | protein kinase C binding protein 1 | 3.15 | 807 |
| 452833 | BE559681 | Hs.30736 | KIAA0124 protein | 3.15 | 4351 |
| 452827 | AI571835 | Hs.55468 | ESTs | 3.14 | 4350 |
| 430462 | AI584156 | Hs.105640 | Homo sapiens, clone IMAGE: 4139775, mRNA, | 3.14 | 2488 |
| 449145 | AI632122 | Hs.198408 | ESTs | 3.14 | 4039 |
| 453902 | BE502341 | Hs.3402 | ESTs | 3.13 | 4449 |
| 420982 | AW576160 | Hs.100729 | KIAA0692 protein | 3.13 | 1471 |
| 425529 | NM_014656 | Hs.158282 | KIAA0040 gene product | 3.13 | 1975 5270 |
| 430289 | AK001952 | Hs.238039 | hypothetical protein FLJ11090 | 3.13 | 2461 5440 |
| 413413 | D82520 | | zinc finger protein 36 (KOX 18) | 3.11 | 717 |
| 432728 | NM_006979 | Hs.278721 | HLA class II region expressed gene KE4 | 3.10 | 2694 5512 |
| 420090 | AA220238 | Hs.94986 | ribonuclease P (38 kD) | 3.10 | 1383 |
| 446715 | AI337735 | Hs.173919 | ESTs, Moderately similar to ZN91_HUMAN Z | 3.10 | 3778 |
| 424539 | L02911 | Hs.150402 | Activin A receptor, type I (ACVR1) (ALK | 3.10 | 1857 5216 |
| 443054 | AI745185 | Hs.84520 | yes-associated protein 65 kDa | 3.10 | 3491 |
| 425641 | D79758 | Hs.14355 | Homo sapiens cDNA FLJ13207 fis, clone NT | 3.10 | 1981 |
| 423642 | AW452650 | Hs.157148 | hypothetical protein MGC13204 | 3.10 | 1763 |
| 422005 | BE266556 | Hs.110702 | Homo sapiens mRNA; cDNA DKFZp761E212 (fr | 3.10 | 1592 |
| 426108 | AA622037 | Hs.166468 | programmed cell death 5 | 3.08 | 2028 |
| 415632 | U67085 | Hs.78524 | TcD37 homolog | 3.08 | 4950 939 |
| 426227 | U67058 | Hs.154299 | Human proteinase activated receptor-2 mR | 3.07 | 2041 |
| 425649 | U30930 | Hs.158540 | UDP glycosyltransferase 8 (UDP-galactose | 3.07 | 1982 5273 |
| 447541 | AK000288 | Hs.18800 | hypothetical protein FLJ20281 | 3.07 | 3877 5691 |
| 433323 | AA805132 | Hs.159142 | ESTs | 3.07 | 2755 |
| 420522 | AW957137 | Hs.98541 | hypothetical protein | 3.07 | 1427 |
| 450256 | AA286887 | Hs.24724 | MFH-amplified sequences with leucine-ric | 3.06 | 4120 |
| 429978 | AA249027 | | ribosomal protein S6 | 3.06 | 2433 |
| 403011 | | | ENSP00000215330*: Probable serine/threoni | 3.05 | |
| 401203 | | | Target Exon | 3.05 | |
| 417975 | AA641836 | Hs.30085 | hypothetical protein FLJ23186 | 3.05 | 1143 |
| 410507 | AA355288 | | transitional epithelia response protein | 3.05 | 486 |
| 404440 | | | NM_021048: Homo sapiens melanoma antigen, | 3.05 | 4721 54 |
| 419865 | NM_007020 | Hs.93502 | U1-snRNP binding protein homolog (70 kD) | 3.05 | 1364 5058 |
| 410561 | BE540255 | Hs.6994 | Homo sapiens cDNA: FLJ22044 fis, clone H | 3.05 | 493 |
| 430066 | AI929659 | Hs.237825 | signal recognition particle 72 kD | 3.05 | 2442 |
| 425322 | U63630 | Hs.155637 | protein kinase, DNA-activated, catalytic | 3.05 | 1950 5255 |
| 412715 | NM_000947 | Hs.74519 | primase, polypeptide 2A (58 kD) | 3.04 | 4885 662 |
| 442530 | AI580830 | Hs.176508 | Homo sapiens cDNA FLJ14712 fis, clone NT | 3.04 | 3437 |
| 406690 | M29540 | Hs.220529 | carcinoembryonic antigen-related cell ad | 3.03 | 4748 86 |
| 424163 | NM_007071 | Hs.142245 | HERV-H LTR-associating 3 | 3.03 | 1811 5201 |
| 450447 | AF212223 | Hs.25010 | hypothetical protein P15-2 | 3.03 | 4139 5730 |
| 447444 | AK000318 | Hs.18616 | hypothetical protein FLJ20311 | 3.02 | 3862 5689 |
| 429903 | AL134197 | Hs.93597 | cyclin-dependent kinase 5, regulatory su | 3.02 | 2425 |
| 435472 | AW972330 | Hs.283022 | triggering receptor expressed on myeloid | 3.01 | 2917 |
| 426572 | AB037783 | Hs.170623 | hypothetical protein FLJ11183 | 3.00 | 2079 5310 |
| 426215 | AW963419 | Hs.155223 | stanniocalcin 2 | 3.00 | 2039 |
| 441102 | AA973905 | | intermediate filament protein syncoilin | 3.00 | 3333 |
| 444165 | AL137443 | Hs.10441 | hypothetical protein FLJ11236 | 3.00 | 3579 |
| 425673 | R70318 | Hs.339730 | ESTs | 3.00 | 1985 |
| 442619 | AA447492 | Hs.20183 | ESTs, Weakly similar to AF164793 1 prote | 3.00 | 3455 |
| 413832 | AI913647 | Hs.131886 | Homo sapiens cDNA: FLJ22113 fis, clone H | 3.00 | 755 |
| 421515 | Y11339 | Hs.105352 | GalNAc alpha-2,6-sialyltransferase I, I | 3.00 | 1532 5113 |
| 420610 | AI683183 | Hs.99348 | distal-less homeo box 5 | 3.00 | 1437 |
| 401435 | | | C14000397*: gi\|7499898\|pir∥T33295 hypoth | 3.00 | 26 |
| 422771 | NM_012318 | Hs.120165 | leucine zipper-EF-hand containing transm | 3.00 | 1681 5158 |
| 454293 | H49739 | Hs.134013 | ESTs, Moderately similar to HK61_HUMAN H | 2.99 | 4475 |
| 425465 | L18964 | Hs.1904 | protein kinase C, iota | 2.98 | 1969 5266 |
| 429183 | AB014604 | Hs.197955 | KIAA0704 protein | 2.97 | 2337 5393 |
| 448789 | BE539108 | Hs.22051 | hypothetical protein MGC15548 | 2.97 | 4007 |
| 429084 | AJ001443 | Hs.195614 | splicing factor 3b, subunit 3, 130 kD | 2.97 | 2332 5390 |
| 431197 | AL038596 | Hs.250745 | polymerase (RNA) III (DNA directed) (62 k | 2.96 | 2539 |
| 436167 | AA705651 | Hs.25087 | ESTs | 2.96 | 2965 |
| 418203 | X54942 | Hs.83758 | CDC28 protein kinase 2 | 2.96 | 1169 5003 |
| 451071 | AW138807 | Hs.204939 | ESTs, Highly similar to A54677 homeotic | 2.95 | 4189 |
| 420362 | U79734 | Hs.97206 | huntingtin interacting protein 1 | 2.95 | 1417 5075 |
| 442875 | BE623003 | Hs.23625 | Homo sapiens clone TCCCTA00142 mRNA sequ | 2.95 | 3477 |
| 437389 | AL359587 | Hs.271586 | hypothetical protein DKFZp762M115 | 2.94 | 3063 5586 |
| 418941 | AA452970 | Hs.239527 | E1B-55 kDa-associated protein 5 | 2.94 | 1260 |
| 439375 | AA689526 | Hs.344249 | steroid dehydrogenase homolog | 2.94 | 3204 |
| 411975 | AI916058 | Hs.144583 | 3′UTR of: dead ringer (Drosophila)-like | 2.93 | 596 |
| 439574 | AI469788 | | ESTs | 2.93 | 3219 |
| 417348 | AI940507 | Hs.318526 | hypothetical protein FLJ12661 | 2.93 | 1084 |
| 447155 | AA100605 | Hs.121557 | ESTs, Weakly similar to DP1_HUMAN POLYPO | 2.93 | 3827 |
| 420956 | AA351584 | Hs.100543 | Homo sapiens clone 24505 mRNA sequence | 2.92 | 1468 |
| 419705 | AW368634 | Hs.154331 | ESTs | 2.92 | 1351 |
| 425159 | NM_004341 | Hs.154868 | carbamoyl-phosphate synthetase 2, aspart | 2.92 | 1931 5249 |
| 416902 | AA375634 | Hs.288974 | hypothetical protein FLJ12528 | 2.91 | 1035 |
| 407242 | M18728 | | gb: Human nonspecific crossreacting antig | 2.91 | 142 4766 |
| 446880 | AI811807 | Hs.108646 | Homo sapiens cDNA FLJ14934 fis, clone PL | 2.91 | 3799 |
| 406581 | | | Target Exon | 2.90 | |
| 404607 | | | Target Exon | 2.90 | |
| 401093 | | | C12000586*: gi\|6330167\|dbj\|BAA86477.1\| (A | 2.90 | |
| 421247 | BE391727 | Hs.102910 | general transcription factor IIH, polype | 2.90 | 1495 |
| 405366 | | | NM_003371*: Homo sapiens vav 2 oncogene ( | 2.90 | 4735 69 |
| 418700 | AI963808 | Hs.86970 | ESTs, Moderately similar to ALU5_HUMAN A | 2.90 | 1230 |
| 417288 | AI984792 | Hs.108812 | hypothetical protein FLJ22004 | 2.90 | 1077 |
| 419752 | AA249573 | Hs.152618 | ESTs, Moderately similar to ZN91_HUMAN Z | 2.90 | 1358 |
| 450375 | AA009647 | | a disintegrin and metalloproteinase doma | 2.90 | 4133 |
| 419497 | NM_006410 | Hs.90753 | Tat-interacting protein (30 kD) | 2.90 | 1320 5050 |
| 423849 | AL157425 | Hs.133315 | Homo sapiens mRNA; cDNA DKFZp761J1324 (f | 2.90 | 1780 |
| 452909 | NM_015368 | Hs.30985 | pannexin 1 | 2.90 | 4358 5767 |
| 430294 | AI538226 | Hs.32976 | guanine nucleotide binding protein 4 | 2.90 | 2463 |
| 414004 | AA737033 | Hs.7155 | similar to thymidylate kinase family LPS | 2.89 | 772 |
| 439680 | AW245741 | Hs.58461 | ESTs, Weakly similar to A35659 krueppel- | 2.89 | 3229 |
| 432908 | AI861896 | | ESTs | 2.89 | 2711 |
| 408296 | AL117452 | Hs.44155 | DKFZP586G1517 protein | 2.88 | 252 4792 |
| 450065 | AL050107 | Hs.24341 | transcriptional co-activator with PDZ-bi | 2.88 | 4102 5725 |
| 431770 | BE221880 | Hs.268555 | 5′-3′ exoribonuclease 2 | 2.87 | 2596 |
| 431933 | AI187057 | Hs.132554 | ESTs | 2.87 | 2618 |
| 424954 | NM_000546 | Hs.1846 | tumor protein p53 (Li-Fraumeni syndrome) | 2.87 | 1901 5238 |
| 423771 | AF053004 | Hs.132781 | class I cytokine receptor | 2.87 | 1773 5186 |
| 449976 | H06350 | Hs.135056 | Human DNA sequence from clone RP5-850E9 | 2.87 | 4095 |
| 410442 | X73424 | Hs.63788 | propionyl Coenzyme A carboxylase, beta p | 2.87 | 479 4848 |
| 423165 | AI937547 | Hs.124915 | hypothetical protein MGC2601 | 2.87 | 1722 |
| 445704 | AI493742 | Hs.167700 | ESTs, Moderately similar to I38022 hypot | 2.86 | 3694 |
| 407198 | H91679 | | gb: yv04a07.s1 Soares fetal liver spleen | 2.86 | 135 |
| 412942 | AL120344 | Hs.75074 | mitogen-activated protein kinase-activat | 2.85 | 685 |
| 432229 | AW290976 | Hs.143587 | ESTs | 2.85 | 2646 |
| 430452 | AI888450 | Hs.174644 | hypothetical protein FLJ21669 | 2.85 | 2486 |
| 422805 | AA436989 | Hs.121017 | H2A histone family, member A | 2.85 | 1683 |
| 443614 | AV655386 | Hs.7645 | fibrinogen, B beta polypeptide | 2.85 | 3532 |
| 429370 | C19097 | Hs.89709 | glutamate-cysteine ligase, modifier subu | 2.85 | 2361 |
| 428829 | R14050 | Hs.194051 | Homo sapiens mRNA; cDNA DKFZp566B213 (fr | 2.84 | 2306 |
| 411285 | AI733766 | Hs.69429 | Homo sapiens IMAGE: 512024 clone, mRNA | 2.84 | 545 |
| 415402 | AA164687 | Hs.177576 | mannosyl (alpha-1,3-)-glycoprotein beta- | 2.84 | 930 |
| 414821 | M63835 | Hs.77424 | Fc fragment of IgG, high affinity la, re | 2.83 | 4929 881 |
| 446595 | T57448 | Hs.15467 | hypothetical protein FLJ20725 | 2.83 | 3768 |
| 442643 | U82756 | | PRP4/STK/WD splicing factor | 2.83 | 3457 5623 |
| 428438 | NM_001955 | Hs.2271 | endothelin 1 | 2.82 | 2257 5358 |
| 441024 | AW081530 | Hs.268231 | ESTs | 2.82 | 3327 |
| 453005 | AW055308 | Hs.31803 | ESTs, Weakly similar to N-WASP [H. sapien | 2.82 | 4365 |
| 420851 | AA281062 | Hs.29493 | hypothetical protein FLJ20142 | 2.82 | 1460 |
| 412482 | AI499930 | Hs.334885 | mitochondrial GTP binding protein | 2.81 | 636 |
| 408633 | AW963372 | Hs.46677 | PRO2000 protein | 2.81 | 286 |
| 421077 | AK000061 | Hs.101590 | hypothetical protein | 2.81 | 1479 5093 |
| 433730 | AK002135 | Hs.3542 | hypothetical protein FLJ11273 | 2.81 | 2783 5540 |
| 433867 | AK000596 | Hs.3618 | hippocalcin-like 1 | 2.81 | 2794 |
| 433701 | AW445023 | Hs.15155 | ESTs | 2.81 | 2782 |
| 425849 | AJ000512 | Hs.296323 | serum/glucocorticoid regulated kinase | 2.81 | 2005 5279 |
| 433285 | AW975944 | Hs.237396 | ESTs | 2.81 | 2753 |
| 407634 | AW016569 | Hs.136414 | UDP-GlcNAc: betaGal beta-1,3-N-acetylgluc | 2.81 | 174 |
| 457819 | AA057484 | Hs.35406 | FLJ20522 Hypothetical protein FLJ20522 | 2.81 | 4601 |
| 426925 | NM_001196 | Hs.315689 | Homo sapiens cDNA: FLJ22373 fis, clone H | 2.80 | 2112 5326 |
| 416406 | D86961 | Hs.79299 | lipoma HMGIC fusion partner-like 2 | 2.80 | 4960 994 |
| 419544 | AI909154 | | gb: QV-BT200-010499-007 BT200 Homo sapien | 2.80 | 1329 |
| 428134 | AA421773 | Hs.161008 | ESTs | 2.80 | 2221 |
| 436211 | AK001581 | Hs.334828 | hypothetical protein FLJ10719; KIAA1794 | 2.80 | 2967 5576 |
| 432006 | AL137382 | Hs.272320 | Homo sapiens mRNA; cDNA DKFZp434L1226 (f | 2.80 | 2626 |
| 428758 | AA433988 | Hs.98502 | CA125 antigen; mucin 16 | 2.80 | 2292 |
| 422358 | AL133030 | Hs.115429 | Homo sapiens mRNA for KIAA1666 protein, | 2.79 | 1635 5149 |
| 452834 | AI638627 | Hs.105685 | KIAA1688 protein | 2.78 | 4352 |
| 411908 | L27943 | Hs.72924 | cytidine deaminase | 2.78 | 4869 585 |
| 453968 | AA847843 | Hs.62711 | High mobility group (nonhistone chromoso | 2.77 | 4456 |
| 431865 | AA521106 | Hs.136375 | ESTs, Weakly similar to S65824 reverse t | 2.77 | 2610 |
| 435937 | AA830893 | Hs.119769 | ESTs | 2.77 | 2953 |
| 434263 | N34895 | Hs.79187 | ESTs | 2.76 | 2825 |
| 434067 | H18913 | Hs.124023 | Homo sapiens cDNA FLJ14218 fis, clone NT | 2.75 | 2812 |
| 415451 | H19415 | Hs.268720 | ESTs, Moderately similar to ALU1_HUMAN A | 2.75 | 933 |
| 444185 | AW298350 | Hs.113602 | ESTs | 2.75 | 3582 |
| 445873 | AA250970 | Hs.251946 | poty(A)-binding protein, cytoplasmic 1-I | 2.75 | 3707 |
| 411630 | U42349 | Hs.71119 | Putative prostate cancer tumor suppresso | 2.75 | 4866 565 |
| 453775 | NM_002916 | Hs.35120 | replication factor C (activator 1) 4 (37 | 2.75 | 4431 5783 |
| 407771 | AL138272 | Hs.1600 | ESTs | 2.75 | 193 |
| 423492 | AF020761 | Hs.129683 | ubiquitin-conjugating enzyme E2D 1 (homo | 2.75 | 1752 |
| 413063 | AL035737 | Hs.75184 | chitinase 3-like 1 (cartilage glycoprote | 2.74 | 692 |
| 431130 | NM_006103 | Hs.2719 | HE4; epididymis-specific, whey-acidic pr | 2.74 | 2533 5470 |
| 421654 | AW163267 | Hs.106469 | suppressor of var1 (S. cerevisiae) 3-like | 2.74 | 1550 |
| 409430 | R21945 | Hs.346735 | splicing factor, arginine/serine-rich 5 | 2.74 | 369 |
| 438533 | AI440266 | Hs.170673 | ESTs, Weakly similar to T24832 hypotheti | 2.74 | 3146 |
| 443209 | AI040125 | Hs.150521 | ESTs | 2.73 | 3508 |
| 409439 | AW390511 | Hs.288862 | Homo sapiens cDNA: FLJ21260 fis, clone C | 2.73 | 371 |
| 424511 | BE300512 | Hs.193557 | ESTs, Moderately similar to ALU7_HUMAN A | 2.73 | 1854 |
| 443162 | T49951 | Hs.9029 | DKFZP434G032 protein | 2.73 | 3500 |
| 455601 | AI368680 | Hs.816 | SRY (sex determining region Y)-box 2 | 2.73 | 4515 |
| 455286 | BE144384 | | gb: MR0-HT0166-191199-004-c11 HT0166 Homo | 2.73 | 4504 |
| 453751 | R36762 | Hs.101282 | Homo sapiens cDNA: FLJ21238 fis, clone C | 2.73 | 4428 |
| 424308 | AW975531 | Hs.154443 | minichromosome maintenance deficient (S. | 2.73 | 1827 |
| 422963 | M79141 | Hs.13234 | sphingosine 1-phosphate phosphohydrolase | 2.72 | 1701 |
| 426174 | AA547959 | Hs.115838 | Homo sapiens similar to Echinoidin (LOC1 | 2.72 | 2036 |
| 439210 | AA641928 | Hs.194071 | ESTs, Weakly similar to unnamed protein | 2.72 | 3192 |
| 410082 | AA081594 | Hs.158311 | Musashi (Drosophila) homolog 1 | 2.71 | 442 |
| 417454 | NM_000202 | Hs.172458 | iduronate 2-sulfatase (Hunter syndrome) | 2.71 | 1097 4986 |
| 427083 | NM_006363 | Hs.173497 | Sec23 (S. cerevisiae) homolog B | 2.71 | 2124 5330 |
| 448877 | AI583696 | Hs.253313 | ESTs | 2.70 | 4016 |
| 400240 | | | Eon Control | 2.70 | |
| 430881 | NM_000809 | Hs.248112 | gamma-aminobutyric acid (GABA) A recepto | 2.70 | 2520 5465 |
| 443323 | BE560621 | Hs.9222 | estrogen receptor binding site associate | 2.70 | 3518 |
| 432188 | AI362952 | Hs.2928 | solute carrier family 7 (cationic amino | 2.70 | 2641 |
| 455290 | U75810 | | gb: HSU75810 Human Homo sapiens cDNA clon | 2.70 | 4505 |
| 429782 | NM_005754 | Hs.220689 | Ras-GTPase-activating protein SH3-domain | 2.70 | 2416 5425 |
| 456759 | BE259150 | Hs.127792 | delta (Drosophila)-like 3 | 2.70 | 4560 |
| 428816 | AA004986 | Hs.193852 | ATP-binding cassette, sub-family C (CFTR | 2.70 | 2303 |
| 406625 | Y13647 | Hs.119597 | stearoyl-CoA desaturase (delta-9-desatur | 2.70 | 4744 79 |
| 425236 | AW067800 | Hs.155223 | stanniocalcin 2 | 2.69 | 1941 |
| 429048 | AI372949 | Hs.44241 | Homo sapiens cDNA: FLJ21447 fis, clone C | 2.69 | 2329 |
| 404819 | | | NM_002688*: Homo sapiens peanut (Drosophi | 2.68 | 4728 62 |
| 423645 | AI215632 | Hs.147487 | ESTs | 2.68 | 1764 |
| 440327 | R12581 | Hs.191146 | ESTs | 2.67 | 3282 |
| 426746 | J03626 | Hs.2057 | uridine monophosphate synthetase (orotat | 2.67 | 2092 5317 |
| 422283 | AW411307 | Hs.114311 | CDC45 (cell division cycle 45, S. cerevis | 2.67 | 1626 |
| 400880 | | | NM_000611*: Homo sapiens CD59 antigen p18 | 2.66 | 23 4694 |
| 420440 | NM_002407 | Hs.97644 | mammaglobin 2 | 2.66 | 1422 5076 |
| 452099 | BE612992 | Hs.27931 | hypothetical protein FLJ10607 similar to | 2.66 | 4270 |
| 413431 | AW246428 | Hs.75355 | ubiquitin-conjugating enzyme E2N (homolo | 2.66 | 720 |
| 459672 | Z18867 | Hs.326843 | gb: HSDHEGC03 Stratagene cDNA library Hum | 2.65 | 4676 |
| 447805 | AW627932 | Hs.302421 | gemin4 | 2.65 | 3908 |
| 429277 | AW452016 | Hs.127863 | ESTs, Weakly similar to SFR4_HUMAN SPLIC | 2.65 | 2347 |
| 447673 | AI823987 | Hs.182285 | ESTs | 2.65 | 3889 |
| 420210 | AI557257 | Hs.44811 | ESTs | 2.65 | 1395 |
| 449570 | AA001793 | | gb: zh86c06.r1 Soares_fetal_liver_spleen_{—} | 2.65 | 4068 |
| 453878 | AW964440 | Hs.19025 | DC32 | 2.65 | 4442 |
| 436488 | BE620909 | Hs.261023 | hypothetical protein FLJ20958 | 2.65 | 2989 |
| 442275 | AW449467 | Hs.54795 | Homo sapiens secretoglobin, family 3A, m | 2.64 | 3409 |
| 446839 | BE091926 | Hs.16244 | mitotic spindle coiled-coil related prot | 2.64 | 3794 |
| 413278 | BE563085 | Hs.833 | interferon-stimulated protein, 15 kDa | 2.64 | 705 |
| 439235 | AA188827 | Hs.7988 | ESTs, Weakly similar to I38022 hypotheti | 2.64 | 354 |
| 412673 | AL042957 | Hs.31845 | ESTs | 2.64 | 659 |
| 446258 | AI283476 | Hs.263478 | ESTs | 2.64 | 3740 |
| 439211 | AI890347 | Hs.271923 | Homo sapiens cDNA: FLJ22785 fis, clone K | 2.64 | 3193 |
| 415323 | BE269352 | Hs.949 | neutrophil cytosolic factor 2 (65 kD, chr | 2.63 | 926 |
| 409703 | NM_006187 | Hs.56009 | 2′-5′-oligoadenylate synthetase 3 (100 k | 2.63 | 396 4831 |
| 410305 | AF030409 | Hs.62185 | solute carrier family 9 (sodium/hydrogen | 2.63 | 463 4843 |
| 423081 | AF262992 | Hs.123159 | sperm associated antigen 4 | 2.62 | 1717 5167 |
| 458981 | AW968318 | Hs.285996 | hypothetical protein FLJ23375 | 2.62 | 4648 |
| 434540 | NM_016045 | Hs.3945 | CGI-107 protein | 2.60 | 2847 5549 |
| 411190 | AA306342 | Hs.69171 | protein kinase C-like 2 | 2.60 | 539 |
| 453392 | U23752 | Hs.32964 | SRY (sex determining region Y)-box 11 | 2.60 | 4403 5776 |
| 436027 | AI864053 | Hs.39972 | GM2 ganglioside activator protein (GM2A) | 2.60 | 2956 |
| 433906 | AI167816 | Hs.43355 | ESTs | 2.60 | 2796 |
| 415857 | AA866115 | Hs.127797 | Homo sapiens cDNA FLJ11381 fis, clone HE | 2.60 | 956 |
| 432723 | D29677 | Hs.3085 | KIAA0054 gene product; Helicase | 2.60 | 2693 |
| 408031 | AA081395 | Hs.42173 | Homo sapiens cDNA FLJ10366 fis, clone NT | 2.60 | 225 |
| 448663 | BE614599 | | hypothetical protein MGC14797 | 2.60 | 3993 |
| 427700 | AA262294 | Hs.180383 | dual specificity phosphatase 6 | 2.60 | 2185 |
| 437478 | AL390172 | Hs.317432 | branched chain aminotransferase 1, cytos | 2.60 | 3067 |
| 426514 | BE616633 | Hs.170195 | bone morphogenetic protein 7 (osteogenic | 2.60 | 2073 |
| 434826 | AF155661 | Hs.22265 | pyruvate dehydrogenase phosphatase | 2.60 | 2871 5556 |
| 442711 | AF151073 | Hs.8645 | hypothetical protein | 2.59 | 3460 5625 |
| 401197 | | | ENSP00000229263*: HSPC213. | 2.59 | |
| 420802 | U22376 | Hs.1334 | v-myb avian myeloblastosis viral oncogen | 2.59 | 1455 5087 |
| 437967 | BE277414 | Hs.5947 | mel transforming oncogene (derived from | 2.59 | 3112 |
| 411423 | AW845987 | Hs.68864 | ESTs, Weakly similar to phosphatidylseri | 2.58 | 555 |
| 409262 | AK000631 | Hs.52256 | hypothetical protein FLJ20624 | 2.58 | 355 4819 |
| 433009 | AA761668 | | gb: nz24c08.s1 NCI_CGAP_GCB1 Homo sapiens | 2.58 | 2721 |
| 423309 | BE006775 | Hs.126782 | sushi-repeat protein | 2.58 | 1736 |
| 429950 | AW081608 | Hs.105053 | ESTs | 2.58 | 2430 |
| 447334 | AA515032 | Hs.91109 | ESTs | 2.57 | 3844 |
| 412935 | BE267045 | Hs.75064 | tubulin-specific chaperone c | 2.57 | 683 |
| 412123 | BE251328 | Hs.73291 | hypothetical protein FLJ10881 | 2.57 | 610 |
| 421307 | B2539976 | Hs.103305 | Homo sapiens mRNA; cDNA DKFZp434B0425 (f | 2.57 | 1506 |
| 450671 | AI356967 | Hs.43086 | ESTs, Weakly similar to A46010 X-linked | 2.56 | 4147 |
| 451418 | BE387790 | Hs.26369 | hypothetical protein FLJ20287 | 2.56 | 4221 |
| 428450 | NM_014791 | Hs.184339 | KIAA0175 gene product | 2.55 | 2259 5359 |
| 412141 | AI183838 | Hs.48938 | hypothetical protein FLJ21802 | 2.55 | 614 |
| 404580 | | | trichorhinophalangeal syndrome I gene (T | 2.55 | |
| 444461 | R53734 | Hs.25978 | ESTs, Weakly similar to 2109260A B cell | 2.55 | 3600 |
| 428264 | AA424839 | Hs.98484 | ESTs, Weakly similar to ALU1_HUMAN ALU S | 2.55 | 2234 |
| 413374 | NM_001034 | Hs.75319 | ribonucleotide reductase M2 polypeptide | 2.55 | 4899 713 |
| 436009 | H57130 | Hs.120925 | ESTs | 2.55 | 2955 |
| 421433 | AI829192 | Hs.22380 | ESTs | 2.55 | 1518 |
| 441224 | AU076964 | Hs.7753 | calumenin | 2.54 | 3338 |
| 447197 | R36075 | | gb: yh88b01.s1 Soares placenta Nb2HP Homo | 2.54 | 3828 |
| 457579 | AB030816 | Hs.36761 | HRAS-like suppressor | 2.54 | 4595 5813 |
| 410660 | AI061118 | Hs.65328 | Fanconi anemia, complementation group F | 2.54 | 502 |
| 447064 | AB002350 | Hs.17262 | KIAA0352 gene product | 2.54 | 3816 5680 |
| 419507 | AW170425 | Hs.87680 | ESTs | 2.53 | 1322 |
| 422095 | AI868872 | Hs.282804 | hypothetical protein FLJ22704 | 2.53 | 1603 |
| 449845 | AW971183 | Hs.6019 | DnaJ (Hsp40) homolog, subfamily C, membe | 2.53 | 4088 |
| 443426 | AF098158 | Hs.9329 | chromosome 20 open reading frame 1 | 2.53 | 3523 5627 |
| 440273 | AI805392 | Hs.325335 | Homo sapiens cDNA: FLJ23523 fis, clone L | 2.53 | 3274 |
| 440340 | AW895503 | Hs.125276 | ESTs | 2.53 | 3284 |
| 418867 | D31771 | Hs.89404 | msh (Drosophila) homeo box homolog 2 | 2.53 | 1252 5032 |
| 428484 | AF104032 | Hs.184601 | solute carrier family 7 (cationic amino | 2.53 | 2265 5364 |
| 432586 | AA568548 | | ESTs | 2.53 | 2681 |
| 406922 | S70284 | Hs.119597 | gb: stearoyl-CoA desaturase [human, adipo | 2.52 | 109 4755 |
| 424581 | M62062 | Hs.150917 | catenin (cadherin-associated protein), a | 2.52 | 1861 |
| 408393 | AW015318 | Hs.23165 | ESTs | 2.52 | 263 |
| 408949 | AF189011 | Hs.49163 | putative ribonuclease III | 2.52 | 319 4809 |
| 449207 | AL044222 | Hs.23255 | nucleoporin 155 kD | 2.52 | 4045 |
| 449444 | AW818436 | | solute carrier family 16 (monocarboxylic | 2.52 | 4062 |
| 441373 | AI266421 | Hs.120179 | Homo sapiens cDNA: FLJ22133 fis, clone H | 2.51 | 3348 |
| 451380 | H09280 | Hs.13234 | ESTs | 2.51 | 4218 |
| 448641 | R31845 | Hs.21666 | insulin-like 4 (placenta) | 2.51 | 3991 |
| 428844 | AW972635 | Hs.301904 | hypothetical protein FLJ12671 | 2.51 | 2311 |
| 424259 | AK001776 | Hs.143954 | hypothetical protein FLJ10914 | 2.50 | 1822 5207 |
| 442053 | R35343 | Hs.24968 | Human DNA sequence from clone RP1-233G16 | 2.50 | 3391 |
| 402102 | | | Target Exon | 2.50 | |
| 409648 | AW451449 | Hs.57749 | ESTs | 2.50 | 391 |
| 416677 | T83470 | Hs.334840 | ESTs, Moderately similar to I78885 serin | 2.50 | 1012 |
| 446251 | AW867156 | Hs.282589 | ESTs, Weakly similar to I38022 hypotheti | 2.50 | 3739 |
| 413472 | BE242870 | Hs.75379 | solute carrier family 1 (glial high affi | 2.50 | 724 |
| 400298 | AA032279 | Hs.61635 | six transmembrane epithelial antigen of | 2.50 | 10 |
| 420807 | AA280627 | Hs.57846 | ESTs | 2.50 | 1457 |
| 451722 | H86374 | Hs.40861 | ESTs | 2.50 | 4243 |
| 438523 | H66220 | Hs.278177 | ESTs | 2.50 | 3144 |
| 402812 | | | NM_004930*: Homo sapiens capping protein | 2.49 | 39 4708 |
| 426991 | AK001536 | Hs.214410 | Homo sapiens cDNA FLJ10674 fis, clone NT | 2.49 | 2117 |
| 452092 | BE245374 | Hs.27842 | hypothetical protein FLJ11210 | 2.49 | 4268 |
| 407881 | AW072003 | Hs.40968 | heparan sulfate (glucosamine) 3-O-sulfot | 2.49 | 211 |
| 402855 | | | NM_001839*: Homo sapiens calponin 3, acid | 2.49 | 40 4709 |
| 422809 | AK001379 | Hs.121028 | hypothetical protein FLJ10549 | 2.49 | 1684 |
| 410444 | W73484 | Hs.132554 | gb: zd54e04.s1 Soares_fetal_heart_NbHH19W | 2.48 | 480 |
| 457292 | AI921270 | Hs.281462 | hypothetical protein FLJ14251 | 2.48 | 4586 |
| 408990 | AL022395 | Hs.49526 | f-box and leucine-rich repeat protein 4 | 2.48 | 321 4810 |
| 441085 | AW136551 | Hs.181245 | Homo sapiens cDNA FLJ12532 fis, clone NT | 2.48 | 3331 |
| 449209 | BE616830 | Hs.294145 | ESTs | 2.48 | 4046 |
| 439352 | BE614347 | Hs.169615 | hypothetical protein FLJ20989 | 2.48 | 3202 |
| 411979 | X85134 | Hs.72984 | retinoblastoma-binding protein 5 | 2.48 | 4872 597 |
| 434474 | AL042936 | Hs.211571 | holocytochrome c synthase (cytochrome c | 2.47 | 2842 |
| 431566 | AF176012 | Hs.260720 | J domain containing protein 1 | 2.47 | 2568 5479 |
| 453507 | AF083217 | Hs.33085 | WD repeat domain 3 | 2.47 | 4414 5778 |
| 432540 | AI821517 | Hs.105866 | ESTs | 2.47 | 2678 |
| 420085 | AI741909 | Hs.44680 | hypothetical protein FLJ20979 | 2.47 | 1381 |
| 408761 | AA057264 | Hs.238936 | ESTs, Weakly similar to (define not ava | 2.47 | 304 |
| 417018 | M16038 | Hs.80887 | v-yes-1 Yamaguchi sarcoma viral related | 2.47 | 1053 4976 |
| 428878 | AA436884 | Hs.48926 | ESTs | 2.47 | 2315 |
| 443257 | AI334040 | Hs.11614 | HSPC065 protein | 2.47 | 3514 |
| 410503 | AW975746 | Hs.188662 | KIAA1702 protein | 2.47 | 485 |
| 411248 | AA551538 | Hs.69321 | Homo sapiens cDNA FLJ14408 fis, clone HE | 2.46 | 541 |
| 416984 | H38765 | Hs.80706 | diaphorase (NADH/NADPH) (cytochrome b-5 | 2.46 | 1047 |
| 414844 | AA296874 | Hs.77494 | deoxyguanosine kinase | 2.46 | 884 |
| 452436 | BE077546 | Hs.31447 | ESTs, Moderately similar to A46010 X-lin | 2.46 | 4308 |
| 445266 | BE222118 | Hs.12479 | associated molecule with the SH3 domain | 2.45 | 3665 |
| 422363 | T55979 | Hs.115474 | replication factor C (activator 1) 3 (38 | 2.45 | 1636 |
| 445994 | NM_004724 | Hs.13512 | ZW10 (Drosophila) homolog, centromere/ki | 2.45 | 3716 5661 |
| 410595 | AW629223 | Hs.64794 | zinc finger protein 183 (RING finger, C3 | 2.45 | 495 |
| 438578 | AA811244 | | ESTs | 2.45 | 3151 |
| 437748 | AF234882 | Hs.5814 | suppression of tumorigenicity 7 | 2.45 | 3087 5591 |
| 411402 | BE297855 | Hs.69855 | NRAS-related gene | 2.45 | 552 |
| 406671 | AA129547 | Hs.285754 | met proto-oncogene (hepatocyte growth fa | 2.45 | 82 |
| 418882 | NM_004996 | Hs.89433 | ATP-binding cassette, sub-family C (CFTR | 2.45 | 1254 5033 |
| 424081 | NM_006413 | Hs.139120 | tibonuclease P (30 kD) | 2.45 | 1801 5197 |
| 439492 | AF086310 | Hs.103159 | ESTs | 2.45 | 3212 |
| 409617 | BE003760 | Hs.55209 | Homo sapiens mRNA; cDNA DKFZp434K0514 (f | 2.45 | 387 |
| 413545 | AA766632 | Hs.119451 | ESTs | 2.45 | 732 |
| 422765 | AW409701 | Hs.1578 | baculoviral IAP repeat-containing 5 (sur | 2.45 | 1680 |
| 426251 | M24283 | Hs.168383 | intercellular adhesion molecule 1 (CD54) | 2.45 | 2043 5296 |
| 428862 | NM_000346 | Hs.2316 | SRY (sex determining region Y)-box 9 (ca | 2.45 | 2313 5382 |
| 439955 | AW203959 | Hs.149532 | ESTs | 2.45 | 3249 |
| 431958 | X63629 | Hs.2877 | cadherin 3, type 1, P-cadherin (placenta | 2.45 | 2621 5498 |
| 421768 | AI923934 | Hs.108112 | histone fold protein CHRAC17; DNA polyme | 2.44 | 1560 |
| 410575 | BE207480 | Hs.6994 | Homo sapiens cDNA: FLJ22044 fis, clone H | 2.44 | 494 |
| 413551 | BE242639 | Hs.75425 | ubiquitin associated protein | 2.44 | 733 |
| 438956 | W00847 | Hs.135056 | Human DNA sequence from clone RP5-850E9 | 2.44 | 3180 |
| 418340 | NM_013286 | Hs.84162 | chromosome 3p21.1 gene sequence | 2.44 | 1190 5011 |
| 441318 | AI078234 | | ESTs | 2.44 | 3345 |
| 417386 | AL037228 | Hs.82043 | D123 gene product | 2.44 | 1090 |
| 428970 | BE276891 | Hs.194691 | retinoic acid induced 3 (RAIG1); metabo | 2.44 | 2321 |
| 448072 | AI459306 | Hs.24908 | ESTs | 2.43 | 3940 |
| 447397 | BE247676 | Hs.18442 | E-1 enzyme | 2.43 | 3856 |
| 441659 | BE564162 | Hs.250820 | hypothetical protein FLJ14827 | 2.43 | 3366 |
| 440590 | AI863446 | Hs.266308 | mosaic serine protease | 2.43 | 3301 |
| 422309 | U79745 | Hs.114924 | solute carrier family 16 (monocarboxylic | 2.43 | 1630 5146 |
| 414747 | U30872 | Hs.77204 | centromere protein F (350/400 kD, mitosin | 2.42 | 4927 873 |
| 442961 | BE614474 | | F-box only protein 22 | 2.42 | 3484 |
| 446950 | AA305800 | Hs.5672 | hypothetical protein AF140225 | 2.42 | 3806 |
| 421462 | AF016495 | Hs.104624 | aquaporin 9 | 2.42 | 1522 5108 |
| 450251 | BE080483 | | gb: QV1-BT0630-280200-086-a05 BT0630 Homo | 2.42 | 4118 |
| 450277 | AI690071 | Hs.283552 | ESTs, Weakly similar to unnamed protein | 2.42 | 4122 |
| 422150 | AI867118 | | calpastatin | 2.41 | 1609 |
| 425371 | D49441 | Hs.155981 | mesothelin | 2.41 | 1957 5259 |
| 452826 | BE245286 | Hs.301636 | peroxisomal biogenesis factor 6 | 2.41 | 4349 |
| 439708 | AI761369 | Hs.59584 | hypothetical protein FLJ21144 | 2.41 | 3231 |
| 422744 | AW268803 | Hs.119640 | hBKLF for basic kruppel like factor | 2.41 | 1678 |
| 412783 | BE276738 | Hs.74578 | DEAD/H (Asp-Glu-Ala-Asp/His) box polypep | 2.41 | 669 |
| 418526 | BE019020 | Hs.85838 | solute carrier family 16 (monocarboxylic | 2.40 | 1211 |
| 452960 | AK001335 | Hs.31137 | protein tyrosine phosphatase, receptor t | 2.40 | 4360 |
| 414368 | W70171 | Hs.75939 | uridine monophosphate kinase | 2.40 | 818 |
| 409892 | AW956113 | Hs.7149 | gb: EST368183 MAGE resequences, MAGD Homo | 2.40 | 414 |
| 442991 | BE281238 | Hs.8886 | hypothetical protein FLJ20424 | 2.40 | 3485 |
| 425216 | U81504 | Hs.155172 | adaptor-related protein complex 3, beta | 2.40 | 1938 5252 |
| 443829 | AI087954 | Hs.23348 | S-phase kinase-associated protein 2 (p45 | 2.40 | 3557 |
| 442072 | AI740832 | Hs.12311 | Homo sapiens clone 23570 mRNA sequence | 2.40 | 3395 |
| 412193 | AI684467 | Hs.144057 | ESTs | 2.40 | 617 |
| 408826 | AF216077 | Hs.48376 | Homo sapiens clone HB-2 mRNA sequence | 2.40 | 309 |
| 422631 | BE218919 | Hs.118793 | hypothetical protein FLJ10688 | 2.40 | 1665 |
| 422981 | AF026445 | Hs.122752 | TATA box binding protein (TBP)-associate | 2.40 | 1706 5165 |
| 418330 | BE409405 | | ESTs | 2.40 | 1187 |
| 434627 | AI221894 | Hs.39311 | ESTs | 2.40 | 2853 |
| 418067 | AI127958 | Hs.83393 | cystatin E/M | 2.40 | 1156 |
| 442426 | AI373062 | Hs.332938 | hypothetical protein MGC5370 | 2.40 | 3427 |
| 433561 | BE540937 | Hs.20104 | hypothetical protein FLJ00052 | 2.39 | 2769 |
| 422336 | AI761322 | Hs.115285 | dihydrolipoamide S-acetyltransferase (E2 | 2.39 | 1633 |
| 431374 | BE258532 | Hs.251871 | CTP synthase | 2.39 | 2551 |
| 437271 | AL137445 | Hs.28846 | Homo sapiens mRNA; cDNA DKFZp566O134 (fr | 2.39 | 3052 |
| 409974 | BE174106 | Hs.225641 | hypothetical protein FLJ13171 | 2.39 | 423 |
| 445921 | AW015211 | Hs.146181 | ESTs | 2.39 | 3712 |
| 431310 | AW327889 | Hs.252433 | Homo sapiens cDNA FLJ13794 fis, clone TH | 2.39 | 2545 |
| 435124 | AA725362 | Hs.120456 | ESTs | 2.38 | 2897 |
| 401405 | | | Target Exon | 2.38 | |
| 452234 | AW084176 | Hs.223296 | ESTs, Weakly similar to I38022 hypotheti | 2.38 | 4284 |
| 415214 | AI445236 | Hs.125124 | EphB2 | 2.38 | 917 |
| 404592 | | | NM_022739*: Homo sapiens E3 ubiquitin lig | 2.38 | 4725 58 |
| 434378 | AA631739 | Hs.335440 | EST | 2.38 | 2836 |
| 406203 | | | Target Exon | 2.38 | |
| 442355 | AA456539 | Hs.8262 | lysosomal-associated membrane protein 2 | 2.38 | 3424 |
| 425548 | AA890023 | Hs.1906 | prolactin receptor | 2.38 | 1978 |
| 409132 | AJ224538 | Hs.50732 | protein kinase, AMP-activated, beta 2 no | 2.37 | 338 4813 |
| 404661 | | | C9000306*: gi\|12737280\|ref\|XP_006682.2\| k | 2.37 | |
| 431127 | U66618 | Hs.250581 | SWI/SNF related, matrix associated, acti | 2.37 | 2532 |
| 453905 | NM_002314 | Hs.36566 | LIM domain kinase 1 | 2.37 | 4450 5788 |
| 416114 | AI695549 | Hs.183868 | glucuronidase, beta | 2.37 | 973 |
| 413437 | BE313164 | Hs.75361 | gene from NF2/meningioma region of 22q12 | 2.37 | 722 |
| 418403 | D86978 | Hs.84790 | KIAA0225 protein | 2.37 | 1197 5015 |
| 419092 | J05581 | Hs.89603 | mucin 1, transmembrane | 2.37 | 1275 5038 |
| 434094 | AA305599 | Hs.238205 | hypothetical protein PRO2013 | 2.36 | 2814 |
| 450800 | BE395161 | Hs.1390 | proteasome (prosome, macropain) subunit, | 2.36 | 4167 |
| 452323 | W44356 | Hs.292812 | ESTs, Weakly similar to T33468 hypotheti | 2.36 | 4296 |
| 411678 | AI907114 | Hs.71465 | squalene epoxidase | 2.35 | 568 |
| 414774 | X02419 | Hs.77274 | plasminogen activator, urokinase | 2.35 | 4928 877 |
| 421379 | Y15221 | Hs.103982 | small inducible cytokine subfamily B (Cy | 2.35 | 1515 5104 |
| 448030 | N30714 | Hs.325960 | membrane-spanning 4-domains, subfamily A | 2.35 | 3936 |
| 454645 | AW811928 | | gb: RC2-ST0168-240300-017-b02 ST0168 Homo | 2.35 | 4493 |
| 425368 | AB014595 | Hs.155976 | cullin 4B | 2.35 | 1956 5258 |
| 425212 | AW962253 | Hs.171618 | ESTs | 2.35 | 1937 |
| 426098 | NM_014906 | Hs.166351 | KIAA1072 protein | 2.35 | 2026 5291 |
| 432281 | AK001239 | Hs.274263 | hypothetical protein FLJ10377 | 2.35 | 2652 5505 |
| 418592 | X99226 | Hs.284153 | Fanconi anemia, complementation group A | 2.35 | 1215 5020 |
| 412043 | BE156622 | Hs.333371 | Homo sapiens clone TA40 untranslated mRN | 2.35 | 604 |
| 431696 | AA259068 | Hs.267819 | protein phosphatase 1, regulatory (inhib | 2.35 | 2584 |
| 451065 | AW295132 | Hs.222231 | ESTs, Weakly similar to granule cell mar | 2.35 | 4188 |
| 416018 | AW138239 | Hs.78977 | proprotein convertase subtilisin/kexin t | 2.35 | 966 |
| 432226 | AW182766 | Hs.273558 | phosphate cytidylyltransferase 1, cholin | 2.35 | 2645 |
| 425048 | H05468 | Hs.164502 | ESTs | 2.35 | 1914 |
| 409187 | AF154830 | Hs.50966 | carbamoyl-phosphate synthetase 1, mitoch | 2.35 | 347 4816 |
| 414629 | AA345824 | Hs.76688 | carboxylesterase 1 (monocyte/macrophage | 2.35 | 855 |
| 413010 | AA393273 | Hs.75133 | transcription factor 6-like 1 (mitochond | 2.35 | 689 |
| 416426 | AA180256 | Hs.210473 | Homo sapiens cDNA FLJ14872 fis, clone PL | 2.35 | 996 |
| 441021 | AW578716 | Hs.7644 | H1 histone family, member 2 | 2.35 | 3326 |
| 435005 | U80743 | Hs.306094 | trinucleotide repeat containing 12 | 2.35 | 2886 5560 |
| 407204 | R41933 | Hs.140237 | ESTs, Weakly similar to ALU1_HUMAN ALU S | 2.35 | 136 |
| 445654 | X91247 | Hs.13046 | thioredoxin reductase 1 | 2.34 | 3691 5658 |
| 437682 | AA476652 | Hs.94952 | Homo sapiens cDNA: FLJ23371 fis, clone H | 2.34 | 3083 |
| 417333 | AL157545 | Hs.173179 | bromodomain and PHD finger containing, 3 | 2.34 | 1081 |
| 417831 | H16423 | Hs.82685 | CD47 antigen (Rh-related antigen, integr | 2.34 | 1128 |
| 453204 | R10799 | Hs.191990 | ESTs | 2.34 | 4385 |
| 430791 | AA486293 | Hs.272068 | ESTs, Weakly similar to ALU3_HUMAN ALU S | 2.34 | 2513 |
| 415752 | BE314524 | Hs.78776 | putative transmembrane protein | 2.34 | 948 |
| 432788 | AA521091 | Hs.178499 | Homo sapiens cDNA: FLJ23117 fis, clone L | 2.34 | 2699 |
| 429857 | AF089897 | Hs.294030 | topoisomerase-related function protein 4 | 2.33 | 2419 5428 |
| 453857 | AL080235 | Hs.35861 | Ras-induced senescence 1 (RIS1) | 2.33 | 4437 5785 |
| 418372 | AA311833 | Hs.84318 | replication protein A1 (70 kD) | 2.33 | 1191 |
| 424755 | AB033094 | Hs.152925 | KIAA1268 protein | 2.33 | 1882 5231 |
| 432623 | AA557351 | Hs.152448 | ESTs, Moderately similar to S14147 multi | 2.33 | 2686 |
| 434815 | AF155582 | Hs.46744 | core1 UDP-galactose: N-acetylgalactosamin | 2.32 | 2870 5555 |
| 431183 | NM_006855 | Hs.250696 | KDEL (Lys-Asp-Glu-Leu) endoplasmic retic | 2.32 | 2537 5472 |
| 409196 | NM_001874 | Hs.334873 | carboxypeptidase M | 2.32 | 350 4817 |
| 426272 | AW450671 | Hs.189284 | ESTs | 2.31 | 2046 |
| 447644 | AW861622 | Hs.108646 | Homo sapiens cDNA FLJ14934 fis, clone PL | 2.31 | 3886 |
| 429276 | AF056085 | Hs.198612 | G protein-coupled receptor 51 | 2.31 | 2346 5397 |
| 456497 | AW967956 | Hs.123648 | ESTs, Weakly similar to AF108460 1 ubinu | 2.30 | 4546 |
| 439778 | AL109729 | Hs.99364 | putative transmembrane protein | 2.30 | 3235 |
| 446237 | AW270515 | Hs.149596 | Homo sapiens, Similar to RIKEN cDNA 2310 | 2.30 | 3738 |
| 405411 | | | ENSP00000252213: SODIUM BICARBONATE COTRA | 2.30 | |
| 426262 | AI792141 | Hs.196270 | folate transporter/carrier | 2.30 | 2045 |
| 400639 | | | C10000999*: gi\|2143593\|pir∥S55277 annexi | 2.30 | 19 |
| 424194 | BE245833 | Hs.169854 | gb: TCBAP1E1908 Pediatric pre-B cell acut | 2.30 | 1813 |
| 412661 | N32860 | Hs.24611 | ESTs, Weakly similar to I54374 gene NF2 | 2.30 | 657 |
| 445523 | Z30118 | Hs.293788 | ESTs, Moderately similar to unnamed prot | 2.30 | 3682 |
| 405705 | | | C12000104*: gi\|4503519\|ref\|NP_003745.1\|e | 2.30 | |
| 420596 | NM_002692 | Hs.99185 | polymerase (DNA directed), epsilon 2 | 2.30 | 1435 5082 |
| 429433 | AA452899 | Hs.213586 | ESTs, Weakly similar to KIAA1353 protein | 2.30 | 2368 |
| 455540 | BE080231 | | gb: RC4-BT0629-120200-012-f11 BT0629 Homo | 2.30 | 4514 |
| 426088 | AF038007 | Hs.166196 | ATPase, Class I, type 8B, member 1 | 2.30 | 2024 5289 |
| 414485 | W27026 | Hs.182625 | VAMP (vesicle-associated membrane protei | 2.30 | 835 |
| 426427 | M86699 | Hs.169840 | TTK protein kinase | 2.30 | 2065 5303 |
| 407633 | NM_007069 | Hs.37189 | similar to rat HREV107 | 2.30 | 173 4776 |
| 437659 | AB007944 | Hs.5737 | KIAA0475 gene product | 2.30 | 3079 5589 |
| 438510 | AL080220 | Hs.6285 | DKFZP586P0123 protein | 2.30 | 3143 5595 |
| 414178 | AW957372 | Hs.46791 | ESTs, Weakly similar to I38022 hypotheti | 2.30 | 797 |
| 424339 | BE257148 | | endoglycan | 2.30 | 1831 |
| 419594 | AA013051 | Hs.91417 | topoisomerase (DNA) II binding protein | 2.29 | 1339 |
| 425424 | NM_004954 | Hs.157199 | ELKL motif kinase | 2.29 | 1962 5263 |
| 424649 | BE242035 | Hs.151461 | embryonic ectoderm development | 2.29 | 1867 |
| 408839 | AW277084 | | gb: xp61h09.x1 NCI_CGAP_Ov39 Homo sapiens | 2.29 | 311 |
| 414883 | AA926960 | | CDC28 protein kinase 1 | 2.29 | 887 |
| 453454 | AW052006 | | PRP4/STK/WD splicing factor | 2.29 | 4407 |
| 446080 | AI221741 | Hs.117777 | ESTs | 2.29 | 3723 |
| 450937 | R49131 | Hs.26267 | ATP-dependant interferon response protei | 2.29 | 4177 |
| 459241 | AA032276 | Hs.99010 | ESTs, Moderately similar to T14342 NSD1 | 2.28 | 4661 |
| 426788 | U66615 | Hs.172280 | SWI/SNF related, matrix associated, acti | 2.28 | 2099 5320 |
| 417720 | AA205625 | Hs.208067 | ESTs | 2.28 | 1116 |
| 417542 | J04129 | Hs.82269 | progestagen-associated endometrial prote | 2.28 | 1101 4990 |
| 456141 | AI751357 | Hs.288741 | Homo sapiens cDNA: FLJ22256 fis, clone H | 2.28 | 4529 |
| 425523 | AB007948 | Hs.158244 | KIAA0479 protein | 2.28 | 1973 5268 |
| 403055 | | | C2002219*: gi\|12737280\|ref\|XP_006682.2\|k | 2.28 | |
| 425707 | AF115402 | Hs.11713 | E74-like factor 5 (ets domain transcript | 2.28 | 1992 5277 |
| 418322 | AA284166 | Hs.84113 | cyclin-dependent kinase inhibitor 3 (CDK | 2.28 | 1184 |
| 412505 | AA974491 | Hs.21734 | ESTs | 2.28 | 638 |
| 453863 | X02544 | Hs.572 | orosomucoid 1 | 2.28 | 4438 5786 |
| 410275 | U85658 | Hs.61796 | transcription factor AP-2 gamma (activat | 2.27 | 462 4842 |
| 421535 | AB002359 | Hs.105478 | phosphoribosylformylglycinamidine syntha | 2.27 | 1535 5115 |
| 451150 | AI888124 | Hs.285280 | Homo sapiens cDNA: FLJ22096 fis, clone H | 2.27 | 4197 |
| 423198 | M81933 | Hs.1634 | cell division cycle 25A | 2.27 | 1727 5174 |
| 407777 | AA161071 | Hs.71465 | squalene epoxidase | 2.27 | 194 |
| 451232 | AI769922 | Hs.20023 | ESTs | 2.27 | 4201 |
| 430316 | NM_000875 | Hs.239176 | insulin-like growth factor 1 receptor | 2.27 | 2470 5445 |
| 400884 | | | Target Exon | 2.27 | |
| 436608 | AA628980 | Hs.192371 | down syndrome critical region protein DS | 2.26 | 2997 |
| 434423 | NM_006769 | Hs.3844 | LIM domain only 4 | 2.26 | 2839 5548 |
| 438613 | C05569 | Hs.243122 | hypothetical protein FLJ13057 similar to | 2.26 | 3153 |
| 421180 | BE410992 | Hs.258730 | heme-regulated initiation factor 2-alpha | 2.26 | 1486 |
| 456844 | AI264155 | Hs.152981 | CDP-diacylglycerol synthase (phosphatida | 2.26 | 4562 |
| 422010 | AA302049 | Hs.31181 | Homo sapiens cDNA: FLJ23230 fis, clone C | 2.26 | 1593 |
| 440100 | BE382685 | Hs.158549 | ESTs, Weakly similar to T2D3_HUMAN TRANS | 2.25 | 3263 |
| 445871 | AI702901 | Hs.145582 | ESTs, Weakly similar to FOR4 MOUSE FORMI | 2.25 | 3706 |
| 422955 | AW967824 | No.324237 | ESTs | 2.25 | 1697 |
| 404877 | | | NM_005365: Homo sapiens melanoma antigen, | 2.25 | 4729 63 |
| 401160 | | | Target Exon | 2.25 | |
| 418764 | N30531 | Hs.42215 | protein phosphatase 1, regulatory subuni | 2.25 | 1238 |
| 452243 | AL355715 | Hs.28555 | programmed cell death 9 (PDCD9) | 2.25 | 4287 5756 |
| 452206 | AW340281 | Hs.33074 | Homo sapiens, clone IMAGE: 3606519, mRNA, | 2.25 | 4281 |
| 423290 | AA324130 | Hs.27721 | Wolf-Hirschhorn syndrome candidate 1-lik | 2.25 | 1734 |
| 428405 | Y00762 | Hs.2266 | cholinergic receptor, nicotinic, alpha p | 2.25 | 2250 5357 |
| 413273 | U75679 | Hs.75257 | stem-loop (histone) binding protein | 2.25 | 4896 704 |
| 426028 | NM_001110 | Hs.172028 | a disintegrin and metalloproteinase doma | 2.25 | 2020 5287 |
| 428028 | U52112 | Hs.182018 | interleukin-1 receptor-associated kinase | 2.25 | 2215 |
| 427691 | AW194426 | Hs.20726 | UDP-N-acetyl-alpha-D-galactosamine: polyp | 2.24 | 2181 |
| 429588 | AI080271 | | ESTs | 2.24 | 2391 |
| 429966 | BE081342 | Hs.283037 | HSPC039 protein | 2.24 | 2431 |
| 416655 | AW968613 | Hs.79428 | BCL2/adenovirus E1B 19 kD-interacting pro | 2.24 | 1010 |
| 412452 | AA215731 | | suppression of tumorigenicity 5 | 2.24 | 634 |
| 411580 | AL080088 | Hs.70877 | DKFZP564K2062 protein | 2.24 | 4865 561 |
| 457653 | AI820719 | Hs.76853 | DnaJ (Hsp40) homolog, subfamily A, membe | 2.24 | 4597 |
| 433002 | AF048730 | Hs.279906 | cyclin T1 | 2.24 | 2720 5522 |
| 419362 | N64116 | Hs.24624 | hypothetical protein FLJ21945 | 2.24 | 1306 |
| 434340 | AI193043 | Hs.128685 | ESTs, Weakly similar to T17226 hypotheti | 2.24 | 2830 |
| 420186 | NM_015925 | Hs.95697 | liver-specific bHLH-Zip transcription fa | 2.24 | 1392 5066 |
| 430375 | AW371048 | Hs.93758 | H4 histone family, member H | 2.23 | 2477 |
| 448356 | AL120837 | Hs.20993 | high-glucose-regulated protein 8 | 2.23 | 3958 |
| 420554 | AL133022 | Hs.98845 | Homo sapiens mRNA; cDNA DKFZp434I0121 (f | 2.23 | 1431 |
| 430016 | NM_004736 | Hs.227656 | xenotropic and polytropic retrovirus rec | 2.23 | 2437 5432 |
| 418004 | U37519 | Hs.87539 | aldehyde dehydrogenase 3 family, member | 2.23 | 1145 4997 |
| 440457 | BE387593 | Hs.21321 | Homo sapiens clone FLB9213 PRO2474 mRNA, | 2.22 | 3289 |
| 439924 | AI985897 | Hs.125293 | ESTs | 2.22 | 3242 |
| 415889 | R24563 | | VPS10 domain receptor protein | 2.22 | 957 |
| 421353 | AW292857 | Hs.255130 | ESTs | 2.22 | 1511 |
| 413476 | U25849 | Hs.75393 | acid phosphatase 1, soluble | 2.22 | 4902 726 |
| 430355 | NM_006219 | Hs.239818 | phosphoinositide-3-kinase, catalytic, be | 2.22 | 2475 5449 |
| 442092 | AW578669 | | hypothetical protein FLJ12439 | 2.22 | 3397 |
| 426181 | AA371422 | Hs.334371 | hypothetical protein MGC13096 | 2.22 | 2037 |
| 450074 | AI367213 | Hs.14070 | hypothetical protein FLJ14166 | 2.22 | 4103 |
| 407930 | AA045847 | Hs.188361 | Homo sapiens cDNA FLJ12807 fis, clone NT | 2.22 | 215 |
| 424534 | D87682 | Hs.150275 | KIAA0241 protein | 2.21 | 1856 5215 |
| 432195 | AJ243669 | Hs.8127 | KIAA0144 gene product | 2.21 | 2642 5503 |
| 420734 | AW972872 | Hs.293736 | ESTs | 2.21 | 1447 |
| 422390 | AW450893 | Hs.121830 | ESTs, Weakly similar to T42682 hypotheti | 2.21 | 1638 |
| 414761 | AU077228 | Hs.77256 | enhancer of zeste (Drosophila) homolog 2 | 2.21 | 875 |
| 414915 | NM_002462 | Hs.76391 | myxovirus (influenza) resistance 1, homo | 2.21 | 4934 892 |
| 418030 | BE207573 | Hs.83321 | neuromedin B | 2.21 | 1150 |
| 424736 | AF230877 | Hs.152701 | microtubule-interacting protein that ass | 2.21 | 1880 5230 |
| 428788 | AF082283 | Hs.193516 | B-cell CLL/lymphoma 10 | 2.21 | 2299 5378 |
| 408681 | AW953853 | Hs.281462 | ESTs, Weakly similar to I38022 hypotheti | 2.21 | 293 |
| 419485 | AA489023 | Hs.99807 | ESTs, Weakly similar to unnamed protein | 2.21 | 1318 |
| 446627 | AI973016 | Hs.15725 | hypothetical protein SBBI48 | 2.20 | 3772 |
| 440704 | M69241 | Hs.162 | insulin-like growth factor binding prote | 2.20 | 3314 5613 |
| 436905 | N31273 | Hs.42380 | ESTs | 2.20 | 3020 |
| 454034 | NM_000691 | Hs.575 | aldehyde dehydrogenase 3 family, member | 2.20 | 4462 5792 |
| 459376 | BE258770 | | Homo sapiens, clone IMAGE: 3344506, mRNA, | 2.20 | 4667 |
| 444527 | NM_005408 | Hs.11383 | small inducible cytokine subfamily A (Cy | 2.20 | 3605 5640 |
| 403429 | | | C3000329*: gi\|8922921\|ref\|NP_060821.1\| hy | 2.20 | |
| 416814 | AW192307 | Hs.80042 | dolichyl-P-Glc: Man9GlcNAc2-PP-dolichylgl | 2.20 | 1026 |
| 403154 | | | NM_022780*: Homo sapiens hypothetical pro | 2.20 | 43 4712 |
| 410434 | AF051152 | Hs.63668 | toll-like receptor 2 | 2.20 | 478 4847 |
| 425810 | AI923627 | Hs.31903 | ESTs | 2.20 | 1998 |
| 417173 | U61397 | Hs.81424 | ubiquitin-like 1 (sentrin) | 2.20 | 1068 4979 |
| 437587 | AI591222 | Hs.72325 | Human DNA sequence from clone RP1-187J11 | 2.20 | 3072 |
| 423020 | AA383092 | Hs.1608 | replication protein A3 (14 kD) | 2.20 | 1711 |
| 429626 | U36787 | Hs.211571 | holocytochrome c synthase (cytochrome c | 2.20 | 2399 5418 |
| 417933 | X02308 | Hs.82962 | thymidylate synthetase | 2.20 | 1139 4996 |
| 436972 | AA284679 | Hs.25640 | claudin 3 | 2.20 | 3024 |
| 421977 | W94197 | Hs.110165 | ribosomal protein L26 homolog | 2.20 | 1588 |
| 416658 | U03272 | Hs.79432 | fibrillin 2 (congenital contractural ara | 2.20 | 1011 4966 |
| 417750 | AI267720 | Hs.260523 | synovial sarcoma, translocated to X chro | 2.19 | 1120 |
| 413281 | AA861271 | Hs.222024 | transcription factor BMAL2 | 2.19 | 706 |
| 408150 | BE620274 | Hs.43112 | Homo sapiens mRNA; cDNA DKFZp434B1620 (f | 2.19 | 238 |
| 422134 | AW179019 | Hs.112110 | mitochondrial ribosomal protein L42 | 2.19 | 1608 |
| 410723 | AA100683 | | basigin (OK blood group) | 2.19 | 511 |
| 445786 | AW629819 | Hs.144502 | hypothetical protein FLJ22055 | 2.19 | 3697 |
| 449162 | AI632740 | Hs.10476 | ESTs | 2.19 | 4041 |
| 416097 | BE387371 | Hs.118964 | hypothetical protein FLJ20085 | 2.19 | 971 |
| 429343 | AK000785 | Hs.199480 | Homo sapiens, Similar to epsin 3, clone | 2.18 | 2356 5402 |
| 409456 | U34962 | Hs.54473 | cardiac-specific homeo box | 2.18 | 374 4825 |
| 411734 | AW374954 | Hs.71779 | Homo sapiens DNA from chromosome 19, cos | 2.18 | 572 |
| 401833 | | | integrin-linked kinase-associated serine | 2.18 | |
| 409139 | AI681917 | Hs.3321 | ESTs, Highly similar to IRX1_HUMAN IROQU | 2.18 | 339 |
| 446567 | NM_007247 | Hs.15384 | AP1 gamma subunit binding protein 1 | 2.18 | 3764 5672 |
| 440334 | BE276112 | Hs.7165 | zinc finger protein 259 | 2.17 | 3283 |
| 406627 | T64904 | Hs.163780 | ESTs | 2.17 | 80 |
| 447842 | AW160804 | Hs.247302 | twisted gastrulation | 2.17 | 3913 |
| 417433 | BE270266 | Hs.82128 | 5T4 oncofetal trophoblast glycoprotein | 2.17 | 1094 |
| 428781 | AF164799 | Hs.193384 | putatative 28 kDa protein | 2.17 | 2297 5376 |
| 410512 | AA085603 | Hs.250570 | hypothetical protein MGC3180 | 2.17 | 487 |
| 436995 | AI160015 | Hs.125489 | ESTs | 2.17 | 3026 |
| 442577 | AA292998 | Hs.163900 | ESTs | 2.17 | 3447 |
| 402322 | | | Target Exon | 2.17 | |
| 429462 | AI890356 | | Homo sapiens, clone IMAGE: 3536432, mRNA, | 2.17 | 2373 |
| 412326 | R07566 | Hs.73817 | small inducible cytokine A3 (homologous | 2.17 | 624 |
| 426722 | U53823 | Hs.171952 | occludin | 2.17 | 2091 5316 |
| 406867 | AA157857 | Hs.182265 | keratin 19 | 2.17 | 105 |
| 424399 | AI905687 | | AI905687: IL-BT095-190199-019 BT095 Homo | 2.17 | 1840 |
| 427709 | AI631811 | Hs.180403 | STRIN protein | 2.16 | 2186 |
| 410627 | AA181339 | Hs.929 | myosin, heavy polypeptide 7, cardiac mus | 2.16 | 499 |
| 429901 | AK000502 | Hs.56237 | hypothetical protein FLJ20495 | 2.16 | 2424 5429 |
| 440282 | BE262386 | | clones 23667 and 23775 zinc finger prote | 2.16 | 3277 |
| 422975 | AA347720 | Hs.122669 | KIAA0264 protein | 2.16 | 1704 |
| 427254 | AL121523 | Hs.97774 | ESTs | 2.16 | 2135 |
| 444006 | BE395085 | Hs.10086 | type I transmembrane protein Fn14 | 2.16 | 3568 |
| 424921 | AA348491 | Hs.322456 | hypothetical protein DKFZp761D0211 | 2.16 | 1899 |
| 405204 | | | NM_002086*: Homo sapiens growth factor re | 2.16 | 4731 65 |
| 412988 | BE046680 | | gb: hn42h03.x1 NCI_CGAP_RDF2 Homo sapiens | 2.15 | |
| 444151 | AW972917 | Hs.128749 | alpha-methylacyl-CoA racemase | 2.15 | 3577 |
| 417185 | NM_002484 | Hs.81469 | nucleotide binding protein 1 (E. coliMin | 2.15 | 1070 4980 |
| 447229 | BE617135 | Hs.22612 | hypothetical protein DKFZp566D1346 | 2.15 | 3832 |
| 414727 | BE466904 | Hs.190162 | gb: hz28f03.x1 NCI_CGAP_GC6 Homo sapiens | 2.15 | 870 |
| 435542 | AA687376 | | ESTs | 2.15 | 2925 |
| 410307 | AF022913 | Hs.62187 | phosphatidylinositol glycan, class K | 2.15 | 464 4844 |
| 442611 | BE077155 | Hs.177537 | hypothetical protein DKFZp761B1514 | 2.15 | 3453 |
| 418849 | AW474547 | Hs.53565 | Homo sapiens PIG-M mRNA for mannosyltran | 2.15 | 1249 |
| 418647 | AA226198 | | gb: nc26a07.s1 NCI_CGAP_Pr1 Homo sapiens | 2.15 | 1218 |
| 422330 | D30783 | Hs.115263 | epiregulin | 2.15 | 1632 5147 |
| 418437 | AA771738 | Hs.348000 | ESTs, Moderately similar to ALU5_HUMAN A | 2.15 | 1201 |
| 457100 | AA417878 | Hs.48401 | ESTs, Moderately similar to ALU8_HUMAN A | 2.15 | 4578 |
| 407748 | AL079409 | Hs.38176 | KIAA0606 protein; SCN Circadian Oscillat | 2.15 | 188 |
| 444070 | NM_015367 | Hs.10267 | MIL1 protein | 2.15 | 3571 5635 |
| 408901 | AK001330 | Hs.48855 | hypothetical protein FLJ10468 | 2.15 | 316 4808 |
| 444927 | AW016637 | Hs.199425 | ESTs | 2.15 | 3640 |
| 429986 | AF092047 | Hs.227277 | sine oculis homeobox (Drosophila) homolo | 2.15 | 2434 5431 |
| 430178 | AW449612 | Hs.152475 | 3′UTR of: achaete-scute complex (Drosoph | 2.15 | 2451 |
| 410252 | AW821182 | Hs.61418 | microfibrillar-associated protein 1 | 2.15 | 460 |
| 447669 | AL049985 | Hs.19180 | Homo sapiens mRNA; cDNA DKFZp564E122 (fr | 2.15 | 3888 |
| 437270 | R18087 | Hs.323769 | cisplatin resistance related protein CRR | 2.14 | 3051 |
| 458809 | AW972512 | Hs.20985 | sin3-associated polypeptide, 30 kD | 2.14 | 4637 |
| 418057 | NM_012151 | Hs.83363 | coagulation factor VIII-associated (intr | 2.14 | 1153 5002 |
| 415279 | F04237 | Hs.1447 | glial fibrillary acidic protein | 2.14 | 923 |
| 444418 | AL034417 | Hs.11169 | Gene 33/Mig-6 | 2.14 | 3596 |
| 430301 | AI902657 | Hs.188662 | KIAA1702 protein | 2.14 | 2466 |
| 447587 | AW292139 | Hs.115789 | ESTs | 2.14 | 3884 |
| 442767 | AI017208 | Hs.131149 | ESTs | 2.14 | 3467 |
| 425811 | AL039104 | Hs.159557 | karyopherin alpha 2 (RAG cohort 1, impor | 2.14 | 1999 |
| 458882 | R34993 | Hs.226666 | ESTs, Moderately similar to I54374 gene | 2.14 | 4643 |
| 436854 | AA749167 | Hs.173911 | ESTs | 2.13 | 3010 |
| 439588 | AA838166 | Hs.174644 | hypothetical protein FLJ21669 | 2.13 | 3221 |
| 416975 | NM_004131 | Hs.1051 | granzyme B (granzyme 2, cytotoxic T-lymp | 2.13 | 1045 4974 |
| 427715 | BE245274 | Hs.180428 | KIAA1181 protein | 2.13 | 2188 |
| 415014 | AW954064 | Hs.24951 | ESTs | 2.13 | 900 |
| 417389 | BE260964 | Hs.82045 | midkine (neurite growth-promoting factor | 2.13 | 1091 |
| 449955 | AI676010 | Hs.224043 | ESTs | 2.13 | 4093 |
| 448920 | AW408009 | Hs.22580 | alkylglycerone phosphate synthase | 2.13 | 4020 |
| 419767 | W73306 | Hs.306668 | Homo sapiens cDNA FLJ14089 fis, clone MA | 2.13 | 1361 |
| 427674 | NM_003528 | Hs.2178 | H2B histone family, member Q | 2.13 | 2177 5342 |
| 424765 | AA428211 | | hypothetical protein FLJ14033 similar to | 2.13 | 1883 |
| 439306 | BE220199 | | WD40 protein Ciao1 | 2.13 | 3197 |
| 416361 | AW204907 | Hs.6872 | ESTs, Weakly similar to CA13_HUMAN COLLA | 2.12 | 989 |
| 407792 | AI077715 | Hs.39384 | putative secreted ligand homologous to f | 2.12 | 196 |
| 421875 | AA299607 | Hs.98969 | ESTs | 2.12 | 1574 |
| 422405 | AA310278 | | ESTs | 2.12 | 1640 |
| 414907 | X90725 | Hs.77597 | polo (Drosophia)-like kinase | 2.12 | 4933 891 |
| 414602 | AW630088 | Hs.76550 | Homo sapiens mRNA; cDNA DKFZp564B1264 (f | 2.12 | 853 |
| 400203 | | | Eos Control | 2.12 | |
| 427779 | AA906997 | Hs.180780 | TERA protein | 2.11 | 2195 |
| 419667 | AU077005 | Hs.92208 | a disintegrin and metalloproteinase doma | 2.11 | 1346 |
| 420460 | AA262331 | Hs.48376 | Homo sapiens clone HB-2 mRNA sequence | 2.11 | 1423 |
| 447711 | AI459554 | Hs.161286 | ESTs | 2.11 | 3895 |
| 413838 | AV661185 | Hs.75574 | mitochondrial ribosomal protein L19 | 2.11 | 757 |
| 419373 | NM_003244 | Hs.90077 | TG-interacting factor (TALE family homeo | 2.11 | 1307 5045 |
| 419741 | NM_007019 | Hs.93002 | ubiquitin carrier protein E2-C | 2.11 | 1355 5056 |
| 452429 | AK000149 | Hs.29493 | hypothetical protein FLJ20142 | 2.11 | 4307 5758 |
| 431797 | BE169641 | Hs.270134 | hypothetical protein FLJ20280 | 2.11 | 2601 |
| 421690 | AW162667 | Hs.106857 | calbindin 2, (29 kD, calretinin) | 2.11 | 1554 |
| 446994 | AV650435 | Hs.16755 | MBIP protein | 2.11 | 3809 |
| 424629 | M90656 | Hs.151393 | glutamate-cysteine ligase, catalytic sub | 2.10 | 1865 5221 |
| 426216 | N77630 | Hs.13895 | Homo sapiens cDNA FLJ11654 fis, clone HE | 2.10 | 2040 |
| 403961 | | | Target Exon | 2.10 | |
| 434170 | AA626509 | Hs.122329 | ESTs | 2.10 | 2817 |
| 432734 | AA837396 | Hs.263925 | LIS1-interacting protein NUDE1, rat homo | 2.10 | 2696 |
| 414998 | NM_002543 | Hs.77729 | oxidised low density lipoprotein (lectin | 2.10 | 4938 899 |
| 426410 | BE298446 | Hs.305890 | BCL2-like 1 | 2.10 | 2063 |
| 412700 | BE222433 | | ESTs, Weakly similar to I38022 hypotheti | 2.10 | 660 |
| 418397 | NM_001269 | Hs.84746 | chromosome condensation 1 | 2.10 | 1195 5013 |
| 449249 | T52285 | Hs.193115 | Homo sapiens mRNA for KIAA1764 protein, | 2.10 | 4050 |
| 447401 | BE618582 | Hs.97661 | ESTs | 2.10 | 3858 |
| 433745 | AF075320 | Hs.28980 | hypothetical protein FLJ14540 | 2.10 | 2786 |
| 410232 | AW372451 | Hs.61184 | CGI-79 protein | 2.10 | 458 |
| 452335 | AW188944 | Hs.61272 | ESTs | 2.10 | 4297 |
| 454427 | AW605620 | Hs.76064 | ribosomal protein L27a | 2.10 | 4483 |
| 406740 | AA577274 | | gb: nm85g07.s1 NCI_CGAP_Co9 Homo sapiens | 2.10 | 95 |
| 403969 | | | ENSP00000034663: Zinc finger protein 131 | 2.10 | |
| 408405 | AK001332 | Hs.44672 | hypothetical protein FLJ10470 | 2.10 | 264 4796 |
| 433160 | AW207002 | Hs.134342 | TASP for testis-specific adriamycin sens | 2.09 | 2742 |
| 425118 | AU076611 | Hs.154672 | methylene tetrahydrofolate dehydrogenase | 2.09 | 1923 |
| 448556 | AW885606 | Hs.5064 | ESTs | 2.09 | 3985 |
| 428966 | AF059214 | Hs.194687 | cholesterol 25-hydroxylase | 2.09 | 2320 5385 |
| 447887 | AA114050 | Hs.19949 | caspase 8, apoptosis-related cysteine pr | 2.09 | 3918 |
| 426820 | U73328 | Hs.172648 | distal-less homeobox 4 | 2.09 | 2102 5322 |
| 435538 | AB011540 | Hs.4930 | low density lipoprotein receptor-related | 2.09 | 2924 5565 |
| 449008 | AW578003 | Hs.22826 | tropomodulin 3 (ubiquitous) | 2.09 | 4026 |
| 419252 | AW138434 | Hs.129805 | ESTs | 2.08 | 1293 |
| 401192 | | | Target Exon | 2.08 | |
| 419152 | L12711 | Hs.89643 | transketolase (Wernicke-Korsakoff syndro | 2.08 | 1283 5040 |
| 453164 | F33692 | Hs.32018 | SNARE associated protein snapin | 2.08 | 4381 |
| 426158 | NM_001982 | Hs.199067 | v-erb-b2 avian erythroblastic leukemia v | 2.08 | 2033 5294 |
| 422616 | BE300330 | Hs.118725 | selenophosphate synthetase 2 | 2.08 | 1663 |
| 422684 | BE561617 | Hs.119192 | H2A histone family, member Z | 2.08 | 1673 |
| 431630 | NM_002204 | Hs.265829 | integrin, alpha 3 (antigen CD49C, alpha | 2.08 | 2578 5483 |
| 414341 | D80004 | Hs.75909 | KIAA0182 protein | 2.08 | 4919 813 |
| 456806 | AI222298 | Hs.140720 | GSK-3 binding protein FRAT2 | 2.08 | 4561 |
| 459646 | AW883968 | Hs.321190 | gb: QV3-OT0063-290300-135-c04 OT0063 Homo | 2.08 | 4674 |
| 454417 | AI244459 | Hs.110826 | trinucleotide repeat containing 9 | 2.08 | 4480 |
| 442481 | N99828 | | gb: za32c04.r1 Soares fetal liver spleen | 2.07 | 3434 |
| 430677 | Z26317 | | desmoglein 2 | 2.07 | 2504 5461 |
| 420319 | AW406289 | Hs.96593 | hypothetical protein | 2.07 | 1410 |
| 448633 | AA311426 | Hs.21635 | tubulin, gamma 1 | 2.07 | 3990 |
| 417863 | AB000450 | Hs.82771 | vaccinia related kinase 2 | 2.07 | 1131 4995 |
| 411263 | BE297802 | Hs.69360 | kinesin-like 6 (mitotic centromere-assoc | 2.07 | 544 |
| 450157 | AW961576 | Hs.60178 | ESTs | 2.07 | 4107 |
| 410491 | AA465131 | Hs.64001 | Homo sapiens clone 25218 mRNA sequence | 2.07 | 484 |
| 440594 | AW445167 | Hs.126036 | ESTs | 2.07 | 3302 |
| 401832 | | | integrin-linked kinase-associated serine | 2.06 | |
| 401797 | | | Target Exon | 2.06 | |
| 443683 | BE241717 | Hs.9676 | uncharacterized hypothalamus protein HT0 | 2.06 | 3539 |
| 425474 | Z48054 | Hs.158084 | peroxisome receptor 1 | 2.06 | 1970 |
| 433658 | L03678 | Hs.156110 | immunoglobulin kappa constant | 2.06 | 2778 5539 |
| 452012 | AA307703 | Hs.279766 | kinesin family member 4A | 2.06 | 4262 |
| 453331 | AI240665 | | ESTs | 2.06 | 4396 |
| 410407 | X66839 | Hs.63287 | carbonic anhydrase IX | 2.06 | 474 4846 |
| 414983 | L17128 | Hs.77719 | gamma-glutamyl carboxylase | 2.06 | 4937 897 |
| 456465 | M94065 | Hs.94925 | dihydroorotate dehydrogenase | 2.06 | 4544 5798 |
| 446111 | W56338 | Hs.13880 | CGI-143 protein | 2.06 | 3725 |
| 409190 | AU076536 | Hs.50984 | sarcoma amplified sequence | 2.06 | 349 |
| 446163 | AA026880 | Hs.25252 | prolactin receptor | 2.06 | 3731 |
| 404029 | | | NM_018936*: Homo sapiens protocadherin be | 2.06 | 4718 50 |
| 437108 | AA434054 | Hs.80624 | hypothetical protein MGC2560 | 2.06 | 3034 |
| 452110 | T47667 | Hs.28005 | Homo sapiens cDNA FLJ11309 fis, clone PL | 2.05 | 4272 |
| 428753 | AW939252 | Hs.192927 | hypothetical protein FLJ20251 | 2.05 | 2291 |
| 427752 | AA470687 | Hs.104772 | ESTs | 2.05 | 2194 |
| 410976 | R36207 | Hs.25092 | hypothetical protein MGC10744 | 2.05 | 525 |
| 409960 | BE261944 | | hexokinase 1 | 2.05 | 422 |
| 452721 | AJ269529 | Hs.301871 | solute carrier family 37 (glycerol-3-pho | 2.05 | 4337 5761 |
| 446269 | AW263155 | Hs.14559 | hypothetical protein FLJ10540 | 2.05 | 3741 |
| 411761 | AI733848 | Hs.71935 | putative zinc finger protein from EUROIM | 2.05 | 574 |
| 420942 | H03514 | Hs.15589 | ESTs | 2.05 | 1467 |
| 408673 | BE208517 | | ribosomal protein L37a | 2.05 | 292 |
| 406709 | AI355761 | Hs.242463 | keratin 8 | 2.05 | 91 |
| 447128 | AI271898 | | cyclin K | 2.05 | 3825 |
| 449437 | AI702038 | Hs.100057 | Homo sapiens cDNA: FLJ22902 fis, clone K | 2.05 | 4061 |
| 416812 | H91010 | Hs.44940 | ESTs | 2.05 | 1025 |
| 404831 | | | C1002937*: gi\|7499208\|pir\|\|T20993 hypothe | 2.05 | |
| 408841 | AW438865 | Hs.256862 | ESTs | 2.05 | 312 |
| 410656 | BE161335 | Hs.321717 | ESTs, Weakly similar to S22765 heterogen | 2.05 | 501 |
| 418319 | AW611703 | Hs.190173 | ESTs, Weakly similar to A46010 X-linked | 2.05 | 1183 |
| 400249 | | | Eos Control | 2.05 | |
| 439092 | AA830149 | | gb: oc44f08.s1 NCI_CGAP_GCB1 Homo sapiens | 2.05 | 3185 |
| 448390 | AL035414 | Hs.21068 | hypothetical protein | 2.05 | 3963 |
| 440953 | AI683036 | Hs.124135 | Homo sapiens cDNA FLJ13051 fis, clone NT | 2.05 | 3321 |
| 427178 | AA398866 | Hs.97542 | Homo sapiens testis-development related | 2.05 | 2130 |
| 401463 | | | histone deacetylase 5 | 2.05 | |
| 453849 | N35321 | Hs.278611 | UDP-N-acetyl-alpha-D-galactosamine: polyp | 2.05 | 4436 |
| 426172 | AA371307 | Hs.125056 | ESTs | 2.05 | 2035 |
| 423871 | AA331906 | Hs.175596 | gb: EST35805 Embryo, 8 week I Homo sapien | 2.05 | 1783 |
| 418690 | AK000052 | Hs.87293 | hypothetical protein FLJ20045 | 2.05 | 1228 5026 |
| 423248 | AA380177 | Hs.125845 | ribulose-5-phosphate-3-epimerase | 2.05 | 1731 |
| 427356 | AW023482 | Hs.97849 | ESTs | 2.05 | 2147 |
| 435147 | AL133731 | Hs.4774 | Homo sapiens mRNA; cDNA DKFZp761C1712 (f | 2.05 | 2900 |
| 415025 | AW207091 | Hs.72307 | ESTs | 2.05 | 902 |
| 437763 | AA469369 | Hs.5831 | tissue inhibitor of metalloproteinase 1 | 2.05 | 3089 |
| 413916 | N49813 | Hs.75615 | apolipoprotein C-II | 2.05 | 763 |
| 452177 | AI863447 | Hs.268180 | gb: tz48f01.x1 NCI_CGAP_Brn52 Homo sapien | 2.05 | 4279 |
| 407590 | AI831258 | | ESTs | 2.05 | 171 |
| 431250 | BE264649 | Hs.251377 | taxol resistance associated gene 3 | 2.05 | 2544 |
| 430750 | AI650360 | Hs.100256 | ESTs | 2.05 | 2511 |
| 448296 | BE622756 | Hs.10949 | Homo sapiens cDNA FLJ14162 fis, clone NT | 2.05 | 3956 |
| 447211 | AL161961 | Hs.17767 | KIAA1554 protein | 2.05 | 3830 |
| 412939 | AW411491 | Hs.75069 | eukaryotic translation elongation factor | 2.05 | 684 |
| 420309 | AW043637 | Hs.21766 | ESTs, Weakly similar to ALU5_HUMAN ALU S | 2.04 | 1408 |
| 422321 | AA906427 | Hs.181035 | hypothetical protein MGC11296 | 2.04 | 1631 |
| 424287 | AL133105 | Hs.144633 | hypothetical protein DKFZp434F2322 | 2.04 | 1825 5208 |
| 432878 | BE386490 | Hs.279663 | Pirin | 2.04 | 2707 |
| 408113 | T82427 | Hs.194101 | Homo sapiens cDNA: FLJ20869 fis, clone A | 2.04 | 235 |
| 447973 | AB011169 | Hs.20141 | similar to S. cerevisiaeSSM4 | 2.04 | 3929 5704 |
| 410553 | AW016824 | Hs.272068 | hypothetical protein MGC14128 | 2.04 | 491 |
| 416990 | AF124145 | Hs.80731 | autocrine motility factor receptor | 2.04 | 1049 4975 |
| 451273 | NM_014811 | Hs.26163 | KIAA0649 gene product | 2.04 | 4206 5741 |
| 412507 | L36645 | Hs.73964 | EphA4 | 2.04 | 4880 639 |
| 433257 | AA613437 | Hs.302743 | hypothetical protein FLJ12543 | 2.04 | 2751 |
| 443823 | BE089782 | Hs.9877 | hypothetical protein | 2.04 | 3555 |
| 403532 | | | NM_024638: Homo sapiens hypothetical prot | 2.04 | 46 4715 |
| 437159 | AL050072 | Hs.306313 | Homo sapiens mRNA; cDNA DKFZp566E1346 (f | 2.03 | 3040 |
| 407813 | AL120247 | Hs.40109 | KIAA0872 protein | 2.03 | 200 |
| 440773 | AA352702 | Hs.37747 | Homo sapiens, Similar to RIKEN cDNA 2700 | 2.03 | 3318 |
| 424036 | AA770688 | | H2A histone family, member L | 2.03 | 1793 |
| 409463 | AI458165 | Hs.17296 | hypothetical protein MGC2376 | 2.03 | 375 |
| 445929 | AI089660 | Hs.323401 | dpy-30-like protein | 2.03 | 3714 |
| 452046 | AB018345 | Hs.27657 | KIAA0802 protein | 2.03 | 4266 5754 |
| 443347 | AI052543 | Hs.133244 | melanoma-derived leucine zipper, extra-n | 2.03 | 3519 |
| 405264 | | | NM_030813*: Homo sapiens suppressor of po | 2.03 | 4732 66 |
| 408756 | AA524743 | | ESTs | 2.03 | 303 |
| 417308 | H60720 | Hs.81892 | KIAA0101 gene product | 2.03 | 1079 |
| 416391 | AI878927 | Hs.79284 | mesoderm specific transcript (mouse) hom | 2.03 | 992 |
| 453055 | AW291436 | Hs.31917 | Homo sapiens, clone MGC: 9658, mRNA, comp | 2.03 | 4370 |
| 427484 | N32859 | Hs.37288 | nuclear receptor subfamily 1, group D, m | 2.03 | 2155 |
| 419852 | AW503756 | Hs.286184 | hypothetical protein dJ551D2.5 | 2.03 | 1363 |
| 432673 | AB028859 | Hs.278605 | DnaJ (Hsp40) homolog, subfamily B, membe | 2.03 | 2689 5510 |
| 424381 | AA285249 | Hs.146329 | protein kinase Chk2 (CHEK2) | 2.02 | 1838 |
| 443826 | AI214805 | Hs.27232 | ESTs | 2.02 | 3556 |
| 456258 | AW976410 | Hs.289069 | Homo sapiens clone FLB3411 PRO0852 mRNA, | 2.02 | 4535 |
| 418105 | AW937488 | Hs.246381 | ESTs, Weakly similar to FV1 MOUSE FRIEND | 2.02 | 1160 |
| 409445 | AW341217 | Hs.14139 | ESTs, Weakly similar to JC5314 CDC28/cdc | 2.02 | 373 |
| 437016 | AU076916 | Hs.5398 | guanine monphosphate synthetase | 2.02 | 3027 |
| 449230 | BE613348 | | melanoma cell adhesion molecule | 2.02 | 4049 |
| 424527 | AW138558 | Hs.334873 | ESTs, Weakly similar to I54374 gene NF2 | 2.02 | 1855 |
| 419239 | AA468183 | Hs.184598 | Homo sapiens cDNA: FLJ23241 fis, clone C | 2.02 | 1292 |
| 427982 | NM_016156 | Hs.181326 | KIAA1073 protein | 2.02 | 2210 5349 |
| 410855 | X97795 | Hs.66718 | RAD54 (S. cerevisiae)-like | 2.02 | 4858 518 |
| 437897 | AA770561 | Hs.146170 | hypothetical protein FLJ22969 | 2.01 | 3104 |
| 449000 | U69560 | Hs.3826 | kelch-like protein C3IP1 | 2.01 | 4025 |
| 437435 | AA249439 | Hs.27027 | hypothetical protein DKFZp762H1311 | 2.01 | 3065 |
| 421757 | Z20897 | Hs.296259 | paraoxonase 3 | 2.01 | 1559 |
| 436238 | AK002163 | Hs.301724 | hypothetical protein FLJ11301 | 2.01 | 2969 5577 |
| 436856 | AI469355 | Hs.127310 | ESTs | 2.01 | 3011 |
| 432026 | AA524545 | Hs.224630 | ESTs | 2.01 | 2627 |
| 407230 | AA157857 | Hs.182265 | keratin 19 | 2.01 | 139 |
| 447701 | BE619526 | Hs.272068 | hypothetical protein MGC14128 | 2.01 | 3893 |
| 418583 | AA604379 | Hs.86211 | hypothetical protein | 2.01 | 1214 |
| 408946 | AW854991 | Hs.255565 | ESTs | 2.01 | 318 |
| 419440 | AB020689 | Hs.90419 | KIAA0882 protein | 2.01 | 1313 5047 |
| 450983 | AA305384 | Hs.25740 | ERO1 (S. cerevisiae)-like | 2.01 | 4180 |
| 414825 | X06370 | Hs.77432 | epidermal growth factor receptor (avian | 2.01 | 4930 882 |
| 410701 | AF198620 | Hs.10283 | RNA binding motif protein 8A | 2.01 | 4854 508 |
| 407317 | AI204033 | Hs.30792 | ESTs, Weakly similar to I38022 hypotheti | 2.01 | 151 |
| 423551 | AA327598 | Hs.89633 | ESTs | 2.01 | 1757 |
| 400810 | | | NM_006560: Homo sapiens CUG triplet repea | 2.00 | 20 4691 |
| 407896 | D76435 | Hs.41154 | Zic family member 1 (odd-paired Drosophi | 2.00 | 213 4785 |
| 413129 | AF292100 | Hs.104613 | RP42 homolog | 2.00 | 4893 693 |
| 427871 | AW992405 | Hs.59622 | Homo sapiens, clone IMAGE: 3507281, mRNA, | 2.00 | 2202 |
| 444042 | NM_004915 | Hs.10237 | ATP-binding cassette, sub-family G (WHIT | 2.00 | 3570 5634 |
| 446770 | AV660309 | Hs.154986 | ESTs, Weakly similar to PLLP_HUMAN PLASM | 2.00 | 3782 |
| 414390 | BE281040 | | gb: 601156234F1 NIH_MGC_21 Homo sapiens c | 2.00 | 820 |
| 400074 | | | Eos Control | 2.00 | |
| 451684 | AF216751 | Hs.26813 | CDA14 | 2.00 | 4240 5747 |
| 451106 | BE382701 | Hs.25960 | N-MYC oncogene | 2.00 | 4193 |
| 419465 | AW500239 | Hs.21187 | Homo sapiens cDNA: FLJ23068 fis, clone L | 2.00 | 1316 |
| 409988 | N27687 | Hs.334334 | transcription factor AP-2 alpha (activat | 2.00 | 426 |
| 458422 | AI344782 | | DnaJ (Hsp40) homolog, subfamily C, membe | 2.00 | 4624 |
| 434636 | AA083764 | | hypothetical protein MGC3178 | 2.00 | 2856 |
| 442007 | AA301116 | Hs.142838 | nucleolar phosphoprotein Nopp34 | 2.00 | 3387 |
| 414569 | AF109298 | Hs.118258 | prostate cancer associated protein 1 | 2.00 | 845 |
| 441991 | AW844404 | Hs.126901 | Homo sapiens mRNA full length insert cDN | 2.00 | 3386 |
| 410025 | BE220489 | Hs.113592 | ESTs, Moderately similar to I54374 gene | 2.00 | 432 |
| 436961 | AW375974 | Hs.156704 | ESTs | 2.00 | 3023 |
| 411007 | AA311529 | Hs.67619 | hypothetical protein My014 | 2.00 | 527 |
| 421808 | AK000157 | Hs.108502 | hypothetical protein FLJ20150 | 2.00 | 1565 5122 |
| 450341 | N90956 | Hs.17230 | hypothetical protein FLJ22087 | 2.00 | 4131 |
| 446565 | D13757 | Hs.311 | phosphoribosyl pyrophosphate amidotransf | 2.00 | 3763 5671 |
| 449636 | AI656608 | Hs.281328 | ESTs, Weakly similar to T00378 KIAA0641 | 2.00 | 4071 |

TABLE 11B

| Pkey | CAT number | Accession |
|---|---|---|
| 429228 | 215430_1 | BG676155 BM009591 AI479075 AI025794 AI017967 AA448270 BE466812 AA853422 AI392649 BG952034 AA513384 BF840124 BE714620 |
| | | AW969605 AI553633 |
| 451807 | 17758_2 | BM479185 AL552795 AL577722 BF038888 BM127617 BF510346 AW450652 AA865478 AW449519 BM127314 AI806539 AW449522 |
| | | AA993634 AI827626 AA904788 |
| 421798 | 3042_4 | BC017829 AW276646 AI984209 AA663933 AA634104 AA551528 AA634041 AA298038 BG483990 T89297 BF853958 H64685 T90329 |
| | | T60644 T57747 BF852694 T92529 BG482852 BF883064 BF883066 N74880 AA829796 N90716 |
| 428342 | 6712_1 | AK056315 AI015524 AA724079 BI713619 AI377728 AW293682 AI928140 AI092404 AI085630 AA731340 BM469629 AW968804 AA425658 |
| | | AA769094 BF446026 AW118719 AI332765 AW500888 AW576556 AI859571 AW499664 AW614573 AW629495 AW505314 W74704 |
| | | AI356361 AI923640 AW070509 AI521500 AL042095 AA609309 AA761319 AI381489 H45700 AA761333 AW265424 AA909524 AA635311 AA649040 AI392620 Z40708 AI985564 |
| | | AW263513 AA913892 AI693486 AW263502 AI806164 AW291137 BI061872 BI059498 AA134476 AW084888 AA036967 AW370823 T55263 BI002756 AA489664 BF82726 W74741 |
| | | BF963166 |
| 400205 | 2538_1 | NM_006265 D38551 X98294 BM477931 BM461566 AU123557 AU133303 AU134649 AW500421 BM172439 AW500587 AW503665 |
| | | AW504355 AW503640 BM152454 AW505260 AI815984 AW504075 AW500716 AL597310 BC001229 BM474371 AA984202 AU135205 |
| | | BE090841 AW163750 BF747730 BF898637 AI206506 AV660870 AV692110 AW386830 AV656831 N84710 AW993470 BF086802 |
| | | BF758454 BG960772 BF757769 BI870853 BE018627 C75436 AW148744 BF757753 BG622067 BE909924 AA708208 BG530266 BF968015 |
| | | AW992930 BF888862 BG536628 AA143164 AW748953 BG498922 BF885190 BF889005 BF754781 BF800003 BM476529 AI627668 |
| | | AW028126 AL046011 BF590668 AI017447 AA579936 AI367597 AA699622 BE280597 AI124620 AI082548 AW274985 AA677870 AI056767 |
| | | BE551689 AA287642 H94499 AI752427 AI652365 AW002374 AW062651 AA360834 N68822 AU135442 AU125960 Z78334 BE545813 |
| | | AI092115 BF312771 BF242859 BG533616 BG533761 BG164745 BG492433 BM473183 AA172043 AA172069 AU157092 AU151353 |
| | | AU155318 BE302211 AI375022 AA085641 AU157923 H88858 AA132730 AA115113 AA909781 AI475256 AA424206 AW572383 AW084296 |
| | | AI184820 AI469178 AA782432 H92184 AA340562 BF195818 AA852821 AW576342 AA827107 AA173317 AW190014 AI918514 AA729372 |
| | | AA729718 AI055958 AA331424 BE328601 AA515690 BI018896 AW628277 AA748368 AA626222 BG492636 AW380620 BF800058 |
| | | AW370956 AA290909 R25857 BG952995 BF801437 AA172077 AU155890 AU149783 AI720904 AA902936 AA865727 AI470830 AV740677 |
| | | AA142982 AA482485 AU145485 AW576399 AU156042 R63448 BF246427 BE928472 D25910 BF758439 BF968785 BE565238 AA355981 |
| | | AI905607 BG291148 BG533096 BG532888 BF030886 BG613756 BE928471 BG574501 AA187596 AA361196 T95557 BG531446 |
| | | BG527242 BG527513 BG611106 AA085995 BF847252 BG024608 BE540261 BG531236 AL579993 BG108733 BG483503 BG571032 |
| | | BG492505 |
| 400291 | 1314911_1 | AA927862 AA401369 AI873274 |
| 443695 | 20416_12 | BE535598 AW204099 AW301249 AA609749 BF917914 AA775742 AV646137 AV646389 AA314747 |
| 437834 | 294580_1 | BG110129 AW749287 BE535498 AW749299 AW749293 AW749302 AW749298 AW749291 AW749294 AW749289 AW749288 AW749296 |
| | | AA769294 AW749297 AW749295 AW749292 BE002573 |
| 433023 | 3970_8 | BE999967 BF438599 AW864793 AI802899 BE815132 AW468888 AI672189 AI052004 BF112024 AA772335 AW275054 AA573845 |
| | | AI144148 AI968683 AA846676 AA927355 H80424 AW973295 R88209 F29868 BE928871 |
| 427365 | 1314911_1 | AA927862 AA401369 AI873274 |
| 415989 | 10194_1 | BC013389 BC017398 AI023543 AA191424 AI267700 AI469633 AW958465 AW953397 AA172056 BE940298 BF909208 BF909980 |
| | | BF095153 BG285837 AI720344 BF541715 AA355086 AA172236 |
| 458098 | 23945_1 | AI082245 BE467534 AI797130 BE467063 BE467767 BE218421 AI694996 BE327781 BE327407 BE833829 AA989054 AA459718 BE833855 |
| | | BE550224 AA832519 AF086393 AV733386 BE465409 N29245 W07677 AA482971 BE503548 H18151 AA461301 W79223 W74510 |
| | | AI090689 AL600773 AL600781 N46003 R28075 R34182 BE071550 AW885857 AI276145 AI276696 H97808 N20540 AI468553 |
| 412537 | 14066_1 | AK025201 AA425472 AI694282 BG057305 AA907787 AI286170 AI684577 AJ420494 AI809865 BF058095 AI478773 AI160445 AL044114 |
| | | AW665529 AI129239 AW297152 AI268215 AI469807 AI969353 BE552356 N66509 AA736741 AA382555 AW075811 AV759188 BI259364 |
| | | BF445142 BG232065 AI141758 AI631202 AI167566 AI208445 AA889823 BF982682 N90322 BI090882 BF208005 AW953918 AL044113 |
| | | AI016793 AA382556 AW235763 AA927051 AI862075 BE886691 BE619282 |
| 451752 | 10408_5 | AB032997 AI141678 AW978722 BE467119 AI761408 BF727385 AW237035 AI934521 BF436248 AI479668 Z40632 AA832081 AW295901 |
| | | BF057835 BE465977 AI621269 BE465983 BF756369 N74056 AI817896 AA716567 AA934774 H62600 H09497 BF943762 BE395335 |
| | | BE883333 |
| 444172 | 49300_2 | BF526827 AA513594 AL515291 AV648373 AV648176 AA916789 BF002906 AW469960 BE466943 AI367749 AI559715 BF431260 |
| | | AA937968 AI422252 AI288937 BF962778 AA909144 BF960004 BE671534 AI271719 BF925335 BE669504 BF433431 BF924838 AI218062 |
| | | AW960577 AV722716 BI859067 BF944964 BE147740 BF938993 |
| 452279 | 11990_1 | AF392454 AK023074 AI884890 AI814455 AW966220 BF736545 AA026021 AA286843 AA251918 BG197710 AA026294 AA337356 |
| | | BC010422 AK023226 NM_022776 BM459496 AA769310 AI826460 AU153650 AU160375 AW166211 AW292992 BF433538 AI823888 |
| | | AI684798 AI655985 AW770982 AI400454 AI276257 AA639510 AI689818 AW772604 AA807639 AU130298 AU132028 BF900889 BF904822 |
| | | AY007102 AU143256 BG621460 AA829630 AI864665 AI084922 AA025234 AI360060 AA766554 AA026295 AA825817 AA251762 |
| | | BE180751 BM464530 AV715833 AA779447 AI452519 AW418525 AA435643 D25894 AA435651 AA286844 N64369 AI702262 BG288063 |
| | | BE170545 BM466232 BE299160 AA169573 AL567428 BF217285 BE967276 |
| 400277 | 170_1 | Y00281 NM_002950 BC010839 BC007995 BG675232 BM468552 AL555484 BG831516 BF035300 BG677277 BF852972 BE314901 |
| | | BF850656 AI371816 AA292474 AA375747 AA308414 BM454544 BI333370 BM049921 BI461428 BI465007 BI223401 BE856245 AW821 164 |
| | | BF914775 BF914761 AU125835 BI222678 BI091137 BF340536 BM462798 BI224452 BG707915 AL569160 AA443815 AW572867 |
| | | AW363410 BF739268 BG010283 BI013120 BF818845 BF763468 AA305165 AI630370 AA039826 R24906 H02046 T96891 BF981330 |
| | | AW936510 AA478169 H04587 BG166574 BI869342 BE562482 BE539637 AA165089 AL579118 AL553699 BE044054 AW117440 AI520674 |
| | | BF435417 AW245648 AI952404 T29534 AU153459 AU152168 AW591591 AU146918 AI393187 AA478013 AU148143 AI224471 AI640728 |
| | | AI871537 AW264752 N93787 AI189357 AV756134 AI471659 AU147466 AA779206 AU149419 AU149104 AU159135 AA312221 AW445119 |
| | | AW021912 AI799771 F04407 AI285530 AI914643 AW068751 AA513325 AA164627 AA639285 AA569644 T96892 AI923594 BF439180 |
| | | BI770936 BF032438 AU154884 AA682793 AW072992 AU158815 AI884444 AL048031 AU158922 AU152546 AI695187 AL048033 AI245650 |
| | | AU148507 AW467451 BE536868 BF913001 BF062707 AL573082 AW067993 AA523354 BE886727 AI890705 AU159092 AI982693 |
| | | AI817553 AA236729 AI687858 BG163767 AI524675 AI678155 AA127100 AI762661 AU159718 AI469720 AA483627 AW131696 R26868 AI199885 AW875614 AW938694 |
| | | AW578974 BI763988 BG819168 BE874767 BG978292 BE162948 AL555483 AW189719 T56783 AI018819 |
| | | AI476552 BI492837 AI824440 BG996262 AA932887 AI380726 R79530 AA622108 AI262575 T56782 R27437 BE784153 AW129549 |
| | | AI675567 AI866759 BG987935 |
| 446999 | 70312_1 | AA151520 AI749635 AA149436 BE172702 AW317084 AA922501 AI302818 AI147563 AA789216 AI719336 AW612978 F34536 AI971386 |
| | | AI246525 AI183312 R02554 AI360172 AA634282 AI022935 AA639461 AI086411 AI087086 AA633082 AI590029 AA856582 AW369734 |
| | | AA150042 AA877171 AA456459 AI078529 R83333 AI161298 AA056987 AI350120 H88127 AA258759 AI673598 AA454566 AI342790 |
| | | AI492606 AI159945 AI198009 AI198039 AI142751 AI141403 T81478 AW014110 AI197808 AI927796 AA534936 AA649735 AA649697 |
| | | AI349452 AA719660 AW954285 |
| 418304 | 1093209_1 | BE883520 BI057842 AA215702 AA215703 AA368006 BE006876 BE066555 |
| 438869 | 52134_1 | AF075009 R63109 R63068 |
| 406685 | 0_0 | M18728 |
| 443687 | 1347_10 | H72674 BG925294 AA699932 BF061529 AI240821 AA676904 AI083582 AA766036 AA761066 AA761228 AV686983 AV646138 AV646121 |
| | | AW961968 AA004250 AV658573 T08315 AA004669 |
| 453160 | 6028_5 | BC009612 NM_003526 BI597616 AV761592 AV760377 AL601008 BI604131 BE645918 BG187760 BG181525 BG210634 BG192999 |
| | | AI263307 AA344186 AW952966 AA033609 AA037562 AA722183 R79452 H70775 BF674991 BE769437 BG007856 AA037483 AW572535 |
| | | AI143991 AA084581 AA033610 AV742510 AV735788 R08336 |
| 438118 | 1007899_1 | AA778411 AW663081 AW753311 |
| 447349 | 1063443_1 | BE743847 AW809603 BM469626 AI375546 |
| 438714 | 2576235_1 | AA814859 AI582623 AA814857 |
| 454453 | 8582_4 | BF313069 BE879305 AW752781 AW752727 AW752559 AW752578 AW752584 BF846118 AL545903 BF846115 AL525361 |
| 441128 | 20932_1 | BC014072 BE328850 AI356567 AI148171 AI022165 BG149661 BF000671 AA233101 AA573721 AA447991 AW016855 AI005068 AA554071 |
| | | BF478215 AA906902 AW014761 BE905651 BE512923 BM047129 AA243852 AA232991 AA127550 AA127551 AA570256 AI473237 |
| | | BF033706 N90525 AW973623 AI359627 BG674574 BE903322 |
| 411605 | 10026_3 | BG256892 H10532 N46614 R52610 AW977696 BM460488 W56819 BI042183 BG977498 BE767451 BF870009 BG477472 R61137 R14274 |
| | | R20259 R09686 BI838226 BF034269 AA429173 BE741829 AW867495 AI123683 AW006831 BE831162 AW452753 AV742717 W86152 |
| | | BF115102 AI633815 BF921562 AA094230 BE092587 W86151 AA526153 AI672156 BF914496 R12579 BF852352 AA699780 T57386 |
| | | BF903022 R09933 AA678298 |
| 427521 | 513212_1 | AW973352 BF222929 AW016853 BF059130 AI651829 BE551767 AA558414 AI339359 BF059601 AI961162 AI341422 AI206248 AI206165 |
| | | AA548736 AA768578 AI539081 AW025957 AA736837 N79575 AW594357 AA480892 |
| 400247 | 2764_1 | BC022339 BC009610 BC010537 X79805 NM_006713 U12979 BM467814 BM450743 AU132951 AU137129 BG493425 AV758819 |
| | | BG708412 BG705885 BG702217 AV716638 BG777009 BI545689 BI552153 BM476712 BG770858 BG527656 BG528277 BG391388 |
| | | AV716861 BI602926 BG290073 BI667399 BM451469 BI667173 BI602139 BG532171 BI669216 BI544727 BG721852 AV716503 AV701327 |
| | | BM090738 BI492000 AI308856 BI544904 AL599813 AV715829 AV716505 AV714587 AV717902 BF668072 AV716385 BI461927 BM090954 |
| | | AV717826 BG503676 AV647719 BG501392 BG428433 BE895629 BM313117 AW021050 BG435032 BM152910 AA313503 AA872377 |
| | | BG574714 AV712054 AV732696 AA252476 AV712759 AL599643 BE790872 BG654930 W73337 AW675377 AV760376 AV725139 |
| | | AV716379 AA887165 BE830003 AW023796 AL599291 AI902948 BG944042 F00781 AA352483 BG217897 N33888 AW581924 BG654730 |
| | | D31410 AA353088 D31288 AA295029 H95170 BE935104 AU139980 BG772963 BG776470 BG532512 BG105449 BI545421 AV715456 AW386083 BG699714 AL535832 AL514940 |
| | | BG190861 BG210593 AW999254 H95138 AA353863 BE764809 N50375 BE091363 BG701255 |
| | | BI860846 BI832485 BG168150 BG028647 BE546301 BG900321 BI909737 BG702363 BG614141 BG611137 BG700121 BF031492 N85802 |
| | | AV715940 N51590 BG993478 BE172016 AW893622 |
| 413413 | 26025_3 | BG261240 BG231707 AI440201 BF540868 BF240794 BE779394 BG505096 AA129208 D82298 T10758 AU155599 BF857123 BF836348 |
| 429978 | 35194_2 | BE738425 BE738323 BM126944 AW629678 AW265195 AI916735 AI394255 AI573090 AI354442 AW612857 AI339558 AI919424 AI377532 |
| | | AI354441 AI308821 AA772275 AW055215 AI589705 AI336532 AA806547 AV682125 H93575 AW071172 AW769904 AI863985 AW265018 |
| | | AW196655 D79662 BE042393 N75017 AW014741 C75509 BE748621 H92431 AW079261 AW901780 AA329482 AW960115 BI260621 |
| | | AI767525 R31663 BI918664 AW963196 C06195 AI678018 |
| 410507 | 4450_2 | AK027433 AF117064 NM_013319 BC004468 AI149901 AI150093 AI374696 AI566580 AA779898 BG696067 BG828923 BM051241 |
| | | BM050350 AL580560 AL558826 BG182261 BG194259 BG194258 BF036155 AI026803 AI024570 AA702281 AI566953 AW662600 |
| | | AA463546 F33147 AA357796 BE312357 AL516788 AW958856 BE730432 H85868 AA046292 BG478025 BG112231 BG763623 AA098922 |
| | | BI093481 BE746381 AW962126 AI040821 BG026983 AA355288 BE392859 AA085571 BF875433 BF316280 BG740116 BG166624 |
| 441102 | 25369_9 | AK054859 AA973905 AI299888 H63235 AA917019 AV722171 BE514107 BM051585 BE513744 BE295938 W32901 AA649212 |
| 439574 | 689966_1 | BG532820 AW246001 AI469788 AI350090 AI446788 BE549330 W84862 AA837988 |
| 407242 | | |
| 450375 | 16559_3 | BG570706 BG572749 AW606284 H04021 AA151166 AW954405 AA131254 BG056461 W46291 H01532 H04384 H03231 AA852876 |
| | | H04410 H59605 BE157601 AA113758 |
| 432908 | 452541_1 | AF150424 AI861896 AA570057 AV738855 |
| 442643 | 2736_1 | BC001588 BC007424 AF016369 NM_004697 BI756186 BE257019 BG500792 BI862776 AL121371 BG574833 AA703250 AA179511 |
| | | AW052006 AI280150 AI914000 AI358319 AI081204 AI082594 AA992449 AI470821 AI655744 AW237529 AA678858 AI984430 BF433055 |
| | | BE467594 BE467573 AA035630 AI289987 AI184802 AI681391 AW592416 AI138377 AI139266 AA961714 AI800163 AA418751 AW451928 |
| | | AA668676 AI273444 AI494387 BE046912 AI276555 BF196021 AA700055 AA609305 AA772596 AI635758 AI635749 H95459 AW610290 |
| | | BE464994 AA527136 BF374802 AI800175 AW195227 AI189676 BF802049 AL513632 AL554911 AL538845 BE297273 AA315321 |
| | | BM451920 BE269268 BE292835 BE018128 BG755713 BM041095 BG677009 AL039691 BF995709 BE735586 BE296453 BG393609 |
| | | BG824453 AL567522 AI745257 AW388641 AW301265 AI141144 AW029280 AU149362 AU152328 AA418960 AL121009 AI890398 |
| | | AL528748 H13050 T47086 BI000575 BF334914 BF109661 R44450 H13259 T47087 AW388646 BF305834 AL577515 BM041600 BE889299 |
| | | BF239768 |
| 419544 | 236329_1 | BE787623 AI909153 AW362351 AW362269 AW362245 AA526337 AA244193 BF876842 R79395 AI909154 |
| 455286 | 1149378_1 | W27935 AW887403 AW887474 BE144384 BE144386 |
| 400240 | 27222_1 | U04209 NM_005926 AU121948 BI089933 BG425282 BG686931 AW821182 AU100079 BF700972 AL565099 BG116731 BG200436 |
| | | AL555789 AL047536 BF197395 BE504960 AI304957 AA932419 AW575886 AI916292 BI033772 BG995988 AL570315 BE348695 AA331214 |
| | | AL514367 AA436797 AU134683 AL533163 BE818949 BE818946 BE818920 AU136916 AL047535 BG424079 BE294696 AU135607 |
| | | BE207326 AL514368 AA318042 BF831066 BG106341 AW865170 BE937045 AI828674 BM083453 BE385535 AW865372 BF350485 AW382531 R01323 AA092464 |
| 455290 | 1151240_1 | U75810 AW890252 |
| 449570 | 79253_1 | AA001793 AA001871 |
| 448663 | 16112_4 | BE966763 AA659765 AI961656 AI520918 AA761743 AA281477 N66431 BE463652 AA281329 AW272944 AA058687 BM145087 AA045516 |
| | | AW341820 AA112515 AA258766 AI886639 AA714133 AA768245 AA035533 AI630459 T20165 AW971268 BE966269 AA522722 |
| 433009 | 2142268_1 | AA761668 AA573621 R09670 R92814 |
| 447197 | 2176805_1 | R36075 R36167 AI366546 |
| 432586 | 6633_1 | BC022881 AU150944 BG750783 AW754175 AW857737 AI911659 AI050036 AA554053 AI826259 AA568548 |
| 449444 | 2735_1 | U59185 NM_004696 AV734324 AI245349 AA369517 H88760 D79128 AA970406 H01059 H88761 H03446 BG620383 AU135008 AU136895 |
| | | AU158158 AU155762 R73608 R65751 R23756 N74630 AW078687 BE439761 BE786351 R68994 BE785867 AW297502 AW297553 |
| | | BG431545 AW814843 BF382644 BG429539 BE929862 BF811258 |
| 438578 | 2573130_1 | AA811244 AI373188 AI240509 |
| 441318 | 1989216_1 | AA928233 AI078234 AI702558 H81727 |
| 442961 | 60316_2 | BE966247 BE220885 BE467384 BE350135 BE672094 AI811582 AW665254 AA772731 AI283601 AA417067 AW197746 AI868357 |
| | | AI792143 AA931120 AI758506 AA843761 BE737582 AW379586 N38812 BG567321 H13257 |
| 450251 | 40382_4 | BE080483 AI689298 BE080416 |
| 422150 | 782_1 | BI086421 BF059136 AW003898 BF446659 AI632891 AI628067 AI703179 AI961149 BF111022 AW614154 BE674215 AA687350 AA779426 |
| | | BF591963 AW243344 AI356530 AI492508 AI694049 AI090422 AA465307 AI273387 BE674625 AW271971 AA969153 AW468593 AA984014 |
| | | AI817491 AA970258 AI914450 AI018697 AW577591 AW577616 AA382101 AW954455 AI867266 AI707995 AI337384 BF208406 BF037100 |
| | | BF223433 BF195517 BE673984 BF224124 BE813387 BG036579 BI553906 AA304995 AW361269 AW754160 AW361276 AW361271 |
| | | AI867118 AW805555 AW361284 AW954458 AW958551 BG681507 H79011 AW205696 AW134957 AA747667 AW753296 BF939060 |
| | | AW958549 BF910827 BG573750 BG168639 |
| 418330 | 36528_1 | AK056357 BG704214 BG714527 BE409405 BE514455 BE408705 BM351826 BM049253 BM352137 AI659121 AW074357 AI674368 |
| | | AW207841 BE646201 BM453984 BE674342 H19667 AI458175 AA279585 AW438632 AI687141 AA433999 AI245284 AA366782 AI948491 |
| | | AW450917 AW450396 AI949813 BE217772 BE504863 AI628938 BF059575 AI564121 AI073854 AW510570 AI769861 AW235541 |
| | | AW771224 AW236106 AW074675 AI919535 AA977064 AI364994 AI631614 W58249 AI034276 BE169956 AA743584 H19668 AI222131 |
| | | AA883261 AI351534 F10472 AW466882 AA814964 W58250 BF036304 BF932025 BM474362 BG110744 AW964083 AW369992 F12868 BF842785 AA356665 BI091945 BG059147 |
| | | W04844 R18398 BE925184 BI050068 BI825078 BE784883 AW175845 AA216130 BI257636 |
| | | AA504328 AA713664 BF754840 AW235144 R38555 R42461 BG676504 T75135 R15365 BM012684 |
| 454645 | 1065291_1 | AW811928 AW811887 AW811948 AW811847 |
| 455540 | 430605_1 | AW993000 AV720038 AV720142 AW993293 BE080231 AW993284 |
| 424339 | 50559_1 | NM_015720 AF219137 AL534420 AL524055 AL537346 AL538442 BG765888 AL530054 AL525377 BG474596 BG473144 BE251553 |
| | | BG706099 AL538039 BG703131 BE255806 BF805256 F12128 AL566773 BI828686 BF761480 AI204971 BG818818 BI199246 AL534816 |
| | | BF529941 AA324163 AL523285 BG914330 H07952 AL534815 BE769903 AI867802 BM310135 AL533702 BE254484 BF528852 BE867462 |
| | | BE740130 AL134164 AL567115 AL533701 AL524054 AL515904 AL523284 AL568203 AL534419 BF981162 BE257148 AL561833 |
| 408839 | 234669_1 | AI355222 BF378422 AA366587 BF874552 AW277084 R26970 D79194 R27662 |
| 414883 | 8371_2 | AF274943 BG494894 AI719075 AA908783 AI935150 AI422691 AA910644 AA583187 BM272167 AI828996 AA527373 AW972459 AI831360 |
| | | AA772418 AI033892 AA100926 AU154749 AI459432 AI423513 AI094597 AA740817 AI991988 AI090262 AI312104 BI256707 AA459522 |
| | | AA416871 AI075239 AI339996 AA701623 AI139549 AI336880 AA633648 AI989380 AI362835 AA399239 AI146955 BF514270 N92892 |
| | | AI348243 AI278887 AA459292 AI494230 BF507531 AI492600 AA962596 AW613002 AA293140 AA235549 BF108854 AA954344 N49682 |
| | | AI457100 AW589407 AW300758 BE220715 BE220698 BE569091 BM009647 BF900351 AI537692 AI203723 AI857576 AA584410 |
| | | AW371667 BM172363 |
| 453454 | 2736_1 | BC001588 BC007424 AF016369 NM_004697 BI756186 BE257019 BG500792 BI862776 AL121371 BG574833 AA703250 AA179511 |
| | | AW052006 AI280150 AI914000 AI358319 AI081204 AI082594 AA992449 AI470821 AI655744 AW237529 AA678858 AI984430 BF433055 |
| | | BE467594 BE467573 AA035630 AI289987 AI184802 AI681391 AW592416 AI138377 AI139266 AA961714 AI800163 AA418751 AW451928 |
| | | AA668676 AI273444 AI494387 BE046912 AI276555 BF196021 AA700055 AA609305 AA772596 AI635758 AI635749 H95459 AW610290 |
| | | BE464994 AA527136 BF374802 AI800175 AW195227 AI189676 BF802049 AL513632 AL554911 AL538845 BE297273 AA315321 |
| | | BM451920 BE269268 BE292835 BE018128 BG755713 BM041095 BG677009 AL039691 BF995709 BE735586 BE296453 BG393609 |
| | | BG824453 AL567522 AI745257 AW388641 AW301265 AI141144 AW029280 AU149362 AU152328 AA418960 AL121009 AI890398 AL528748 H13050 T47086 BI000575 BF334914 |
| | | BF109661 R44450 H13259 T47087 AW388646 BF305834 AL577515 BM041600 BE889299 BF239768 |
| 429588 | 1396089_1 | AI092511 AI080271 AA455177 AI381859 AI381721 |
| 412452 | 71091_1 | BE796667 BF330981 BE394193 Z45547 BG490525 F35734 AA130708 AA577072 AA446587 AA215665 AA978209 BG740729 BG746810 |
| | | BE298184 AI356291 AI671975 AI818924 AV715722 AI078381 BI142391 AI201085 AI198283 AI077572 AI694848 AW016425 BM456416 |
| | | AI277223 AW771476 F26140 AA102778 AW025780 R44726 AA761079 AI581346 AI991909 BM005939 BE537999 BG469717 AA114156 |
| | | BF437200 BE774942 |
| 415889 | 12922_1 | NM_052918 AF284756 BE019093 Z42986 BE328250 BE207835 R54516 R24563 H08127 BI522616 AA551620 H07879 AI174481 BF941262 |
| | | BF222810 R54417 AW137766 AI638502 N22373 H08128 R44366 AW272405 AI675836 Z38786 N75618 F02463 AI654047 BI492031 |
| | | AW021081 |
| 442092 | 22756_2 | BM449821 BM069895 BM023170 AW025563 BG152606 BM023452 AI862106 BG959957 |
| 459376 | 31010_1 | BC002465 BE254864 BG472164 BE258770 |
| 410723 | 6090_25 | AW070415 AA628250 AW204027 AI936167 AW054868 BF805308 AA100683 AA088786 |
| 429462 | 5761_2 | BC008352 BM006782 AW245715 AI890356 BF445407 BE222242 AW139455 BE549685 AA987521 AA960934 AW245359 AA453285 |
| | | AA976507 BG055359 BM007094 |
| 424399 | 2196_1 | NM_058173 AF414087 W72837 BF742809 AW070916 BE092421 AI905687 AA340069 BE074512 AI905623 AI905633 BG202312 W72838 |
| | | AI139456 BG218084 BE926938 BE186013 AW176044 AW291950 BG185269 BG197186 BG192597 BG183176 BG207535 AI127172 |
| | | BE815819 AI905624 R75793 BG202313 AI905837 BE815853 |
| 440282 | 21425_1 | NM_021188 U90919 BM458077 BG214941 AL562127 BG197712 AW994207 AA043114 AW993474 AA631832 AI911529 BI001014 |
| | | BE156322 BG319454 H27376 BG220618 BG189474 BG325006 AL519085 BM472894 AL520560 BG575806 AA319317 BG621802 |
| | | BG912722 AV760869 AV760871 R13243 BE274097 BG327062 BI765172 BE614338 BG214923 BM153391 AU147675 N58119 AL043557 |
| | | AL043558 AL043510 AA773474 AU148079 BF062869 AW189972 AW250398 AI028605 AI434688 AW149344 AI246783 AI961376 AI698209 |
| | | AA251535 AU160747 AA579811 AI572570 AI656228 AA426093 AA127171 BF222487 AA725576 F21161 AI932217 BF222251 AI024660 |
| | | AA780998 AI538862 AW140038 AI122909 AA493515 AA401920 Z39789 F10942 F05081 AA814868 F10376 AA776314 AA993104 T94991 |
| | | AW583990 AA128211 F03074 AL579261 AL519084 AA384895 BG196670 AW083526 BF220181 BE669988 BF907279 T23499 AL520559 |
| | | AU149005 N38766 H96506 AA040865 AI263737 AA723350 AI168145 AA652386 AI374699 AA487377 AI927374 AI985531 AI352105 |
| | | AI040092 AA486562 BM053020 Z41738 BE243710 AA601558 BM052743 AI445827 AA487260 AI982679 AW130546 AI743384 AI335647 AW978172 R40324 AV736436 |
| | | BE966360 AI185467 AI367193 N75025 AW131068 BE940519 H43910 AA486659 BE614365 BF220261 |
| | | BE181357 BG189473 BG210176 H43958 |
| 412988 | 1342150_1 | BE046680 BE046738 BE044958 |
| 435542 | 132718_1 | AW975503 BE763276 H74234 AA687376 |
| 418647 | 243680_1 | AA226513 AA383773 AA226198 |
| 424765 | 6857_1 | AK021881 AU145974 AU145787 C16964 AA428211 AU119698 AA993264 BF999192 AW903017 AA346559 AU119446 AW581679 |
| | | AA991677 AW898165 AW386878 AW890957 Z18340 |
| 439306 | 3941_3 | BM013864 AI905414 AF086118 BE178507 AW161170 BE220199 BE221405 W01813 BG150053 N70760 N26062 H98499 AI290198 |
| | | T80987 BE549149 BG106008 AW662681 AI590833 T71977 |
| 422405 | 17366_3 | AW006867 AI373549 AA310278 |
| 400203 | 11774_1 | NM_002794 D26599 BM469989 BF305151 BG821966 BI089030 AW007738 BI222910 BM049422 BG028749 AI189162 AI831230 |
| | | AW131497 BM272215 BE791105 AW778828 AA479594 AA480133 AA131997 AA284572 AA453009 BF928258 AA152127 AA393918 |
| | | BF765307 BE247542 BF934697 BF341798 BE253409 AA470620 AI828932 AW379902 AV762678 AV741784 AV760892 AI025755 |
| | | AA878562 AA630630 AA761708 AA862518 AA865831 AA862947 N53065 AA131821 AA293499 N23342 N26856 AI147346 AW951549 |
| | | AA772963 BE245986 BG208493 AI831666 BG474873 BI023168 AU149647 BG197069 BG191102 BF304178 BE536135 AA706900 |
| | | AA443583 AI002710 AW276192 AU149842 BG214797 BG198193 AW197923 AW627799 T98663 BG194788 BG214656 BF345258 |
| | | BG716363 AI066528 BI546220 AA393815 AA132004 AA353826 H97858 BG187823 BF841463 AI351714 AV735966 BG196439 BG216840 |
| | | BG198438 BG400762 |
| 412700 | 18692_7 | BG164322 BF510268 AI681542 BE222433 AI299965 BE220552 AA115728 BE896438 |
| 406740 | 0_0 | AA577274 AA908437 |
| 442481 | 678752_1 | BG398943 N99828 AI110738 AF074645 BE969969 BE079873 BF185244 |
| 430677 | 11749_1 | NM_001943 Z26317 BG750290 BM043721 AW361908 BG494570 AW996792 BF915903 AW753487 BI335109 BE018413 BE874074 |
| | | BF333555 BI016581 BI014002 BI015917 BE731644 BE927177 BE697899 BF999996 BF929423 BG951608 AW858747 AW858755 |
| | | AW858750 AW858749 AW858751 AW363742 D58979 BE003946 AW858999 BG951830 AW859003 BF738953 AW369461 AW363740 |
| | | AW998641 BG978975 BG015802 AW991316 |
| 453331 | 16559_1 | BG571303 AA410586 AA035018 BG572117 BG620022 AA147247 BG005785 BG014448 R31981 H02668 H12498 R36203 BF992089 |
| | | R73999 T49904 R75732 BI057974 T53681 AA147933 N50695 R68588 R25671 R31935 R25110 R36105 AK055628 BE157467 AW663674 |
| | | AA190993 H01642 BF510304 AA626915 AA746952 AI161014 AA099554 BG572534 AI803329 AI809932 AI808765 AA411449 AI378760 |
| | | AA976929 AI378620 AA909684 R75632 AI360919 AI350463 AW069127 AA411621 AA742532 H12451 BE208298 H03612 H12839 N58781 |
| | | R75957 BF996484 AI240665 BF989591 BI056086 BG001590 BF107035 |
| 409960 | 39576_1 | BE644758 AI082238 BF940027 AI201079 AI436035 AW275966 AI085394 AI291655 AW070441 AI474134 AI268978 AI769279 AI567682 |
| | | AA693941 BF477668 AW664149 AA283782 BF509538 AW296868 AI268977 AI168133 BM352065 AI262769 BF941976 AI056920 |
| | | AA481861 BF763697 AL565888 BM352383 AA427768 AA385346 AI186988 AA931831 AA134972 BF217480 BF111012 AA908246 |
| | | AA319849 AA318136 AL514271 BF364291 AL515057 AV702312 AA377395 AL544217 AI341000 AW193583 AI350789 AA888338 |
| | | BF945380 AW879092 AA130839 T91066 N92326 AI004389 AA078832 AL572370 W04622 BE314003 AW960808 BM360872 AA319160 |
| | | AA130778 AL514257 |
| 408673 | 13031_1 | BG027435 BF746745 AF349017 NM_052963 AL553524 AL519055 AL525199 AL529016 BE208517 BI856042 BG480978 BE267120 |
| | | AW815679 AL525198 AL561760 AA933030 AA634332 AA568280 AA927487 AW182340 AW592604 AW732157 AI680072 AI431328 |
| | | AI357601 AI872335 AI032112 D25780 AL529015 AL576497 AA575842 BG223081 AA618007 BF310845 BF757605 BM012536 BI911537 |
| | | BE964237 |
| 447128 | 11028_3 | BG292064 AI452509 AI271898 BE048502 BI966153 X84721 AI858001 BM021943 AI553937 AI765259 F25787 AW015380 AA554539 |
| | | AW059537 F35749 AA149853 AA961610 AI568815 AW973696 C00201 R77127 BG438065 AI244810 H00719 |
| 400249 | 993_1 | BG260581 AI743827 AA182444 AA927609 NM_001326 U15782 BC010533 AA779834 AI086366 AI452475 AW274511 BG056719 |
| | | AW026350 AA808891 AW080007 AI763436 AU154714 AU155464 BF196839 AI934353 AI376072 BG232033 AI040445 AA700556 |
| | | AW004704 BE047781 AA470756 AI091381 AI302228 AI400050 AI142702 AA614554 AI467907 AA282801 AI434140 AI357496 AA748501 |
| | | AA430113 BF060907 AW207004 AI367341 AA873520 AA764823 AI077410 AA253061 AI052369 F08358 BM456285 BI518533 BI836074 AU133365 AU131081 AU127466 |
| | | AA173834 AW999116 BG571523 AU100072 BG290403 AI743461 W90141 D58551 AA181551 AA094014 BG724183 |
| 439092 | 919640_1 | AW978407 AA830149 M85983 AW503637 BF352096 |
| 407590 | 78212_1 | BE293321 AA631602 AA281584 AA927393 AI831258 AA340072 BG009209 AW956385 AW838390 N94346 BF724763 AW082728 |
| | | BE044419 AI417373 W69444 AI147614 AA632403 AI937606 AI864878 AI969211 AI081135 AA885000 AA926984 AA642586 AW079405 |
| | | AA973001 AW628858 AI521070 BG912959 BI052498 |
| 424036 | 6226_1 | NM_033445 BC001193 AI885781 BF794032 AA476620 AA810906 AA810905 AI291244 AI885097 AI359708 AI335629 H97396 AI344589 |
| | | AA300377 AA457566 AW771833 BE465621 AI364068 AI364452 AI648505 AI918342 AI928670 AA886580 AI531029 AA886344 AI186419 |
| | | BG329096 BM045465 AL531028 BG437151 BE868021 AA179427 |
| 408756 | MH1740_17 | AA524743 BF515403 BM353526 AI803357 BF000688 AW665479 AI672715 AW150325 AW589318 AI693038 AI370572 AI167636 |
| | | AA057236 AW439079 AI097376 AI382060 AI684871 AI684860 AA913419 AA806162 AI673426 BI324967 BM149631 BM145789 BM353796 |
| | | AA913878 BM148707 N38884 BI761934 BM145978 BE243268 BE246569 N38883 |
| 449230 | 6182_6 | BC006329 AK026224 BG105365 AI472084 AW074277 AI743908 BM309990 BI850432 AI094365 AI539568 AW779999 AI261365 AA988975 |
| | | AI436272 AI559886 BI710742 AA988508 AA036678 AI867147 AW518513 AI620019 BE645777 AI801919 AW205320 AI457722 BF061996 |
| | | BF061798 AA233854 AA232795 AA516294 AA830561 R74220 BG768337 AW392972 BE764979 BE257665 |
| 414390 | 2128_2 | BC022204 AK027690 NM_032839 BI765386 BE903404 BG700172 BF701671 BG252600 W60255 AW301576 W01296 AV724003 |
| | | BE999965 AI949788 BM040799 Z43693 BF082768 BF328302 H09192 BF332781 R34999 BG573394 N57281 BE009522 BE281040 |
| 458422 | 4340_3 | NM_006260 BE048475 AW080036 AA287317 AA400028 AI204437 AI830642 AA644420 AW614662 AW261942 U28424 BG335330 |
| | | AU148480 AI095508 BF727387 BG256497 AI521859 AW291686 AW007816 BG002833 AA853075 AA779079 BF082050 AI640393 |
| | | AA522954 T55310 AW517649 AA127463 AA887984 AW206341 BE858004 AW772531 AA604169 H83777 BG290990 AI692188 AI223311 |
| | | AI708839 D82262 AA600260 AI364786 AA471007 |
| 434636 | 15423_1 | AJ420454 AF147430 AA910497 BF432963 AI701451 AI743089 AA429326 AI887812 AA315932 AI005464 AL043321 AI300993 AA425105 |
| | | BE467230 BE669770 AA885637 BE503044 AW014324 AI809584 AW167510 AA921331 AA903224 W01644 AI762128 AA031404 BE550653 |
| | | AI694045 BE043088 BE670430 AI630969 AI457315 BE644737 BE327316 AW295247 N92784 AI630807 BE328180 AI269949 AW245292 |
| | | AA083765 AA256898 AI375535 AA430673 AI168735 AI589717 AA015942 AI693885 AW341205 AA931651 BF856764 BE468094 BF433393 |
| | | BF445511 AA928976 AI817684 BF111008 AA428316 AA455858 N25716 AA568727 AI581817 AA427482 H40678 AA041483 N71630 |
| | | H51826 BF969052 AA094470 AI560352 T98937 W52816 AA083764 |

Pkey: Unique Eos probeset identifier number
CAT number: Gene cluster number
Accession: Genbank accession numbers

TABLE 11C

| Pkey | Ref | Strand | Nt_position |
|---|---|---|---|
| 405770 | 2735037 | Plus | 61057-62075 |
| 404561 | 9795980 | Minus | 69039-70100 |
| 401451 | 6634068 | Minus | 119926-121272 |
| 401519 | 6649315 | Plus | 157315-157950 |
| 401866 | 8018106 | Plus | 73126-73623 |
| 403485 | 9966528 | Plus | 2889-3001, 3198-3532, |
| | | | 3655-4117 |
| 401464 | 6682291 | Minus | 170688-170834 |
| 404996 | 6007890 | Plus | 37999-38145, 38652-38998, |
| | | | 39727-39872, 4055 |
| 402802 | 3287156 | Minus | 53242-53432 |
| 402408 | 9796239 | Minus | 110326-110491 |
| 401558 | 7139678 | Plus | 103510-104090 |
| 404632 | 9796668 | Plus | 45096-45229 |
| 404571 | 7249169 | Minus | 112450-112648 |
| 405558 | 1621110 | Plus | 4502-4644, 5983-6083 |
| 406081 | 9123861 | Minus | 38115-38691 |
| 402496 | 9797769 | Minus | 8615-9103 |
| 403011 | 6693597 | Minus | 3468-3623 |
| 401203 | 9743387 | Minus | 172961-173056, 173868-173928 |
| 404440 | 7528051 | Plus | 80430-81581 |
| 401435 | 8217934 | Minus | 54508-55233 |
| 406581 | 7711879 | Minus | 12694-12852 |
| 404607 | 7328770 | Plus | 28477-28591 |
| 401093 | 8516137 | Minus | 22335-23166 |
| 405366 | 2182280 | Plus | 22478-22632 |
| 404819 | 4678240 | Plus | 16223-16319, 16427-16513, |
| | | | 16736-16859, 1694 |
| 400880 | 9931121 | Plus | 29235-29336, 36363-36580 |
| 401197 | 9719705 | Plus | 176341-176452 |
| 404580 | 6539738 | Minus | 240588-241589 |
| 402102 | 8117771 | Minus | 174566-174740 |
| 402812 | 6010110 | Plus | 25026-25091, 25844-25920 |
| 402855 | 9662953 | Minus | 59763-59909 |
| 401405 | 7768126 | Minus | 69276-69452, 69548-69958 |
| 404592 | 9943965 | Minus | 39067-39225 |
| 406203 | 7289992 | Minus | 82220-82639 |
| 404661 | 9797073 | Plus | 33374-33675, 33769-34008 |
| 405411 | 3451356 | Minus | 17503-17778, 18021-18290 |
| 400639 | 9887597 | Plus | 23150-23580 |
| 405705 | 4165009 | Plus | 120228-120800 |
| 403055 | 8748904 | Minus | 109532-110225 |
| 400884 | 9958187 | Minus | 57979-58189 |
| 404877 | 1519284 | Plus | 1095-2107 |
| 401160 | 6067118 | Minus | 8280-8945 |
| 403429 | 9719566 | Minus | 52789-52917 |
| 403154 | 7407986 | Minus | 14228-14736 |
| 401833 | 3063511 | Minus | 77974-78102, 80090-80187, |
| | | | 81695-81773, 8717 |
| 402322 | 7630359 | Minus | 75078-75203 |
| 405204 | 7230116 | Plus | 126569-126754 |
| 403961 | 7596976 | Minus | 110393-110603 |
| 403969 | 8569909 | Plus | 31237-31375, 32405-32506 |
| 401192 | 9719502 | Minus | 69559-70101 |
| 401832 | 3063511 | Minus | 53654-53793, 57497-57647, |
| | | | 60769-60981, 6501 |
| 401797 | 6730720 | Plus | 6973-7118 |
| 404029 | 7671252 | Plus | 108716-111112 |
| 404831 | 6624702 | Minus | 16833-17020, 20007-20120, |
| | | | 21605-21799, 2333 |
| 401463 | 6682291 | Minus | 163135-163262 |
| 403532 | 8076842 | Minus | 81750-81901 |
| 405264 | 7329374 | Plus | 28556-28684 |
| 400810 | 8567959 | Minus | 174204-174331, 175062-175205 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 12A
About 2867 genes upregulated in lung and breast metastases to the brain relative to normal breast and lung tissues

| | |
|---|---|
| Pkey: | Unique Eos probeset identifier number |
| ExAccn: | Exemplar Accession number, Genbank accession number |
| UniGeneID: | UniGene number |
| UniGene Title: | UniGene gene title |
| R1: | 90th percentile of breast and lung metastases to the brain Als divided by the 90th percentile |
| | of normal breast and lung Als, where the 15th percentile of all normal body tissue |
| | Als was subtracted from the numerator and denominator. |
| SEQ ID NO(s): | SEQ ID number(s) for nucleic acid and protein sequences associated with table entry. |

| Pkey | ExAccn | UniGeneID | UniGene Title | R1 | SEQ ID NO(s): |
|---|---|---|---|---|---|
| 406690 | M29540 | Hs.220529 | carcinoembryonic antigen-related cell ad | 45.45 | 4748 86 |
| 409103 | AF251237 | Hs.112208 | XAGE-1 protein | 27.75 | 333 4812 |
| 412719 | AW016610 | Hs.816 | ESTs | 27.70 | 663 |
| 417308 | H60720 | Hs.81892 | KIAA0101 gene product | 21.61 | 1079 |
| 419078 | M93119 | Hs.89584 | insulinoma-associated 1 | 19.40 | 1272 5036 |
| 422963 | M79141 | Hs.13234 | sphingosine 1-phosphate phosphohydrolase | 19.38 | 1701 |
| 452838 | U65011 | Hs.30743 | preferentially expressed antigen in mela | 15.85 | 4353 5765 |
| 428839 | AI767756 | Hs.82302 | Homo sapiens cDNA FLJ14814 fis, clone NT | 15.75 | 2310 |
| 433447 | U29195 | Hs.3281 | neuronal pentraxin II | 15.70 | 2764 5536 |
| 453857 | AL080235 | Hs.35861 | Ras-induced senescence 1 (RiS1) | 15.19 | 4437 5785 |
| 425057 | AA826434 | Hs.1619 | achaete-scute complex (Drosophila) homol | 14.85 | 1915 |
| 449722 | BE280074 | Hs.23960 | cyclin B1 | 14.55 | 4079 |
| 426682 | AV660038 | Hs.2056 | UDP glycosyltransferase 1 family, polype | 14.40 | 2086 |
| 419875 | AA853410 | Hs.93557 | proenkephalin | 14.02 | 1365 |
| 455601 | AI368680 | Hs.816 | SRY (sex determining region Y)-box 2 | 13.85 | 4515 |
| 418738 | AW388633 | Hs.6682 | solute carrier family 7, (cationic amino | 13.85 | 1234 |
| 430130 | AL137311 | Hs.234074 | Homo sapiens mRNA; cDNA DKFZp761G02121 ( | 13.70 | 2445 5436 |
| 428342 | AI739168 | | Homo sapiens cDNA FLJ13458 fis, clone PL | 13.70 | 2244 |
| 422487 | AJ010901 | Hs.198267 | mucin 4, tracheobronchial | 13.54 | 1649 5152 |
| 424001 | W67883 | Hs.137476 | paternally expressed 10 | 13.45 | 1788 |
| 424922 | BE386547 | Hs.217112 | hypothetical protein MGC10825 | 13.25 | 1900 |
| 412446 | AI768015 | | ESTs | 13.10 | 633 |
| 436217 | T53925 | Hs.107 | fibrinogen-like 1 | 12.63 | 2968 |
| 422440 | NM_004812 | Hs.116724 | aldo-keto reductase family 1, member B10 | 12.60 | 1647 5151 |
| 412140 | AA219691 | Hs.73625 | RAB6 interacting, kinesin-like(rabkines | 12.45 | 613 |
| 439897 | NM_015310 | Hs.6763 | KIAA0942 protein | 12.36 | 3241 5600 |
| 427344 | NM_000869 | Hs.2142 | 5-hydroxytryptamine (serotonin) receptor | 12.25 | 2145 5336 |
| 435538 | AB011540 | Hs.4930 | low density lipoprotein receptor-related | 12.00 | 2924 5565 |
| 425234 | AW152225 | Hs.165909 | ESTs, Weakly similar to I38022 hypotheti | 12.00 | 1940 |
| 418329 | AW247430 | Hs.84152 | cystathionine-beta-synthase | 11.75 | 1186 |
| 414219 | W20010 | Hs.75823 | ALL1-fused gene from chromosome 1q | 11.65 | 804 |
| 447329 | BE090517 | | ESTs, Moderately similar to ALU8_HUMAN A | 11.60 | 3842 |
| 426514 | BE616633 | Hs.170195 | bone morphogenetic protein 7 (osteogenic | 11.55 | 2073 |
| 413554 | AA319146 | Hs.75426 | secretogranin II (chromogranin C) | 11.40 | 734 |
| 414683 | S78296 | Hs.76888 | hypothetical protein MGC12702 | 11.40 | 4923 862 |
| 429922 | Z97630 | Hs.226117 | H1 histone family, member 0 | 11.40 | 2427 5430 |
| 439926 | AW014875 | Hs.137007 | ESTs | 11.30 | 3243 |
| 401451 | | | NM_004496*: Homo sapiens hepatocyte nucle | 11.30 | 27 4697 |
| 436291 | BE568452 | Hs.344037 | protein regulator of cytokinesis 1 | 11.30 | 2975 |
| 442432 | BE093589 | Hs.38178 | hypothetical protein FLJ23468 | 11.30 | 3429 |
| 434001 | AW950905 | Hs.3697 | angiotensinogen | 11.21 | 2804 |
| 432415 | T16971 | Hs.289014 | ESTs, Weakly similar to A43932 mucin 2 p | 10.85 | 2665 |
| 453439 | AI572438 | Hs.32976 | guanine nucleotide binding protein 4 | 10.73 | 4406 |
| 410082 | AA081594 | Hs.158311 | Musashi (Drosophila) homolog 1 | 10.67 | 442 |
| 428450 | NM_014791 | Hs.184339 | KIAA0175 gene product | 10.59 | 2259 5359 |
| 407137 | T97307 | | gb: ye53h05.s1 Soares fetal liver spleen | 10.55 | 128 |
| 457465 | AW301344 | Hs.122908 | DNA replication factor | 10.25 | 4592 |
| 457211 | AW972565 | Hs.32399 | ESTs, Weakly similar to S51797 vasodilat | 10.24 | 4583 |
| 439963 | AW247529 | Hs.6793 | platelet-activating factor acetylhydrola | 10.22 | 3250 |
| 422880 | AF228704 | Hs.193974 | glutathione reductase | 10.15 | 1689 5161 |
| 422656 | AI870435 | Hs.1569 | LIM homeobox protein 2 | 10.15 | 1668 |
| 409269 | AA576953 | Hs.22972 | steroid 5 alpha-reductase 2-like; H5AR g | 10.00 | 358 |
| 428931 | AA994979 | Hs.98967 | ATPase, H()-transporting, lysosomal, non | 9.85 | 2317 |
| 446469 | BE094848 | Hs.15113 | homogentisate 1,2-dioxygenase (homogenti | 9.85 | 3753 |
| 410442 | X73424 | Hs.63788 | propionyl Coenzyme A carboxylase, beta p | 9.85 | 479 4848 |
| 411305 | BE241596 | Hs.69547 | myelin basic protein | 9.84 | 546 |
| 407896 | D76435 | Hs.41154 | Zic family member 1 (odd-paired Drosophi | 9.75 | 213 4785 |
| 446839 | BE091926 | Hs.16244 | mitotic spindle coiled-coil related prot | 9.65 | 3794 |
| 416602 | NM_006159 | Hs.79389 | Protein kinase C-binding protein NELL2 | 9.45 | 1006 4965 |
| 432886 | BE159028 | Hs.279704 | chromatin accessibility complex 1 | 9.30 | 2708 |
| 453884 | AA355925 | Hs.36232 | KIAA0186 gene product | 9.30 | 4444 |
| 407355 | AA846203 | Hs.193974 | ESTs, Weakly similar to ALU1_HUMAN ALU S | 9.30 | 155 |
| 442547 | AA306997 | Hs.217484 | ESTs, Weakly similar to ALU1_HUMAN ALU S | 9.25 | 3439 |
| 443247 | BE614387 | Hs.333893 | c-Myc target JPO1 | 9.25 | 3513 |
| 440274 | R24595 | Hs.7122 | scrapie responsive protein 1 | 9.23 | 3275 |
| 409974 | BE174106 | Hs.225641 | hypothetical protein FLJ13171 | 9.20 | 423 |
| 416655 | AW968613 | Hs.79428 | BCL2/adenovirus E1B 19 kD-interacting pro | 9.19 | 1010 |
| 409041 | AB033025 | Hs.50081 | Hypothetical protein, XP_051860 (KIAA119 | 9.15 | 327 4811 |
| 456977 | AK000252 | Hs.169758 | hypothetical protein FLJ20245 | 9.13 | 4572 5807 |
| 410102 | AW248508 | Hs.279727 | ESTs; homologue of PEM-3 [Ciona savignyi | 9.09 | 446 |
| 437387 | AI198874 | Hs.28847 | AD026 protein | 9.08 | 3062 |
| 411908 | L27943 | Hs.72924 | cytidine deaminase | 9.00 | 4869 585 |
| 407168 | R45175 | Hs.117183 | ESTs | 8.90 | 131 |
| 433160 | AW207002 | Hs.134342 | TASP for testis-specific adriamycin sens | 8.88 | 2742 |
| 446619 | AU076643 | Hs.313 | secreted phosphoprotein 1 (osteopontin, | 8.85 | 3770 |
| 451807 | W52854 | | hypothetical protein FLJ23293 similar to | 8.75 | 4249 |
| 452046 | AB018345 | Hs.27657 | KIAA0802 protein | 8.75 | 4266 5754 |
| 418836 | AI655499 | Hs.161712 | BMP-R1B | 8.71 | 1247 |
| 416854 | H40164 | Hs.80296 | Purkinje cell protein 4 | 8.65 | 1031 |
| 454293 | H49739 | Hs.134013 | ESTs, Moderately similar to HK61_HUMAN H | 8.64 | 4475 |
| 427528 | AU077143 | Hs.179565 | minichromosome maintenance deficient (S. | 8.63 | 2161 |
| 431211 | M86849 | Hs.323733 | gap junction protein, beta 2, 26 kD (conn | 8.60 | 2540 5473 |
| 417576 | AA339449 | Hs.82285 | phosphoribosylglycinamide formyltransfer | 8.55 | 1102 |
| 412519 | AA196241 | Hs.73980 | troponin T1, skeletal, slow | 8.51 | 641 |
| 417333 | AL157545 | Hs.173179 | bromodomain and PHD finger containing, 3 | 8.45 | 1081 |
| 431726 | NM_015361 | Hs.268053 | KIAA0029 protein | 8.45 | 2592 5489 |
| 442355 | AA456539 | Hs.8262 | lysosomal-associated membrane protein 2 | 8.44 | 3424 |
| 412482 | AI499930 | Hs.334885 | mitochondrial GTP binding protein | 8.40 | 636 |
| 429503 | AA394183 | Hs.204166 | ESTs | 8.40 | 2381 |
| 429343 | AK000785 | Hs.199480 | Homo sapiens, Similar to epsin 3, clone | 8.39 | 2356 5402 |
| 427871 | AW992405 | Hs.59622 | Homo sapiens, clone IMAGE: 3507281, mRNA, | 8.17 | 2202 |
| 420234 | AW404908 | Hs.96038 | Ric (Drosophila)-like, expressed in many | 8.15 | 1399 |
| 447342 | AI199268 | Hs.19322 | Homo sapiens, Similar to RIKEN cDNA 2010 | 8.10 | 3845 |
| 423472 | AF041260 | Hs.129057 | breast carcinoma amplified sequence 1 | 8.08 | 1749 5181 |
| 414430 | AI346201 | Hs.76118 | ubiquitin carboxyl-terminal esterase L1 | 8.05 | 828 |
| 443715 | AI583187 | Hs.9700 | cyclin E1 | 8.05 | 3544 |
| 447033 | AI357412 | Hs.157601 | Predicted gene: Eos cloned; secreted w/V | 8.05 | 3814 |
| 407833 | AW955632 | Hs.66666 | ESTs, Weakly similar to S19560 proline-r | 8.05 | 204 |
| 447343 | AA256641 | Hs.236894 | ESTs, Highly similar to S02392 alpha-2-m | 8.04 | 3846 |
| 449349 | AI825386 | | hypothetical protein FLJ21939 similar to | 8.00 | 4057 |
| 420039 | NM_004605 | Hs.94581 | sulfotransferase family, cytosolic, 2B, | 7.97 | 1377 5061 |
| 429228 | AI553633 | | hypothetical protein MGC33630 | 7.94 | 2340 |
| 405770 | | | NM_002362: Homo sapiens melanoma antigen, | 7.85 | 4740 74 |
| 411605 | AW006831 | | ESTs | 7.82 | 563 |
| 418203 | X54942 | Hs.83758 | CDC28 protein kinase 2 | 7.80 | 1169 5003 |
| 442772 | AW503680 | Hs.5957 | Homo sapiens clone 24416 mRNA sequence | 7.80 | 3468 |
| 444783 | AK001468 | Hs.62180 | anillin (Drosophila Scraps homolog), act | 7.75 | 3628 5645 |
| 431374 | BE258532 | Hs.251871 | CTP synthase | 7.70 | 2551 |
| 414761 | AU077228 | Hs.77256 | enhancer of zeste (Drosophila) homolog 2 | 7.70 | 875 |
| 451752 | AB032997 | | KIAA1171 protein | 7.70 | 4247 5750 |
| 443171 | BE281128 | Hs.9030 | TONDU | 7.68 | 3501 |
| 444172 | BE147740 | | ESTs, Moderately similar I38022 hypot | 7.65 | 3580 |
| 445413 | AA151342 | Hs.12677 | CGI-147 protein | 7.65 | 3675 |
| 440700 | AW952281 | Hs.296184 | guanine nucleotide binding protein (G pr | 7.65 | 3313 |
| 407748 | AL079409 | Hs.38176 | KIAA0606 protein; SCN Circadian Oscillat | 7.60 | 188 |
| 423242 | AL039402 | Hs.125783 | DEME-6 protein | 7.58 | 1730 |
| 408633 | AW963372 | Hs.46677 | PRO2000 protein | 7.55 | 286 |
| 438461 | AW075485 | Hs.286049 | phosphoserine aminotransferase | 7.55 | 3141 |
| 424098 | AF077374 | Hs.139322 | Small proline-rich protein 3 | 7.55 | 1804 5199 |
| 432467 | T03667 | Hs.239388 | Human DNA sequence from clone RP1-304B14 | 7.54 | 2671 |
| 427581 | NM_014788 | Hs.179703 | KIAA0129 gene product | 7.50 | 2167 5339 |
| 428648 | AF052728 | Hs.188021 | potassium Voltage-gated channel, subfami | 7.50 | 2279 5369 |
| 425580 | L11144 | Hs.1907 | galanin | 7.40 | 1979 5272 |
| 400409 | AF153341 | | Homo sapiens winged helix/forkhead trans | 7.35 | 15 4687 |
| 414706 | AW340125 | Hs.76989 | KIAA0097 gene product | 7.35 | 865 |
| 429276 | AF056085 | Hs.198612 | G protein-coupled receptor 51 | 7.25 | 2346 5397 |
| 449845 | AW971183 | Hs.6019 | DnaJ (Hsp40) homolog, subfamily C, membe | 7.23 | 4088 |
| 410174 | AA306007 | Hs.59461 | DKFZP434C245 protein | 7.10 | 453 |
| 444670 | H58373 | Hs.332938 | hypothetical protein MGC5370 | 7.10 | 3618 |
| 418164 | AI761820 | Hs.41074 | ESTs, Weakly similar to I39294 McLeod sy | 7.05 | 1165 |
| 429597 | NM_003816 | Hs.2442 | a disintegrin and metalloproteinase doma | 7.00 | 2392 5412 |
| 429903 | AL134197 | Hs.93597 | cyclin-dependent kinase 5, regulatory su | 6.90 | 2425 |
| 428966 | AF059214 | Hs.194687 | cholesterol 25-hydroxylase | 6.90 | 2320 5385 |
| 424834 | AK001432 | Hs.153408 | Homo sapiens cDNA FLJ10570 fis, clone NT | 6.90 | 1889 |
| 430066 | AI929659 | Hs.237825 | signal recognition particle 72 kD | 6.88 | 2442 |
| 433212 | BE218049 | Hs.121820 | ESTs | 6.85 | 2749 |
| 420309 | AW043637 | Hs.21766 | ESTs, Weakly similar to ALU5_HUMAN ALU S | 6.85 | 1408 |
| 409557 | BE182896 | Hs.3686 | ESTs | 6.85 | 384 |
| 430294 | AI538226 | Hs.32976 | guanine nucleotide binding protein 4 | 6.83 | 2463 |
| 415539 | AI733881 | Hs.72472 | BMP-R1B (bone morphogenetic protein rec | 6.80 | 935 |
| 400292 | AA250737 | Hs.72472 | BMP-R1B (bone morphogenetic protein rec | 6.75 | 6 |
| 449048 | Z45051 | Hs.22920 | similar to S68401 (cattle) glucose induc | 6.75 | 4031 |
| 419743 | AW408762 | Hs.5957 | Homo sapiens clone 24416 mRNA sequence | 6.75 | 1356 |
| 430937 | X53463 | Hs.2704 | glutathione peroxidase 2 (gastrointestin | 6.75 | 2522 5466 |
| 442332 | AI693251 | Hs.8248 | Target CAT | 6.70 | 3421 |
| 441020 | W79283 | Hs.35962 | ESTs | 6.66 | 3325 |
| 428771 | AB028992 | Hs.193143 | KIAA1069 protein | 6.65 | 2295 5375 |
| 400205 | | | NM_006265*: Homo sapiens RAD21 (S. pombe) | 6.65 | 1 4680 |
| 438321 | AA576635 | Hs.6153 | CGI-48 protein | 6.65 | 3133 |
| 416208 | AW291168 | Hs.41295 | ESTs, Weakly similar to MUC2_HUMAN MUCIN | 6.65 | 981 |
| 421305 | BE397354 | Hs.324830 | diptheria toxin resistance protein requi | 6.65 | 1505 |
| 448275 | BE514434 | Hs.20830 | kinesin-like 2 | 6.62 | 3955 |
| 426283 | NM_003937 | Hs.169139 | kynureninase (L-kynurenine hydrolase) | 6.61 | 2048 5297 |
| 452099 | BE612992 | Hs.27931 | hypothetical protein FLJ10607 similar to | 6.60 | 4270 |
| 423134 | AJ012582 | Hs.124161 | hyperpolarization activated cyclic nucle | 6.57 | 1720 5170 |
| 408524 | D87942 | Hs.46328 | fucosyltransferase 2 (secretor status in | 6.55 | 275 4799 |
| 434094 | AA305599 | Hs.238205 | hypothetical protein PRO2013 | 6.50 | 2814 |
| 429183 | AB014604 | Hs.197955 | KIAA0704 protein | 6.50 | 2337 5393 |
| 426559 | AB001914 | Hs.170414 | paired basic amino acid cleaving system | 6.50 | 2078 5309 |
| 418327 | U70370 | Hs.84136 | paired-like homeodomain transcription fa | 6.50 | 1185 5009 |
| 407999 | AI126271 | Hs.49433 | ESTs, Weakly similar to YZ28_HUMAN HYPOT | 6.48 | 222 |
| 458814 | AI498957 | Hs.170861 | ESTs, Weakly similar to Z195_HUMAN ZINC | 6.45 | 4638 |
| 438209 | AL120659 | Hs.6111 | aryl-hydrocarbon receptor nuclear transl | 6.45 | 3126 |
| 423634 | AW959908 | Hs.1690 | heparin-binding growth factor binding pr | 6.45 | 1762 |
| 430300 | U60805 | Hs.238648 | oncostatin M receptor | 6.40 | 2465 5441 |
| 426827 | AW067805 | Hs.172665 | methylenetetrahydrofolate dehydrogenase | 6.39 | 2104 |
| 401197 | | | ENSP00000229263*: HSPC213. | 6.39 | |
| 408771 | AW732573 | Hs.47584 | potassium voltage-gated channel, delayed | 6.35 | 305 |
| 432731 | R31178 | Hs.287820 | fibronectin 1 | 6.35 | 2695 |
| 439979 | AW600291 | Hs.6823 | hypothetical protein FLJ10430 | 6.33 | 3251 |
| 441362 | BE614410 | Hs.23044 | RAD51 (S. cerevisiae) homolog (E. coli Re | 6.31 | 3347 |
| 413450 | Z99716 | Hs.75372 | N-acetylgalactosaminidase, alpha- | 6.30 | 4901 723 |
| 410762 | AF226053 | Hs.66170 | HSKM-B protein | 6.30 | 4857 514 |
| 434263 | N34895 | Hs.79187 | ESTs | 6.30 | 2825 |
| 412715 | NM_000947 | Hs.74519 | primase, polypeptide 2A (58 kD) | 6.29 | 4885 662 |
| 433556 | W56321 | Hs.111460 | calcium/calmodulin-dependent protein kin | 6.25 | 2768 |
| 419631 | AW188117 | | popeye protein 3 | 6.25 | 1340 |
| 407235 | D20569 | Hs.169407 | SAC2 (suppressor of actin mutations 2, y | 6.25 | 140 |
| 428484 | AF104032 | Hs.184601 | solute carrier family 7 (cationic amino | 6.23 | 2265 5364 |
| 408452 | AA054683 | Hs.192455 | ESTs, Weakly similar to ALU7_HUMAN ALU S | 6.22 | 268 |
| 412942 | AL120344 | Hs.75074 | mitogen-activated protein kinase-activat | 6.21 | 685 |
| 447397 | BE247676 | Hs.18442 | E-1 enzyme | 6.20 | 3856 |
| 400297 | AI127076 | Hs.306201 | hypothetical protein DKFZp564O1278 | 6.20 | 9 |
| 452259 | AA317439 | Hs.28707 | signal sequence receptor, gamma (translo | 6.20 | 4290 |
| 446636 | AC002563 | Hs.15767 | citron (rho-interacting, serine/threonin | 6.19 | 3773 5674 |
| 450800 | BE395161 | Hs.1390 | proteasome (prosome, macropain) subunit, | 6.19 | 4167 |
| 408527 | AL135018 | Hs.33074 | Homo sapiens, clone IMAGE: 3606519, mRNA, | 6.17 | 276 |
| 416426 | AA180256 | Hs.210473 | Homo sapiens cDNA FLJ14872 fis, clone PL | 6.15 | 996 |
| 424544 | M88700 | Hs.150403 | dopa decarboxylase (aromatic L-amino aci | 6.15 | 1858 5217 |
| 426471 | M22440 | Hs.170009 | transforming growth factor, alpha | 6.15 | 2068 5305 |
| 428004 | AA449563 | Hs.151393 | glutamate-cysteine ligase, catalytic sub | 6.15 | 2213 |
| 452240 | AI591147 | Hs.61232 | ESTs | 6.15 | 4286 |
| 417933 | X02308 | Hs.82962 | thymidylate synthetase | 6.12 | 1139 4996 |
| 434826 | AF155661 | Hs.22265 | pyruvate dehydrogenase phosphatase | 6.11 | 2871 5556 |
| 423198 | M81933 | Hs.1634 | cell division cycle 25A | 6.10 | 1727 5174 |
| 416294 | D86980 | Hs.79170 | KIAA0227 protein | 6.10 | 4958 984 |
| 429663 | M68874 | Hs.211587 | phospholipase A2, group IVA (cytosolic, | 6.08 | 2404 5422 |
| 407944 | R34008 | Hs.239727 | desmocollin 2 | 6.08 | 218 |
| 422283 | AW411307 | Hs.114311 | CDC45 (cell division cycle 45, S. cerevis | 6.07 | 1626 |
| 451027 | AW519204 | Hs.40808 | Homo sapiens, Similar to RIKEN cDNA 2810 | 6.06 | 4183 |
| 401519 | | | C15000476*: gi\|12737279\|ref\|XP_012163.1\| | 6.05 | |
| 420281 | AI623693 | Hs.323494 | Predicted cation efflux pump | 6.05 | 1405 |
| 408908 | BE296227 | Hs.250822 | serine/threonine kinase 15 | 6.05 | 317 |
| 415138 | C18356 | Hs.295944 | tissue factor pathway inhibitor 2 | 6.00 | 910 |
| 420153 | N22120 | Hs.75277 | hypothetical protein FLJ13910 | 5.96 | 1389 |
| 433437 | U20536 | Hs.3280 | caspase 6, apoptosis-related cysteine pr | 5.96 | 2762 5535 |
| 408045 | AW138959 | Hs.245123 | ESTs | 5.95 | 227 |
| 433675 | AW977653 | Hs.75319 | ribonucleotide reductase M2 polypeptide | 5.95 | 2779 |
| 446595 | T57448 | Hs.15467 | hypothetical protein FLJ20725 | 5.95 | 3768 |
| 408298 | AI745325 | Hs.271923 | Homo sapiens cDNA: FLJ22785 fis, clone K | 5.95 | 253 |
| 417454 | NM_000202 | Hs.172458 | iduronate 2-sulfatase (Hunter syndrome) | 5.94 | 1097 4986 |
| 425071 | NM_013989 | Hs.154424 | deiodinase, iodothyronine, type II | 5.91 | 1916 5244 |
| 416933 | BE561850 | Hs.80506 | small nuclear ribonucleoprotein polypept | 5.90 | 1040 |
| 457579 | AB030816 | Hs.36761 | HRAS-like suppressor | 5.90 | 4595 5813 |
| 428987 | NM_004751 | Hs.194710 | glucosaminyl (N-acetyl) transferase 3, m | 5.90 | 2324 5386 |
| 406399 | | | NM_003122*: Homo sapiens serine protease | 5.90 | 4743 78 |
| 442739 | NM_007274 | Hs.8679 | cytosolic acyl coenzyme A thioester hydr | 5.87 | 3464 5626 |
| 420344 | BE463721 | Hs.97101 | putative G protein-coupled receptor | 5.85 | 1415 |
| 452092 | BE245374 | Hs.27842 | hypothetical protein FLJ11210 | 5.85 | 4268 |
| 429925 | NM_000786 | Hs.226213 | cytochrome P450, 51 (lanosterol 14-alpha | 5.85 | 2428 |
| 453204 | R10799 | Hs.191990 | ESTs | 5.85 | 4385 |
| 433345 | AI681545 | Hs.152982 | hypothetical protein FLJ13117 | 5.85 | 2757 |
| 431548 | AI834273 | Hs.9711 | novel protein | 5.85 | 2564 |
| 429966 | BE081342 | Hs.283037 | HSPC039 protein | 5.80 | 2431 |
| 414341 | D80004 | Hs.75909 | KIAA0182 protein | 5.80 | 4919 813 |
| 449145 | AI632122 | Hs.198408 | ESTs | 5.80 | 4039 |
| 447334 | AA515032 | Hs.91109 | ESTs | 5.80 | 3844 |
| 423453 | AW450737 | Hs.128791 | CGI-09 protein | 5.80 | 1748 |
| 410619 | BE512730 | Hs.65114 | keratin 18 | 5.74 | 498 |
| 416990 | AF124145 | Hs.80731 | autocrine motility factor receptor | 5.73 | 1049 4975 |
| 430454 | AW469011 | Hs.105635 | ESTs | 5.72 | 2487 |
| 430569 | AF241254 | Hs.178098 | angiotensin I converting enzyme (peptidy | 5.72 | 2496 5456 |
| 419926 | AW900992 | Hs.93796 | DKFZP586D2223 protein | 5.70 | 1371 |
| 443180 | R15875 | Hs.258576 | claudin 12 | 5.70 | 3504 |
| 428832 | AA578229 | Hs.324239 | ESTs, Moderately similar to ZN91_HUMAN Z | 5.70 | 2308 |
| 433001 | AF217513 | Hs.279905 | clone HQ0310 PRO0310p1 | 5.67 | 2719 5521 |
| 425397 | J04088 | Hs.156346 | topoisomerase (DNA) II alpha (170 KD) | 5.67 | 1959 5261 |
| 443426 | AF098158 | Hs.9329 | chromosome 20 open reading frame 1 | 5.65 | 3523 5627 |
| 409619 | AK001015 | Hs.55220 | BCL2-associated athanogene 2 | 5.65 | 388 |
| 416815 | U41514 | Hs.80120 | UDP-N-acetyl-alpha-D-galactosamine: polyp | 5.65 | 1027 4968 |
| 407378 | AA299264 | Hs.57776 | ESTs, Moderately similar to I38022 hypot | 5.65 | 160 |
| 427658 | H61387 | Hs.30868 | nogo receptor | 5.65 | 2175 |
| 445903 | AI347487 | Hs.132781 | class I cytokine receptor | 5.65 | 3711 |
| 401866 | | | Target Exon | 5.65 | |
| 439680 | AW245741 | Hs.58461 | ESTs, Weakly similar to A35659 krueppel- | 5.65 | 3229 |
| 415752 | BE314524 | Hs.78776 | putative transmembrane protein | 5.64 | 948 |
| 440351 | AF030933 | Hs.7179 | RAD1 (S. pombe) homolog | 5.60 | 3285 5607 |
| 434834 | AF156774 | Hs.324020 | 1-acylglycerol-3-phosphate O-acyltransfe | 5.60 | 2872 5557 |
| 452461 | N78223 | Hs.108106 | transcription factor | 5.60 | 4311 |
| 424140 | Z48051 | Hs.141308 | myelin oligodendrocyte glycoprotein | 5.60 | 1809 5200 |
| 409439 | AW390511 | Hs.288862 | Homo sapiens cDNA: FLJ21260 fis, clone C | 5.59 | 371 |
| 442315 | AA173992 | Hs.7956 | ESTs, Moderately similar to ZN91_HUMAN Z | 5.58 | 3414 |
| 437389 | AL359587 | Hs.271586 | hypothetical protein DKFZp762M115 | 5.55 | 3063 5586 |
| 400277 | | | Eos Control | 5.52 | |
| 442313 | BE388898 | Hs.8215 | hypothetical protein FLJ11307 | 5.50 | 3413 |
| 449444 | AW818436 | | solute carrier family 16 (monocarboxylic | 5.50 | 4062 |
| 420734 | AW972872 | Hs.293736 | ESTs | 5.50 | 1447 |
| 422756 | AA441787 | Hs.119689 | glycoprotein hormones, alpha polypeptide | 5.45 | 1679 |
| 442961 | BE614474 | | F-box only protein 22 | 5.45 | 3484 |
| 439580 | AF086401 | Hs.293847 | ESTs, Moderately similar to S65657 alpha | 5.45 | 3220 |
| 439772 | AL365406 | Hs.10268 | Homo sapiens mRNA full length insert cDN | 5.45 | 3234 |
| 443695 | AW204099 | | ESTs, Weakly similar to AF126780 1 retin | 5.45 | 3541 |
| 409757 | NM_001898 | Hs.123114 | cystatin SN | 5.43 | 403 4832 |
| 422150 | AI867118 | | calpastatin | 5.42 | 1609 |
| 450325 | AI935962 | Hs.91973 | ESTs | 5.41 | 4129 |
| 444758 | AL044878 | Hs.11899 | 3-hydroxy-3-methylglutaryl-Coenzyme A re | 5.40 | 3625 |
| 445266 | BE222118 | Hs.12479 | associated molecule with the SH3 domain | 5.40 | 3665 |
| 457001 | J03258 | Hs.2062 | vitamin D (1,25-dihydroxyvitamin D3) re | 5.40 | 4574 5808 |
| 422938 | NM_001809 | Hs.1594 | centromere protein A (17 kD) | 5.40 | 1694 5162 |
| 446342 | BE298665 | Hs.14846 | solute carrier family 7 (cationic amino | 5.40 | 3746 |
| 425994 | AK000207 | Hs.165803 | hypothetical protein FLJ20200 | 5.37 | 2017 5285 |
| 409799 | D11928 | Hs.76845 | phosphoserine phosphatase-like | 5.35 | 407 |
| 437834 | AA769294 | | gb: nz36g03.s1 NCI_CGAP_GCB1 Homo sapiens | 5.35 | 3096 |
| 407244 | M10014 | | fibrinogen, gamma polypeptide | 5.35 | 143 4767 |
| 437762 | T78028 | Hs.154679 | synaptotagmin I | 5.33 | 3088 |
| 441633 | AW958544 | Hs.112242 | normal mucosa of esophagus specific 1 | 5.32 | 3363 |
| 452223 | AA425467 | Hs.8035 | hypothetical protein MGC2827 | 5.31 | 4283 |
| 450256 | AA286887 | Hs.24724 | MFH-amplified sequences with leucine-ric | 5.31 | 4120 |
| 430335 | D80007 | Hs.239499 | KIAA0185 protein | 5.30 | 2473 5448 |
| 430287 | AW182459 | Hs.125759 | ESTs, Weakly similar to LEU5_HUMAN LEUKE | 5.30 | 2460 |
| 433485 | AI493076 | Hs.306098 | aldo-keto reductase family 1, member C2 | 5.30 | 2766 |
| 423739 | AA398155 | Hs.97600 | ESTs | 5.30 | 1771 |
| 458621 | AI221741 | Hs.117777 | ESTs | 5.30 | 4630 |
| 413585 | AI133452 | Hs.75431 | fibrinogen, gamma polypeptide | 5.30 | 737 |
| 427719 | AI393122 | Hs.134726 | ESTs | 5.29 | 2189 |
| 452721 | AJ269529 | Hs.301871 | solute carrier family 37 (glycerol-3-pho | 5.27 | 4337 5761 |
| 445139 | AB037848 | Hs.12365 | synaptotagmin XIII | 5.27 | 3656 5652 |
| 422095 | AI868872 | Hs.282804 | hypothetical protein FLJ22704 | 5.26 | 1603 |
| 438915 | AA280174 | Hs.285681 | Williams-Beuren syndrome chromosome regi | 5.25 | 3178 |
| 425322 | U63630 | Hs.155637 | protein kinase, DNA-activated, catalytic | 5.24 | 1950 5255 |
| 437915 | AI637993 | Hs.202312 | Homo sapiens clone N11 NTera2D1 teratoca | 5.20 | 3105 |
| 444735 | BE019923 | Hs.243122 | hypothetical protein FLJ13057 similar to | 5.20 | 3623 |
| 439574 | AI469788 | | ESTs | 5.20 | 3219 |
| 408411 | C15118 | Hs.322482 | hypothetical protein DKFZp566J2046 | 5.20 | 265 |
| 421077 | AK000061 | Hs.101590 | hypothetical protein | 5.20 | 1479 5093 |
| 437659 | AB007944 | Hs.5737 | KIAA0475 gene product | 5.15 | 3079 5589 |
| 433023 | AW864793 | | thrombospondin 1 | 5.15 | 2725 |
| 417059 | AL037672 | Hs.81071 | extracellular matrix protein 1 | 5.14 | 1059 |
| 434061 | AW024973 | Hs.283675 | NPD009 protein | 5.12 | 2810 |
| 431448 | AL137517 | Hs.306201 | hypothetical protein DKFZp564O1278 | 5.10 | 2555 5476 |
| 404568 | | | NM_022071*: Homo sapiens hypothetical pro | 5.10 | 4723 56 |
| 420942 | H03514 | Hs.15589 | ESTs | 5.10 | 1467 |
| 410340 | AW182833 | Hs.112188 | hypothetical protein FLJ13149 | 5.10 | 466 |
| 441031 | AI110684 | Hs.7645 | fibrinogen, B beta polypeptide | 5.10 | 3329 |
| 433862 | D86960 | Hs.3610 | KIAA0205 gene product | 5.10 | 2793 5542 |
| 439108 | AW163034 | Hs.6467 | synaptogyrin 3 | 5.07 | 3186 |
| 435664 | AI032087 | Hs.269819 | ESTs | 5.05 | 2936 |
| 422486 | BE514492 | Hs.117487 | gene near HD on 4p16.3 with homology to | 5.05 | 1648 |
| 431789 | H19500 | Hs.269222 | mitogen-activated protein kinase 4 | 5.05 | 2599 |
| 424905 | NM_002497 | Hs.153704 | NIMA (never in mitosis gene a)-related k | 5.05 | 1898 5237 |
| 442053 | R35343 | Hs.24968 | Human DNA sequence from clone RP1-233G16 | 5.05 | 3391 |
| 415691 | AW963979 | Hs.24723 | ESTs | 5.00 | 944 |
| 408705 | AA312135 | Hs.46967 | HSPCO34 protein | 5.00 | 295 |
| 424179 | F30712 | Hs.334573 | Homo sapiens, clone IMAGE: 4285740, mRNA | 5.00 | 1812 |
| 426318 | AA375125 | Hs.147112 | Homo sapiens cDNA: FLJ22322 fis, clone H | 5.00 | 2051 |
| 427675 | AW138190 | Hs.180248 | zinc finger protein 124 (HZF-16) | 5.00 | 2178 |
| 407198 | H91679 | | gb: yv04a07.s1 Soares fetal liver spleen | 5.00 | 135 |
| 412935 | BE267045 | Hs.75064 | tubulin-specific chaperone c | 4.97 | 683 |
| 432378 | AI493046 | Hs.146133 | ESTs | 4.95 | 2662 |
| 428728 | NM_016625 | Hs.191381 | hypothetical protein | 4.95 | 2288 5373 |
| 416111 | AA033813 | Hs.79018 | chromatin assembly factor 1, subunit A ( | 4.95 | 972 |
| 441224 | AU076964 | Hs.7753 | calumenin | 4.95 | 3338 |
| 415339 | NM_015156 | Hs.78398 | KIAA0071 protein | 4.95 | 4946 927 |
| 450353 | AI244661 | Hs.103296 | ESTs, Weakly similar to S65657 alpha-1C- | 4.95 | 4132 |
| 441377 | BE218239 | Hs.202656 | ESTs | 4.95 | 3349 |
| 422765 | AW409701 | Hs.1578 | baculoviral IAP repeat-containing 5 (sur | 4.95 | 1680 |
| 431566 | AF176012 | Hs.260720 | J domain containing protein 1 | 4.94 | 2568 5479 |
| 429788 | U87791 | Hs.221040 | HBS1 (S. cerevisiae)-like | 4.94 | 2417 5426 |
| 404561 | | | trichorhinophalangeal syndrome I gene (T | 4.93 | |
| 437739 | AW579216 | Hs.264610 | ESTs, Moderately similar to lbd1 [H. sapi | 4.93 | 3084 |
| 438549 | BE386801 | Hs.21858 | trinucleotide repeat containing 3 | 4.92 | 3147 |
| 424308 | AW975531 | Hs.154443 | minichromosome maintenance deficient (S. | 4.91 | 1827 |
| 403485 | | | C3001813*: gi\|12737279\|ref\|XP_012163.1\|k | 4.90 | |
| 433929 | AI375499 | Hs.27379 | ESTs | 4.90 | 2799 |
| 416000 | R82342 | Hs.79856 | ESTs, Weakly similar to S65657 alpha-1C- | 4.90 | 965 |
| 424381 | AA285249 | Hs.146329 | protein kinase Chk2 (CHEK2) | 4.90 | 1838 |
| 409420 | Z15008 | Hs.54451 | laminin, gamma 2 (nicein (100 kD), kalini | 4.89 | 368 4824 |
| 426006 | R49031 | Hs.22627 | ESTs | 4.86 | 2019 |
| 447349 | AI375546 | | gb: tc23d04.x1 Soares_total_fetus_Nb2HF8_{—} | 4.86 | 3848 |
| 415786 | AW419196 | Hs.257924 | hypothetical protein FLJ13782 | 4.85 | 951 |
| 450149 | AW969781 | Hs.132863 | Zic family member 2 (odd-paired Drosophi | 4.85 | 4106 |
| 423551 | AA327598 | Hs.89633 | ESTs | 4.83 | 1757 |
| 417348 | AI940507 | Hs.318526 | hypothetical protein FLJ12661 | 4.81 | 1084 |
| 422616 | BE300330 | Hs.118725 | selenophosphate synthetase 2 | 4.80 | 1663 |
| 427209 | H06509 | Hs.92423 | KIAA1566 protein | 4.80 | 2132 |
| 423554 | M90516 | Hs.1674 | glutamine-fructose-6-phosphate transamin | 4.80 | 1758 5182 |
| 419586 | AI088485 | Hs.144759 | ESTs, Weakly similar to I38022 hypotheti | 4.80 | 1335 |
| 437435 | AA249439 | Hs.27027 | hypothetical protein DKFZp762H1311 | 4.80 | 3065 |
| 424602 | AK002055 | Hs.151046 | hypothetical protein FLJ11193 | 4.80 | 1863 5219 |
| 442760 | BE075297 | Hs.6614 | ESTs, Weakly similar to A43932 mucin 2 p | 4.80 | 3466 |
| 458098 | BE550224 | | metallothionein 1E (functional) | 4.80 | 4611 |
| 418322 | AA284166 | Hs.84113 | cyclin-dependent kinase inhibitor 3 (CDK | 4.77 | 1184 |
| 420956 | AA351584 | Hs.100543 | Homo sapiens clone 24505 mRNA sequence | 4.76 | 1468 |
| 408296 | AL117452 | Hs.44155 | DKFZP586G1517 protein | 4.75 | 252 4792 |
| 414343 | AL036166 | Hs.75914 | coated vesicle membrane protein | 4.75 | 814 |
| 438613 | C05569 | Hs.243122 | hypothetical protein FLJ13057 similar to | 4.75 | 3153 |
| 458809 | AW972512 | Hs.20985 | sin3-associated polypeptide, 30 kD | 4.75 | 4637 |
| 430935 | AW072916 | | zinc finger protein 131 (clone pHZ-10) | 4.75 | 2521 |
| 441285 | NM_002374 | Hs.167 | microtubule-associated protein 2 | 4.75 | 3343 5616 |
| 433201 | AB040896 | Hs.21104 | KIAA1463 protein | 4.75 | 2747 5532 |
| 441553 | AA281219 | Hs.121296 | ESTs | 4.74 | 3357 |
| 402812 | | | NM_004930*: Homo sapiens capping protein | 4.73 | 39 4708 |
| 434203 | BE262677 | Hs.283558 | hypothetical protein PRO1855 | 4.72 | 2820 |
| 431899 | AA521381 | Hs.187726 | ESTs | 4.71 | 2614 |
| 401464 | | | histone deacetylase 5 | 4.70 | |
| 413431 | AW246428 | Hs.75355 | ubiquitin-conjugating enzyme E2N (homolo | 4.70 | 720 |
| 453496 | AA442103 | Hs.33084 | solute carrier family 2 (facilitated glu | 4.70 | 4413 |
| 447276 | AL049795 | Hs.17987 | hypothetical protein MGC1203 | 4.70 | 3838 |
| 415927 | AL120168 | Hs.78919 | Kell blood group precursor (McLeod pheno | 4.70 | 961 |
| 418250 | U29926 | Hs.83918 | adenosine monophosphate deaminase (isofo | 4.70 | 1173 5006 |
| 428654 | NM_012091 | Hs.188661 | adenosine deaminase, tRNA-specific 1 | 4.70 | 2280 5370 |
| 412537 | AL031778 | | nuclear transcription factor Y, alpha | 4.70 | 644 |
| 421898 | AA259011 | Hs.109268 | hypothetical protein FLJ12552 | 4.70 | 1578 |
| 425523 | AB007948 | Hs.158244 | KIAA0479 protein | 4.70 | 1973 5268 |
| 451061 | AW291487 | Hs.213659 | ESTs, Weakly similar to KIAA1357 protein | 4.70 | 4186 |
| 424378 | W28020 | Hs.167988 | neural cell adhesion molecule 1 | 4.68 | 1837 |
| 426997 | BE620738 | Hs.173125 | peptidylprolyl isomerase F (cyclophilin | 4.67 | 2120 |
| 441024 | AW081530 | Hs.268231 | ESTs | 4.65 | 3327 |
| 424085 | NM_002914 | Hs.139226 | replication factor C (activator 1) 2 (40 | 4.65 | 1803 5198 |
| 432840 | AK001403 | Hs.279521 | hypothetical protein FLJ20530 | 4.65 | 2704 5516 |
| 428829 | R14050 | Hs.194051 | Homo sapiens mRNA; cDNA DKFZp566B213 (fr | 4.65 | 2306 |
| 438898 | AI819863 | Hs.106243 | ESTs | 4.65 | 3177 |
| 426784 | U03749 | Hs.172216 | chromogranin A (parathyroid secretory pr | 4.65 | 2098 5319 |
| 435937 | AA830893 | Hs.119769 | ESTs | 4.65 | 2953 |
| 419551 | AW582256 | Hs.91011 | anterior gradient 2 (Xenepus laevis) hom | 4.63 | 1330 |
| 431512 | BE270734 | Hs.2795 | lactate dehydrogenase A | 4.62 | 2561 |
| 452834 | AI638627 | Hs.105685 | KIAA1688 protein | 4.60 | 4352 |
| 444858 | AI199738 | Hs.208275 | ESTs, Weakly similar to ALUA_HUMAN !!!! | 4.60 | 3633 |
| 447519 | U46258 | Hs.339665 | ESTs | 4.60 | 3873 |
| 408089 | H59799 | Hs.42644 | thioredoxin-like | 4.60 | 233 |
| 413597 | AW302885 | Hs.117183 | ESTs | 4.60 | 739 |
| 449543 | AF070632 | Hs.23729 | Homo sapiens clone 24405 mRNA sequence | 4.60 | 4066 |
| 418803 | U50079 | Hs.88556 | histone deacetylase 1 | 4.59 | 1241 5029 |
| 408363 | NM_003389 | Hs.44396 | coronin, actin-binding protein, 2A | 4.58 | 260 4794 |
| 432441 | AW292425 | Hs.163484 | intron of hepatocyte nuclear factor-3 al | 4.55 | 2668 |
| 414770 | BE257224 | | Homo sapiens, clone IMAGE: 3873720, mRNA | 4.55 | 876 |
| 433561 | BE540937 | Hs.20104 | hypothetical protein FLJ00052 | 4.55 | 2769 |
| 412970 | AB026436 | Hs.177534 | dual specificity phosphatase 10 | 4.55 | 4890 687 |
| 421928 | AF013758 | Hs.109643 | polyadenylate binding protein-interactin | 4.55 | 1580 5129 |
| 413476 | U25849 | Hs.75393 | acid phosphatase 1, soluble | 4.54 | 4902 726 |
| 426158 | NM_001982 | Hs.199067 | v-erb-b2 avian erythroblastic leukemia v | 4.53 | 2033 5294 |
| 406181 | | | Target Exon | 4.52 | |
| 449644 | AW960707 | Hs.148324 | ESTs | 4.52 | 4072 |
| 438714 | AA814859 | | ESTs | 4.50 | 3161 |
| 438880 | AF075022 | Hs.101480 | ESTs | 4.50 | 3174 |
| 431452 | AI073641 | Hs.152372 | ESTs, Weakly similar to 2109260A B cell | 4.50 | 2556 |
| 442108 | AW452649 | Hs.166314 | ESTs | 4.50 | 3398 |
| 416072 | AL110370 | Hs.79000 | growth associated protein 43 | 4.49 | 970 |
| 414320 | U13616 | Hs.75893 | ankyrin 3, node of Ranvier (ankyrin G) | 4.49 | 4917 810 |
| 446572 | AV659151 | Hs.282961 | ESTs | 4.48 | 3765 |
| 420005 | AW271106 | Hs.133294 | ESTs | 4.47 | 1372 |
| 423979 | AF229181 | Hs.136644 | CS box-containing WD protein | 4.45 | 1786 5191 |
| 425849 | AJ000512 | Hs.296323 | serum/glucocorticoid regulated kinase | 4.45 | 2005 5279 |
| 443257 | AI334040 | Hs.11614 | HSPC065 protein | 4.45 | 3514 |
| 421227 | R78581 | Hs.266308 | mosaic serine protease | 4.45 | 1492 |
| 432339 | AW411259 | | ESTs | 4.45 | 2656 |
| 426699 | AA383337 | Hs.121269 | ESTs | 4.45 | 2089 |
| 418661 | NM_001949 | Hs.1189 | E2F transcription factor 3 | 4.45 | 1222 5022 |
| 439609 | AW971945 | Hs.293236 | ESTs | 4.45 | 3225 |
| 438869 | AF075009 | | gb: Homo sapiens full length insert cDNA | 4.45 | 3171 |
| 424163 | NM_007071 | Hs.142245 | HERV-H LTR-associating 3 | 4.43 | 1811 5201 |
| 452459 | AI356895 | Hs.49359 | hypothetical protein DKFZp547E052 | 4.43 | 4310 |
| 408548 | AA055449 | Hs.63187 | ESTs, Weakly similar to ALUC_HUMAN !!!! | 4.40 | 278 |
| 419594 | AA013051 | Hs.91417 | topoisomerase (DNA) II binding protein | 4.40 | 1339 |
| 431912 | AI660552 | | ESTs, Weakly similar to A56154 AbI subst | 4.40 | 2615 |
| 407930 | AA045847 | Hs.188361 | Homo sapiens cDNA FLJ12807 fis, clone NT | 4.40 | 215 |
| 416902 | AA375634 | Hs.288974 | hypothetical protein FLJ12528 | 4.40 | 1035 |
| 423673 | BE003054 | Hs.1695 | matrix metalloproteinase 12 (macrophage | 4.40 | 1765 |
| 429687 | AI675749 | Hs.211608 | nucleoporin 153 kD | 4.40 | 2406 |
| 404996 | | | Target Exon | 4.40 | |
| 415156 | X84908 | Hs.78060 | phosphorylase kinase, beta | 4.39 | 4940 911 |
| 445871 | AI702901 | Hs.145582 | ESTs, Weakly similar to FOR4 MOUSE FORMI | 4.38 | 3706 |
| 420522 | AW957137 | Hs.98541 | hypothetical protein | 4.38 | 1427 |
| 418816 | T29621 | Hs.88778 | carbonyl reductase 1 | 4.37 | 1243 |
| 457650 | AA649162 | Hs.236456 | ESTs | 4.36 | 4596 |
| 422809 | AK001379 | Hs.121028 | hypothetical protein FLJ10549 | 4.35 | 1684 |
| 421437 | AW821252 | Hs.104336 | hypothetical protein | 4.35 | 1519 |
| 450251 | BE080483 | | gb: QV1-BT0630-280200-086-a05 BT0630 Homo | 4.35 | 4118 |
| 436869 | NM_014867 | Hs.5333 | KIAA0711 gene product | 4.35 | 3015 5581 |
| 448552 | AW973653 | Hs.20104 | hypothetical protein FLJ00052 | 4.35 | 3983 |
| 427083 | NM_006363 | Hs.173497 | Sec23 (S. cerevisiae) homolog B | 4.32 | 2124 5330 |
| 445160 | AI299144 | Hs.101937 | sine oculis homeobox (Drosophila) homolo | 4.32 | 3657 |
| 423681 | AB023215 | Hs.131525 | Homo sapiens mRNA; cDNA DKFZp434E199 (fr | 4.31 | 1766 5184 |
| 412452 | AA215731 | | suppression of tumorigenicity 5 | 4.31 | 634 |
| 433020 | AI375726 | Hs.279918 | hypothetical protein | 4.30 | 2724 |
| 434815 | AF155582 | Hs.46744 | core1 UDP-galactose: N-acetylgalactosamin | 4.30 | 2870 5555 |
| 402802 | | | NM_001397*: Homo sapiens endothelin conver | 4.30 | 38 4707 |
| 402408 | | | NM_030920: Homo sapiens hypothetical pro | 4.30 | 33 4703 |
| 445867 | AF272663 | Hs.13405 | gephyrin | 4.30 | 3705 5660 |
| 448920 | AW408009 | Hs.22580 | alkylglycerone phosphate synthase | 4.30 | 4020 |
| 446006 | NM_004403 | Hs.13530 | deafness, autosomal dominant 5 | 4.28 | 3717 5662 |
| 430589 | AJ002744 | Hs.246315 | UDP-N-acetyl-alpha-D-galactosamine: polyp | 4.27 | 2497 5457 |
| 407807 | AL031427 | Hs.40094 | Human DNA sequence from clone 167A19 on | 4.27 | 198 |
| 417615 | BE548641 | Hs.82314 | hypoxanthine phosphoribosyltransferase 1 | 4.27 | 1107 |
| 425843 | BE313280 | Hs.159627 | death associated protein 3 | 4.26 | 2003 |
| 443837 | AI984625 | Hs.9884 | spindle pole body protein | 4.26 | 3559 |
| 425387 | AB037864 | Hs.156051 | KIAA1443 protein | 4.26 | 1958 5260 |
| 432978 | AF126743 | Hs.279884 | DNAJ domain-containing | 4.26 | 2717 5520 |
| 431797 | BE169641 | Hs.270134 | hypothetical protein FLJ20280 | 4.26 | 2601 |
| 410366 | AI267589 | Hs.302689 | hypothetical protein | 4.26 | 469 |
| 432350 | NM_005865 | Hs.274407 | protease, serine, 16 (thymus) | 4.25 | 2658 5507 |
| 433002 | AF048730 | Hs.279906 | cyclin T1 | 4.25 | 2720 5522 |
| 422168 | AA586894 | Hs.112408 | S100 calcium-binding protein A7 (psorias | 4.25 | 1612 |
| 453005 | AW055308 | Hs.31803 | ESTs, Weakly similar to N-WASP [H. sapien | 4.23 | 4365 |
| 421875 | AA299607 | Hs.98969 | ESTs | 4.21 | 1574 |
| 432140 | AK000404 | Hs.272688 | hypothetical protein FLJ20397 | 4.20 | 2634 5500 |
| 402053 | | | C11001722*: gi\|11436283\|ref\|XP_006959.1\| | 4.20 | |
| 440426 | AI159800 | Hs.7181 | Homo sapiens cDNA FLJ13663 fis, clone PL | 4.20 | 3287 |
| 428479 | Y00272 | Hs.334562 | cell division cycle 2, G1 to S and G2 to | 4.20 | 2264 5363 |
| 418259 | AA215404 | | ESTs | 4.20 | 1174 |
| 413582 | AW295647 | Hs.71331 | hypothetical protein MGC5350 | 4.20 | 736 |
| 436679 | AI127483 | Hs.120451 | ESTs, Weakly similar to unnamed protein | 4.20 | 3002 |
| 446880 | AI811807 | Hs.108646 | Homo sapiens cDNA FLJ14934 fis, clone PL | 4.19 | 3799 |
| 449976 | H06350 | Hs.135056 | Human DNA sequence from clone RP5-850E9 | 4.19 | 4095 |
| 433409 | AI278802 | Hs.25661 | ESTs | 4.19 | 2761 |
| 451380 | H09280 | Hs.13234 | ESTs | 4.16 | 4218 |
| 434540 | NM_016045 | Hs.3945 | CGI-107 protein | 4.16 | 2847 5549 |
| 456844 | AI264155 | Hs.152981 | CDP-diacylglycerol synthase (phosphatida | 4.16 | 4562 |
| 409445 | AW341217 | Hs.14139 | ESTs, Weakly similar to JC5314 CDC28/cdc | 4.15 | 373 |
| 448999 | AF179274 | Hs.22791 | transmembrane protein with EGF-like and | 4.15 | 4024 5717 |
| 452110 | T47667 | Hs.28005 | Homo sapiens cDNA FLJ11309 fis, clone PL | 4.15 | 4272 |
| 437682 | AA476652 | Hs.94952 | Homo sapiens cDNA: FLJ23371 fis, clone H | 4.15 | 3083 |
| 437250 | BE257342 | Hs.94576 | hypothetical protein MGC3062 | 4.15 | 3048 |
| 452279 | AA286844 | | hypothetical protein FLJ13164 | 4.15 | 4293 |
| 425242 | D13635 | Hs.155287 | KIAA0010 gene product | 4.15 | 1942 |
| 433388 | AI432672 | Hs.288539 | hypothetical protein FLJ22191 | 4.15 | 2759 |
| 448072 | AI459306 | Hs.24908 | ESTs | 4.15 | 3940 |
| 423073 | BE252922 | Hs.123119 | MAD (mothers against decapentaplegic, Dr | 4.15 | 1715 |
| 444371 | BE540274 | Hs.239 | forkhead box M1 | 4.14 | 3592 |
| 453951 | AI676235 | Hs.32163 | ESTs | 4.10 | 4455 |
| 422576 | BE548555 | Hs.118554 | CGI-83 protein | 4.10 | 1659 |
| 401558 | | | ENSP00000220478*; SECRETOGRANIN III. | 4.10 | |
| 414962 | AF273304 | Hs.235376 | XPMC2 protein | 4.10 | 4936 896 |
| 431585 | BE242803 | Hs.262823 | hypothetical protein FLJ10326 | 4.10 | 2572 |
| 455505 | AW970640 | Hs.309071 | ESTs | 4.10 | 4511 |
| 449230 | BE613348 | | melanoma cell adhesion molecule | 4.09 | 4049 |
| 434474 | AL042936 | Hs.211571 | holocytochrome c synthase (cytochrome c | 4.08 | 2842 |
| 453902 | BE502341 | Hs.3402 | ESTs | 4.08 | 4449 |
| 430387 | AW372884 | Hs.240770 | nuclear cap binding protein subunit 2, 2 | 4.08 | 2479 |
| 431183 | NM_006855 | Hs.250696 | KDEL (Lys-Asp-Glu-Leu) endoplasmic retic | 4.07 | 2537 5472 |
| 424130 | AL050136 | Hs.140945 | Homo sapiens mRNA; cDNA DKFZp586L141 (fr | 4.07 | 1806 |
| 433029 | NM_014322 | Hs.279926 | opsin 3 (encephalopsin) | 4.06 | 2726 5524 |
| 443683 | BE241717 | Hs.9676 | uncharacterized hypothalamus protein HT0 | 4.06 | 3539 |
| 415701 | NM_003878 | Hs.78619 | gamma-glutamyl hydrolase (conjugase, fol | 4.06 | 4952 945 |
| 412205 | N33818 | Hs.20274 | ESTs, Weakly similar to unnamed protein | 4.05 | 618 |
| 450193 | AI916071 | Hs.15607 | Homo sapiens Fanconi anemia complementat | 4.05 | 4111 |
| 422072 | AB018255 | Hs.111138 | KIAA0712 gene product | 4.05 | 1597 5133 |
| 433077 | AA314262 | Hs.289008 | YDD19 protein | 4.05 | 2732 |
| 413333 | M74028 | Hs.75297 | fibroblast growth factor 1 (acidic) | 4.05 | 712 |
| 417720 | AA205625 | Hs.208067 | ESTs | 4.04 | 1116 |
| 456508 | M502764 | Hs.123469 | ESTs, Weakly similar to AF208855 1 BM-01 | 4.03 | 4547 |
| 437967 | BE277414 | Hs.5947 | mel transforming oncogene (derived from | 4.03 | 3112 |
| 428471 | X57348 | Hs.184510 | stratifin | 4.02 | 2262 5361 |
| 409518 | BE384836 | Hs.3454 | KIAA1821 protein | 4.02 | 380 |
| 451468 | AW503398 | Hs.293663 | ESTs, Moderately similar I38022 hypot | 4.00 | 4224 |
| 421977 | W94197 | Hs.110165 | ribosomal protein L26 homolog | 4.00 | 1588 |
| 413413 | D82520 | | zinc finger protein 36 (KOX 18) | 4.00 | 717 |
| 420851 | AA281062 | Hs.29493 | hypothetical protein FLJ20142 | 4.00 | 1460 |
| 446873 | AI554439 | | ESTs | 4.00 | 3797 |
| 402496 | | | Target Exon | 3.98 | |
| 408784 | AW971350 | Hs.63386 | ESTs | 3.97 | 307 |
| 418004 | U37519 | Hs.87539 | aldehyde dehydrogenase 3 family, member | 3.96 | 1145 4997 |
| 447514 | AI809314 | Hs.208501 | ESTs, Weakly similar to B34087 hypotheti | 3.96 | 3872 |
| 430178 | AW449612 | Hs.152475 | 3'UTR of: achaete-scute complex (Drosoph | 3.96 | 2451 |
| 449810 | AB008681 | Hs.23994 | activin A receptor, type IIB | 3.96 | 4085 5723 |
| 422309 | U79745 | Hs.114924 | solute carrier family 16 (monocarboxylic | 3.96 | 1630 5146 |
| 437763 | AA469369 | Hs.5831 | tissue inhibitor of metalloproteinase 1 | 3.96 | 3089 |
| 430791 | AA486293 | Hs.272068 | ESTs, Weakly similar to ALU3_HUMAN ALU S | 3.96 | 2513 |
| 408761 | AA057264 | Hs.238936 | ESTs, Weakly similar to (defline not ava | 3.95 | 304 |
| 419713 | AW968058 | Hs.92381 | nudix (nucleoside diphosphate linked moi | 3.95 | 1352 |
| 426227 | U67058 | Hs.154299 | Human proteinase activated receptor-2 mR | 3.95 | 2041 |
| 448595 | AB014544 | Hs.21572 | KIAA0644 gene product | 3.95 | 3987 5711 |
| 423175 | W27595 | Hs.347310 | hypothetical protein FLJ14627 | 3.95 | 1724 |
| 425649 | U30930 | Hs.158540 | UDP glycosyltransferase 8 (UDP-galactose | 3.95 | 1982 5273 |
| 411678 | AI907114 | Hs.71465 | squalene epoxidase | 3.94 | 568 |
| 435854 | AJ278120 | Hs.4996 | putative ankyrin-repeat containing prote | 3.93 | 2949 5571 |
| 424954 | NM_000546 | Hs.1846 | tumor protein p53 (Li-Fraumeni syndrome) | 3.92 | 1901 5238 |
| 442767 | AI017208 | Hs.131149 | ESTs | 3.92 | 3467 |
| 407634 | AW016569 | Hs.136414 | UDP-GIcNAc: betaGal beta-1,3-N-acetylgluc | 3.91 | 174 |
| 457819 | AA057484 | Hs.35406 | FLJ20522 Hypothetical protein FLJ20522 | 3.91 | 4601 |
| 410418 | D31382 | Hs.63325 | transmembrane protease, serine 4 | 3.90 | 476 |
| 404704 | | | NM_001539*: Homo sapiens heat shock prote | 3.90 | 4727 60 |
| 403127 | | | polymerase (DNA directed), delta 1, cata | 3.90 | |
| 436643 | AA757626 | Hs.10941 | ESTs, Weakly similar to IPP1_HUMAN PROTE | 3.90 | 2999 |
| 416498 | U33632 | Hs.79351 | potassium channel, subfamily K, member 1 | 3.90 | 4962 999 |
| 413281 | AA861271 | Hs.222024 | transcription factor BMAL2 | 3.90 | 706 |
| 446999 | AA151520 | | hypothetical protein MGC4485 | 3.90 | 3811 |
| 429610 | AB024937 | Hs.211092 | LUNX protein; PLUNC (palate lung and nas | 3.89 | 2395 5415 |
| 446356 | AI816736 | Hs.14896 | DHHC1 protein | 3.88 | 3747 |
| 458997 | AW937420 | | ESTs | 3.88 | 4650 |
| 440074 | AA863045 | Hs.10669 | ESTs, Weakly similar to T00050 hypotheti | 3.88 | 3259 |
| 436087 | BE300296 | Hs.5054 | CGI-133 protein | 3.87 | 2963 |
| 448690 | AK001304 | Hs.21771 | Wolf-Hirschhorn syndrome candidate 2 | 3.87 | 3998 |
| 446269 | AW263155 | Hs.14559 | hypothetical protein FLJ10540 | 3.86 | 3741 |
| 449696 | AW779666 | Hs.266854 | ESTs | 3.86 | 4074 |
| 444864 | AW965446 | Hs.81907 | ESTs, Weakly similar to T26501 hypotheti | 3.85 | 3635 |
| 424921 | AA348491 | Hs.322456 | hypothetical protein DKFZp761D0211 | 3.85 | 1899 |
| 412123 | BE251328 | Hs.73291 | hypothetical protein FLJ10881 | 3.85 | 610 |
| 434725 | AK000796 | Hs.4104 | hypothetical protein | 3.85 | 2863 5553 |
| 408670 | AF160967 | Hs.46784 | potassium large conductance calcium-acti | 3.85 | 291 4803 |
| 409151 | AA306105 | Hs.50785 | SEC22, vesicle trafficking protein (S. c | 3.85 | 341 |
| 422956 | BE545072 | Hs.122579 | ECT2 protein (Epithelial cell transformi | 3.85 | 1698 |
| 421662 | NM_014141 | Hs.106552 | cell recognition molecule Caspr2 | 3.84 | 1552 5119 |
| 427674 | NM_003528 | Hs.2178 | H2B histone family, member Q | 3.84 | 2177 5342 |
| 441680 | AW444598 | Hs.7940 | RAP1, GTP-GDP dissociation stimulator 1 | 3.83 | 3368 |
| 440325 | NM_003812 | Hs.7164 | a disintegrin and metalloproteinase doma | 3.83 | 3281 5606 |
| 434423 | NM_006769 | Hs.3844 | LIM domain only 4 | 3.83 | 2839 5548 |
| 416586 | D44643 | Hs.14144 | secreted modular calcium-binding protein | 3.82 | 1005 |
| 413551 | BE242639 | Hs.75425 | ubiquitin associated protein | 3.81 | 733 |
| 425007 | AA456483 | Hs.172081 | phosphodiesterase 4D, cAMP-specific (dun | 3.81 | 1910 |
| 430399 | AI916284 | Hs.199671 | ESTs | 3.81 | 2482 |
| 429345 | R11141 | Hs.199695 | hypothetical protein | 3.81 | 2357 |
| 426722 | U53823 | Hs.171952 | occludin | 3.81 | 2091 5316 |
| 428227 | AA321649 | Hs.2248 | small inducible cytokine subfamily B (Cy | 3.80 | 2231 |
| 404632 | | | NM_022490: Homo sapiens hypothetical prot | 3.80 | 4726 59 |
| 432169 | Y00971 | Hs.2910 | phosphoribosyl pyrophosphate synthetase | 3.80 | 2637 5501 |
| 420805 | L10333 | Hs.99947 | reticulon 1 | 3.80 | 1456 5088 |
| 411704 | AI499220 | Hs.71573 | hypothetical protein FLJ10074 | 3.80 | 571 |
| 453891 | AB037751 | Hs.36353 | Homo sapiens mRNA full length insert cDN | 3.80 | 4446 5787 |
| 435513 | AW404075 | Hs.42785 | DC11 protein | 3.80 | 2922 |
| 445165 | AV652831 | Hs.234058 | gb: AV652831 GLC Homo sapiens cDNA clone | 3.80 | 3658 |
| 404571 | | | NM_015902*: Homo sapiens progestin induce | 3.80 | 4724 57 |
| 455019 | AW850818 | | gb: IL3-CT0220-091199-026-A03 CT0220 Homo | 3.79 | 4500 |
| 426501 | AW043782 | Hs.293616 | ESTs | 3.79 | 2072 |
| 407777 | AA161071 | Hs.71465 | squalene epoxidase | 3.79 | 194 |
| 442064 | AI422867 | Hs.88594 | ESTs | 3.79 | 3392 |
| 440590 | AI863446 | Hs.266308 | mosaic serine protease | 3.78 | 3301 |
| 441128 | AA570256 | | ESTs, Weakly similar to T23273 hypotheti | 3.75 | 3334 |
| 407300 | AA102616 | Hs.120769 | gb: zn43e07.s1 Stratagene HeLa cell s3 93 | 3.75 | 149 |
| 419682 | H13139 | Hs.92282 | paired-like homeodomain transcription fa | 3.75 | 1349 |
| 411598 | BE336654 | Hs.70937 | H3 histone family, member A | 3.75 | 562 |
| 453900 | AW003582 | Hs.226414 | ESTs, Weakly similar to ALU8_HUMAN ALU S | 3.75 | 4448 |
| 421202 | AF193339 | Hs.102506 | eukaryotic translation initiation factor | 3.75 | 1489 5097 |
| 423645 | AI215632 | Hs.147487 | ESTs | 3.75 | 1764 |
| 455459 | AW947654 | | gb: RCO-MT0003-140300-031-b06 MT0003 Homo | 3.75 | 4509 |
| 411450 | H49619 | Hs.127301 | ESTs | 3.75 | 557 |
| 425842 | AI587490 | Hs.159623 | NK-2 (Drosophila) homolog B | 3.75 | 2002 |
| 410935 | BE067395 | Hs.66881 | dynein, cytoplasmic, intermediate polype | 3.74 | 521 |
| 447735 | AA775268 | Hs.6127 | Homo sapiens CDNA: FLJ23020 fis, clone L | 3.74 | 3898 |
| 454417 | AI244459 | Hs.110826 | trinucleotide repeat containing 9 | 3.74 | 4480 |
| 408349 | BE546947 | Hs.44276 | homeo box C10 | 3.73 | 258 |
| 439778 | AL109729 | Hs.99364 | putative transmembrane protein | 3.73 | 3235 |
| 452012 | AA307703 | Hs.279766 | kinesin family member 4A | 3.70 | 4262 |
| 400102 | | | Eos Control | 3.70 | |
| 415621 | AI648602 | Hs.55468 | ESTs | 3.70 | 938 |
| 437866 | AA156781 | | metallothionein 1E (functional) | 3.70 | 3101 |
| 443687 | F13040 | | KIAA1228 protein | 3.70 | 3540 |
| 428690 | AI948490 | Hs.98765 | ESTs | 3.70 | 2281 |
| 450447 | AF212223 | Hs.25010 | hypothetical protein P15-2 | 3.70 | 4139 5730 |
| 444946 | AW139205 | Hs.156457 | hypothetical protein FLJ22408 | 3.70 | 3642 |
| 427779 | AA906997 | Hs.180780 | TERA protein | 3.69 | 2195 |
| 437929 | T09353 | Hs.106642 | ESTs, Weakly similar to T09052 hypotheti | 3.68 | 3108 |
| 439588 | AA838166 | Hs.174644 | hypothetical protein FLJ21669 | 3.68 | 3221 |
| 430044 | AA464510 | Hs.152812 | ESTs | 3.68 | 2439 |
| 427982 | NM_016156 | Hs.181326 | KIAA1073 protein | 3.67 | 2210 5349 |
| 421180 | BE410992 | Hs.258730 | heme-regulated initiation factor 2-alpha | 3.65 | 1486 |
| 434063 | AA018893 | Hs.3727 | unr.interacting protein | 3.65 | 2811 |
| 433701 | AW445023 | Hs.15155 | ESTs | 3.65 | 2782 |
| 422390 | AW450893 | Hs.121830 | ESTs, Weakly similar to T42682 hypotheti | 3.65 | 1638 |
| 451407 | AA131376 | Hs.343809 | fibroblast growth factor 12B | 3.65 | 4220 |
| 421991 | NM_014918 | Hs.110488 | KIAA0990 protein | 3.65 | 1589 5132 |
| 429656 | X05608 | Hs.211584 | neurofilament, tight polypeptide (68 kD) | 3.65 | 2403 5421 |
| 415068 | Z19448 | Hs.131887 | ESTs, Weakly similar to T24396 hypotheti | 3.65 | 904 |
| 416198 | H27332 | Hs.99598 | hypothetical protein MGC5338 | 3.65 | 980 |
| 425782 | U66468 | Hs.159525 | cell growth regulatory with EF-hand doma | 3.65 | 1996 5278 |
| 452512 | AW363486 | Hs.337635 | ESTs | 3.65 | 4319 |
| 446162 | AI631319 | Hs.63841 | hypothetical protein DKFZp434E2318 | 3.65 | 3730 |
| 402114 | | | Target Exon | 3.65 | |
| 437204 | AL110216 | | ESTs, Weakly similar to I55214 salivary | 3.65 | 3043 |
| 419354 | M62839 | Hs.1252 | apolipoprotein H (beta-2-glycoprotein I) | 3.65 | 1303 5044 |
| 448105 | AW591433 | Hs.298241 | Transmembrane protease, serine 3 | 3.64 | 3943 |
| 405204 | | | NM_002086*: Homo sapiens growth factor re | 3.64 | 4731 65 |
| 412314 | AA825247 | | G protein-coupled receptor 27 (GPR27) (S | 3.64 | 623 |
| 427398 | AW390020 | Hs.20415 | chromosome 21 open reading frame 11 | 3.64 | 2151 |
| 418941 | AA452970 | Hs.239527 | E1B-55kDa-associated protein 5 | 3.64 | 1260 |
| 433730 | AK002135 | Hs.3542 | hypothetical protein FLJ11273 | 3.64 | 2783 5540 |
| 456439 | AA251242 | Hs.103238 | ESTs | 3.63 | 4542 |
| 446783 | AW138343 | Hs.141867 | ESTs | 3.63 | 3786 |
| 439211 | AI890347 | Hs.271923 | Homo sapiens cDNA: FLJ22785 fis, clone K | 3.63 | 3193 |
| 450516 | AA902656 | Hs.21943 | NIF3 (Ngg1 interacting factor 3, S. pombe | 3.62 | 4141 |
| 431933 | AI187057 | Hs.132554 | ESTs | 3.62 | 2618 |
| 429084 | AJ001443 | Hs.195614 | splicing factor 3b, subunit 3, 130 kD | 3.62 | 2332 5390 |
| 432284 | AA532807 | Hs.105822 | ESTs | 3.61 | 2653 |
| 452833 | BE559681 | Hs.30736 | KIAA0124 protein | 3.61 | 4351 |
| 447702 | AI420256 | Hs.161271 | ESTs | 3.60 | 3894 |
| 449571 | AW016812 | Hs.200266 | ESTs | 3.60 | 4069 |
| 414747 | U30872 | Hs.77204 | centromere protein F (350/400 kD, mitosin | 3.60 | 4927 873 |
| 431542 | H63010 | Hs.5740 | ESTs | 3.60 | 2562 |
| 426788 | U66615 | Hs.172280 | SWI/SNF related, matrix associated, acti | 3.60 | 2099 5320 |
| 415791 | H09366 | Hs.78853 | uracil-DNA glycosylase | 3.60 | 953 |
| 440492 | R39127 | Hs.26966 | hypothetical protein DKFZp547J036 | 3.59 | 3292 |
| 457670 | AF119666 | Hs.23449 | insulin receptor tyrosine kinase substra | 3.58 | 4599 5814 |
| 409262 | AK000631 | Hs.52256 | hypothetical protein FLJ20624 | 3.57 | 355 4819 |
| 446872 | X97058 | Hs.16362 | pyrimidinergic receptor P2Y, G-protein c | 3.57 | 3796 5678 |
| 414696 | AF002020 | Hs.76918 | Niemann-Pick disease, type C1 | 3.57 | 4924 864 |
| 436134 | AK000618 | Hs.123784 | ESTs | 3.57 | 2964 |
| 427853 | AI569798 | Hs.98260 | ESTs | 3.57 | 2201 |
| 414222 | AL135173 | Hs.878 | sorbitol dehydrogenase | 3.56 | 805 |
| 411285 | AI733766 | Hs.69429 | Homo sapiens IMAGE: 512024 clone, mRNA | 3.55 | 545 |
| 453160 | AI263307 | | H2B histone family, member L | 3.55 | 4380 |
| 422094 | AF129535 | Hs.272027 | F-box only protein 5 | 3.55 | 1602 5137 |
| 438118 | AW753311 | | ESTs | 3.55 | 3119 |
| 429048 | AI372949 | Hs.44241 | Homo sapiens cDNA: FLJ21447 fis, clone C | 3.55 | 2329 |
| 432329 | NM_002962 | Hs.2960 | S100 calcium-binding protein A5 | 3.55 | 2655 5506 |
| 449318 | AW236021 | Hs.78531 | Homo sapiens, Similar to RIKEN cDNA 5730 | 3.55 | 4055 |
| 444151 | AW972917 | Hs.128749 | alpha-methylacyl-CoA racemase | 3.55 | 3577 |
| 443450 | N66045 | Hs.133529 | ESTs | 3.55 | 3524 |
| 453507 | AF083217 | Hs.33085 | WD repeat domain 3 | 3.54 | 4414 5778 |
| 432540 | AI821517 | Hs.105866 | ESTs | 3.54 | 2678 |
| 448664 | AI879317 | Hs.334691 | splicing factor 3a, subunit 1, 120 kD | 3.54 | 3994 |
| 402855 | | | NM_001839*: Homo sapiens calponin 3, acid | 3.54 | 40 4709 |
| 453751 | R36762 | Hs.101282 | Homo sapiens cDNA: FLJ21238 fis, clone C | 3.53 | 4428 |
| 403817 | | | NM_015271: Homo sapiens tripartite motif- | 3.53 | 4716 48 |
| 427471 | AA403131 | Hs.266782 | KIAA1826 protein | 3.52 | 2154 |
| 431387 | AI878854 | Hs.252229 | v-maf musculoaponeurotic fibrosarcoma (a | 3.52 | 2552 |
| 415947 | U04045 | Hs.78934 | mutS (E. coli) homolog 2 (colon cancer, | 3.52 | 4955 962 |
| 438523 | H66220 | Hs.278177 | ESTs | 3.51 | 3144 |
| 452874 | AK001061 | Hs.30925 | hypothetical protein FLJ10199 | 3.51 | 4355 5766 |
| 453033 | AA325869 | Hs.31463 | KIAA0281 gene product | 3.50 | 4369 |
| 439605 | AF086431 | Hs.22380 | ESTs | 3.50 | 3223 |
| 453785 | AI368236 | Hs.283732 | ESTs, Moderately similar to ALU1_HUMAN A | 3.50 | 4432 |
| 434970 | AW272262 | Hs.225767 | ESTs | 3.50 | 2884 |
| 406081 | | | Target Exon | 3.50 | |
| 430712 | AW044647 | | ESTs | 3.50 | 2507 |
| 420912 | AW853156 | Hs.90787 | ESTs | 3.50 | 1463 |
| 411096 | U80034 | Hs.68583 | mitochondrial intermediate peptidase | 3.50 | 4862 535 |
| 418338 | NM_002522 | Hs.84154 | neuronal pentraxin I | 3.50 | 1189 5010 |
| 409902 | AI337658 | Hs.156351 | ESTs | 3.50 | 416 |
| 406685 | M18728 | | gb: Human nonspecific crossreacting antig | 3.49 | 4745 83 |
| 442045 | C05768 | Hs.8078 | Homo sapiens clone FBD3 Cri-du-chat crit | 3.49 | 3390 |
| 440773 | AA352702 | Hs.37747 | Homo sapiens, Similar to RIKEN cDNA 2700 | 3.49 | 3318 |
| 408393 | AW015318 | Hs.23165 | ESTs | 3.49 | 263 |
| 446825 | BE266822 | Hs.344097 | filamin A, alpha (actin-binding protein- | 3.48 | 3792 |
| 450937 | R49131 | Hs.26267 | ATP-dependant interferon response protei | 3.48 | 4177 |
| 421948 | L42583 | Hs.334309 | keratin 6A | 3.47 | 1583 5130 |
| 448296 | BE622756 | Hs.10949 | Homo sapiens cDNA FLJ14162 fis, clone NT | 3.47 | 3956 |
| 459709 | AA653774 | Hs.212084 | ESTs | 3.47 | 4679 |
| 412507 | L36645 | Hs.73964 | EphA4 | 3.46 | 4880 639 |
| 442426 | AI373062 | Hs.332938 | hypothetical protein MGC5370 | 3.46 | 3427 |
| 438956 | W00847 | Hs.135056 | Human DNA sequence from clone RP5-850E9 | 3.46 | 3180 |
| 435092 | AL137310 | Hs.4749 | Homo sapiens mRNA; cDNA DKFZp761E13121 ( | 3.45 | 2895 5562 |
| 443347 | AI052543 | Hs.133244 | melanoma-derived leucine zipper, extra-n | 3.45 | 3519 |
| 411261 | AW834655 | | gb: MR2-TT0014-291199-017-g11 TT0014 Homo | 3.45 | 543 |
| 416658 | U03272 | Hs.79432 | fibrillin 2 (congenital contractural ara | 3.45 | 1011 4966 |
| 453941 | U39817 | Hs.36820 | Bloom syndrome | 3.45 | 4454 5790 |
| 442353 | BE379594 | Hs.49136 | ESTs, Moderately similar to ALU7_HUMAN A | 3.45 | 3423 |
| 410206 | NM_003826 | Hs.60415 | N-ethylmaleimide-sensitive factor attach | 3.45 | 454 4840 |
| 441865 | AA384726 | Hs.5722 | hypothetical protein FLJ23316 | 3.45 | 3376 |
| 414602 | AW630088 | Hs.76550 | Homo sapiens mRNA; cDNA DKFZp564B1264 (f | 3.44 | 853 |
| 432229 | AW290976 | Hs.143587 | ESTs | 3.43 | 2646 |
| 434808 | AF155108 | Hs.256150 | NY-REN-41 antigen | 3.43 | 2868 5554 |
| 415632 | U67085 | Hs.78524 | TcD37 homolog | 3.43 | 4950 939 |
| 441790 | AW294909 | Hs.132208 | ESTs | 3.42 | 3372 |
| 438697 | AA814439 | | ESTs | 3.42 | 3157 |
| 452827 | AI571835 | Hs.55468 | ESTs | 3.42 | 4350 |
| 441805 | AA285136 | Hs.301914 | neuronal specific transcription factor D | 3.42 | 3373 |
| 433149 | BE257672 | Hs.42949 | hypothetical protein HES6 | 3.42 | 2740 |
| 438832 | AA827023 | Hs.270981 | hypothetical protein FLJ14397 | 3.41 | 3169 |
| 425548 | AA890023 | Hs.1906 | prolactin receptor | 3.41 | 1978 |
| 406304 | | | transcriptional adaptor 3 (ADA3, yeast h | 3.40 | |
| 434844 | AF157116 | Hs.22350 | hypothetical protein LOC56757 | 3.40 | 2873 |
| 447669 | AL049985 | Hs.19180 | Homo sapiens mRNA; cDNA DKFZp564E122 (fr | 3.40 | 3888 |
| 400884 | | | Target Exon | 3.40 | |
| 411683 | W01167 | Hs.50426 | ESTs | 3.40 | 569 |
| 445242 | BE156478 | Hs.21108 | ESTs, Weakly similar to ALU1_HUMAN ALU S | 3.40 | 3661 |
| 424796 | AW298244 | Hs.266195 | ESTs | 3.40 | 1887 |
| 422358 | AL133030 | Hs.115429 | Homo sapiens mRNA for KIAA1666 protein, | 3.40 | 1635 5149 |
| 404995 | | | ENSP00000251890*: Monocytic leukemia zinc | 3.40 | |
| 438525 | AW368528 | Hs.100855 | ESTs | 3.40 | 3145 |
| 415263 | AA948033 | Hs.130853 | ESTs | 3.40 | 920 |
| 428753 | AW939252 | Hs.192927 | hypothetical protein FLJ20251 | 3.40 | 2291 |
| 449343 | AI151418 | | protein phosphatase 3 (formely 2B), cat | 3.40 | 4056 |
| 412863 | AA121673 | Hs.59757 | zinc finger protein 281 | 3.40 | 679 |
| 424036 | AA770688 | | H2A histone family, member L | 3.40 | 1793 |
| 452823 | AB012124 | Hs.30696 | transcription factor-like 5 (basic helix | 3.40 | 4348 5764 |
| 408437 | AW957744 | Hs.278469 | lacrimal proline rich protein | 3.39 | 267 |
| 415402 | AA164687 | Hs.177576 | mannosyl (alpha-1,3-)-glycoprotein beta- | 3.39 | 930 |
| 412722 | AI343300 | Hs.15091 | ESTs | 3.38 | 664 |
| 426793 | X89887 | Hs.172350 | HIR (histone cell cycle regulation defec | 3.38 | 2100 5321 |
| 417366 | BE185289 | Hs.1076 | small proline-rich protein 1B (cornifin) | 3.38 | 1087 |
| 422611 | AA158177 | Hs.118722 | fucosyltransferase 8 (alpha (1,6) fucosy | 3.38 | 1662 |
| 452598 | AI831594 | | ESTs, Weakly similar to ALU7_HUMAN ALU S | 3.38 | 4328 |
| 406981 | S71129 | | acetylcholinesterase (YT blood group) | 3.38 | 117 4761 |
| 414844 | AA296874 | Hs.77494 | deoxyguanosine kinase | 3.37 | 884 |
| 409235 | AA188827 | Hs.7988 | ESTs, Weakly similar to I38022 hypotheti | 3.37 | 354 |
| 436608 | AA628980 | Hs.192371 | down syndrome critical region protein DS | 3.36 | 2997 |
| 415474 | NM_014252 | Hs.78457 | solute carrier family 25 (mitochondrial | 3.36 | 4948 934 |
| 447850 | AB018298 | Hs.19822 | SEC24 (S. cerevisiae) related gene famil | 3.36 | 3914 5702 |
| 400583 | | | Target Exon | 3.36 | |
| 419138 | U48508 | Hs.89631 | ryanodine receptor 1 (skeletal) | 3.36 | 1281 5039 |
| 430188 | AL049242 | Hs.234794 | Homo sapiens mRNA; cDNA DKFZp564B083 (fr | 3.35 | 2453 |
| 452323 | W44356 | Hs.292812 | ESTs, Weakly similar to T33468 hypotheti | 3.35 | 4296 |
| 437534 | AA814471 | Hs.23510 | ESTs | 3.35 | 3068 |
| 441420 | AA932872 | | gb: oo57d07.s1 NCI_CGAP_Lu5 Homo sapiens | 3.35 | 3352 |
| 420337 | AW295840 | Hs.14555 | Homo sapiens cDNA: FLJ21513 fis, clone C | 3.35 | 1414 |
| 408101 | AW968504 | Hs.123073 | CDC2-related protein kinase 7 | 3.35 | 234 |
| 425769 | U72513 | Hs.159486 | Human RPL13-2 pseudogene mRNA, complete | 3.35 | 1995 |
| 451806 | NM_003729 | Hs.27076 | RNA 3′-terminal phosphate cyclase | 3.35 | 4248 5751 |
| 409673 | AK000611 | Hs.55781 | hypothetical protein FLJ20604 | 3.35 | 393 4830 |
| 410166 | AK001376 | Hs.59346 | hypothetical protein FLJ10514 | 3.35 | 451 4839 |
| 412673 | AL042957 | Hs.31845 | ESTs | 3.35 | 659 |
| 442326 | H92962 | Hs.124813 | hypothetical protein MGC14817 | 3.35 | 3417 |
| 410660 | AI061118 | Hs.65328 | Fanconi anemia, complementation group F | 3.35 | 502 |
| 442660 | AW138174 | Hs.130651 | ESTs | 3.35 | 3458 |
| 442573 | H93366 | Hs.7567 | branched chain aminotransferase 1, cytos | 3.35 | 3446 |
| 447644 | AW861622 | Hs.108646 | Homo sapiens cDNA FLJ14934 fis, clone PL | 3.35 | 3886 |
| 448752 | AA593867 | Hs.300842 | KIAA1608 protein | 3.34 | 4003 |
| 402537 | | | C1003560*: gi\|10435919\|dbj\|BAB14704.1\| (A | 3.33 | |
| 432834 | F06459 | Hs.289113 | cytochrome b5 reductase 1 (B5R.1) | 3.33 | 2703 |
| 418721 | NM_002731 | Hs.87773 | protein kinase, cAMP-dependent, catalyti | 3.33 | 1231 5027 |
| 425424 | NM_004954 | Hs.157199 | ELKL motif kinase | 3.33 | 1962 5263 |
| 434340 | AI193043 | Hs.128685 | ESTs, Weakly similar to T17226 hypotheti | 3.32 | 2830 |
| 443209 | AI040125 | Hs.150521 | ESTs | 3.32 | 3508 |
| 401560 | | | heparan sulfate (glucosamine) 3-O-sulfot | 3.31 | |
| 447832 | AI433357 | | ESTs | 3.31 | 3911 |
| 453469 | AB014533 | Hs.33010 | KIAA0633 protein | 3.31 | 4412 5777 |
| 426600 | NM_003378 | Hs.171014 | VGF nerve growth factor inducible | 3.30 | 2081 5312 |
| 435770 | AA699991 | Hs.348162 | gb: zi69a09.s1 Soares_fetal_liver_spleen_{—} | 3.30 | 2942 |
| 410491 | AA465131 | Hs.64001 | Homo sapiens clone 25218 mRNA sequence | 3.30 | 484 |
| 444070 | NM_015367 | Hs.10267 | MIL1 protein | 3.30 | 3571 5635 |
| 424332 | AA338919 | Hs.101615 | ESTs | 3.30 | 1830 |
| 421768 | AI923934 | Hs.108112 | histone fold protein CHRAC17; DNA polyme | 3.30 | 1560 |
| 425018 | BE245277 | Hs.154196 | E4F transcription factor 1 | 3.30 | 1912 |
| 430187 | AI799909 | Hs.158989 | ESTs | 3.30 | 2452 |
| 452234 | AW084176 | Hs.223296 | ESTs, Weakly similar to I38022 hypotheti | 3.30 | 4284 |
| 440138 | AB033023 | Hs.318127 | hypothetical protein FLJ10201 | 3.30 | 3267 5603 |
| 444743 | AA045648 | Hs.301957 | nudix (nucleoside diphosphate linked moi | 3.30 | 3624 |
| 411019 | AW993097 | Hs.48617 | Homo sapiens cDNA FLJ12540 fis, clone NT | 3.30 | 528 |
| 438378 | AW970529 | Hs.86434 | hypothetical protein FLJ21816 | 3.30 | 3136 |
| 407204 | R41933 | Hs.140237 | ESTs, Weakly similar to ALU1_HUMAN ALU S | 3.29 | 136 |
| 440704 | M69241 | Hs.162 | insulin-like growth factor binding prote | 3.29 | 3314 5613 |
| 441622 | AW450957 | Hs.224864 | ESTs | 3.29 | 3361 |
| 425840 | AW978731 | Hs.301824 | hypothetical protein PRO1331 | 3.29 | 2001 |
| 440006 | AK000517 | Hs.6844 | NALP2 protein; PYRIN-Containing APAF1-li | 3.29 | 3252 5601 |
| 450291 | AW015549 | Hs.269199 | ESTs | 3.28 | 4124 |
| 440282 | BE262386 | | clones 23667 and 23775 zinc finger prote | 3.27 | 3277 |
| 440675 | AW005054 | Hs.279788 | ESTs, Weakly similar to KCC1_HUMAN CALCI | 3.27 | 3312 |
| 424259 | AK001776 | Hs.143954 | hypothetical protein FLJ10914 | 3.26 | 1822 5207 |
| 431130 | NM_006103 | Hs.2719 | HE4; epididymis-specific, whey-acidic pr | 3.26 | 2533 5470 |
| 436557 | W15573 | Hs.5027 | ESTs, Weakly similar to A47582 B-cell gr | 3.26 | 2993 |
| 448045 | AJ297436 | Hs.20166 | prostate stem cell antigen | 3.26 | 3937 5706 |
| 409406 | H83092 | Hs.49605 | ESTs | 3.25 | 366 |
| 416677 | T83470 | Hs.334840 | ESTs, Moderately similar to I78885 serin | 3.25 | 1012 |
| 422771 | NM_012318 | Hs.120165 | leucine zipper-EF-hand containing transm | 3.25 | 1681 5158 |
| 435005 | U80743 | Hs.306094 | trinucleotide repeat containing 12 | 3.25 | 2886 5560 |
| 422134 | AW179019 | Hs.112110 | mitochondrial ribosomal protein L42 | 3.25 | 1608 |
| 442152 | R39246 | Hs.239666 | Homo sapiens cDNA FLJ13495 fis, clone PL | 3.25 | 3401 |
| 434804 | AA649530 | | gb: ns44f05.s1 NCI_CGAP_Alv1 Homo sapiens | 3.25 | 2866 |
| 422744 | AW268803 | Hs.119640 | hBKLF for basic kruppel like factor | 3.25 | 1678 |
| 422420 | U03398 | Hs.1524 | tumor necrosis factor (ligand) superfami | 3.25 | 1643 5150 |
| 435532 | AW291488 | Hs.117305 | Homo sapiens, clone IMAGE: 3682908, mRNA | 3.25 | 2923 |
| 429413 | NM_014058 | Hs.201877 | DESC1 protein | 3.25 | 2366 5405 |
| 404148 | | | NM_002944*: Homo sapiens v-ros avian UR2 | 3.25 | 4720 53 |
| 433183 | AF231338 | Hs.222024 | transcription factor BMAL2 | 3.25 | 2745 5531 |
| 414523 | AU076633 | Hs.76353 | serine (or cysteine) proteinase inhibito | 3.25 | 841 |
| 451418 | BE387790 | Hs.26369 | hypothetical protein FLJ20287 | 3.25 | 4221 |
| 427521 | AW973352 | | ESTs | 3.25 | 2159 |
| 411945 | AL033527 | Hs.92137 | L-myc-2 protein (MYCL2) | 3.25 | 4870 590 |
| 447155 | AA100605 | Hs.121557 | ESTs, Weakly similar to DP1_HUMAN POLYPO | 3.24 | 3827 |
| 452737 | AK001680 | Hs.30488 | DKFZP434F091 protein | 3.24 | 4340 5762 |
| 411263 | BE297802 | Hs.69360 | kinesin-like 6 (mitotic centromere-assoc | 3.24 | 544 |
| 435369 | BE385785 | Hs.323715 | methionine adenosyltransferase I, alpha | 3.24 | 2910 |
| 410741 | Z11695 | Hs.324473 | mitogen-activated protein kinase 1 | 3.23 | 4856 513 |
| 415192 | D17793 | Hs.78183 | aldo-keto reductase family 1, member C3 | 3.23 | 4942 914 |
| 455416 | AW937143 | | gb: PM1-DT0041-281299-001-f01 DT0041 Homo | 3.23 | 4507 |
| 458187 | D56919 | Hs.265848 | myomegalin | 3.23 | 4614 |
| 413129 | AF292100 | Hs.104613 | RP42 homolog | 3.23 | 4893 693 |
| 415992 | C05837 | Hs.145807 | hypothetical protein FLJ13593 | 3.23 | 964 |
| 416883 | AW140128 | Hs.184902 | ESTs | 3.23 | 1033 |
| 414013 | AA766605 | Hs.47099 | hypothetical protein FLJ21212 | 3.23 | 774 |
| 453454 | AW052006 | | PRP4/STK/WD splicing factor | 3.23 | 4407 |
| 426181 | AA371422 | Hs.334371 | hypothetical protein MGC13096 | 3.22 | 2037 |
| 436995 | AI160015 | Hs.125489 | ESTs | 3.22 | 3026 |
| 428878 | AA436884 | Hs.48926 | ESTs | 3.22 | 2315 |
| 411248 | AA551538 | Hs.69321 | Homo sapiens cDNA FLJ14408 fis, clone HE | 3.21 | 541 |
| 419381 | AB023420 | Hs.90093 | heat shock 70 kD protein 4 | 3.21 | 1308 5046 |
| 427490 | Z95152 | Hs.178695 | mitogen-activated protein kinase 13 | 3.21 | 2157 |
| 417601 | NM_014735 | Hs.82292 | KIAA0215 gene product | 3.21 | 1105 4991 |
| 440273 | AI805392 | Hs.325335 | Homo sapiens cDNA: FLJ23523 fis, clone L | 3.21 | 3274 |
| 404891 | | | Target Exon | 3.20 | |
| 438989 | AW979100 | | gb: EST391210 MAGE resequences, MAGP Homo | 3.20 | 3181 |
| 403416 | | | KIAA0564 protein | 3.20 | |
| 454731 | BE067650 | | gb: MR4-BT0358-090300-003-e01 BT0358 Homo | 3.20 | 4494 |
| 414888 | AL039185 | Hs.77558 | thyroid hormone receptor interactor 7 | 3.20 | 888 |
| 420650 | AA455706 | Hs.44581 | heat shock protein hsp70-related protein | 3.20 | 1441 |
| 448686 | AA158659 | Hs.334712 | hypothetical protein FLJ14744 | 3.20 | 3997 |
| 450728 | AW162923 | Hs.25363 | presenilin 2 (Alzheimer disease 4) | 3.20 | 4156 |
| 426581 | AB040956 | Hs.135890 | KIAA1523 protein | 3.20 | 2080 5311 |
| 442179 | AA983842 | Hs.333555 | chromosome 2 open reading frame 2 | 3.20 | 3404 |
| 422536 | AA311915 | Hs.187726 | gb: EST182621 Jurkat T-cells VI Homo sapi | 3.20 | 1654 |
| 447492 | AI381619 | Hs.20188 | ESTs | 3.20 | 3867 |
| 457292 | AI921270 | Hs.281462 | hypothetical protein FLJ14251 | 3.20 | 4586 |
| 449704 | AK000733 | Hs.23900 | GTPase activating protein | 3.20 | 4076 5722 |
| 442577 | AA292998 | Hs.163900 | ESTs | 3.20 | 3447 |
| 423942 | AF209704 | Hs.169407 | glycolipid transfer protein | 3.20 | 1785 5190 |
| 439696 | W95298 | Hs.171882 | ESTs | 3.19 | 3230 |
| 409142 | AL136877 | Hs.50758 | SMC4 (structural maintenance of chromoso | 3.19 | 340 4814 |
| 446639 | AI016826 | Hs.342148 | ESTs | 3.19 | 3774 |
| 452144 | AA032197 | Hs.102558 | Homo sapiens, clone MGC: 5352, mRNA, comp | 3.18 | 4275 |
| 446054 | AB014537 | Hs.13604 | KIAA0637 gene product | 3.18 | 3722 5664 |
| 452126 | AI859947 | Hs.314158 | ESTs, Weakly similar to I38022 hypotheti | 3.18 | 4274 |
| 426108 | AA622037 | Hs.166468 | programmed cell death 5 | 3.18 | 2028 |
| 451621 | AI879148 | Hs.26770 | fatty acid binding protein 7, brain | 3.17 | 4235 |
| 433913 | AI694106 | Hs.72325 | ESTs, Weakly similar to I38022 hypotheti | 3.17 | 2797 |
| 427254 | AL121523 | Hs.97774 | ESTs | 3.16 | 2135 |
| 430361 | AI033965 | Hs.239926 | sterol-C4-methyl oxidase-like | 3.16 | 2476 |
| 450065 | AL050107 | Hs.24341 | transcriptional co-activator with PDZ-bi | 3.16 | 4102 5725 |
| 420090 | AA220238 | Hs.94986 | ribonuclease P (38 kD) | 3.16 | 1383 |
| 436481 | AA379597 | Hs.5199 | HSPC150 protein similar to ubiquitin-con | 3.16 | 2988 |
| 435652 | N32388 | Hs.334370 | uncharacterized hypothalamus protein HBE | 3.15 | 2935 |
| 453905 | NM_002314 | Hs.36566 | LIM domain kinase 1 | 3.15 | 4450 5788 |
| 454425 | AW300927 | Hs.27192 | hypothetical protein dJ1057B20.2 | 3.15 | 4482 |
| 425419 | AJ002078 | Hs.157144 | syntaxin 6 | 3.15 | 1961 5262 |
| 414271 | AK000275 | Hs.75871 | protein kinase C binding protein 1 | 3.15 | 807 |
| 455751 | BE075281 | | gb: PM1-BT0585-290200-005-d07 BT0585 Homo | 3.15 | 4521 |
| 408673 | BE208517 | | ribosomal protein L37a | 3.15 | 292 |
| 405031 | | | solute carrier family 22 (organic cation | 3.15 | |
| 401216 | | | C12000969: gi\|7662320\|ref\|NP_055628.1\| KI | 3.15 | |
| 409463 | AI458165 | Hs.17296 | hypothetical protein MGC2376 | 3.15 | 375 |
| 418506 | AA084248 | Hs.85339 | Unknown protein for MGC: 29643 (formerly | 3.15 | 1208 |
| 414396 | BE548266 | Hs.76057 | galactose-4-epimerase, UDP- | 3.14 | 821 |
| 418113 | AI272141 | Hs.83484 | SRY (sex determining region Y)-box4 | 3.14 | 1161 |
| 433257 | AA613437 | Hs.302743 | hypothetical protein FLJ12543 | 3.14 | 2751 |
| 442445 | AA082665 | Hs.209561 | KIAA1715 protein | 3.13 | 3431 |
| 428375 | T65153 | Hs.104613 | ESTs | 3.13 | 2246 |
| 430713 | AA351647 | Hs.2642 | eukaryotic translation elongation factor | 3.13 | 2508 |
| 421116 | T19132 | Hs.101850 | retinol-binding protein 1, cellular | 3.13 | 1482 |
| 434573 | AW372340 | Hs.14337 | ESTs | 3.13 | 2850 |
| 448102 | AI750793 | Hs.20295 | CHK1 (checkpoint, S.pombe) homolog | 3.13 | 3942 |
| 425159 | NM_004341 | Hs.154868 | carbamoyl-phosphate synthetase 2, aspart | 3.12 | 1931 5249 |
| 451336 | AI264643 | Hs.3610 | ESTs | 3.12 | 4212 |
| 423613 | AF036035 | Hs.129910 | hyaluronoglucosaminidase 3 | 3.12 | 1761 5183 |
| 430317 | AB020645 | Hs.239189 | glutaminase | 3.12 | 2471 5446 |
| 423541 | AA296922 | Hs.129778 | serine protease inhibitor, Kazal type 4 | 3.12 | 1756 |
| 403851 | | | C5002154*: gi\|7299015\|gb\|AAF54217.1\| (AE0 | 3.12 | |
| 418422 | AW440068 | Hs.59425 | hypothetical protein FLJ23323 | 3.11 | 1199 |
| 427709 | AI631811 | Hs.180403 | STRIN protein | 3.10 | 2186 |
| 440546 | AI491994 | | gb: to07g09.x1 NCl_CGAP_Ut2 Homo sapiens | 3.10 | 3298 |
| 437605 | AW452716 | Hs.13328 | hypothetical protein FLJ20421 | 3.10 | 3074 |
| 442543 | AI869950 | Hs.216039 | ESTs | 3.10 | 3438 |
| 434438 | AI654867 | | hemoglobin, beta | 3.10 | 2840 |
| 410627 | AA181339 | Hs.929 | myosin, heavy polypeptide 7, cardiac mus | 3.10 | 499 |
| 449505 | AI653006 | Hs.195374 | ESTs | 3.10 | 4063 |
| 455510 | AA422029 | Hs.143640 | ESTs, Weakly similar to hyperpolarizatio | 3.10 | 4512 |
| 446715 | AI337735 | Hs.173919 | ESTs, Moderately similar to ZN91_HUMAN Z | 3.10 | 3778 |
| 424539 | L02911 | Hs.150402 | Activin A receptor, type I (ACVR1) (ALK | 3.10 | 1857 5216 |
| 414040 | N58513 | Hs.32171 | ESTs | 3.10 | 776 |
| 443054 | AI745185 | Hs.84520 | yes-associated protein 65 kDa | 3.10 | 3491 |
| 425641 | D79758 | Hs.14355 | Homo sapiens cDNA FLJ13207 fis, clone NT | 3.10 | 1981 |
| 423642 | AW452650 | Hs.157148 | hypothetical protein MGC13204 | 3.10 | 1763 |
| 459647 | R34107 | Hs.321450 | pregnancy specific beta-1-glycoprotein 1 | 3.10 | 4675 |
| 422005 | BE266556 | Hs.110702 | Homo sapiens mRNA; cDNA DKFZp761E212 (fr | 3.10 | 1592 |
| 431868 | BE246400 | Hs.285176 | acetyl-Coenzyme A transporter | 3.10 | 2611 |
| 442530 | AI580830 | Hs.176508 | Homo sapiens cDNA FLJ14712 fis, clone NT | 3.10 | 3437 |
| 433819 | AW511097 | Hs.112765 | ESTs | 3.10 | 2789 |
| 453377 | AI679149 | Hs.167186 | ESTs | 3.10 | 4402 |
| 449000 | U69560 | Hs.3826 | kelch-like protein C3IP1 | 3.10 | 4025 |
| 411943 | BE502436 | Hs.7962 | ESTs, Weakly similar to S44608 C02F5.6 p | 3.10 | 589 |
| 421610 | AA393168 | Hs.90034 | hypothetical protein FLJ21916 | 3.10 | 1544 |
| 422947 | AA306782 | Hs.122552 | G-2 and S-phase expressed 1 | 3.10 | 1696 |
| 434669 | AF151534 | Hs.92023 | core histone macroH2A2.2 | 3.09 | 2860 5552 |
| 437108 | AA434054 | Hs.80624 | hypothetical protein MGC2560 | 3.09 | 3034 |
| 445033 | AV652402 | Hs.72901 | cyclin-dependent kinase inhibitor 2B (p1 | 3.09 | 3647 |
| 445138 | H99085 | | gb: yx18c06.s1 Soares melanocyte 2NbHM Ho | 3.09 | 3655 |
| 429901 | AK000502 | Hs.56237 | hypothetical protein FLJ20495 | 3.09 | 2424 5429 |
| 420028 | AB014680 | Hs.8786 | carbohydrate (N-acetylglucosamine-6-O) s | 3.09 | 1374 5060 |
| 436440 | AI471862 | Hs.196008 | Homo sapiens cDNA FLJ11723 fis, clone HE | 3.09 | 2985 |
| 442366 | AA115629 | Hs.118531 | ESTs | 3.09 | 3425 |
| 422684 | BE561617 | Hs.119192 | H2A histone family, member Z | 3.09 | 1673 |
| 412122 | AW852707 | | G-rich RNA sequence binding factor 1 | 3.09 | 609 |
| 440967 | AK002092 | Hs.7570 | hypothetical protein FLJ11230 | 3.08 | 3322 5615 |
| 410211 | NM_014347 | Hs.296365 | zinc finger protein | 3.08 | 456 4841 |
| 447245 | AK001713 | Hs.17860 | hypothetical protein FLJ10851 | 3.08 | 3834 5684 |
| 422385 | BE549407 | Hs.115823 | ribonuclease P, 40 kD subunit | 3.08 | 1637 |
| 437158 | AW090198 | | KIAA1150 protein | 3.07 | 3039 |
| 424395 | AA165082 | Hs.146388 | microtubute-associated protein 7 | 3.07 | 1839 |
| 418478 | U38945 | Hs.1174 | cyclin-dependent kinase inhibitor 2A (me | 3.07 | 1204 5017 |
| 428423 | AU076517 | Hs.184276 | solute carrier family 9 (sodium/hydrogen | 3.07 | 2254 |
| 429504 | X99133 | Hs.204238 | lipocalin 2 (oncogene 24p3) (NGAL) | 3.07 | 2382 5411 |
| 444700 | NM_003645 | Hs.11729 | fatty-acid-Coenzyme A ligase, very long- | 3.06 | 3620 5642 |
| 442092 | AW578669 | | hypothetical protein FLJ12439 | 3.06 | 3397 |
| 432201 | AI538613 | Hs.298241 | Transmembrane protease, serine 3 | 3.06 | 2643 |
| 446390 | AA233393 | Hs.14992 | hypothetical protein FLJ11151 | 3.05 | 3749 |
| 430396 | D49742 | Hs.241363 | hyaluronan-binding protein 2 | 3.05 | 2481 5450 |
| 446440 | AV658411 | Hs.42656 | KIAA1681 protein | 3.05 | 3751 |
| 429873 | AW268693 | Hs.105713 | ESTs | 3.05 | 2420 |
| 401203 | | | Target Exon | 3.05 | |
| 458059 | AW015588 | Hs.137232 | ESTs, Weakly similar to S65657 alpha-1C- | 3.05 | 4607 |
| 417975 | AA641836 | Hs.30085 | hypothetical protein FLJ23186 | 3.05 | 1143 |
| 459672 | Z18867 | Hs.326843 | gb: HSDHEGC03 Stratagene cDNA library Hum | 3.05 | 4676 |
| 451365 | AI791783 | Hs.16063 | ESTs, Weakly similar to ALU8_HUMAN ALU S | 3.05 | 4215 |
| 450687 | AA495800 | | gb: zw05b07.s1 Soares_NhHMPu_S1 Homo sapi | 3.05 | 4149 |
| 410976 | R36207 | Hs.25092 | hypothetical protein MGC10744 | 3.05 | 525 |
| 441318 | AI078234 | | ESTs | 3.05 | 3345 |
| 433553 | AA825523 | | ESTs, Weakly similar to I38022 hypotheti | 3.05 | 2767 |
| 447437 | U07225 | Hs.339 | purinergic receptor P2Y, G-protein coupl | 3.05 | 3861 5688 |
| 454442 | AW816134 | Hs.132967 | gb: MR3-ST0220-290100-016-e04 ST0220 Homo | 3.05 | 4484 |
| 407876 | NM_004519 | Hs.40866 | potassium voltage-gated channel, KQT-lik | 3.05 | 210 4784 |
| 410507 | AA355288 | | transitional epithelia response protein | 3.05 | 486 |
| 404440 | | | NM_021048: Homo sapiens melanoma antigen, | 3.05 | 4721 54 |
| 419865 | NM_007020 | Hs.93502 | U1-snRNP binding protein homolog (70 kD) | 3.05 | 1364 5058 |
| 410561 | BE540255 | Hs.6994 | Homo sapiens cDNA: FLJ22044 fis, clone H | 3.05 | 493 |
| 418248 | NM_005000 | Hs.83916 | NM_005000*: Homo sapiens NADH dehydrogena | 3.05 | 1172 5005 |
| 450824 | R09055 | Hs.22011 | ESTs, Weakly similar to I38022 hypotheti | 3.05 | 4169 |
| 426216 | N77630 | Hs.13895 | Homo sapiens cDNA FLJ11654 fis, clone HE | 3.05 | 2040 |
| 443098 | AI247833 | Hs.139265 | ESTs, Moderately similar to ALU1_HUMAN A | 3.04 | 3496 |
| 414388 | BE281412 | | gb: 601154911F1 NIH_MGC_21 Homo sapiens c | 3.04 | 819 |
| 427308 | D26067 | Hs.174905 | KIAA0033 protein | 3.04 | 2141 5335 |
| 451035 | AU076785 | Hs.430 | plastin 1 (I isoform) | 3.04 | 4184 |
| 440726 | AL050333 | Hs.306425 | DKFZPS64B116 protein | 3.04 | 3315 |
| 441991 | AW844404 | Hs.126901 | Homo sapiens mRNA full length insert cDN | 3.04 | 3386 |
| 433090 | AI720050 | | immortalization-upregulated protein | 3.04 | 2734 |
| 407792 | AI077715 | Hs.39384 | putative secreted ligand homologous to f | 3.04 | 196 |
| 435641 | AI147545 | Hs.114172 | ESTs | 3.03 | 2933 |
| 417542 | J04129 | Hs.82269 | progestagen-associated endometrial prote | 3.03 | 1101 4990 |
| 425997 | AK000086 | Hs.165948 | hypothetical protein FLJ20079 | 3.03 | 2018 5286 |
| 439924 | AI985897 | Hs.125293 | ESTs | 3.03 | 3242 |
| 453968 | AA847843 | Hs.62711 | High mobility group (nonhistone chromoso | 3.03 | 4456 |
| 415015 | NM_002109 | Hs.77798 | histidyl-tRNA synthetase | 3.02 | 4939 901 |
| 400183 | | | Eos Control | 3.02 | |
| 448826 | AI580252 | Hs.293246 | ESTs, Weakly similar to putative p150 [H | 3.01 | 4012 |
| 420085 | AI741909 | Hs.44680 | hypothetical protein FLJ20979 | 3.01 | 1381 |
| 429248 | U96759 | Hs.198307 | von Hippel-Lindau binding protein 1 | 3.01 | 2342 5396 |
| 409892 | AW956113 | Hs.7149 | gb: EST368183 MAGE resequences, MAGD Homo | 3.00 | 414 |
| 405942 | | | Target Exon | 3.00 | |
| 455286 | BE144384 | | gb: MR0-HT0166-191199-004-c11 HT0166 Homo | 3.00 | 4504 |
| 420301 | AA767526 | Hs.22030 | paired box gene 5 (B-cell lineage specif | 3.00 | 1407 |
| 407382 | AA503620 | | gb: ne49b08.s1 NCI_CGAP_Co3 Homo sapiens | 3.00 | 161 |
| 403011 | | | ENSP00000215330*: Probable serine/threoni | 3.00 | |
| 445076 | AI206888 | Hs.154131 | ESTs | 3.00 | 3650 |
| 418857 | D10216 | Hs.89394 | POU domain, class 1, transcription facto | 3.00 | 1250 5031 |
| 426572 | AB037783 | Hs.170623 | hypothetical protein FLJ11183 | 3.00 | 2079 5310 |
| 411506 | T75366 | Hs.146286 | kinesin family member 13A | 3.00 | 558 |
| 434301 | AF124512 | Hs.221660 | blood vessel epicardial substance | 3.00 | 2827 5545 |
| 412530 | AA766268 | Hs.266273 | hypothetical protein FLJ13346 | 3.00 | 843 |
| 403882 | | | Target Exon | 3.00 | |
| 458918 | H58499 | Hs.252692 | ESTs, Weakly similar to I38022 hypotheti | 3.00 | 4646 |
| 445704 | AI493742 | Hs.167700 | ESTs, Moderately similar to I38022 hypot | 3.00 | 3694 |
| 438703 | AI803373 | Hs.31599 | ESTs | 3.00 | 3159 |
| 451378 | BE153447 | | gb: PM2-HT0338-131299-001-b01 HT0338 Homo | 3.00 | 4217 |
| 444165 | AL137443 | Hs.10441 | hypothetical protein FLJ11236 | 3.00 | 3579 |
| 403532 | | | NM_024638: Homo sapiens hypothetical prot | 3.00 | 46 4715 |
| 453468 | W00712 | Hs.32990 | DKFZP566F084 protein | 3.00 | 4411 |
| 440278 | BE560870 | Hs.9052 | ESTs, Weakly similar to 2004399A chromos | 3.00 | 3276 |
| 456251 | R13326 | Hs.328475 | ESTs | 3.00 | 4534 |
| 425673 | R70318 | Hs.339730 | ESTs | 3.00 | 1985 |
| 442619 | AA447492 | Hs.20183 | ESTs, Weakly similar to AF164793 1 prote | 3.00 | 3455 |
| 459119 | AW844498 | Hs.306121 | CDC20 (cell division cycle 20, S. cerevi | 3.00 | 4657 |
| 455440 | AW946022 | | gb: QV0-ET0001-050500-229-b08 ET0001 Homo | 3.00 | 4508 |
| 413832 | AI913647 | Hs.131886 | Homo sapiens cDNA: FLJ22113 fis, clone H | 3.00 | 755 |
| 421515 | Y11339 | Hs.105352 | GalNAc alpha-2,6-sialyltransferase 1, l | 3.00 | 1532 5113 |
| 437994 | U92012 | Hs.251659 | ESTs, Weakly similar to mariner transpos | 3.00 | 3114 |
| 418827 | BE327311 | Hs.47166 | HT021 | 3.00 | 1245 |
| 420610 | AI683183 | Hs.99348 | distal-less homeo box 5 | 3.00 | 1437 |
| 401435 | | | C14000397*: gi\|7499898\|pir\|\|T33295 hypoth | 3.00 | 26 |
| 447072 | D61594 | Hs.17279 | tyrosylprotein sulfotransferase 1 | 3.00 | 3817 |
| 411975 | AI916058 | Hs.144583 | 3′UTR of: dead ringer (Drosophila)-like | 2.99 | 596 |
| 400965 | | | C11002190: gi\|12737279\|ref\|XP_012163.1\| | 2.99 | |
| 430594 | AK000790 | Hs.246885 | hypothetical protein FLJ20783 | 2.98 | 2498 5458 |
| 436469 | AK001455 | Hs.5198 | Down syndrome critical region gene 2 | 2.98 | 2986 |
| 421433 | AI829192 | Hs.22380 | ESTs | 2.98 | 1518 |
| 407756 | AA116021 | Hs.38260 | ubiquitin specific protease 18 | 2.98 | 191 |
| 433658 | L03678 | Hs.156110 | immunoglobulin kappa constant | 2.98 | 2778 5539 |
| 449969 | AW295142 | Hs.180187 | Homo sapiens cDNA FLJ14337 fis, clone PL | 2.98 | 4094 |
| 441962 | AW972542 | Hs.289008 | Homo sapiens cDNA: FLJ21814 fis, clone H | 2.97 | 3384 |
| 432241 | AI937060 | Hs.6298 | KIAA1151 protein | 2.97 | 2648 |
| 412560 | R24601 | | CCR4-NOT transcription complex, subunit | 2.97 | 645 |
| 433323 | AA805132 | Hs.159142 | ESTs | 2.97 | 2755 |
| 447444 | AK000318 | Hs.18616 | hypothetical protein FLJ20311 | 2.97 | 3862 5689 |
| 425465 | L18964 | Hs.1904 | protein kinase C, iota | 2.97 | 1969 5266 |
| 410337 | M83822 | Hs.62354 | cell division cycle 4-like | 2.97 | 465 4845 |
| 424800 | AL035588 | Hs.153203 | MyoD family inhibitor | 2.97 | 1888 5232 |
| 406709 | AI355761 | Hs.242463 | keratin 8 | 2.96 | 91 |
| 411412 | AJ001388 | Hs.69997 | zinc finger protein 238 | 2.96 | 4864 554 |
| 426761 | AI015709 | Hs.172089 | PORIMIN Pro-oncosis receptor inducing me | 2.96 | 2095 |
| 456564 | AW747802 | | gb: QV1-BN0003-061299-040-b09 BN0003 Homo | 2.96 | 4550 |
| 437273 | AL137451 | Hs.120873 | ESTs, Highly similar to T46266 hypotheti | 2.96 | 3053 5564 |
| 426820 | U73328 | Hs.172648 | distal-less homeobox 4 | 2.96 | 2102 5322 |
| 449209 | BE616830 | Hs.294145 | ESTs | 2.96 | 4046 |
| 402025 | | | NM_021624: Homo sapiens histamine H4 rece | 2.95 | 32 4702 |
| 416178 | AI808527 | Hs.192822 | serologically defined breast cancer anti | 2.95 | 978 |
| 446719 | W39500 | Hs.301872 | hypothetical protein MGC4840 | 2.95 | 3779 |
| 428500 | AI815395 | Hs.184641 | fatty acid desaturase 2 | 2.95 | 2266 |
| 417879 | R22128 | | gb: yh25h08.r1 Soares placenta Nb2HP Homo | 2.95 | 1134 |
| 404173 | | | Target Exon | 2.95 | |
| 404819 | | | NM_002688*: Homo sapiens peanut (Drosophi | 2.95 | 4728 62 |
| 448718 | AA220235 | Hs.153959 | hypothetical protein MGC15436 | 2.95 | 4000 |
| 402270 | | | Target Exon | 2.95 | |
| 420362 | U79734 | Hs.97206 | huntingtin interacting protein 1 | 2.95 | 1417 5075 |
| 451815 | AW974911 | Hs.184793 | Homo sapiens cDNA: FLJ21880 fis, clone H | 2.95 | 4250 |
| 445921 | AW015211 | Hs.146181 | ESTs | 2.95 | 3712 |
| 429412 | NM_006235 | Hs.2407 | POU domain, class 2, associating factor | 2.95 | 2365 5404 |
| 442875 | BE623003 | Hs.23625 | Homo sapiens clone TCCCTA00142 mRNA sequ | 2.95 | 3477 |
| 407804 | AF228603 | Hs.39957 | pleckstrin 2 (mouse) homolog | 2.95 | 197 4783 |
| 420569 | AA278362 | Hs.289062 | Homo sapiens cDNA FLJ12334 fis, clone MA | 2.95 | 1433 |
| 436389 | AI811706 | Hs.42733 | CHMP1.5 protein | 2.95 | 2981 |
| 424581 | M62062 | Hs.150917 | catenin (cadherin-associated protein), a | 2.95 | 1861 |
| 406749 | AW193917 | Hs.217493 | annexin A2 | 2.95 | 96 |
| 418295 | AW970043 | Hs.238039 | hypothetical protein FLJ11090 | 2.95 | 1177 |
| 431310 | AW327889 | Hs.252433 | Homo sapiens cDNA FLJ13794 fis, clone TH | 2.95 | 2545 |
| 434698 | BE044674 | | gb: hm46f02.x1 NCI_CGAP_RDF1 Homo sapiens | 2.95 | 2861 |
| 430316 | NM_000875 | Hs.239176 | insulin-like growth factor 1 receptor | 2.95 | 2470 5445 |
| 439210 | AA641928 | Hs.194071 | ESTs, Weakly similar to unnamed protein | 2.95 | 3192 |
| 414052 | AW578849 | Hs.283552 | ESTs, Weakly similar to unnamed protein | 2.95 | 779 |
| 418304 | AA215702 | | gb: zr97g10.r1 NCI_CGAP_GCB1 Homo sapiens | 2.95 | 1178 |
| 440327 | R12581 | Hs.191146 | ESTs | 2.94 | 3282 |
| 431707 | R21326 | Hs.267905 | hypothetical protein FLJ10422 | 2.94 | 2587 |
| 458898 | AW295097 | Hs.200260 | ESTs | 2.93 | 4644 |
| 408471 | NM_012317 | Hs.45231 | leucine zipper, down-regulated in cancer | 2.93 | 269 4797 |
| 418526 | BE019020 | Hs.85838 | solute carrier family 16 (monocarboxylic | 2.93 | 1211 |
| 446111 | W56338 | Hs.13880 | CGI-143 protein | 2.93 | 3725 |
| 410444 | W73464 | Hs.132554 | gb: zd54e04.s1 Soares_fetal_heart_NbHH19W | 2.93 | 480 |
| 439396 | BE562958 | Hs.74346 | hypothetical protein MGC14353 | 2.92 | 3205 |
| 433627 | AF078866 | Hs.284296 | Homo sapiens cDNA: FLJ22993 fis, clone K | 2.92 | 2774 5538 |
| 425683 | AB037813 | Hs.159200 | hypothetical protein DKFZp762K222 | 2.92 | 1987 5275 |
| 452480 | AI903526 | | gb: RC-BT031-090199-063 BT031 Homo sapien | 2.91 | 4314 |
| 458247 | R14439 | Hs.209194 | ESTs | 2.91 | 4617 |
| 410682 | H07103 | Hs.286014 | Homo sapiens, clone IMAGE: 3867243, mRNA | 2.91 | 505 |
| 445733 | BE295568 | Hs.13225 | UDP-Gal: betaGlcNAc beta 1,4-galactosylt | 2.91 | 3696 |
| 429986 | AF092047 | Hs.227277 | sine oculis homeobox (Drosophila) homolo | 2.91 | 2434 5431 |
| 419638 | N46504 | Hs.91747 | profilin 2 | 2.91 | 1341 |
| 414983 | L17128 | Hs.77719 | gamma-glutamyl carboxylase | 2.91 | 4937 897 |
| 442643 | U82756 | | PRP4/STK/WD splicing factor | 2.90 | 3457 5623 |
| 416361 | AW204907 | Hs.6872 | ESTs, Weakly similar to CA13_HUMAN COLLA | 2.90 | 989 |
| 437575 | AW954355 | Hs.36529 | hypothetical protein MGC11242 | 2.90 | 3069 |
| 431214 | AA294921 | Hs.348024 | v-ral simian leukemia viral oncogene hom | 2.90 | 2541 |
| 414064 | BE245289 | Hs.16165 | expressed in activated T/LAK lymphocytes | 2.90 | 784 |
| 446258 | AI283476 | Hs.263478 | ESTs | 2.90 | 3740 |
| 404607 | | | Target Exon | 2.90 | |
| 414094 | H15088 | Hs.31433 | ESTs | 2.90 | 787 |
| 443833 | AI654108 | Hs.135125 | ESTs | 2.90 | 3558 |
| 442712 | BE465168 | Hs.131011 | ESTs | 2.90 | 3461 |
| 411887 | AW182924 | Hs.128790 | ESTs | 2.90 | 584 |
| 405366 | | | NM_003371*: Homo sapiens vav 2 oncogene ( | 2.90 | 4735 69 |
| 404624 | | | karyopherin (importin) beta 3 | 2.90 | |
| 411387 | AW842339 | Hs.130815 | hypothetical protein FLJ21870 | 2.90 | 550 |
| 408990 | AL022395 | Hs.49526 | f-box and leucine-rich repeat protein 4 | 2.90 | 321 4810 |
| 417288 | AI984792 | Hs.108812 | hypothetical protein RLJ22004 | 2.90 | 1077 |
| 419465 | AW500239 | Hs.21187 | Homo sapiens cDNA: FLJ23068 fis, clone L | 2.90 | 1316 |
| 459010 | AA331438 | | gb: EST35269 Embryo, 8 week I Homo sapien | 2.90 | 4652 |
| 445008 | AW373754 | | gb: QV4-BT0536-291299-065-e01 BT0536 Homo | 2.90 | 3645 |
| 412170 | D16532 | Hs.73729 | very low density lipoprotein receptor | 2.90 | 4875 615 |
| 419752 | AA249573 | Hs.152618 | ESTs, Moderately similar to ZN91_HUMAN Z | 2.90 | 1358 |
| 419497 | NM_006410 | Hs.90753 | Tat-interacting protein (30 kD) | 2.90 | 1320 5050 |
| 423849 | AL157425 | Hs.133315 | Homo sapiens mRNA; cDNA DKFZp761J1324 (f | 2.90 | 1780 |
| 452909 | NM_015368 | Hs.30985 | pannexin 1 | 2.90 | 4358 5767 |
| 428560 | AI243209 | Hs.98669 | ESTs, Weakly similar to B47411 ADPribosy | 2.90 | 2270 |
| 413429 | BE139117 | Hs.278881 | ESTs | 2.90 | 719 |
| 426126 | AL118747 | Hs.26691 | AY046419: Homo sapiens glucose transport | 2.89 | 2030 |
| 409693 | AA010233 | Hs.55921 | glutamyl-prolyl-tRNA synthetase | 2.89 | 394 |
| 407242 | M18728 | | gb: Human nonspecific crossreacting antig | 2.89 | 142 4766 |
| 412174 | C04144 | Hs.172753 | ESTs | 2.89 | 616 |
| 447541 | AK000288 | Hs.18800 | hypothetical protein FLJ20281 | 2.88 | 3877 5691 |
| 409264 | NM_014937 | Hs.52463 | KIAA0966 protein | 2.88 | 356 4820 |
| 453063 | W47196 | Hs.166172 | aryl hydrocarbon receptor nuclear transl | 2.88 | 4371 |
| 431865 | AA521106 | Hs.136375 | ESTs, Weakly similar to S65824 reverse t | 2.88 | 2610 |
| 409231 | AA446644 | Hs.692 | GA733-2 antigen; epithelial glycoprotein | 2.88 | 353 |
| 424364 | AW383226 | Hs.163834 | ESTs, Weakly similar to G01763 atrophin- | 2.87 | 1835 |
| 408713 | NM_001248 | Hs.47042 | ectonucleoside triphosphate diphosphohyd | 2.87 | 296 4804 |
| 406972 | M32053 | | gb: Human H19 RNA gene, complete cds. | 2.87 | 115 |
| 404901 | | | NM_025213: Homo sapiens spectrin, beta, n | 2.87 | 4730 64 |
| 422939 | AW394055 | Hs.98427 | ESTs, Weakly similar to I38022 hypotheti | 2.87 | 1695 |
| 449824 | AI962552 | Hs.226765 | ESTs | 2.87 | 4086 |
| 409728 | AW883968 | Hs.321190 | ESTs | 2.87 | 401 |
| 418867 | D31771 | Hs.89404 | msh (Drosophila) homeo box homolog 2 | 2.87 | 1252 5032 |
| 429492 | W21183 | Hs.13205 | ESTs | 2.87 | 2378 |
| 429023 | NM_000312 | Hs.2351 | protein C (inactivator of coagulation fa | 2.86 | 2325 5387 |
| 416612 | H70565 | | gb: yr97c04.r1 Soares fetal liver spleen | 2.86 | 1007 |
| 440457 | BE387593 | Hs.21321 | Homo sapiens clone FLB9213 PRO2474 mRNA, | 2.86 | 3289 |
| 446620 | AA128808 | Hs.179902 | transporter-like protein | 2.86 | 3771 |
| 413517 | N76712 | Hs.44829 | ESTs, Weakly similar to I38022 hypotheti | 2.86 | 730 |
| 448475 | BE613134 | Hs.247474 | hypothetical protein FLJ21032 | 2.86 | 3973 |
| 433167 | AB037682 | Hs.195715 | hypothetical protein MGC4827 | 2.86 | 2744 5530 |
| 406137 | | | NM_000179*: Homo sapiens mutS (E. coli) h | 2.85 | 4742 76 |
| 457094 | AI360119 | Hs.181013 | phosphoglycerate mutase 1 (brain) | 2.85 | 4577 |
| 428792 | BE535955 | Hs.193602 | chromosome condensation protein G | 2.85 | 2300 |
| 410305 | AF030409 | Hs.62185 | solute carrier family 9 (sodium/hydrogen | 2.85 | 463 4843 |
| 408253 | AW807476 | Hs.21051 | Homo sapiens mRNA for FLJ00012 protein, | 2.85 | 250 |
| 430355 | NM_006219 | Hs.239818 | phosphoinositide-3-kinase, catalytic, be | 2.85 | 2475 5449 |
| 436854 | AA749167 | Hs.173911 | ESTs | 2.85 | 3010 |
| 412783 | BE276738 | Hs.74578 | DEAD/H (Asp-Glu-Ala-Asp/His) box polypep | 2.85 | 669 |
| 406303 | | | C16000922: gi\|7499103\|pir\|\|T20903 hypothe | 2.85 | 77 |
| 432917 | NM_014125 | Hs.241517 | PRO0327 protein | 2.85 | 2712 5518 |
| 430452 | AI888450 | Hs.174644 | hypothetical protein FLJ21669 | 2.85 | 2486 |
| 422805 | AA436989 | Hs.121017 | H2A histone family, member A | 2.85 | 1683 |
| 441642 | H85686 | Hs.7922 | HSPC154 protein | 2.85 | 3364 |
| 443614 | AV655386 | Hs.7645 | fibrinogen, B beta polypeptide | 2.85 | 3532 |
| 419270 | NM_005232 | Hs.89839 | EphA1 | 2.85 | 1295 5042 |
| 448807 | AI571940 | Hs.7549 | ESTs | 2.85 | 4009 |
| 413838 | AV661185 | Hs.75574 | mitochondrial ribosomal protein L19 | 2.85 | 757 |
| 412831 | AA121352 | Hs.143314 | ESTs | 2.85 | 676 |
| 429370 | C19097 | Hs.89709 | glutamate-cysteine ligase, modifier subu | 2.85 | 2361 |
| 438895 | AW007021 | Hs.82894 | ESTs | 2.85 | 3176 |
| 440516 | S42303 | Hs.161 | cadherin 2, type 1, N-cadherin (neuronal | 2.84 | 3294 5608 |
| 451721 | NM_006946 | Hs.26915 | spectrin, beta, non-erythrocytic 2 | 2.84 | 4242 5749 |
| 407813 | AL120247 | Hs.40109 | KIAA0872 protein | 2.84 | 200 |
| 417386 | AL037228 | Hs.82043 | D123 gene product | 2.84 | 1090 |
| 441021 | AW578716 | Hs.7644 | H1 histone family, member 2 | 2.84 | 3326 |
| 444184 | T87841 | Hs.282990 | Human DNA sequence from clone RP1-28H20 | 2.84 | 3581 |
| 416946 | NM_012324 | | mitogen-activated protein kinase 8 inter | 2.84 | 1041 4971 |
| 432734 | AA837396 | Hs.263925 | LIS1-interacting protein NUDE1, rat Homo | 2.84 | 2696 |
| 426779 | AA384577 | Hs.93714 | ESTs, Weakly similar to T00365 hypotheti | 2.84 | 2097 |
| 437679 | NM_014214 | Hs.5753 | inositol(myo)-1(or 4)-monophosphatase 2 | 2.84 | 3082 5590 |
| 427722 | AK000123 | Hs.180479 | hypothetical protein FLJ20116 | 2.84 | 2190 5344 |
| 423692 | BE621056 | Hs.131731 | hypothetical protein FLJ11099 | 2.83 | 1768 |
| 445654 | X91247 | Hs.13046 | thioredoxin reductase 1 | 2.83 | 3691 5658 |
| 415276 | U88666 | Hs.78353 | SFRS protein kinase 2 | 2.83 | 4945 922 |
| 429038 | AL023513 | Hs.194766 | seizure related gene 6 (mouse)-like | 2.83 | 2327 5388 |
| 432375 | BE536069 | Hs.2962 | S100 calcium-binding protein P | 2.82 | 2661 |
| 424481 | R19453 | Hs.1787 | proteolipid protein 1 (Pelizaeus-Merzbac | 2.82 | 1851 |
| 418027 | AB037807 | Hs.83293 | hypothetical protein | 2.82 | 1149 5000 |
| 429978 | AA249027 | | ribosomal protein S6 | 2.82 | 2433 |
| 421486 | AW408800 | Hs.104859 | hypothetical protein DKFZp762E1312 | 2.82 | 1525 |
| 436972 | AA284679 | Hs.25640 | claudin 3 | 2.82 | 3024 |
| 437786 | BE142681 | Hs.155573 | polymerase (DNA directed), eta | 2.81 | 3091 |
| 438881 | AA827691 | Hs.129967 | ESTs, Weakly similar to ALU1_HUMAN ALU S | 2.81 | 3175 |
| 408901 | AK001330 | Hs.48855 | hypothetical protein FLJ10468 | 2.81 | 316 4808 |
| 416984 | H38765 | Hs.80706 | diaphorase (NADH/NADPH) (cytochrome b-5 | 2.81 | 1047 |
| 422559 | AW247696 | Hs.155839 | hypothetical protein MGC12934 | 2.81 | 1656 |
| 422259 | AA307584 | | gb: EST178498 Colon carcinoma (HCC) cell | 2.81 | 1623 |
| 448789 | BE539108 | Hs.22051 | hypothetical protein MGC15548 | 2.81 | 4007 |
| 453164 | F33692 | Hs.32018 | SNARE associated protein snapin | 2.81 | 4381 |
| 414670 | BE409525 | Hs.902 | neurofibromin 2 (bilateral acoustic neur | 2.81 | 860 |
| 424971 | AA479005 | Hs.154036 | tumor suppressing subtransferable candid | 2.81 | 1903 |
| 417381 | AF164142 | Hs.82042 | solute carrier family 23 (nucleobase tra | 2.80 | 1089 4983 |
| 408681 | AW953853 | Hs.281462 | ESTs, Weakly similar to I38022 hypotheti | 2.80 | 293 |
| 459208 | BE261314 | Hs.250469 | ESTs, Weakly similar to I38022 hypotheti | 2.80 | 4659 |
| 445133 | AW157646 | Hs.198689 | ESTs | 2.80 | 3654 |
| 455236 | AW875972 | | gb: CM3-PT0014-071299-051-b05 PT0014 Homo | 2.80 | 4503 |
| 426647 | AA243464 | Hs.294101 | pre-B-cell leukemia transcription factor | 2.80 | 2085 |
| 411954 | AA652523 | Hs.269496 | ESTs, Weakly similar to I38022 hypotheti | 2.80 | 591 |
| 426925 | NM_001196 | Hs.315689 | Homo sapiens cDNA: FLJ22373 fis, clone H | 2.80 | 2112 5326 |
| 425128 | BE561929 | Hs.154718 | tumor protein D52-like 2 | 2.80 | 1924 |
| 432642 | BE297635 | Hs.3069 | heat shock 70 kD protein 9B (mortalin-2) | 2.80 | 2688 |
| 416128 | AA173632 | | CDC14 (cell division cycle 14, S. cerevi | 2.80 | 974 |
| 427342 | AL110150 | Hs.176680 | Homo sapiens mRNA; cDNA DKFZp586D0724 (f | 2.80 | 2144 |
| 419544 | AI909154 | | gb: QV-BT200-010499-007 BT200 Homo sapien | 2.80 | 1329 |
| 450214 | BE439763 | Hs.227571 | regulator of G-protein signalling 4 | 2.80 | 4113 |
| 403848 | | | arylsulfatase E (chondrodysplasia puncta | 2.80 | |
| 448002 | Y15227 | Hs.20149 | deleted in lymphocytic leukemia, 1 | 2.80 | 3935 5705 |
| 431999 | AL133573 | Hs.272312 | Homo sapiens mRNA; cDNA DKFZp434J2235 (f | 2.80 | 2625 |
| 402261 | | | C19001033*: gi\|6754398\|ref\|NP_036071.1\|J | 2.80 | |
| 415660 | AI909007 | Hs.78563 | ubiquitin-conjugating enzyme E2G 1 (Homo | 2.80 | 943 |
| 428134 | AA421773 | Hs.161008 | ESTs | 2.80 | 2221 |
| 437147 | AL049964 | | hypothetical protein FLJ20366 | 2.80 | 3037 |
| 442562 | BE379584 | | dolichyl-diphosphooligosaccharide-protei | 2.80 | 3443 |
| 423441 | R68649 | Hs.278359 | absent in melanoma 1 like | 2.80 | 1745 |
| 436211 | AK001581 | Hs.334828 | hypothetical protein FLJ10719; KIAA1794 | 2.80 | 2967 5576 |
| 437341 | AA749062 | | ESTs, Weakly similar to I54374 gene NF2 | 2.80 | 3059 |
| 454453 | AW752781 | | hypothetical protein FLJ12614 similar to | 2.80 | 4485 |
| 434723 | W17044 | Hs.327337 | ESTs | 2.79 | 2862 |
| 442553 | H87867 | Hs.40065 | hypothetical protein MGC4825 | 2.79 | 3440 |
| 432728 | NM_006979 | Hs.278721 | HLA class II region expressed gene KE4 | 2.79 | 2694 5512 |
| 409132 | AJ224538 | Hs.50732 | protein kinase, AMP-activated, beta 2 no | 2.79 | 338 4813 |
| 453863 | X02544 | Hs.572 | orosomucoid 1 | 2.79 | 4438 5786 |
| 447604 | AW089933 | Hs.301342 | hypothetical protein MGC4342 | 2.79 | 3885 |
| 432195 | AJ243669 | Hs.8127 | KIAA0144 gene product | 2.79 | 2642 5503 |
| 448850 | AB018286 | Hs.247837 | neurexin 3 | 2.79 | 4013 5715 |
| 445800 | AA126419 | Hs.32944 | inositol polyphosphate-4-phosphatase, ty | 2.79 | 3699 |
| 449019 | AI949095 | Hs.67776 | ESTs, Weakly similar to T22341 hypotheti | 2.78 | 4028 |
| 452747 | BE153855 | Hs.61460 | Ig superfamily receptor LNIR | 2.78 | 4341 |
| 409190 | AU076536 | Hs.50984 | sarcoma amplified sequence | 2.78 | 349 |
| 446080 | AI221741 | Hs.117777 | ESTs | 2.78 | 3723 |
| 442476 | AF069475 | | gb: AF069475 Homo sapiens astrocytoma lib | 2.78 | 3433 |
| 414907 | X90725 | Hs.77597 | polo (Drosophia)-like kinase | 2.78 | 4933 891 |
| 451150 | AI888124 | Hs.285280 | Homo sapiens cDNA: FLJ22096 fis, clone H | 2.78 | 4197 |
| 438182 | AW342140 | Hs.182545 | ESTs, Weakly similar to ALU1_HUMAN ALU S | 2.78 | 3124 |
| 420164 | AW339037 | Hs.24908 | ESTs | 2.78 | 1390 |
| 422404 | AL133571 | Hs.336189 | Homo sapiens mRNA: cDNA DKFZp434F1135 (f | 2.77 | 1639 |
| 451958 | AW207328 | | ESTs | 2.77 | 4259 |
| 444895 | AI674383 | Hs.22891 | solute carrier family 7 (cationic amino | 2.76 | 3638 |
| 421260 | N46574 | Hs.43838 | ESTs | 2.76 | 1499 |
| 443507 | AI769289 | Hs.22383 | hypothetical protein FLJ11336 | 2.76 | 3525 |
| 446383 | T05816 | Hs.92511 | ESTs | 2.76 | 3748 |
| 424118 | BE269041 | Hs.140452 | cargo selection protein (mannose 6 phosp | 2.76 | 1805 |
| 407699 | AA825974 | Hs.32646 | hypothetical protein FLJ21901 | 2.76 | 182 |
| 422170 | AI791949 | Hs.112432 | anti-Mullerian hormone | 2.76 | 1613 |
| 400294 | N95796 | Hs.278695 | Homo sapiens prostein mRNA, complete cds | 2.76 | 7 |
| 441181 | AA416925 | | peptidylprolyl isomerase (cyclophilin)-I | 2.76 | 3337 |
| 415889 | R24563 | | VPS10 domain receptor protein | 2.75 | 957 |
| 400821 | | | Target Exon | 2.75 | |
| 416814 | AW192307 | Hs.80042 | dolichyl-P-Glc: Man9GlcNAc2-PP-dolichylgl | 2.75 | 1026 |
| 400857 | | | ENSP00000228038*: PUTATIVE SERINE/THREONI | 2.75 | |
| 411666 | AF106564 | Hs.71346 | neurofilament 3 (150 kD medium) | 2.75 | 566 |
| 444185 | AW298350 | Hs.113602 | ESTs | 2.75 | 3582 |
| 443385 | AI792839 | Hs.133113 | ESTs | 2.75 | 3521 |
| 445873 | AA250970 | Hs.251946 | poly(A)-binding protein, cytoplasmic 1-I | 2.75 | 3707 |
| 441102 | AA973905 | | intermediate filament protein syncoilin | 2.75 | 3333 |
| 411630 | U42349 | Hs.71119 | Putative prostate cancer tumor suppresso | 2.75 | 4866 565 |
| 456193 | H43437 | Hs.80305 | hypothetical protein MGC14258 | 2.75 | 4531 |
| 421342 | AA504749 | | gb: aa63f08.r1 NCI_CGAP_GCB1 Homo sapiens | 2.75 | 1510 |
| 419507 | AW170425 | Hs.87680 | ESTs | 2.75 | 1322 |
| 439602 | W79114 | Hs.58558 | ESTs | 2.75 | 3222 |
| 453775 | NM_002916 | Hs.35120 | replication factor C (activator 1) 4 (37 | 2.75 | 4431 5783 |
| 438054 | AA776626 | Hs.169309 | ESTs | 2.75 | 3115 |
| 423598 | BE247600 | Hs.155538 | ESTs | 2.75 | 1760 |
| 407771 | AL138272 | Hs.1600 | ESTs | 2.75 | 193 |
| 423492 | AF020761 | Hs.129683 | ubiquitin-conjugating enzyme E2D 1 (Homo | 2.75 | 1752 |
| 422336 | AI761322 | Hs.115285 | dihydrolipoamide S-acetyltransferase (E2 | 2.75 | 1633 |
| 409717 | AW452871 | Hs.56043 | CGI-115 protein | 2.75 | 400 |
| 447818 | W79940 | Hs.21906 | Homo sapiens clone 24670 mRNA sequence | 2.75 | 3909 |
| 414361 | AI086138 | Hs.204044 | ESTs | 2.75 | 816 |
| 424840 | D79987 | Hs.153479 | extra spindle poles, S. cerevisiae, Homo | 2.75 | 1890 5233 |
| 442711 | AF151073 | Hs.8645 | hypothetical protein | 2.75 | 3460 5625 |
| 436561 | BE560135 | Hs.5232 | HSPC125 protein | 2.75 | 2994 |
| 408576 | NM_003542 | Hs.46423 | H4 histone family, member G | 2.74 | 280 4800 |
| 443724 | AA446783 | Hs.288932 | hypothetical protein FLJ13194 | 2.74 | 3546 |
| 441057 | AL043897 | | ESTs | 2.74 | 3330 |
| 450983 | AA305384 | Hs.25740 | ERO1 (S. cerevisiae)-like | 2.73 | 4180 |
| 420230 | AL034344 | Hs.284186 | forkhead box C1 | 2.73 | 1398 5069 |
| 424873 | AB018294 | Hs.153610 | KIAA0751 gene product | 2.73 | 1893 5235 |
| 452960 | AK001335 | Hs.31137 | protein tyrosine phosphatase, receptor t | 2.73 | 4360 |
| 442861 | AA243837 | Hs.57787 | ESTs | 2.73 | 3475 |
| 416801 | X98834 | Hs.79971 | sal (Drosophila)-like 2 | 2.73 | 1024 |
| 423250 | BE061916 | Hs.125849 | chromosome 8 open reading frame 2 | 2.73 | 1732 |
| 416959 | D28459 | Hs.80612 | ubiquitin-conjugating enzyme E2A (RAD6 h | 2.73 | 1043 4973 |
| 413719 | BE439580 | Hs.75498 | small inducible cytokine subfamily A (Cy | 2.73 | 749 |
| 446100 | AW967109 | Hs.13804 | hypothetical protein dJ462O23.2 | 2.73 | 3724 |
| 442404 | AI733500 | Hs.124370 | ESTs | 2.73 | 3426 |
| 412988 | BE046680 | | gb: hn42h03.x1 NCI_CGAP_RDF2 Homo sapiens | 2.72 | |
| 421582 | AI910275 | | trefoil factor 1 (breast cancer, estroge | 2.72 | 1541 |
| 434288 | AW189075 | Hs.116265 | fibrillin3 | 2.72 | 2826 |
| 418461 | BE242781 | Hs.102971 | hypothetical protein FLJ14751 | 2.72 | 1203 |
| 454462 | AW754153 | Hs.155976 | cullin 4B | 2.72 | 4486 |
| 451089 | AA903705 | Hs.4190 | Homo sapiens cDNA: FLJ23269 fis, clone C | 2.72 | 4191 |
| 441436 | AW137772 | Hs.185980 | ESTs | 2.71 | 3353 |
| 430227 | AI924441 | Hs.236218 | TAT-INTERACTIVE PROTEIN, 72-KD | 2.71 | 2456 |
| 417089 | H52280 | Hs.18612 | Homo sapiens cDNA: FLJ21909 fis, clone H | 2.71 | 1062 |
| 403671 | | | C4001270*: gi\|7509005\|pir\|\|T26190 hypothe | 2.71 | 47 |
| 436895 | AF037335 | Hs.5338 | carbonic anhydrase XII | 2.71 | 3019 5582 |
| 435474 | W92102 | Hs.117269 | ESTs | 2.71 | 2918 |
| 408405 | AK001332 | Hs.44672 | hypothetical protein FLJ10470 | 2.71 | 264 4796 |
| 420319 | AW406289 | Hs.96593 | hypothetical protein | 2.70 | 1410 |
| 419329 | AY007220 | Hs.288998 | S100-type calcium binding protein A14 | 2.70 | 1301 5043 |
| 402280 | | | C19002023: gi\|12741355ref\|XP_012768.1\|z | 2.70 | |
| 415208 | F01020 | Hs.172004 | titin | 2.70 | 916 |
| 400240 | | | Eos Control | 2.70 | |
| 405976 | | | Target Exon | 2.70 | |
| 400074 | | | Eos Control | 2.70 | |
| 432093 | H28383 | | gb: yl52c03.r1 Soares breast 3NbHBst Homo | 2.70 | 2630 |
| 435579 | AI332373 | Hs.156924 | ESTs | 2.70 | 2930 |
| 443323 | BE560621 | Hs.9222 | estrogen receptor binding site associate | 2.70 | 3518 |
| 412505 | AA974491 | Hs.21734 | ESTs | 2.70 | 638 |
| 451311 | D61040 | | gb: HUM149E10B Clontech human fetal brain | 2.70 | 4208 |
| 437025 | AW296618 | Hs.120637 | ESTs | 2.70 | 3028 |
| 414429 | R51494 | Hs.71818 | ESTs | 2.70 | 827 |
| 432188 | AI362952 | Hs.2928 | solute carrier family 7 (cationic amino | 2.70 | 2641 |
| 457873 | AA736920 | Hs.288518 | ESTs | 2.70 | 4603 |
| 455290 | U75810 | | gb: HSU75810 Human Homo sapiens cDNA clon | 2.70 | 4505 |
| 447678 | BE385257 | Hs.63908 | Homo sapiens dopamine receptor interacti | 2.70 | 3890 |
| 429782 | NM_005754 | Hs.220689 | Ras-GTPase-activating protein SH3-domain | 2.70 | 2416 5425 |
| 406203 | | | Target Exon | 2.70 | |
| 419030 | T79957 | Hs.188466 | ESTs | 2.70 | 1268 |
| 444911 | U06117 | Hs.250 | xanthene dehydrogenase | 2.70 | 3639 5647 |
| 456759 | BE259150 | Hs.127792 | delta (Drosophila)-like 3 | 2.70 | 4560 |
| 445564 | AB028957 | Hs.12896 | KIAA1034 protein | 2.70 | 3684 5656 |
| 428816 | AA004986 | Hs.193852 | ATP-binding cassette, sub-family C (CFTR | 2.70 | 2303 |
| 416800 | H89652 | | gb: yu81f05.r1 Soares fetal liver spleen | 2.70 | 1023 |
| 418104 | T05726 | Hs.177130 | ESTs | 2.70 | 1159 |
| 409435 | AI810721 | Hs.95424 | ESTs | 2.70 | 370 |
| 412926 | AI879076 | Hs.75061 | macrophage myristoylated alanine-rich C | 2.70 | 682 |
| 436876 | AI124756 | Hs.5337 | isocitrate dehydrogenase 2 (NADP), mitoc | 2.70 | 3016 |
| 447400 | AK000322 | Hs.18457 | hypothetical protein FLJ20315 | 2.69 | 3857 5686 |
| 452436 | BE077546 | Hs.31447 | ESTs, Moderately similar to A46010 X-lin | 2.69 | 4308 |
| 409775 | AW499605 | | gb: UI-HF-BP0p-ain-c-05-0-UI.r1 NIH_MGC_5 | 2.69 | 404 |
| 450222 | U75308 | Hs.24644 | TATA box binding protein (TBP)-associate | 2.69 | 4115 5726 |
| 414416 | AW409985 | Hs.76084 | hypothetical protein MGC2721 | 2.69 | 824 |
| 452567 | D87120 | Hs.29882 | predicted osteoblast protein | 2.69 | 4326 5759 |
| 421485 | AA243499 | Hs.104800 | hypothetical protein FLJ10134 | 2.69 | 1524 |
| 402609 | | | KIAA1209 protein | 2.69 | |
| 419741 | NM_007019 | Hs.93002 | ubiquitin carrier protein E2-C | 2.68 | 1355 5056 |
| 417715 | AW969587 | Hs.86366 | ESTs | 2.68 | 1115 |
| 411669 | BE612676 | Hs.303116 | stromal cell-derived factor 2-like 1 | 2.68 | 567 |
| 439424 | AI478667 | Hs.118183 | hypothetical protein FLJ22833 | 2.68 | 3207 |
| 409590 | AW997069 | Hs.99010 | hypothetical protein FLJ22263 similar to | 2.68 | 386 |
| 402927 | | | ENSP00000243914*: DJ579F20.2 (similar to | 2.68 | |
| 409529 | AB007884 | Hs.54697 | Cdc42 guanine exchange factor (GEF) 9 | 2.68 | 381 4826 |
| 450816 | BE271927 | Hs.87385 | ESTs | 2.68 | 4168 |
| 416221 | BE513171 | Hs.79086 | mitochondrial ribosomal protein L3 | 2.67 | 982 |
| 427505 | AA361562 | Hs.178761 | 26S proteasome-associated pad1 homolog | 2.67 | 2158 |
| 424629 | M90656 | Hs.151393 | glutamate-cysteine ligase, catalytic sub | 2.67 | 1865 5221 |
| 428844 | AW972635 | Hs.301904 | hypothetical protein FLJ12671 | 2.67 | 2311 |
| 453464 | AI884911 | Hs.32989 | receptor (calcitonin) activity modifying | 2.67 | 4409 |
| 422424 | AI186431 | Hs.296638 | prostate differentiation factor | 2.67 | 1645 |
| 410703 | AW798089 | | gb: CM0-UM0045-030300-262-c05 UM0045 Homo | 2.67 | 509 |
| 418385 | AW590613 | Hs.301040 | Homo sapiens, clone IMAGE: 3357127, mRNA, | 2.67 | 1194 |
| 430301 | AI902657 | Hs.188662 | KIAA1702 protein | 2.67 | 2466 |
| 403324 | | | C2000428*: gi\|7705383\|ref\|NP_057536.1\|GC | 2.67 | |
| 430532 | D61216 | Hs.18672 | ESTs | 2.67 | 2494 |
| 426153 | AF057169 | Hs.182771 | vitelliform macular dystrophy (Best dise | 2.67 | 2032 5293 |
| 435602 | AF217515 | Hs.283532 | uncharacterized bone marrow protein BM03 | 2.67 | 2931 5567 |
| 450669 | AL138077 | Hs.16157 | hypothetical protein FLJ12707 | 2.67 | 4146 |
| 450587 | AI828854 | Hs.258538 | striatin, calmodulin-binding protein | 2.67 | 4142 |
| 452256 | AK000933 | Hs.28661 | Homo sapiens cDNA FLJ10071 fis, clone HE | 2.67 | 4289 |
| 446525 | AW967069 | Hs.211556 | hypothetical protein MGC5487 | 2.67 | 3758 |
| 450273 | AW296454 | Hs.24743 | hypothetical protein FLJ20171 | 2.66 | 4121 |
| 409361 | NM_005982 | Hs.54416 | sine oculis homeobox (Drosophila) homolo | 2.66 | 364 4823 |
| 452826 | BE245286 | Hs.301636 | peroxisomal biogenesis factor 6 | 2.66 | 4349 |
| 429500 | X78565 | Hs.289114 | hexabrachion (tenascin C, cytotactin) | 2.66 | 2380 5410 |
| 444476 | AF020038 | Hs.11223 | isocitrate dehydrogenase 1 (NADP), solub | 2.66 | 3601 5639 |
| 400528 | | | NM_020975*: Homo sapiens ret proto-oncoge | 2.66 | 18 4690 |
| 423445 | NM_014324 | Hs.128749 | alpha-methylacyl-CoA racemase | 2.66 | 1746 5179 |
| 419704 | AA429104 | Hs.45057 | ESTs | 2.66 | 1350 |
| 442923 | AW248322 | Hs.95835 | ESTs, Weakly similar to unnamed protein | 2.66 | 3482 |
| 445786 | AW629819 | Hs.144502 | hypothetical protein FLJ22055 | 2.66 | 3697 |
| 445707 | AI248720 | Hs.114390 | ESTs | 2.66 | 3695 |
| 410575 | BE207480 | Hs.6994 | Homo sapiens cDNA: FLJ22044 fis, clone H | 2.65 | 494 |
| 435327 | BE301871 | Hs.4867 | mannosyl (alpha-1,3-)-glycoprotein beta- | 2.65 | 2908 |
| 428788 | AF082283 | Hs.193516 | B-cell CLL/lymphoma 10 | 2.65 | 2299 5378 |
| 447776 | AI525625 | Hs.130181 | UDP-N-acetyl-alpha-D-galactosamine: polyp | 2.65 | 3905 |
| 442604 | BE263710 | | gb: 601192150F1 NIH_MGC_7 Homo sapiens cD | 2.65 | 3451 |
| 445572 | AI243445 | Hs.189654 | ESTs | 2.65 | 3687 |
| 436581 | AA725726 | Hs.180213 | ESTs | 2.65 | 2995 |
| 450671 | AI356967 | Hs.43086 | ESTs, Weakly similar to A46010 X-linked | 2.65 | 4147 |
| 424238 | AA337401 | Hs.137635 | ESTs | 2.65 | 1817 |
| 404478 | | | NM_015435: Homo sapiens double ring-finge | 2.65 | 4722 55 |
| 402265 | | | Target Exon | 2.65 | |
| 414872 | U82010 | Hs.77513 | COX10 (yeast) homolog, cytochrome c oxid | 2.65 | 4932 886 |
| 450927 | AI807804 | Hs.134342 | TASP for testis-specific adriamycin sens | 2.65 | 4175 |
| 447673 | AI823987 | Hs.182285 | ESTs | 2.65 | 3889 |
| 424686 | AA345504 | | gb: EST51529 Gall bladder II Homo sapiens | 2.65 | 1873 |
| 420210 | AI557257 | Hs.44811 | ESTs | 2.65 | 1395 |
| 452451 | AA311250 | Hs.5621 | ESTs, Weakly similar to A46010 X-linked | 2.65 | 4309 |
| 449570 | AA001793 | | gb: zh86c06.r1 Soares_fetal_liver_spleen_{—} | 2.65 | 4068 |
| 410252 | AW821182 | Hs.61418 | microfibrillar-associated protein 1 | 2.65 | 460 |
| 428186 | AW504300 | Hs.295605 | mannosidase, alpha, class 2A, member 2 | 2.65 | 2228 |
| 453878 | AW964440 | Hs.19025 | DC32 | 2.65 | 4442 |
| 435274 | AA887547 | Hs.150905 | ESTs | 2.65 | 2905 |
| 430001 | AI580056 | Hs.98992 | ESTs | 2.65 | 2436 |
| 401796 | | | NM_021213*: Homo sapiens phosphatidylchol | 2.65 | 29 4699 |
| 401783 | | | NM_003771*: Homo sapiens keratin, hair, a | 2.65 | 28 4698 |
| 419362 | N64116 | Hs.24624 | hypothetical protein FLJ21945 | 2.65 | 1306 |
| 455737 | BE072246 | | gb: QV4-BT0536-271299-059-b12 BT0536 Homo | 2.65 | 4520 |
| 408161 | AW952912 | Hs.300383 | hypothetical protein MGC3032 | 2.65 | 240 |
| 436488 | BE620909 | Hs.261023 | hypothetical protein FLJ20958 | 2.65 | 2989 |
| 424736 | AF230877 | Hs.152701 | microtubule-interacting protein that ass | 2.65 | 1880 5230 |
| 444656 | AI277924 | Hs.145199 | ESTs | 2.65 | 3617 |
| 422516 | BE258862 | Hs.117950 | multifunctional polypeptide similar to S | 2.65 | 1652 |
| 431070 | AW408164 | Hs.249184 | transcription factor 19 (SC1) | 2.65 | 2529 |
| 418166 | AI754416 | | Cdc42 effector protein 3 | 2.65 | 1166 |
| 402553 | | | C1001756*: gi\|2143875\|pir\|\|I73636 neurona | 2.65 | 35 |
| 447587 | AW292139 | Hs.115789 | ESTs | 2.64 | 3884 |
| 424339 | BE257148 | | endoglycan | 2.64 | 1831 |
| 424723 | BE409813 | Hs.152337 | protein arginine N-methyltransferase 3(h | 2.64 | 1878 |
| 431842 | NM_005764 | Hs.271473 | epithelial protein up-regulated in carci | 2.64 | 2604 5492 |
| 445101 | T75202 | Hs.12314 | Homo sapiens mRNA; cDNA DKFZp586C1019 (f | 2.64 | 3652 |
| 419234 | AI565714 | Hs.101660 | ESTs | 2.64 | 1291 |
| 429617 | X89984 | Hs.211563 | B-cell CLL/lymphoma 7A | 2.64 | 2397 5417 |
| 458513 | AI138322 | Hs.154894 | ESTs | 2.63 | 4627 |
| 422872 | BE326786 | Hs.187646 | ESTs | 2.63 | 1688 |
| 433980 | AA137152 | Hs.286049 | phosphoserine aminotransferase | 2.63 | 2803 |
| 458692 | BE549905 | Hs.231754 | ESTs | 2.63 | 4633 |
| 435607 | W73428 | Hs.8750 | uncharacterized bone marrow protein BM04 | 2.63 | 2932 |
| 412141 | AI183838 | Hs.48938 | hypothetical protein FLJ21802 | 2.63 | 614 |
| 448857 | BE264467 | Hs.117929 | ESTs | 2.63 | 4015 |
| 441616 | BE569122 | | RNA-binding protein (autoantigenic) | 2.63 | 3360 |
| 438472 | AW974907 | Hs.86228 | TRIAD3 protein | 2.62 | 3142 |
| 415214 | AI445236 | Hs.125124 | EphB2 | 2.62 | 917 |
| 421810 | AK001718 | Hs.108530 | hypothetical protein FLJ10856 | 2.62 | 1566 5123 |
| 434811 | AW971205 | Hs.114280 | ESTs | 2.62 | 2869 |
| 423081 | AF262992 | Hs.123159 | sperm associated antigen 4 | 2.62 | 1717 5167 |
| 418655 | AA226354 | Hs.111240 | ESTs | 2.62 | 1220 |
| 412760 | AW379030 | Hs.41324 | ESTs | 2.62 | 668 |
| 417848 | AA206581 | Hs.116586 | ESTs, Weakly similar to JC5314 CDC28/cdc | 2.62 | 1129 |
| 405558 | | | Target Exon | 2.61 | |
| 423450 | AJ290445 | Hs.128759 | KIAA0524 protein | 2.61 | 1747 5180 |
| 420071 | AB028985 | Hs.94806 | ATP-binding cassette, sub-family A (ABC1 | 2.61 | 1380 5063 |
| 430654 | AW970081 | Hs.325603 | ESTs | 2.61 | 2503 |
| 412756 | N27422 | | ESTs, Moderately similar to Z195_HUMAN Z | 2.61 | 667 |
| 429045 | AA443751 | Hs.191819 | ESTs, Weakly similar to I38022 hypotheti | 2.60 | 2328 |
| 453392 | U23752 | Hs.32964 | SRY (sex determining region Y)-box 11 | 2.60 | 4403 5776 |
| 404576 | | | Target Exon | 2.60 | |
| 426028 | NM_001110 | Hs.172028 | a disintegrin and metalloproteinase doma | 2.60 | 2020 5287 |
| 451862 | H09260 | Hs.32333 | ESTs | 2.60 | 4254 |
| 416505 | H66470 | Hs.16004 | ESTs | 2.60 | 1000 |
| 417807 | R17806 | Hs.269452 | gb: yg09b06.r1 Soares infant brain 1NIB H | 2.60 | 1125 |
| 418105 | AW937488 | Hs.246381 | ESTs, Weakly similar to FV1 MOUSE FRIEND | 2.60 | 1160 |
| 401924 | | | ENSP00000246632*: CDNA FLJ20261 fis, clon | 2.60 | |
| 436027 | AI864053 | Hs.39972 | GM2 ganglioside activator protein (GM2A) | 2.60 | 2956 |
| 451446 | AI826288 | Hs.171637 | hypothetical protein MGC2628 | 2.60 | 4223 |
| 406389 | | | C5001408*: gi\|12621134\|ref\|NP_075244.1\|M | 2.60 | |
| 433906 | AI167816 | Hs.43355 | ESTs | 2.60 | 2796 |
| 404156 | | | C6002456: gi\|6755268\|ref\|NP_036008.1\|RAS | 2.60 | |
| 418771 | AA807881 | Hs.172792 | ESTs | 2.60 | 1239 |
| 420270 | AA257990 | Hs.137427 | ESTs | 2.60 | 1404 |
| 445371 | AI221645 | Hs.147800 | ESTs | 2.60 | 3673 |
| 439453 | BE264974 | Hs.6566 | thyroid hormone receptor interactor 13 | 2.60 | 3208 |
| 415857 | AA866115 | Hs.127797 | Homo sapiens cDNA FLJ11381 fis, clone HE | 2.60 | 956 |
| 445326 | AI220072 | | ESTs | 2.60 | 3669 |
| 416921 | N20177 | Hs.205024 | ESTs, Highly similar to B34087 hypotheti | 2.60 | 1038 |
| 432723 | D29677 | Hs.3085 | KIAA0054 gene product: Helicase | 2.60 | 2693 |
| 408031 | AA081395 | Hs.42173 | Homo sapiens cDNA FLJ10366 fis, clone NT | 2.60 | 225 |
| 431567 | N51357 | Hs.260855 | Homo sapiens cDNA: FLJ21410 fis, clone C | 2.60 | 2569 |
| 417183 | R52089 | Hs.172717 | ESTs | 2.60 | 1069 |
| 448663 | BE614599 | | hypothetical protein MGC14797 | 2.60 | 3993 |
| 430393 | BE185030 | Hs.241305 | estrogen-responsive B box protein | 2.60 | 2480 |
| 407325 | AA291180 | Hs.328476 | ESTs, Weakly similar to alternatively sp | 2.60 | 153 |
| 430408 | AA478540 | Hs.108336 | ESTs, Weakly similar to ALUE_HUMAN !!!! | 2.59 | 2483 |
| 418838 | AW385224 | Hs.35198 | ectonucleotide pyrophosphatase/phosphodi | 2.59 | 1248 |
| 409937 | AI804584 | Hs.57672 | leucine rich repeat (in FLII) interactin | 2.59 | 421 |
| 446937 | AA977190 | | ESTs | 2.59 | 3803 |
| 439343 | AF086161 | Hs.114611 | hypothetical protein FLJ11808 | 2.59 | 3201 |
| 434449 | AW953484 | Hs.3849 | hypothetical protein FLJ22041 similar to | 2.59 | 2841 |
| 456161 | BE264645 | Hs.282093 | hypothetical protein FLJ21918 | 2.59 | 4530 |
| 429294 | AA095971 | Hs.198793 | Homo sapiens cDNA: FLJ22463 fis, clone H | 2.59 | 2351 |
| 423357 | AI285124 | | ESTs | 2.59 | 1738 |
| 410359 | R38624 | Hs.106313 | ESTs | 2.59 | 468 |
| 426357 | AW753757 | Hs.12396 | gb: RC3-CT0283-271099-021-a08 CT0283 Homo | 2.59 | 2058 |
| 450378 | AW249181 | Hs.198899 | ESTs, Weakly similar to T19873 hypotheti | 2.58 | 4135 |
| 409710 | C04706 | Hs.42733 | CHMP1.5 protein | 2.58 | 398 |
| 452526 | W38537 | Hs.280740 | hypothetical protein MGC3040 | 2.58 | 4322 |
| 414774 | X02419 | Hs.77274 | plasminogen activator, urokinase | 2.58 | 4928 877 |
| 432435 | BE218886 | Hs.282070 | ESTs | 2.58 | 2667 |
| 448069 | U76248 | Hs.20191 | seven in absentia (Drosophila) homolog 2 | 2.58 | 3939 5707 |
| 423279 | AW959861 | Hs.290943 | ESTs | 2.58 | 1733 |
| 428708 | NM_014897 | Hs.190386 | KIAA0924 protein | 2.58 | 2284 5371 |
| 432026 | AA524545 | Hs.224630 | ESTs | 2.57 | 2627 |
| 447333 | BE090580 | Hs.70704 | hypothetical protein dJ616B8.3 | 2.57 | 3843 |
| 426272 | AW450671 | Hs.189284 | ESTs | 2.57 | 2046 |
| 447380 | AA743756 | Hs.116695 | ESTs, Weakly similar to I38022 hypotheti | 2.57 | 3852 |
| 407881 | AW072003 | Hs.40968 | heparan sulfate (glucosamine) 3-O-sulfot | 2.57 | 211 |
| 456906 | AF117646 | Hs.156637 | Cau-Br-M (murine) ectropic retroviral tr | 2.57 | 4567 5804 |
| 416065 | BE267931 | Hs.78996 | proliferating cell nuclear antigen | 2.57 | 969 |
| 407590 | AI831258 | | ESTs | 2.57 | 171 |
| 429170 | NM_001394 | Hs.2359 | dual specificity phosphatase 4 | 2.57 | 2336 5392 |
| 408246 | N55669 | Hs.333823 | mitochondrial ribosomal protein L13 | 2.57 | 248 |
| 440660 | AI300101 | Hs.131886 | Homo sapiens cDNA: FLJ22113 fis, clone H | 2.56 | 3310 |
| 422630 | AA313606 | Hs.13809 | hypothetical protein FLJ10648 | 2.56 | 1664 |
| 410913 | AL050367 | Hs.66762 | Homo sapiens mRNA; cDNA DKFZp564A026 (fr | 2.56 | 4859 520 |
| 456940 | H46986 | Hs.31861 | ESTs | 2.56 | 4569 |
| 429588 | AI080271 | | ESTs | 2.56 | 2391 |
| 427227 | AF103803 | Hs.283690 | hypothetical protein | 2.56 | 2133 5334 |
| 447347 | AA570056 | Hs.122730 | ESTs, Moderately similar to KIAA1215 pro | 2.56 | 3847 |
| 411423 | AW845987 | Hs.68864 | ESTs, Weakly similar to phosphatidylseri | 2.56 | 555 |
| 437175 | AW968078 | Hs.87773 | protein kinase, cAMP-dependent, catalyti | 2.56 | 3041 |
| 445652 | AL117473 | Hs.13036 | DKFZP727A071 protein | 2.56 | 3690 5657 |
| 422421 | AA325138 | Hs.235873 | hypothetical protein FLJ22672 | 2.56 | 1644 |
| 440115 | R41808 | Hs.144924 | ESTs, Weakly similar to B Chain B, Solut | 2.55 | 3265 |
| 421508 | NM_004833 | Hs.105115 | absent in melanoma 2 | 2.55 | 1529 5111 |
| 447532 | AK000614 | Hs.18791 | hypothetical protein FLJ20607 | 2.55 | 3875 5690 |
| 404580 | | | trichorhinophalangeal syndrome I gene (T | 2.55 | |
| 444454 | BE018316 | Hs.11183 | sorting nexin 2 | 2.55 | 3599 |
| 421557 | AA292875 | Hs.97333 | ESTs | 2.55 | 1538 |
| 456370 | AA234938 | Hs.87384 | ESTs | 2.55 | 4538 |
| 444161 | N52543 | Hs.142940 | ESTs | 2.55 | 3578 |
| 419647 | AA348947 | Hs.91816 | hypothetical protein | 2.55 | 1343 |
| 437231 | BE303000 | Hs.288929 | hypothetical protein FLJ13258 similar to | 2.55 | 3047 |
| 456210 | N49729 | Hs.129819 | ESTs | 2.55 | 4532 |
| 435410 | AL135067 | Hs.117182 | ESTs | 2.55 | 2913 |
| 411136 | AW845266 | | gb: IL2-CT0031-160999-003-B08 CT0031 Homo | 2.55 | 536 |
| 444461 | R53734 | Hs.25978 | ESTs, Weakly similar to 2109260A B cell | 2.55 | 3600 |
| 446035 | NM_006558 | Hs.13565 | Sam68-like phosphotyrosine protein, T-ST | 2.55 | 3720 5663 |
| 426901 | AA827922 | | ESTs | 2.55 | 2109 |
| 437919 | AA772006 | Hs.121630 | ESTs | 2.55 | 3106 |
| 428264 | AA424839 | Hs.98484 | ESTs, Weakly similar to ALU1_HUMAN ALU S | 2.55 | 2234 |
| 413374 | NM_001034 | Hs.75319 | ribonucleotide reductase M2 polypeptide | 2.55 | 4899 713 |
| 431917 | D16181 | Hs.2868 | peripheral myelin protein 2 | 2.55 | 2616 5494 |
| 412115 | AK001763 | Hs.73239 | hypothetical protein FLJ10901 | 2.55 | 4873 607 |
| 439708 | AI761369 | Hs.59584 | hypothetical protein FLJ21144 | 2.55 | 3231 |
| 428782 | X12830 | Hs.193400 | interleukin 6 receptor | 2.55 | 2298 5377 |
| 444091 | AV647924 | Hs.282376 | ESTs | 2.55 | 3574 |
| 434490 | AF143870 | Hs.15246 | ESTs | 2.55 | 2844 |
| 427677 | NM_007045 | Hs.180296 | FGFR1 oncogene partner | 2.55 | 2179 5343 |
| 422296 | AA360231 | Hs.114416 | Homo sapiens, Similar to transducin (bet | 2.55 | 1629 |
| 407788 | BE514982 | Hs.38991 | S100 calcium-binding protein A2 | 2.54 | 195 |
| 430375 | AW371048 | Hs.93758 | H4 histone family, member H | 2.54 | 2477 |
| 456249 | AI206144 | Hs.82508 | HRIHFB2206 protein | 2.54 | 4533 |
| 458981 | AW968318 | Hs.285996 | hypothetical protein FLJ23375 | 2.54 | 4648 |
| 445075 | AI651827 | | ESTs | 2.54 | 3649 |
| 442795 | AW027935 | Hs.251351 | ESTs, Moderately similar to ALU8_HUMAN A | 2.54 | 3471 |
| 451945 | BE504055 | Hs.211420 | ESTs | 2.54 | 4258 |
| 405418 | | | Target Exon | 2.54 | |
| 432878 | BE386490 | Hs.279663 | Pirin | 2.54 | 2707 |
| 434011 | AW953437 | Hs.5486 | clone FLB6214 | 2.53 | 2805 |
| 432942 | AF083955 | Hs.279852 | G protein-coupled receptor | 2.53 | 2714 5519 |
| 403291 | | | Target Exon | 2.53 | |
| 425139 | AW630488 | Hs.25338 | protease, serine, 23 | 2.53 | 1925 |
| 448209 | AW160489 | Hs.20709 | tetraspan 5 | 2.53 | 3951 |
| 434629 | AA789081 | Hs.4029 | glioma-amplified sequence-41 | 2.53 | 2854 |
| 447973 | AB011169 | Hs.20141 | similar to S. cerevisiae SSM4 | 2.53 | 3929 5704 |
| 454294 | AB000734 | Hs.50640 | JAK binding protein | 2.52 | 4476 5795 |
| 446982 | AW500221 | Hs.43616 | gb: UI-HF-BN0-akd-c-03-0-UI.r1 NIH_MGC_50 | 2.52 | 3807 |
| 419735 | AW750056 | Hs.169577 | Homo sapiens cDNA FLJ14743 fis, clone NT | 2.52 | 1354 |
| 431657 | AI345227 | Hs.105448 | ESTs, Weakly similar to B34087 hypotheti | 2.52 | 2580 |
| 447898 | AW969638 | Hs.112318 | 6.2 kd protein | 2.52 | 3921 |
| 457370 | AW970998 | | gb: EST383083 MAGE resequences, MAGK Homo | 2.52 | 4589 |
| 442276 | AI253193 | Hs.129185 | ESTs, Weakly similar to ALU1_HUMAN ALU S | 2.52 | 3410 |
| 439465 | AF086285 | Hs.336620 | Homo sapiens, Similar to RIKEN cDNA 4930 | 2.52 | 3209 |
| 450074 | AI367213 | Hs.14070 | hypothetical protein FLJ14166 | 2.52 | 4103 |
| 424441 | X14850 | Hs.147097 | H2A histone family, member X | 2.52 | 1846 5212 |
| 427295 | AW291212 | Hs.293943 | hypothetical protein MGC11266 | 2.52 | 2139 |
| 424006 | AF054815 | Hs.137548 | CD84 antigen (leukocyte antigen) | 2.52 | 1791 5194 |
| 413326 | H88621 | Hs.19762 | ESTs, Weakly similar to KIAA1140 protein | 2.52 | 711 |
| 443790 | NM_003500 | Hs.9795 | acyl-Coenzyme A oxidase 2, branched chai | 2.52 | 3552 5631 |
| 400995 | | | C11000295*: gi\|12737279\|ref\|XP_012163.1\| | 2.52 | |
| 419767 | W73306 | Hs.306668 | Homo sapiens cDNA FLJ4089 fis, clone MA | 2.51 | 1361 |
| 405357 | | | NM_003371*: Homo sapiens vav 2 oncogene ( | 2.51 | 4734 68 |
| 424720 | M89907 | Hs.152292 | SWI/SNF related, matrix associated, acti | 2.51 | 1877 5228 |
| 408839 | AW277084 | | gb: xp61h09.x1 NCI_CGAP_Ov39 Homo sapiens | 2.51 | 311 |
| 414591 | AI888490 | Hs.55902 | ESTs, Weakly similar to ALU8_HUMAN ALU S | 2.51 | 850 |
| 403055 | | | C2002219*: gi\|12737280\|ref\|XP_006682.2\|k | 2.51 | |
| 437810 | BE246399 | | hypothetical protein | 2.51 | 3093 |
| 413599 | AJ006239 | Hs.75438 | quinoid dihydropteridine reductase | 2.51 | 4904 740 |
| 451259 | NM_006052 | Hs.26146 | Down syndrome critical region gene 3 | 2.51 | 4205 5740 |
| 410817 | AI262789 | Hs.93659 | protein disulfide isomerase related prot | 2.51 | 515 |
| 439943 | AW083789 | Hs.124620 | ESTs | 2.51 | 3247 |
| 409889 | AW630041 | Hs.56937 | suppression of tumorigenicity 14 (colon | 2.51 | 413 |
| 430168 | AW968343 | Hs.145582 | DKFZP434l1735 protein | 2.51 | 2449 |
| 428144 | BE269243 | Hs.182625 | VAMP (vesicle-associated membrane protei | 2.50 | 2222 |
| 402102 | | | Target Exon | 2.50 | |
| 401759 | | | Target Exon | 2.50 | |
| 453899 | AW134536 | Hs.243994 | ESTs | 2.50 | 4447 |
| 405510 | | | ENSP00000233779*: Hypothetical 68.0 kDa p | 2.50 | |
| 442186 | AA984083 | Hs.269746 | ESTs, Weakly similar to T03306 PSD-95/SA | 2.50 | 3405 |
| 423748 | AI149048 | Hs.30211 | hypothetical protein FLJ22313 | 2.50 | 1772 |
| 434378 | AA631739 | Hs.335440 | EST | 2.50 | 2836 |
| 420390 | AA330047 | Hs.191187 | ESTs | 2.50 | 1418 |
| 400272 | | | Eos Control | 2.50 | |
| 427161 | AI024595 | Hs.97508 | a disintegrin and metalloproteinase doma | 2.50 | 2128 |
| 446251 | AW867156 | Hs.282589 | ESTs, Weakly similar to I38022 hypotheti | 2.50 | 3739 |
| 413472 | BE242870 | Hs.75379 | solute carrier family 1 (glial high affi | 2.50 | 724 |
| 410386 | W26187 | Hs.3327 | Homo sapiens cDNA: FLJ22219 fis, clone H | 2.50 | 471 |
| 447698 | AI420156 | Hs.326733 | ESTs | 2.50 | 3891 |
| 456946 | T29678 | Hs.166068 | villin 1 | 2.50 | 4570 |
| 431328 | AA502999 | Hs.291591 | ESTs | 2.50 | 2548 |
| 456583 | AF179897 | Hs.104105 | Meis (mouse) homolog 2 | 2.50 | 4551 5800 |
| 433636 | AA603041 | Hs.296370 | ESTs | 2.50 | 2776 |
| 420325 | AA676544 | Hs.171545 | HIV-1 Rev binding protein | 2.50 | 1413 |
| 422637 | AA399024 | Hs.118836 | myoglobin | 2.50 | 1666 |
| 446586 | N58790 | Hs.268820 | ESTs | 2.50 | 3767 |
| 420520 | AK001978 | Hs.98510 | similar to rab11-binding protein | 2.50 | 1426 5079 |
| 438280 | AW015534 | Hs.217493 | annexin A2 | 2.50 | 3129 |
| 448822 | BE149845 | Hs.289038 | hypothetical protein MGC4126 | 2.50 | 4010 |
| 456497 | AW967956 | Hs.123648 | ESTs, Weakly similar to AF108460 1 ubinu | 2.50 | 4546 |
| 400298 | AA032279 | Hs.61635 | six transmembrane epithelial antigen of | 2.50 | 10 |
| 450277 | AI690071 | Hs.283552 | ESTs, Weakly similar to unnamed protein | 2.50 | 4122 |
| 431719 | AW191608 | Hs.77719 | ESTs, Moderately similar to I38022 hypot | 2.50 | 2589 |
| 409706 | BE158773 | Hs.213207 | ESTs | 2.50 | 397 |
| 420807 | AA280627 | Hs.57846 | ESTs | 2.50 | 1457 |
| 451722 | H86374 | Hs.40861 | ESTs | 2.50 | 4243 |
| 450341 | N90956 | Hs.17230 | hypothetical protein FLJ22087 | 2.50 | 4131 |
| 438552 | AJ245820 | Hs.6314 | type I transmembrane receptor (seizure-r | 2.50 | 3148 5596 |
| 430289 | AK001952 | Hs.238039 | hypothetical protein FLJ11090 | 2.50 | 2461 5440 |
| 416224 | NM_002902 | Hs.79088 | reticulocalbin 2, EF-hand calcium bindin | 2.50 | 4957 983 |
| 418372 | AA311833 | Hs.84318 | replication protein A1 (70 kD) | 2.49 | 1191 |
| 442229 | AI885776 | Hs.8164 | Mulibrey nanism | 2.49 | 3406 |
| 409175 | Y15057 | Hs.50905 | serine/threonine kinase 9 | 2.49 | 344 4815 |
| 420676 | AI434780 | Hs.4248 | vav 2 oncogene | 2.49 | 1443 |
| 433078 | AW015188 | Hs.121575 | Homo sapiens cDNA FLJ12231 fis, clone MA | 2.49 | 2733 |
| 432788 | AA521091 | Hs.178499 | Homo sapiens cDNA: FLJ23117 fis, clone L | 2.49 | 2699 |
| 425811 | AL039104 | Hs.159557 | karyopherin alpha 2 (RAG cohort 1, impor | 2.49 | 1999 |
| 448256 | BE614149 | Hs.20814 | CGI-27 protein | 2.49 | 3952 |
| 402842 | | | ENSP00000241325*: DJ947L8.1.3 (novel CUB | 2.48 | |
| 422799 | AI933199 | Hs.120911 | neurexophilin 4 | 2.48 | 1682 |
| 419517 | AF052107 | Hs.90797 | Homo sapiens clone 23620 mRNA sequence | 2.48 | 1324 |
| 451253 | H48299 | Hs.26126 | claudin 10 | 2.48 | 4204 |
| 453155 | AF052126 | Hs.552 | steroid-5-alpha-reductase, alpha polypep | 2.48 | 4379 5770 |
| 429282 | N27596 | Hs.21342 | ESTs | 2.48 | 2348 |
| 440334 | BE276112 | Hs.7165 | zinc finger protein 259 | 2.48 | 3283 |
| 437852 | BE001836 | Hs.256897 | putative GPCR | 2.47 | 3099 |
| 433339 | AF019226 | Hs.8036 | glioblastoma overexpressed | 2.47 | 2756 |
| 425145 | BE242802 | Hs.154797 | KIAA0090 protein | 2.47 | 1927 |
| 401696 | | | Target Exon | 2.47 | |
| 421877 | AW250380 | Hs.109059 | mitochondrial ribosomal protein L12 | 2.47 | 1575 |
| 427351 | AW402593 | Hs.123253 | hypothetical protein FLJ22009 | 2.47 | 2146 |
| 413835 | AI272727 | Hs.249163 | fatty acid hydroxylase | 2.47 | 756 |
| 407863 | AA317089 | Hs.597 | glutamic-oxaloacetic transaminase 1, sol | 2.47 | 209 |
| 441960 | R84940 | Hs.17118 | hypothetical protein FLJ11730 | 2.47 | 3383 |
| 410099 | AA081630 | | KIAA0036 gene product | 2.47 | 444 |
| 405484 | | | C3002124*: gi\|12737280\|ref\|XP_006682.2\|k | 2.47 | |
| 430105 | X70297 | Hs.2540 | cholinergic receptor, nicotinic, alpha p | 2.46 | 2444 5435 |
| 436823 | AW749865 | Hs.117077 | ESTs, Weakly similar to I38022 hypotheti | 2.46 | 3009 |
| 412754 | AW160375 | Hs.74565 | amyloid beta (A4) precursor-like protein | 2.46 | 666 |
| 412646 | NM_006825 | Hs.74368 | transmembrane protein (63 kD), endoplasmi | 2.46 | 4883 653 |
| 436865 | AW880358 | Hs.339808 | hypothetical protein FLJ10120 | 2.46 | 3014 |
| 451653 | W18193 | | ESTs, Moderately similar to HERC2 [H. sap | 2.46 | 4237 |
| 416097 | BE387371 | Hs.118964 | hypothetical protein FLJ20085 | 2.46 | 971 |
| 448474 | AI792014 | Hs.13809 | hypothetical protein FLJ10648 | 2.46 | 3972 |
| 410686 | AI733735 | Hs.114905 | IRE1, S. cerevisiae, homolog of | 2.45 | 506 |
| 408872 | AI476139 | Hs.13291 | ESTs | 2.45 | 313 |
| 407843 | BE155223 | | gb: PM1-HT0350-070100-006-b01 HT0350 Homo | 2.45 | 206 |
| 413010 | AA393273 | Hs.75133 | transcription factor 6-like 1 (mitochond | 2.45 | 689 |
| 453070 | AK001465 | Hs.31575 | SEC63, endoplasmic reticulum translocon | 2.45 | 4372 |
| 427944 | AA417878 | Hs.48401 | ESTs, Moderately similar to ALU8_HUMAN A | 2.45 | 2205 |
| 428169 | AI928984 | Hs.182793 | golgi phosphoprotein 2 | 2.45 | 2225 |
| 410855 | X97795 | Hs.66718 | RAD54 (S. cerevisiae)-like | 2.45 | 4658 518 |
| 434530 | AU077027 | Hs.3887 | proteasome (prosome, macropain) 26S subu | 2.45 | 2846 |
| 459071 | AI992083 | Hs.346566 | ESTs | 2.45 | 4654 |
| 425516 | BE000707 | Hs.29567 | ESTs | 2.45 | 1972 |
| 413230 | R72391 | Hs.16512 | hypothetical protein FLJ21079 | 2.45 | 702 |
| 405010 | | | Target Exon | 2.45 | |
| 438578 | AA811244 | | ESTs | 2.45 | 3151 |
| 405704 | | | NM_001844*: Homo sapiens collagen, type I | 2.45 | 4739 73 |
| 408629 | N58905 | Hs.119206 | ESTs | 2.45 | 285 |
| 415069 | AA159831 | Hs.29286 | ESTs, Weakly similar to I49636 DNA-bindi | 2.45 | 905 |
| 437748 | AF234882 | Hs.5814 | suppression of tumorigenicity 7 | 2.45 | 3087 5591 |
| 411402 | BE297855 | Hs.69855 | NRAS-related gene | 2.45 | 552 |
| 406671 | AA129547 | Hs.285754 | met proto-oncogene (hepatocyte growth fa | 2.45 | 82 |
| 407321 | AA156538 | Hs.22546 | gb: zo34c06.s1 Stratagene colon (937204) | 2.45 | 152 |
| 408418 | AW963897 | Hs.44743 | KIAA1435 protein | 2.45 | 266 |
| 443230 | T80789 | Hs.132973 | ESTs | 2.45 | 3511 |
| 442329 | AA991795 | Hs.129289 | ESTs | 2.45 | 3420 |
| 446782 | AI653048 | Hs.144006 | solute carrier family 6 (neurotransmitte | 2.45 | 3785 |
| 410062 | AW974756 | Hs.46476 | ESTs | 2.45 | 437 |
| 425170 | AU077315 | Hs.154970 | transcription factor CP2 | 2.45 | 1932 |
| 445571 | AI378000 | Hs.158489 | ESTs, Weakly similar to Z184_HUMAN ZINC | 2.45 | 3686 |
| 450684 | AA872605 | Hs.25333 | interleukin 1 receptor, type II | 2.45 | 4148 |
| 420608 | BE548277 | Hs.103104 | ESTs | 2.45 | 1436 |
| 418882 | NM_004996 | Hs.89433 | ATP-binding cassette, sub-family C (CFTR | 2.45 | 1254 5033 |
| 424081 | NM_006413 | Hs.139120 | ribonuclease P (30 kD) | 2.45 | 1801 5197 |
| 431242 | AA987742 | Hs.347534 | KIAA1201 protein | 2.45 | 2543 |
| 439492 | AF086310 | Hs.103159 | ESTs | 2.45 | 3212 |
| 434360 | AW015415 | Hs.127780 | ESTs | 2.45 | 2833 |
| 420747 | BE294407 | Hs.99910 | phosphofructokinase, platelet | 2.45 | 1448 |
| 403167 | | | Target Exon | 2.45 | |
| 429227 | AI961456 | Hs.21275 | hypothetical protein FLJ11011 | 2.45 | 2339 |
| 435800 | AI248285 | Hs.118348 | ESTs | 2.44 | 2945 |
| 409935 | AW511413 | Hs.278025 | ESTs | 2.44 | 420 |
| 407712 | X66893 | Hs.37953 | Fanconi anemia, complementation group C | 2.44 | 185 4780 |
| 449112 | BE564123 | Hs.23060 | DKFZP564F0522 protein | 2.44 | 4038 |
| 420460 | AA262331 | Hs.48376 | Homo sapiens clone HB-2 mRNA sequence | 2.44 | 1423 |
| 431044 | U93850 | Hs.259855 | elongation factor-2 kinase | 2.44 | 2525 5468 |
| 416817 | AA398045 | Hs.104679 | ESTs | 2.44 | 1028 |
| 429604 | AK001851 | Hs.210778 | hypothetical protein FLJ10989 | 2.44 | 2393 5413 |
| 410717 | AW817608 | Hs.130849 | peptide deformylase-like protein | 2.44 | 510 |
| 428028 | U52112 | Hs.182018 | interleukin-1 receptor-associated kinase | 2.44 | 2215 |
| 424572 | M19650 | Hs.179600 | 2′,3′-cyclic nucleotide 3′ phosphodieste | 2.44 | 1859 5218 |
| 448100 | AV655272 | Hs.20252 | novel Ras family protein | 2.44 | 3941 |
| 435468 | AW362803 | Hs.166271 | ESTs | 2.44 | 2916 |
| 448112 | AW245919 | Hs.34969 | hypothetical protein DKFZp566N034 | 2.43 | 3944 |
| 428242 | H55709 | Hs.2250 | leukemia inhibitory factor (cholinergic | 2.43 | 2233 |
| 400247 | | | Eos Control | 2.43 | |
| 439955 | AW203959 | Hs.149532 | ESTs | 2.43 | 3249 |
| 424135 | AW994455 | Hs.140978 | Homo sapiens mRNA: cDNA DKFZp762H106 (fr | 2.43 | 1807 |
| 417534 | NM_004998 | Hs.82251 | myosin IE | 2.43 | 1100 4989 |
| 405276 | | | NM_019854: Homo sapiens HMT1 (hnRNP methy | 2.43 | 4733 67 |
| 403349 | | | ephrin-B3 | 2.43 | |
| 443281 | AI042304 | Hs.74635 | dihydrolipoamide dehydrogenase (E3 compo | 2.43 | 3516 |
| 411358 | R47479 | Hs.94761 | KIAA1691 protein | 2.43 | 548 |
| 423493 | AI815965 | Hs.129683 | ubiquitin-conjugating enzyme E2D 1 (Homo | 2.43 | 1753 |
| 432586 | AA568548 | | ESTs | 2.43 | 2681 |
| 423393 | R37772 | Hs.21420 | p21-activated protein kinase 6 | 2.42 | 1741 |
| 407907 | AI752235 | Hs.41270 | procollagen-lysine, 2-oxoglutarate 5-dio | 2.42 | 214 |
| 418699 | BE539639 | Hs.173030 | ESTs, Weakly similar to ALU8_HUMAN ALU S | 2.42 | 1229 |
| 432828 | AB042326 | Hs.287402 | chondroitin 4-sulfotransferase | 2.42 | 2702 5515 |
| 447388 | AW630534 | Hs.76277 | Homo sapiens, clone MGC: 9381, mRNA, comp | 2.42 | 3854 |
| 432623 | AA557351 | Hs.152448 | ESTs, Moderately similar to S14147 multi | 2.42 | 2686 |
| 442452 | AI479592 | Hs.324342 | ESTs | 2.42 | 3432 |
| 446777 | AW138243 | | ESTs | 2.42 | 3784 |
| 405451 | | | Homo sapiens glutaminyl-peptide cyclotra | 2.42 | |
| 428166 | AA423849 | Hs.79530 | M5-14 protein | 2.41 | 2224 |
| 419705 | AW368634 | Hs.154331 | ESTs | 2.41 | 1351 |
| 403961 | | | Target Exon | 2.41 | |
| 422243 | AW803733 | Hs.23585 | hypothetical protein MGC12250 | 2.41 | 1618 |
| 441941 | AI953261 | Hs.169813 | hypothetical protein FLJ23040 | 2.41 | 3380 |
| 457332 | AA961694 | Hs.105187 | kinesin protein 9 gene | 2.41 | 4587 |
| 424511 | BE300512 | Hs.193557 | ESTs, Moderately similar to ALU7_HUMAN A | 2.41 | 1854 |
| 431563 | AI027643 | Hs.120912 | ESTs | 2.41 | 2567 |
| 447501 | AI954692 | Hs.159400 | ESTs | 2.41 | 3871 |
| 418650 | BE386750 | Hs.86978 | prolyl endopeptidase | 2.41 | 1219 |
| 415558 | AA885143 | Hs.125719 | ESTs | 2.41 | 937 |
| 450791 | R84948 | Hs.60473 | RFamide-related peptide precursor | 2.41 | 4166 |
| 427673 | AI989709 | | gb: ws35f01.x1 NCI_CGAP_GC6 Homo sapiens | 2.41 | 2176 |
| 437576 | BE514383 | | prothymosin, alpha (gene sequence 28) | 2.40 | 3070 |
| 417831 | H16423 | Hs.82685 | CD47 antigen (Rh-related antigen, integr | 2.40 | 1128 |
| 421778 | AA428000 | Hs.283072 | actin related protein 2/3 complex, subun | 2.40 | 1563 |
| 449082 | BE387561 | Hs.22981 | DKFZP586M1523 protein | 2.40 | 4034 |
| 410219 | T98226 | Hs.171952 | occludin | 2.40 | 457 |
| 431583 | AL042613 | Hs.262476 | S-adenosylmethionine decarboxylase 1 | 2.40 | 2571 |
| 446173 | BE565849 | Hs.14158 | copine III | 2.40 | 3733 |
| 409929 | R38772 | Hs.172619 | myelin transcription factor 1-like | 2.40 | 419 |
| 405863 | | | Target Exon | 2.40 | |
| 417587 | AI911492 | Hs.12489 | ESTs | 2.40 | 1104 |
| 434159 | AW135214 | Hs.191828 | ESTs | 2.40 | 2816 |
| 448964 | N63996 | Hs.289083 | ESTs | 2.40 | 4022 |
| 425216 | U81504 | Hs.155172 | adaptor-related protein complex 3, beta | 2.40 | 1938 5252 |
| 430448 | AI633553 | Hs.13303 | Homo sapiens cDNA: FLJ21784 fis, clone H | 2.40 | 2485 |
| 414754 | AW938668 | | gb: PM1-DT0063-160200-003-c07 DT0063 Homo | 2.40 | 874 |
| 443073 | AI032321 | | ESTs | 2.40 | 3494 |
| 450904 | R07118 | Hs.189924 | ESTs | 2.40 | 4174 |
| 425908 | BE281602 | Hs.161751 | zinc finger protein 256 | 2.40 | 2011 |
| 412846 | AW961245 | Hs.55896 | Homo sapiens PAC clone RP5-978E18 from 7 | 2.40 | 677 |
| 415641 | Z44481 | | gb: HSC21E071 normalized infant brain cDN | 2.40 | 940 |
| 405258 | | | Target Exon | 2.40 | |
| 445866 | H20899 | Hs.13399 | Homo sapiens clone 25032 mRNA sequence | 2.40 | 3704 |
| 402412 | | | Target Exon | 2.40 | |
| 447842 | AW160804 | Hs.247302 | twisted gastrulation | 2.40 | 3913 |
| 434405 | AF171055 | Hs.20030 | Homo sapiens thioredoxin reductase 3 (TR | 2.40 | 2838 5547 |
| 428328 | AA426080 | Hs.292812 | ESTs, Weakly similar to I38022 hypotheti | 2.40 | 2240 |
| 400567 | | | Target Exon | 2.40 | |
| 453374 | AI990091 | Hs.75893 | ankyrin 3, node of Ranvier (ankyrin G) | 2.40 | 4401 |
| 429250 | H56585 | Hs.198308 | tryptophan rich basic protein | 2.40 | 2343 |
| 426996 | AW968934 | Hs.173108 | Homo sapiens cDNA: FLJ21897 fis, clone H | 2.40 | 2119 |
| 442607 | AA507576 | Hs.288361 | Homo sapiens cDNA: FLJ22696 fis, clone H | 2.40 | 3452 |
| 440534 | BE561168 | | gb: 601344888F1 NIH_MGC_8 Homo sapiens cD | 2.40 | 3295 |
| 420968 | AW968775 | | ESTs | 2.40 | 1470 |
| 443829 | AI087954 | Hs.23348 | S-phase kinase-associated protein 2 (p45 | 2.40 | 3557 |
| 417581 | R26968 | Hs.24104 | ESTs, Weakly similar to ALU7_HUMAN ALU S | 2.40 | 1103 |
| 442072 | AI740832 | Hs.12311 | Homo sapiens clone 23570 mRNA sequence | 2.40 | 3395 |
| 443197 | Z43613 | | gb: HSC1GD091 normalized infant brain cDN | 2.40 | 3507 |
| 412193 | AI684467 | Hs.144057 | ESTs | 2.40 | 617 |
| 408826 | AF216077 | Hs.48376 | Homo sapiens clone HB-2 mRNA sequence | 2.40 | 309 |
| 422321 | AA906427 | Hs.181035 | hypothetical protein MGC11296 | 2.40 | 1631 |
| 447769 | AW873704 | Hs.320831 | Homo sapiens cDNA FLJ14597 fis, clone NT | 2.40 | 3903 |
| 422631 | BE218919 | Hs.118793 | hypothetical protein FLJ10688 | 2.40 | 1665 |
| 447958 | AW796524 | Hs.68644 | Homo sapiens microsomal signal peptidase | 2.40 | 3926 |
| 423430 | AF112481 | Hs.128501 | RAD54, S. cerevisiae, homolog of, B | 2.40 | 1744 |
| 422981 | AF026445 | Hs.122752 | TATA box binding protein (TBP)-associate | 2.40 | 1706 5165 |
| 418330 | BE409405 | | ESTs | 2.40 | 1187 |
| 434627 | AI221894 | Hs.39311 | ESTs | 2.40 | 2853 |
| 445098 | AL050272 | Hs.12305 | DKFZP566B183 protein | 2.40 | 3651 5651 |
| 446711 | AF169692 | Hs.12450 | protocadherin 9 | 2.40 | 3776 5675 |
| 447495 | AW401864 | Hs.18720 | programmed cell death 8 (apoptosis-induc | 2.40 | 3868 |
| 450825 | AC005954 | Hs.25527 | tight junction protein 3 (zona occludens | 2.40 | 4170 5736 |
| 421526 | AL080121 | Hs.105460 | DKFZP564O0823 protein | 2.40 | 1534 5114 |
| 431605 | AW972407 | Hs.124370 | gb: EST384498 MAGE resequences, MAGL Homo | 2.39 | 2574 |
| 429612 | AF062649 | Hs.252587 | pituitary tumor-transforming 1 | 2.39 | 2396 5416 |
| 419170 | BE002798 | Hs.287850 | integral membrane protein 1 | 2.39 | 1285 |
| 437271 | AL137445 | Hs.28846 | Homo sapiens mRNA; cDNA DKFZp566O134 (fr | 2.39 | 3052 |
| 458097 | AW341135 | Hs.58104 | ESTs | 2.39 | 4610 |
| 426105 | AW962732 | | gb: EST374805 MAGE resequences, MAGG Homo | 2.39 | 2027 |
| 414075 | U11862 | Hs.75741 | amiloride binding protein 1 (amine oxida | 2.39 | 4913 785 |
| 445994 | NM_004724 | Hs.13512 | ZW10 (Drosophila) homolog, centromere/ki | 2.39 | 3716 5661 |
| 420186 | NM_015925 | Hs.95697 | liver-specific bHLH-Zip transcription fa | 2.39 | 1392 5066 |
| 404109 | | | C6000844*: gi\|7497891\|pir\|\|T20194 hypothe | 2.39 | 52 |
| 431341 | AA307211 | Hs.251531 | proteasome (prosome, macropain) subunit, | 2.39 | 2549 |
| 409703 | NM_006187 | Hs.56009 | 2′-5′-oligoadenylate synthetase 3 (100 k | 2.39 | 396 4831 |
| 445834 | AI913290 | Hs.145532 | ESTs, Weakly similar to FV1 MOUSE FRIEND | 2.38 | 3701 |
| 449636 | AI656608 | Hs.281328 | ESTs, Weakly similar to T00378 KIAA0641 | 2.38 | 4071 |
| 425081 | X74794 | Hs.154443 | minichromosome maintenance deficient (S. | 2.38 | 1918 5246 |
| 414368 | W70171 | Hs.75939 | uridine monophosphate kinase | 2.38 | 818 |
| 431698 | AI492369 | | ESTs | 2.38 | 2585 |
| 431549 | AA507036 | Hs.170673 | ESTs | 2.38 | 2565 |
| 418731 | AI264688 | Hs.1197 | heat shock 10 kD protein 1 (chaperonin 10 | 2.38 | 1233 |
| 414111 | BE047679 | Hs.152982 | hypothetical protein FLJ13117 | 2.38 | 789 |
| 431356 | AW499632 | Hs.288512 | Homo sapiens cDNA FLJ11632 fis, clone HE | 2.38 | 2550 |
| 443048 | AW013982 | Hs.250114 | ESTs | 2.38 | 3490 |
| 431049 | AA846576 | Hs.103267 | hypothetical protein FLJ22548 similar to | 2.38 | 2527 |
| 409129 | AW296699 | Hs.103521 | serine arginine-rich pre-mRNA splicing f | 2.38 | 337 |
| 453323 | AF034102 | Hs.32951 | solute carrier family 29 (nucleoside tra | 2.37 | 4395 5775 |
| 449022 | NM_016331 | Hs.22879 | zinc finger protein ANC_2H01 | 2.37 | 4029 5718 |
| 418068 | AW971155 | Hs.293902 | ESTs, Weakly similar to ISHUSS protein d | 2.37 | 1157 |
| 431317 | AA502682 | | gb: ng23d01.s1 NCI_CGAP_Ov2 Homo sapiens | 2.37 | 2547 |
| 430315 | NM_004293 | Hs.239147 | guanine deaminase | 2.37 | 2469 5444 |
| 443162 | T49951 | Hs.9029 | DKFZP434G032 protein | 2.37 | 3500 |
| 418583 | AA604379 | Hs.86211 | hypothetical protein | 2.37 | 1214 |
| 417008 | AA191708 | Hs.325825 | Homo sapiens cDNA FLJ20848 fis, clone AD | 2.37 | 1050 |
| 459057 | AW005512 | | ESTs, Weakly similar to A61262 collagen | 2.37 | 4653 |
| 447120 | AI380413 | Hs.233297 | ESTs | 2.37 | 3824 |
| 427712 | AI368024 | Hs.283696 | ESTs | 2.37 | 2187 |
| 435099 | AC004770 | Hs.4756 | flap structure-specific endonuclease 1 | 2.37 | 2896 5563 |
| 421878 | AA299652 | Hs.111496 | Homo sapiens cDNA FLJ11643 fis, clone HE | 2.37 | 1576 |
| 434614 | AI249502 | Hs.29669 | ESTs | 2.37 | 2852 |
| 413437 | BE313164 | Hs.75361 | gene from NF2/meningioma region of 22q12 | 2.37 | 722 |
| 428821 | H91282 | Hs.286232 | Homo sapiens cDNA: FLJ23190 fis, clone L | 2.36 | 2304 |
| 419897 | X90826 | Hs.93649 | upstream transcription factor 2, c-fos i | 2.36 | 1366 5059 |
| 437016 | AU076916 | Hs.5398 | guanise mosphosphate synthetase | 2.36 | 3027 |
| 421011 | BE300408 | Hs.119699 | hypothetical protein FLJ12969 | 2.36 | 1474 |
| 409407 | AW967370 | Hs.342655 | Homo sapiens cDNA FLJ13289 fis, clone OV | 2.36 | 367 |
| 415204 | T27434 | | gb: hbc2294 Human pancreatic islet Homo s | 2.36 | 915 |
| 449162 | AI632740 | Hs.10476 | ESTs | 2.36 | 4041 |
| 429238 | NM_002849 | Hs.198288 | protein tyrosine phosphalase, receptor t | 2.36 | 2341 5395 |
| 449955 | AI676010 | Hs.224043 | ESTs | 2.36 | 4093 |
| 449791 | AI248740 | Hs.133323 | ESTs | 2.36 | 4083 |
| 422515 | AW500470 | Hs.117950 | multifunctional polypeptide similar to S | 2.36 | 1651 |
| 419102 | AA234098 | Hs.42424 | ESTs, Weakly similar to 2004399A chromos | 2.36 | 1276 |
| 411734 | AW374954 | Hs.71779 | Homo sapiens DNA from chromosome 19, cos | 2.36 | 572 |
| 405723 | | | Target Exon | 2.36 | |
| 456623 | AI084125 | Hs.108106 | transcription factor | 2.36 | 4555 |
| 442013 | AA506476 | Hs.82689 | Human DNA sequence from clone RP11-353C1 | 2.36 | 3388 |
| 429770 | AI766047 | Hs.99736 | ESTs | 2.36 | 2415 |
| 407317 | AI204033 | Hs.30792 | ESTs, Weakly similar to I38022 hypotheti | 2.35 | 151 |
| 421910 | NM_014586 | Hs.109437 | hormonally upregulated neu tumor-associa | 2.35 | 1579 5128 |
| 404120 | | | C5000537*: gi\|3298595\|gb\|AAC41376.1\|(AF0 | 2.35 | |
| 452483 | AI903731 | Hs.106357 | valosin-containing protein | 2.35 | 4316 |
| 435755 | AA805263 | Hs.123176 | ESTs | 2.35 | 2940 |
| 428550 | AW297880 | Hs.98661 | ESTs | 2.35 | 2269 |
| 449158 | AI990849 | | ESTs | 2.35 | 4040 |
| 449256 | AA059050 | Hs.59847 | ESTs | 2.35 | 4051 |
| 442617 | AW340093 | Hs.130538 | ESTs | 2.35 | 3454 |
| 459557 | N58315 | | gh: yv68g06.s1 Soares fetal liver spleen | 2.35 | 4672 |
| 425363 | BE161577 | Hs.10119 | hypothetical protein FLJ14957 | 2.35 | 1954 |
| 448030 | N30714 | Hs.325960 | membrane-spanning 4-domains, subfamily A | 2.35 | 3936 |
| 437159 | AL050072 | Hs.306313 | Homo sapiens mRNA; cDNA DKFZp566E1346 (f | 2.35 | 3040 |
| 440462 | T71629 | Hs.100554 | ESTs | 2.35 | 3290 |
| 458375 | AI885827 | Hs.7149 | ESTs, Weakly similar to T13963 formin re | 2.35 | 4621 |
| 454645 | AW811928 | | gb: RC2-ST0168-240300-017-b02 ST0168 Homo | 2.35 | 4493 |
| 435126 | AI393666 | Hs.42315 | p10-binding protein | 2.35 | 2898 |
| 411226 | AW833022 | | gb: RC3-TT0005-191099-012-d04 TT0005 Homo | 2.35 | 540 |
| 402285 | | | sclerostin | 2.35 | |
| 423805 | AA331242 | Hs.197030 | gb: EST35320 Embryo, 8 week I Homo sapien | 2.35 | 1777 |
| 446338 | AI289121 | Hs.206978 | ESTs | 2.35 | 3745 |
| 400817 | | | Target Exon | 2.35 | |
| 459507 | AW298279 | Hs.255756 | ESTs | 2.35 | 4669 |
| 425368 | AB014595 | Hs.155976 | cullin 4B | 2.35 | 1956 5258 |
| 425212 | AW962253 | Hs.171618 | ESTs | 2.35 | 1937 |
| 426098 | NM_014906 | Hs.166351 | KIAA1072 protein | 2.35 | 2026 5291 |
| 457653 | AI820719 | Hs.76853 | DnaJ (Hsp40) homolog, subfamily A, membe | 2.35 | 4597 |
| 432281 | AK001239 | Hs.274263 | hypothetical protein FLJ10377 | 2.35 | 2652 5505 |
| 418592 | X99226 | Hs.284153 | Fanconi anemia, complementation group A | 2.35 | 1215 5020 |
| 402505 | | | C1003830: gi\|3983382\|gb\|AAD13319.1\| (AF10 | 2.35 | |
| 410415 | BE241626 | Hs.285833 | Homo sapiens cDNA: FLJ22135 fis, clone H | 2.35 | 475 |
| 418216 | AA662240 | Hs.283099 | AF15q14 protein | 2.35 | 1171 |
| 459200 | Y09306 | Hs.30148 | homeodomain-isteracting protein kinase 3 | 2.35 | 4658 5818 |
| 413320 | NM_005191 | Hs.838 | CD80 antigen (CD28 antigen ligand 1, B7- | 2.35 | 4898 710 |
| 430084 | AA748878 | Hs.281764 | ESTs | 2.35 | 2443 |
| 412043 | BE156622 | Hs.333371 | Homo sapiens clone TA40 untranslated mRN | 2.35 | 604 |
| 446651 | AA393907 | Hs.97179 | ESTs | 2.35 | 3775 |
| 431696 | AA259068 | Hs.267819 | protein phosphatase 1, regulatory (inhib | 2.35 | 2584 |
| 451065 | AW295132 | Hs.222231 | ESTs, Weakly similar to granule cell mar | 2.35 | 4188 |
| 451232 | AI769922 | Hs.20023 | ESTs | 2.35 | 4201 |
| 416018 | AW138239 | Hs.78977 | proprotein convertase subtilisin/kexin t | 2.35 | 966 |
| 448762 | H19006 | Hs.184780 | ESTs | 2.35 | 4004 |
| 432226 | AW182766 | Hs.273558 | phosphate cytidylyltransferase 1, cholin | 2.35 | 2645 |
| 435062 | BE501995 | Hs.282228 | ESTs | 2.35 | 2892 |
| 417239 | AA854504 | Hs.85956 | ESTs, Weakly similar to B28096 line-1 pr | 2.35 | 1074 |
| 450724 | R55428 | | gb: yj79b05.r1 Soares breast 2NbHBst Homo | 2.35 | 4154 |
| 425048 | H05468 | Hs.164502 | ESTs | 2.35 | 1914 |
| 453455 | AA063553 | Hs.221931 | ESTs, Weakly similar to JC1087 RNA helic | 2.35 | 4408 |
| 434874 | N62448 | Hs.293970 | methylmalonate-semialdehyde dehydrogenas | 2.35 | 2876 |
| 409187 | AF154830 | Hs.50966 | carbamoyl-phosphate synthetase 1, mitoch | 2.35 | 347 4816 |
| 444261 | AA298958 | Hs.10724 | MDS023 protein | 2.35 | 3585 |
| 414205 | BE382438 | | Homo sapiens, clone IMAGE: 3690478, mRNA, | 2.35 | 799 |
| 428862 | NM_000346 | Hs.2316 | SRY (sex determining region Y)-box 9(Ca | 2.35 | 2313 5382 |
| 448633 | AA311426 | Hs.21635 | tubulin, gamma 1 | 2.35 | 3990 |
| 418758 | AW9593151 | Hs.172012 | hypothetical protein DKFZp434J037 | 2.35 | 1237 |
| 420783 | AI659838 | Hs.99923 | lectin, galactoside-binding, soluble, 7 | 2.35 | 1453 |
| 439866 | AA280717 | Hs.6727 | Ras-GTPase activating protein SH3 domain | 2.35 | 3240 |
| 454003 | AA058944 | Hs.116602 | Homo sapiens, clone IMAGE: 4154008, mRNA, | 2.34 | 4460 |
| 458866 | BE616694 | Hs.288042 | hypothetical protein FLJ14299 | 2.34 | 4641 |
| 425532 | AB007915 | Hs.158286 | KIAA0446 gene product | 2.34 | 1976 5271 |
| 436420 | AA443966 | Hs.31595 | ESTs | 2.34 | 2984 |
| 420542 | NM_000505 | Hs.1321 | coagulation factor XII (Hageman factor) | 2.34 | 1428 5080 |
| 417389 | BE260964 | Hs.82045 | midkine (neurite growth-promoting factor | 2.34 | 1091 |
| 407704 | BE315072 | Hs.78768 | malignant cell expression-enhanced gene/ | 2.34 | 184 |
| 421649 | AA721217 | Hs.106415 | peroxisome proliferative activated recep | 2.34 | 1549 |
| 424042 | Y10601 | Hs.137674 | ankyrin-like with transmembrane domains | 2.34 | 1794 5195 |
| 414883 | AA926960 | | CDC28 protein kinase 1 | 2.34 | 887 |
| 414270 | L20852 | Hs.347527 | solute carrier family 20 (phosphate tran | 2.34 | 4915 806 |
| 428698 | AA852773 | Hs.334838 | KIAA1866 protein | 2.34 | 2283 |
| 410170 | AI700481 | Hs.299629 | peroxisomal long-chain acyl-coA thioeste | 2.34 | 452 |
| 422567 | AF111178 | Hs.118407 | glypican 6 | 2.34 | 1658 5154 |
| 418418 | R61527 | Hs.237517 | ESTs | 2.34 | 1198 |
| 407661 | AW063497 | Hs.279821 | ESTs | 2.33 | 178 |
| 450088 | AW292933 | Hs.254110 | ESTs | 2.33 | 4104 |
| 427279 | AW971391 | Hs.28113 | ESTs, Weakly similar to KIAA0349 [H. sapi | 2.33 | 2136 |
| 417133 | N49848 | Hs.104091 | ESTs | 2.33 | 1064 |
| 419457 | AA243146 | Hs.209334 | ESTs, Moderately similar to S23A_HUMAN P | 2.33 | 1315 |
| 405215 | | | Target Exon | 2.33 | |
| 449186 | AW291876 | Hs.196986 | ESTs | 2.33 | 4042 |
| 445929 | AI089660 | Hs.323401 | dpy-30-like protein | 2.33 | 3714 |
| 425265 | BE245297 | | gb: TCBAP1E2482 Pediatric pre-B cell acut | 2.33 | 1946 |
| 402008 | | | Target Exon | 2.33 | |
| 449207 | AL044222 | Hs.23255 | nucleoporin 155 kD | 2.33 | 4045 |
| 432842 | AW674093 | Hs.334822 | hypothetical protein MGC4485 | 2.33 | 2705 |
| 420089 | AW003785 | Hs.170267 | ESTs | 2.33 | 1382 |
| 447966 | AA340605 | Hs.105887 | ESTs, Weakly similar to Homolog of rat Z | 2.33 | 3928 |
| 418951 | F07809 | Hs.89506 | paired box gene 6 (aniridia, keratitis) | 2.33 | 1262 |
| 405387 | | | NM_022170*: Homo sapiens Williams-Beuren | 2.33 | 4736 70 |
| 412124 | H43378 | | Homo sapiens cDNA: FLJ23156 fis, clone L | 2.33 | 611 |
| 417018 | M16038 | Hs.80887 | v-yes-1 Yamaguchi sarcoma viral related | 2.33 | 1053 4976 |
| 405573 | | | Target Exon | 2.32 | |
| 422253 | W81526 | Hs.118329 | ESTs, Moderately similar to GAD_HUMAN GA | 2.32 | 1621 |
| 422363 | T55979 | Hs.115474 | replication factor C (activator 1) 3 (38 | 2.32 | 1636 |
| 413952 | AA806187 | Hs.289101 | glucose regulated protein, 58 kD | 2.32 | 769 |
| 407649 | BE066724 | Hs.37427 | erythrocyte membrane protein band 4.1 (e | 2.32 | 177 |
| 413492 | D87470 | Hs.75400 | KIAA0280 protein | 2.32 | 4903 728 |
| 413753 | U17760 | Hs.75517 | laminin, beta 3 (nicein (125 kD), kalinin | 2.32 | 4906 750 |
| 446506 | AI123118 | Hs.15159 | chemokine-like factor, alternatively spl | 2.32 | 3757 |
| 444734 | NM_001360 | Hs.11806 | 7-dehydrocholesterol reductase | 2.32 | 3622 5644 |
| 437210 | AA311443 | Hs.293563 | Homo sapiens mRNA; cDNA DKFZp586E2317 (f | 2.32 | 3045 |
| 407027 | U63312 | | gb: Human cosmid LL12NCO1-242E1, ETV6 gen | 2.31 | 120 4763 |
| 407252 | AA659037 | Hs.163780 | ESTs | 2.31 | 146 |
| 408075 | AA382881 | Hs.42409 | CGI-146 protein | 2.31 | 231 |
| 452767 | AW014195 | Hs.61472 | ESTs, Weakly similar to YAE6_YEAST HYPOT | 2.31 | 4343 |
| 426516 | BE262660 | Hs.170197 | glutamic-oxaloacetic transaminase 2, mit | 2.31 | 2074 |
| 423864 | BE275607 | Hs.1708 | chaperonin containing TCP1, subunit 3 (g | 2.31 | 1781 |
| 402663 | | | C1002133: gi\|12697931\|dbj\|BAB21784.1\|(AB | 2.31 | |
| 452482 | BE155356 | | gb: PM1-HT0350-160300-009-d06 HT0350 Homo | 2.31 | 4315 |
| 419535 | AW139550 | Hs.115173 | ESTs | 2.31 | 1326 |
| 446335 | BE174202 | Hs.156067 | Homo sapiens cDNA: FLJ21061 fis, clone C | 2.31 | 3744 |
| 420552 | AK000492 | Hs.98806 | hypothetical protein | 2.31 | 1430 5081 |
| 457461 | AL122087 | Hs.272304 | Homo sapiens mRNA; cDNA DKFZp564C0371 (f | 2.31 | 4591 |
| 401917 | | | RAN binding protein 3 | 2.31 | |
| 427678 | BE267756 | Hs.180312 | mitochondrial ribosomal protein S16 | 2.31 | 2180 |
| 429857 | AF089897 | Hs.294030 | topoisomerase-related function protein 4 | 2.30 | 2419 5428 |
| 437613 | R19892 | Hs.10267 | MIL1 protein | 2.30 | 3075 |
| 429162 | AK001250 | Hs.197642 | hypothetical protein FLJ10388 | 2.30 | 2334 5391 |
| 427051 | BE178110 | Hs.173374 | Homo sapiens cDNA FLJ10500 fis, clone NT | 2.30 | 2123 |
| 432407 | AA221036 | Hs.13273 | gb: zr03f12.r1 Stratagene NT2 neuronal pr | 2.30 | 2664 |
| 415099 | AI492170 | Hs.77917 | ubiquitin carboxyl-terminal esterase L3 | 2.30 | 907 |
| 432543 | AA552690 | Hs.152423 | Homo sapiens cDNA: FLJ21274 fis, clone C | 2.30 | 2679 |
| 446237 | AW270515 | Hs.149596 | Homo sapiens, Similar to RIKEN cDNA 2310 | 2.30 | 3738 |
| 436668 | AA831857 | Hs.209071 | ESTs | 2.30 | 3001 |
| 452146 | AW135654 | | p47 | 2.30 | 4276 |
| 404494 | | | C8001441*V: gi\|8923061\|ref\|NP_060114.1\|hy | 2.30 | |
| 426262 | AI792141 | Hs.196270 | folate transporter/carrier | 2.30 | 2045 |
| 439019 | AF085902 | Hs.271737 | ESTs | 2.30 | 3182 |
| 400639 | | | C10000999*: gi\|2143593\|pir\|\|S55277 annexi | 2.30 | 19 |
| 454204 | AW818498 | | gb: QVO-ST0236-171299-075-b02 ST0236 Homo | 2.30 | 4472 |
| 424194 | BE245833 | Hs.169854 | gb: TCBAP1E1908 Pediatric pre-B cell acut | 2.30 | 1813 |
| 421093 | AA906434 | Hs.3776 | zinc finger protein 216 | 2.30 | 1480 |
| 435276 | AI022399 | Hs.186934 | ESTs | 2.30 | 2906 |
| 451084 | N30113 | Hs.299014 | ESTs | 2.30 | 4190 |
| 409078 | AW327515 | | ESTs | 2.30 | 331 |
| 412661 | N32860 | Hs.24611 | ESTs, Weakly similar to I54374 gene NF2 | 2.30 | 657 |
| 418150 | AA973762 | Hs.22222 | hypothetical protein MGC10854 | 2.30 | 1164 |
| 411688 | AW953440 | | gb: EST365510 MAGE resequences, MAGB Homo | 2.30 | 570 |
| 437863 | AA769989 | | ESTs, Moderately similar to ZN91_HUMAN Z | 2.30 | 3100 |
| 445523 | Z30118 | Hs.293788 | ESTs, Moderately similar to unnamed prot | 2.30 | 3682 |
| 421674 | T10707 | Hs.296355 | hypothetical protein FLJ23138 | 2.30 | 1553 |
| 405705 | | | C12000104*: gi\|4503519\|ref\|NP_003745.1\| e | 2.30 | |
| 409872 | AW502313 | | gb: UI-HF-BR0p-ajs-d-08-0-UI.r1 NIH_MGC_5 | 2.30 | 411 |
| 440612 | BE561384 | | gb: 601344969F1 NIH_MGC_8 Homo sapiens CD | 2.30 | 3305 |
| 422408 | AI340322 | Hs.156928 | Homo sapiens cDNA: FLJ21942 fis, clone H | 2.30 | 1641 |
| 429433 | AA452899 | Hs.213586 | ESTs, Weakly similar to KIAA1353 protein | 2.30 | 2368 |
| 406930 | U04691 | | gb: Human olfactory receptor (OR17-219) g | 2.30 | 111 4757 |
| 448472 | BE612556 | Hs.47783 | B aggressive lymphoma gene | 2.30 | 3971 |
| 419852 | AW503756 | Hs.286184 | hypothetical protein dJ551D2.5 | 2.30 | 1363 |
| 440097 | AI079949 | Hs.134192 | ESTs | 2.30 | 3261 |
| 455540 | BE080231 | | gb: RC4-BT0629-120200-012-f11 BT0629 Homo | 2.30 | 4514 |
| 424316 | AA676403 | Hs.145078 | regulator of differentiation (in S. pomb | 2.30 | 1829 |
| 404423 | | | C8000067*: gi\|10432400\|emb\|CAC10290.1\| (A | 2.30 | |
| 427548 | AA813784 | Hs.123001 | ESTs | 2.30 | 2164 |
| 438171 | AW976507 | | ESTs | 2.30 | 3123 |
| 431611 | U58766 | Hs.264428 | tissue specific transplantation antigen | 2.30 | 2575 5482 |
| 440100 | BE382685 | Hs.158549 | ESTs, Weakly similar to T2D3_HUMAN TRANS | 2.30 | 3263 |
| 417750 | AI267720 | Hs.260523 | synovial sarcoma, translocated to X chro | 2.30 | 1120 |
| 426088 | AF038007 | Hs.166196 | ATPase, Class I, type 8B, member 1 | 2.30 | 2024 5289 |
| 414485 | W27026 | Hs.182625 | VAMP (vesicle-associated membrane protei | 2.30 | 835 |
| 411007 | AA311529 | Hs.67619 | hypothetical protein My014 | 2.30 | 527 |
| 410512 | AA085603 | Hs.250570 | hypothetical protein MGC3180 | 2.30 | 487 |
| 426427 | M86699 | Hs.169840 | UK protein kinase | 2.30 | 2065 5303 |
| 437897 | AA770561 | Hs.146170 | hypothetical protein FLJ22969 | 2.30 | 3104 |
| 431462 | AW583672 | Hs.256311 | granin-like neuroendocrine peptide precu | 2.30 | 2557 |
| 458820 | BE552151 | Hs.108118 | hypothetical protein FLJ22474 | 2.30 | 4640 |
| 422678 | AA247778 | Hs.119155 | Homo sapiens mRNA full length insert cDN | 2.30 | 1672 |
| 430640 | AA482636 | Hs.209561 | KIAA1715 protein | 2.30 | 2502 |
| 428157 | AI738719 | Hs.198427 | hexokinase 2 | 2.30 | 2223 |
| 443015 | R33261 | Hs.6614 | ESTs, Weakly similar to A43932 mucin 2 p | 2.30 | 3487 |
| 426860 | U04953 | Hs.172801 | isoleucine-tRNA synthetase | 2.29 | 2107 5325 |
| 437150 | R51407 | Hs.77910 | 3-hydroxy-3-methylglutaryl-Coenzyme A sy | 2.29 | 3038 |
| 401913 | | | ENSP00000249158*: CDNA | 2.29 | |
| 442592 | BE566623 | Hs.29899 | ESTs, Weakly similar to G02075 transcrip | 2.29 | 3450 |
| 446038 | AW162917 | Hs.321635 | gb: au90d03.y1 Schneider fetal brain 0000 | 2.29 | 3721 |
| 416820 | NM_000402 | Hs.80206 | glucose-6-phosphate dehydrogenase | 2.29 | 1029 4969 |
| 421535 | AB002359 | Hs.105478 | phosphoribosylformylglycinamidine syntha | 2.29 | 1535 5115 |
| 446567 | NM_007247 | Hs.15384 | AP1 gamma subunit binding protein 1 | 2.29 | 3764 5672 |
| 451134 | AA318315 | Hs.25999 | hypothetical protein FLJ22195 | 2.29 | 4196 |
| 441659 | BE564162 | Hs.250820 | hypothebcal protein FLJ14827 | 2.29 | 3366 |
| 434182 | W20309 | Hs.118520 | G-protein gamma-12 subunit | 2.29 | 2818 |
| 447118 | AB014599 | Hs.330988 | Homo sapiens, Similar to Bicaudal D (Dro | 2.29 | 3823 5682 |
| 443044 | N28522 | Hs.8935 | quinolinate phosphoribosyltransferase (n | 2.29 | 3489 |
| 405028 | | | C9001589: gi\|548751\|sp\|P36241\|RL19_DROME | 2.29 | |
| 418747 | AJ249977 | Hs.88049 | protein kinase, AMP-activated, gamma 3 n | 2.29 | 1235 5028 |
| 402189 | | | ENSP00000247423*: D.siglec precursor | 2.29 | |
| 408743 | AL110246 | Hs.47367 | KIAA1785 protein | 2.29 | 301 |
| 408697 | AW419069 | Hs.209670 | ESTs | 2.29 | 294 |
| 408249 | AW271838 | Hs.44038 | pellino (Drosophila) homolog 2 | 2.29 | 249 |
| 432279 | N95104 | Hs.274260 | ATP-binding cassette, sub-family C (CFTR | 2.28 | 2651 |
| 446229 | AI744964 | Hs.14449 | KIAA1609 protein | 2.28 | 3736 |
| 421828 | AW891965 | | histone deacetylase 3 | 2.28 | 1569 |
| 451351 | AW058261 | Hs.321435 | ESTs, Weakly similar to ALU1_HUMAN ALU S | 2.28 | 4214 |
| 412445 | X51362 | Hs.73893 | dopamine receptor D2 | 2.28 | 4879 632 |
| 425671 | AF193612 | Hs.159142 | lunatic fringe (Drosophila) homolog | 2.28 | 1984 5274 |
| 425360 | BE547704 | Hs.15276 | gb: 601076309F1 NIH_MGC_12 Homo sapiens c | 2.28 | 1953 |
| 454560 | AW807281 | | gb: MR4-ST0062-240300-003-g01 ST0062 Homo | 2.28 | 4490 |
| 430016 | NM_004736 | Hs.227656 | xenotropic and polytropic retrovirus rec | 2.28 | 2437 5432 |
| 457234 | AW968360 | Hs.14355 | Homo sapiens cDNA FLJ13207 fis, clone NT | 2.28 | 4584 |
| 458132 | AW247012 | Hs.103267 | hypothetical protein FLJ22548 similar to | 2.28 | 4612 |
| 432871 | NM_016142 | Hs.279617 | steroid dehydrogenase homolog | 2.28 | 2706 5517 |
| 409119 | AA531133 | Hs.4253 | hypothetical protein MGC2574 | 2.27 | 335 |
| 452700 | AI859390 | Hs.288940 | five-span transmembrane protein M83 | 2.27 | 4336 |
| 400414 | AF083118 | Hs.283968 | Homo sapiens CATX-2 mRNA, complete cds | 2.27 | 16 4688 |
| 401804 | | | C17000304*: gi\|9801259\|emb\|CAC03558.1\| (A | 2.27 | |
| 428599 | AB033078 | Hs.186613 | sphingosine-1-phosphate lyase 1 | 2.27 | 2276 5367 |
| 409465 | AW393810 | Hs.78054 | gb: QV4-TT0008-251099-016-e11 TT0008 Homo | 2.27 | 376 |
| 434067 | H18913 | Hs.124023 | Homo sapiens cDNA FLJ14218 fis, clone NT | 2.27 | 2812 |
| 447656 | NM_003726 | Hs.19126 | src kinase-associated phosphoprotein of | 2.27 | 3887 5695 |
| 448641 | R31845 | Hs.21666 | insulin-like 4 (placenta) | 2.27 | 3991 |
| 439442 | AA310162 | Hs.169248 | cytochrome c | 2.27 | 372 |
| 409213 | U61412 | Hs.51133 | PTK6 protein tyrosine kinase 6 | 2.27 | 352 4818 |
| 411942 | AW877015 | | gb: QV2-PT0010-250300-096-f12 PT0010 Homo | 2.27 | 588 |
| 415279 | F04237 | Hs.1447 | glial fibrillary acidic protein | 2.27 | 923 |
| 448667 | Z78394 | Hs.4896 | Homo sapiens cDNA: FLJ22046 fis, clone H | 2.27 | 3995 |
| 439714 | AW367812 | Hs.199961 | ESTs, Weakly similar to ALU7_HUMAN ALU S | 2.27 | 399 |
| 444984 | H15474 | Hs.132898 | fatty acid desaturase 1 | 2.27 | 3643 |
| 411737 | AW160339 | Hs.71791 | hypothetical protein | 2.27 | 573 |
| 428054 | AI948688 | Hs.266619 | ESTs | 2.26 | 2218 |
| 407844 | AW073716 | Hs.8037 | ESTs | 2.26 | 207 |
| 447484 | AA464839 | Hs.292566 | hypothetical protein FLJ14697 | 2.26 | 3866 |
| 442723 | AI804331 | Hs.99423 | ATP-dependent RNA helicase | 2.26 | 3463 |
| 433626 | AF078859 | Hs.86347 | hypothetical protein | 2.26 | 2773 5537 |
| 425960 | AW410646 | Hs.164649 | hypothetical protein DKFZp434H247 | 2.26 | 2014 |
| 456258 | AW976410 | Hs.289069 | Homo sapiens clone FLB3411 PRO0852 mRNA, | 2.26 | 4535 |
| 408482 | NM_000676 | Hs.45743 | adenosine A2b receptor | 2.26 | 270 4798 |
| 427752 | AA470687 | Hs.104772 | ESTs | 2.26 | 2194 |
| 444572 | R50145 | Hs.270549 | HZFw1 protein | 2.26 | 3610 |
| 422010 | AA302049 | Hs.31181 | Homo sapiens cDNA: FLJ23230 fis, clone C | 2.26 | 1593 |
| 433698 | H24201 | Hs.247423 | adducin 2 (beta) | 2.26 | 2781 |
| 416636 | N32536 | Hs.42645 | solute carrier family 16 (monocarboxylic | 2.26 | 1008 |
| 422248 | AL109695 | Hs.113657 | Homo sapiens mRNA full length insert cDN | 2.26 | 1620 |
| 431604 | AF175265 | Hs.264190 | vacuolar protein sorting 35 (yeast homol | 2.26 | 2573 5481 |
| 447211 | AL161961 | Hs.17767 | KIAA1554 protein | 2.25 | 3830 |
| 414906 | AA157911 | Hs.72200 | ESTs | 2.25 | 890 |
| 450746 | D82673 | Hs.278589 | general transcription factor II, i | 2.25 | 4158 |
| 412326 | R07566 | Hs.73817 | small inducible cytokine A3 (homologous | 2.25 | 624 |
| 422955 | AW967824 | Hs.324237 | ESTs | 2.25 | 1697 |
| 448918 | AB011152 | Hs.22572 | KIAA0580 protein | 2.25 | 4019 5716 |
| 402229 | | | mitochondrial ribosomal protein S2 | 2.25 | |
| 446550 | AW500453 | Hs.34455 | gb: UI-HF-BN0-akb-c-11-0-UI.r1 NIH_MGC_50 | 2.25 | 3760 |
| 425702 | N59555 | | gb: yv76f05.s1 Soares fetal liver spleen | 2.25 | 1991 |
| 416707 | H78163 | Hs.14005 | ESTs | 2.25 | 1015 |
| 450754 | AW204237 | | ESTs, Weakly similar to ALU1_HUMAN ALU S | 2.25 | 4162 |
| 428898 | AB033070 | Hs.194408 | KIAA1244 protein | 2.25 | 2316 5383 |
| 404877 | | | NM_005365: Homo sapiens melanoma antigen, | 2.25 | 4729 63 |
| 441926 | AI015051 | Hs.130953 | ESTs | 2.25 | 3379 |
| 424003 | BE274717 | Hs.137506 | Homo sapiens, clone IMAGE: 3605104, mRNA, | 2.25 | 1789 |
| 411782 | H30518 | | gb: yp41b03.s1 Soares retina N2b5HR Homo | 2.25 | 578 |
| 443741 | AW451759 | Hs.145420 | ESTs | 2.25 | 3547 |
| 404341 | | | Target Exon | 2.25 | |
| 438632 | AI910521 | Hs.123493 | ESTs | 2.25 | 3155 |
| 442481 | N99828 | | gb: za32c04.r1 Soares fetal liver spleen | 2.25 | 3434 |
| 401160 | | | Target Exon | 2.25 | |
| 413940 | AI633205 | Hs.159914 | ESTs, Weakly similar to I78885 serine/th | 2.25 | 766 |
| 401925 | | | sialyltransferase 1 (beta-galactoside al | 2.25 | |
| 410401 | AW673335 | Hs.259641 | ESTs | 2.25 | 473 |
| 418764 | N30531 | Hs.42215 | protein phosphatase 1, regulatory subuni | 2.25 | 1238 |
| 407687 | AK002011 | Hs.37558 | hypothetical protein FLJ11149 | 2.25 | 180 4778 |
| 449065 | AI627393 | Hs.258998 | ESTs, Weakly similar to high mobility gr | 2.25 | 4032 |
| 452243 | AL355715 | Hs.28555 | programmed cell death 9 (PDCD9) | 2.25 | 4287 5756 |
| 417124 | BE122762 | Hs.25338 | ESTs | 2.25 | 1063 |
| 432821 | BE170702 | Hs.279005 | solute carrier family 21 (organic anion | 2.25 | 2701 |
| 425416 | AK000909 | Hs.157103 | hypothetical protein FLJ12644 | 2.25 | 1960 |
| 446182 | AV656995 | Hs.269904 | ESTs | 2.25 | 3734 |
| 428180 | AI129767 | Hs.182874 | guanine nucleotide binding protein (G pr | 2.25 | 2226 |
| 422285 | AI803103 | | gb: tc14e06.x1 Soares_NhHMPu_S1 Homo sapi | 2.25 | 1627 |
| 444326 | AI939357 | | ESTs | 2.25 | 3589 |
| 446147 | AL133064 | Hs.14051 | Homo sapiens mRNA; cDNA DKFZp434A2417 (f | 2.25 | 3727 5665 |
| 432808 | NM_015985 | Hs.278973 | angiopoietin-3 | 2.25 | 2700 5514 |
| 434639 | R83159 | Hs.33366 | EST | 2.25 | 2858 |
| 442430 | R89164 | | double ring-finger protein, Dorfin | 2.25 | 3428 |
| 406374 | | | C16001364: gi\|11067373\|ref\|NP_067689.1\| C | 2.25 | |
| 428405 | Y00762 | Hs.2266 | cholinergic receptor, nicotinic, alpha p | 2.25 | 2250 5357 |
| 428466 | AF151063 | Hs.184456 | hypothetical protein | 2.25 | 2261 5360 |
| 417046 | AA192639 | | gb: zq01h09.r1 Stratagene muscle 937209 H | 2.25 | 1057 |
| 439239 | AI031540 | Hs.235331 | ESTs | 2.25 | 3194 |
| 431820 | AW410408 | Hs.271167 | L-pipecolic acid oxidase | 2.25 | 2602 |
| 413273 | U75679 | Hs.75257 | stem-loop (histone) binding protein | 2.25 | 4896 704 |
| 400234 | | | NM_005336: Homo sapiens high density lipo | 2.25 | 4 4683 |
| 413691 | AB023173 | Hs.75478 | ATPase, Class VI, type 11B | 2.25 | 4905 748 |
| 436856 | AI469355 | Hs.127310 | ESTs | 2.25 | 3011 |
| 424685 | W21223 | Hs.151734 | nuclear transport factor 2 (placental pr | 2.25 | 1872 |
| 435438 | H84421 | Hs.4890 | ubiquitin-conjugating enzyme E2E 3 (Homo | 2.25 | 2915 |
| 440043 | BE277457 | Hs.30661 | hypothetical protein MGC4606 | 2.25 | 3256 |
| 453575 | AB023211 | Hs.33455 | peptidyl arginine deiminase, type II | 2.24 | 4419 5780 |
| 424689 | D43947 | Hs.151761 | KIAA0100 gene product | 2.24 | 1874 5226 |
| 428206 | AB020643 | Hs.183006 | KIAA0836 protein | 2.24 | 2229 5351 |
| 417207 | N92226 | Hs.338218 | ESTs | 2.24 | 1072 |
| 430979 | AI479755 | Hs.129010 | ESTs | 2.24 | 2523 |
| 402530 | | | Target Exon | 2.24 | |
| 427691 | AW194426 | Hs.20726 | UDP-N-acetyl-alpha-D-galactosamine: polyp | 2.24 | 2181 |
| 434206 | AW136973 | | ESTs, Weakly similar to S69890 mitogen i | 2.24 | 2821 |
| 452017 | AF109302 | Hs.27495 | prostate cancer associated protein 7 | 2.24 | 4263 |
| 422127 | AW504286 | Hs.112049 | SET binding factor 1 | 2.24 | 1606 |
| 408949 | AF189011 | Hs.49163 | putative ribonuclease III | 2.24 | 319 4809 |
| 441085 | AW136551 | Hs.181245 | Homo sapiens cDNA FLJ12532 fis, clone NT | 2.24 | 3331 |
| 425140 | AB014567 | Hs.154740 | TBP-interacting protein | 2.24 | 1926 5247 |
| 423840 | AA332434 | Hs.72465 | ESTs, Weakly similar to non-lens beta ga | 2.24 | 1779 |
| 420428 | AA262050 | Hs.156148 | hypothetical protein FLJ13231 | 2.24 | 1420 |
| 448497 | BE613269 | Hs.21893 | hypothetical protein DKFZp761N0624 | 2.24 | 3977 |
| 411400 | AA311919 | Hs.69851 | nucleolar protein family A, member 1 (H/ | 2.23 | 551 |
| 431683 | AK001749 | Hs.267604 | hypothetical protein FLJ10450 | 2.23 | 2583 5486 |
| 421222 | AA306049 | Hs.102669 | DKFZP434O125 protein | 2.23 | 1491 |
| 425118 | AU076611 | Hs.154672 | methylene tetrahydrofolate dehydrogenase | 2.23 | 1923 |
| 447948 | AI620923 | Hs.46679 | hypothetical protein FLJ20739 | 2.23 | 3924 |
| 403332 | | | Target Exon | 2.23 | |
| 431123 | X77723 | Hs.326056 | rabaptin-5 | 2.23 | 2531 |
| 421247 | BE391727 | Hs.102910 | general transcription factor IIH, polype | 2.23 | 1495 |
| 421568 | W85858 | Hs.99804 | ESTs | 2.23 | 1539 |
| 458376 | AB023179 | Hs.9059 | KIAA0962 protein | 2.23 | 4622 5816 |
| 452388 | BE019696 | Hs.29287 | retinoblastoma-binding protein 8 | 2.23 | 4302 |
| 401952 | | | Target Exon | 2.23 | |
| 402819 | | | ENSP00000235150: DJ1174N9.1 (novel protei | 2.23 | |
| 437582 | AW673321 | Hs.46903 | hypothetical protein FLJ12838 | 2.23 | 3071 |
| 439180 | AI393742 | Hs.199067 | v-erb-b2 avian erythroblastic leukemia v | 2.22 | 3190 |
| 409960 | BE261944 | | hexokinase 1 | 2.22 | 422 |
| 451617 | C01056 | | hypothetical protein FLJ12577 | 2.22 | 4233 |
| 435956 | AF269255 | Hs.22604 | lysosomal apyrase-like protein 1 | 2.22 | 2954 5573 |
| 431863 | AA188185 | Hs.289043 | spindlin | 2.22 | 2609 |
| 409456 | U34962 | Hs.54473 | cardiac-specific homeo box | 2.22 | 374 4825 |
| 425354 | U62027 | Hs.155935 | complement component 3a receptor 1 | 2.22 | 1952 5257 |
| 445664 | AW968638 | Hs.237691 | ESTs, Weakly similar to KIAA0601 protein | 2.22 | 3692 |
| 453915 | AA588721 | Hs.286218 | ribosomal protein L44 | 2.22 | 4451 |
| 431896 | AW297844 | Hs.101428 | ESTs | 2.22 | 2613 |
| 429377 | AA813192 | Hs.200596 | KIAA0547 gene product | 2.22 | 2362 |
| 409189 | AA125984 | | gb: zn27h06.r1 Stratagene neuroepithelium | 2.22 | 348 |
| 452488 | N74921 | Hs.184389 | ESTs | 2.22 | 4318 |
| 445356 | AW304777 | Hs.249690 | ESTs | 2.22 | 3672 |
| 402014 | | | Target Exon | 2.22 | |
| 420014 | AI248571 | Hs.13913 | KIAA1577 protein | 2.22 | 1373 |
| 455618 | BE155563 | | gb: PM4-HT0352-171199-001-C05 HT0352 Homo | 2.21 | 4516 |
| 437063 | AA351109 | | Tax1 (human T-cell leukemia virus type I | 2.21 | 3033 |
| 419508 | AW997938 | Hs.90786 | ATP-binding cassette, sub-family C (CFTR | 2.21 | 1323 |
| 409185 | AW961601 | Hs.252406 | hypothetical protein FLJ12296 similar to | 2.21 | 346 |
| 456999 | AA319798 | Hs.298581 | eukaryotic translation elongation factor | 2.21 | 4573 |
| 400203 | | | Eos Control | 2.21 | |
| 412490 | AW803564 | Hs.288850 | Homo sapiens cDNA: FLJ22528 fis, clone H | 2.21 | 637 |
| 434878 | AW975086 | | gb: EST387192 MAGE resequences, MAGN Homo | 2.21 | 2878 |
| 429898 | AW117322 | Hs.42366 | ESTs | 2.21 | 2423 |
| 444784 | D12485 | Hs.11951 | ectonucleotide pyrophosphatase/phosphodi | 2.21 | 3629 5646 |
| 447197 | R36075 | | gb: yh88b01.s1 Soares placenta Nb2HP Homo | 2.21 | 3828 |
| 417691 | AU076610 | Hs.82399 | low density lipoprotein receptor defect | 2.21 | 1112 |
| 449949 | AI675753 | Hs.186530 | ESTs, Weakly similar to S65657 alpha-1C- | 2.21 | 4092 |
| 410553 | AW016824 | Hs.272068 | hypothetical protein MGC14128 | 2.21 | 491 |
| 421551 | AF063539 | Hs.97300 | ESTs | 2.21 | 1537 |
| 414600 | NM_005647 | Hs.76536 | transducin (beta)-like 1 | 2.21 | 4921 852 |
| 428726 | AA432195 | Hs.98694 | ESTs | 2.20 | 2287 |
| 452753 | AA028049 | Hs.277728 | SEC14 (S. cerevisiae)-like 2 | 2.20 | 4342 |
| 434916 | AF161383 | Hs.284207 | Homo sapiens, Similar to RIKEN cDNA 1110 | 2.20 | 2879 5558 |
| 408113 | T82427 | Hs.194101 | Homo sapiens cDNA: FLJ20869 fis, clone A | 2.20 | 235 |
| 428385 | AF112213 | Hs.184062 | putative Rab5-interacting protein | 2.20 | 2247 5356 |
| 459376 | BE258770 | | Homo sapiens, clone IMAGE: 3344506, mRNA, | 2.20 | 4667 |
| 455475 | AW948126 | | gb: RC0-MT0013-280300-031-a12 MT0013 Homo | 2.20 | 4510 |
| 403549 | | | ENSP00000187471: HSNFRK, | 2.20 | |
| 434356 | AW467161 | Hs.78563 | ubiquitin-conjugating enzyme E2G 1 (Homo | 2.20 | 2832 |
| 445292 | AV653264 | Hs.13982 | Homo sapiens cDNA FLJ14666 fis, clone NT | 2.20 | 3666 |
| 403481 | | | Target Exon | 2.20 | |
| 410101 | AI338045 | Hs.203559 | hypothetical protein FLJ12701 | 2.20 | 445 |
| 401438 | | | ENSP00000238580*: SIMILARITY IS TO THE EN | 2.20 | |
| 421642 | AF172066 | Hs.106346 | retinoic acid repressible protein | 2.20 | 1548 5118 |
| 403429 | | | C3000329*: gi\|8922921\|ref\|NP_060821.1\| hy | 2.20 | |
| 440129 | AA865818 | | ESTs, Weakly similar to S71886 Ste20-lik | 2.20 | 3266 |
| 414571 | BE410746 | Hs.22868 | protein tyrosine phosphatase, non-recept | 2.20 | 846 |
| 451859 | H44491 | Hs.252938 | ESTs, Weakly similar to ALU1_HUMAN ALU S | 2.20 | 4253 |
| 410859 | AW807604 | | gb: MR4-ST0098-120100-001-b02 ST0098 Homo | 2.20 | 519 |
| 403154 | | | NM_022780*: Homo sapiens hypothetical pro | 2.20 | 43 4712 |
| 447054 | AW086454 | Hs.169248 | ESTs | 2.20 | 3815 |
| 443108 | W86975 | | ESTs | 2.20 | 3497 |
| 450058 | AW452752 | Hs.256034 | ESTs | 2.20 | 4101 |
| 410434 | AF051152 | Hs.63668 | toll-like receptor 2 | 2.20 | 478 4847 |
| 411436 | AW846433 | | gb: QV0-CT0179-070300-143-b02 CT0179 Homo | 2.20 | 556 |
| 414898 | AA157726 | Hs.264330 | N-acylsphingosine amidohydrolase (acid c | 2.20 | 889 |
| 448501 | AA332316 | | hypothetical protein FLJ13159 | 2.20 | 3978 |
| 457378 | AW972118 | Hs.100002 | HSPC162 protein | 2.20 | 4590 |
| 422890 | Z43784 | | ankyrin 3, node of Ranvier (ankyrin G) | 2.20 | 1690 |
| 438814 | AA826278 | | ESTs | 2.20 | 3167 |
| 425810 | AI923627 | Hs.31903 | ESTs | 2.20 | 1998 |
| 436683 | AW991278 | Hs.57787 | ESTs | 2.20 | 3003 |
| 410465 | AW963279 | Hs.123373 | ESTs, Weakly similar to ALU1_HUMAN ALU S | 2.20 | 481 |
| 433597 | AA708205 | Hs.100343 | ESTs | 2.20 | 2771 |
| 437838 | AI307229 | | ESTs | 2.20 | 3097 |
| 403858 | | | Target Exon | 2.20 | |
| 445325 | AF052115 | Hs.12514 | Homo sapiens clone 23688 mRNA sequence | 2.20 | 3668 |
| 450750 | H83398 | Hs.40489 | ESTs | 2.20 | 4161 |
| 417173 | U61397 | Hs.81424 | ubiquitin-like 1 (sentrin) | 2.20 | 1068 4979 |
| 437587 | AI591222 | Hs.72325 | Human DNA sequence from clone RP1-187J11 | 2.20 | 3072 |
| 423020 | AA383092 | Hs.1608 | replication protein A3 (14 kD) | 2.20 | 1711 |
| 429904 | AL080077 | Hs.225997 | Homo sapiens mRNA; cDNA DKFZp564C0962 (f | 2.20 | 2426 |
| 421387 | AF059566 | Hs.103983 | solute carrier family 5 (sodium iodide s | 2.20 | 1516 5105 |
| 429626 | U36787 | Hs.211571 | holocytochrome c synthase (cytochrome c | 2.20 | 2399 5418 |
| 411911 | AW875951 | | gb: CM1-PT0013-131299-067-f09 PT0013 Homo | 2.20 | 586 |
| 438093 | BE206885 | Hs.6076 | COP9 (constitutive photomorphogenic, Ara | 2.20 | 3117 |
| 432262 | AW197269 | Hs.127240 | hypothetical protein FLJ10211 | 2.20 | 2649 |
| 414178 | AW957372 | Hs.46791 | ESTs, Weakly similar to I38022 hypotheti | 2.19 | 797 |
| 424766 | BE388855 | Hs.152978 | proteaseome (prosome, macropain) 28 subu | 2.19 | 1884 |
| 455299 | AW891612 | | gb: CM3-NT0089-040500-174-a04 NT0089 Homo | 2.19 | 4506 |
| 422416 | BE019557 | Hs.11900 | hypothetical protein FLJ14972 | 2.19 | 1642 |
| 428758 | AA433988 | Hs.98502 | CA125 antigen; mucin 16 | 2.19 | 2292 |
| 410600 | AW575742 | | ESTs, Moderately similar to S65657 alpha | 2.19 | 496 |
| 400540 | | | Target Exon | 2.19 | |
| 445176 | AI878907 | Hs.12379 | ELAV (embryonic lethal, abnormal vision, | 2.19 | 3659 |
| 445350 | AF052112 | Hs.12540 | lysophospholipase I | 2.19 | 3671 |
| 420718 | NM_002301 | Hs.99881 | lactate dehydrogenase C | 2.19 | 1445 5084 |
| 436605 | AI187742 | Hs.125562 | ESTs | 2.19 | 2996 |
| 421353 | AW292857 | Hs.255130 | ESTs | 2.19 | 1511 |
| 435080 | AI831760 | Hs.155111 | hypothetical protein FLJ14428 | 2.19 | 2893 |
| 419252 | AW138434 | Hs.129805 | ESTs | 2.19 | 1293 |
| 419591 | AF090900 | Hs.91393 | Homo sapiens cDNA: FLJ21887 fis, clone H | 2.19 | 1338 5054 |
| 410687 | U24389 | Hs.65436 | lysyl oxidase-like 1 | 2.19 | 4853 507 |
| 446849 | AU076617 | Hs.16251 | cleavage and polyadenylation specific fa | 2.19 | 3795 |
| 414740 | R61532 | Hs.87016 | hypothetical protein FLJ22938 | 2.19 | 871 |
| 432707 | AW604865 | Hs.158515 | hypothetical protein MGC13038 | 2.19 | 2692 |
| 448362 | AA641767 | Hs.21015 | hypothetical protein DKFZp564L0864 simil | 2.19 | 3960 |
| 430637 | BE160081 | Hs.256290 | S100 calcium-binding protein A11 (calgiz | 2.19 | 2501 |
| 432348 | AA534353 | Hs.194081 | ESTs, Weakly similar to I38022 hypotheti | 2.19 | 2657 |
| 419444 | NM_002496 | Hs.90443 | Target CAT | 2.19 | 1314 5048 |
| 401403 | | | Target Exon | 2.19 | |
| 432621 | AI298501 | Hs.21192 | ESTs, Weakly similar to T46428 hypotheti | 2.19 | 2685 |
| 428281 | AA194554 | Hs.183434 | ATPase, H transporting, lysosomal (vacuo | 2.18 | 2236 |
| 421525 | AW977559 | Hs.4193 | DKFZP586O1624 protein | 2.18 | 1533 |
| 433139 | AB029826 | Hs.47649 | methylcrotonoyl-Coenzyme A carboxylase 1 | 2.18 | 2738 5529 |
| 444298 | Z17870 | | gb: HSDHII020 Stratagene cDNA library Hum | 2.18 | 3586 |
| 428074 | BE387770 | Hs.182378 | colony stimulating factor 2 receptor, al | 2.18 | 2219 |
| 406423 | | | C19000229*: gi\|6753826\|ref\|NP_034311.1\|f | 2.18 | |
| 442911 | AI023895 | Hs.190587 | ESTs | 2.18 | 3479 |
| 425524 | AB007866 | Hs.158249 | KIAA0406 gene product | 2.18 | 1974 5269 |
| 413063 | AL035737 | Hs.75184 | chitinase 3-like 1 (cartilage glycoprote | 2.18 | 692 |
| 450998 | BE387614 | Hs.25797 | splicing factor 3b, subunit 4, 49 kD | 2.18 | 4181 |
| 440035 | BE561589 | | hypothetical protein FLJ21839 | 2.18 | 3254 |
| 451025 | AW028689 | Hs.301985 | ESTs | 2.18 | 4182 |
| 449008 | AW578003 | Hs.22826 | tropomodulin 3 (ubiquitous) | 2.18 | 4026 |
| 426174 | AA547959 | Hs.115838 | Homo sapiens similar to Echinoidin (LOC1 | 2.18 | 2036 |
| 416322 | BE019494 | Hs.79217 | pyrroline-5-carboxylate reductase 1 | 2.17 | 986 |
| 401711 | | | C16000267: gi\|6330617\|dbj\|BAA86537.1\|(AB | 2.17 | |
| 446989 | AK001898 | Hs.16740 | hypothetical protein FJ11036 | 2.17 | 3808 5679 |
| 416375 | H95567 | Hs.124700 | ESTs | 2.17 | 991 |
| 453985 | N44545 | Hs.251865 | ESTs | 2.17 | 4457 |
| 402942 | | | Target Exon | 2.17 | |
| 400120 | | | Eos Control | 2.17 | |
| 401138 | | | Target Exon | 2.17 | |
| 414108 | AI267592 | Hs.75761 | SFRS protein kinase 1 | 2.17 | 788 |
| 427715 | BE245274 | Hs.180428 | KIAA1181 protein | 2.17 | 2188 |
| 448407 | AI498957 | Hs.170861 | ESTs, Weakly similar to Z195_HUMAN ZINC | 2.17 | 3965 |
| 448745 | H51548 | Hs.21899 | solute carrier family 35 (UDP-galactose | 2.17 | 4001 |
| 436265 | AA731331 | Hs.190668 | ESTs | 2.17 | 2972 |
| 406237 | | | Target Exon | 2.17 | |
| 402322 | | | Target Exon | 2.17 | |
| 416812 | H91010 | Hs.44940 | ESTs | 2.17 | 1025 |
| 429462 | AI890356 | | Homo sapiens, clone IMAGE: 3536432, mRNA, | 2.17 | 2373 |
| 438098 | AI076370 | Hs.134037 | ESTs | 2.17 | 3118 |
| 442134 | BE552359 | Hs.225092 | ESTs | 2.17 | 3400 |
| 445922 | AI337316 | Hs.147998 | ESTs | 2.17 | 3713 |
| 419590 | AF005043 | Hs.91390 | poly (ADP-ribose) glycohydrolase | 2.17 | 1337 5053 |
| 446290 | AW769683 | Hs.6734 | ESTs, Weakly similar to S26650 DNA-bindi | 2.17 | 3742 |
| 413313 | NM_002047 | Hs.283108 | glycyl-tRNA synthetase | 2.17 | 4897 709 |
| 404661 | | | C9000306*: gi\|12737280\|ref\|XP_006682.2\|k | 2.17 | |
| 448854 | AW245617 | Hs.77703 | hypothetical protein FLJ11506 | 2.16 | 4014 |
| 414169 | AA136169 | Hs.149335 | ESTs | 2.16 | 796 |
| 414161 | AA136106 | Hs.184852 | KIAA1553 protein | 2.16 | 794 |
| 445070 | NM_000677 | Hs.258 | adenosine A3 receptor | 2.16 | 3648 5650 |
| 446830 | BE179030 | | Human DNA sequence from clone RP5-1174N9 | 2.16 | 3793 |
| 414636 | AL120259 | Hs.76691 | stannin | 2.16 | 856 |
| 432626 | AA471098 | Hs.278544 | acetyl-Coenzyme A acetyltransferase 2 (a | 2.16 | 2687 |
| 428582 | BE336699 | Hs.185055 | BENE protein | 2.16 | 2272 |
| 453710 | AL119136 | Hs.236131 | homeodomain-interacting protein kinase 2 | 2.16 | 4425 |
| 438510 | AL080220 | Hs.6285 | DKFZP586P0123 protein | 2.16 | 3143 5595 |
| 454034 | NM_000691 | Hs.575 | aldehyde dehydrogenase 3 family, member | 2.16 | 4462 5792 |
| 451991 | AA768353 | Hs.163709 | ESTs, Moderately similar to I38022 hypot | 2.16 | 4261 |
| 415282 | R44308 | Hs.242302 | ESTs | 2.16 | 924 |
| 415443 | T07353 | Hs.7948 | ESTs | 2.16 | 931 |
| 409267 | NM_012453 | Hs.52515 | transducin (beta)-like 2 | 2.16 | 357 4821 |
| 440300 | N39760 | Hs.8859 | Homo sapiens, Similar to RIKEN cDNA 5830 | 2.16 | 3279 |
| 440494 | BE618768 | Hs.7232 | acetyl-Coenzyme A carboxylase alpha | 2.16 | 3293 |
| 435550 | AI224456 | Hs.324507 | H. sapiens polyA site DNA | 2.16 | 2927 |
| 415914 | AA306033 | Hs.78915 | GA-binding protein transcription factor, | 2.16 | 960 |
| 436700 | AI693690 | Hs.301406 | hypothetical protein PP3501 | 2.15 | 3005 |
| 454150 | AA131893 | Hs.154088 | hypothetical protein FLJ22756 | 2.15 | 4470 |
| 423540 | AI038192 | Hs.95361 | EGF-like repeats and discoidin I-like do | 2.15 | 1755 |
| 400084 | | | Eos Control | 2.15 | |
| 453129 | R35265 | Hs.237946 | ESTs, Weakly similar to TRHY_HUMAN TRICH | 2.15 | 4377 |
| 421861 | S78798 | Hs.108966 | phosphatidylinositol-4-phosphate 5-kinas | 2.15 | 1572 5126 |
| 421506 | BE302796 | Hs.105097 | thymidine kinase 1, soluble | 2.15 | 1528 |
| 405673 | | | NM_022775: Homo sapiens hypothetical prot | 2.15 | 4738 72 |
| 413774 | AA131782 | Hs.182314 | ESTs | 2.15 | 753 |
| 452177 | AI863447 | Hs.268180 | gb: tz48f01.x1 NCI_CGAP_Bm52 Homo sapien | 2.15 | 4279 |
| 450166 | AA429504 | Hs.8765 | ESTs | 2.15 | 4108 |
| 425094 | AI955956 | Hs.21417 | ESTs | 2.15 | 1919 |
| 453265 | U61232 | Hs.343564 | tubulin-specific chaperone e | 2.15 | 4389 5773 |
| 445255 | NM_014841 | Hs.12477 | synaptosomal-associated protein, 91 kDa | 2.15 | 3663 5653 |
| 400125 | | | Eos Control | 2.15 | |
| 456806 | AI222298 | Hs.140720 | GSK-3 binding protein FRAT2 | 2.15 | 4561 |
| 447497 | AW167254 | Hs.205722 | ESTs | 2.15 | 3869 |
| 405869 | | | Target Exon | 2.15 | |
| 406261 | | | Target Exon | 2.15 | |
| 413286 | AA127984 | Hs.222024 | transcription factor BMAL2 | 2.15 | 708 |
| 446457 | AI300580 | | ESTs, Moderately similar to ALU1_HUMAN A | 2.15 | 3752 |
| 437054 | AA743376 | Hs.120592 | ESTs | 2.15 | 3032 |
| 401708 | | | Target Exon | 2.15 | |
| 417185 | NM_002484 | Hs.81469 | nucleotide binding protein 1 (E. coli Min | 2.15 | 1070 4980 |
| 402704 | | | C1001099*: gi\|6005896\|ref\|NP_009101.1\| te | 2.15 | |
| 454923 | AW897236 | | gb: CM0-NN0057-150400-335-c06 NN0057 Homo | 2.15 | 4498 |
| 431231 | AA653552 | Hs.116532 | ESTs | 2.15 | 2542 |
| 447229 | BE617135 | Hs.22612 | hypothetical protein DKFZp566D1346 | 2.15 | 3832 |
| 416554 | H72721 | Hs.203509 | ESTs, Weakly similar to I38022 hypotheti | 2.15 | 1002 |
| 431316 | AA502663 | Hs.145037 | ESTs | 2.15 | 2548 |
| 414727 | BE466904 | Hs.190162 | gb: hz28f03.x1 NCI_CGAP_GC6 Homo sapiens | 2.15 | 870 |
| 410307 | AF022913 | Hs.62187 | phosphatidylinositol glycan, class K | 2.15 | 464 4844 |
| 409358 | AI609021 | Hs.292725 | ESTs, Weakly similar to T18818 hypotheti | 2.15 | 363 |
| 442611 | BE077155 | Hs.177537 | hypothetical protein DKFZp761B1514 | 2.15 | 3453 |
| 408653 | AW410189 | Hs.98074 | itchy (mouse homolog) E3 ubiquitin prote | 2.15 | 287 |
| 418849 | AW474547 | Hs.53565 | Homo sapiens PIG-M mRNA for mannosyltran | 2.15 | 1249 |
| 418647 | AA226198 | | gb: nc26a07.s1 NCI_CGAP_Pr1 Homo sapiens | 2.15 | 1218 |
| 456516 | BE172704 | Hs.222746 | KIAA1610 protein | 2.15 | 4548 |
| 422330 | D30783 | Hs.115263 | epiregulin | 2.15 | 1632 5147 |
| 418437 | AA771738 | Hs.348000 | ESTs, Moderately similar to ALU5_HUMAN A | 2.15 | 1201 |
| 457100 | AA417878 | Hs.48401 | ESTs, Moderately similar to ALU8_HUMAN A | 2.15 | 4578 |
| 445890 | AF055019 | Hs.21906 | Homo sapiens clone 24670 mRNA sequence | 2.15 | 3708 |
| 447957 | NM_014821 | Hs.20126 | KIAA0317 gene product | 2.15 | 3925 5703 |
| 442578 | AK001643 | Hs.8395 | hypothetical protein FLJ10781 | 2.15 | 3448 5622 |
| 400987 | | | C11000939: gi\|11464993\|ref\|NP_065260.1\| g | 2.15 | |
| 455642 | BE063965 | | gb: QV3-BT0296-140200-085-h01 BT0296 Homo | 2.15 | 4517 |
| 484029 | | | NM_018936*: Homo sapiens protocadherin be | 2.15 | 4718 50 |
| 452817 | AA322859 | Hs.284275 | Homo sapiens PAK2 mRNA, complete cds | 2.15 | 4347 |
| 423165 | AI937547 | Hs.124915 | hypothetical protein MGC2601 | 2.15 | 1722 |
| 442327 | AA991745 | Hs.42522 | ESTs | 2.15 | 3418 |
| 457146 | BE271371 | | biphenyl hydrolase-like (serine hydrolas | 2.14 | 4581 |
| 429362 | T25833 | Hs.200478 | ubiquitin-conjugating enzyme E2M (homolo | 2.14 | 2360 |
| 448748 | AI567442 | Hs.321666 | ESTs, Weakly similar to ALUF_HUMAN !!!! | 2.14 | 4002 |
| 406596 | | | C15000556*: gi\|11862941\|dbj\|BAB19279.1\|) | 2.14 | |
| 440388 | AI693520 | Hs.223000 | ESTs | 2.14 | 3286 |
| 436314 | AI983409 | | ESTs | 2.14 | 2979 |
| 447433 | AA651869 | Hs.5320 | hypothetical protein | 2.14 | 3860 |
| 433441 | R37094 | Hs.13742 | ESTs | 2.14 | 2763 |
| 458497 | AI161428 | Hs.75916 | splicing factor 3b, subunit 2, 145 kD | 2.14 | 4625 |
| 429938 | BE296804 | Hs.226377 | phosphate cytidylyltransferase 2, ethano | 2.14 | 2429 |
| 444859 | AW449137 | Hs.157487 | ESTs | 2.14 | 3634 |
| 421628 | AL121317 | Hs.106210 | hypothetical protein FLJ10813 | 2.14 | 1547 |
| 450832 | AW970602 | Hs.105421 | ESTs | 2.14 | 4171 |
| 451622 | AW139587 | Hs.30579 | Homo sapiens cDNA: FLJ23070 fis, clone L | 2.14 | 4236 |
| 409125 | R17268 | Hs.343567 | axonal transport of synaptic vesicles | 2.14 | 336 |
| 407347 | AA829847 | | gb: od40d07.s1 NCI_CGAP_GCB1 Homo sapiens | 2.14 | 154 |
| 405850 | | | Target Exon | 2.14 | |
| 437879 | BE262082 | Hs.5894 | hypothetical protein FLJ10305 | 2.14 | 3102 |
| 454515 | AW803201 | | intron of: trichorhinophalangeal syndro | 2.14 | 4487 |
| 428717 | T78001 | Hs.133546 | hypothetical protein FLJ21120 | 2.14 | 2285 |
| 410668 | BE379794 | Hs.159651 | tumor necrosis factor receptor superfami | 2.14 | 503 |
| 437673 | AW665665 | Hs.153034 | ESTs | 2.14 | 3081 |
| 440049 | R06699 | Hs.19769 | hypothetical protein MGC4174 | 2.14 | 3257 |
| 434766 | AA742222 | Hs.120634 | ESTs | 2.14 | 2865 |
| 458882 | R34993 | Hs.226666 | ESTs, Moderately similar to I54374 gene | 2.14 | 4643 |
| 432593 | AW301003 | Hs.51483 | ESTs, Weakly similar to hypothetical pro | 2.14 | 2682 |
| 458744 | AW445183 | | ESTs | 2.13 | 4634 |
| 419557 | N94706 | | Homo sapiens cDNA FLJ14028 fis, clone HE | 2.13 | 1333 |
| 413771 | AA810047 | Hs.4112 | acetyl-Coencyme A acetyltransferase 2 (a | 2.13 | 752 |
| 452679 | Z42387 | Hs.83883 | transmembrane, prostate androgen induced | 2.13 | 4333 |
| 444773 | BE156256 | Hs.11923 | hypothetical protein | 2.13 | 3627 |
| 419409 | AW297831 | Hs.143792 | hypothetical protein MGC2656 | 2.13 | 1312 |
| 415989 | AI267700 | | ESTs | 2.13 | 963 |
| 413283 | R78669 | Hs.23756 | hypothetical protein similar to swine ac | 2.13 | 707 |
| 448648 | BE614345 | Hs.297214 | ESTs, Weakly similar to ALU7_HUMAN ALU S | 2.13 | 3992 |
| 425003 | AF119046 | Hs.154149 | apurinic/apyrimidinic endonuclease(APEX | 2.13 | 1909 5242 |
| 447460 | AW872495 | | hypothetical protein FLJ12439 | 2.13 | 3863 |
| 413219 | AA878200 | Hs.118727 | Homo sapiens cDNA FLJ13692 fis, clone PL | 2.13 | 701 |
| 427550 | BE242818 | Hs.311609 | nuclear RNA helicase, DECD variant of DE | 2.13 | 2165 |
| 446627 | AI973016 | Hs.15725 | hypothetical protein SBBI48 | 2.13 | 3772 |
| 421190 | U95031 | Hs.102482 | mucin 5, subtype B, tracheobronchial | 2.13 | 1488 5096 |
| 419764 | BE262524 | Hs.93183 | vasodilator-stimulated phosphoprotein | 2.13 | 1360 |
| 432170 | T56887 | Hs.18282 | KIAA1134 protein | 2.13 | 2638 |
| 436905 | N31273 | Hs.42380 | ESTs | 2.13 | 3020 |
| 419674 | AK000170 | Hs.92254 | synaptotagmin-like 2 | 2.13 | 1347 5055 |
| 419125 | AA642452 | Hs.130881 | B-cell CLL/lymphoma 11A (zinc finger pro | 2.13 | 1279 |
| 435858 | AF254260 | Hs.283009 | tuftelin 1 | 2.13 | 2950 5572 |
| 438614 | AB037726 | Hs.288348 | KIAA1305 protein | 2.13 | 3154 5597 |
| 448889 | BE140902 | | gb: IL1-HT0028-240699-001-C11 HT0028 Homo | 2.13 | 4018 |
| 439117 | AF085975 | | gb: Homo sapiens full length insert cDNA | 2.13 | 3187 |
| 404745 | | | Target Exon | 2.13 | |
| 453205 | W92881 | Hs.117235 | ESTs | 2.13 | 4386 |
| 418340 | NM_013286 | Hs.84162 | chromosome 3p21.1 gene sequence | 2.13 | 1190 5011 |
| 413405 | AW022253 | Hs.215976 | ESTs | 2.13 | 716 |
| 418429 | AB010427 | Hs.85100 | WD repeat domain 1 | 2.13 | 1200 5016 |
| 441134 | W29092 | Hs.346950 | cellular retinoic acid-binding protein 1 | 2.13 | 3335 |
| 437277 | AA748016 | Hs.123370 | ESTs | 2.12 | 3055 |
| 409310 | R88721 | Hs.164584 | ESTs | 2.12 | 360 |
| 435905 | AW997484 | Hs.5003 | KIAA0456 protein | 2.12 | 2951 |
| 401908 | | | C17000154: gi\|12003980\|gb\|AAG43830.1\|AF21 | 2.12 | |
| 419152 | L12711 | Hs.89643 | transketolase (Wernicke-Korsakoff syndro | 2.12 | 1283 5040 |
| 418900 | BE207357 | Hs.3454 | KIAA1821 protein | 2.12 | 1258 |
| 405365 | | | CX001212*: gi\|7861932\|gb\|AAF70445.1\| (AF2 | 2.12 | |
| 413199 | M62843 | Hs.75236 | ELAV (embryonic lethal, abnormal vision, | 2.12 | 4895 699 |
| 427300 | AA400518 | Hs.169395 | hypothetical protein FLJ12015 | 2.12 | 2140 |
| 421254 | AK001724 | Hs.102950 | coat protein gamma-cop | 2.12 | 1496 |
| 424411 | NM_005209 | Hs.146549 | crystallin, beta A2 | 2.12 | 1841 5211 |
| 421458 | NM_003654 | Hs.104576 | carbohydrate (keratan sulfate Gal-6) sul | 2.12 | 1521 5107 |
| 418597 | AK001678 | Hs.86337 | similar to DNA-directed RNA polymerase I | 2.12 | 1216 5021 |
| 430539 | AK001489 | | ADP-ribosylation factor-like 1 | 2.12 | 2495 |
| 438730 | BE041332 | Hs.7307 | ESTs, Weakly similar to KT12_YEAST KTI12 | 2.12 | 3162 |
| 450202 | AW969756 | Hs.34145 | ESTs, Weakly similar to B49647 GTP-bindi | 2.12 | 4112 |
| 414792 | BE314949 | Hs.87128 | hypothetical protein FLJ23309 | 2.11 | 878 |
| 450042 | AI681099 | Hs.271596 | ESTs | 2.11 | 4099 |
| 413422 | AW176733 | Hs.3826 | kelch-like protein C3IP1 | 2.11 | 718 |
| 448529 | T26460 | Hs.22550 | ESTs | 2.11 | 3981 |
| 408665 | T88845 | Hs.112200 | ESTs, Weakly similar to ALU7_HUMAN ALU S | 2.11 | 289 |
| 444381 | BE387335 | Hs.283713 | hypothetical protein BC014245 | 2.11 | 3593 |
| 412133 | U83460 | Hs.73614 | solute carrier family 31 (copper transpo | 2.11 | 4874 612 |
| 429290 | AF203032 | Hs.198760 | neurofilament, heavy polypeptide (200 kD) | 2.11 | 2350 5398 |
| 418515 | AI568453 | Hs.19487 | ESTs, Weakly similar to CNIH_HUMAN CORNI | 2.11 | 1210 |
| 419395 | BE268326 | Hs.90280 | 5-aminoimidazole-4-carboxamide ribonucle | 2.11 | 1310 |
| 445892 | AV655500 | Hs.93961 | Homo sapiens mRNA; cDNA DKFZp667D095 (fr | 2.11 | 3709 |
| 408559 | W30787 | Hs.45105 | ESTs | 2.11 | 279 |
| 428826 | AL048842 | Hs.194019 | attractin | 2.11 | 2305 |
| 449188 | AW072939 | Hs.347187 | myotubularin related protein 1 | 2.11 | 4043 |
| 421307 | BE539976 | Hs.103305 | Homo sapiens mRNA; cDNA DKFZp434B0425 (f | 2.11 | 1506 |
| 414057 | AI815559 | Hs.75730 | signal recognition particle receptor (′d | 2.11 | 781 |
| 408162 | AA993833 | Hs.118527 | ESTs | 2.11 | 241 |
| 453028 | AB006532 | Hs.31442 | RecQ protein-like 4 | 2.10 | 4368 5768 |
| 421777 | BE562088 | Hs.108196 | HSPC037 protein | 2.10 | 1562 |
| 449568 | AL157479 | Hs.23740 | KIAA 1598 protein | 2.10 | 4067 |
| 405883 | | | Target Exon | 2.10 | |
| 401095 | | | NM_022363: Homo sapiens LIM homeobox prot | 2.10 | 24 4695 |
| 402840 | | | ENSP00000241321*: DJ947L8.1.6 (novel CUB | 2.10 | |
| 443784 | U82670 | | zinc finger protein 275 | 2.10 | 3550 |
| 432393 | AW205863 | Hs.133988 | hypothetical protein FKSG28 | 2.10 | 2663 |
| 430512 | AF182294 | Hs.241578 | U6 snRNA-associated Sm-like protein LSm8 | 2.10 | 2491 5453 |
| 434170 | AA626509 | Hs.122329 | ESTs | 2.10 | 2817 |
| 410001 | AB041036 | Hs.57771 | kallikrein 11 | 2.10 | 427 4834 |
| 429438 | AC006293 | | Homo sapiens killer cell Ig-like recepto | 2.10 | 2369 5406 |
| 456259 | Z44190 | Hs.83023 | peroxisomal biogenesis factor 11B | 2.10 | 4536 |
| 410430 | AW732554 | Hs.59622 | Homo sapiens, clone IMAGE: 3507281, mRNA, | 2.10 | 477 |
| 421052 | AA648486 | Hs.118993 | ESTs | 2.10 | 1477 |
| 412700 | BE222433 | | ESTs, Weakly similar to I38022 hypotheti | 2.10 | 660 |
| 441549 | BE563873 | Hs.124005 | ESTs, Weakly similar to T29899 hypotheti | 2.10 | 3356 |
| 418397 | NM_001269 | Hs.84746 | chromosome condensation 1 | 2.10 | 1195 5013 |
| 449249 | T52285 | Hs.193115 | Homo sapiens mRNA for KIAA1764 protein, | 2.10 | 4.50 |
| 429551 | AW450624 | Hs.220931 | ESTs | 2.10 | 2388 |
| 419677 | N77342 | Hs.21851 | Homo sapiens cDNA FLJ12900 fis, clone NT | 2.10 | 1348 |
| 408756 | AA524743 | | ESTs | 2.10 | 303 |
| 417952 | AI192838 | | dual-specificity tyrosine-(Y)-phosphoryl | 2.10 | 1141 |
| 447401 | BE618582 | Hs.97661 | ESTs | 2.10 | 3858 |
| 449721 | AW073727 | Hs.210265 | ESTs | 2.10 | 4078 |
| 406255 | | | Target Exon | 2.10 | |
| 441676 | BE564206 | Hs.49889 | ESTs | 2.10 | 3367 |
| 418050 | R37848 | Hs.7177 | ESTs | 2.10 | 1151 |
| 454631 | AW811324 | | gb: IL3-ST0141-131099-017-A02 ST0141 Homo | 2.10 | 4492 |
| 419575 | U43431 | Hs.91175 | topoisomerase (DNA) III alpha | 2.10 | 1334 5052 |
| 454171 | AW854832 | | gb: QV2-CT0261-201099-011-f05 CT0261 Homo | 2.10 | |
| 433923 | AI823453 | Hs.146625 | ESTs | 2.10 | 2798 |
| 443744 | AI084326 | Hs.271548 | ESTs, Weakly similar to I78885 serine/th | 2.10 | 3548 |
| 443713 | AI082810 | Hs.204934 | ESTs | 2.10 | 3543 |
| 418671 | D82344 | Hs.87202 | paired mesoderm homeobox 2b | 2.10 | 1224 5023 |
| 428585 | AB007863 | Hs.185140 | KIAA0403 protein | 2.10 | 2274 5366 |
| 404306 | | | Target Exon | 2.10 | |
| 433745 | AF075320 | Hs.28980 | hypothetical protein FLJ14540 | 2.10 | 2786 |
| 410232 | AW372451 | Hs.61184 | CGI-79 protein | 2.10 | 458 |
| 452335 | AW188944 | Hs.61272 | ESTs | 2.10 | 4297 |
| 454427 | AW605620 | Hs.76064 | ribosomal protein L27a | 2.10 | 4483 |
| 450963 | AI864668 | Hs.48832 | ESTs | 2.10 | 4179 |
| 427540 | R12014 | Hs.20976 | ESTs | 2.10 | 2162 |
| 406740 | AA577274 | | gb: nm85g07.s1 NCI_CGAP_Co9 Homo sapiens | 2.10 | 95 |
| 429263 | AA019004 | Hs.198396 | ATP-binding cassette, sub-family A (ABC1 | 2.10 | 2345 |
| 419485 | AA489023 | Hs.99807 | ESTs, Weakly similar to unnamed protein | 2.10 | 1318 |
| 407644 | D16815 | | nuclear receptor subfamily 1, group D, m | 2.10 | 176 4777 |
| 429058 | AF138863 | Hs.35254 | hypothetical protein FLB6421 | 2.10 | 2331 5389 |
| 410382 | AW664971 | Hs.259546 | ESTs | 2.10 | 470 |
| 431788 | AA992677 | | thymopoietin | 2.10 | 2598 |
| 419200 | AW966405 | | EST | 2.09 | 1288 |
| 436315 | BE390513 | Hs.27935 | hypothetical protein MGC4837 | 2.09 | 2980 |
| 421462 | AF016495 | Hs.104624 | aquaporin 9 | 2.09 | 1522 5108 |
| 420711 | R18398 | | ESTs | 2.09 | 1444 |
| 419667 | AU077005 | Hs.92208 | a disintegrin and metalloproteinase doma | 2.09 | 1346 |
| 412430 | AW675064 | Hs.73875 | fumarylacetoacetate hydrolase (fumarylac | 2.09 | 630 |
| 424197 | AF096834 | Hs.142989 | germ cell specific Y-box binding protein | 2.09 | 1814 5202 |
| 437623 | D63880 | Hs.5719 | chromosome condensation-related SMC-asso | 2.09 | 3076 5587 |
| 445863 | R12234 | Hs.13396 | Homo sapiens clone 25028 mRNA sequence | 2.09 | 3703 |
| 450306 | AL080080 | Hs.24766 | thioredoxin domain-containing | 2.09 | 4127 5728 |
| 412939 | AW411491 | Hs.75069 | eukaryotic translation elongation factor | 2.09 | 684 |
| 407276 | AI951118 | Hs.326736 | Homo sapiens breast cancer antigen NY-BR | 2.09 | 147 |
| 408283 | BE141579 | | gb: QV2-HT0083-071299-018-b05 HT0083 Homo | 2.09 | 251 |
| 414198 | AW505308 | Hs.75812 | phosphoenolpyruvate carboxykinase 2 (mit | 2.09 | 798 |
| 448556 | AW885606 | Hs.5064 | ESTs | 2.09 | 3985 |
| 424505 | AA446131 | Hs.124918 | KIAA1795 protein | 2.09 | 1853 |
| 426691 | NM_006201 | Hs.171834 | PCTAIRE protein kinase 1 | 2.09 | 2088 5314 |
| 448545 | BE543187 | Hs.105097 | thymidine kinase 1, soluble | 2.09 | 3982 |
| 432901 | AI554929 | Hs.281866 | ATPase, H transporting, lysosomal (vacuo | 2.09 | 2710 |
| 447976 | AW972653 | Hs.293691 | ESTs, Highly similar to CR2_HUMAN COMPLE | 2.09 | 3931 |
| 417378 | R57256 | Hs.82037 | TATA box binding protein (TBP)-associate | 2.09 | 1088 |
| 413643 | AA130987 | Hs.188727 | ESTs | 2.09 | 743 |
| 408201 | AK000568 | Hs.43654 | hypothetical protein FLJ20561 | 2.09 | 2444791 |
| 443031 | AW134096 | Hs.49418 | ESTs | 2.09 | 3488 |
| 450898 | N54911 | Hs.171765 | ESTs | 2.09 | 4173 |
| 455908 | BE156306 | | gb: QV0-HT0367-150200-114-h04 HT0367 Homo | 2.09 | 4523 |
| 404053 | | | Target Exon | 2.09 | |
| 457242 | AA457011 | Hs.80261 | gb: aa90c11.r1 Stratagene fetal retina 93 | 2.09 | 4585 |
| 430354 | AA954810 | Hs.239784 | human homolog of Drosophila Scribble | 2.09 | 2474 |
| 443119 | AA312264 | Hs.7980 | hypothetical protein MGC12966 | 2.09 | 3498 |
| 400834 | | | NM_002240*: Homo sapiens potassium inward | 2.09 | 21 4692 |
| 408338 | AW867079 | | gb: MR1-SN0033-120400-002-c10 SN0033 Homo | 2.09 | 256 |
| 426399 | AA652588 | Hs.301348 | Homo sapiens cDNA FLJ13271 fis, clone OV | 2.09 | 2061 |
| 422830 | AC007954 | Hs.121371 | hypothetical protein DKFZp434P0111 | 2.08 | 1685 5159 |
| 458676 | AI692464 | Hs.202263 | ESTs | 2.08 | 4632 |
| 417939 | R53863 | Hs.337512 | ESTs, Weakly similar to ALUB_HUMAN !!!! | 2.08 | 1140 |
| 452184 | R16281 | Hs.21701 | linked to Surfeit genes in Fugu rubripes | 2.08 | 4280 |
| 408822 | AW500715 | Hs.57079 | Homo sapiens cDNA FLJ13267 fis, clone OV | 2.08 | 308 |
| 429305 | AF095727 | Hs.287832 | myelin protein zero-like 1 | 2.08 | 2353 5400 |
| 419355 | AA428520 | Hs.90061 | progesterone binding protein | 2.08 | 1304 |
| 447887 | AA114050 | Hs.19949 | caspase 8, apoptosis-related cysteine pr | 2.08 | 3918 |
| 421443 | BE550141 | Hs.156148 | hypothetical protein FLJ13231 | 2.08 | 1520 |
| 451422 | AB002336 | Hs.26395 | erythrocyte membrane protein band 4.1-li | 2.08 | 4222 5744 |
| 423797 | BE259364 | Hs.132898 | fatty acid desaturase 1 | 2.08 | 1775 |
| 401192 | | | Target Exon | 2.08 | |
| 440028 | AW473675 | | ESTs, Weakly similar to T17227 hypotheti | 2.08 | 3253 |
| 449769 | AI668700 | Hs.226564 | ESTs, Moderately similar to ALU6_HUMAN A | 2.08 | 4081 |
| 450330 | AW500775 | Hs.24817 | hypothetical protein FLJ20136 | 2.08 | 4130 |
| 453518 | AW503205 | Hs.27268 | gb: UI-HF-BN0-akt-g-03-0-UI.r1 NIH_MGC_50 | 2.08 | 4417 |
| 420750 | AW190215 | Hs.62348 | hypothetical protein FLJ11753 | 2.08 | 1449 |
| 434033 | AI631749 | Hs.156616 | ESTs, Weakly similar to alternatively sp | 2.08 | 2807 |
| 425001 | U55184 | Hs.154145 | hypothetical protein FLJ11585 | 2.08 | 1908 5241 |
| 414809 | AI434699 | Hs.77356 | transferrin receptor (p90, CD71) | 2.08 | 880 |
| 411580 | AL080088 | Hs.70877 | DKFZP564K2062 protein | 2.08 | 4865 561 |
| 432458 | AI968598 | Hs.78768 | malignant cell expression-enhanced gene/ | 2.08 | 2669 |
| 459646 | AW883968 | Hs.321190 | gb: QV3-OT0063-290300-135-c04 OT0063 Homo | 2.08 | 4674 |
| 434350 | AL042940 | Hs.93872 | KIAA1682 protein | 2.08 | 2831 |
| 436610 | AW611912 | Hs.98751 | ESTs | 2.08 | 2998 |
| 413045 | X92121 | Hs.75180 | protein phosphatase 5, catalytic subunit | 2.08 | 4892 691 |
| 416855 | AA188763 | Hs.36793 | hypothetical protein FLJ23188 | 2.08 | 1032 |
| 423137 | NM_007212 | Hs.124186 | ring finger protein 2 | 2.08 | 1721 5171 |
| 427239 | BE270447 | | ubiquitin carrier protein | 2.08 | 2134 |
| 426526 | BE617944 | Hs.170226 | Homo sapiens clone 23579 mRNA sequence | 2.07 | 2075 |
| 411619 | AI418609 | Hs.71040 | hypothetical protein FLJ20425 | 2.07 | 564 |
| 420762 | U51699 | | dolichyt-phosphate (UDP-N-acetylglucosam | 2.07 | 1451 |
| 457133 | M54968 | | v-Ki-ras2 Kirsten rat sarcoma 2 viral on | 2.07 | 4580 5811 |
| 414581 | AA256213 | Hs.72010 | ESTs | 2.07 | 849 |
| 430750 | AI650360 | Hs.100256 | ESTs | 2.07 | 2511 |
| 405760 | | | Target Exon | 2.07 | |
| 436862 | AI821940 | Hs.116567 | ESTs, Moderately similar to ALU8_HUMAN A | 2.07 | 3013 |
| 453690 | AI674872 | Hs.107000 | hypothetical protein FLJ11294 | 2.07 | 4424 |
| 435750 | AB029012 | Hs.4990 | KIAA1089 protein | 2.07 | 2939 5570 |
| 428977 | AK001404 | Hs.194698 | cyclin B2 | 2.07 | 2323 |
| 431188 | W05656 | Hs.169755 | ESTs | 2.07 | 2538 |
| 410407 | X66839 | Hs.63287 | carbonic anhydrase IX | 2.07 | 474 4846 |
| 418874 | T60872 | | gb: yb72h11.s1 Stratagene ovary (937217) | 2.07 | 1253 |
| 416305 | AU076628 | Hs.79187 | coxsackie virus and adenovirus receptor | 2.07 | 985 |
| 441252 | AW360901 | | hypothetical protein MGC4399 | 2.07 | 3341 |
| 420596 | NM_002692 | Hs.99185 | polymerase (DNA directed), epsilon 2 | 2.07 | 1435 5082 |
| 408546 | W49512 | Hs.46348 | bradykinin receptor B1 | 2.07 | 277 |
| 404071 | | | C12000514*: gi\|7302471\|gb\|AAF57556.1\|(AE | 2.07 | |
| 408946 | AW854991 | Hs.255565 | ESTs | 2.07 | 318 |
| 420614 | AL110291 | Hs.99364 | putative transmembrane protein | 2.07 | 1438 |
| 404754 | | | C3001259: gi\|476355\|pir\|\|A46762 myosin al | 2.07 | 61 |
| 424055 | AA337234 | | gb: EST42299 Endometrial tumor homo sapie | 2.06 | 1797 |
| 434875 | AW974785 | Hs.290831 | ESTs | 2.06 | 2877 |
| 418793 | AW382987 | Hs.88474 | prostaglandin-endoperoxide synthase 1 (p | 2.06 | 1240 |
| 405710 | | | CX000682: gi\|12741327\|reflHP_008833.2\| zi | 2.06 | |
| 421604 | AW293880 | Hs.248367 | MEGF11 protein | 2.06 | 1542 |
| 447370 | AW248150 | Hs.18349 | mitochondrial ribosomal protein L15 | 2.06 | 3850 |
| 411800 | N39342 | Hs.103042 | microtubule-associated protein 1B | 2.06 | 579 |
| 442993 | BE018682 | Hs.166196 | ATPase, Class I type 8B, member 1 | 2.06 | 3486 |
| 434075 | AW003416 | Hs.160604 | ESTs | 2.06 | 2813 |
| 428583 | AA430589 | Hs.75410 | heat shock 70 kD protein 5 (glucose-regul | 2.06 | 2273 |
| 422975 | AA347720 | Hs.122669 | KIAA0264 protein | 2.06 | 1704 |
| 451273 | NM_014811 | Hs.26163 | KIAA0649 gene product | 2.06 | 4206 5741 |
| 433399 | N46406 | Hs.84700 | similar to phosphatidylcholine transfer | 2.06 | 2760 |
| 431849 | AI670823 | Hs.85573 | hypothetical protein MGC10911 | 2.06 | 2607 |
| 420324 | AF163474 | Hs.96744 | prostate androgen-regulated transcript 1 | 2.06 | 1412 5074 |
| 443677 | AV646096 | Hs.282990 | ESTs, Weakly similar to B34087 hypotheti | 2.06 | 3537 |
| 405779 | | | NM_005367: Homo sapiens melanoma antigen, | 2.06 | 4741 75 |
| 439357 | AW955708 | Hs.100651 | golgi SNAP receptor complex member 2 | 2.06 | 3203 |
| 400880 | | | NM_000611*: Homo sapiens CD59 antigen p18 | 2.05 | 23 4694 |
| 415079 | R43179 | Hs.22895 | hypothetical protein FLJ23548 | 2.05 | 906 |
| 415788 | AW628686 | Hs.78851 | KIAA0217 protein | 2.05 | 952 |
| 405697 | | | gb: Human homeobox-like mRNA | 2.05 | |
| 431441 | U81961 | Hs.2794 | sodium channel, nonvoltage-gated 1 alpha | 2.05 | 2554 5475 |
| 408086 | AW351543 | Hs.42514 | hypothetical protein F25965 | 2.05 | 232 |
| 458335 | AI762479 | | gb: wh91a04x1 NCl_CGAP_CLL1 Homo sapiens | 2.05 | 4618 |
| 401898 | | | NM_024722*: Homo sapiens hypothetical pro | 2.05 | 30 4700 |
| 425255 | AW374106 | Hs.155356 | hypothetical protein MGC2840 similar to | 2.05 | 1944 |
| 413976 | BE295452 | Hs.75655 | procollagen-proline, 2-oxolgutate 4-di | 2.05 | 771 |
| 421937 | AI878857 | Hs.109706 | hematological and neurological expressed | 2.05 | 1582 |
| 426329 | AL389951 | Hs.271623 | nucleporin 50 kD | 2.05 | 2054 5300 |
| 445243 | AI217439 | | ESTs, Weakly similar to ALU1_HUMAN ALU S | 2.05 | 3662 |
| 451930 | BE259124 | Hs.27262 | Homo sapiens clone 25110 mRNA sequence | 2.05 | 4257 |
| 431846 | BE019924 | Hs.271580 | uroplakin 1B | 2.05 | 2605 |
| 410847 | AW807090 | | gb: MR4-ST0062-031199-018-d12 ST0062 Homo | 2.05 | 517 |
| 449523 | NM_000579 | Hs.54443 | chemokline (C-C motif) receptor 5 | 2.05 | 4064 5721 |
| 411051 | AW853931 | | gb: RC3-CT0254-180200-026-e04 CT0254 Homo | 2.05 | 531 |
| 418967 | NM_001725 | Hs.89535 | bactericidal/permeability-increasing pro | 2.05 | 1265 5035 |
| 408841 | AW438865 | Hs.256862 | ESTs | 2.05 | 312 |
| 403509 | | | KIAA0539 gene product | 2.05 | |
| 418898 | T66847 | Hs.194040 | ESTs, Weakly similar to I38022 hypotheti | 2.05 | 1257 |
| 453438 | AI469935 | Hs.22792 | ESTs | 2.05 | 4405 |
| 443084 | AI27639 | Hs.125539 | ESTs | 2.05 | 3495 |
| 410656 | BE161335 | Hs.321717 | ESTs, Weakly similar to S22765 heterogen | 2.05 | 501 |
| 441879 | AI1521936 | Hs.107149 | novel protein similar to archaeal, yeast | 2.05 | 3377 |
| 453081 | AI951203 | Hs.223345 | ESTs | 2.05 | 4374 |
| 455211 | AW866449 | | gb: QV4-SN0024-170400-176-a12 SN0024 Homo | 2.05 | 4502 |
| 448824 | L41147 | Hs.22180 | 5-hydroxytyptamine (serotonin) receptor | 2.05 | 4011 5714 |
| 418319 | AW611703 | Hs.190173 | ESTs, Weakly similar to A46010 X-linked | 2.05 | 1183 |
| 442317 | AI915599 | Hs.129225 | ESTs | 2.05 | 3415 |
| 400249 | | | Eos Control | 2.05 | |
| 439092 | AA830149 | | gb: oc44f08.s1 NCl_CGAP_GCB1 Homo sapiens | 2.05 | 3185 |
| 448390 | AL035414 | Hs.21068 | hypothetical protein | 2.05 | 3963 |
| 422661 | NM_014700 | Hs.119004 | KIAA0665 gene product | 2.05 | 1669 5156 |
| 444682 | R37390 | Hs.21119 | ESTs | 2.05 | 3619 |
| 428315 | AA688152 | Hs.98505 | ESTs | 2.05 | 2239 |
| 440953 | AI683036 | Hs.124135 | Homo sapiens cDNA FLJ13051 fis, clone NT | 2.05 | 3321 |
| 427178 | AA398866 | Hs.97542 | Homo sapiens testis-development related | 2.05 | 2130 |
| 414212 | AA136569 | Hs.10848 | KIAA0187 gene product | 2.05 | 800 |
| 415892 | H08267 | Hs.302330 | ESTs, Moderately similar to JC5238 galac | 2.05 | 958 |
| 424624 | AB032947 | Hs.151301 | Ca2+dependent activator protein for secr | 2.05 | 1864 5220 |
| 454838 | AW833984 | | gb: QV0-TT0010-031199-045-a05 TT0010 Homo | 2.05 | 4497 |
| 414709 | AA704703 | Hs.77031 | Sp2 transcription factor | 2.05 | 866 |
| 401463 | | | histone deacetylase 5 | 2.05 | |
| 453849 | N35321 | Hs.278611 | UDP-N-acetyl-alpha-D-galactosamine: polyp | 2.05 | 4436 |
| 453344 | BE349075 | Hs.44571 | ESTs | 2.05 | 4397 |
| 431790 | AA650250 | Hs.272076 | ESTs | 2.05 | 2600 |
| 457898 | AI382260 | Hs.129943 | ESTs, Weakly similar to high-risk human | 2.05 | 4604 |
| 443423 | AW162357 | Hs.7023 | ESTs | 2.06 | 3522 |
| 426172 | AA371307 | Hs.125056 | ESTs | 2.06 | 2035 |
| 423871 | AA331906 | Hs.175596 | gb: EST35805 Embryo, 8 week I Homo sapien | 2.05 | 1783 |
| 432146 | AW081072 | Hs.115960 | KIAA0939 protein | 2.05 | 2635 |
| 422906 | U80773 | Hs.121580 | Human EST clone 42944 mariner transposon | 2.05 | 1691 |
| 444822 | BE164351 | Hs.292767 | hypothetical protein FLJ23109 | 2.05 | 3631 |
| 422273 | AA307796 | Hs.269548 | ESTs | 2.05 | 1624 |
| 418690 | AK000052 | Hs.87293 | hypothetical protein FLJ20045 | 2.05 | 1228 5026 |
| 400730 | | | Target Exon | 2.05 | |
| 429128 | AA446869 | Hs.119316 | ESTs | 2.05 | 2333 |
| 416732 | H81066 | Hs.285017 | hypothetical protein FLJ21799 | 2.05 | 1017 |
| 423248 | AA380177 | Hs.125845 | ribulose-5-phosphate-3-epimerase | 2.05 | 1731 |
| 420544 | AA677577 | Hs.98732 | Homo sapiens Chromosome 16 BAC clone CIT | 2.05 | 1429 |
| 427356 | AW023482 | Hs.97849 | ESTs | 2.05 | 2147 |
| 435147 | AL133731 | Hs.4774 | Homo sapiens mRNA; cDNA DKFZp761C1712 (f | 2.05 | 2900 |
| 440184 | AB002297 | Hs.7022 | dedicator of cyto-kinesis 3 | 2.05 | 3269 5604 |
| 415025 | AW207091 | Hs.72307 | ESTs | 2.05 | 902 |
| 409205 | AI952884 | Hs.14832 | ESTs, Moderately similar to unnamed prot | 2.05 | 351 |
| 437672 | AW748265 | Hs.5741 | flavohemoprotein b5? | 2.05 | 3080 |
| 413916 | N49813 | Hs.75615 | apolipoprotein C-II | 2.05 | 763 |
| 403794 | | | Target Exon | 2.05 | |
| 437287 | AA748180 | Hs.159346 | hypothetical protein FLJ21369 | 2.05 | 3056 |
| 435542 | AA687376 | | ESTs | 2.05 | 2925 |
| 444391 | AL137597 | Hs.11114 | hypothetical protein dJ1181N3.1 | 2.05 | 3595 |
| 408353 | BE439838 | Hs.44298 | mitochondrial ribosomal protein S17 | 2.05 | 259 |
| 428865 | BE544095 | Hs.164960 | BarH-like homeobox 1 | 2.05 | 2314 |
| 421282 | AA286914 | Hs.40782 | ESTs | 2.05 | 1502 |
| 431250 | BE264649 | Hs.251377 | taxol resistance associated gene 3 | 2.05 | 2544 |
| 452206 | AW340281 | Hs.33074 | Homo sapiens, close IMAGE: 3606519, mRNA, | 2.05 | 4281 |
| 435730 | AB020635 | Hs.4984 | KIAA0828 protein | 2.04 | 2938 5569 |
| 404496 | | | Target Exon | 2.04 | |
| 449378 | AW664026 | Hs.59892 | ESTs | 2.04 | 4059 |
| 447528 | AI612027 | Hs.76277 | Homo sapiens, clone MGC: 9381, mRNA, comp | 2.04 | 3874 |
| 412276 | BE262621 | Hs.73798 | macrophage migration inhibitory factor ( | 2.04 | 621 |
| 400547 | | | C10000695: gi\|3153229\|gb\|AAC17225.1\| (AF0 | 2.04 | |
| 454279 | AW295650 | Hs.255453 | ESTs, Weakly similar to ALU1_HUMAN ALU S | 2.04 | 4474 |
| 435784 | AA705437 | | ESTs | 2.04 | 2943 |
| 452731 | AW451468 | Hs.257455 | ESTs | 2.04 | 4338 |
| 456381 | AA236606 | | gb: zr99b10.r1 NCI_CGAP_GCB1 Homo sapiens | 2.04 | 4540 |
| 436028 | AA731124 | Hs.15430 | ESTs | 2.04 | 2957 |
| 417169 | R13550 | Hs.21388 | ESTs | 2.04 | 1066 |
| 450052 | AI681298 | Hs.236524 | ESTs | 2.04 | 4100 |
| 412520 | AA442324 | Hs.795 | H2A histone family, member O | 2.04 | 642 |
| 456756 | AI494486 | Hs.148767 | ESTs | 2.04 | 4559 |
| 416680 | AW245540 | Hs.79516 | brain abundant, membrane attached signal | 2.04 | 1013 |
| 417630 | D63877 | Hs.82324 | KIAA0157 protein | 2.04 | 1108 4992 |
| 409617 | BE003760 | Hs.55209 | Homo sapiens mRNA; cDNA DKFZp434K0514 (f | 2.04 | 387 |
| 440603 | AL121733 | Hs.7299 | Novel human gene mapping to chomosome 1 | 2.04 | 3304 5610 |
| 412636 | NM_004415 | | desmoplakin (DPI, DPII) | 2.04 | 4882 652 |
| 454516 | AW803341 | | gb: IL2-UM0079-090300-050-D03 UM0079 Homo | 2.04 | 4488 |
| 414054 | BE244328 | Hs.288539 | hypothetical protein FLJ22191 | 2.04 | 780 |
| 419058 | AW675039 | Hs.1227 | aminolevulinate, delta-, dehydratase | 2.04 | 1271 |
| 422565 | BE259035 | Hs.118400 | singed (Drosophila)-like (sea urchin fas | 2.04 | 1657 |
| 447782 | AI668903 | | ESTs | 2.04 | 3906 |
| 413645 | AA130992 | | gb: zo15e02.s1 Stratagene colon (937204) | 2.04 | 744 |
| 456534 | X91195 | Hs.100623 | phospholipase C, beta 3, neighbor pseudo | 2.04 | 4549 |
| 433747 | AI004012 | Hs.143752 | ESTs | 2.04 | 2787 |
| 433071 | BE150229 | Hs.281564 | retinal outer segment membrane protein 1 | 2.04 | 2731 |
| 410840 | AW806924 | | gb: QV4-ST0023-160400-172-h10 ST0023 Homo | 2.04 | 516 |
| 425304 | AA463844 | Hs.31339 | fibroblast growth factor 11 | 2.04 | 1949 |
| 447711 | AI459554 | Hs.161286 | ESTs | 2.04 | 3895 |
| 449226 | AB002365 | Hs.23311 | KIAA0367 protein | 2.04 | 4047 5720 |
| 411957 | AW246216 | Hs.32058 | Homo sapiens C1orf19 mRNA, partial cds | 2.04 | 593 |
| 423226 | AA323414 | Hs.146109 | ESTs, Weakly similar to T28937 hypotheti | 2.04 | 1729 |
| 436045 | AB037723 | Hs.5028 | DKFZP564O0423 protein | 2.04 | 2959 5574 |
| 417433 | BE270266 | Hs.82128 | 5T4 oncofetal trophoblast glycoprotein | 2.04 | 1094 |
| 424744 | AW175781 | Hs.152720 | M-phase phosphoprotein 6 | 2.03 | 1881 |
| 437478 | AL390172 | Hs.317432 | branched chain aminotransferase 1, cytos | 2.03 | 3067 |
| 445003 | AI204593 | Hs.148698 | ESTs | 2.03 | 3644 |
| 432674 | AA641092 | Hs.257339 | ESTs, Weakly similar to I38022 hypotheti | 2.03 | 2690 |
| 431763 | NM_005136 | Hs.268538 | potassium voltage-gated channel, Isk-rel | 2.03 | |
| 435647 | AI653240 | Hs.49823 | ESTs | 2.03 | 2934 |
| 419341 | N71463 | Hs.118888 | ESTs, Weakly similar to ALU1_HUMAN ALU S | 2.03 | 1302 |
| 414486 | AW305026 | Hs.193974 | ESTs, Weakly similar to ALU1_HUMAN ALU S | 2.03 | 836 |
| 430383 | AI861854 | Hs.210778 | hypothetical protein FLJ10989 | 2.03 | 2478 |
| 423994 | X01057 | Hs.1724 | interleukin 2 receptor, alpha | 2.03 | 1787 5192 |
| 430828 | AI763257 | Hs.86327 | Homo sapiens cDNA: FLJ22431 fis, clone H | 2.03 | 2517 |
| 438307 | AB011093 | Hs.6150 | hypothetical protein MGC15913 | 2.03 | 3131 5594 |
| 419904 | AA974411 | Hs.18672 | ESTs | 2.03 | 1368 |
| 412017 | AA812143 | Hs.23767 | hypothetical protein FLJ12666 | 2.03 | 603 |
| 429671 | BE379335 | Hs.211594 | proteasome (prosome, macropain) 26S subu | 2.03 | 2405 |
| 437258 | AL041243 | Hs.174104 | ESTs | 2.03 | 3050 |
| 437103 | AA424881 | Hs.256301 | hypothetical protein MGC13170 | 2.03 | 124 |
| 439930 | AI218704 | Hs.144975 | ESTs | 2.03 | 3244 |
| 458207 | T28472 | Hs.7655 | U2 small nuclear ribonucleoprotein auxil | 2.03 | 4615 |
| 452882 | AW972990 | Hs.196270 | folate transporter/carrrier | 2.03 | 4357 |
| 406542 | | | C19000728*: gi\|12585552\|sp\|Q9Y2Q1\|Z257_HU | 2.03 | |
| 439941 | AI392640 | Hs.18272 | amino acid transporter system A1 | 2.03 | 3246 |
| 452277 | AL049013 | Hs.28783 | KIAA1223 protein | 2.03 | 4292 |
| 441959 | BE536998 | Hs.200360 | Homo sapiens cDNA FLJ13027 fis, clone NT | 2.03 | 3382 |
| 424899 | AL119387 | Hs.119062 | ESTs | 2.03 | 1897 |
| 418683 | U90908 | Hs.87241 | hypothetical protein from clones 23549 a | 2.03 | 1227 5025 |
| 437818 | AA769054 | | gb: oa74a08.s1 NCI_CGAP_GCB1 Homo sapiens | 2.03 | 3094 |
| 433068 | NM_006456 | Hs.288215 | sialyltransferase | 2.03 | 2730 5526 |
| 418057 | NM_012151 | Hs.83363 | coagulation factor VIII-associated (intr | 2.03 | 1153 5002 |
| 439564 | W77911 | Hs.110006 | ESTs | 2.03 | 3216 |
| 424287 | AL133105 | Hs.144633 | hypothehticat protein DKFZp434F2322 | 2.03 | 1825 5208 |
| 424075 | AI807320 | Hs.227630 | RE1-silencing transcription factor | 2.03 | 1799 |
| 426746 | J03626 | Hs.2057 | uridine monophosphate synthetase (orotat | 2.03 | 2092 5317 |
| 421690 | AW162667 | Hs.106857 | calbindin 2, (29 kD, calretinin) | 2.02 | 1554 |
| 426096 | D87436 | Hs.166318 | lipin 2 | 2.02 | 2025 5290 |
| 421626 | AI739285 | Hs.153959 | hypothetical protein MGC15436 | 2.02 | 1546 |
| 439768 | AI337300 | Hs.173138 | hypothetical protein MGC4604 | 2.02 | 3233 |
| 459001 | AI761313 | Hs.204605 | ESTs | 2.02 | 4651 |
| 451044 | AL117665 | Hs.25882 | DKFZP586M1824 protein | 2.02 | 4185 5737 |
| 448262 | AW880830 | Hs.186273 | Homo sapiens quiescin Q6 (QSCN6) | 2.02 | 3953 |
| 425256 | BE297611 | Hs.155392 | collapsin response mediator protein 1 | 2.02 | 1945 |
| 409632 | W74001 | Hs.55279 | serine (or cysteine) proteinase inhibito | 2.02 | 390 |
| 414045 | NM_002951 | Hs.75722 | ribophorin II | 2.02 | 4911 777 |
| 410072 | BE384447 | Hs.16034 | hypothetical protein MGC13186 | 2.02 | 439 |
| 408212 | AA297567 | Hs.43728 | hypothetical protein | 2.02 | 245 |
| 452732 | BE300078 | Hs.80449 | Homo sapiens, clone IMAGE: 3535294, mRNA, | 2.02 | 4339 |
| 436251 | BE515065 | Hs.296585 | nucleolar protein (KKE/D repeat) | 2.02 | 2971 |
| 422532 | AL008726 | Hs.118126 | protective protein for beta-galactosidas | 2.02 | 1653 5153 |
| 441297 | AW403084 | Hs.7766 | ubiquitin-conjugating enzyme E2E 1 (Homo | 2.02 | 3344 |
| 450649 | NM_001429 | Hs.25272 | E1A binding protein p300 | 2.02 | 4143 5732 |
| 432673 | AB028859 | Hs.278605 | DnaJ (Hsp40) homolog, subfamily B, membe | 2.02 | 2689 5510 |
| 424996 | AF006005 | Hs.154104 | pleiomorphic adenoma gene-like 2 | 2.01 | 1906 5239 |
| 418514 | AW068487 | Hs.25413 | TOLLIP protein | 2.01 | 1209 |
| 406687 | M31126 | | matrix metalloproteinase 11 (stromelysin | 2.01 | 4747 85 |
| 400133 | | | Eos Control | 2.01 | |
| 449701 | AW952323 | Hs.129908 | KIAA0591 protein | 2.01 | 4075 |
| 401405 | | | Target Exon | 2.01 | |
| 434042 | AI589941 | Hs.8254 | Homo sapiens, Similar to tumor different | 2.01 | 2808 |
| 408618 | AK000637 | Hs.46624 | HSPC043 protein | 2.01 | 284 |
| 447757 | AA071276 | Hs.19469 | KIAA0859 protein | 2.01 | 3901 |
| 432221 | M21191 | Hs.273415 | aldolase A, fructose-bisphosphate | 2.01 | 2644 |
| 400219 | | | Eos Control | 2.01 | |
| 400845 | | | NM_003105*: Homo sapiens sortilin-retated | 2.01 | 22 4693 |
| 412247 | AF022375 | Hs.73793 | vascular endothelial growth factor | 2.01 | 4876 620 |
| 413248 | T64858 | Hs.26966 | hypothetical protein DKFZp547J036 | 2.01 | 703 |
| 419663 | AA394208 | Hs.92198 | calcium-regulated heat-stable protein (2 | 2.00 | 1345 |
| 444418 | AL034417 | Hs.11169 | Gene 33/Mig-6 | 2.00 | 3596 |
| 414653 | M24486 | Hs.76768 | procollagen-proline, 2-oxoglutarate 4-di | 2.00 | 4922 858 |
| 425157 | NM_006227 | Hs.283007 | phospholipid transfer protein | 2.00 | 1930 5248 |
| 446160 | AW392197 | Hs.218003 | ESTs | 2.00 | 3728 |
| 444042 | NM_004915 | Hs.10237 | ATP-binding cassette, sub-family G (WHIT | 2.00 | 3570 5634 |
| 456895 | AA354771 | Hs.43047 | Homo sapiens cDNA FLJ13585 fis, clone PL | 2.00 | 4565 |
| 458421 | AI279978 | Hs.22547 | ESTs | 2.00 | 4623 |
| 412999 | BE046255 | | gb: hn38g10.x2 NCI_CGAP_RDF2 Homo sapiens | 2.00 | |
| 458984 | AA011185 | Hs.221189 | hypothetical protein FLJ14431 | 2.00 | 4649 |
| 444601 | AV650521 | Hs.282449 | ESTs, Moderately similar to ZN91_HUMAN Z | 2.00 | 3612 |
| 403935 | | | Target Exon | 2.00 | |
| 421258 | AA286731 | | gb: zs53d08.r1 NCI_CGAP_GCB1 Homo sapiens | 2.00 | 1498 |
| 409628 | AB021865 | Hs.55276 | potassium voltage-gated channel, Shal-re | 2.00 | 389 4829 |
| 404705 | | | Target Exon | 2.00 | |
| 404592 | | | NM_022739*: Homo sapiens E3 ubiquitin lig | 2.00 | 4725 58 |
| 456425 | AA423846 | Hs.15220 | zinc finger protein 106 | 2.00 | 4541 |
| 429731 | AK001592 | Hs.212172 | beta-carotene 15,15′-dioxygenase | 2.00 | 2411 5423 |
| 454363 | AW816274 | Hs.250154 | hypothetical protein FLJ12973 | 2.00 | 4479 |
| 414390 | BE281040 | | gb: 601156234F1 NIH_MGC_21 Homo sapiens c | 2.00 | 820 |
| 458215 | AA928160 | | gb: on86f10.s1 Soares_NFL_T_GBC_S1 Homo s | 2.00 | 4616 |
| 447500 | AI381900 | Hs.159212 | ESTs | 2.00 | 3870 |
| 445790 | AV655170 | Hs.49015 | chromosome 21 open reading frame 35 | 2.00 | 3698 |
| 445511 | AA846512 | | Homo sapiens cDNA FLJ14459 fis, clone HE | 2.00 | 3679 |
| 402239 | | | Target Exon | 2.00 | |
| 448798 | Z28671 | Hs.58606 | SNRPN upstream reading frame | 2.00 | 4008 |
| 437829 | AI358522 | Hs.103834 | ESTs | 2.00 | 3095 |
| 452470 | AI903246 | | gb: RC-BT029-070199-024 BT029 Homo sapien | 2.00 | 4313 |
| 439932 | AI589851 | | ESTs | 2.00 | 3245 |
| 425146 | AW954627 | Hs.48794 | gb: EST366697 MAGE resequences, MAGC Homo | 2.00 | 1928 |
| 417609 | AI189754 | Hs.86320 | ESTs | 2.00 | 1106 |
| 430838 | N46664 | Hs.169395 | hypothetical protein FLJ12015 | 2.00 | 2518 |
| 454525 | BE280421 | Hs.94499 | ESTs | 2.00 | 4489 |
| 454607 | AW809845 | Hs.288568 | hypothetical protein FLJ22644 | 2.00 | 4491 |
| 449016 | BE615854 | | gb: 601279735F1 NIH_MGC_39 Homo sapiens c | 2.00 | 4027 |
| 402889 | | | ENSP00000217123*: FLJ00118 protein (Fragm | 2.00 | |
| 427696 | R39126 | Hs.8038 | ESTs | 2.00 | 2183 |
| 423077 | Z78283 | | gb: Z78283 Homo sapiens brain fetus Homo | 2.00 | 1716 |
| 441373 | AI266421 | Hs.120179 | Homo sapiens cDNA: FLJ22133 fis, clone H | 2.00 | 3348 |
| 434328 | BE564937 | Hs.15984 | pp21 homolog | 2.00 | 2829 |
| 443826 | AI214805 | Hs.27232 | ESTs | 2.00 | 3556 |
| 443273 | AI042063 | Hs.132156 | ESTs | 2.00 | 3515 |
| 452999 | AI755080 | Hs.26481 | SBBI26 protein | 2.00 | 4364 |
| 451684 | AF216751 | Hs.26813 | CDA14 | 2.00 | 4240 5747 |
| 430707 | AF082350 | Hs.247820 | bone morphogenetic protein 15 | 2.00 | 2506 5462 |
| 408006 | H57654 | Hs.303345 | ESTs, Weakly similar to I38022 hypotheti | 2.00 | 224 |
| 400750 | | | Target Exon | 2.00 | |
| 419321 | N48146 | Hs.269069 | ESTs, Weakly similar to I38022 hypotheti | 2.00 | 1300 |
| 459234 | AI940425 | | gb: CM0-CT0052-150799-024-c04 CT0052 Homo | 2.00 | 4660 |
| 436277 | R88520 | Hs.120917 | ESTs | 2.00 | 2973 |
| 451106 | BE382701 | Hs.25960 | N-MYC oncogene | 2.00 | 4193 |
| 418284 | AW948651 | Hs.26002 | LIM domain binding 1 | 2.00 | 1176 |
| 413402 | T24065 | | gb: seq2245 HMSWMYK Homo sapiens cDNA clo | 2.00 | 715 |
| 438701 | AA937112 | Hs.207788 | ESTs | 2.00 | 3158 |
| 434481 | AF143330 | Hs.12621 | Homo sapiens clone IMAGE: 112577 mRNA seq | 2.00 | 2843 |
| 415121 | D60971 | Hs.34955 | Homo sapiens cDNA FLJ13485 fis, clone PL | 2.00 | 908 |
| 432492 | AW275110 | | ESTs | 2.00 | 2673 |
| 440772 | AA905574 | Hs.197444 | ESTs | 2.00 | 3317 |
| 458342 | AI003931 | Hs.334583 | ring finger protein 23 | 2.00 | 4619 |
| 453666 | AW015681 | Hs.135229 | ESTs, Weakly similar to A2BP_HUMAN ATAXI | 2.00 | 4422 |
| 409988 | N27687 | Hs.334334 | transcription factor AP-2 alpha (activat | 2.00 | 426 |
| 444430 | AI611153 | Hs.6093 | Homo sapiens cDNA: FLJ22783 fis, clone K | 2.00 | 3597 |
| 458422 | AI344782 | | DnaJ (Hsp40) homolog, subfamily C, membe | 2.00 | 4624 |
| 434636 | AA083764 | | hypothetical protein MGC3178 | 2.00 | 2856 |
| 408504 | AW205908 | Hs.73614 | ESTs, Weakly similar to 2109260A B cell | 2.00 | 274 |
| 418403 | D86978 | Hs.84790 | KIAA0225 protein | 2.00 | 1197 5015 |
| 418496 | AI564857 | Hs.27888 | ESTs, Weakly similar to serine/threonine | 2.00 | 1207 |
| 416742 | R38644 | Hs.248420 | ESTs, Moderately similar to A47582 B-cel | 2.00 | 1019 |
| 442333 | AI650877 | Hs.129302 | ESTs | 2.00 | 3422 |
| 425147 | AW291265 | Hs.334636 | hypothetical protein MGC2615 | 2.00 | 1929 |
| 415642 | U19878 | Hs.336224 | transmembrane protein with EGF-like and | 2.00 | 4951 941 |
| 414684 | AW630023 | Hs.76893 | 3-hydroxybutyrate dehydrogenase (heart, | 2.00 | 863 |
| 453354 | W55946 | Hs.234863 | Homo sapiens cDNA FLJ12082 fis, clone HE | 2.00 | 4399 |
| 442007 | AA301116 | Hs.142838 | nucleolar phosphoprotein Nopp34 | 2.00 | 3387 |
| 434326 | NM_005619 | Hs.3803 | reticulon 2 | 2.00 | 2828 5546 |
| 456487 | AF064804 | | suppressor of Ty (S. cerevisiae) 3 homolo | 2.00 | 4545 5799 |
| 443767 | BE562136 | Hs.9736 | proteasome (prosome, macropain) 26S subu | 2.00 | 3549 |
| 414569 | AF109298 | Hs.118258 | prostate cancer associated protein 1 | 2.00 | 845 |
| 419829 | AI924228 | Hs.115185 | ESTs, Moderately similar to PC4259 ferri | 2.00 | 1362 |
| 448360 | AL117560 | Hs.306352 | Homo sapiens mRNA; cDNA DKFZp566P2324 (f | 2.00 | 3959 |
| 410025 | BE220489 | Hs.113592 | ESTs, Moderately similar I54374 gene | 2.00 | 432 |
| 436961 | AW375974 | Hs.156704 | ESTs | 2.00 | 3023 |
| 443823 | BE089782 | Hs.9877 | hypothetical protein | 2.00 | 3555 |
| 436887 | AW953157 | Hs.193235 | hypothetical protein DKFZp547D155 | 2.00 | 3018 |
| 421808 | AK000157 | Hs.108502 | hypothetical protein FLJ20150 | 2.00 | 1565 5122 |
| 418375 | NM_003081 | Hs.84389 | synaptosomal-associated protein, 25 kD | 2.00 | 1192 5012 |
| 446565 | D13757 | Hs.311 | phosphoribosyl pyrophosphate amidotransf | 2.00 | 3763 5671 |
| 450737 | AW007152 | Hs63325 | transmembrane protease, serine 4 | 2.00 | 4157 |
| 433184 | AA147979 | Hs.285005 | mitochondrial import receptor Tom22 | 2.00 | 2746 |
| 427621 | BE621182 | Hs.179882 | hypothetical protein FLJ12443 | 2.00 | 2173 |

TABLE 12B

| Pkey | CAT number | Accession |
|---|---|---|
| 428342 | 6712_1 | AK056315 AI015524 AA724079 BI713619 AI377728 AW293682 AI928140 AI092404 AI085630 AA731340 BM469629 AW968804 AA425658 AA769094 BF446026 AW118719 |
| | | AI332765 AW500888 AW576556 AI859571 AW499664 AW614573 AW629495 AW505314 W74704 |
| | | AI356361 AI923640 AW070509 AI521500 AL042095 AA609309 AA761319 AI381489 H45700 AA761333 AW265424 AA909524 AA635311 |
| | | AA649040 AI392620 Z40708 AI985564 AW263513 AA913892 AI693486 AW263502 AI806164 AW291137 BI061872 BI059498 AA134476 |
| | | AW084888 AA036967 AW370823 T55263 BI002756 AA489664 BF827261 W74741 BF963166 |
| 412446 | 63467_1 | BC021735 AI669212 AL120184 AI769949 BE701002 BE184363 BE819031 BG702238 BF090049 BF963318 BF961912 BF943013 |
| | | AA934514 AA151245 BF960659 AA987907 Z41449 BF908059 BF908053 BF908049 BE699424 BF908060 BF962832 BF952020 BF963134 |
| | | BI035538 BF908052 BF908057 BF090026 BF943158 AI632924 BF512340 BF952021 BF960776 BF943437 BF942847 AI768015 F09778 |
| | | F04816 F02721 AA102645 AI633838 AA617929 BF947001 BI035448 BE935876 AW890837 AW898604 BF957405 BF963433 BG704815 |
| 447329 | 9170_1 | BC001573 AK074197 AW967044 AW360965 AW612048 AI765501 AA112034 BI859391 BI334670 W63553 AV706135 AW024203 BI194441 |
| | | AV655141 AA316041 BE280583 BG428780 BE267060 AV648926 AA317168 BI222294 BG437688 BE709273 AI951240 AI611162 |
| | | AW970792 H89338 BE543309 AL036038 R26941 BI765176 AA209487 AA111975 AA086463 AW192209 AW025245 BF511894 AW264490 |
| | | AW014985 AA947336 F15843 BI335083 AA563626 BE546579 AW470009 AA083693 BE090517 BF970294 BG427898 BE541527 |
| 451807 | 17758_2 | BM479185 AL552795 AL577722 BF038888 BM127617 BF510346 AW450652 AA865478 AW449519 BM127314 AI806539 AW449522 |
| | | AA993634 AI827626 AA904788 |
| 449349 | 852_3 | BG679689 AW856638 BM016319 BE327123 AW772128 BE693337 BE938262 BG013928 BF892774 BF894765 BF892588 BF890995 |
| | | BE155056 BE153569 AI934407 BE672538 AW204203 AA778306 BE502974 AI718504 AA778312 AW008224 AW299732 AI911561 |
| | | AI867457 AI521962 AI640173 AI823832 AI288888 BE467960 AI934441 AA483527 AW612103 AI802712 AW342106 AI580299 AW083293 |
| | | AI700874 AW469932 AI583726 AW302136 BE327360 AW614404 C02300 AA934834 F29737 AI262050 AA934619 AA535965 BF196507 |
| | | AA393480 BF086615 AI825386 AA009773 BI333272 T93614 AW770207 BF766665 T64641 W92713 R94110 T89897 BF086603 T93659 |
| | | AA001207 BE539257 BE541430 BE160783 BE155304 BE155454 BF891405 BF762818 |
| 429228 | 215430_1 | BG676155 BM009591 AI479075 AI025794 AI017967 AA448270 BE466812 AA853422 AI392649 BG952034 AA513384 BF840124 BE714620 |
| | | AW969605 AI553633 |
| 411605 | 10026_3 | BG256892 H10532 N46614 R52610 AW977696 BM460488 W56819 BI042183 BG977498 BE767451 BF870009 BG477472 R61137 R14274 |
| | | R20259 R09686 BI838226 BF034269 AA429173 BE741829 AW867495 AI123683 AW006831 BE831162 AW452753 AV742717 W86152 |
| | | BF115102 AI633815 BF921562 AA094230 BE092587 W86151 AA526153 AI672156 BF914496 R12579 BF852352 AA699780 T57386 |
| | | BF903022 R09933 AA678298 |
| 451752 | 10408_5 | AB032997 AI141678 AW978722 BE467119 AI761408 BF727385 AW237035 AI934521 BF436248 AI479668 Z40632 AA832081 AW295901 |
| | | BF057835 BE465977 AI621269 BE465983 BF756369 N74056 AI817896 AA716567 AA934774 H62600 H09497 BF943762 BE395335 |
| | | BE883333 |
| 444172 | 49300_2 | BF526827 AA513594 AL515291 AV648373 AV648176 AA916789 BF002906 AW469960 BE466943 AI367749 AI559715 BF431260 |
| | | AA937968 AI422252 AI288937 BF962778 AA909144 BF960004 BE671534 AI271719 BF925335 BE669504 BF433431 BF924838 AI218062 |
| | | AW960577 AV722716 BI859067 BF944964 BE147740 BF938993 |
| 400205 | 2538_1 | NM_006265 D38551 X98294 BM477931 BM461566 AU123557 AU133303 AU134649 AW500421 BM172439 AW500587 AW503665 |
| | | AW504355 AW503640 BM152454 AW505260 AI815984 AW504075 AW500716 AL597310 BC001229 BM474371 AA984202 AU135205 |
| | | BE090841 AW163750 BF747730 BF898637 AI206506 AV660870 AV692110 AW386830 AV656831 N84710 AW993470 BF086802 |
| | | BF758454 BG960772 BF757769 BI870853 BE018627 C75436 AW148744 BF757753 BG622067 BE909924 AA708208 BG530266 BF968015 |
| | | AW992930 BF888862 BG536628 AA143164 AW748953 BG498922 BF885190 BF889005 BF754781 BF800003 BM476529 AI627668 |
| | | AW028126 AL046011 BF590668 AI017447 AA579936 AI367597 AA699622 BE280597 AI124620 AI082548 AW274985 AA677870 AI056767 |
| | | BE551689 AA287642 H94499 AI752427 AI652365 AW002374 AW062651 AA360834 N68822 AU135442 AU125960 Z78334 BE545813 |
| | | AI092115 BF312771 BF242859 BG533616 BG533761 BG164745 BG492433 BM473183 AA172043 AA172069 AU157092 AU151353 |
| | | AU155318 BE302211 AI375022 AA085641 AU157923 H88858 AA132730 AA115113 AA909781 AI475256 AA424206 AW572383 AW084296 |
| | | AI184820 AI469178 AA782432 H92184 AA340562 BF195818 AA852821 AW576342 AA827107 AA173317 AW190014 AI918514 AA729372 |
| | | AA729718 AI055958 AA331424 BE328601 AA515690 BI018896 AW628277 AA748368 AA626222 BG492636 AW380620 BF800058 |
| | | AW370956 AA290909 R25857 BG952995 BF801437 AA172077 AU155890 AU149783 AI720904 AA902936 AA865727 AI470830 AV740677 |
| | | AA142982 AA482485 AU145485 AW576399 AU156042 R63448 BF246427 BE928472 D25910 BF758439 BF968785 BE565238 AA355981 AI905607 BG291148 BG533096 |
| | | BG532888 BF030886 BG613756 BE928471 BG574501 AA187596 AA361196 T95557 BG531446 |
| | | BG527242 BG527513 BG611106 AA085995 BF847252 BG024608 BE540261 BG531236 AL579993 BG108733 BG483503 BG571032 |
| | | BG492505 |
| 419631 | 2743_1 | BC022323 AF204171 NM_022361 BM264431 BE670789 AW188117 AI025298 AA861832 H84897 AI382294 AA662874 AW993380 |
| | | BE813742 H84368 AI188074 N20482 H84369 |
| 400277 | 170_1 | Y00281 NM_002950 BC010839 BC007995 BG675232 BM468552 AL555484 BG831516 BF035300 BG677277 BF852972 BE314901 |
| | | BF850656 AI371816 AA292474 AA375747 AA308414 BM454544 BI333370 BM049921 BI461428 BI465007 BI223401 BE856245 AW821164 |
| | | BF914775 BF914761 AU125835 BI222678 BI091137 BF340536 BM462798 BI224452 BG707915 AL569160 AA443815 AW572867 |
| | | AW363410 BF739268 BG010283 BI013120 BF818845 BF763468 AA305165 AI630370 AA039826 R24906 H02046 T96891 BF981330 |
| | | AW936510 AA478169 H04587 BG166574 BI869342 BE562482 BE539637 AA165089 AL579118 AL553699 BE044054 AW117440 AI520674 |
| | | BF435417 AW245648 AI952404 T29534 AU153459 AU152168 AW591591 AU146918 AI393187 AA478013 AU148143 AI224471 AI640728 |
| | | AI871537 AW264752 N93787 AI189357 AV756134 AI471659 AU147466 AA779206 AU149419 AU149104 AU159135 AA312221 AW445119 |
| | | AW021912 AI799771 F04407 AI285530 AI914643 AW068751 AA513325 AA164627 AA639285 AA569644 T96892 AI923594 BF439180 |
| | | BI770936 BF032438 AU154884 AA682793 AW072992 AU158815 AI884444 AL048031 AU158922 AU152546 AI695187 AL048033 AI245650 |
| | | AU148507 AW467451 BE536868 BF913001 BF062707 AL573082 AW067993 AA523354 BE886727 AI890705 AU159092 AI982693 |
| | | AI817553 AA236729 AI687858 BG163767 AI524675 AI678155 AA127100 AI762661 AU159718 AI469720 AA483627 AW131696 R26868 |
| | | AI199885 AW875614 AW938694 AW578974 BI763988 BG819168 BE874767 BG978292 BE162948 AL555483 AW189719 T56783 AI018819 AI476552 BI492837 |
| | | AI824440 BG996262 AA932887 AI380726 R79530 AA622108 AI262575 T56782 R27437 BE784153 AW129549 |
| | | AI675567 AI866759 BG987935 |
| 449444 | 2735_1 | U59185 NM_004696 AV734324 AI245349 AA369517 H88760 D79128 AA970406 H01059 H88761 H03446 BG620383 AU135008 AU136895 |
| | | AU158158 AU155762 R73608 R65751 R23756 N74630 AW078687 BE439761 BE786351 R68994 BE785867 AW297502 AW297553 |
| | | BG431545 AW814843 BF382644 BG429539 BE929862 BF811258 |
| 442961 | 60316_2 | BE966247 BE220885 BE467384 BE350135 BE672094 AI811582 AW665254 AA772731 AI283601 AA417067 AW197746 AI868357 |
| | | AI792143 AA931120 AI758506 AA843761 BE737582 AW379586 N38812 BG567321 H13257 |
| 443695 | 20416_12 | BE535598 AW204099 AW301249 AA609749 BF917914 AA775742 AV646137 AV646389 AA314747 |
| 422150 | 782_1 | BI086421 BF059136 AW003898 BF446659 AI632891 AI628067 AI703179 AI961149 BF111022 AW614154 BE674215 AA687350 AA779426 |
| | | BF591963 AW243344 AI356530 AI492508 AI694049 AI090422 AA465307 AI273387 BE674625 AW271971 AA969153 AW468593 AA984014 |
| | | AI817491 AA970258 AI914450 AI018697 AW577591 AW577616 AA382101 AW954455 AI867266 AI707995 AI337384 BF208406 BF037100 |
| | | BF223433 BF195517 BE673984 BF224124 BE813387 BG036579 BI553906 AA304995 AW361269 AW754160 AW361276 AW361271 |
| | | AI867118 AW805555 AW361284 AW954458 AW958551 BG681507 H79011 AW205696 AW134957 AA747667 AW753296 BF939060 |
| | | AW958549 BF910827 BG573750 BG168639 |
| 437834 | 294580_1 | BG110129 AW749287 BE535498 AW749299 AW749293 AW749302 AW749298 AW749291 AW749294 AW749289 AW749288 AW749296 |
| | | AA769294 AW749297 AW749295 AW749292 BE002573 |
| 439574 | 689966_1 | BG532820 AW246001 AI469788 AI350090 AI446788 BE549330 W84862 AA837988 |
| 433023 | 3970_8 | BE999967 BF438599 AW864793 AI802899 BE815132 AW468888 AI672189 AI052004 BF112024 AA772335 AW275054 AA573845 |
| | | AI144148 AI968683 AA846676 AA927355 H80424 AW973295 R88209 F29868 BE928871 |
| 407198 | | |
| 447349 | 1063443_1 | BE743847 AW809603 BM469626 AI375546 |
| 458098 | 23945_1 | AI082245 BE467534 AI797130 BE467063 BE467767 BE218421 AI694996 BE327781 BE327407 BE833829 AA989054 AA459718 BE833855 |
| | | BE550224 AA832519 AF086393 AV733386 BE465409 N29245 W07677 AA482971 BE503548 H18151 AA461301 W79223 W74510 |
| | | AI090689 AL600773 AL600781 N46003 R28075 R34182 BE071550 AW885857 AI276145 AI276696 H97808 N20540 AI468553 |
| 430935 | 15297_3 | BC017923 AA789302 AW466994 BF513878 AI819642 AI184913 AW469044 AI220572 AW072916 AI280239 AI473611 AW841126 D60937 |
| | | AA489195 N59350 AA693435 BG531204 AA484243 AW514092 |
| 412537 | 14066_1 | AK025201 AA425472 AI694282 BG057305 AA907787 AI286170 AI684577 AJ420494 AI809865 BF058095 AI478773 AI160445 AL044114 |
| | | AW665529 AI129239 AW297152 AI268215 AI469807 AI969353 BE552356 N66509 AA736741 AA382555 AW075811 AV759188 BI259364 |
| | | BF445142 BG232065 AI141758 AI631202 AI167566 AI208445 AA889823 BF982682 N90322 BI090882 BF208005 AW953918 AL044113 |
| | | AI016793 AA382556 AW235763 AA927051 AI862075 BE886691 BE619282 |
| 414770 | 41721_1 | AK056926 BG473673 BG482256 AL135566 AW419211 BF949370 AL120313 AV703730 H82569 BG012696 R27084 AA304583 BM452908 |
| | | BF516419 BF515687 BG036572 BG696740 AW953552 AW859437 AA306038 AI754064 AA608729 AA664163 H65119 AW272606 |
| | | BF942099 AW130468 BI089467 BG821499 AA152403 BF310450 BF314240 BG830310 BF803223 BG764269 BE542645 BE259142 R26953 |
| | | BE257224 BG475461 BF677821 R33048 |
| 438714 | 2576235_1 | AA814859 AI582623 AA814857 |
| 432339 | 57992_1 | BG823745 AW411259 BG111139 BM041741 AW015049 AW068953 AW014085 AA969360 AI027260 AI948416 AW015886 AA918278 |
| | | AA705292 AA702483 AA534205 BE856934 AI129462 BE926765 AI684275 R52686 N34869 H23555 |
| 438869 | 52134_1 | AF075009 R63109 R63068 |
| 431912 | 610_10 | BI763666 BI517886 BI759051 AI688604 AI660552 BF588523 AW004785 AW295479 BF591117 BF002672 BF064073 AA594441 AI380340 |
| | | AI700219 AI659950 AI688540 AW296326 |
| 450251 | 40382_4 | BE080483 AI689298 BE080416 |
| 412452 | 71091_1 | BE796667 BF330981 BE394193 Z45547 BG490525 F35734 AA130708 AA577072 AA446587 AA215665 AA978209 BG740729 BG746810 |
| | | BE298184 AI356291 AI671975 AI818924 AV715722 AI078381 BI142391 AI201085 AI198283 AI077572 AI694848 AW016425 BM456416 |
| | | AI277223 AW771476 F26140 AA102778 AW025780 R44726 AA761079 AI581346 AI991909 BM005939 BE537999 BG469717 AA114156 |
| | | BF437200 BE774942 |
| 418259 | 133853_1 | BM310925 AA426110 BM310629 BF434286 AW015091 BF475996 AW118867 BE675186 AI688568 AI453594 AW590589 AI652425 |
| | | AI827969 BF056946 AI802866 AI393380 AI476224 AW590639 AW136271 AI458252 AI524726 AA843768 AA782158 AI336058 AI097532 |
| | | AW451563 AA459408 AA459633 AA418444 W23607 BG940150 AI493445 AW054729 AI221929 AI868744 AA215405 AA766713 AA621546 |
| | | BF928317 BE464132 AI990909 AW271459 AI262061 AA215404 N74332 BG940151 BG952261 AA972115 W96315 AA689586 R69057 |
| | | BF766886 BE769254 W05240 |
| 452279 | 11990_1 | AF392454 AK023074 AI884890 AI814455 AW966220 BF736545 AA026021 AA286843 AA251918 BG197710 AA026294 AA337356 |
| | | BC010422 AK023226 NM_022776 BM459496 AA769310 AI826460 AU153650 AU160375 AW166211 AW292992 BF433538 AI823888 |
| | | AI684798 AI655985 AW770982 AI400454 AI276257 AA639510 AI689818 AW772604 AA807639 AU130298 AU132028 BF900889 BF904822 |
| | | AY007102 AU143256 BG621460 AA829630 AI864665 AI084922 AA025234 AI360060 AA766554 AA026295 AA825817 AA251762 |
| | | BE180751 BM464530 AV715833 AA779447 AI452519 AW418525 AA435643 D25894 AA435651 AA286844 N64369 AI702262 BG288063 BE170545 BM466232 BE299160 |
| | | AA169573 AL567428 BF217285 BE967276 |
| 449230 | 6182_6 | BC006329 AK026224 BG105365 AI472084 AW074277 AI743908 BM309990 BI850432 AI094365 AI539568 AW779999 AI261365 AA988975 |
| | | AI436272 AI559886 BI710742 AA988508 AA036678 AI867147 AW518513 AI620019 BE645777 AI801919 AW205320 AI457722 BF061996 |
| | | BF061798 AA233854 AA232795 AA516294 AA830561 R74220 BG768337 AW392972 BE764979 BE257665 |
| 413413 | 26025_3 | BG261240 BG231707 AI440201 BF540868 BF240794 BE779394 BG505096 AA129208 D82298 T10758 AU155599 BF857123 BF836348 |
| 446873 | 15731_1 | BC016767 Z30093 NM_001516 BI753904 AL555563 AL598882 BG706296 BG723901 AL600911 AW949486 AL602675 AL600169 |
| | | AL600111 AI373329 BF149149 AU099109 AL578036 AA410532 AA521208 BI018894 BI333338 AW269273 N79350 AW954400 AI760772 |
| | | AA765916 AW300373 AW628851 AW505118 AA506136 AW779373 AI675026 BE674159 AI241579 N51870 AI632936 AI796901 AI620962 |
| | | AA876979 AA886127 AA934448 H58689 AA373345 N78420 AA282406 N98953 N22575 N62381 AA805377 AV718946 AI421009 D20327 |
| | | AW518433 AI554439 BI027833 BG613441 BG573433 N66669 N62701 |
| 446999 | 70312_1 | AA151520 AI749635 AA149436 BE172702 AW317084 AA922501 AI302818 AI147563 AA789216 AI719336 AW612978 F34536 AI971386 |
| | | AI246525 AI183312 R02554 AI360172 AA634282 AI022935 AA639461 AI086411 AI087086 AA633082 AI590029 AA856582 AW369734 |
| | | AA150042 AA877171 AA456459 AI078529 R83333 AI161298 AA056987 AI350120 H88127 AA258759 AI673598 AA454566 AI342790 |
| | | AI492606 AI159945 AI198009 AI198039 AI142751 AI141403 T81478 AW014110 AI197808 AI927796 AA534936 AA649735 AA649697 |
| | | AI349452 AA719660 AW954285 |
| 458997 | 11847_4 | BM453041 AA760783 BE218582 AI340046 AW166131 BF515854 AI630296 AA461307 AI090881 AW023059 AA155797 AA115486 |
| | | AL597396 AW889004 AW937420 AA137082 AA013374 BG619478 BG401839 |
| 455019 | 186896_1 | BG339523 BG000719 BF913518 BG703361 AW851100 AW850833 BF802227 AW850818 BI088683 |
| 441128 | 20932_1 | BC014072 BE328850 AI356567 AI148171 AI022165 BG149661 BF000671 AA233101 AA573721 AA447991 AW016855 AI005068 AA554071 |
| | | BF478215 AA906902 AW014761 BE905651 BE512923 BM047129 AA243852 AA232991 AA127550 AA127551 AA570256 AI473237 |
| | | BF033706 N90525 AW973623 AI359627 BG674574 BE903322 |
| 455459 | 1174365_1 | AW947654 AW947665 AW947633 |
| 437866 | 34267_1 | U52054 AL581000 AA156850 AW293839 BI335865 AA024963 BF149420 BE073977 AW602574 BE164012 BE163992 BE163974 |
| | | AW402161 BM194134 AW966609 W84374 BF916380 AA385173 W84366 AA383743 BF903598 AA043776 W84421 AA778446 AW444904 |
| | | BF446960 AA837481 AV755539 AW468444 AW468002 AA811830 AA581806 AI866686 AI572124 AA687333 D20160 AA812489 AU185248 |
| | | AU186004 AA156781 AI536733 BM144850 AI471883 AA040926 BF507639 AA043777 AW874142 BE832523 BE163972 BI022546 BI021204 |
| 443687 | 1347_10 | H72674 BG925294 AA699932 BF061529 AI240821 AA676904 AI083582 AA766036 AA761066 AA761228 AV686983 AV646138 AV646121 |
| | | AW961968 AA004250 AV658573 T08315 AA004669 |
| 437204 | 28243_1 | AL110216 AI816283 AI688476 AA928351 AW157672 AI288740 BE468242 AI452476 AI291665 AW192364 BE503635 AW592672 AA857393 |
| | | AW162770 AI680660 AI879565 AI347717 BF880800 AI266646 AA834381 AI291667 AI264567 Z38449 T03352 AI929432 BE673697 |
| | | AI124770 AI350389 AA910378 R54042 AV746670 AW089037 AV729218 H28971 R38549 AI692560 BE671562 BE669916 BE672206 |
| | | BF445152 BF445151 AI914323 AI684406 AA932348 BE670597 AA932080 |
| 412314 | 17239_1 | NM_018971 AB040799 BF060724 BF061085 BF058335 BF446952 BE502582 AW051846 AW468602 BG507049 F12518 BE703222 T19393 |
| | | T32177 T74004 AA775876 BG370235 AA426547 AI857339 H08634 AW961071 AI703476 AW419310 AW118126 BF055108 AA931093 |
| | | AI298553 AI343013 AI168160 AA976583 AA622237 BE327125 W51947 AI056138 AA825247 AI348336 AA101674 BF222266 BE173228 |
| | | BG924257 F10138 AA757564 H08635 |
| 453160 | 6028_5 | BC009612 NM_003526 BI597616 AV761592 AV760377 AL601008 BI604131 BE645918 BG187760 BG181525 BG210634 BG192999 |
| | | AI263307 AA344186 AW952966 AA033609 AA037562 AA722183 R79452 H70775 BF674991 BE769437 BG007856 AA037483 AW572535 |
| | | AI143991 AA084581 AA033610 AV742510 AV735788 R08336 |
| 438118 | 1007899_1 | AA778411 AW663081 AW753311 |
| 430712 | 301999_1 | AW044647 AI670953 AI656180 AA484715 AI659205 BF923472 |
| 406685 | 0_0 | M18728 |
| 411261 | 1074276_1 | AW834670 AW834476 AW834691 AW834604 AW834655 AW834623 |
| 438697 | 1239615_1 | AW975849 AA814439 AI052090 |
| 449343 | 14470_1 | AK055378 BE896063 AV722833 BI032095 BF952019 BG547204 AI151418 W03542 W60401 AI346936 AA862855 W60310 N72501 |
| | | BF963436 C04881 H90060 AA001203 AI863984 AI932612 AI499771 AI187944 AA501896 AA714924 H00689 AA918680 AA573996 |
| | | AA521308 AW182868 BF996707 AA908959 AI628880 AW173363 AW665845 AA130178 AI818267 AI653663 AI828924 AA746655 AI951984 |
| | | AI635625 AI093113 AI377976 AI624029 AI418242 R76291 W92652 AI207798 AV706224 AA742467 AA641806 W61229 AA130170 |
| | | AA160170 H85007 W72474 W61163 H97873 AL047509 R76567 AA812071 H81599 AA021275 H85004 H85894 BG537537 BF830518 W76228 W46673 Z43839 R78710 C01747 |
| | | H00789 BI036345 W92828 BE150445 AW380821 AW173095 H85630 H81598 H86032 R84855 R13223 AA774992 |
| 424036 | 6226_1 | NM_033445 BC001193 AI885781 BF794032 AA476620 AA810906 AA810905 AI291244 AI885097 AI359708 AI335629 H97396 AI344589 |
| | | AA300377 AA457566 AW771833 BE465621 AI364068 AI364452 AI648505 AI918342 AI928670 AA886580 AL531029 AA886344 AI186419 |
| | | BG329096 BM045465 AL531028 BG437151 BE868021 AA179427 |
| 452598 | 2741_1 | AK055715 AI129800 AW027959 AI927949 AI650270 AW514661 AI708393 AL138076 AI926721 AA749139 AI399955 AW874011 AI302816 |
| | | AI242763 AA828301 AA262795 AI093249 AI084565 AA904444 BE245661 AI810709 N30447 AI653617 AI289975 AI474418 AA737455 |
| | | AI619613 AA291708 AI640264 N91446 AW029069 AA680378 AA338773 BC022321 BM450359 BE439944 AA521348 AI066761 AL138075 |
| | | BM153113 BM153112 AW182953 BM147809 AI247395 AI911116 AA977587 AA291690 AI243777 F35824 AA579199 BM479711 AW961031 |
| | | H92980 H86861 AA026904 AI052445 R92739 T10789 AV749986 AL540531 AA452953 BM152599 AA382769 BI550695 BG432646 |
| | | AV744808 |
| 441420 | 2361943_1 | W28068 T96110 AA932872 W28643 |
| 447832 | 1036795_1 | AI628543 AI433357 AW772732 |
| 440282 | 21425_1 | NM_021188 U90919 BM458077 BG214941 AL562127 BG197712 AW994207 AA043114 AW993474 AA631832 AI911529 BI001014 |
| | | BE156322 BG319454 H27376 BG220618 BG189474 BG325006 AL519085 BM472894 AL520560 BG575806 AA319317 BG621802 |
| | | BG912722 AV760869 AV760871 R13243 BE274097 BG327062 BI765172 BE614338 BG214923 BM153391 AU147675 N58119 AL043557 |
| | | AL043558 AL043510 AA773474 AU148079 BF062869 AW189972 AW250398 AI028605 AI434688 AW149344 AI246783 AI961376 AI698209 |
| | | AA251535 AU160747 AA579811 AI572570 AI656228 AA426093 AA127171 BF222487 AA725576 F21161 AI932217 BF222251 AI024660 |
| | | AA780998 AI538862 AW140038 AI122909 AA493515 AA401920 Z39789 F10942 F05081 AA814868 F10376 AA776314 AA993104 T94991 |
| | | AW583990 AA128211 F03074 AL579261 AL519084 AA384895 BG196670 AW083526 BF220181 BE669988 BF907279 T23499 AL520559 |
| | | AU149005 N38766 H96506 AA040865 AI263737 AA723350 AI168145 AA652386 AI374699 AA487377 AI927374 AI985531 AI352105 |
| | | AI040092 AA486562 BM053020 Z41738 BE243710 AA601558 BM052743 AI445827 AA487260 AI982679 AW130546 AI743384 AI335647 |
| | | AW978172 R40324 AV736436 BE966360 AI185467 AI367193 N75025 AW131068 BE940519 H43910 AA486659 BE614365 BF220261 |
| | | BE181357 BG189473 BG210176 H43958 |
| 434804 | 118455_1 | AA659316 H64973 AA649530 |
| 427521 | 513212_1 | AW973352 BF222929 AW016853 BF059130 AI651829 BE551767 AA558414 AI339359 BF059601 AI961162 AI341422 AI206248 AI206165 |
| | | AA548736 AA768578 AI539081 AW025957 AA736837 N79575 AW594357 AA480892 |
| 455416 | 1164250_1 | AW937150 AW937151 AW937141 AW937174 AW937132 AW937173 AW937170 AW937165 AW937195 AW937145 AW937164 AW937137 |
| | | AW937160 AW937142 AW937171 AW937191 AW937139 AW937143 AW937159 AW937140 AW937163 AW937135 AW937179 AW937156 |
| 453454 | 2736_1 | BC001588 BC007424 AF016369 NM_004697 BI756186 BI257019 BG500792 BI862776 AL121371 BG574833 AA703250 AA179511 |
| | | AW052006 AI280150 AI914000 AI358319 AI081204 AI082594 AA992449 AI470821 AI655744 AW237529 AA678858 AI984430 BF433055 |
| | | BE467594 BE467573 AA035630 AI289987 AI184802 AI681391 AW592416 AI138377 AI139266 AA961714 AI800163 AA418751 AW451928 |
| | | AA668676 AI273444 AI494387 BE046912 AI276555 BF196021 AA700055 AA609305 AA772596 AI635758 AI635749 H95459 AW610290 |
| | | BE464994 AA527136 BF374802 AI800175 AW195227 AI189676 BF802049 AL513632 AL554911 AL538845 BE297273 AA315321 |
| | | BM451920 BE269268 BE292835 BE018128 BG755713 BM041095 BG677009 AL039691 BF995709 BE735586 BE296453 BG393609 |
| | | BG824453 AL567522 AI745257 AW388641 AW301265 AI141144 AW029280 AU149362 AU152328 AA418960 AL121009 AI890398 AL528748 H13050 T47086 BI000575 |
| | | BF334914 BF109661 R44450 H13259 T47087 AW388646 BF305834 AL577515 BM041600 BE889299 |
| | | BF239768 |
| 438989 | 1242606_1 | AW979100 AA829131 AA828926 |
| 454731 | 1069368_1 | BE067650 AW817053 BI000539 |
| 455751 | 1497052_1 | BE075281 BE075119 BE075123 BE075219 BE075046 |
| 408673 | 13031_1 | BG027435 BF746745 AF349017 NM_052963 AL553524 AL519055 AL525199 AL529016 BE208517 BI856042 BG480978 BE267120 |
| | | AW815679 AL525198 AL561760 AA933030 AA634332 AA568280 AA927487 AW182340 AW592604 AW732157 AI680072 AI431328 |
| | | AI357601 AI872335 AI032112 D25780 AL529015 AL576497 AA575842 BG223081 AA618007 BF310845 BF757605 BM012536 BI911537 |
| | | BE964237 |
| 440546 | 631538_2 | AA889258 AW139809 AI700895 AI491994 |
| 434438 | 111581_2 | AI654867 AW236852 AW236558 AA747964 BE843851 |
| 445138 | 2047826_1 | H97479 AI214273 H99085 |
| 412122 | 3941_2 | AK056423 AW070901 BG222419 BF928848 AL567752 AW770702 BE160226 AW248894 AI304851 AI090802 BF514704 AI659696 |
| | | AI762084 BE018718 AI088023 BF876482 AA283824 BF742200 AI349494 AI942345 AL576641 BG959608 AA853964 AA854927 AA767839 |
| | | AI955028 BG611080 BI497416 AW947186 AI703492 AW612137 AI696372 BM016022 AW852632 N57282 BI859575 AA100379 AI879394 |
| | | AI653605 BM459051 AI446574 BG031916 BI004602 BM014817 AA725075 BE063022 BG741166 BF088949 AI937125 AW936878 |
| 437158 | 59575_1 | AL050068 AA160485 AW173544 AW296506 AW439860 AI521563 AI702529 AI393606 AW138323 AA570109 H19504 BM021968 BF063327 |
| | | BF593552 AA630766 AI597717 AI807128 AA523012 AI356250 AW451857 AA974203 AI762577 BF512552 AW007307 BE675286 |
| | | AW450602 AA962057 AW516069 AI582546 BF221924 BF222543 AI801808 AW468599 AW000736 AI866625 AW235356 BM021837 |
| | | AA911956 AI680606 W86516 T03370 AW611634 H41653 AI468349 H19588 AW090198 AW043993 R39847 |
| 442092 | 22756_2 | BM449821 BM069895 BM023170 AW025563 BG152606 BM023452 AI862106 BG959957 |
| 450687 | 82562_1 | AA010736 AA495800 AA640726 AA654716 AA495737 |
| 441318 | 1989216_1 | AA928233 AI078234 AI702558 H81727 |
| 433553 | 11940_1 | AL583707 BM013793 AA765477 AK025032 AL582648 BI761445 AW977217 AA700263 H65650 AA309842 BM020355 BM449517 AI752899 |
| | | BE677851 AL121362 AI955600 F11510 BI861721 AI022695 BF154943 BE081847 BM046024 BE883957 BG109038 BF593817 AA825523 |
| | | AA744888 AA825246 AA973546 AA599107 AW173711 BE550926 R40586 |
| 410507 | 4450_2 | AK027433 AF117064 NM_013319 BC004468 AI149901 AI150093 AI374696 AI566580 AA779898 BG696067 BG828923 BM051241 |
| | | BM050350 AL580560 AL558826 BG182261 BG194259 BG194258 BF036155 AI026803 AI024570 AA702281 AI566953 AW662600 |
| | | AA463546 F33147 AA357796 BE312357 AL516788 AW958856 BE730432 H85868 AA046292 BG478025 BG112231 BG763623 AA098922 |
| | | BI093481 BE746381 AW962126 AI040821 BG026983 AA355288 BE392859 AA085571 BF875433 BF316280 BG740116 BG166624 |
| 414388 | 750506_−2 | BE281412 |
| 433090 | 7504_2 | AB038318 BC008888 BE905346 BE301941 AA705936 AW014954 BE378742 AI720050 BE395327 BG951204 |
| 400183 | 48154_3 | X80199 NM_007359 BI553784 BG720095 BG899766 AI088926 BE857470 AI199713 AI870291 AL121179 AL577898 AA009896 AA868181 |
| | | AA482924 AI637524 AW043834 AU156777 T30547 R05481 BE902481 AW372677 W73547 H51965 BF944628 BE825156 AL567238 |
| | | AW372714 BF759809 AL578496 BE396176 H02384 BE007647 BF917244 |
| 455286 | 1149378_1 | W27935 AW887403 AW887474 BE144384 BE144386 |
| 451378 | 1524483_1 | BE153447 BE153429 |
| 455440 | 1172091_1 | AW946022 AW946090 AW946040 AW945962 AW945979 AW945971 AW945960 AW945956 AW946031 |
| 412560 | 72553_1 | BF002870 AI003925 AI082639 AA194383 AA702993 AI767866 AL575096 BF593252 AI948584 AI678666 BI963722 AI765219 AA620965 |
| | | BE671938 AI004689 AI004690 AI990303 AI127228 BE856290 AW203978 AI934786 AI770075 AI144132 AA812597 AI813349 AI142908 |
| | | BE671242 AI208243 H82735 BF115200 AJ345984 BE044308 F32992 AJ346047 F24958 AJ346565 AJ346456 F18071 R24502 BI830577 |
| | | BI222716 H83611 AA507760 BE463806 AA194467 AI865963 BF434962 C04894 AA813511 AA112966 N89963 |
| 456564 | 5823_1 | AW369406 AW747802 AW991351 AW747815 |
| 417879 | 2158126_1 | BF061412 R22451 R22128 R33566 |
| 434698 | 116401_1 | BE044674 AA643497 AA643491 BF935026 BF746059 BF908549 BF907921 BF908551 BF908547 BF907923 BF907919 BE044737 |
| | | BI017933 BG952887 BI017769 BI017771 BI017932 BI017767 |
| 418304 | 1093209_1 | BE883520 BI057842 AA215702 AA215703 AA368006 BE006876 BE066555 |
| 452480 | 3144986_1 | AI903574 AI903572 AI903526 AI903571 |
| 442643 | 2736_1 | BC001588 BC007424 AF016369 NM_004697 BI756186 BE257019 BG500792 BI862776 AL121371 BG574833 AA703250 AA179511 |
| | | AW052006 AI280150 AI914000 AI358319 AI081204 AI082594 AA992449 AI470821 AI655744 AW237529 AA678858 AI984430 BF433055 |
| | | BE467594 BE467573 AA035630 AI289987 AI184802 AI681391 AW592416 AI138377 AI139266 AA961714 AI800163 AA418751 AW451928 |
| | | AA668676 AI273444 AI494387 BE046912 AI276555 BF196021 AA700055 AA609305 AA772596 AI635758 AI635749 H95459 AW610290 |
| | | BE464994 AA527136 BF374802 AI800175 AW195227 AI189676 BF802049 AL513632 AL554911 AL538845 BE297273 AA315321 |
| | | BM451920 BE269268 BE292835 BE018128 BG755713 BM041095 BG677009 AL039691 BF995709 BE735586 BE296453 BG393609 |
| | | BG824453 AL567522 AI745257 AW388641 AW301265 AI141144 AW029280 AU149362 AU152328 AA418960 AL121009 AI890398 AL528748 H13050 T47086 BI000575 |
| | | BF334914 BF109661 R44450 H13259 T47087 AW388646 BF305834 AL577515 BM041600 BE889299 BF239768 |
| 459010 | 84453_1 | AW955786 BF870627 AA251104 AA331438 AA016068 Z19751 |
| 445008 | 781781_−1 | AI204903 |
| 416612 | 1980152_1 | N77403 H70565 H67949 |
| 416946 | 6767_3 | NM_016431 AF218778 AA322904 AA323084 AA323870 AA323104 BE263071 BE703587 AA351451 BE621564 AI377280 R22305 |
| | | AA974560 AA507924 AA972825 T16799 AA932063 AA722149 N53101 |
| 429978 | 35194_2 | BE738425 BE738323 BM126944 AW629678 AW265195 AI916735 AI394255 AI573090 AI354442 AW612857 AI339558 AI919424 AI377532 |
| | | AI354441 AI308821 AA772275 AW055215 AI589705 AI336532 AA806547 AV682125 H93575 AW071172 AW769904 AI863985 AW265018 |
| | | AW196655 D79662 BE042393 N75017 AW014741 C75509 BE748621 H92431 AW079261 AW901780 AA329482 AW960115 BI260621 |
| | | AI767525 R31663 BI918664 AW963196 C06195 AI678018 |
| 422259 | 140437_1 | BF821471 AW795791 BF844843 BF821371 AA307584 AW795790 BF833724 BE154067 BE064709 |
| 455236 | 1141653_1 | AW876000 AW875983 AW875972 AW875974 AW876050 AW875966 |
| 416128 | 3761_6 | AK057803 BG944795 AW411505 AW949210 BI018336 AI366964 BE165417 AA173988 BF965882 AA581362 AI002701 AA340708 |
| | | BF762925 BE005389 |
| 419544 | 236329_1 | BE787623 AI909153 AW362351 AW362269 AW362245 AA526337 AA244193 BF876842 R79395 AI909154 |
| 437147 | 3159_1 | AL049964 AW271733 AI417637 BF477833 AA890056 AA987650 AW269859 AW467559 AW952590 AW952591 BF197902 AI700840 |
| | | AW022147 AA335567 BM129024 AA336182 AA760676 R10321 AW450329 AI027902 AI536762 AV682039 R82056 N90743 BM068840 |
| | | T72673 AI224593 AI627551 H57901 R72123 R82002 AA180435 |
| 442562 | 39593_1 | AK056685 BG399272 AA187835 BF821903 AV660550 AV660556 AV660502 BG564397 BE379584 BF446961 AI653056 AW973709 |
| | | AI653173 BG054997 AI266043 BI054879 AI656750 AI492830 AW021142 AI472184 AW170056 AI082443 AI167921 D59940 BI492088 |
| | | H74180 AW130886 AI348677 AI278577 AA761517 AI698203 AA115535 AI264790 AW205074 AA860452 AA554902 AI000715 D62102 |
| | | BE544768 AI376090 D59939 AW242249 AA525421 R34211 R34328 BF248064 BF241437 BF572759 BF218832 |
| 437341 | 2493968_1 | AA809353 AA749062 AA825757 |
| 454453 | 8582_4 | BF313069 BE879305 AW752781 AW752727 AW752559 AW752578 AW752584 BF846118 AL545903 BF846115 AL525361 |
| 442476 | 2694863_1 | AF069477 AF069476 AF069475 |
| 451958 | 71947_1 | AK054586 AL526339 BG257506 AW303347 AA594114 AA770237 AW117421 AA992677 AI028634 AA947172 AI242148 AA641234 |
| | | AL574323 AI219861 AI806256 AI033441 AA532785 AA905552 BE908245 N56134 H28597 R00255 AA969941 AA515532 AA887328 |
| | | AW104956 AI825261 AW207328 BE552249 AL562032 BI459483 BF447646 AW341155 |
| 441181 | 31840_3 | AK027315 NM_032472 BG195928 BG940337 AA742244 BG195277 AA424400 AA812029 BG220715 AA416925 AA724544 BG755237 |
| | | BG489761 BG340645 BE696471 |
| 415889 | 12922_1 | NM_052918 AF284756 BE019093 Z42986 BE328250 BE207835 R54516 R24563 H08127 BI522616 AA551620 H07879 AI174481 BF941262 |
| | | BF222810 R54417 AW137766 AI638502 N22373 H08128 R44366 AW272405 AI675836 Z38786 N75618 F02463 AI654047 BI492031 |
| | | AW021081 |
| 441102 | 25369_9 | AK054859 AA973905 AI299888 H63235 AA917019 AV722171 BE514107 BM051585 BE513744 BE295938 W32901 AA649212 |
| 421342 | 267584_1 | AA504749 AA287498 |
| 441057 | 1565090_1 | AI187760 AA917993 AA913608 |
| 412988 | 1342150_1 | BE046680 BE046738 BE044958 |
| 421582 | 13358_1 | X00474 NM_003225 X52003 M12075 BI765761 AW950155 AI571948 BI760569 AA308400 AA568312 BI761955 AA507595 AA614579 |
| | | AA614409 BF747698 BM142326 AA307578 AI925552 AA578674 AA582084 AW009769 AA514776 AA588034 BG271505 AA858276 |
| | | BM142503 AW050700 AI307407 AI202532 AA524242 AI909772 AI970839 BG236516 AW750216 AA587613 AI909749 AI909751 AI910083 |
| | | AA614539 R55292 AA507418 |
| 400240 | 27222_1 | U04209 NM_005926 AU121948 BI089933 BG425282 BG686931 AW821182 AU100079 BF700972 AL565099 BG116731 BG200436 |
| | | AL555789 AL047536 BF197395 BE504960 AI304957 AA932419 AW575886 AI916292 BI033772 BG995988 AL570315 BE348695 AA331214 |
| | | AL514367 AA436797 AU134683 AL533163 BE818949 BE818946 BE818920 AU136916 AL047535 BG424079 BE294696 AU135607 |
| | | BE207326 AL514368 AA318042 BF831066 BG106341 AW865170 BE937045 AI828674 BM083453 BE385535 AW865372 BF350485 |
| | | AW382531 R01323 AA092464 |
| 432093 | 1237011_1 | AW972670 AA525808 H28359 H28383 |
| 451311 | 145689_1 | AW862765 AI769797 BF366694 BF734848 D61040 |
| 455290 | 1151240_1 | U75810 AW890252 |
| 416800 | 1995904_1 | W80508 W78708 H89652 |
| 409775 | 912104_1 | AW499605 AW501372 AW503065 AW500923 AW501639 |
| 410703 | 351960_1 | AW998552 AW998523 AW797867 BE171712 AW881832 AW798089 BF985045 AW998520 AW998553 AW797821 AW797971 AW998601 |
| 442604 | 3440_15 | BE790716 BE263710 BI019623 BI019157 AI003371 AW747962 AW747976 BF921877 |
| 424686 | 1228447_1 | AW963243 AA345251 AA345504 |
| 449570 | 79253_1 | AA001793 AA001871 |
| 455737 | 1494976_1 | BE072229 BE072246 BE072210 BE072225 BE072256 BE072221 BE072211 BE072242 |
| 418166 | 18858_1 | AK055915 BE867252 AI523348 AA765350 BF446858 Z43675 R19529 AL133837 BG389444 AW382942 BE702956 AA081961 BE835247 |
| | | BE835308 BE835295 AA376302 BE645790 AA375690 AA376592 AW954423 N85732 AA249770 AI754416 AA213816 BF592044 AA811729 |
| | | AW514842 AI633486 AI096810 AW183016 AI635738 N27524 BE645916 AI247842 AW991230 AI754277 H16814 AI766892 BF108422 |
| | | AI800361 T95129 F11313 R97946 R93989 AA375242 BF109388 AI860939 AI680060 AW953899 BF971486 AI972337 AW953841 |
| | | AA372437 AI216746 H11384 R38484 AA249043 AA249732 |
| 424339 | 50559_1 | NM_015720 AF219137 AL534420 AL524055 AL537346 AL538442 BG765888 AL530054 AL525377 BG474596 BG473144 BE251553 |
| | | BG706099 AL538039 BG703131 BE255806 BF805256 F12128 AL566773 BI828686 BF761480 AI204971 BG818818 BI199246 AL534816 |
| | | BF529941 AA324163 AL523285 BG914330 H07952 AL534815 BE769903 AI867802 BM310135 AL533702 BE254484 BF528852 BE867462 |
| | | BE740130 AL134164 AL567115 AL533701 AL524054 AL515904 AL523284 AL568203 AL534419 BF981162 BE257148 AL561833 |
| 441616 | 25638_3 | BG164527 BI757270 BM453023 BI759791 BG700379 AI300093 W80481 BE856240 AW263496 AA781414 AA922117 AI479737 AI499701 |
| | | AA742324 AW274584 AA885442 AI393574 AI926512 AA972701 AI685317 AI305153 AA434451 AI086693 AI356800 AI200095 AW627592 |
| | | AW613806 AI280399 AA837589 BE710524 AI334386 W47269 AI491703 AW338258 BG220604 AI422897 AI282124 AI218584 AI347082 AA731678 AI273274 AW022998 |
| | | AA256494 H84116 AI698561 AW182912 AI280442 AA480586 BG187364 BG216534 BG213821 BG198052 |
| | | BG191538 BG212262 AI381988 BF197074 C01000 BE621682 AV754612 AA365458 AW817587 |
| 412756 | 474357_1 | BM455759 BE698411 BE698282 BG107419 AA729879 W77904 N27422 AA210943 BE833271 |
| 445326 | 1883425_1 | AI452799 AI220072 F28010 |
| 448663 | 16112_4 | BE966763 AA659765 AI961656 AI520918 AA761743 AA281477 N66431 BE463652 AA281329 AW272944 AA058687 BM145087 AA045516 |
| | | AW341820 AA112515 AA258766 AI886639 AA714133 AA768245 AA035533 AI630459 T20165 AW971268 BE966269 AA522722 |
| 446937 | 8722_3 | AW731689 BG107453 BG178894 AV661328 AL602899 AA977190 AI189509 AW303818 AW207677 AI378518 AA581619 H99430 N30587 |
| | | BE219726 AI689955 AA463686 AI500120 AA878606 AW675161 AA463760 AW770776 |
| 423357 | 598107_1 | AI857318 AI285124 AI364333 AA324896 AW104203 |
| 407590 | 78212_1 | BE293321 AA631602 AA281564 AA927393 AI831258 AA340072 BG009209 AW956385 AW838390 N94346 BF724763 AW082728 |
| | | BE044419 AI417373 W69444 AI147614 AA632403 AI937606 AI864878 AI969211 AI081135 AA885000 AA926984 AA642586 AW079405 |
| | | AA973001 AW628858 AI521070 BG912959 BI052498 |
| 429588 | 1396089_1 | AI092511 AI080271 AA455177 AI381859 AI381721 |
| 411136 | 359870_1 | AW819430 AW819440 AW819425 AW819423 |
| 426901 | 985848_1 | AA827922 AA772123 AI827741 AI081071 AI393618 AI520759 AI393608 AA398670 |
| 445075 | 2823108_1 | AI651827 AI206885 AI699543 |
| 457370 | 1235569_1 | AW971004 AW970998 AA574217 AA493538 |
| 408839 | 234669_1 | AI355222 BF378422 AA366587 BF874552 AW277084 R26970 D79194 R27662 |
| 437810 | 6139_1 | NM_016617 AF208844 Z70222 AL515797 BI666750 BI602334 BG717532 AL554429 BI545991 AW847749 BG393649 AV757080 AI373461 |
| | | AA315780 AA301659 AI393910 AI762600 AA321522 AA331540 AF305827 BG924826 AI720715 AI079860 AA677539 AA707143 AW196875 |
| | | AI269926 AI929790 BE246362 AI753224 AI687566 AI022486 AA101081 BM150935 AI591063 AI566293 AW189841 AW515306 AW028380 |
| | | AA766135 AI084005 W02557 AI018470 AA972561 AI371710 AA954779 AA746269 AA747909 AW020207 BI491245 H89948 AI041765 |
| | | AV652999 AA889510 AW243602 AA628783 AI866249 AA736845 AL515796 BF970987 AW604215 AW604214 R53127 AA937309 |
| | | AA815183 AA976070 AA736764 AW473513 BF591731 H88829 AL566858 BI088846 R73732 BF679743 BG113547 BF243416 |
| 400272 | 863_2 | AF317549 X78926 BM457918 AW963214 BG568218 BG679871 BG912907 AV714355 AA081606 AI188398 BF792205 BF671399 |
| 410099 | 16732_1 | AK055674 AW965247 AV751598 AA290926 R53043 AA331387 AK056148 BI917678 BG819395 BG911971 BG820167 AI174254 AA348720 |
| | | AA364503 BG714279 AW893230 AA081774 H24222 AV727176 BF875715 AA081630 BE000834 AA334880 AL563737 BG029709 W52882 |
| | | AI439658 BE551237 AA283724 BF109530 AI457096 AI805992 BE467736 AA693467 AI697593 AI887863 AI167419 AW901980 AW901768 |
| | | BE702179 AA484549 T23811 BE327043 AA716027 AA917004 AA167714 BF339675 AA084618 AI418634 T31586 AA436630 AI366472 |
| | | AA706191 AI422304 AI204899 AI041169 AA211402 AW827081 AA788593 T32736 AI767935 AA747914 T03534 AW959843 AL119527 BE327037 AW901982 AW993370 |
| | | AW901977 AW902071 W60090 N79906 D52685 T07735 BE702069 BE702172 T08671 BE767121 BE767117 BE767113 |
| 451653 | MH115364_1 | BG119563 AW975776 BG498478 BE245304 AW450934 BF508792 AA599489 AA599477 AI805268 AA055489 AI128758 AA761425 |
| | | AA731858 AI302271 BE219432 AA687294 AA018972 AW629429 Z45675 AW961626 T30940 R34554 T84712 BG986694 |
| 407843 | 240894_1 | AW085847 BE155229 BE155223 BE065083 BE154014 BE153966 BF330765 BE155423 BE160795 BE155307 BF882890 BF350689 |
| 438578 | 2573130_1 | AA811244 AI373188 AI240509 |
| 400247 | 2764_1 | BC022339 BC009610 BC010537 X79805 NM_006713 U12979 BM467814 BM450743 AU132951 AU137129 BG493425 AV758819 |
| | | BG708412 BG705885 BG702217 AV716638 BG777009 BI545689 BI552153 BM476712 BG770858 BG527656 BG528277 BG391388 |
| | | AV716861 BI602926 BG290073 BI667399 BM451469 BI667173 BI602139 BG532171 BI669216 BI544727 BG721852 AV716503 AV701327 |
| | | BM090738 BI492000 AI308856 BI544904 AL599813 AV715829 AV716505 AV714587 AV717902 BF668072 AV716385 BI461927 BM090954 |
| | | AV717826 BG503676 AV647719 BG501392 BG428433 BE895629 BM313117 AW021050 BG435032 BM152910 AA313503 AA872377 |
| | | BG574714 AV712054 AV732696 AA252476 AV712759 AL599643 BE790872 BG654930 W73337 AW675377 AV760376 AV725139 |
| | | AV716379 AA887165 BE830003 AW023796 AL599291 AI902948 BG944042 F00781 AA352483 BG217897 N33888 AW581924 BG654730 |
| | | D31410 AA353088 D31288 AA295029 H95170 BE935104 AU139980 BG772963 BG776470 BG532512 BG105449 BI545421 AV715456 |
| | | AW386083 BG699714 AL535832 AL514940 BG190861 BG210593 AW999254 H95138 AA353863 BE764809 N50375 BE091363 BG701255 |
| | | BI860846 BI832485 BG168150 BG028647 BE546301 BG900321 BI909737 BG702363 BG614141 BG611137 BG700121 BF031492 N85802 |
| | | AV715940 N51590 BG993478 BE172016 AW893622 |
| 432586 | 6633_1 | BC022881 AU150944 BG750783 AW754175 AW857737 AI911659 AI050036 AA554053 AI826259 AA568548 |
| 446777 | 630089_1 | AI632332 AI341129 AW269288 AI825342 AW138243 |
| 427673 | 1371317_1 | AI989709 N98298 AI371995 AA412172 AA410427 |
| 437576 | 16775_3 | X97303 BM477862 AL555571 BI916746 BG777391 BE514383 BE312102 BM352234 BI439158 AW239231 BG535338 BE072000 BE071960 |
| | | AW577360 BE071985 BE071945 AW577383 AW749830 BE072005 BE071965 BG955674 AW577355 BI837879 BG389090 AA071273 |
| | | BE266655 |
| 414754 | 1165452_1 | AW938668 AA152116 BE000477 |
| 443073 | 590566_1 | AI280093 AI032321 AW082160 |
| 415641 | 1877037_1 | H14742 Z44481 F13256 T77036 |
| 440534 | 1049846_2 | BE561168 BE269996 AL561649 |
| 420968 | 264225_1 | AW968775 AW662892 AW592439 AW663117 BF353890 AA804580 AA834616 AA282186 |
| 443197 | 246171_1 | BF104862 AW954963 Z43613 AA320191 R83872 H15889 |
| 418330 | 36528_1 | AK056357 BG704214 BG714527 BE409405 BE514455 BE408705 BM351826 BM049253 BM352137 AI659121 AW074357 AI674368 |
| | | AW207841 BE646201 BM453984 BE674342 H19667 AI458175 AA279585 AW438632 AI687141 AA433999 AI245284 AA366782 AI948491 |
| | | AW450917 AW450396 AI949813 BE217772 BE504863 AI628938 BF059575 AI564121 AI073854 AW510570 AI769861 AW235541 |
| | | AW771224 AW236106 AW074675 AI919535 AA977064 AI364994 AI631614 W58249 AI034276 BE169956 AA743584 H19668 AI222131 |
| | | AA883261 AI351534 F10472 AW466882 AA814964 W58250 BF036304 BF932025 BM474362 BG110744 AW964083 AW369992 F12868 BF842785 AA356665 BI091945 |
| | | BG059147 W04844 R18398 BE925184 BI050068 BI825078 BE784883 AW175845 AA216130 BI257636 |
| | | AA504328 AA713664 BF754840 AW235144 R38555 R42461 BG676504 T75135 R15365 BM012684 |
| 426105 | 1227975_1 | AW962732 AA369895 AA370773 AA369894 |
| 431698 | 6468_7 | BM423504 BE075913 AI492369 AI831729 AA807134 AI057292 BF059751 AA918209 BE858647 AW016707 AI675289 AI452916 BF593014 |
| | | AI480099 AI522283 AI628681 AI470402 BF056360 AW236830 AA514206 BF055812 AI796201 BF055617 BF054827 AI288301 AA705763 |
| 431317 | 997174_1 | AW970601 AW613399 AA503435 AA502682 N91138 |
| 459057 | 40191_3 | AI808408 AW005512 |
| 415204 | 1865508_1 | Z25288 R85077 F00323 D82802 T27434 |
| 449158 | 3025931_1 | AI990849 AI652180 AI632577 |
| 459557 | 859794_1 | BG623239 N58315 AI524952 |
| 454645 | 1065291_1 | AW811928 AW811887 AW811948 AW811847 |
| 411226 | 1073516_1 | T62567 AW833022 AW833054 |
| 450724 | 40161_1 | BF060834 BI019048 AW862002 AK056737 AA429538 AW063311 AW440655 AW231970 AA428584 AA995028 AW854593 AA984131 |
| | | AA552874 AA564758 AA528743 BG927275 BE045117 AW975234 AA664937 BF749390 BG928948 AA984546 BE009153 AV732487 |
| | | BE078167 AW872716 AW063245 AA578441 AI820704 AI732283 R55428 BF999154 BF880485 |
| 414205 | 1266364_1 | BF306238 BE295439 BE382708 BE383262 BF315437 BI199567 BE382438 |
| 414883 | 8371_2 | AF274943 BG494894 AI719075 AA908783 AI935150 AI422691 AA910644 AA583187 BM272167 AI828996 AA527373 AW972459 AI831360 |
| | | AA772418 AI033892 AA100926 AU154749 AI459432 AI423513 AI094597 AA740817 AI991988 AI090262 AI312104 BI256707 AA459522 |
| | | AA416871 AI075239 AI339996 AA701623 AI139549 AI336880 AA633648 AI989380 AI362835 AA399239 AI146955 BF514270 N92892 |
| | | AI348243 AI278887 AA459292 AI494230 BF507531 AI492600 AA962596 AW613002 AA293140 AA235549 BF108854 AA954344 N49682 |
| | | AI457100 AW589407 AW300758 BE220715 BE220698 BE569091 BM009647 BF900351 AI537692 AI203723 AI857576 AA584410 |
| | | AW371667 BM172363 |
| 425265 | 921293_1 | AW505023 BE245297 AA353976 |
| 412124 | 32402_3 | BI089431 AW027705 BF377448 BF377461 BF377453 AW967810 AI341165 AI652171 AI262672 AI021922 AA564575 BG939545 AI041471 |
| | | AI350656 AA243478 AL079653 BF885008 BF092841 BF377464 BF377463 BF377444 BF092882 BF792973 BF377460 BI089398 AA627599 |
| | | N76045 |
| 452482 | 1524509_1 | BF350690 AI903640 BF350532 BF350688 BE153688 BE153461 BF350692 BE155413 BE155356 BE153488 BE155063 BF350538 |
| | | BE155210 BE155059 BE155347 BE155492 BE153577 |
| 452146 | 627690_1 | BE795675 AW206850 AW135654 BF063909 AI862440 AW401701 |
| 454204 | 646158_1 | AW816498 BF374419 BF374408 BF374405 AW808977 AW808605 BF334681 BF348941 BF348944 AW178676 BF374412 AW178486 |
| | | BF374427 BF374429 BF348942 BF374428 BF348955 BF348940 BF348943 BF374416 BF374424 BF374431 BF374430 AW808524 |
| | | BF374413 BF374418 BF334708 BF374389 BF334685 BF374473 BF374392 BF374397 BF374395 BF374407 BF374417 BF374420 |
| | | BF374414 BF374422 BF374421 BF374522 BF349306 AW808532 BF374399 BF374393 BF374398 BF374394 BF374396 AW178485 BF374391 AW808816 BF374516 |
| | | AW178483 AW808515 AW808791 BF374390 BF374415 AW808514 AW808379 BF374423 BF374426 BF348949 AW809007 |
| 409078 | 744783_1 | AW327515 AW327774 AW327571 |
| 411688 | 1097434_1 | AW953440 AW857085 T08189 |
| 437863 | 2513792_1 | AA923707 AA769989 AI393571 AA972997 AI951436 |
| 409872 | 917143_1 | AW502313 AW502681 AW502682 |
| 440612 | 1026055_1 | BE561384 AW732707 BE396946 BE513448 |
| 455540 | 430605_1 | AW993000 AV720038 AV720142 AW993293 BE080231 AW993284 |
| 438171 | 143478_1 | BF966744 AW236835 BG204580 BG220785 AW631476 BG221281 BG183393 AI816071 AW157314 BG189742 BG201352 BG188096 |
| | | BG216769 BG216076 AW261874 AW151173 AW001897 AI801254 N62855 AA779526 BG461073 BG181264 BG213529 AW976507 |
| | | AW235694 AA876913 AW117364 AW150293 AI635914 AI584106 AW148443 D12158 AI245076 R40658 D57788 BG189741 AW166670 |
| | | D12218 |
| 421828 | 207_22 | AW891965 AW604749 BE080872 R15559 BE177623 AW883520 AW945343 AI246167 T07082 AW805679 W96278 AA135796 W32615 |
| | | AW995418 AW801688 BE003837 AW801621 AW385721 AW385742 AW385714 AW604757 W87409 AW604738 AW385757 AW580796 |
| | | AW801247 BE003239 BE003183 AA847112 AW580975 AW604760 AW385727 BE164590 BE003090 AW362791 AW604759 AW866589 |
| | | AW604758 N44337 AI378548 AW890438 AA077172 AI288683 AA229639 AA091945 AW945454 AA063629 AA702504 AW861938 |
| | | AW894816 AW580841 AA094372 T06399 AW885686 BE244086 BE005035 AW861913 AA551773 AW858460 AW370926 AW754352 |
| | | AW889695 AW384408 AI907428 BE067491 AW861939 AA248197 AW381373 AW177325 AW806879 AA935217 BE067498 BE083742 |
| | | BE067470 AW894935 BE082529 AI248811 BE179917 BE002200 AW607506 AW392889 AW894560 AW381360 AI904206 AW863533 C00609 AW381372 BE082530 AW898120 |
| | | BE075323 AW392799 AW601420 AI695314 BE083790 AW858568 AW945550 BE177153 |
| | | AW970506 BE350419 AI906919 AW360794 AI906917 AW885979 AW794240 AW945566 AI688683 AI688694 AW009660 AW601421 |
| | | AW360793 BE066524 BE083901 AW369847 AW381871 AW935435 AW664582 AW877775 AW838449 BE180466 AW858501 BE180464 |
| | | AI371163 AA778231 AI174991 BE011720 AW877776 AW877800 AW877795 T19900 AW866365 AW898099 BE011715 BE167842 |
| | | BE011718 BE011724 AW363639 AW878658 AW878662 AW894887 BE082356 AW389211 AW804286 AW610312 AI904717 AW610318 |
| | | AW996909 AW610296 AW901923 AW880003 AI762171 AW062582 AW368713 AW062593 AW176663 AW842064 AW842089 AW842095 |
| | | AI243049 AW902074 AW062592 AW176664 AW751692 BE087703 AI907439 BE009686 BE172115 BE077030 AW608556 AW835577 |
| | | AI909628 BE077029 AW176241 BE077552 BE160370 BE160288 AW835656 AW606765 AW606770 AW835678 AW606758 AW606778 |
| | | AI907484 BE172821 AW606768 AW999517 AW844165 BE171738 AW751683 AW610493 BE177484 BE177487 AA090510 AW844117 |
| | | BE173367 AW999878 AI124870 BE163472 AW841823 AW379762 AW893297 AI290296 BE089132 AA610287 AW176676 AW607622 |
| | | BE172639 AW893232 AA329629 BE089008 BE178350 BE178214 BE063291 AW820236 AW999653 BE089486 BE173126 BE171775 |
| | | BE185787 AA558280 AI174840 AW999112 BE218391 BE172734 BE178021 BE172738 BE173324 AW603494 AL036722 R38192 R60905 H53721 H41052 AL037917 |
| | | R37795 AW998972 AA767189 AW044272 H50689 AA768399 AA767764 AI087888 H44202 BE222792 N90597 |
| | | W81396 N90615 AI935353 BE501168 F10945 AW118215 AI970480 AI627641 AW236081 AA574090 AI627652 AI681913 AI759983 N69591 |
| | | N69276 BE467722 AW392780 BE172467 H92861 AI524921 F02989 Z39328 F02705 F01414 T88678 AI215165 H87220 AW374781 |
| 454560 | 1061353_1 | AW807092 AW807171 AW807174 AW807425 AW807274 AW807281 AW807278 AW807367 AW807330 |
| 411942 | 1142439_1 | AW877069 AW877063 AW877071 BF361321 AW876988 AW877015 AW877133 AW877013 BF361309 AW876978 |
| 425702 | 1283939_1 | N59555 AA362113 AA362136 |
| 450754 | 665849_1 | AW204237 AI793326 AI733299 |
| 411782 | 1105929_−1 | H30518 |
| 442481 | 678752_1 | BG398943 N99828 AI110738 AF074645 BE969969 BE079873 BF185244 |
| 422285 | 428504_1 | AL525104 AL525103 AI347488 AI659881 BF509572 AI803103 AW470793 AW450703 AI885143 BF445866 BF221925 BF002607 AI090784 |
| | | AW242783 AW271587 |
| 444326 | 2795105_1 | AI939357 AI992056 AI140955 AI198235 |
| 442430 | 59995_1 | AK057699 AV654749 H77767 BE674322 AA995737 R89164 BF831094 |
| 417046 | 2446559_1 | AA192639 Z28835 |
| 400234 | 3084_1 | BC001179 NM_005336 M64098 BC014305 BI827845 BI918183 AU124191 AU125604 AU140930 AL043307 BG763670 BG824279 |
| | | AU125959 BI518466 BE271867 BM264138 BE314823 BM090633 BE273657 BG419830 BM048338 AU134368 BE163486 BE163483 |
| | | BG421980 BG768130 AW500482 AI910107 AW868323 BF083170 AW450290 BM466817 BG255224 BE394282 AU140278 BE887464 |
| | | AL042675 BI195986 BE883385 BE544751 BE270148 BM011334 AI093482 AA029803 AI610705 BE386809 F06628 AW408605 BF814085 |
| | | BC007961 BM451548 BG768053 BM452948 AU140330 BG489566 BI870474 BM014013 AU131264 BG679824 BI225752 BI457400 |
| | | BE410173 BM044254 BG763690 BE251495 BG179862 BE513489 BM005937 AI064815 BF569858 BI712614 BG655891 BG424940 |
| | | BE175180 BM313160 BG471936 BM091124 BF885884 BF870522 BE004490 BE712976 BE712862 AA401033 BF569477 BE712936 |
| | | BF353839 BE713042 AA316561 BE712974 BE712977 BE712938 BE712937 BE712867 BE713043 BE713023 N42463 BE712901 BE713041 |
| | | BE712904 BE712895 BE713012 BE713049 BE713025 BE712998 BE712988 BE712987 BE712986 BE712984 BE712982 BE712975 BE712970 BE712953 BE712929 |
| | | BE712922 BE712875 BE712866 BE712861 BE712860 BE712854 BE712853 BE712902 BE712897 |
| | | BE712912 BG984910 BE712903 BE764759 BE715723 BE715745 BE712915 AA441839 BE713040 BE715755 BF872528 BF060840 |
| | | BG395104 BG744878 AU124081 AI889823 AI801072 AI633102 AI678670 AI493608 AW194294 AI354559 AI686736 AW152477 AI419496 |
| | | AI720319 AI866190 AA047013 AA039625 BE049622 AA369065 AA025686 AI570198 AA706304 AW439251 H64553 AW168006 AI983753 |
| | | AW081999 AW082909 AI951490 BF886341 AW591246 AI190143 AI151074 AI499541 AI050934 AA402167 AA382049 T16207 T03299 |
| | | AI932691 AW571663 BG479797 BE408436 BI460153 BG386046 BE616601 AW151629 BG236164 BF852743 BF852046 T57801 W37981 |
| | | AA482668 H43740 W58142 W25087 BM459852 AU125403 BG767992 BE258516 AI114509 BE394494 BE386905 AA450315 BG750593 |
| | | AA557855 BG766487 BG766998 BF948864 BE279327 BE870900 AA577098 BF365851 AA745058 N29867 AI625038 AA641801 AA846174 |
| | | H26395 AA025568 BF690282 R67845 R72666 H39554 T78959 W01317 H45860 W80837 N53985 BG757202 BE838055 BE072895 |
| | | BE072668 BE867012 BM042833 AA083591 BG745462 AW374086 BG831326 BE715757 BG767243 BG763272 AA113402 AI024044 |
| | | BF818501 BE081938 BF353818 AA746336 BM019086 BE712894 H16977 H45012 R10793 BE708619 BF570432 AA593938 AA064637 |
| | | T28082 AA852845 T78907 AA346590 F00017 BE966368 N58726 AA732431 T54379 W80749 BF238314 BE279768 AA325106 T19241 |
| | | BE409939 BF852119 W23803 T54011 AW392638 BI465185 AW516784 AA211335 BI159846 AA064680 N80615 W49676 BF851406 |
| | | R88120 AA434028 H44546 T61029 N44804 AA114948 H26960 BI261965 AA582599 BG469385 BF818172 BF923672 BI024270 AW362633 |
| | | BE883122 BM470779 BE935760 AW856056 AW856103 BE828607 AW381799 AL121058 BE828618 BE769545 AA102142 BG749978 |
| | | BI770417 BI862544 AI110687 AF063562 BE740347 BE874864 AI305113 BE513675 BE091369 BI046660 BE091453 BI046548 AI673786 BF868704 AI025179 BF356840 |
| | | BF886357 BF886313 BG951884 AI347880 AW627563 BF932536 AW820377 BF739372 BF831620 |
| | | BE068264 BE613627 AA641745 W01130 H28252 |
| 434206 | 185910_1 | BF591031 AW205438 AA933791 AI434866 BF513564 AI191480 AI972330 AI582824 AW779392 AI359970 AI199117 AI910783 BF800971 |
| | | AA627401 AI799778 BF222639 AI937737 AW136973 |
| 409960 | 39576_1 | BE644758 AI082238 BF940027 AI201079 AI436035 AW275966 AI085394 AI291655 AW070441 AI474134 AI268978 AI769279 AI567682 |
| | | AA693941 BF477668 AW664149 AA283782 BF509538 AW296868 AI268977 AI168133 BM352065 AI262769 BF941976 AI056920 |
| | | AA481861 BF763697 AL565888 BM352383 AA427768 AA385346 AI186988 AA931831 AA134972 BF217480 BF111012 AA908246 |
| | | AA319849 AA318136 AL514271 BF364291 AL515057 AV702312 AA377395 AL544217 AI341000 AW193583 AI350789 AA888338 |
| | | BF945380 AW879092 AA130839 T91066 N92326 AI004389 AA078832 AL572370 W04622 BE314003 AW960808 BM360872 AA319160 |
| | | AA130778 AL514257 |
| 451617 | 86010_1 | AI346282 AI095742 AW264306 AW264209 AI582421 AI536545 AW503193 AW134845 AA563713 AW074239 AA018842 AI807383 |
| | | BG060054 AA716066 BI042559 W37890 AI375422 N23383 C01056 AA772036 BF953159 |
| 409189 | 108922_1 | AA075168 AA070377 BG876547 N84829 AA065075 AA070346 AA085208 AA082764 AA083380 AA076512 AA113255 AA100017 |
| | | AA125984 AA079891 |
| 455618 | 354455_1 | BE155574 BE155563 BE155556 BF988228 BE061294 |
| 437063 | 10748_1 | AK000711 AV706281 AI990891 BM021502 AI198420 AW135128 AW296896 AI972531 BF433758 AA972785 AA912863 AI699929 |
| | | BM353878 AA446409 AL047908 AW664908 AI768280 AI418839 AI379964 AI399807 AA435632 AI383321 AA256372 AA459268 AW512463 |
| | | AW015449 AI961893 AA748516 AA448386 AI560461 AA465229 N41016 T24027 AA447151 N46418 AI393430 AA565928 AW468864 |
| | | AA632571 AA904477 AA436438 AA446105 AA012996 D62192 AI382506 R40297 AV704817 AA459497 AA393443 AA663571 AA448383 |
| | | AA256888 BM021226 AI909083 AA429833 AA299114 AA034292 AW291004 H85814 AA012995 H56165 AL047907 AI339983 AW074407 |
| | | N93781 AA351108 D79494 BF510070 AI419381 N56890 N67978 AV706037 BF572871 BE972375 H56381 |
| 400203 | 11774_1 | NM_002794 D26599 BM469989 BF305151 BG821966 BI089030 AW007738 BI222910 BM049422 BG028749 AI189162 AI831230 |
| | | AW131497 BM272215 BE791105 AW778828 AA479594 AA480133 AA131997 AA284572 AA453009 BF928258 AA152127 AA393918 |
| | | BF765307 BE247542 BF934697 BF341798 BE253409 AA470620 AI828932 AW379902 AV762678 AV741784 AV760892 AI025755 |
| | | AA878562 AA630630 AA761708 AA862518 AA865831 AA862947 N53065 AA131821 AA293499 N23342 N26856 AI147346 AW951549 |
| | | AA772963 BE245986 BG208493 AI831666 BG474873 BI023168 AU149647 BG197069 BG191102 BF304178 BE536135 AA706900 |
| | | AA443583 AI002710 AW276192 AU149842 BG214797 BG198193 AW197923 AW627799 T98663 BG194788 BG214656 BF345258 |
| | | BG716363 AI066528 BI546220 AA393815 AA132004 AA353826 H97858 BG187823 BF841463 AI351714 AV735966 BG196439 BG216840 |
| | | BG198438 BG400762 |
| 434878 | 119973_1 | AW975086 AA652771 AA658179 AI494481 |
| 447197 | 2176805_1 | R36075 R36167 AI366546 |
| 459376 | 31010_1 | BC002465 BE254864 BG472164 BE258770 |
| 455475 | 1175177_1 | AW948153 AW948139 AW948124 AW948125 AW948131 AW948145 AW948164 AW948134 AW948127 AW948162 AW948157 AW948151 |
| | | AW948196 AW948126 AW948158 |
| 440129 | 2607882_1 | AI732997 AA977633 AA865818 |
| 410859 | 1061852_1 | AW807671 AW807841 AW807914 AW807676 AW807920 AW807604 AW807905 AW807765 |
| 443108 | 2431141_1 | AI829905 AI033329 W86975 |
| 411436 | 1083231_1 | AW846159 AW846377 AW846433 AW846528 |
| 448501 | 8235_1 | BF963346 BI460763 BI599382 AI188089 AY055003 AW959185 AA187681 AI692282 AU153608 BI493898 BI493899 AI797349 BE550679 |
| | | AI701137 AA744561 AA082682 BE218816 AW511501 BF447881 AI859101 BF059295 AI914038 AI346564 AI685003 AI676076 AI125336 |
| | | AW469637 T10225 AI831296 R53451 AI453440 AA983739 AW470873 AI348290 BE857670 D55901 Z43908 T34429 F07305 AK074340 |
| | | AL538118 BG201484 AI334192 T35535 AW176751 BI496132 BI496133 AA469961 H72176 BF326265 AI770016 AI693177 BF223634 |
| | | BF963661 BF962265 BI034894 AI475851 N69071 AI910707 BE219038 AI918036 BE670589 BM193515 AI338497 AA805525 AA487625 |
| | | AA830336 AA749368 AW104323 AI628055 AI932332 AI272788 AI536849 AW162414 AW161923 T23854 AA610763 AA912188 AW339028 Z39946 T10224 F03171 AI205478 |
| | | AI638791 F02580 F01551 AW207551 AA421030 BE246012 BI034937 BI035373 BF939581 H19984 |
| | | BE867247 H92677 N55988 BF957332 H18615 R16442 BF956229 BF961886 R12698 AA101186 AA365932 R53452 BF062714 BF959364 |
| | | H41634 AW086187 H19985 BF054881 F02581 H18616 H41527 AW003446 BE243443 H92276 AW954002 BI670132 |
| 422890 | 61426_1 | AK057805 AW162343 AI190479 AI093318 BE048820 AI198397 AA654667 BE219303 Z39851 F02655 Z28734 T16575 F10145 Z45266 |
| | | AW572911 AW964436 AW004030 AI632565 BE502530 BI792383 BF056928 AA449241 AI651825 AA805324 AI264863 AW196918 |
| | | AA948267 AI953735 AI263703 BF056387 AW594171 AI867447 AA319159 AI903440 AW956110 AI366013 AI867923 BG911906 D81142 |
| | | C15616 AL538697 Z25032 Z43784 R13382 AV746924 AA449369 AA318815 BF364265 H17038 H10064 F04161 T87230 R40898 |
| | | AW204071 BI819428 AA683393 AA683376 Z43192 T74078 T05103 F12527 T77951 H10118 H17037 BF855407 R19603 |
| 438814 | 641599_1 | AA883216 AW170014 AA826278 |
| 437838 | 2512601_1 | AI308202 AI307229 AA769348 |
| 411911 | 1141625_1 | AW875951 AW875950 AW875939 AW875957 AW875948 AW875936 |
| 455299 | 364754_1 | AW891790 AW891797 BG003264 BG001459 BG002583 AW891612 |
| 410600 | 497855_1 | BF347859 AW499616 AA191322 AW499617 AL601010 AW575742 AA729043 BE463447 AA086179 BE549623 AI335824 AW408712 |
| | | BM149172 |
| 444298 | 67578_4 | AL039867 AI139927 Z17870 |
| 440035 | 359_4 | BC018584 NM_021831 AK025492 BI711294 AW574711 AI819256 BC018562 BM009183 AW973895 AI379616 AI333037 BI259314 |
| | | AI039603 AI085721 AI334184 AI351665 AI355677 AI399638 AI378200 AA150445 AI955108 AA827596 BF508487 T91077 AI919430 |
| | | BF434608 AA193689 AL532354 AW080055 AA860369 AI143451 BG744632 BF822458 AA707356 AA035097 N20028 AI620258 W79520 |
| | | AI471067 AL577305 BG750060 BE513499 BE268712 BE560765 BE540972 BI829285 BG720872 AA190575 BM014928 AA035096 |
| | | BE870633 BI226012 BI222402 BG718729 BM044958 BG718967 BI085526 BI561896 W27694 AA156945 AI041365 AI520777 AI919222 |
| | | AA781071 AA152279 AA594545 AA861397 AL522279 AA632356 W79407 BG164415 |
| 400120 | 133_1 | BC014030 NM_004068 BC004996 AK057883 D63475 BM468205 BG386792 BG750447 BG575842 BG479084 BG741027 AU118129 |
| | | BE901043 AU141281 BG825395 BG338276 BE396231 AU121493 AU131489 BF341132 BG335659 BI769251 AU142779 AU124483 |
| | | BG480828 AU121353 BG702326 AU137866 BG759046 BI460601 BE887290 BG750415 BF127710 BG757819 BI160266 AU122086 |
| | | BG824046 BG744180 BE884943 BM006610 BG702599 BI870749 BF307033 BI334771 BG480606 AU142599 AU141336 BM018563 |
| | | BI335565 AU138308 BE391241 BG774488 BE278025 BG717959 BG706920 BE312567 AU138300 BE303034 BE303016 AU139252 |
| | | BE336797 BG470940 BE336735 BF305197 AU134288 AU139907 BE261245 BE208718 BG761912 BF212890 AW732692 BM011258 |
| | | AW247629 AU136696 BF530078 BF204146 AL048752 BG328927 BE388385 BE260122 AU138789 BE253465 BF733914 AW249415 |
| | | AW239535 BE311791 BE256236 BF529742 BG770465 AW245777 AW245813 BE296677 BE266852 BE168115 BE396596 BE280057 |
| | | BE168229 AI750820 AU134137 BF792191 AI272215 AI907348 AW238875 BF805152 BF568397 BE712727 BE081443 BE001805 |
| | | BF724536 BF744705 AW247385 BE796369 AU133759 AV705142 BE794402 BM465821 BG281284 AW384831 BM450689 AU134125 |
| | | BE311650 BF356318 BM462831 BG420555 BE749127 AU134590 BM019438 BC013796 BG761223 BG122058 BE872076 BG748496 BG821374 BE619159 BG423244 AU137110 |
| | | AU127210 BE270081 AA496860 AA351380 AA356303 AU099781 AA355912 AA371411 |
| | | AA325535 U36188 AL569574 AL577204 AL525543 AL567342 AL567334 AL567122 AL536527 AL567098 BG681585 BG824951 BI870652 |
| | | BI225855 AW393878 BG750632 BI223803 BE877552 BE270473 BE389392 BE779021 BM017845 BI117816 BE396513 BM049006 |
| | | AW393945 BE786941 BE267724 BE562981 BE314236 BF744102 BM019781 BF806063 BF828673 BF829181 BI008788 BF829180 |
| | | BF829175 BF433802 BE909928 BF839784 AW578564 BF943095 AW328030 AA448596 AW882688 AA909846 BF924341 AW361460 |
| | | AA425174 BE940557 BF761585 BF931276 BF934886 W68597 BF933977 BI035906 BE836064 BF837576 BI018368 AA205908 H54612 |
| | | R88902 BE812330 BE932300 BF924562 AW805376 BE769859 R87381 AA371901 BE838855 AA326381 W56191 AA341231 AA464093 |
| | | AL575977 AL518650 AL547393 BG338327 AL517563 AL536921 BG207096 BG207589 BG199290 BG220159 BG744842 BG104730 |
| | | AA555035 AA618009 AA861062 AA610582 AW245418 AI040983 AA521380 AW245455 AU147292 AU155236 BE620286 AW250767 |
| | | BF888236 BE620819 AU154343 AU150827 AU148334 AW069495 AA701091 AU155225 AW250019 AA666235 AU147764 AA449739 |
| | | BF907598 AW043731 AU144390 AI924565 BE300631 AW874021 BE208088 BF732773 BF594057 AI159873 AU143930 N24100 AI052499 |
| | | AI989370 AI366151 AI139248 AU144776 AA838250 AI095433 AI269227 BF811358 AU151352 AA477554 AI131290 AI049797 AU157349 |
| | | AA620559 AA581533 AI338299 AA402755 AI050079 AU157614 AI280691 AA706590 BE206615 AA861056 AU153214 AU155712 |
| | | AW069554 AI081124 AU155846 AI081123 AA719876 AI754976 AI027763 AA723095 BI518782 AW050706 F37521 R79062 AA565530 H39826 AA404670 AU158884 |
| | | AI139682 AA513633 AI283202 AA651856 AI623515 AA427413 AW248474 BF841432 BM453246 AI302390 |
| | | AA808269 AI249357 AA132775 AA716732 AA479932 AI198296 AI208674 AI088433 F36977 AA496899 AW615762 AA602541 AA341161 |
| | | D58785 T15399 AI286077 W69152 F28418 AI272156 AA338034 AA568455 BE206121 AI080033 AI699680 AA861255 AI953465 AI613240 |
| | | AL581773 AL531577 BF526361 AA657809 AW152670 BE621523 AW078705 AW673719 AA102613 AA195604 R72209 AA402208 |
| | | AA404305 N24999 AI963535 D54741 BI461415 BM475959 BI260926 BI116213 BE280120 BE940258 BE280044 AI905744 BG001474 |
| | | BE937718 BF799283 BI091621 BG421006 BG289235 BF736825 BI253429 BG170064 R72512 T92815 BF931257 BM455183 BG033362 |
| | | BG574220 N31395 BF739185 AA371995 BE879011 BI198754 BF378989 T12266 BM019421 BI194570 BF378992 AW996595 BI858649 |
| | | W94605 W61345 AA122384 AA171923 T92736 AA761504 AI819039 AI439358 AL517777 AL565919 AA622001 AA642695 AA704144 |
| | | AA081465 AA070621 AA375562 AA700011 AA926863 W37310 AL566236 BF677809 BG760021 AW361433 BE828605 BE268449 |
| | | BF805977 BG292452 BF981071 BF217108 BF928698 R33993 AW882841 AI857453 AW078733 AI433035 AI018103 R90927 AA804720 |
| | | AA551734 R33835 H19741 R78754 BF930494 BG109583 AA631926 BE834008 BG996533 |
| 429462 | 5761_2 | BC008352 BM006782 AW245715 AI890356 BF445407 BE222242 AW139455 BE549685 AA987521 AA960934 AW245359 AA453285 |
| | | AA976507 BG055359 BM007094 |
| 446830 | 41421_1 | BC020595 BI488430 BG168023 BE179030 AW294203 BF849776 AA459064 AI917452 AW403072 W27419 BF914568 BF798468 |
| | | AW370558 T35055 AW370623 AA399232 AA214221 AW802987 BF902228 AW370622 BF819597 AW370567 BF914313 AW954040 |
| | | BF060706 AA194237 T25074 C01285 BI489433 |
| 400125 | 9675_1 | D78361 D87914 BC013037 BC010063 BC007247 BM463837 BG478630 BM463154 BI666615 BM459684 BM453662 BM453786 BM047472 |
| | | BE900180 BF343030 BI600045 BG755862 BM019670 BI596806 BI334555 BI596686 BG703983 BG338513 BM016325 BG701724 BI602310 |
| | | BF974248 BG825153 BI601788 BG826747 BG823109 BM043228 BE792227 BE276371 BE619490 BG699290 BE875106 BG764897 |
| | | BE297371 BI083984 BI261435 AV708008 BM020241 BG769564 BE276363 BE782631 BE621029 BE269266 BM009681 BG752384 |
| | | BG708932 BE264756 BG822294 AV655728 BM010141 BG764961 BG708520 BE882611 BI225883 BG705799 AV685946 BG257658 |
| | | BI598660 BE304415 AV702447 BG748238 BE269385 BE383094 BE266448 BI868791 BG826478 BG423317 BG033769 BE265113 |
| | | BE270094 BG423167 BI334713 BF311958 BE385530 BE262622 BG576563 BE882639 BG939261 BE296716 AV702822 BE292912 |
| | | BE385748 AW404766 AA313782 BE884711 BE781140 BE397059 AL121483 AW405810 BE264213 BI438381 BE264869 BG686776 |
| | | BI667400 AW950282 AA307682 AW248234 BE513268 BE279880 BE269204 BE396504 BG419840 BF725176 BE900202 AA308557 |
| | | BF725611 BI549558 AW575370 BM272866 |
| | | BE551344 BF725743 AW406696 AI401132 AW575527 BM312069 AW406436 BF725705 |
| | | BM272877 BE302894 AA309714 AI871787 BF347516 BE408306 BE314993 BE267390 BI908563 BI837140 BI821837 BI521207 BI837505 |
| | | BI822161 AA317001 BE207225 BI754627 AI689750 BI909534 BI764695 BE263483 BI906501 BF149297 AW246261 AW406823 AW406016 |
| | | AI792406 AW405766 AW247989 BG655937 AW406365 AI929708 AW406994 AW404540 AW732985 AA351302 AI299658 AA311847 |
| | | AW406963 AA312048 AW404396 AA374650 BF025776 AA302326 AW404904 AA233498 AA371786 AA360608 AA352537 AI904689 |
| | | AA296974 AW405056 AA381543 AA381946 AW369724 AA345437 BE163548 BE163476 AW996790 AW901359 BE709949 BE709922 |
| | | BE797158 AL517239 AL542136 AV752296 AA302909 AI469136 AA569581 AA321755 AI753370 AW769988 BG152412 N79811 AI524667 |
| | | AI028654 N75351 AI971354 AA873772 BM142020 N93051 BM141746 AI819519 BF448393 AW131088 AI680121 BF940970 BI468461 |
| | | N64809 H81687 AI932580 W72498 AI418617 H97476 AI634888 W81227 N92886 N69882 R51087 R28305 AI623535 F01410 BF216926 |
| | | BI060242 AI991320 AA573813 AA357272 AA778588 F22071 AI500046 T34044 F19132 AI914838 AI688763 AW969985 BF882549 |
| | | AI191071 AW473453 AI500047 AI860191 AI439085 BG939534 AI469726 R89388 D20006 AA729773 AL542476 BE162911 AA010461 |
| | | BI160800 AV704564 AI660824 AI345965 AI588895 F17300 AA579720 AA570197 AA865991 AW149676 BE272153 AW055113 T29965 BI060387 BF916086 AW090265 |
| | | AA351301 AI922370 N24035 AW149561 AW117762 T59861 BG481492 AA659904 W93933 H89803 |
| | | AA858115 W47305 BE925168 BE395221 BE537366 AI151066 |
| 446457 | 2871742_1 | AI949435 AI921903 AI300580 |
| 454923 | 1082210_1 | AW845406 AW897236 |
| 418647 | 243680_1 | AA226513 AA383773 AA226198 |
| 455642 | 1489271_1 | BE063966 BE064034 BE064028 BE063869 BE064033 BE063968 BE063965 BE063874 BE064043 BE063884 |
| 457146 | 881_1 | BG426090 AW071802 BF058083 AV723821 BG433915 AI672895 AI818871 AI017491 AA421820 AA284792 AI418120 AA558952 AA910750 |
| | | AI268326 BG613224 BG325068 AV724733 R54850 AA973795 AA171449 BF883665 BF883667 N46097 |
| 436314 | 142774_1 | AA708982 AI095911 AI983409 |
| 407347 | 810943_1 | T23514 AI655785 |
| 454515 | 1058442_1 | BE062940 BE079700 AW803201 |
| 458744 | 827170_1 | AW445183 AI380746 |
| 419557 | 149562_1 | N94706 BG536024 BF824886 W01777 AI949662 N50068 AA917338 BE550711 BF739895 AI340079 AA971513 AI467976 AI694919 |
| | | AW206626 BE328279 AI310369 AI307346 AI862813 AA425773 AW475016 AI932791 AI567886 BM128600 BM128547 AA621126 |
| | | AA244453 AA369087 BM128368 BM128327 AA628674 BF223957 AI678489 H10786 AI857652 AA502988 N63025 AA244420 |
| 415989 | 10194_1 | BC013389 BC017398 AI023543 AA191424 AI267700 AI469633 AW958465 AW953397 AA172056 BE940298 BF909208 BF909980 |
| | | BF095153 BG285837 AI720344 BF541715 AA355086 AA172236 |
| 447460 | 22756_3 | BC015313 BG104940 AI380282 AW872495 AI436655 BF439817 BF109066 |
| 448889 | 1514989_1 | AI587189 AI816573 BE140902 |
| 439117 | 4929_1 | AF085975 H53459 H53458 |
| 430539 | 31268_1 | AK001489 AU129447 BF959274 BG565452 AI245327 AU116848 BF358559 BF358554 BF358570 BG678119 AL515852 AU154607 |
| | | AI357567 AW874359 AI122554 AA406478 AI091013 AI866679 AI686163 AA662158 AA911580 D31095 AI302576 BF588761 AU151560 |
| | | AU143828 AI291610 AW169600 D31161 AA905362 C21179 BE327258 D31474 AW439053 D31309 BF756901 BI838626 BF979839 |
| | | AU149562 BM142116 AU156455 AA452028 AW473972 AW468490 AA410271 AI475944 BF821859 AA658188 AI360390 AA226320 F37355 |
| | | F27660 F36093 AA152126 BF930021 BF375775 AW821784 AW975085 W16475 D31031 BG696392 AW860676 AW752864 BI013705 |
| | | BF965715 BF326604 AW821786 |
| 443784 | 82683_1 | BF970444 AL040806 BF829540 AW612344 BF829549 W78883 AV752303 AA355882 AA286965 H83215 BE895370 AI191264 N98817 |
| | | R16590 BE793490 BE621572 H25334 H26704 AA074589 AA194985 AI084640 AI040923 AI367435 AI187367 AI127094 AA132771 |
| | | AI273475 AA553397 AA620288 AA287968 H82982 AW298586 AA765121 AA405226 AA406125 AA722863 AA860925 BF515351 F11046 |
| | | AA992696 R89635 AA075266 AW058095 W84657 H26626 AA575897 AA836488 W88453 X96619 W90020 H25291 R16533 T54676 |
| | | H26705 H89489 H89635 BE828780 BF888069 AA216309 BF091562 H26680 BE259933 |
| 429438 | 30460_1 | NA |
| 412700 | 18692_7 | BG164322 BF510268 AI681542 BE222433 AI299965 BE220552 AA115728 BE896438 |
| 408756 | MH1740_17 | AA524743 BF515403 BM353526 AI803357 BF000688 AW665479 AI672715 AW150325 AW589318 AI693038 AI370572 AI167636 |
| | | AA057236 AW439079 AI097376 AI382060 AI684871 AI684860 AA913419 AA806162 AI673426 BI324967 BM149631 BM145789 BM353796 |
| | | AA913878 BM148707 N38884 BI761934 BM145978 BE243268 BE246569 N38883 |
| 417952 | 75065_1 | AK024870 AW361863 AA132516 AA047268 AA931767 AA349517 AW090602 H18429 AW979065 AW360829 H07105 AI192838 AI866559 |
| | | AA405498 BM148597 BG391026 AA210693 BG503033 AI764977 AI167261 AA210812 AA834832 AA829145 AI824218 AA595531 |
| | | BG539322 AW962918 BG957109 BG956751 AA369687 BF805524 AW798909 AA303140 AI216690 BG957010 H06593 AA303141 |
| | | BM153757 BF238864 |
| 454631 | 1064852_2 | AW811332 AW811335 AW811328 AW811329 AW811339 |
| 454171 | 1049240_1 | AW854832 AW854798 AW854857 AW854816 AW854834 AW854817 |
| 406740 | 0_0 | AA577274 AA908437 |
| 407644 | 27910_1 | D16815 AL563603 BG399756 AI935028 N41813 L31785 AL529953 AA428473 AA401262 BF802576 AA373868 BE886120 BM479352 |
| | | T28309 N57167 BI551531 BG037160 |
| 431788 | 71947_1 | AK054586 AL526339 BG257506 AW303347 AA594114 AA770237 AW117421 AA992677 AI028634 AA947172 AI242148 AA641234 |
| | | AL574323 AI219861 AI806256 AI033441 AA532785 AA905552 BE908245 N56134 H28597 R00255 AA969941 AA515532 AA887328 |
| | | AW104956 AI825261 AW207328 BE552249 AL562032 BI459483 BF447646 AW341155 |
| 419200 | 9531_1 | BF036043 AW190446 BG194731 AW662036 AI445021 BE937550 AW818972 AW393132 AA834685 BF112058 AV721682 H16423 |
| | | AI270167 AI857345 AA937302 AW818444 BE929780 BG498678 BF155010 BI598271 BI599811 BE161728 AW578737 AW753711 |
| | | AW379707 AW381918 BG506608 AW028637 AW994240 BF887392 BF790073 AW381624 AV727105 BF439618 AA443174 AI018009 |
| | | N42850 AW573242 AI417258 AA463483 AI676131 AI167170 AA836627 AA443828 AW592922 AA235129 AA730278 AW439062 AW474332 |
| | | BI043239 AW474342 BG708553 AW362423 BF090028 BE827256 R16550 R39478 R39479 R94368 BG540916 BM314745 AA251087 |
| | | D54231 D55274 BF085805 D31589 AW966405 AW994425 D81879 BE093545 AW901107 AA383529 BI021552 R56420 N39976 AA573281 H82595 AA234955 BE093539 |
| | | AW367006 BF358697 BF366318 AA663856 BE702099 BF035969 AI267384 AI267232 BE348320 AA621574 |
| | | AA861212 BF083343 BF083341 AV745131 D53074 AW954476 AW954472 AA376836 AV724531 D53063 C14928 AA093287 AA062638 |
| | | BG483558 BE940050 AA765954 T70171 BE938775 BE940057 D53502 AW373300 AL118798 BM128728 AA193411 AW444709 AW952455 |
| | | AI887612 BF431948 BI496876 AI264159 BM128481 AI624657 AI689301 AI969467 AA861685 AA251595 AA625761 AA872090 AI826790 |
| | | AA328366 BE827416 R75951 D56918 R68122 BE827384 AL118797 AI184164 AA164411 BI495332 BE858113 AI863860 H00660 T69849 |
| | | AW780389 C14667 BE934995 BI018652 R92801 AA164410 H00752 AW373305 AW373299 AW373302 |
| 420711 | 36528_1 | AK056357 BG704214 BG714527 BE409405 BE514455 BE408705 BM351826 BM049253 BM352137 AI659121 AW074357 AI674368 |
| | | AW207841 BE646201 BM453984 BE674342 H19667 AI458175 AA279585 AW438632 AI687141 AA433999 AI245284 AA366782 AI948491 |
| | | AW450917 AW450396 AI949813 BE217772 BE504863 AI628938 BF059575 AI564121 AI073854 AW510570 AI769861 AW235541 |
| | | AW771224 AW236106 AW074675 AI919535 AA977064 AI364994 AI631614 W58249 AI034276 BE169956 AA743584 H19668 AI222131 |
| | | AA883261 AI351534 F10472 AW466882 AA814964 W58250 BF036304 BF932025 BM474362 BG110744 AW964083 AW369992 F12868 BF842785 AA356665 BI091945 |
| | | BG059147 W04844 R18398 BE925184 BI050068 BI825078 BE784883 AW175845 AA216130 BI257636 |
| | | AA504328 AA713664 BF754840 AW235144 R38555 R42461 BG676504 T75135 R15365 BM012684 |
| 408283 | 645845_1 | BE141579 AW178109 BE141573 AW807502 AW807510 AW807228 AW807125 AW807374 AW807480 AW807486 AW845845 AW807500 |
| | | AW807477 AW807555 AW807208 AW807478 AW846124 AW807470 AW845864 BE141596 AW807465 |
| 455908 | 1526468_1 | BE156188 BE156298 BE156306 BE156374 BE156377 |
| 408338 | 647704_1 | AW867079 AW182772 AW867086 |
| 440028 | 598730_1 | AW473675 AI190744 W69997 AW104913 AI221098 W69996 AA885487 AA861491 |
| 427239 | 20459_2 | AL532360 BE794750 AA582906 AI015067 AW271034 BG271636 AW075177 AW071374 AI345565 AI307208 BE138953 BE049086 |
| | | AI334881 AW075006 AW075181 AA464019 AW302733 AW075100 AW073433 AI802854 AI334909 AI802853 AI345036 AI348921 AI340734 |
| | | AI307478 AI251289 AW302327 AW072520 AI312145 AW073656 AW072513 AW071289 AI307559 AA876186 T29587 AI307493 AI255068 |
| | | AI252868 AI252839 AW074809 AI252926 AI252160 AI251662 AI251262 AI610913 AI270787 AI270156 AI252075 AW073469 AW072901 |
| | | AW072496 AW071420 AI305762 AI254764 AI802837 AI251264 AW073049 AW071311 AI340643 BE138965 BE138502 AW073456 |
| | | AI334733 AI054335 BE139260 AI054302 AI054060 AI054057 AI053722 AI289711 BE139228 AW470478 AW271039 AW302085 BE041872 AI254494 AI271496 AI252427 |
| | | BF718773 BF718645 AW074866 BE857822 |
| 420762 | 9515_9 | BG121009 AA280094 Z44768 BE931923 AA249363 |
| 457133 | 775_1 | M54968 AA861543 AA356548 AI469981 NM_004985 BC013572 AW242010 AI740449 BF588730 AA425263 W21091 AI554920 T28976 |
| | | AW966530 BC010502 BM146110 AW504574 BG142117 AA232870 AI539465 AU123937 BI260541 BG261289 N24354 N32462 H08297 |
| | | F05452 AA281203 N80109 AA330460 D51551 BE178107 BE177839 BE178094 BE178141 BE931948 BE178093 BE178215 AL138354 |
| | | AI091259 AW020801 AA287991 BE178352 AA281183 BE178186 R70669 BI833742 BE178449 BE178043 W47526 BE178018 BE834453 |
| | | W93668 BE177936 AL576273 AU151852 AI004579 AW453052 AA287927 BM144801 AU155143 AA281094 AA737010 AA872481 |
| | | AA676681 AA843916 AI697660 AA564243 BF109372 AI015987 BF432094 AA937541 AI334416 AI281159 AA582189 AA553559 AW160515 |
| | | AA255527 AA670007 AA808271 AA281015 AA649252 BE464958 AW167917 BF436494 AA525301 AI889481 AI364302 AW173466 |
| | | AI676214 AA243624 AA648116 AA854442 AA721296 F01713 BE178360 BE178299 BE178460 BE178385 BE178424 AI301629 BE049265 |
| | | AU151277 AU155121 AU155337 AA889246 AU153181 AA342730 AW601642 H08199 R41588 R99351 H84241 W47527 R26447 R49696 |
| | | H02312 R79833 AV716970 AA262387 BE178441 BE178356 BE178100 BI491761 BE178467 BE931952 BF758698 BE177893 BE178336 |
| | | AA256711 W80934 BE178447 AV689618 BE178438 BE177844 AV659669 BG619554 AU134516 R35396 BE178029 R28455 AW368425 |
| | | BE178017 BE177999 R34165 R79640 BE178422 BF761849 BE178196 BE178443 |
| 418874 | 2326268_1 | T60872 T60906 |
| 441252 | 122_1 | NM_032315 BC004991 BM468629 AL583162 BE741793 BE303031 BE244077 BE302759 AL583161 AL572691 AI804708 AI129827 |
| | | AW872388 AI653103 AI151487 BE504355 AI671476 AW770976 AI206149 AI678224 BE468167 AI201164 AI200893 AA574128 AI991968 |
| | | AW194793 AA426113 AI220474 AI201028 AA757281 AA844723 AA398466 AA484977 BF059403 AI470557 AA435830 AW016602 |
| | | AI915990 BF356355 AL569855 AW978643 AW242882 AI823516 AW235374 BF059684 C20622 T66303 AI912746 AW339765 AA968541 |
| | | AL526958 BF433829 AA628313 AW578403 AI909734 N75265 AW274422 W02124 BG474952 |
| 424055 | 889171_1 | AA337234 AA334524 AA337774 AA337229 |
| 458335 | 2696571_1 | AI762479 AI479609 AI001070 |
| 445243 | 1786048_1 | AI762401 AI217439 C20714 |
| 410847 | 1061340_1 | AW807424 AW807090 AW807326 AW807172 AW807368 AW807079 AW807332 |
| 411051 | 7023_14 | BE145287 AW853931 AW814931 |
| 455211 | 1110641_1 | AW866450 AW866449 AW866306 |
| 400249 | 993_1 | BG260581 AI743827 AA182444 AA927609 NM_001326 U15782 BC010533 AA779834 AI086366 AI452475 AW274511 BG056719 |
| | | AW026350 AA808891 AW080007 AI763436 AU154714 AU155464 BF196839 AI934353 AI376072 BG232033 AI040445 AA700556 |
| | | AW004704 BE047781 AA470756 AI091381 AI302228 AI400050 AI142702 AA614554 AI467907 AA282801 AI434140 AI357496 AA748501 |
| | | AA430113 BF060907 AW207004 AI367341 AA873520 AA764823 AI077410 AA253061 AI052369 F08358 BM456285 BI518533 BI836074 |
| | | AU133365 AU131081 AU127466 AA173834 AW999116 BG571523 AU100072 BG290403 AI743461 W90141 D58551 AA181551 AA094014 |
| | | BG724183 |
| 439092 | 919640_1 | AW978407 AA830149 M85983 AW503637 BF352096 |
| 454838 | 1074031_1 | AW834044 AW833984 AW833985 AW834004 AW834065 AW833986 AW834076 AW834038 |
| 435542 | 132718_1 | AW975503 BE763276 H74234 AA687376 |
| 435784 | 135784_1 | AA778903 AA700146 AA705437 |
| 456381 | 250281_1 | AA236606 AA237079 AA459341 |
| 412636 | 1438_1 | M77830 NM_004415 AF139065 BG681115 BG740377 BI712964 BG000656 AA128470 BI438324 H27408 BE931630 BE167165 AW370827 |
| | | AW370813 J05211 BG698865 BG740734 BG680618 BG739778 BI765807 BM353403 BM353248 AW177784 AW205789 AW951576 |
| | | AW848592 BE182164 BF149266 BE940187 BI060445 BI060444 BF350983 BE720095 BE720069 BE715154 BE082584 BE082576 |
| | | BE004047 AA857316 BI039774 BE713818 BE713548 AW170253 BE160433 BI039775 AW886475 BM462504 BE931734 BF149264 |
| | | AA340777 BF381183 BG621737 AU127260 AW364859 BF993352 BG223489 BE819009 BF381184 BE715956 R58704 AA852212 |
| | | AW366566 BI090358 BF087707 BE819046 BE819005 AA377127 BE073467 BE819069 BE819048 BI036306 BG990973 BI040954 |
| | | BF919911 AU140155 AI951766 AI434518 AW804674 BF752969 BE837009 BE925826 BF149265 AW995615 BE814264 BI039782 AU140407 BE144243 BE709863 BF985642 |
| | | BE001923 BF933510 AW265328 BG436319 BE182166 AW365175 AW847688 BE818280 |
| | | AW177933 BF873679 AW178000 BE082526 BF476866 BF086994 BF592276 BE082507 BE082514 BE082505 BF873693 AW068840 |
| | | AW847678 BF804153 AW365157 BE813930 BE002030 AW365153 BE184941 BF749421 BE184920 BF839562 BE184933 BF842254 |
| | | BE698470 BE931048 BF999889 BF368816 BE184924 BE159646 BE714632 BE184948 BG986845 AA131128 AA099891 W39488 C04715 |
| | | BF096124 BE865341 AW799304 AL603116 BE149760 BE705967 BE705966 BE705968 AW848723 AW376699 AW376817 AW376697 |
| | | BG005097 BF751115 BE696084 AW848371 AW376782 AW848789 AW849074 AW361413 BF927725 BF094211 AW997139 BE865474 |
| | | BE185187 BE156621 BE715089 BE713297 BE713298 BE179915 AW799309 BF872345 BF088676 BE705939 AW752599 BG005197 |
| | | BF350086 BE715196 BE715155 BF752396 BF093817 BF831190 BF752409 BE006561 BG959922 BF094833 BF094748 BF094583 |
| | | AW377699 AW607238 BE082519 AW377700 BF349467 AI190590 AI554403 AI392926 AU158477 BI467252 AU159919 AI760816 BF082516 |
| | | AI439101 AA451923 AI340326 AI590975 BI791553 AI700963 AI142882 AA039975 AA946936 AA644381 BM314884 AA702424 AI417612 AW190555 AI220573 AI304772 |
| | | AI270345 AI627383 AA552300 AI911702 AW166807 AI346078 W95070 AA149191 AA026864 AI830049 |
| | | AW780435 AI078449 AI819984 AI858282 BI468588 AI860584 AI025932 AA026047 AA703232 AA658154 AA515500 AW192085 AA918281 |
| | | T77861 AI927207 AI205263 BF082491 AW021347 AI568096 BE939862 AA088866 D12062 AA056527 AA782109 W19287 W02156 |
| | | AW150038 AA022701 T87181 H44405 AI910434 BF082513 AI494069 AI270027 AI635878 AA128330 BG681425 BE706078 R20904 |
| | | BG680059 BG676647 BF764409 AA026654 AV745530 BI762796 BG287391 AW798780 BE706045 BE926470 AW799118 BF087996 |
| | | BE002273 AW879451 AI571075 BE067786 AV721320 AI022862 N29754 C03378 N84767 AA131077 H30146 BE714290 AI686869 |
| | | AI568892 AI915596 AW105614 AI887258 AI538577 BE926474 BE067737 BG319486 AA247685 AW798883 AW103521 BF989173 |
| | | AW860878 BE939707 BE185750 BE714064 BE713903 BE713868 BE713763 BG950164 BE713810 AW365151 BG955489 BE005272 |
| | | BF915937 AW365148 AI905927 BF992780 AW853812 BG954443 BI770853 BG679406 BG740832 BG681087 BG698430 AA455100 |
| | | T87267 BE696209 BE696210 BI089483 BE006273 BE872225 AW391912 BE925515 BG677012 BG741970 AA026480 BE705999 |
| | | BG677157 BE009090 BG681378 BE712291 BG961498 BG678984 BI040941 AA337270 AW384371 AW847442 BI058659 BE813665 |
| | | W95048 W25458 AW177786 AA025851 BE931733 BF154837 BG949393 BE714441 AW996245 BE711801 AI284090 BE064323 BE719390 BE940148 BG991212 BF375714 |
| | | BF349522 BG996267 T48793 BI013292 BE001925 AW365156 AW365154 AW606653 BF763109 |
| | | BE931637 BE167181 BE713879 BF354008 BF678726 H90899 AW365145 W38382 AI498487 |
| 454516 | 1058492_1 | AW803413 AW803403 AW803466 AW803268 AW803341 AW803402 AW803334 AW803355 AW803265 AW803414 AW803396 |
| 447782 | 2392961_1 | W29040 AI668903 AI424446 |
| 413645 | 1234345_1 | AA130992 AW969537 AA503835 |
| 410840 | 1061234_1 | AW806924 AW866478 AW866473 AW866390 AW866309 BF373982 AW866298 AW866539 AW866521 AW866547 AW866454 AW866537 |
| | | AW866517 BF373957 BF373949 AW866403 AW866369 |
| 437818 | 2512063_1 | AA769054 AA778197 |
| 406687 | 0_0 | M31126 |
| 400133 | 2368_1 | NM_005648 BC013809 L34587 BF103775 BG702618 BG716553 BI667090 BG505863 BF983483 BG718195 BI857891 BG501016 |
| | | BM043599 AL521812 BG705730 BI495545 BI495546 BF112248 BM023182 BM023123 AI075173 AW051799 BF058224 BI324885 |
| | | BF436008 AA398446 BG822375 BM019558 BM023382 BG164174 N56909 BI467064 BM023464 AI207475 BM311415 BG758430 |
| | | BG758807 AI934826 N90351 BG422026 BE910312 AI027778 AI081950 AI360890 BM009115 AI191829 BG759697 AI138728 AA399403 |
| | | AI355589 AI336427 AA868702 AA393660 AA025127 BG027630 AA962774 AA631224 BG940967 BE791087 AA573315 W81685 AA393525 |
| | | BG944103 AI339125 AI149864 AA977655 N90314 BE612839 BG491847 AI129091 AA461234 AA781198 AA759256 AA888954 AA975844 |
| | | AI184099 AI018025 AA398363 AI003331 AI193380 AA626020 AI244476 AI601114 AW135664 AI206607 AW263599 AA813219 AI684453 |
| | | AA878626 AA772222 AI085496 AI630226 BG940966 AI022010 AA770649 AA887624 AA491739 AA974295 BG530040 AA037091 |
| | | AA019912 BI160457 H64512 BG503896 |
| 400219 | 8366_1 | BC003552 L10284 NM_001746 AJ271880 BI834281 BI597016 AU133331 BI668332 BI463073 BG720694 AL046729 BI460138 BI461052 |
| | | AV647588 BG823268 AW889757 BE001258 AA312566 AA476446 AU098976 AA312196 BF749977 M94859 AF070646 BG564196 |
| | | BG623597 AU117332 BG680963 BI667083 AU134542 AU138830 AI761759 BG679882 AA209406 AW512644 AW514813 AI570535 |
| | | BE547592 BG655418 AI459204 BF725673 BE870032 BF001968 AL047245 BF724470 BF058818 H18415 BE076849 BE076857 BE076848 |
| | | BE076827 BE076856 AV708687 AA380923 BE076851 F08118 L18887 BF795701 AU128383 BE908383 AW673350 AW500108 BE079837 |
| | | BE929419 BF868758 AW503373 AW580528 BE005524 BI917505 BI457781 BE883812 BI668159 AA213643 AA374821 BF969974 BM480200 AU136152 BE395635 AV685066 |
| | | AV693755 AL040984 AA676820 BG896408 BE082272 BE082312 C05287 BE082264 |
| | | AA379850 AW672902 AI625955 BF033526 AW296557 AA728815 BE077058 AW081700 AA911707 AA362640 BE707179 BG913228 |
| | | BG116191 BG752367 AA074678 AL036937 BG113760 H03524 BG681802 AI564688 AU149556 BE178600 AI813488 AI452433 AI208989 |
| | | AA599392 AA580385 AA486274 AA629899 AA565929 AA114046 AA094252 BM450328 BG529968 R68320 BE076792 BE090073 |
| | | BE076855 BE076859 BE076829 AA361719 AA379164 AI202712 BG223315 BE122741 BG534531 AA903494 H24978 BF032674 BF085150 |
| | | BE739158 AA352904 C21593 BG697597 AA134969 AA374612 BE566182 BE871838 BE076911 AW579175 BF966390 BI548594 BG386452 BG913195 BM456787 N40286 |
| | | T80096 N39642 H42119 BG483861 AW381621 R69347 AW128895 AI367416 AI095285 AA099344 |
| | | BE568161 AA180109 BF246488 BI561938 AW579170 BG567212 |
| 412999 | 1343220_1 | BE046255 BE046611 BE046716 BE046732 BE046273 |
| 421258 | 266882_1 | AA286731 AA287621 AW188228 AW137774 BF902638 |
| 414390 | 2128_2 | BC022204 AK027690 NM_032839 BI765386 BE903404 BG700172 BF701671 BG252600 W60255 AW301576 W01296 AV724003 |
| | | BE999965 AI949788 BM040799 Z43693 BF082768 BF328302 H09192 BF332781 R34999 BG573394 N57281 BE009522 BE281040 |
| 458215 | 540112_3 | BE176902 BE177058 |
| 445511 | 9560_8 | BM471219 BE093160 BG171761 BI254009 AI905474 AA453162 AA829759 AI086559 AA776022 AI377446 BF589018 AA452822 |
| | | AW614566 AA443880 AA476733 AW970674 AI393291 AA988283 AI905528 AW384956 D78656 |
| 452470 | 40652_4 | AI903246 AI903467 AI903463 AI903248 AI903465 AI903243 |
| 439932 | 2601510_1 | AI589851 AI741578 AI359930 AI820062 AI742528 AA854682 AI038485 |
| 449016 | 1500732_1 | BE616111 BE081323 BE615864 |
| 423077 | 147641_1 | AW964587 BF737224 AA321699 AV740801 BI005365 N44978 |
| 459234 | 14095_−17 | AI940425 |
| 413402 | 151905_1 | T24065 BE092516 BE091995 BE092527 BE092284 BE092271 BE092204 BF332633 BE092280 BE092202 BE091991 BE091994 BF742886 |
| | | BF332627 BF332636 BE092068 BF742885 BE092063 BE092528 BE092447 BE092065 BE092283 BE092514 |
| 432492 | 715602_1 | AW275110 AA551054 AW867407 AW867499 BF374039 |
| 458422 | 4340_3 | NM_006260 BE048475 AW080036 AA287317 AA400028 AI204437 AI830642 AA644420 AW614662 AW261942 U28424 BG335330 |
| | | AU148480 AI095508 BF727387 BG256497 AI521859 AW291686 AW007816 BG002833 AA853075 AA779079 BF082050 AI640393 |
| | | AA522954 T55310 AW517649 AA127463 AA887984 AW206341 BE858004 AW772531 AA604169 H83777 BG290990 AI692188 AI223311 |
| | | AI708839 D82262 AA600260 AI364786 AA471007 |
| 434636 | 15423_1 | AJ420454 AF147430 AA910497 BF432963 AI701451 AI743089 AA429326 AI887812 AA315932 AI005464 AL043321 AI300993 AA425105 |
| | | BE467230 BE669770 AA885637 BE503044 AW014324 AI809584 AW167510 AA921331 AA903224 W01644 AI762128 AA031404 BE550653 |
| | | AI694045 BE043088 BE670430 AI630969 AI457315 BE644737 BE327316 AW295247 N92784 AI630807 BE328180 AI269949 AW245292 |
| | | AA083765 AA256898 AI375535 AA430673 AI168735 AI589717 AA015942 AI693885 AW341205 AA931651 BF856764 BE468094 BF433393 |
| | | BF445511 AA928976 AI817684 BF111008 AA428316 AA455858 N25716 AA568727 AI581817 AA427482 H40678 AA041483 N71630 |
| | | H51826 BF969052 AA094470 AI560352 T98937 W52816 AA083764 |
| 456487 | 8963_2 | AF064804 AA903211 AA829283 W05727 T30970 T32140 AI798807 BG218634 AW070452 AI767005 BF197394 BG202323 BG190077 |
| | | AA013172 T98542 AL567878 |

Pkey: Unique Eos probeset identifier number
CAT number: Gene cluster number
Accession: Genbank accession numbers

TABLE 12C

| Pkey | Ref | Strand | Nt_position |
|---|---|---|---|
| 401451 | 6634068 | Minus | 119926-121272 |
| 405770 | 2735037 | Plus | 61057-62075 |
| 401197 | 9719705 | Plus | 176341-176452 |
| 401519 | 6649315 | Plus | 157315-157950 |
| 406399 | 9256288 | Minus | 63448-63554 |
| 401866 | 8018106 | Plus | 73126-73623 |
| 404568 | 9966995 | Minus | 92893-93116 |
| 404561 | 9795980 | Minus | 69039-70100 |
| 403485 | 9966528 | Plus | 2888-3001, 3198-3532, |
| | | | 3655-4117 |
| 402812 | 6010110 | Plus | 25026-25091, 25844-25920 |
| 401464 | 6682291 | Minus | 170688-170834 |
| 406181 | 5923650 | Plus | 16586-16855 |
| 404996 | 6007890 | Plus | 37999-38145, 38652-38998, |
| | | | 39727-39872, 4055 |
| 402802 | 3287156 | Minus | 53242-53432 |
| 402408 | 9796239 | Minus | 110326-110491 |
| 402053 | 8083229 | Plus | 62703-63179 |
| 401558 | 7139678 | Plus | 103510-104090 |
| 402496 | 9797769 | Minus | 8615-9103 |
| 404704 | 9800728 | Minus | 88841-89018 |
| 403127 | 9211333 | Minus | 123813-124035 |
| 404632 | 9796668 | Plus | 45096-45229 |
| 404571 | 7249169 | Minus | 112450-112648 |
| 402114 | 8318586 | Plus | 71578-71715 |
| 405204 | 7230116 | Plus | 126569-126754 |
| 402855 | 9662953 | Minus | 59763-59909 |
| 403817 | 8962065 | Plus | 110297-111052 |
| 406081 | 9123861 | Minus | 38115-38691 |
| 406304 | 8575869 | Plus | 98839-98943, 105865-106005 |
| 400884 | 9958187 | Minus | 57979-58189 |
| 404995 | 6006247 | Minus | 154015-154123 |
| 400583 | 9887611 | Minus | 201148-201272 |
| 402537 | 9801061 | Minus | 111945-112220, 112466-112741 |
| 401560 | 8122921 | Plus | 94912-95082 |
| 404148 | 9863703 | Plus | 78218-78418, 79571-79709 |
| 404891 | 7329392 | Plus | 84974-85125 |
| 403416 | 9438737 | Plus | 21296-21453 |
| 405031 | 7533980 | Plus | 142468-142830 |
| 401216 | 8077122 | Minus | 154722-154944 |
| 403851 | 7708872 | Plus | 22733-23007 |
| 401203 | 9743387 | Minus | 172961-173056, 173868-173928 |
| 404440 | 7528051 | Plus | 80430-81581 |
| 405942 | 6758796 | Minus | 17579-18157 |
| 403011 | 6693597 | Minus | 3468-3623 |
| 403882 | 7710258 | Plus | 60805-60997, 62455-62559 |
| 403532 | 8076842 | Minus | 81750-81901 |
| 401435 | 8217934 | Minus | 54508-55233 |
| 400965 | 7770576 | Minus | 173043-173564 |
| 402025 | 7547159 | Plus | 173835-173998 |
| 404173 | 9930908 | Minus | 51303-51635 |
| 404819 | 4678240 | Plus | 16223-16319, 16427-16513, |
| | | | 16736-16859, 1694 |
| 402270 | 3108020 | Plus | 117656-117822 |
| 404607 | 7328770 | Plus | 28477-28591 |
| 405366 | 2182280 | Plus | 22478-22632 |
| 404624 | 9796168 | Minus | 92857-93081 |
| 404901 | 7331453 | Plus | 22679-22837, 27864-28108 |
| 406137 | 9166422 | Minus | 30487-31058 |
| 406303 | 8575868 | Plus | 173622-173786 |
| 403848 | 7708855 | Minus | 26695-26912 |
| 402261 | 2887496 | Plus | 2872-3032, 4673-4847, 12749-12886 |
| 400821 | 8570338 | Plus | 7188-7393 |
| 400857 | 9739340 | Minus | 44729-44873 |
| 403671 | 7272159 | Plus | 104461-104701 |
| 402280 | 2795792 | Plus | 16831-17570 |
| 405976 | 8247789 | Minus | 126103-126874 |
| 406203 | 7289992 | Minus | 82220-82639 |
| 402609 | 9926446 | Minus | 113464-113633, 114264-114654 |
| 402927 | 8217647 | Minus | 47247-47396 |
| 403324 | 8440025 | Minus | 107104-107309 |
| 400528 | 6981824 | Plus | 472381-472528, 474170-474277, |
| | | | 475328-47554 |
| 404478 | 8096498 | Minus | 65417-65625 |
| 402265 | 3287673 | Plus | 21059-21168 |
| 401796 | 7321653 | Minus | 36356-36533 |
| 401783 | 7249190 | Plus | 139369-139827, 140509-140591, |
| | | | 140834-14099 |
| 402553 | 9863566 | Plus | 48292-48398, 49564-49944 |
| 405558 | 1621110 | Plus | 4502-4644, 5983-6083 |
| 404576 | 4020145 | Plus | 17309-17938 |
| 401924 | 3892083 | Minus | 129614-129831, 130489-130614, |
| | | | 130696-13085 |
| 406389 | 9256209 | Minus | 48376-48504, 50635-50763, |
| | | | 52935-53063, 5601 |
| 404156 | 9886577 | Plus | 127319-127754 |
| 404580 | 6539738 | Minus | 240588-241589 |
| 405418 | 6997292 | Plus | 51839-51953 |
| 403291 | 7230870 | Plus | 95177-95435 |
| 400995 | 8099094 | Plus | 141186-141601 |
| 405357 | 2133873 | Minus | 4536-4604, 5297-5392 |
| 403055 | 8748904 | Minus | 109532-110225 |
| 402102 | 8117771 | Minus | 174566-174740 |
| 401759 | 9929699 | Plus | 59811-60665 |
| 405510 | 7630909 | Minus | 101028-101174 |
| 402842 | 9369121 | Minus | 76355-76479 |
| 401696 | 3417290 | Minus | 46209-46401 |
| 405484 | 5922025 | Plus | 199214-199579, 199672-199920, |
| | | | 200262-20049 |
| 405010 | 6094622 | Plus | 28830-29050 |
| 405704 | 4204244 | Plus | 138842-139051 |
| 403167 | 9838127 | Plus | 162599-162935 |
| 405276 | 4079595 | Minus | 40060-40215 |
| 403349 | 8569773 | Minus | 167815-168374 |
| 405451 | 7622517 | Minus | 145949-146227 |
| 403961 | 7596976 | Minus | 110393-110603 |
| 405863 | 7657810 | Plus | 49410-49620 |
| 405258 | 7329310 | Plus | 129930-130076 |
| 402412 | 7408036 | Plus | 75075-75679 |
| 400567 | 9884730 | Plus | 72252-72396 |
| 404109 | 9211742 | Minus | 144675-144928, 151899-152064 |
| 405723 | 9801668 | Plus | 114896-115831 |
| 404120 | 7342152 | Plus | 135775-136000 |
| 402285 | 2689079 | Minus | 92386-92634 |
| 400817 | 8569994 | Plus | 170793-170948 |
| 402505 | 9797871 | Plus | 27326-28231 |
| 405215 | 7230201 | Minus | 61683-61808 |
| 402008 | 6630597 | Plus | 164419-164809 |
| 405387 | 6587915 | Minus | 3769-3833, 5708-5895 |
| 405573 | 3820491 | Minus | 32645-33144 |
| 402663 | 8077020 | Minus | 14155-14364 |
| 401917 | 9502466 | Plus | 25054-25229 |
| 404494 | 8151634 | Minus | 53604-53893 |
| 400639 | 9887597 | Plus | 23150-23580 |
| 405705 | 4165009 | Plus | 120228-120800 |
| 404423 | 7407959 | Plus | 34438-34618 |
| 401913 | 9369520 | Minus | 33753-33904 |
| 405028 | 7533974 | Minus | 110588-110847, 110933-111115 |
| 402189 | 8576043 | Minus | 128318-129601 |
| 401804 | 7331475 | Minus | 154865-155024 |
| 402229 | 9965022 | Minus | 15739-15951, 16166-16779 |
| 404877 | 1519284 | Plus | 1095-2107 |
| 404341 | 7630856 | Minus | 13527-14130 |
| 401160 | 6067118 | Minus | 8280-8945 |
| 401925 | 3892083 | Minus | 138252-138469, 140239-140364, |
| | | | 140437-14059 |
| 406374 | 9256132 | Plus | 58991-59137 |
| 402530 | 7630937 | Minus | 1524-2003 |
| 403332 | 8568139 | Minus | 31409-31674 |
| 401952 | 3319121 | Minus | 53770-53979 |
| 402819 | 6729581 | Plus | 29217-29422 |
| 402014 | 7417799 | Plus | 36641-37171 |
| 403549 | 8081591 | Minus | 137150-137362 |
| 403481 | 9965004 | Plus | 93496-93633 |
| 401438 | 4885691 | Minus | 72461-72605 |
| 403429 | 9719566 | Minus | 52789-52917 |
| 403154 | 7407986 | Minus | 14228-14736 |
| 403858 | 7708944 | Plus | 37262-37517 |
| 400540 | 7574902 | Plus | 116601-116700, 119129-119312, |
| | | | 122026-12218 |
| 401403 | 7710966 | Plus | 146180-146294 |
| 406423 | 9256411 | Plus | 165600-165824 |
| 401711 | 6682593 | Minus | 19740-19938 |
| 402942 | 9368398 | Plus | 102152-102386 |
| 401138 | 2547238 | Plus | 3897-4121 |
| 406237 | 7417725 | Plus | 30032-30501 |
| 402322 | 7630359 | Minus | 75078-75203 |
| 404661 | 9797073 | Plus | 33374-33675, 33769-34008 |
| 405673 | 4589984 | Plus | 50700-50842 |
| 405869 | 6758731 | Minus | 89867-90358 |
| 406261 | 5686411 | Minus | 9906-10594 |
| 401708 | 2951946 | Plus | 154511-155298 |
| 402704 | 8782736 | Plus | 37368-37493 |
| 400987 | 8086488 | Minus | 22052-22185 |
| 404029 | 7671252 | Plus | 108716-111112 |
| 406596 | 8248613 | Minus | 128312-128539 |
| 405850 | 6164995 | Plus | 13871-14110 |
| 404745 | 6981829 | Plus | 101968-102920 |
| 401908 | 8698760 | Minus | 126888-127024 |
| 405365 | 2275192 | Minus | 119867-120372, 120481-120824, |
| | | | 121029-12135 |
| 405883 | 6758747 | Plus | 60621-61193 |
| 401095 | 9965511 | Plus | 140224-140501, 141208-141373 |
| 402840 | 9369121 | Minus | 57118-57306 |
| 406255 | 7417729 | Plus | 2959-3200 |
| 404306 | 5679453 | Plus | 32024-32174, 32823-32927 |
| 404053 | 3548785 | Plus | 61797-64205 |
| 400834 | 8705192 | Plus | 121963-122288 |
| 401192 | 9719502 | Minus | 69559-70101 |
| 405760 | 6066938 | Minus | 37424-38045 |
| 404071 | 7210053 | Minus | 167354-167859, 168810-168920, |
| | | | 169000-16910 |
| 404754 | 7637341 | Plus | 57788-57931, 59369-59525, |
| | | | 77040-77131, 7930 |
| 405710 | 5531256 | Minus | 66203-66832 |
| 405779 | 7280331 | Minus | 33048-33856 |
| 400880 | 9931121 | Plus | 29235-29336, 36363-36580 |
| 405697 | 4309923 | Minus | 56765-57010, 57696-58016 |
| 401898 | 8570008 | Minus | 72013-72132, 72400-72487 |
| 403509 | 7630896 | Plus | 144706-144901 |
| 401463 | 6682291 | Minus | 163135-163262 |
| 400730 | 8118960 | Plus | 11434-11748 |
| 403794 | 8096910 | Plus | 163292-163884 |
| 404496 | 8151634 | Plus | 145065-145179 |
| 400547 | 9801025 | Plus | 41505-42032 |
| 406542 | 7711499 | Plus | 117335-118473 |
| 401405 | 7768126 | Minus | 69276-69452, 69548-69958 |
| 400845 | 9188605 | Plus | 34428-34612 |
| 403935 | 7454203 | Plus | 16955-17310 |
| 404705 | 9800794 | Minus | 75671-75910 |
| 404592 | 9943965 | Minus | 39067-39225 |
| 402239 | 7690131 | Plus | 38175-38304, 42133-42266 |
| 402889 | 9931133 | Plus | 89392-89498, 90358-90571 |
| 400750 | 8119067 | Plus | 198991-199168, 199316-199548 |

Pkey: Unique number corresponding to an Eos probeset
Ref: Sequence source. The 7 digit numbers in this column are Genbank Identifier (GI) numbers. “Dunham I. et al.” refers to the publication entitiled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

1. A method of detecting a metastatic breast cancer or metastatic lung cancer-associated transcript in a cell, the method comprising contacting a biological sample with a polynucleotide that selectively hybridizes to a nucleic acid sequence at least 80% identical to a sequence selected from those listed in Tables 1-12.

2. The method of claim 1, wherein the metastatic cancer-associated transcript is a metastatic lung cancer-associated transcript.

3. The method of claim 1, wherein the metastatic cancer-associated transcript is a metastatic breast cancer-associated transcript.

4. The method of claim 1, wherein the biological sample comprises isolated nucleic acids.

5. The method of claim 1, wherein the polynucleotide or the biological sample is labeled.

6. The method of claim 1, wherein the polynucleotide is immobilized on a solid surface.

7. An isolated nucleic acid molecule consisting of a polynucleotide sequence selected from those listed in Tables 1-12.

8. An expression vector comprising the nucleic acid of claim 7.

9. A host cell comprising the expression vector of claim 8.

10. An isolated polypeptide which is encoded by a nucleic acid sequence selected from those listed in Tables 1-12.

11. An antibody that specifically binds a polypeptide of claim 10.

12. The antibody of claim 11, wherein the antibody is an antibody fragment.

13. The antibody of claim 11, wherein the antibody is a humanized antibody

14. A method of detecting a metastatic breast cancer or metastatic lung cancer cell in a biological sample, the method comprising contacting the biological sample with an antibody of claim 11.

15. The method of claim 14, wherein the antibody is labeled.

16. A method of detecting antibodies specific to metastatic breast cancer in a patient, the method comprising contacting a biological sample from the patient with a polypeptide encoded by a nucleic acid comprising a sequence selected from those listed in Tables 1A-5C, 11A-12C.

17. A method of detecting antibodies specific to metastatic lung cancer in a patient, the method comprising contacting a biological sample from the patient with a polypeptide encoded by a nucleic acid comprising a sequence selected from those listed in Tables 6A-12C.

18. A method for identifying a compound that modulates a metastatic breast cancer-associated polypeptide, the method comprising the steps of:
(i) contacting the compound with a metastatic breast cancer-associated polypeptide, the polypeptide encoded by a polynucleotide that selectively hybridizes to a nucleic acid sequence at least 80% identical to a sequence selected from those listed in Tables 1A-5C, 11A-12C; and
(ii) determining the functional effect of the compound upon the polypeptide.

19. The method of claim 18, wherein the functional effect is determined by measuring ligand binding to the polypeptide.

20. A method for identifying a compound that modulates a metastatic lung cancer-associated polypeptide, the method comprising the steps of:
(i) contacting the compound with a metastatic breast cancer-associated polypeptide, the polypeptide encoded by a polynucleotide that selectively hybridizes to a nucleic acid sequence at least 80% identical to a sequence selected from those listed in Tables 6A-12C; and
(ii) determining the functional effect of the compound upon the polypeptide.

21. A method of inhibiting proliferation of a metastatic breast cancer-associated cell in a patient, the method comprising the step of administering to the subject a therapeutically effective amount of a compound that modulates a polypeptide encoded by a nucleic acid sequence selected from those listed in Tables 1A-5C, 11A-12C.

22. A method of inhibiting proliferation of a metastatic lung cancer-associated cell in a patient, the method comprising the step of administering to the subject a therapeutically effective amount of a compound that modulates a polypeptide encoded by a nucleic acid sequence selected from those listed in Tables 6A-12C.

23. A drug screening assay comprising the steps of
(i) administering a test compound to a mammal having metastatic breast cancer or a cell isolated therefrom;
(ii) comparing the level of gene expression of a polynucleotide that selectively hybridizes to a sequence at least 80% identical to a sequence selected from those listed in Tables 1A-5C, 11A-12C in a treated cell or mammal, with the level of gene expression of the polynucleotide in a control cell or mammal, wherein a test compound that modulates the level of expression of the polynucleotide is a candidate for the treatment of metastatic breast cancer.

24. A pharmaceutical composition for treating a mammal having metastatic breast cancer, the composition comprising a compound identified by the assay of claim 23 and a physiologically acceptable excipient.

25. A drug screening assay comprising the steps of
(i) administering a test compound to a mammal having metastatic lung cancer or a cell isolated therefrom;
(ii) comparing the level of gene expression of a polynucleotide that selectively hybridizes to a sequence at least 80% identical to a sequence selected from those listed in Tables 6A-12C in a treated cell or mammal, with the level of gene expression of the polynucleotide in a control cell or mammal, wherein a test compound that modulates the level of expression of the polynucleotide is a candidate for the treatment of metastatic lung cancer.

26. A pharmaceutical composition for treating a mammal having metastatic lung cancer, the composition comprising a compound identified by the assay of claim 25 and a physiologically acceptable excipient.

27. A method of detecting a metastatic breast cancer-associated polypeptide in a cell, the method comprising contacting a biological sample from the patient with a antibody that that specifically binds a polypeptide encoded by a polynucleotide sequence selected from those listed in Tables 1A-5C, 11A-12C.

28. The method of claim 27, wherein the antibody is labeled.

29. A method of detecting a metastatic lung cancer-associated polypeptide in a cell from a patient, the method comprising contacting a biological sample from the patient with a antibody that that specifically binds a polypeptide encoded by a polynucleotide sequence selected from those listed in Tables 6A-12C.

30. The method of claim 29, wherein the antibody is labeled.
